(19) United States
(12) Patent Application Publication
(10) Pub. No.: US 2026/0088124 A1
(43) Pub. Date: Mar. 26, 2026

(54) TRANSCRIPTOMIC CLOCKS OF BIOLOGICAL AGE AND LIFESPAN

(71) Applicant: The Brigham and Women's Hospital, Inc., Boston, MA (US)

(72) Inventors: Vadim N. Gladyshev, Brookline, MA (US); Aleksandr Tyshkovskii, Cambridge, MA (US)

(21) Appl. No.: 19/106,681

(22) PCT Filed: Sep. 1, 2023
(86) PCT No.: PCT/US2023/031899

§ 371 Date: Feb. 26, 2025

Related U.S. Application Data
(60) Provisional application No. 63/374,290, filed on Sep. 1, 2022.

(51) Int. Cl. G16B 25/10; C12Q 1/68; G16B 20/00; G16B 40/20; G16H 10/40; G16H 10/60; G16H 50/30

(52) U.S. Cl. CPC G16B 25/10; C12Q 1/68; G16B 20/00; G16B 40/20; G16H 10/40; G16H 10/60; G16H 50/30; C12Q 2600/118

(57) ABSTRACT

Provided herein are methods that use gene transcription levels to quantify mammalian expected total lifespan, remaining lifespan, chronological age and lifespan-adjusted biological age and predict whether an intervention will be protective or damaging to lifespan and health outcomes.

### CLAIM OF PRIORITY

This application claims the benefit of U.S. Provisional Application Ser. No. 63/374,290, filed on Sep. 1, 2022. The entire contents of the foregoing are incorporated herein by reference.

### FEDERALLY SPONSORED RESEARCH OR DEVELOPMENT

This invention was made with Government support under Grant Nos. AG067782, AG047200, and AG065403 awarded by the National Institutes of Health. The Government has certain rights in the invention.

### TECHNICAL FIELD

Provided herein are methods that use gene transcription levels to quantify mammalian expected total lifespan, remaining lifespan, chronological age and lifespan-adjusted biological age and predict whether an intervention will be protective or damaging to lifespan and health outcomes.

### BACKGROUND

Understanding mechanisms of aging and longevity is crucial for the development of geroprotectors.

### SUMMARY

Provided herein are computer-implemented methods for calculating expected total and remaining lifespan, chronological age, and lifespan-adjusted age of a biological test system, optionally a cell, tissue, organ, or organism. The methods include providing a biological test system, optionally a cell, tissue, organ, or organism; determining expression of two or more transcripts identified in Table A; accessing, from memory, a model for calculating lifespan, chronological age, or lifespan-adjusted age of a biological test system, wherein the model calculates lifespan, chronological age, or lifespan-adjusted age, i.e. chronological age of the organism normalized by its expected lifespan, based on expression of the two or more transcripts; and calculating, using the model, expected lifespan, chronological age, or lifespan-adjusted age for the system, e.g., expressed as a value ranging from 0 (birth) to 1 (end of life).

In some embodiments, the methods comprise determining expression of 5, 10, 20, 30, 40, 50, 60, 70, 80, 90, 100, 125, 150, or more of the transcripts. In some embodiments, the methods comprise expression of up to 100, 125, 150, 175, 200, 250, 300, 350, 400, or 500 of the transcripts.

In some embodiments, the methods further comprise applying an intervention to the system, determining expression of the one or more transcripts during and/or after an application of an intervention, and calculating chronological age, lifespan-adjusted age, or expected lifespan based on the expression of the one or more transcripts.

In some embodiments, the methods further comprise comparing the chronological age, lifespan-adjusted age, or expected lifespan to a reference chronological age, lifespan-adjusted age, or expected lifespan. In some embodiments, the reference chronological age, lifespan-adjusted age, or expected lifespan is a baseline chronological age, lifespan-adjusted age, or expected lifespan obtained in the same test system before application of an intervention, or a chronological age, lifespan-adjusted age, or expected lifespan obtained earlier in time in the same test system, or a chronological age, lifespan-adjusted age, or expected lifespan in a reference system that represents the chronological age, lifespan-adjusted age, or expected lifespan in the absence of an intervention.

In some embodiments, chronological age, lifespan-adjusted age, or expected lifespan is calculated using a software-based modeling algorithm, optionally wherein the algorithm comprises a linear algorithm; principal component analysis (PCA); classification or decision trees; elastic net analysis; linear and polynomial support vector machines (SMV); shrunken centroids; random forest algorithms; support vector machines; gradient boosting; or neural networks.

In some embodiments, the methods comprise calculating a chronological age, lifespan-adjusted age, or expected lifespan using the determined expression and applying an algorithm to the levels. In some embodiments, the algorithm comprises:

$$tAge = intercept + b 1 * T 1 + b 2 * T 2 + … + bn * Tn ;$$

Where b1-bn are the pre-trained model coefficients for genes listed in Table A. and T1-Tn are the normalized expression levels of given Transcripts. In some embodiments, the model is a log-log model, and the method further comprises performing a transformation wherein:

$${tAge}_{m} = 10 ** ( - 10 ** ( - tAge ) ) ,$$

wherein tAge is the estimate from the formula above, and tAgeₘ is a transformed tAge that reflects the chronological or lifespan-adjusted biological age on a scale of 0-1, where 0 is a moment of birth, and 1 is a maximum achievable lifespan for the model.

In some embodiments, the methods further comprise identifying an intervention as having a positive effect when expected lifespan increases, and/or chronological age, or lifespan-adjusted age decreases, and/or identifying an intervention as having a damaging effect when expected lifespan decreases, and/or chronological age, or lifespan-adjusted age increases.

In some embodiments, the methods further comprise selecting an intervention that has been identified as having a positive effect as a candidate intervention; applying the candidate intervention to an in vivo model of, optionally wherein the model is a non-human test animal or a human subject with a disorder or condition associated with aging; and determining whether the candidate intervention has a positive effect on the model, optionally on the disorder or condition related to aging.

Also provided herein are methods for predicting an effect of an intervention on lifespan. The methods comprise: providing a biological test system, optionally a cell, tissue, organ, or organism; determining expression of two or more transcripts identified in Table A; applying an intervention to the system, calculating chronological age, lifespan-adjusted age, or expected lifespan using a method described herein; comparing the predicted chronological age, lifespan-adjusted age, or expected lifespan to a reference chronological age, lifespan-adjusted age, or expected lifespan; and identifying an intervention as likely to have a protective effect on lifespan when chronological age or lifespan-adjusted age decreases and/or expected lifespan increases, and/or identifying an intervention as likely to have a damaging effect on lifespan when chronological age or lifespan-adjusted age increases and/or expected lifespan decreases.

In some embodiments, the methods comprise determining expression of 5, 10, 20, 30, 40, 50, 60, 70, 80, 90, 100, 125, 150, or more of the transcripts. In some embodiments, the methods comprise determining expression of up to 100, 125, 150, 175, 200, 250, 300, 350, 400, 500, or 1,000 transcripts, including determining expression of at least 5, 10, 20, 30, 40, 50, 60, 70, 80, 90, 100, 125, 150, or all of the transcripts. In some embodiments, the reference chronological age, lifespan-adjusted age or expected lifespan is a baseline chronological age, lifespan-adjusted age or expected lifespan obtained in the same test system before application of an intervention, or a chronological age, lifespan-adjusted age or expected lifespan obtained earlier in time in the same test system, or a chronological age, lifespan-adjusted age or expected lifespan in a reference system that represents the chronological age, lifespan-adjusted age or expected lifespan in the absence of an intervention.

In some embodiments, the methods comprise calculating chronological age, lifespan-adjusted age or expected lifespan using an algorithm, optionally wherein the algorithm comprises using a manual or software-based modeling algorithm, optionally wherein the algorithm comprises a linear algorithm; principal component analysis (PCA); classification or decision trees; elastic net analysis; linear and polynomial support vector machines (SMV); shrunken centroids; random forest algorithms; support vector machines; gradient boosting; or neural networks.

In some embodiments, the methods comprise calculating a chronological age, lifespan-adjusted age or expected lifespan using the determined expression and applying an algorithm to the levels. In some embodiments, the algorithm comprises:

$$tAge = intercept + b 1 * T 1 + b 2 * T 2 + … + bn * Tn$$

Where b1-bn are the pre-trained model coefficients for every gene from Table A. and T1-Tn are the normalized expression levels of given Transcripts.

In some embodiments, the model is a log-log model, and the method further comprises performing a transformation wherein:

$${tAge}_{m} = 10 ** ( - 10 ** ( - tAge ) ) ,$$

wherein tAge is the estimate from the formula above, and tAgeₘ is a transformed tAge that reflects the chronological or lifespan-adjusted biological age on a scale of 0-1, where 0 is a moment of birth, and 1 is a maximum achievable lifespan for the model.

In some embodiments, the methods further comprise selecting an intervention that has been identified as having a protective effect as a candidate intervention; applying the candidate intervention to an in vivo model of a disorder or condition associated with aging, optionally wherein the model is a non-human test animal or a human subject in a clinical trial, e.g., of a disorder or condition associated with aging; and determining whether the candidate intervention has a protective effect on the model, e.g., on the disorder or condition related to aging.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-H. Transcriptomic clocks predict chronological and biological age of rodents.

(A) Scheme of data used for the aging clocks construction. 2,628 mouse and rat samples from 26 tissues and 85 independent datasets were used to construct tissue-specific and multi-tissue chronological aging clocks. 3,338 samples corresponding to control mice and mice subjected to different genetic, pharmacological, and environmental interventions were used to build tissue-specific and multi-tissue lifespan-adjusted aging clocks.

(B) Distribution of chronological age and tissue across samples used for construction of multi-tissue clock.

(C) Distribution of lifespan-adjusted age and model of intervention across samples used for construction of multi-tissue biological aging clock. Chronological age divided by the maximum lifespan of a given rodent model is shown on x axis. Strains of control mice and types of interventions applied to treated mice are reflected in color.

(D) Quality of multi-tissue clock of absolute chronological age. Every dot denotes a sample from training or test set. Pearson's correlation coefficient and mean absolute error (MAE) for test set is shown in text.

(E) Quality of multi-tissue clock of differential chronological age. For every dataset, random control group of the same age was chosen as a reference for prediction of chronological age difference. Every dot denotes a sample from training or test set. Pearson's correlation coefficient and mean absolute error (MAE) for test set is shown in text.

(F) Quality of multi-tissue chronological clock in independent datasets. Test set was compiled from datasets not included in the training set. Every dot denotes a sample from training or test set. Pearson's correlation coefficient and median absolute error (MAE) for test set is shown in text.

(G) Distribution of aging clock quality across 25 random subsamplings. Distribution of Pearson's correlation coefficient between real and predicted chronological age for test set is shown for brain, liver, skeletal muscle, and multi-tissue clocks. For every tissue, quality of absolute age clock (left), differential age clock (middle) and differential age clock on independent datasets (right) were tested.

(H) Transcriptomic age of mice subjected to Hutchinson-Gilford progeria syndrome (HGPS) according to chronological and biological gene expression clocks. Wildtype and mutant mice are shown in color. tAge for every biological sample was calculated with chronological liver clock (left) and lifespan-adjusted liver clock (right). Every dot represents individual mouse. Unpaired t-test p-value is shown above boxplots. WBC: White blood cells; SCAT: Subcutaneous adipose tissue; BAT: Brown adipose tissue; GAT: Gonadal adipose tissue; MAT: Mesenteric adipose tissue; WAT: White adipose tissue; Muscle: Skeletal muscle; MAE: Mean absolute error; A age: difference in age.

FIGS. 2A-G. Biological age of mice subjected to heterochronic parabiosis and sleep deprivation.

A) tAge of young and old mice subjected to isochronic and heterochronic parabiosis according to liver chronological clock. tAge of attached mice and mice after 3 months of detachment have been measured. The age of paired mouse is shown in color. Unpaired t-test p-values are shown in text.

B) Pearson correlation between Δ age estimated using epigenetic chronological multi-tissue and liver clocks (upper) and transcriptomic chronological and lifespan-adjusted liver clocks (bottom). Horvath universal clocks were used to calculate epigenetic age. Δ age were calculated by subtracting real age from eAge or tAge prediction.

C) Dependence between tAge and eAge estimated using chronological liver clocks. Different groups of parabiosis mice are shown in color. Attachment status is denoted by shape. Red line reflects linear dependence between tAge and eAge. Dotted line reflects identity line. Pearson's correlation coefficient and p-value of slope coefficient are shown in text.

D) Dependence between Δ age estimated using epigenetic and transcriptomic chronological liver clocks. Different groups of parabiosis mice are shown in color. Attachment status is denoted by shape. Red line reflects linear dependence between tAge and eAge. Pearson's correlation coefficient and p-value of slope coefficient are shown in text.

E) Scheme of sleep deprivation (SD) experiment.

F) Transcriptomic ages (tAges) of control mice (WT), mice subjected to 5 and 10 days of sleep deprivation (SD), and mice subjected to 10 days of SD and 5 days of recovery. ** p.adjusted<0.01, * p.adjusted <0.05.

G) Functional enrichment analysis (GSEA) of aging-associated genes affected by sleep deprivation compared to control (WT→SD), recovery compared to SD (SD→Rec), and SD compared to both control and recovery (WT+Rec→SD). Only functions significantly enriched by at least one signature are shown (adjusted p-value <0.1).

FIGS. 3A-G. Transcriptomic clocks capture aging-associated changes induced by age-related diseases, cellular reprogramming and during embryogenesis.

A) Difference in transcriptomic age between healthy mice and mice with induced ischemic stroke. tAge was calculated using lifespan-adjusted brain (for brain data, left) and multi-tissue (for heart data, right) clocks. Unpaired t-test p-value and GEO ID are shown in text.

B) Difference in brain transcriptomic age between healthy rats and rats with induced ischemic stroke. tAge was calculated using lifespan-adjusted brain clock. Samples from the same (ipsilateral) and the opposite (contralateral) hemispheres have been used for tAge prediction. Unpaired t-test p-value and GEO ID are shown in text.

C) Difference in transcriptomic age between healthy mice and mice with the model of 5xFAD Alzheimer's disease. tAge was calculated using lifespan-adjusted brain (for brain samples, left) and multi-tissue (for pineal gland samples, right) clock. Unpaired t-test p-value and GEO ID are shown in text.

D) Difference in kidney transcriptomic age between healthy mice and mice with chronic kidney disease. tAge was calculated using lifespan-adjusted multi-tissue clock. Unpaired t-test p-value and GEO ID are shown in text.

E) Difference in liver transcriptomic age between healthy mice and mice with hepatocarcinoma. tAge was calculated using lifespan-adjusted liver clock. Unpaired t-test p-value and GEO ID are shown in text.

F) Dynamics of tAge during murine embryogenesis. tAge was calculated using lifespan-adjusted multi-tissue clock. Unsmoothed mean tAge calculated for every time point and mean tAge smoothed using moving average approach are shown in grey and purple, respectively. Ground zero stage corresponding to the minimum epigenetic age according to previous studies is shaded in grey. Fertilization and birth time points are visualized with dotted lines. Data are means and error bars are±SE.

G) tAge of prefrontal cortex, primary fibroblasts, and OCT4, SOX2, KLF4, and MYC (collectively OSKM)-induced pluripotent stem cells (iPSCs) derived from humans with different chronological age. For every cell type, linear dependence between tAge and chronological age was assessed with Pearson's correlation coefficient and p-value, shown with the corresponding color.

IS: Ischemic stroke; AD: Alzheimer's disease; CKD: Chronic kidney disease; HCC: Hepatocarcinoma.

FIGS. 4A-E. Transcriptomic clocks capture aging of single cells

A) Quality of chronological age prediction on murine meta-cells from different organs. For each organ, meta-cell data was aggregated from randomly chosen 500 cells for every sample. tAge was calculated using multi-tissue chronological clock. Every dot reflects a single biological sample. Tissues are denoted by color. Pearson's correlation coefficient and adjusted p-value of slope coefficient (p.adj) are shown in text.

B) Dependence of quality of tAge prediction on number of aggregated cells. Meta-cell data for every tissue was aggregated using different number of randomly chosen cells (x axis). For every tissue, tAge was calculated using multi-tissue chronological clock. Pearson's correlation between predicted and real ages is shown on y axis.

C) Dependence of quality of tAge prediction on average read coverage within meta-cell. For every cell number shown on (B), average read coverage was calculated.

D) Chronological (left) and lifespan-adjusted (right) tAge across cell types. For every cell type, meta-cells were aggregated from randomly chosen non-overlapping 50 cells corresponding to young 1-month-old mice. Boxplots show distribution of meta-cell tAges for every individual cell type. ANOVA p-value is shown in text.

E) Age-related slope of chronological (left) and lifespan-adjusted (right) tAge for individual cell types. Meta-cells corresponding to different cell types and age groups were aggregated from randomly chosen non-overlapping 50 cells. Linear regression slope of tAge was calculated for each cell type. Adjusted p-values are denoted with asterisks. Bars are mean slope±SE and are colored based on adjusted p-value. * Adjusted p-value <0.05; ** adjusted p-value <0.01; *** adjusted p-value <0.001.

FIGS. 5A-E. Transcriptomic clocks reveal diverse trajectories of aging dynamics during reprogramming

A) FLE visualization of scRNA-seq profiles of cells subjected to OSKM reprogramming colored based on lifespan-adjusted tAge. tAge was calculated using lifespan-adjusted multi-tissue clock for every cell using lifespan-adjusted multi-tissue clock and visualized using color map.

B) FLE visualization of scRNA-seq profiles of cells subjected to OSKM reprogramming colored based on gene expression signatures of pluripotency, cell cycle, apoptosis and SASP. The average normalized expression of signature-associated genes is shown in color.

C) Lifespan-adjusted tAge dynamics along trajectories to different cell types during reprogramming. Trajectories towards different cell types were calculated using optimal transport method. For every trajectory, mean tAge across time was smoothed using moving average approach.

D) Gene signature dynamics along trajectories to different cell types during reprogramming. Average scores of gene signatures of pluripotency, cell cycle, apoptosis and SASP were calculated for each trajectory using optimal transport method. For every trajectory, mean scores across time were smoothed using moving average approach.

E) Correlation between lifespan-adjusted tAge and gene expression signatures of various features of cells subjected to OSKM reprogramming. Pearson's correlation coefficient was calculated for lifespan-adjusted tAge and gene expression signatures across all cells in the dataset. Gene signatures with absolute correlation coefficient >0.1 are shown in color. Data are means and error bars are±SE.

IPS: induced pluripotent cells; MET: mesenchymal-epithelial transition; FLE: Force-directed layout; SASP: Senescence-associated secretory phenotype.

FIGS. 6A-H. Transcriptomic clocks of aging and lifespan predict new longevity interventions

(A) Change of biological age induced by genetic, dietary and pharmacological interventions in mouse tissues. Change of tAge was calculated using lifespan-adjusted multi-tissue clock. Adjusted p-value (in log scale) calculated using unpaired t-test is plotted on y axis. Interventions with statistically significant effect on tAge are shown in color.

(B) Biological age of liver from young and old control mice, and old mice subjected to ouabain. tAge was calculated using lifespan-adjusted multi-tissue clock. Benjamini-Hochberg adjusted p-values calculated using unpaired t-test are shown in text.

(C) Distribution of maximum lifespan liver clock quality across 25 random subsamplings. Distribution of Pearson's correlation coefficient between real and predicted maximum lifespan for a given mouse model (defined as average lifespan of 10% most long-lived mice in the group) are shown on the x axis. Pearson's correlation coefficient and mean absolute error (MAE) for test set is shown in text.

(D) Predicted maximum lifespan of control mice and Little mice (left) or Ames dwarf mice (right) of different age. Predicted maximum lifespan was calculated using liver clock. Long-lived mice are shown (Ghrhr^{lit/lit} and Prop1^{df/df}). Statistical significance of difference in predicted lifespan between control and long-lived mice assessed with unpaired t-test p-value is shown with asterisks.

(E) Predictions of change in maximum lifespan induced by compounds tested in mice for 1 month for liver, kidney and pooled across tissues (right). Predictions are normalized based on the average value of control samples and sorted by the average effect across tissues. Statistically significant effects (t-test adjusted p-value <0.1) are shown in red. Average predictions for control samples are shown in black. Compounds chosen for a survival study that extended and not extended lifespan of old male C57Bl/6J mice are labeled in grey and black, respectively. Data are means and error bars are±SE.

(F-H) Survival curves of old male C57Bl/6J mice subjected to KU-0063794 (F), selumetinib (G) and AZD-8055 (H). Survival curves of control (to the left) and treated mice (to the right) are shown. Log-rank test p-value is shown in text.

FIGS. 7A-C. Age-associated molecular changes induced by age-related diseases, sleep deprivation and during early embryogenesis.

(A) Functional enrichment of age-associated gene expression changes induced by age-related diseases. Overlap of genes differentially expressed (adjusted p-value <0.05) in response to a particular age-related disease and genes with disease-induced pro-aging expression change according to the transcriptomic clock was utilized. Gene sets from Hallmarks ontology were used, and Fisher exact test was utilized to assess statistical significance of enrichment. Size of the dot reflects ratio of genes corresponding to the particular function, while its intensity denotes statistical significance (log of adjusted p-value).

(B) Epigenetic ages (eAges) of control mice (WT), mice subjected to 5 and 10 days of sleep deprivation (SD), and mice subjected to 10 days of SD and 5 days of recovery. eAges were calculated based on Horvath's pan-mammalian Universal Clock 2.

(C) Functional enrichment of anti-aging gene expression changes occurring before ground zero (day 8) (left) and pro-aging gene expression changes occurring after ground zero (right). Overlaps of genes with significant expression change (linear model adjusted p-value <0.05) before or after ground zero and genes with anti- or pro-aging expression changes according to the clock, respectively, were utilized. Gene sets from Hallmarks ontology were used, and Fisher exact test was utilized to assess statistical significance of enrichment. Size of the triangle reflects statistical significance (log of adjusted p-value), while its direction corresponds to the up- or downregulation of genes enriched by the corresponding function.

FIG. 8 is a schematic diagram of an example computer system.

### DETAILED DESCRIPTION

Described herein are robust transcriptomic clocks of biological age and longevity, built by performing and aggregating gene expression data for 3,338 mice and rats of different ages and expected lifespan across 26 tissues and 85 datasets. The clocks have diverse applications, including capturing changes in biological age due to chronic diseases, sleep deprivation, lifespan-extending interventions, cell reprogramming, and heterochronic parabiosis. When applied to scRNA-seq data, the clocks revealed cell type-specific age-related changes and different trajectories of age dynamics in cells subjected to OSKM. The lifespan clock also uncovered aging-independent mechanisms of longevity and predicted novel longevity interventions that indeed extended lifespan in a model mammal.

### Test Systems

Thus, provided herein are methods that can be used to monitor effects of various interventions on gene expression that affect longevity and health, and to identify interventions that can reduce or extend lifespan or healthspan in a subject.

The methods can be practiced using a biological test system, including one or more human cells, all or part of a human tissue, or all or part of a human organ. The cell can be, e.g., a mammalian cell, such as a primary cell (including erythrocytes; platelets; peripheral blood mononuclear cells (PBMC), e.g., lymphocytes, monocytes, or macrophages; bone marrow cells; endothelial cells, e.g., vascular or bronchial endothelial cells; pancreatic islet beta cells; renal cells; hepatocytes; neurons and glia; epidermal cells; respiratory interstitial cells; adipocytes; dermal fibroblasts; muscle cells; cells of the eye (e.g., photoreceptors, RPE cells, retinal ganglia cells) or ear (e.g., hair cells or supporting cells); or hair follicles. Primary or cultured cells including stem cells and immortalized cells can also be used, e.g., induced pluripotent stem cells (iPSCs), embryonic stem cells (ES cells), hematopoietic stem cells (HSCs), mesenchymal stem cells (MSCs), pre-adipocytes, and neural progenitor cells. Cultured cells such as HEK293 and fibroblasts can also be used. In some embodiments, the test system is a mouse or rat, or comprises liver or muscle cells from a rat, mouse, or human.

The tissues can be, e.g., connective tissue, epithelial tissue, muscle tissue, and nervous tissue. The organs can be, e.g., capillaries; joints; nerves; skin; tendons; arteries; cerebellum; liver; nasal cavity; spleen; tongue; appendix; diaphragm; lungs; ovaries; scrotum; thyroid; adrenal glands; ears; larynx; esophagus; stomach; trachea; brain; eyes; ligaments; penis; spinal cord; thymus gland; bones; fallopian tubes; lymph nodes; pancreas; small intestine; ureters; bronchi; genitals; large intestine; pharynx; salivary glands; urethra; bladder; gallbladder; lymphatic vessel; placenta; skeletal muscles; uterus; bone marrow; heart; mouth; prostate; seminal vesicles; vulva; bulbourethral glands; hair follicle; mesentery; pineal gland; subcutaneous tissue; veins; colon; hypothalamus; mammary glands; pituitary gland; teeth; vagina; cervix; interstitium; nose; parathyroid glands; tonsils; vas deferens; clitoris; kidneys; nails; anus; rectum; or testes.

In some embodiments, the biological test system is whole blood, or a cell from an embryo, e.g., a human embryo.

In some embodiments, a whole organism is used; the organism can be, e.g., a human, optionally a human subject in a clinical trial or a veterinary subject in a clinical trial, or a non-human model animal, e.g., a non-human mammal such as a mouse, rat, or rabbit, or can be a nematode, insect (e.g., drosophila), yeast, or bacterium.

### Interventions

The present methods can include applying one or more interventions to the test system. Interventions can include, for example, administration of one or more compounds, e.g., polypeptides, polynucleotides, or inorganic or organic large or small molecule test compounds. The intervention can also be, e.g., alteration of an environmental factor, e.g., food (e.g., quality or quantity of nutrition, calories, or type); exposure to toxic or potentially toxic environments (e.g., to mimic exposure to pollution or smoking); exposure to or administration of a therapeutic or potential therapeutic regimens, including genetic interventions, such as gene therapy (delivery of mRNAs and DNA into humans and animals with AAVs, liposomes, and so on) and genetic modification of human or animal cells ex vivo); exposure to increased or decreased oxygen levels; and so on. In some embodiments, the intervention comprises application of an agent that targets a transcript listed in Table A or a protein expressed from a transcript listed in Table A, e.g., a drug, antibody, or nucleic acid (e.g., an inhibitory nucleic acid such as an antisense oligonucleotide or siRNA) that targets a transcript listed in Table A or a protein expressed from a transcript listed in Table A, or a nucleic acid comprising an mRNA transcript listed in Table A. When more than one intervention is applied, the more than one can include multiple applications over time of the same intervention, or application of multiple interventions, e.g., at the same time or consecutively or over time.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. Natural compounds such as vitamins and neutraceuticals can also be tested using the present methods.

The top genes with the highest absolute weights in the clock models (Table A) can also be considered as targets for lifespan-extending and rejuvenative therapies. In addition to using tAge models to identify promising lifespan-extending and rejuventaive interventions, one can target top genes included in these models, using either genetic modification and gene therapy techniques, or small molecule compounds and biologicals (e.g., antibodies) that would bind to the products of these genes and affect their activity. For example, if the gene has a positive coefficient in the biological age clock model, then antibody targeting the product of this gene and inhibiting its activity would be a good candidate for rejuvenative and lifespan-extending treatment. The present methods can be used to evaluate such therapies.

### Methods for Determining Effects on Lifespan and Biological Age

The present methods include determining expression of one or more transcripts identified herein, i.e., in Table A. In some embodiments, the methods include determining expression of 5, 10, 20, 30, 40, 50, 60, 70, 80, 90, 100, 125, 150, or more transcripts described herein; in some embodiments, the methods include determining expression of up to 100, 125, 150, 175, 200, 250, 300, 350, 400, 500, or 1,000 transcripts, including at least 5, 10, 20, 30, 40, 50, 60, 70, 80, 90, 100, 125, 150, or all of the transcripts described herein. The methods can include applying an intervention to the system and determining expression of the one or more transcripts during and/or after application of the intervention.

As used herein, determining can include performing an assay (or causing an assay to be performed) on a test system to determine expression levels, or can include using existing expression data. Methods (assays) for determining expression of a specific transcript are known in the art, and include microarrays, RNA-seq, automated Sanger sequencing (e.g., using an ABI 3730x1 genome analyzer), pyrosequencing on a solid support (e.g., using 454 sequencing, Roche), sequencing-by-synthesis with reversible terminations (e.g., using an ILLUMINA® Genome Analyzer), sequencing-by-ligation (ABI SOLID®) or sequencing-by-synthesis with virtual terminators (HELISCOPE®); Moleculo sequencing (see Voskoboynik et al. eLife 2013 2: e00569 and U.S. patent application Ser. No. 13/608,778, filed Sep. 10, 2012); DNA nanoball sequencing; single molecule real time (SMRT) sequencing; Nanopore DNA sequencing; sequencing by hybridization; sequencing with mass spectrometry; and microfluidic Sanger sequencing. Exemplary next generation sequencing methods known to those of skill in the art include Massively parallel signature sequencing (MPSS), Polony sequencing, pyrosequencing (454), Illumina (Solexa) sequencing by synthesis, SOLID sequencing by ligation, Ion semiconductor sequencing (Ion Torrent sequencing), DNA nanoball sequencing, chain termination sequencing (Sanger sequencing), heliscope single molecule sequencing, single molecule real time (SMRT) sequencing (Pacific Biosciences); flow-based sequencing (e.g., Ultima sequencing) and nanopore sequencing such as is described at world wide website nanoporetech.com. Expression levels can be normalized using methods known in the art; for example, they can be normalized against expression of other genes: one or several housekeeping genes, if this is qRT-PCR; or the whole transcriptome, if microarray or RNA-seq is used. In the latter case, expression of every single gene is normalized relative to the expression of all ˜20,000 genes. Exemplary methods are provided herein in “Preprocessing of gene expression training data” and “Application of the clock model” in the Materials and methods section (e.g., RLE normalization, scaling, ranking, YoGene transformation, etc.).

An exemplary method can include some or all of the following steps:
1) Collection of tissue or cell material (e.g., piece of liver or brain or cell culture);
2) Extraction of RNA and library preparation;
3) RNA sequencing;
4) Mapping and counting of reads (output of sequencing);
5) Filtering, log-transformation and normalization of gene expression profiles;
6) Application of the clock model to the normalized gene expression profile by summing up products of gene normalized log-expression and corresponding weights from pre-trained Elastic Net linear model;
7) If log-log model is used, transformation of tAge as described in “Methods for determining effects on lifespan and biological age” can be performed; and
8) Comparison of average tAges in treated and control groups with t-tests, ANOVA, linear regression, or any other applicable statistical methods.

Where expression of a plurality (more than one) of transcripts is determined, an algorithm can be used to calculate expected total lifespan, expected remaining lifespan, chronological age, and/or lifespan-adjusted age, i.e., tAge, e.g., manual or software-based modeling algorithms such as a linear algorithms, e.g., elastic net analysis; a rank-based linear algorithm; principal component analysis (PCA); classification or decision trees; linear and polynomial support vector machines (SMV); shrunken centroids; random forest algorithms; support vector machines; gradient boosting; or neural networks.

For example, the methods can include calculating tAge using a preprocessed gene expression profile obtained from sequencing platforms or publicly available sources, e.g., databases. An exemplary method for determining tAge using a linear algorithm is as follows:

$$tAge = intercept + b 1 * T 1 + b 2 * T 2 + … + bn * Tn$$

Where b1-bn are the pre-trained model coefficients for every gene from Table A. and T1-Tn are the normalized expression levels of given Transcripts.

For log-log models (specified in Table A), tAge calculated from the above-mentioned formula, should be transformed as follows:

$${tAge}_{m} = 10 ** ( - 10 ** ( - tAge ) ) ,$$

where tAge is the estimate from the formula above, and tAgeₘ is a transformed tAge that reflects the chronological or lifespan-adjusted biological age on a scale of 0-1, where 0 is a moment of birth, and 1 is the maximum achievable lifespan for the particular model.

In differential clock models the resulting tAges reflect the relative difference in biological age between the samples, while other lifespan and aging clock models allow to assess expected lifespan, chronological age and lifespan-adjusted biological age in absolute terms.

In some embodiments, the methods include summing the product of expression level change and weight for each transcript, and determining if the sum is positive (i.e., protective) or negative (i.e., damaging). In some embodiments, the difference of the average tAge before and after the treatment is used.

In some embodiments, the methods include comparing an expression profile for one or more transcripts identified herein to a reference expression profile, or comparing a tAge determined in a test system with a reference tAge. The reference expression profile or tAge can be, e.g., a baseline expression profile or tAge obtained in the same test system, an expression profile or tAge obtained earlier in time in the same test system, or an expression profile or tAge in a reference system that represents the expression profile in the system in the absence of an intervention. The reference system is typically the same type as the test system (i.e., a matched control) and is as identical to the test system as possible.

A test compound can be identified as having a rejuvenative, healthspan-improving or lifespan-extending effect when changes in expression profile or tAge are observed that are consistent with protection as shown herein, i.e., reducing the chronological or lifespan-adjusted age predicted by tAge or increasing the predicted expected lifespan; conversely, a test compound can be identified as having a pro-aging or damaging effect on lifespan or health when changes in expression profile are observed that are consistent with damage as shown herein, i.e., increasing the chronological or lifespan-adjusted age predicted by tAge or reducing the predicted expected lifespan.

An intervention that has been screened by a method described herein and determined to have a positive effect on biological age, healthspan or lifespan (increased lifespan, reduced biological age) can be considered a candidate compound. A candidate compound that has been screened, e.g., in an in vivo model of a disorder such as a non-human test animal or a human subject in a clinical trial, and determined to have a positive effect on lifespan, mortality or health outcomes can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

The methods can also be used to identify interventions that are damaging, e.g., that can decrease lifespan or health outcomes or increase biological age or mortality; such interventions can be identified for avoidance or exclusion, e.g., in food, cosmetics, or pharmaceuticals.

Test compounds identified as protective hits can be considered candidate therapeutic compounds, useful in rejuvenation (e.g., slowing, delaying, or even reversing aging) and/or increasing lifespan. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test interventions identified as candidate protective interventions compounds can be further screened by administration to a test system in an animal model, e.g., as described herein. The animal can be monitored for a change in lifespan, mortality and/or for change (preferably an improvement) in a parameter of aging, e.g., a parameter related to health or clinical outcome. In some embodiments, the parameter is development or progression of age-related conditions such as hearing loss, cataracts and refractive errors, back and neck pain and osteoarthritis, chronic obstructive pulmonary disease, diabetes, and dementia, and an improvement would be a delay or decrease in risk of development of one or more age-related conditions. In some embodiments, the test system is epidermis, and the parameter is development or progression of age-related skin conditions such as thinning, sagging, wrinkling, xerosis, pruritis, eczematic dermatitis, purpura, and chronic venous insufficiency, and an improvement would be a delay or decrease in risk of development of one or more age-related conditions.

Standard computing devices and systems can be used and implemented to perform the methods described herein. Computing devices include various forms of digital computers, such as laptops, desktops, mobile devices, workstations, personal digital assistants, servers, blade servers, mainframes, and other appropriate computers. In some embodiments, the computing device is a mobile device, such as personal digital assistant, cellular telephone, smartphone, tablet, or other similar computing device. The components described herein, their connections and relationships, and their functions, are meant to be exemplary only, and are not meant to limit implementations of the inventions described and/or claimed in this document.

Computing devices typically include one or more of a processor, memory, a storage device, a high-speed interface connecting to memory and high-speed expansion ports, and a low-speed interface connecting to low speed bus and storage device. Each of the components are interconnected using various busses, and can be mounted on a common motherboard or in other manners as appropriate. The processor can process instructions for execution within the computing device, including instructions stored in the memory or on the storage device to display graphical information for a GUI on an external input/output device, such as a display coupled to a high-speed interface. In other implementations, multiple processors and/or multiple buses can be used, as appropriate, along with multiple memories and types of memory. Also, multiple computing devices can be connected, with each device providing portions of the operations (e.g., as a server bank, a group of blade servers, or a multi-processor system).

FIG. 8 shows an example computer system 500 that includes a processor 510, a memory 520, a storage device 530 and an input/output device 540. Each of the components 510, 520, 530 and 540 can be interconnected, for example, by a system bus 550. The processor 510 is capable of processing instructions for execution within the system 500. In some implementations, the processor 510 is a single-threaded processor, a multi-threaded processor, or another type of processor. The processor 510 is capable of processing instructions stored in the memory 520 or on the storage device 530. The memory 520 and the storage device 530 can store information within the system 500.

The input/output device 540 provides input/output operations for the system 500. In some implementations, the input/output device 540 can include one or more of a network interface device, for example, an Ethernet card, a serial communication device, for example, an RS-232 port, or a wireless interface device, for example, an 802.11 card, a 3G wireless modem, a 4G wireless modem, or a 5G wireless modem, or both. In some implementations, the input/output device can include driver devices configured to receive input data and send output data to other input/output devices, for example, keyboard, printer and display devices 560. In some implementations, mobile computing devices, mobile communication devices, and other devices can be used. In some embodiments, the present methods are performed using a device comprising a sequencing machine, e.g., an Illumina sequencer.

TABLE A

| | | | | | | Chronological | | Lifespan- |
|---|---|---|---|---|---|---|---|---|
| | | | | | Chronological | age multi- | Lifespan- | adjusted age |
| | | Chronological | Chronological | Chronological | age multi- | tissue | adjusted age | multi-tissue |
| Entrez | Gene | age liver | age brain | age muscle | tissue mouse | mouse + rat | liver clock | mouse clock |
| ID | symbol | clock (log-log) | clock (log-log) | clock (log-log) | clock (log-log) | clock (log-log) | (log-log) | (log-log) |
| 12457 | Noct | 0.010816754 | 0 | 0 | 0.012165034 | 0.003815409 | 0.009009806 | 0.005846997 |
| 20860 | Sult1e1 | 0.007665358 | 0 | 0 | 0.006551794 | 0.00263413 | 0.007189779 | 0 |
| 331535 | Serpina7 | 0.007430592 | 0 | 0 | 0 | 0.002914869 | 0.007044329 | 0 |
| 17381 | Mmp12 | 0.006607875 | 0 | 0 | 0.006986494 | 0.004488086 | 0.005402245 | 0.00390609 |
| 232174 | Cyp26b1 | 0.00618786 | 0 | 0 | 0.006380743 | 0.002712204 | 0.003755663 | 0.004171031 |
| 319476 | Lrtm1 | 0.005973104 | 0 | 0 | 0.006053879 | 0.002778242 | 0.004370849 | 0.00372206 |
| 16008 | Igfbp2 | 0.005899564 | 0 | 0 | 0.004676685 | 0.00306437 | 0.001893692 | 0.001147202 |
| 13082 | Cyp26a1 | 0.005680829 | 0 | 0 | 0.001065711 | 0.001792864 | 0.009099146 | 0 |
| 109901 | Cela1 | −0.00565649 | 0 | 0 | 0.0011449 | −0.00114461 | −0.00472812 | 0.000245481 |
| 16819 | Lcn2 | 0.005621133 | 0 | 0 | 0.003884344 | 0.003300399 | 0.006112821 | 0.007904002 |
| 12550 | Cdh1 | −0.00549596 | 0.025763052 | 0 | −0.00345552 | −0.00132455 | −0.00492466 | −0.00221856 |
| 66222 | Serpinb1a | 0.005364965 | 0 | 0 | 0.004303996 | 0.002174631 | 0.005707333 | 0.003622376 |
| 16006 | Igfbp1 | −0.00534461 | 0 | 0 | −0.00580583 | −0.002201 | −0.00686522 | 0 |
| 11997 | Akr1b7 | 0.005287662 | 0 | 0 | 0 | 0.002299979 | 0.004315513 | 0 |
| 17748 | Mt1 | −0.00512669 | 0 | 0 | −0.00339933 | −0.00137048 | −0.00629349 | −0.00278774 |
| 57429 | Sult5al | −0.0050886 | 0 | 0 | −0.00522955 | −0.00218486 | −0.00275271 | −0.00121745 |
| 20201 | S100a8 | 0.005069559 | 0 | 0 | 0.009335328 | 0.004783994 | 0.005718452 | 0 |
| 68680 | Fitm1 | 0.005032374 | 0 | 0 | 0.004272237 | 0.001935584 | 0.004017508 | 0.001813842 |
| 93695 | Gpnmb | 0.005010102 | 0.117447012 | 0.01148035 | 0.01353898 | 0.009182415 | 0.006082749 | 0.009857972 |
| 94226 | S1pr5 | 0.004920529 | 0 | 0 | 0.003773977 | 0.001987347 | 0.004910655 | 0.002254162 |
| 16069 | Jchain | 0.004835609 | 0 | 0.013102004 | 0.008405863 | 0.008137126 | 0.006198433 | 0.011359555 |
| 27276 | Plekhb1 | 0.004826893 | 0 | 0 | 0.003888124 | 0.003172862 | 0.005391156 | 0.00436957 |
| 13119 | Cyp4a14 | 0.004757258 | 0 | 0 | 0.002938477 | 0.001057928 | 0.003541409 | 0 |
| 21987 | Tpd52l1 | 0.004688226 | 0 | 0 | 0.00335955 | 0.003235169 | 0.004280822 | 0.002208786 |
| 116939 | Pnpla3 | 0.004658206 | 0 | 0 | 0.003266429 | 0.001406092 | 0.004996298 | 0.001814925 |
| 16854 | Lgals3 | 0.004588154 | 0.018797866 | 0 | 0.00858355 | 0.00462651 | 0.004365802 | 0.00630214 |
| 71690 | Esm1 | 0.00457672 | 0 | 0 | −6.95E−05 | 0.001573174 | 0.004699227 | 0.000427707 |
| 66438 | Hamp2 | −0.00455729 | 0 | 0 | 0 | −0.00156227 | −0.00271354 | 0 |
| 11833 | Aqp8 | −0.00452842 | 0 | 0 | −0.00317106 | 5.19E−05 | −0.00149585 | −0.00042876 |
| 51902 | Rnf24 | −0.00446177 | 0 | 0 | −0.00306633 | −0.00113735 | −0.00220181 | −0.00116699 |
| 21743 | Inmt | −0.00445599 | 0 | 0 | −0.00280687 | −0.00145543 | −0.00631705 | −0.00275991 |
| 17436 | Me1 | −0.00443935 | 0 | 0 | −0.00102541 | −0.00045852 | −0.00413537 | −0.00060304 |
| 19116 | Prlr | 0.00435082 | 0 | 0 | −0.00036026 | 0.001582664 | 0.006576957 | 0.002509339 |
| 14945 | Gzmk | 0.004350231 | 0 | 0 | 0 | 0.003457358 | 0.003456301 | 0 |
| 20775 | Sqle | 0.004323303 | 0 | 0 | 0.004130236 | 0.001609848 | 0.002519143 | −0.00015935 |
| 13370 | Dio1 | −0.00430204 | 0 | 0 | −0.00440194 | −0.00292462 | −0.00395974 | 0 |
| 19879 | Slc22a8 | −0.00430094 | 0 | 0 | 0.000494444 | −0.00211645 | −0.00472688 | 0 |
| 27409 | Abcg5 | −0.00429813 | 0 | 0 | 0.001902603 | −0.00158159 | −0.00483648 | 0.000260102 |
| 14825 | Cxcl1 | 0.004219227 | 0 | 0 | 0.002254635 | 0.003212088 | 0.002559582 | 0 |
| 20304 | Ccl5 | 0.004209594 | 0 | 0 | 0.003958188 | 0.002954603 | 0.003462754 | 0.000860883 |
| 100043194 | Sult2a2 | −0.00418163 | 0 | 0 | 0 | 0 | −0.00482422 | 0 |
| 246277 | Csad | −0.00416508 | 0 | 0 | −0.00091946 | −0.00068822 | −0.00232135 | −0.00086686 |
| 72002 | Slc39a5 | −0.00414591 | 0 | 0 | −0.00288623 | −0.00144409 | −0.00454422 | 0 |
| 211187 | Lrtm2 | 0.004145495 | 0 | 0 | −0.00059312 | 0.00069312 | 0.002374994 | 0 |
| 93671 | Cd163 | −0.00412538 | 0 | 0 | −0.00556065 | −0.00020231 | −0.00385389 | −0.00231748 |
| 13653 | Egr1 | 0.004108094 | 0 | 0.011398377 | 0.001367703 | 0.002238218 | 0.003259245 | 0.001291373 |
| 28248 | Slco1a1 | −0.00409462 | 0 | 0 | 0 | −0.00040785 | −0.00390156 | 0 |
| 242509 | Bnc2 | 0.004093987 | 0 | 0 | 0.003316856 | 0.001826642 | 0.002732553 | 0.00117902 |
| 114886 | Cygb | 0.004048481 | 0 | 0 | 0.004641557 | 0.002712883 | 0.003919837 | 0.002938172 |
| 231691 | Sds | −0.00403253 | 0 | 0 | 0.000511363 | −0.00230841 | −0.00310318 | 0 |
| 70237 | Bhlhb9 | −0.00398082 | 0 | 0 | −0.00237503 | −0.00169197 | −0.00425512 | −0.00137504 |
| 69772 | Bdh2 | 0.003952219 | 0 | 0 | 0.003293012 | 0.00138173 | 0.003543554 | 0.002330685 |
| 14859 | Gsta3 | 0.003943216 | 0 | 0 | 0.003598536 | 0.00362607 | 0.003643791 | 0.0022792 |
| 13099 | Cyp2c40 | −0.00393569 | 0 | 0 | 0 | 0 | −0.00253511 | 0 |
| 216831 | Arhgap44 | −0.00391389 | 0 | 0 | −0.00178573 | −0.00074546 | −0.00209252 | 0.001073545 |
| 76560 | Prss8 | 0.003911469 | 0 | 0 | 0.000466272 | 0.002136033 | 0.004140102 | 0.001044038 |
| 12832 | Col5a2 | −0.00389799 | 0 | 0 | −0.00767163 | −0.00398493 | −0.00237368 | −0.00490308 |
| 20210 | Saa3 | 0.003880559 | 0 | 0 | 0 | 0.002434512 | 0.001629038 | 0 |
| 20200 | S100a6 | 0.003848114 | 0.008515953 | 0 | 0.008373422 | 0.003458171 | 0.002368876 | 0.003549856 |
| 654812 | Angptl7 | −0.00383331 | 0 | 0 | −0.00391899 | −0.00147331 | −0.00131773 | −0.00126389 |
| 319996 | Golm2 | −0.00382008 | 0 | 0 | −0.00399255 | −0.00209966 | −0.00275889 | −0.00204459 |
| 26875 | Pclo | 0.00378431 | 0 | 0 | 0.005344197 | 0.002736149 | 0.003323919 | 0.003237255 |
| 75772 | Pnpla5 | 0.003741082 | 0 | 0 | 0 | 0.001927595 | 0.003492442 | 0 |
| 16840 | Cnmd | 0.003739732 | 0 | 0 | 0.003847878 | 0.000743023 | 0.002883752 | 0 |
| 22127 | Tsx | 0.003739333 | 0 | 0 | 0 | 0.001453397 | 0.003994632 | 0 |
| 56744 | Pf4 | 0.00373257 | 0 | 0 | 0.001917766 | 0.001050099 | 0.003243213 | 0.000267777 |
| 211949 | Spsb4 | 0.003687987 | 0 | 0 | 0.001100242 | 0.000658036 | 0.004009932 | 0.000493031 |
| 140577 | Ankrd6 | 0.003687659 | 0 | 0 | 0.00198713 | 0.001855188 | 0.003174078 | 0.001857524 |
| 14803 | Grid1 | 0.003687178 | 0 | 0 | 0.003861524 | 0.001725318 | 0.003740105 | 0 |
| 230590 | Zyg11a | −0.00366659 | 0 | 0 | −0.00367904 | −0.00175382 | −0.00277748 | 0 |
| 105349 | Akr1c18 | −0.00365605 | 0 | 0 | 0 | −0.00236878 | −0.00290509 | 0 |
| 13117 | Cyp4a10 | −0.00365582 | 0 | 0 | 0 | 0 | −0.00075557 | 0 |
| 14137 | Fdft1 | 0.003629811 | 0 | 0 | 0.001742865 | 0.001103418 | 0.002691548 | 0.000158113 |
| 17387 | Mmp14 | 0.003621205 | 0 | 0 | 0.005315147 | 0.001989111 | 0.003161923 | 0.003570901 |
| 84112 | Sucnr1 | 0.003612491 | 0 | 0 | 0.000312278 | 0.000803232 | 0.003300235 | 0.001253662 |
| 68396 | Nat8 | 0.003607727 | 0 | 0 | 0.002235291 | 0.00069241 | 0.003582156 | 0 |
| 242523 | Dmrta1 | 0.003586619 | 0 | 0 | 0 | 0.001753111 | 0.004524213 | 0 |
| 59012 | Moxd1 | −0.00357789 | 0.00180552 | 0 | −0.00129961 | 2.68E−05 | −0.005329 | −0.00181844 |
| 20198 | S100a4 | 0.00357507 | 0.001607185 | 0 | 0.00389423 | 0.003016306 | 0.002335711 | 0.002985285 |
| 93694 | Clec2d | −0.00354096 | 0 | 0 | −0.0072003 | −0.00274357 | −0.00285175 | 0 |
| 11611 | Agxt | 0.003498531 | 0 | 0 | 0 | 0.00044072 | 0.002471283 | 0 |
| 171405 | Slc22a27 | 0.003495582 | 0 | 0 | 0 | 0 | 0.001407141 | 0 |
| 56636 | Fgf21 | 0.003493779 | 0 | 0 | 0.003318225 | 0.001963185 | 0.001313949 | 0 |
| 57435 | Plin4 | −0.00345036 | 0 | 0 | −0.00332803 | −0.00107253 | −0.00167276 | −0.00093108 |
| 246691 | Prok1 | 0.003427474 | 0 | 0 | 0 | 0 | 0.002850418 | 0 |
| 192156 | Mvd | 0.003398839 | 0 | 0 | 0.002758708 | 0.000459713 | 0.003228116 | 0.00036557 |
| 75487 | Ppp1r3g | −0.00339043 | 0 | 0 | −0.0022116 | −0.000305 | −0.00024181 | 0 |
| 140904 | Caln1 | 0.00337966 | 0 | 0 | −0.00035664 | 0.001374831 | 0.003682106 | 0 |
| 52668 | Ifi27 | 0.003375548 | 0 | 0 | 0.005196429 | 0.002026312 | 0.00389125 | 0.002754933 |
| 15496 | Hsd3b5 | −0.00337522 | 0 | 0 | 0 | 0 | −0.0012018 | 0 |
| 16147 | Ihh | 0.003366874 | 0 | 0 | 0.005336493 | 0.002138962 | 0.001941289 | 0.001066233 |
| 21413 | Tcf4 | 0.003355856 | 0 | 0 | 0.002284472 | 0.00100219 | 0.002628625 | 0.001259841 |
| 27528 | Nrep | −0.00335436 | −0.02378986 | −0.02367302 | −0.00419095 | −0.00725583 | −0.00445624 | −0.00629414 |
| 114332 | Lyve1 | −0.0033525 | 0.004877457 | 0 | −0.00324545 | −0.00110102 | −0.00298585 | −0.00177552 |
| 624219 | Angptl8 | 0.003344094 | 0 | 0 | 0.001349294 | 0.001197617 | 0.003158313 | −0.00139753 |
| 69060 | Pnlip | −0.00334357 | 0 | 0 | 0 | −0.00083456 | −0.00330219 | 0 |
| 237611 | Stac3 | −0.00333054 | 0 | 0 | −0.00022827 | −0.00255057 | −0.00479419 | −0.00072278 |
| 17060 | Blnk | 0.003317258 | 0 | 0 | 0.00315247 | 0.001590049 | 0.003198467 | 0.002050356 |
| 67470 | Abcg8 | −0.00330531 | 0 | 0 | 0.001448045 | −0.00131685 | −0.00310121 | 0 |
| 12350 | Car3 | −0.00327915 | 0 | 0.002508462 | −0.00305593 | −0.00166977 | −0.00450781 | −0.00223853 |
| 18407 | Orm3 | 0.003270484 | 0 | 0 | 0.00342317 | 0.000559541 | 0.004062389 | 0 |
| 232400 | A2ml1 | 0.003250901 | 0 | 0 | 0.001290271 | 0.002167099 | 0.003130976 | 0 |
| 243755 | Slc13a4 | −0.00324801 | 0 | 0 | −0.00184468 | −0.00237117 | −0.00345906 | −0.00155298 |
| 19152 | Prtn3 | 0.003234204 | 0 | 0 | 0.003528578 | 0.001763137 | 0.003420784 | 0 |
| 76703 | Cpb1 | −0.00322292 | 0 | 0 | −0.0028374 | −0.00202387 | −0.0026451 | 0 |
| 234199 | Fgl1 | 0.003211346 | 0 | 0 | 0.00164562 | 0.001440595 | 0.003411805 | 0 |
| 15122 | Hba-a1 | −0.00320754 | 0 | 0 | −0.00511321 | −0.00230742 | −0.00335088 | −0.00375151 |
| 100034251 | Wfdc17 | 0.003204658 | 0 | 0 | 0.007737537 | 0.003136767 | 0.003670334 | 0.004372696 |
| 213649 | Arhgef19 | 0.003200333 | 0 | 0 | 0.003437877 | 0.002136889 | 0.003084594 | 0.002912657 |
| 17750 | Mt2 | −0.00318345 | 0 | 0 | 0.000195857 | −0.00111934 | −0.00378587 | −0.00049278 |
| 22242 | Umod | −0.00318119 | 0 | 0 | 0 | −0.00093322 | −0.00256289 | 0 |
| 117586 | A1bg | 0.003176282 | 0 | 0 | 0 | 0 | 0.005331609 | 0 |
| 20463 | Cox7a2l | 0.003173621 | 0 | 0 | 0.003866827 | 0.001294348 | 0.001806995 | 0.00156032 |
| 66684 | Tceal8 | −0.00316834 | 0 | 0 | −0.00382669 | −0.00054647 | −0.00316258 | −0.00170536 |
| 72297 | B3gnt3 | 0.003154624 | 0 | 0 | 0.002959672 | 0.000792519 | 0.00184835 | 0.000741743 |
| 54613 | St3gal6 | 0.003137522 | 0 | 0 | 0.003234166 | 0.001019554 | 0.003494476 | 0.002658114 |
| 94179 | Krt23 | −0.0031362 | 0 | 0 | −0.0013003 | −0.00140307 | −0.00364787 | −0.00272821 |
| 55985 | Cxcl13 | −0.00313464 | 0 | 0.020253563 | 0.009790374 | 0.00468117 | −0.00218333 | 0.006172906 |
| 57742 | Abhd1 | −0.00313125 | 0 | 0 | −0.00608781 | −0.00211934 | −0.00196718 | −0.00313073 |
| 319317 | Snhg11 | 0.003119143 | 0 | 0 | 0.005534129 | 0.003261462 | 0.001913672 | 0.002709006 |
| 54123 | Irf7 | 0.003107551 | 0 | 0.00732674 | 0.004796045 | 0.002847988 | 0.002664992 | 0.00313616 |
| 15985 | Cd79b | −0.00307418 | 0 | 0 | −0.00456396 | −0.00158071 | −0.00130214 | −4.94E−05 |
| 66184 | Rps4l | −0.00307321 | 0 | 0 | −0.00379323 | −0.002154 | −0.00278853 | −0.00161375 |
| 75178 | Meiob | −0.00306943 | 0 | 0 | 0 | −0.00027743 | −0.00250125 | 0 |
| 53614 | Reck | −0.00306501 | 0 | 0 | −0.00302434 | −0.00053238 | −0.00309176 | −0.00093667 |
| 13105 | Cyp2d9 | −0.00306218 | 0 | 0 | −0.00469724 | −0.00179344 | −0.00371114 | 0 |
| 11529 | Adh7 | 0.00305895 | 0 | 0 | 0.001224361 | 0.000962415 | 0.000606736 | 7.82E−05 |
| 214523 | Tmprss4 | −0.00303877 | 0 | 0 | −0.00323087 | −0.00106192 | −0.00032494 | 0 |
| 235135 | Tmem45b | 0.003018545 | 0 | 0 | 0.003829412 | 0.001015437 | 0.003090132 | 0.000459649 |
| 21786 | Tff3 | 0.003014855 | 0 | 0 | 0 | 0.001469406 | 0.000484555 | 0 |
| 21673 | Dntt | 0.00300298 | 0 | 0 | 0.002505416 | 0.000546822 | 0.003075314 | 0 |
| 75104 | Mmd2 | 0.002998737 | 0 | 0 | 0.004144181 | 0.000890318 | 0.001669157 | 0.001652576 |
| 245527 | Eda2r | 0.002998436 | 0 | 0 | 0.004622548 | 0.00279575 | 0.002312304 | 0.003392056 |
| 14373 | G0s2 | 0.002979303 | 0 | 0 | 0.005268467 | 0.002380298 | 0.001127306 | 0.0024982 |
| 13116 | Cyp46a1 | 0.002968088 | 0 | 0 | 0.004858936 | 0.001947508 | 0.001764816 | 0.002313871 |
| 16841 | Lect2 | −0.00296683 | 0 | 0 | −0.00020246 | −0.00057761 | −0.00335094 | 0 |
| 20592 | Kdm5d | 0.002959611 | 0 | 0 | 0.001904244 | 0.001695823 | 0.002939332 | 0.003225263 |
| 22349 | Vil1 | 0.002952244 | 0 | 0 | 0.008629041 | 0.002747859 | 0.002373797 | 0 |
| 109660 | Ctrl | −0.00294991 | 0 | 0 | 0 | −0.00099624 | −0.00257557 | 0 |
| 193740 | Hspa1a | −0.00294213 | 0 | 0 | −0.00291037 | −0.00131719 | −0.00307564 | 0 |
| 12825 | Col3a1 | −0.00290412 | −0.02926313 | −0.02191129 | −0.00702147 | −0.00675149 | −0.00296599 | −0.00745559 |
| 12482 | Ms4a1 | 0.002900554 | 0 | 0 | 0.008597989 | 0.003314603 | 0.002520448 | 0.004280177 |
| 58176 | Rhbg | −0.00288971 | 0 | 0 | −0.00082417 | −0.00096352 | −0.0035138 | −0.0007742 |
| 67701 | Wfdc2 | 0.002888548 | 0 | 0 | 0.00339545 | 0.002261158 | 0.005457876 | 0.004772535 |
| 26877 | B3galt1 | 0.002885379 | 0 | 0 | 0.002501664 | 0.001215476 | 0.002135582 | 0.002414868 |
| 15139 | Hc | 0.002877987 | 0 | 0 | 0.001572566 | 0.00134019 | 0.001221411 | −0.00024155 |
| 94185 | Tnfrsf21 | 0.002873094 | 0 | 0 | −0.00157563 | −9.90E−05 | 0.003232641 | −0.00015918 |
| 26900 | Ddx3y | 0.002847745 | 0.004158075 | 0.000308639 | 0.002337491 | 0.002156734 | 0.003081353 | 0.003845009 |
| 12613 | Cel | −0.00284651 | 0 | 0 | 0 | −0.00089839 | −0.00198056 | 0 |
| 16847 | Lepr | −0.00284411 | 0 | 0 | −0.00133981 | −0.00083613 | −0.00026299 | 0.000627191 |
| 18414 | Osmr | 0.0028427 | 0 | 0 | 0.005029351 | 0.002977323 | 0.002302242 | 0.003791974 |
| 17841 | Mup2 | −0.00284148 | 0 | 0 | 0 | 0 | −0.00291079 | 0 |
| 12934 | Dpysl2 | 0.00283609 | 0 | 0 | 0.001078513 | 0.00133764 | 0.00278507 | 0.001458863 |
| 12332 | Capg | 0.00282721 | 0 | 0 | 0.004125146 | 0.002059125 | 0.002437962 | 0.002770941 |
| 105348 | Golm1 | −0.00282528 | 0 | 0 | −0.00279061 | −0.00090702 | −0.00120112 | −0.00051479 |
| 53973 | Cyp3a41a | −0.0028246 | 0 | 0 | 0 | 0 | −0.00049141 | 0 |
| 72310 | Nkg7 | 0.002824323 | 0 | 0 | 0.000993183 | 0.002355754 | 0.002044809 | 0.000484094 |
| 71461 | Ptk7 | −0.00282253 | 0 | 0 | −0.0035022 | −0.00220386 | −0.00175085 | −0.00284685 |
| 228966 | Ppp1r3d | −0.00281509 | 0 | 0 | −0.00163101 | −7.36E−05 | 0 | 0.000471481 |
| 94353 | Hmgn3 | 0.00281466 | 0 | 0 | 0.002101755 | 0.000904139 | 0.001952265 | −7.96E−05 |
| 109821 | F11 | −0.00281126 | 0 | 0 | −0.0025558 | −0.0010472 | −0.00172345 | 0 |
| 226041 | Pgm5 | 0.002803618 | 0 | 0 | 0.003486333 | 0.00137103 | 0.00110165 | 0.001710895 |
| 11717 | Ampd3 | 0.002802762 | 0 | 0 | 0.004538829 | 0.002901939 | 0.001463293 | 0.002719901 |
| 26876 | Adh4 | 0.002797299 | 0 | 0 | 0 | 0 | 0.0009733 | 0 |
| 213742 | Xist | −0.00279455 | 0 | 0 | −0.00254438 | −0.00218156 | −0.00066217 | −0.00019328 |
| 17189 | Mb | −0.00278236 | 0 | 0 | −0.00302061 | −0.00142048 | −0.00275315 | −0.00218196 |
| 11472 | Actn2 | −0.00277948 | 0 | 0 | −0.00219655 | −0.00109764 | −0.00190521 | −0.00165376 |
| 76982 | Vxn | 0.002773619 | 0 | 0 | 0.000727084 | 0.00122812 | 0.002618255 | 0 |
| 74182 | Gpcpd1 | −0.00277039 | 0 | 0 | −0.00252102 | −0.00216563 | −0.0015833 | −0.00183642 |
| 230822 | Ncmap | 0.002759178 | 0 | 0 | 0 | 0.000687146 | 0.002072619 | 0 |
| 50914 | Olig1 | 0.00275869 | 0 | 0 | 0.003482373 | 0.001454657 | 0.002277968 | 0 |
| 73690 | Glipr1 | 0.002751406 | 0 | 0 | 0.002259092 | 0.002205832 | 0.001839147 | 0 |
| 16643 | Klrd1 | 0.002748911 | 0 | 0 | 0.001639588 | 0.001890656 | 0.002338847 | 0 |
| 14977 | Slc39a7 | 0.002746645 | 0 | 0 | 0.001479757 | 0.000276878 | 0.001789579 | 0.000282145 |
| 56727 | Miox | −0.00274439 | 0 | 0 | 0 | −0.00188122 | −0.00213022 | 0 |
| 620807 | Mup6 | −0.00273631 | 0 | 0 | 0 | 0 | −0.00234384 | 0 |
| 59056 | Evc | −0.00273526 | 0 | 0 | −0.00111982 | 0.000341386 | −0.00074987 | 0.000746486 |
| 12525 | Cd8a | 0.002726985 | 0 | 0 | 0.000670839 | 0.001471331 | 0.001665936 | 0 |
| 15894 | Icam1 | 0.002724261 | 0 | 0 | 0.004460326 | 0.002694783 | 0.001978391 | 0.001775924 |
| 66180 | P3h4 | −0.00270805 | 0 | 0 | −0.00240406 | −0.00075716 | −0.00201753 | −0.00074586 |
| 57738 | Slc15a2 | −0.00270629 | 0 | 0 | −0.00278833 | −0.0001676 | −0.00109153 | 4.90E−05 |
| 83433 | Trem2 | 0.002706009 | 0 | 0 | 0.005898182 | 0.003369533 | 0.001329995 | 0.003899125 |
| 13713 | Elk3 | 0.002704629 | 0 | 0 | 0.00179349 | 0.00074821 | 0.002476144 | 0.001012428 |
| 231510 | Gpat3 | −0.00269472 | 0 | 0 | −0.00195 | −0.00073771 | −0.00222753 | −0.00070703 |
| 15490 | Hsd17b7 | 0.002692691 | 0.01148997 | 0 | 0.004227478 | 0.001911329 | 0.002088576 | 0.001306193 |
| 60599 | Trp53inp1 | −0.00269062 | 0 | 0 | −0.0019911 | −0.00054175 | −0.00101594 | −0.00084949 |
| 17897 | Myl3 | −0.00268386 | 0 | 0 | −0.00401394 | −0.00192301 | −0.00203552 | 0 |
| 239037 | Lrit1 | −0.00268327 | 0 | 0 | −0.00372208 | −0.00078429 | −0.00174641 | 0 |
| 105727 | Slc38a1 | −0.00268181 | 0 | 0 | −0.00675819 | −0.00354175 | −0.00216851 | −0.00482493 |
| 55193 | Plek | 0.002679626 | 0 | 0 | 0.004485856 | 0.002615449 | 0.002611075 | 0.003120338 |
| 56485 | Slc2a5 | 0.002677364 | 0 | 0 | −0.00035228 | 0.001025063 | 0.002359057 | −0.00075742 |
| 13197 | Gadd45a | 0.002676661 | 0 | 0.031973728 | 0.001152732 | 0.001975114 | 0.002124536 | 0.001038395 |
| 241447 | Cers6 | −0.00266952 | 0 | 0 | −0.00277682 | −0.00189074 | −0.00151513 | −0.00052536 |
| 16323 | Inhba | 0.002654604 | 0 | 0 | 0.003777421 | 0.002258423 | 0.000884627 | 0.002044174 |
| 14056 | Ezh2 | −0.00265241 | 0 | 0 | −0.00479664 | −0.00232557 | −0.00248428 | −0.00359001 |
| 13041 | Ctsw | 0.002652266 | 0 | 0 | 0.002481656 | 0.001327673 | 0.001116499 | 0.000171223 |
| 16427 | Itih4 | 0.002650189 | 0 | 0 | −0.00361111 | −0.00053182 | 0.001837745 | −0.00129795 |
| 13121 | Cyp51 | 0.002649314 | 0 | 0 | 4.12E−05 | −4.27E−05 | 0.001746549 | −0.00164837 |
| 328977 | Zfp532 | −0.00264309 | 0 | 0 | −0.00402245 | −0.00161373 | −0.00229326 | −0.00268857 |
| 319554 | Idi1 | 0.002642615 | 0 | 0 | −0.00104106 | −0.00032698 | 0.002309304 | −0.00054155 |
| 276891 | Timd4 | −0.00263505 | 0 | 0 | −0.00605335 | −0.00188187 | −0.00258279 | −0.00307405 |
| 19734 | Rgs16 | 0.00263409 | 0 | 0 | −0.00044445 | 0.001878706 | 0.0009642 | −0.00146226 |
| 11464 | Actc1 | −0.00263371 | 0 | 0 | −0.0043832 | −0.00188671 | −0.0014657 | −0.00269415 |
| 112407 | Egln3 | 0.002631752 | 0 | 0 | 0.003375908 | 0.002295011 | 0.001892133 | 0.002958212 |
| 230899 | Nppa | −0.0026315 | 0 | 0 | −0.00202876 | −0.0006121 | −0.00170671 | 0 |
| 171285 | Havcr2 | 0.002627233 | 0 | 0 | 0.00145879 | 0.001161185 | 0.00153833 | 0.001508568 |
| 20392 | Sgce | −0.00262635 | 0 | 0 | −0.0015484 | −0.00087402 | −0.00157877 | −0.00095861 |
| 25464 | Vnn3 | −0.00262552 | 0 | 0 | −0.00191602 | −0.00096328 | −0.00156578 | −0.00124629 |
| 19215 | Ptgds | −0.0026209 | 0 | 0 | −0.00253616 | 0.000185958 | −0.00210452 | −0.00093485 |
| 14027 | Evpl | 0.002620227 | 0 | 0 | 0.003904721 | 0.001528325 | 0.000684466 | 0.001443634 |
| 12053 | Bcl5 | −0.00261947 | 0 | 0 | 0.000258514 | −0.0008375 | −0.00212657 | −0.00077632 |
| 230558 | C8a | −0.00261597 | 0 | 0 | −0.00272699 | −0.00115988 | −0.00137263 | 0 |
| 13649 | Egfr | −0.00261582 | 0 | 0 | −0.00330312 | −0.00210249 | −0.0030265 | −0.00147133 |
| 74020 | Cpne4 | 0.002611115 | 0.0320461 | 0 | 0.002674376 | 0.002915465 | 0.002762441 | 0 |
| 241633 | Atp8b4 | 0.002610643 | 0 | 0 | 0.003332857 | 0.001335504 | 0.002038037 | 0.000819318 |
| 22758 | Zscan12 | 0.00261016 | 0 | 0 | 0.004459058 | 0.001762345 | 0.001755976 | 0.002163006 |
| 26357 | Abcg2 | −0.00260712 | 0 | 0 | −0.00144655 | −0.00090978 | −0.00290819 | −0.00074502 |
| 227231 | Cps1 | 0.002607072 | 0 | 0 | 0.005616464 | 0.000229775 | 0.001097203 | 0.000654687 |
| 231633 | Tmem119 | −0.00259145 | 0 | 0 | −0.00056323 | −0.00135359 | −0.00311208 | −0.00247197 |
| 73673 | Rec114 | −0.00257922 | 0 | 0 | 0 | −1.23E−05 | −0.00091163 | 0 |
| 12512 | Cd63 | 0.002571429 | 0 | 0 | 0.005438439 | 0.002581176 | 0.001710535 | 0.002868709 |
| 70676 | Gulp1 | 0.002569193 | 0 | 0 | 0.002239132 | 0.000280833 | 0.002033222 | 0.000408765 |
| 21408 | Zfp354a | −0.00256715 | 0 | 0 | −0.00109891 | −0.00145874 | −0.00200989 | −0.00091593 |
| 20537 | Slc5a1 | 0.002567026 | 0 | 0 | 0.004171 | 0.000872093 | 0.002821682 | 0.002655902 |
| 14685 | Gnat1 | 0.002555728 | 0 | 0 | 0.000671231 | 0.000366268 | 0.002909095 | 0 |
| 66433 | Chchd7 | −0.00255542 | 0 | 0 | 0.000270567 | −7.29E−05 | −0.00292167 | 0.000149375 |
| 26362 | Axl | 0.002555367 | 0 | 0 | 0.00245291 | 0.001083638 | 0.00263904 | 0.002195749 |
| 14734 | Gpc3 | 0.002555103 | 0 | 0 | 0.005359984 | 0.001334425 | 0.001962168 | 0.002134124 |
| 109225 | Ms4a7 | 0.002554195 | 0 | 0 | 0.004811172 | 0.003174212 | 0.001955252 | 0.004533146 |
| 104910 | Slc25a47 | 0.002549215 | 0 | 0 | 0.001044697 | −3.60E−05 | 0.003147402 | 0.000163036 |
| 214854 | Neurl3 | 0.002541643 | 0 | 0 | −0.00384278 | 0.000228994 | 0.003076745 | −0.00225772 |
| 26358 | Aldh1a7 | 0.00254126 | 0 | 0 | 0.003447693 | 0.001204133 | 0.002189212 | −0.00083826 |
| 625098 | Slc38a6 | −0.002541 | 0 | 0 | −0.00340487 | −0.00170932 | −0.00266506 | −0.00237264 |
| 56644 | Clec7a | 0.002540312 | 0 | 0 | 0.009893889 | 0.005394719 | 0.00212674 | 0.006100333 |
| 13112 | Cyp3a11 | −0.00254 | 0 | 0 | −0.00360391 | −0.00089639 | −0.00143935 | 0 |
| 67305 | Gpx7 | −0.00253402 | 0 | 0 | −0.00344812 | −0.00245512 | −0.00250998 | −0.00301318 |
| 20343 | Sell | −0.00253272 | 0 | 0 | −0.0054131 | −0.00270377 | −0.0011319 | −0.00146457 |
| 14115 | Fbln2 | −0.00251161 | 0 | 0 | −0.00363387 | −0.00165125 | −0.00180606 | −0.00112786 |
| 110893 | Slc8a3 | 0.002502441 | 0 | 0 | 0.004763667 | 0.002429236 | 0.0022054 | 0.00484479 |
| 14677 | Gnai1 | 0.002500937 | 0 | 0 | 0.001687455 | 0.000665682 | 0.001059813 | 0.000456444 |
| 80859 | Nfkbiz | 0.002497822 | 0 | 0 | 0.002278771 | 0.001437181 | 0.002297149 | 0.001696292 |
| 20519 | Slc22a3 | −0.00249602 | 0 | 0 | −0.00295952 | −0.00126666 | −0.00193975 | −0.00149686 |
| 15792 | Laptm5 | 0.002494537 | 0 | 0 | 0.003904118 | 0.002581151 | 0.002283047 | 0.002900729 |
| 211151 | Churc1 | 0.002491697 | 0 | 0 | 0.001444553 | 0.001284685 | 0.002056744 | 0.001869074 |
| 18294 | Ogg1 | −0.00249118 | 0 | 0 | −0.00212745 | −0.00094969 | −0.00146314 | −0.00043124 |
| 70080 | Igsf23 | 0.002481028 | 0 | 0 | 0 | 0.000682051 | 0.000822577 | 0 |
| 219151 | Scara3 | −0.00247881 | 0 | 0.017831878 | −0.00069464 | −0.00037653 | −0.0035381 | −0.00055857 |
| 18782 | Pla2g2d | 0.002476305 | 0 | 0 | −0.0002905 | 0.002811985 | 0.002227802 | 0.002000476 |
| 12274 | C6 | 0.002474638 | 0 | 0 | −0.00125018 | 0.000596344 | 0.002070018 | 0.00080075 |
| 217845 | Ifi27l2b | 0.002471263 | 0 | 0 | 0.000314012 | 0.00017442 | 0.002027471 | 0 |
| 666168 | Cyp4a31 | −0.00247048 | 0 | 0 | 0 | 0 | −0.00298024 | 0 |
| 20370 | Sez6 | −0.00246567 | 0 | 0 | −0.00016659 | −0.00059249 | 0 | 0 |
| 15201 | Hells | 0.002464914 | 0 | 0 | 0.001408423 | 0.00018491 | 0.001378426 | −0.00030027 |
| 17869 | Myc | 0.002460595 | 0 | 0 | 0.001612336 | 0.000855332 | 0.003261821 | 0.002295618 |
| 12683 | Cidea | 0.002458648 | 0 | 0.008655159 | −0.00107835 | 0.001428853 | 0.00603765 | 0.001919272 |
| 18703 | Pigr | 0.002455627 | 0 | 0 | 0.000977865 | 0.002090991 | 0.002950414 | 0.002707641 |
| 56264 | Cpxm1 | −0.00245506 | 0 | 0 | −0.00132368 | −0.00129061 | −0.00241367 | −0.00127439 |
| 278180 | Vsig4 | 0.002454854 | 0 | 0 | 0.004248443 | 0.004861383 | 0.002641552 | 0.005690704 |
| 15493 | Hsd3b2 | −0.00245482 | 0 | 0 | −0.00227029 | −0.00089574 | −0.00244084 | 0 |
| 171580 | Mical1 | −0.00245334 | 0 | 0 | −0.00315635 | −0.00066066 | −0.00123491 | −0.00053447 |
| 224440 | Setd4 | 0.002451597 | 0 | 0 | 0.001610169 | 0.000524778 | 0.002584907 | 0.001041334 |
| 268354 | Tafa2 | 0.002447565 | 0 | 0 | 0 | 0.000803467 | 0 | 0 |
| 12227 | Btg2 | 0.002444351 | 0 | 0 | 0.002239849 | 0.001488416 | 0.002626589 | 0.001101983 |
| 12364 | Casp12 | 0.002443755 | 0 | 0 | 0.004091285 | 0.002422807 | 0.001690311 | 0.000439441 |
| 12260 | C1qb | 0.002432047 | 0 | 0 | 0.005436088 | 0.003222427 | 0.001992733 | 0.003477382 |
| 25388 | Ifi202b | −0.0024256 | 0 | 0 | 0 | 0 | −0.00165003 | 0 |
| 18573 | Pde1a | 0.002423291 | 0 | 0 | 0.001189107 | 0.000508081 | 0.002569825 | 0.001260207 |
| 66098 | Chchd6 | −0.00242129 | 0 | 0 | −0.00071419 | −0.00056329 | −0.00191263 | −0.00058542 |
| 17868 | Mybpc3 | −0.00242089 | 0 | 0 | −0.00175656 | −0.00130116 | −0.00187023 | 0 |
| 70235 | Poc1a | −0.00242018 | 0 | 0 | −0.00092028 | −0.00094399 | −0.00133462 | −0.00126846 |
| 230971 | Megf6 | −0.00241854 | 0 | 0 | −0.00294514 | −0.00124988 | −0.00117959 | −0.0014442 |
| 233806 | Tmem159 | −0.00241847 | 0 | 0 | −0.00281292 | −0.00140699 | −0.00117948 | −0.00140753 |
| 76850 | Ago4 | −0.00240568 | 0 | 0 | 0.000842265 | 0.000227237 | −0.00060727 | 0.000794601 |
| 19348 | Kif20a | 0.00240553 | 0 | 0 | 0.00203112 | −0.00029754 | 0.001023805 | −0.00134316 |
| 667977 | Gm8909 | −0.00240538 | 0 | 0 | 0 | 0 | −0.00051558 | 0 |
| 110749 | Chaf1b | −0.002392 | 0 | 0 | −0.00220399 | −0.00038074 | −0.00089707 | 0.000438751 |
| 20296 | Ccl2 | 0.002390482 | 0 | 0 | 0.001913674 | 0.002152784 | 0.001079154 | 0.001200446 |
| 70546 | Zdhhc2 | −0.00237387 | 0 | 0 | −0.00489766 | −0.00153759 | −0.0001433 | −0.00096359 |
| 269633 | Wdr85 | 0.002373743 | 0 | 0 | 0.002507918 | 0.000570422 | 0.00048732 | 0.000774187 |
| 16541 | Napsa | −0.00236658 | 0 | 0 | −0.00681196 | −0.00111536 | −0.00165357 | −0.00188755 |
| 56185 | Hao2 | −0.00236429 | 0 | 0 | 0.001019104 | −0.00116655 | −0.00498465 | 0 |
| 230806 | Crybg2 | 0.002357008 | 0 | 0 | 0.003502757 | 0.001881159 | 0.002486742 | 0 |
| 24001 | Tlam2 | 0.002354247 | 0 | 0 | 0.002167773 | 0.001166128 | 0.002574649 | 0.002898874 |
| 26557 | Homer2 | −0.00234769 | 0 | 0 | −0.00096806 | −0.00015355 | −0.00111679 | 0.000882385 |
| 20208 | Saa1 | −0.00234447 | 0 | 0 | 0 | 0 | −0.0021998 | 0 |
| 268780 | Egflam | 0.002335703 | 0 | 0 | 0.00181722 | 0.000800348 | 0.000789341 | −0.00028844 |
| 381801 | Tatdn2 | −0.00233421 | 0 | 0 | −0.0023581 | −0.0010342 | −0.00147871 | −0.00098306 |
| 231655 | Oasl1 | 0.002333007 | 0 | 0 | −0.00186769 | 0.000943652 | 0.003462011 | 0.001431627 |
| 56077 | Dgke | −0.00233114 | 0 | 0 | −0.00162195 | −0.00034797 | −0.00132781 | −0.0007048 |
| 67379 | Dedd2 | −0.0023301 | 0 | 0 | −0.00229344 | −0.00112752 | −0.00117544 | −0.00048984 |
| 68631 | Cryl1 | 0.002327041 | 0 | 0 | −0.00072344 | 0.000479329 | 0.00228799 | −0.00029562 |
| 217593 | Slc25a21 | −0.00232372 | 0 | 0 | −0.00033944 | −0.00073944 | −0.0028124 | 0 |
| 16716 | Ky | 0.002319388 | 0 | 0 | −0.00130679 | −0.00035536 | 0.002955895 | 0 |
| 14060 | F13b | 0.002317891 | 0 | 0 | −0.00137528 | −0.00011359 | 0.001543729 | 0 |
| 13123 | Cyp7b1 | 0.002311907 | 0 | 0 | −0.00197384 | −0.00034764 | 0.000631921 | −0.00141634 |
| 215690 | Nav1 | 0.002303077 | 0 | 0 | 0.002574798 | 0.00041948 | 0.001737718 | 0.000947922 |
| 14733 | Gpc1 | 0.00230189 | 0 | 0 | 0.001617552 | 0.001274185 | 0.003142496 | 0.002857493 |
| 19221 | Ptgfrn | −0.00229824 | 0 | 0 | −0.00278299 | −0.00143842 | −0.00075187 | −0.00126884 |
| 67017 | Fam210b | −0.0022973 | 0 | 0 | −0.00069069 | −0.00030932 | −0.00159893 | −0.00052559 |
| 19387 | Rangap1 | −0.0022972 | 0 | 0 | −0.00293471 | −0.00132934 | −0.00229121 | −0.00183666 |
| 66395 | Ahnak | 0.002296191 | 0 | 0 | 0.005319268 | 0.00255273 | 0.000809818 | 0.001685987 |
| 114601 | Ehbp1l1 | 0.002295778 | 0 | 0 | 0.002270832 | 0.001758569 | 0.001764604 | 0.001683089 |
| 18187 | Nrp2 | 0.002293013 | 0 | 0 | 0.001877921 | 0.001502645 | 0.001796467 | 0.001279852 |
| 50781 | Dkk3 | 0.002292982 | 0 | 0.014389711 | 0.002488228 | 0.001819474 | 0.002398997 | 0.002241013 |
| 12523 | Cd84 | 0.002291699 | 0 | 0 | 0.004432939 | 0.001953145 | 0.001815944 | 0.003386306 |
| 53322 | Nucb2 | −0.00229122 | 0 | 0 | −0.00313646 | −0.00115863 | −0.00206993 | −0.00166542 |
| 71373 | Prr16 | −0.00228988 | 0.010981851 | 0 | 0.000764183 | 0.000680341 | −0.00111573 | −0.00058189 |
| 225884 | Gstp3 | −0.00228376 | 0 | 0 | 0 | −0.00075424 | −0.00269877 | 0 |
| 17855 | Mvk | 0.002279436 | 0 | 0 | 0.001050857 | 0.000639821 | 0.001859055 | 0.000125038 |
| 17105 | Lyz2 | 0.002279255 | 0 | 0 | 0.010291027 | 0.002387671 | 0.00253249 | 0.002210199 |
| 14823 | Grm8 | 0.002277694 | 0 | 0 | 0.001891673 | 0.000775187 | 0.002006757 | 0.001029244 |
| 20500 | Slc13a2 | −0.00227576 | 0 | 0 | −0.00318554 | −0.0014524 | −0.00132645 | −0.00067052 |
| 19736 | Rgs4 | 0.002273551 | 0 | 0 | −0.00092342 | −0.00165492 | 0.002531463 | −0.00028953 |
| 26897 | Acot1 | −0.00227098 | 0 | 0 | −0.00055692 | −0.00060859 | −0.00387523 | −0.00222503 |
| 20532 | Slc3a1 | 0.002262871 | 0 | 0 | 0.002195906 | 0.001203322 | 0.003163755 | 0 |
| 17318 | Mid1 | −0.0022617 | 0 | 0 | −0.00337506 | −0.00058843 | −0.00156637 | −0.00058047 |
| 93674 | Nat8f3 | −0.00225963 | 0 | 0 | 0 | 0 | −0.00203547 | 0 |
| 16371 | Irx1 | 0.002259101 | 0 | 0 | 0.002831954 | 0.001119503 | 0.002072044 | 0.001025395 |
| 13476 | Reep5 | −0.00225748 | 0 | 0 | −0.00153856 | −0.00101738 | −0.0014413 | −0.00084328 |
| 11826 | Aqp1 | 0.00225453 | 0 | 0 | −0.00104157 | 0.000711231 | 0.00211962 | −0.00012903 |
| 71770 | Ap2b1 | −0.00225047 | 0 | 0 | −0.0025329 | −0.00135874 | −0.00249633 | −0.00156387 |
| 65256 | Asb2 | −0.00224285 | 0 | 0 | −0.00418523 | −0.00093704 | −0.00143685 | −0.00183839 |
| 214301 | Crygn | −0.00223814 | 0 | 0 | −0.00483797 | −0.00145719 | −0.0003989 | 0 |
| 69638 | Enho | 0.002237793 | 0 | 0 | 0.004218931 | 0.001006765 | 0.00043955 | 0.00057218 |
| 56642 | Ankrd2 | 0.002235667 | 0 | 0 | 0.002052216 | 0.000368392 | 0.000551785 | 0 |
| 231130 | Tnip2 | −0.00223359 | 0 | 0 | −0.00328742 | −0.00052585 | −0.00112055 | −0.00051671 |
| 217847 | Serpina10 | 0.002232934 | 0 | 0 | 0.002772907 | 0.001159322 | 0.000700633 | 0.000761842 |
| 22351 | Vill | −0.00223123 | 0 | 0 | −0.0011689 | −0.0003125 | −0.00187862 | −0.00050877 |
| 29810 | Bag3 | 0.00222938 | 0 | 0 | 0.003717441 | 0.001390782 | 0.001836622 | 0.001972921 |
| 69861 | 2010003K11Rik | −0.00222626 | 0 | 0 | −0.00342688 | −0.00205126 | −0.0009717 | 0 |
| 15446 | Hpgd | −0.002225 | −0.00656473 | 0 | −0.00223794 | −0.00247167 | −0.00210357 | −0.00239137 |
| 12799 | Cnp | 0.002220182 | 0 | 0 | −0.00028842 | 0.000565443 | 0.002421096 | 0.000453968 |
| 57437 | Golga7 | 0.00221776 | 0 | 0 | 0.001953908 | 0.00104064 | 0.002031827 | 0.001402261 |
| 235587 | Parp3 | 0.002217553 | 0 | 0 | 0.002501311 | 0.001806362 | 0.001842976 | 0.001184354 |
| 20661 | Sort1 | −0.00221438 | 0 | 0 | −0.00391591 | −0.0017951 | −0.00245079 | −0.00290109 |
| 234267 | Gpm6a | −0.00221386 | 0 | 0 | −0.00189094 | −0.00073053 | −0.00182829 | −0.00031683 |
| 321018 | Serpina4-ps1 | −0.00220952 | 0 | 0 | 0 | 0 | −0.00434115 | 0 |
| 67708 | Pcnx4 | 0.002208481 | 0 | 0 | 0.000932349 | 0.00078634 | 0.001732361 | 0.000340124 |
| 106042 | Prickle1 | 0.002207899 | 0 | 0 | 0.000203997 | 7.42E−05 | 0.001170049 | −0.00055207 |
| 212943 | Tent5a | −0.00220774 | 0 | 0 | −0.00231254 | −0.00096241 | −0.00163589 | −0.0010389 |
| 18205 | Ntf3 | 0.002205174 | 0 | 0 | −0.00054576 | −0.00062505 | 0.002464221 | 0 |
| 20249 | Scd1 | 0.002204723 | 0 | −0.00141792 | 0.002612378 | 0.000260585 | 0.002838847 | 0.000951494 |
| 76454 | Fbxo31 | −0.00220275 | 0 | 0 | −0.00311907 | −0.00127166 | −0.00146915 | −0.00080121 |
| 17888 | Myh6 | −0.00220105 | 0 | 0 | −0.00067609 | −0.00088768 | −0.00134047 | 0 |
| 67564 | Tmem35a | −0.00219984 | 0 | 0 | −0.00270575 | −0.00159166 | −0.00133149 | 0 |
| 246278 | Cd207 | 0.002198469 | 0 | 0 | −0.0013321 | 0.000177126 | 0.001240994 | 0 |
| 71761 | Amdhd1 | −0.00219603 | 0 | 0 | −0.00090591 | −0.00119684 | −0.00198186 | 0 |
| 216233 | Sccs2 | −0.00219459 | 0 | 0 | 0.000197321 | −0.00092385 | −0.0007489 | 1.81E−05 |
| 22335 | Vdac3 | 0.002190322 | 0 | 0 | 0.003291427 | 0.001352806 | 0.00118039 | 0.001102444 |
| 243529 | H1f10 | −0.00218929 | 0 | 0 | −0.00267554 | −0.00189297 | −0.0025424 | 0 |
| 12314 | Calm2 | 0.002188922 | 0 | 0 | 0.001582037 | 0.001317504 | 0.001957727 | 0.001857319 |
| 66234 | Msmo1 | 0.002186927 | 0 | 0 | 0.002486031 | 0.001287375 | 0.001743113 | 0.000831969 |
| 14264 | Fmod | −0.00218136 | 0 | 0 | −0.00120678 | −0.0017716 | −0.00190892 | −0.00081994 |
| 104086 | Cyp27a1 | −0.00218 | 0 | 0 | −0.00222701 | −0.00024595 | −0.00161233 | −0.00092715 |
| 67693 | Hypk | 0.002175227 | 0 | 0 | 0.002191553 | 0.001064731 | 0.00143036 | 0.001763237 |
| 71703 | Armcx3 | −0.0021732 | 0 | 0 | −0.00401563 | −0.00145066 | −0.00175015 | −0.00204818 |
| 15168 | Hcn3 | 0.002171959 | 0 | 0 | 0.003015235 | 0.00095456 | 0.001990049 | 0.000881006 |
| 67375 | Qprt | −0.0021663 | 0 | 0 | 0.002206726 | 0.000521668 | −0.00206531 | 0.001138547 |
| 214663 | Slc25629 | −0.00215977 | 0 | 0 | −0.0047517 | −0.00170635 | −0.00189794 | −0.0021744 |
| 79043 | Spsb3 | 0.002154118 | 0 | 0 | 0.002696633 | 0.000871596 | 0.001570982 | 0.001265446 |
| 16870 | Lhx2 | 0.002152978 | 0 | 0 | 0.002462482 | 0.001685196 | 0.001554833 | 0.000463628 |
| 114664 | Hsd17b11 | −0.0021514 | 0 | 0 | −0.00108832 | −0.00074422 | −0.00242017 | −0.00200043 |
| 433022 | Plcxd2 | −0.00214893 | 0 | 0 | 0.000988131 | −0.00108378 | −0.00124907 | 0.000665954 |
| 108030 | Lin7a | −0.00214848 | 0 | 0 | −0.00096733 | −0.00112558 | −0.00158532 | −0.00034232 |
| 240697 | Mcmdc2 | −0.00214573 | 0 | 0 | −0.00245544 | −0.00053443 | −0.00111082 | 0 |
| 72016 | Tedc2 | −0.00214359 | 0 | 0 | −0.00198072 | −0.00099714 | −0.00159492 | −0.00117437 |
| 18030 | Nfil3 | 0.00214343 | 0 | −0.01397618 | 0.002312044 | 0.000723883 | 0.00246185 | 0.001404298 |
| 66990 | Tmem134 | −0.00214278 | 0 | 0 | −0.00338018 | −0.00145754 | −0.00053364 | −0.00070548 |
| 68283 | 9530077C05Rik | 0.00213952 | 0 | 0 | 0.002270477 | 0.000438187 | 0.002092007 | 0.000704323 |
| 114142 | Foxp2 | −0.00213441 | 0 | 0 | −0.00051664 | −0.00025361 | −0.00150349 | 0.00054309 |
| 78514 | Arhgap10 | 0.002134198 | 0 | 0 | 0.002716192 | 0.001201935 | 0.001537589 | 0.002106762 |
| 14869 | Gstp2 | 0.002132812 | 0 | 0 | 0 | 0 | 0.002789733 | 0 |
| 110382 | C8b | −0.00213071 | 0 | 0 | −0.00110517 | −0.0017179 | −0.002999 | 0 |
| 65473 | Ctrb1 | −0.00212953 | 0 | 0 | 0 | −0.0008459 | −0.00159561 | 0 |
| 13122 | Cyp7a1 | 0.002129183 | 0 | 0 | 0.001386482 | 0.000605149 | 0.001453148 | 0 |
| 102545 | Cmtm7 | −0.00212802 | 0 | 0 | −0.00150609 | −0.00022405 | −0.00101709 | 5.07E−05 |
| 56277 | Tmem45a | −0.00212566 | 0 | 0 | −0.00365022 | −0.00070016 | −0.00036926 | −0.00029298 |
| 11835 | Ar | 0.002124124 | 0 | 0 | 0.001374087 | 0.002337743 | 0.002580736 | 0.003914859 |
| 16414 | Itgb2 | 0.002121811 | 0 | 0 | 0.002964245 | 0.00260085 | 0.001859629 | 0.001808767 |
| 56348 | Hsd17b12 | −0.0021196 | 0 | 0 | −0.00227829 | −0.00161985 | −0.00192002 | −0.00162125 |
| 72503 | 2610507B11Rik | −0.00211411 | 0 | 0 | −0.00414096 | −0.00094545 | −0.00206605 | −0.0013909 |
| 105245 | Txndc5 | 0.002112814 | 0 | 0 | 0.002528235 | 0.00132388 | 0.001588494 | 0.00135769 |
| 12986 | Csf3r | 0.002111462 | 0 | 0 | 0.005103875 | 0.002870162 | 0.001886068 | 0.004510996 |
| 140781 | Myh7 | −0.00211138 | 0 | 0 | 0.000995107 | 6.68E−05 | −0.00141926 | −0.00058027 |
| 94109 | Csmd1 | 0.00211011 | 0 | 0 | 0.003713595 | 0.003037431 | 0 | 0.002455176 |
| 83925 | Trps1 | 0.002109986 | 0 | 0 | 0.00309004 | 0.001212342 | 0.001813497 | 0.00065337 |
| 69550 | Bst2 | −0.00210751 | 0 | 0 | −0.00033965 | 0.000684468 | −0.00022297 | 0.002240202 |
| 20750 | Spp1 | −0.00210274 | 0.030613974 | 0 | 0.002436694 | 0.002613367 | −0.00315466 | 0.003473282 |
| 170829 | Tram2 | −0.00210188 | 0 | 0 | −0.00154521 | −0.00072429 | −0.0010498 | −0.00018278 |
| 22190 | Ubc | −0.00210157 | 0 | 0 | −0.004027 | −0.00154095 | −0.00055179 | −0.00105728 |
| 15205 | Hes1 | 0.002101451 | 0 | 0 | 0.002628522 | 0.001436534 | 0.001715911 | 0.000921913 |
| 227157 | Mpp4 | 0.002101355 | 0 | 0 | 0.00183282 | 0.000406919 | 0.002875809 | 0.001390759 |
| 64297 | Gprc5b | −0.00210062 | 0 | 0 | −0.00267077 | −0.00056553 | −0.002422 | −0.00198203 |
| 98376 | Gorab | −0.00209758 | 0 | 0 | −0.00203916 | −0.00050431 | −0.00121848 | −0.00020235 |
| 14675 | Gna14 | 0.002096529 | 0 | 0 | 0.001784666 | 0.00074054 | 0.001749347 | 0.001545104 |
| 21859 | Timp3 | 0.0020925 | 0.000744224 | 0 | 0.000730694 | 0.001820531 | 0.00215867 | 0.001509321 |
| 21847 | Klf10 | 0.002091331 | 0 | 0 | −0.00031959 | 0.000219124 | 0.001552552 | −0.00036748 |
| 21391 | Tbxas1 | −0.00209087 | 0 | 0 | −0.00163748 | 0.000281927 | −0.00090741 | 0.000448626 |
| 246102 | Rttn | −0.00208857 | 0 | 0 | −6.06E−05 | 0.000295844 | −0.00130853 | 0.000291859 |
| 72043 | Sulf2 | −0.00208747 | 0 | 0 | −0.00129026 | −0.00068631 | −0.00127953 | −0.00091297 |
| 53612 | Vti1b | −0.00208697 | 0 | 0 | −0.00229815 | −0.00095996 | −0.00202534 | −0.001545 |
| 235674 | Acaa1b | −0.00208165 | 0 | 0 | −0.00173127 | −0.00139212 | −0.00181673 | 0 |
| 19899 | Rpl18 | 0.002080874 | 0 | 0 | 0.001784798 | 0.000549156 | 0.000409852 | −0.00022877 |
| 16483 | Kap | −0.00207909 | 0 | 0 | 0 | 0.000217195 | 0 | 0 |
| 55807 | Scamp5 | −0.00207732 | 0 | 0 | −0.00362915 | −0.00128703 | −0.00105751 | −0.0007654 |
| 276950 | Slfn8 | 0.002077307 | 0.008918795 | 0 | 0.00456162 | 0.002320987 | 0.001710527 | 0.002685482 |
| 14686 | Gnat2 | −0.00207629 | 0 | 0 | −0.00145744 | −0.00112113 | −0.00208103 | 0 |
| 54371 | Chst2 | −0.00207505 | 0 | 0 | −0.00284655 | −0.00154943 | −0.00176354 | −0.00253831 |
| 20525 | Slc2a1 | −0.00207402 | 0 | 0 | −0.00012556 | −0.00027941 | −0.00119319 | 0.000868105 |
| 12827 | Col4a2 | −0.00207274 | 0 | 0 | −0.00299995 | −0.001813 | −0.00249056 | −0.00305578 |
| 239447 | Colec10 | −0.00207192 | 0 | 0 | −0.00438061 | −0.00113006 | −0.00127266 | −0.00136414 |
| 66686 | Dcbld1 | −0.00206072 | 0 | 0 | −0.00265938 | −0.00152703 | −0.00099379 | −0.00161338 |
| 66970 | Ssbp2 | −0.00206059 | 0 | 0 | 0.001043443 | 0.000614797 | −0.00081142 | 0.001546881 |
| 320981 | Enpp6 | 0.002058019 | 0 | 0 | −0.00074254 | −0.00125938 | 0.002016433 | −0.00163894 |
| 20104 | Rps6 | 0.002056527 | 0 | 0 | 0.001928651 | 0.000675951 | 0.00148047 | 0.00079122 |
| 76574 | Mfsd2a | −0.0020561 | 0 | 0 | −0.00490416 | −0.00240729 | 0.000535572 | −0.00202145 |
| 58193 | Rpl24 | 0.002055216 | 0 | 0 | 0.000821225 | 0.001065243 | 0.002805375 | 0.002283295 |
| 21956 | Tnnt2 | −0.00205416 | 0 | 0 | 0.000303053 | 0.001793227 | −0.00180027 | 0 |
| 15068 | Il18bp | 0.002053727 | 0 | 0 | 0.000784157 | 0.000814909 | 0.000766977 | 0.000569268 |
| 214191 | Ttc24 | −0.00205282 | 0 | 0 | 0 | 0 | −0.00127826 | 0 |
| 105428 | Fam149b | −0.0020493 | 0 | 0 | −0.00357826 | −0.00105761 | −0.0011137 | −0.00109464 |
| 240776 | Kcnt2 | 0.002046248 | 0 | 0 | 0.003721709 | 0.001823508 | 0.001453774 | 0.001755395 |
| 23882 | Gadd45g | −0.00204471 | 0 | 0 | −0.00148094 | −0.00110738 | −0.00124513 | −0.0008128 |
| 14120 | Fbp2 | 0.002040968 | 0 | 0 | 0.003180454 | 0.002937961 | 0.00151505 | 0 |
| 17288 | Mep1b | 0.002038127 | 0 | 0 | 0.000968382 | 0.000504644 | 0.002074086 | 0 |
| 245038 | Dclk3 | 0.002036879 | 0 | 0 | 0.000448051 | 0.000353832 | 0.000411785 | −0.00042005 |
| 11980 | Atp8a1 | 0.002034343 | 0 | 0 | 0.001837407 | 0.001704345 | 0.002124312 | 0.002056731 |
| 55320 | Dbn1 | −0.00203401 | 0 | 0 | −0.00219487 | −0.00124285 | −0.00091868 | −0.00101864 |
| 12349 | Car2 | −0.00203254 | 0 | 0 | −0.00059777 | −0.00042637 | −0.00189996 | −0.00057324 |
| 14219 | Con2 | 0.002029754 | 0 | 0 | −4.73E−05 | 8.56E−05 | 0.00074254 | −0.00026668 |
| 226654 | Tstd1 | 0.002027969 | 0 | 0 | 0.002274865 | 0.000969037 | 0.000448421 | 0 |
| 109791 | Clps | −0.00202581 | 0 | 0 | 0 | −0.00023678 | −0.00117273 | 0 |
| 70082 | Lysmd2 | −0.00202369 | 0 | 0 | −0.00244667 | −0.00068916 | −0.00271455 | −0.00333326 |
| 12111 | Bgn | 0.002023518 | 0 | 0 | 0.002105186 | 0.001376976 | 0.001788971 | 0.002293448 |
| 22283 | Ush2a | 0.002022275 | 0 | 0 | 0.002321039 | 0.000643045 | 0.002136801 | 0.000807716 |
| 235469 | Zfp280d | 0.002020839 | 0 | 0 | 0.000746493 | 0.000779262 | 0.001854893 | 0.001003628 |
| 108098 | Med21 | −0.00201953 | 0 | 0 | −0.00287679 | −0.00126255 | −0.00191102 | −0.00145262 |
| 16518 | Kcnj2 | −0.00201919 | 0 | 0 | 0.000636046 | −1.32E−05 | −0.00149901 | 0.000647614 |
| 79196 | Osbpl5 | −0.00201623 | 0 | 0 | −0.00112354 | −0.00129719 | −0.00125233 | −0.00130885 |
| 245269 | Nim1k | −0.00201605 | 0 | 0 | −0.00203987 | −0.0015453 | −0.0017052 | −0.00206206 |
| 17960 | Nat1 | 0.002014599 | 0 | 0 | 0 | 0 | 0.002148528 | 0 |
| 20715 | Serpina3g | −0.00201211 | 0 | 0 | −0.00149231 | −0.00051639 | −0.00135567 | 0 |
| 13835 | Epha1 | −0.00201173 | 0 | 0 | −0.00202984 | −0.00067997 | −0.00154554 | 0.0013058 |
| 59020 | Pdzk1 | 0.002011172 | 0 | 0 | 0.001821458 | 0.001530972 | 0.001436123 | 0.002468431 |
| 225215 | Rsl24d1 | 0.002010424 | 0 | 0 | 0.002211396 | 0.000876363 | 0.001924991 | 0.001440796 |
| 236900 | Pdk3 | 0.002007749 | 0 | 0 | 0.00376403 | 0.001271663 | 0.000893715 | 6.83E−05 |
| 75530 | Lyrm7 | 0.002007429 | 0 | 0 | 0.001534389 | 9.03E−05 | 0.000717331 | −0.00012963 |
| 14533 | Bloc1s1 | 0.002007191 | 0 | 0 | 0.0008853 | 0.000339119 | 0.002244768 | 0.002035636 |
| 20607 | Sstr3 | −0.00200497 | 0 | 0 | 0 | −0.00129942 | 0 | 0 |
| 12554 | Cdh13 | 0.002003823 | 0 | 0 | −0.00043808 | 0.000907111 | 0.001957635 | −0.00055146 |
| 80794 | Cblc | 0.002000917 | 0 | 0 | −0.00078187 | 0.000269507 | 0.001238575 | −0.00024025 |
| 19261 | Sirpa | 0.00200089 | 0 | 0 | 0.001250931 | 0.001742322 | 0.001047554 | 0.00025347 |
| 72027 | Slc39a4 | −0.00200082 | 0 | 0 | 0.000524292 | −9.08E−05 | −0.00284822 | −0.00085573 |
| 69938 | Scrn1 | 0.002000469 | 0 | 0 | −0.0002516 | 0.000554427 | 0.001647036 | −0.00111494 |
| 236293 | Slc22a29 | 0.001996394 | 0 | 0 | 0 | 0 | 0.000975669 | 0 |
| 12259 | C1qa | 0.001993002 | 0.008982531 | 0 | 0.003328912 | 0.003341616 | 0.00139501 | 0.003701158 |
| 237256 | Zc3h12d | 0.001988265 | 0 | 0 | 0.002489863 | 0.001393676 | 0.002377683 | 0.0024265 |
| 14313 | Fst | 0.001987249 | 0 | 0 | 0.005014487 | 0.001469981 | 0.000975768 | 0.002066966 |
| 54385 | Cyp4f14 | −0.00198607 | 0 | 0 | −0.00173588 | −0.0009085 | −0.00158447 | 0 |
| 218121 | Mbcat1 | −0.00198147 | 0 | 0 | −0.0022725 | −0.00091606 | −0.00111397 | −8.03E−05 |
| 16952 | Anxa1 | 0.001980441 | 0 | 0 | 0.00489522 | 0.001966324 | 0.002208635 | 0.002917242 |
| 66689 | Klhl28 | −0.00197902 | 0 | 0 | −0.00318384 | −0.00120666 | −0.00090272 | −0.00196601 |
| 245404 | Dcaf12l1 | −0.00197823 | 0 | 0 | −2.46E−05 | −1.07E−05 | −0.00169519 | −0.0005114 |
| 232334 | Vgll4 | −0.00197815 | 0 | 0 | −0.00300061 | −0.00090956 | −0.00166765 | −0.00145547 |
| 225289 | AW554918 | −0.00197785 | 0 | 0 | −0.00152219 | 7.27E−05 | −0.0005391 | 7.10E−06 |
| 16987 | Lss | 0.001977089 | 0 | 0 | 0.002128989 | 0.001630472 | 0.002192775 | 0.001121206 |
| 54403 | Slc4a4 | 0.00197565 | 0 | 0 | 0.001071269 | 0.000938838 | 0.001891543 | 0.001815279 |
| 24057 | Sh3yl1 | 0.001971568 | 0 | 0 | 0.001202118 | 0.000846603 | 0.002119537 | 0.000280904 |
| 269799 | Clec4a1 | −0.00197136 | 0 | 0 | −0.00167456 | 0.000530316 | −0.00237425 | 5.81E−06 |
| 14368 | Fzd6 | −0.00197009 | 0 | 0 | −0.00100874 | −0.00071201 | −0.00111404 | 0.000336159 |
| 15803 | Lbp | 0.001967458 | 0 | 0 | −0.0016267 | 0.000301847 | 0.002344837 | 0.000196914 |
| 20342 | Selenbp2 | −0.00196458 | 0 | 0 | 0 | 0 | −0.00262491 | 0 |
| 17540 | Irag1 | 0.00196404 | 0 | 0 | 0.003118295 | 0.000759553 | 0.001376378 | 0 |
| 28015 | Polr2m | 0.001962367 | 0 | 0 | 0.002413491 | 0.001056321 | 0.001463844 | 0.001535898 |
| 212032 | Hk3 | −0.00196187 | 0 | 0 | −0.00349837 | −0.00120779 | −0.00044681 | 0.000169393 |
| 18162 | Npr3 | −0.00195995 | 0 | 0 | 0.000834658 | 0.000815005 | −0.00117201 | 0.000792633 |
| 72993 | Appl1 | −0.00195933 | 0 | 0 | −0.00165299 | −0.00064105 | −0.00118563 | −0.00088204 |
| 20266 | Scn1b | 0.001959131 | 0 | 0 | 0.00315691 | 0.001935161 | 0.00161679 | 0.000967372 |
| 78177 | Ninl | −0.00195871 | 0 | 0 | 0.001057886 | 0.001090395 | −0.001195 | 0.002079518 |
| 106757 | Catsperd | −0.00195868 | 0 | 0 | 0 | −0.00044083 | −0.00037066 | 0 |
| 14062 | F2r | 0.001958257 | 0 | 0 | 0.000468205 | 0.000212148 | 0.000318084 | −0.00056416 |
| 233744 | Spon1 | 0.001958027 | 0 | 0 | 0.002256729 | 0.000895124 | 0.000848044 | −0.00078161 |
| 12830 | Col4a5 | −0.00195638 | 0 | 0 | −0.00226521 | −0.00300457 | −0.00236445 | −0.00335375 |
| 19041 | Ppl | −0.00195629 | 0 | 0 | −0.00277918 | 0.000147964 | −0.00100099 | −0.00032293 |
| 52033 | Pbk | −0.0019546 | 0 | 0 | 0.000760291 | 0.000133498 | −0.00123763 | 0 |
| 108150 | Galnt7 | −0.00195402 | 0 | 0 | 6.53E−05 | −0.00033798 | −0.00156834 | 0.000817528 |
| 103967 | Dnm3 | 0.001953482 | 0 | 0 | 0.003271309 | 0.001334112 | 0.001947555 | 0.001978528 |
| 70012 | Cep85 | 0.001953389 | 0 | 0 | 0.003864856 | 0.001369505 | 0.001089053 | 0.001232622 |
| 246133 | Kcne2 | −0.0019507 | 0 | 0 | −0.00407892 | −0.00185475 | −0.00076761 | 0 |
| 12305 | Ddr1 | −0.00194939 | 0 | 0 | −0.00302485 | −0.00095842 | −0.00158172 | −0.00109214 |
| 226781 | Slc30a10 | −0.00194844 | 0 | 0 | −0.00173711 | 0.000335031 | −0.00079483 | 0.000629815 |
| 14231 | Fkbp7 | 0.001948146 | 0 | 0 | 0.000636279 | −0.00039603 | 0.001779433 | −0.00019795 |
| 12160 | Bmp5 | −0.00194775 | 0 | 0 | −0.00337742 | −0.00130508 | −0.00121936 | −0.0010117 |
| 29818 | Hspb7 | −0.00194753 | 0 | 0 | −0.00409936 | −0.00022538 | −0.00179889 | 0.000182119 |
| 20400 | Sh2d1a | 0.00194752 | 0 | 0 | 0 | 0.000918023 | 0.001959993 | 0 |
| 68603 | Pmvk | 0.001939364 | 0 | 0 | 0.000207631 | 0.000801153 | 0.002059309 | 0.00073359 |
| 16880 | Lifr | 0.001938565 | 0 | 0 | 0.001121551 | 0.000741783 | 0.001633822 | 0.000186082 |
| 17087 | Ly96 | −0.00193663 | 0 | 0 | −0.00377041 | −0.00164707 | −0.00120979 | −0.00207083 |
| 16431 | Itm2a | 0.001935996 | 0 | 0 | −0.00440817 | −0.00203336 | 0.000742863 | −0.00381479 |
| 22630 | Ywhaq | −0.00193523 | 0 | 0 | −0.00224045 | −0.00048645 | −0.00074975 | −0.00069502 |
| 14580 | Gfap | 0.00029146 | 0.136147224 | 0 | 0.004525072 | 0.001984508 | 6.29E−05 | 0 |
| 574402 | Gpr17 | 0.000863108 | −0.07725053 | 0 | −0.0039972 | −0.00204374 | 0.00065059 | −0.00270527 |
| 12494 | Cd38 | 0.000561742 | −0.05497855 | 0 | −0.00385334 | −0.00196098 | 0.001002191 | −0.00224885 |
| 93710 | Pcdhga2 | 0 | 0.053488797 | 0 | 0.004775665 | 0.002262127 | 0 | 0 |
| 20787 | Srebf1 | −0.00032285 | 0.042569497 | 0 | 0.001089407 | 0.001275319 | 0.000278148 | 0.001251961 |
| 105243 | Slc9a3 | 0 | 0.038303917 | 0 | −0.00130548 | −7.15E−05 | 0 | 0 |
| 64706 | Scube1 | 0.001219898 | −0.03806417 | 0 | −0.00292279 | −0.00022883 | 0 | 0 |
| 93880 | Pcdhb9 | 0 | 0.037840188 | 0 | 0.006542407 | 0.002278745 | 0 | 0 |
| 11688 | Alox8 | −0.00063815 | 0.035902161 | 0 | 0.004113961 | 0.00192097 | −0.00077595 | 0 |
| 19277 | Ptpro | −0.00144701 | −0.03564834 | 0 | −0.00297091 | −0.00117336 | −0.0007716 | −0.00082502 |
| 381823 | Apold1 | 0.000624024 | 0.033904336 | 0 | 0.000553472 | 0.001030197 | 0.00031766 | −0.0004124 |
| 19143 | St14 | 0.000286882 | 0.032161433 | 0 | 0.00177027 | 0.001863371 | 0.000608448 | 0.001438615 |
| 29870 | Gtse1 | 0.000921435 | −0.03202956 | 0 | −0.00048774 | −5.94E−05 | 0.000625927 | 0.000202526 |
| 68895 | Rasl11a | −0.00049076 | −0.03123047 | 0 | 0.000679973 | −0.00059548 | −0.00052988 | 0.000486523 |
| 12877 | Cpeb1 | −0.00161194 | 0.030756859 | 0 | 0.003647804 | 0.003590812 | −0.00023384 | 0.003715121 |
| 13106 | Cyp2e1 | −1.88E−06 | 0.030238546 | 0 | −0.00133443 | −0.00127495 | 0.000140562 | −0.00176702 |
| 12814 | Col11a1 | 0 | −0.0286068 | 0 | −0.00297575 | −0.0032647 | 0 | −0.00261637 |
| 52712 | Zkscan6 | 0.000404589 | −0.02859824 | 0 | −0.00222889 | −0.00115806 | 0.00014014 | −0.00072055 |
| 11475 | Acta2 | 0.001454655 | −0.02701727 | 0 | 0.001614238 | −0.00027191 | 0.000434241 | 0.000177339 |
| 21823 | Th | −2.45E−05 | 0.026482569 | 0 | 0 | 0.000450839 | −0.00012828 | 0 |
| 16149 | Cd74 | 0.001098135 | 0.025831932 | 0 | 0.003218307 | 0.003994134 | −0.00055505 | 0.002857257 |
| 78781 | Zc3hav1 | −0.00021388 | 0.025643585 | 0 | 0.002941699 | 0.00151632 | −0.00011531 | 0.001968653 |
| 16012 | Igfbp6 | −0.00033373 | 0.025607115 | 0 | −0.00112346 | −0.00086555 | 7.26E−06 | −0.00120502 |
| 100039795 | Ildr2 | 0.001319734 | 0.025481237 | 0 | 0.002552364 | 0.001194012 | 0.001319285 | 0.002807932 |
| 12807 | Hps3 | −0.00057942 | −0.02520464 | 0 | −0.00417485 | −0.00131288 | −0.00016707 | −0.00171186 |
| 93873 | Pcdhb2 | 0 | 0.024075744 | 0 | 0 | 0.001086412 | 0 | 0 |
| 20322 | Sord | 0.001658494 | 0.02243627 | 0 | 0.006356773 | 0.002234689 | 0.000964629 | 0.002795156 |
| 59054 | Mrps30 | −0.00062883 | 0.022263115 | 0 | 0.00196191 | 0.001457434 | 5.71E−07 | 0.001477956 |
| 238271 | Kcnh5 | 0.000101268 | −0.02193409 | 0 | −0.00369076 | −0.00168529 | 4.80E−05 | 0 |
| 19039 | Lgals3bp | 0.001867979 | 0.021282187 | 0 | 0.004907459 | 0.002825204 | 0.00087463 | 0.002716131 |
| 246104 | Rhbdl3 | −0.00063046 | −0.02118228 | 0 | −0.00440363 | −0.00191522 | −0.0008079 | −0.00171422 |
| 65116 | Prrg2 | −0.00020485 | −0.02115571 | 0 | −0.00184753 | −0.00060849 | −0.00036873 | −0.00079456 |
| 102614 | Rpp25 | −0.00020716 | 0.02112299 | 0 | 5.77E−05 | 0.00079668 | −0.00080475 | 0 |
| 69926 | Dnah17 | 0.00085391 | −0.02095685 | 0 | −0.00123192 | −9.43E−05 | −4.51E−05 | 0 |
| 17357 | Marcksl1 | −0.00124135 | −0.02082366 | 0 | −0.00330209 | −0.001654 | −0.00088525 | −0.00209279 |
| 319480 | Itga11 | −0.00063691 | −0.02065499 | 0 | −0.0026867 | −0.00140145 | −0.00086769 | −0.00189654 |
| 14412 | Slc6a13 | 0.000191705 | −0.01952786 | 0 | 0.000136596 | −0.00122853 | 0.000296573 | −0.00241534 |
| 73914 | Irak3 | 0.000168079 | 0.019475951 | 0 | 0.002653787 | 0.002470889 | 0.001743572 | 0.004480604 |
| 24088 | Tlr2 | −0.00096128 | 0.019214019 | 0 | 0.001342443 | 0.001227175 | 0.00013077 | 0.002721925 |
| 54132 | Pdlim1 | 0.000193628 | 0.01913509 | 0 | 0.001310893 | 0.000909938 | 0.001263774 | 0.001461085 |
| 208890 | Slc26a7 | 0 | −0.01911677 | 0 | −0.00103665 | −0.00076156 | 0 | 0 |
| 11865 | Arntl | 0.001578844 | −0.0190395 | 0 | 0.004301912 | −0.00012066 | 0.001768918 | 0.000713321 |
| 237928 | Phospho1 | 0.001281405 | −0.01895303 | 0 | −0.00042395 | −6.21E−05 | 0.001636179 | 0.000834213 |
| 18542 | Pcolce | 0.00029065 | −0.01833317 | 0 | 1.93E−05 | −0.00118115 | −0.00043225 | −0.00119194 |
| 114249 | Npnt | 0.00032845 | −0.01793503 | 0 | −0.00238604 | −0.00196716 | 0.000681139 | −0.00201812 |
| 55990 | Fmo2 | −9.92E−05 | 0.017711805 | 0 | 0.005329526 | 0.002953366 | −0.00062198 | 0.004243515 |
| 11898 | Ass1 | −0.00120764 | 0.017287158 | 0 | 0.001063451 | 0.001111799 | 0.00123666 | 0.001237376 |
| 108115 | Slcc4a1 | 0.000176229 | −0.017207 | 0 | −0.00268975 | 0.00023985 | 0.000119779 | −0.00085181 |
| 217779 | Lysmd1 | −0.00017118 | −0.01678511 | 0 | −0.00241967 | −0.00162999 | −0.00036522 | −0.00132349 |
| 329628 | Fat4 | 0.000496205 | −0.01656247 | 0 | −0.00289315 | −0.00178234 | −0.0003605 | −0.00200242 |
| 268527 | Greb1 | 0.00027023 | 0.016455673 | 0 | 0.001708406 | 0.000218173 | 0.000149746 | 0 |
| 11438 | Chrna4 | 0.000300571 | 0.016304117 | 0 | 0.001847767 | 0.000708239 | −0.00127082 | −0.00129245 |
| 18682 | Phkg1 | 0.000884346 | −0.01615317 | 0 | −0.0040305 | −0.00204925 | 0.00015587 | −0.00165204 |
| 13386 | Dlk1 | 0 | 0.01593126 | 0 | 0.00255095 | 0.001181054 | 0 | 0 |
| 22063 | Trpc1 | 0.000724838 | 0.015577762 | 0 | 0.004251632 | 0.002746006 | 0.001673367 | 0.004522764 |
| 93840 | Vangl2 | −0.00074679 | −0.01548687 | 0 | −0.0032009 | −0.00256292 | −0.0008682 | −0.00232507 |
| 66898 | Baiap2l1 | −0.00122739 | 0.015054953 | 0 | 0.001953787 | −5.03E−05 | −0.00074601 | 0.000765749 |
| 80892 | Zfhx4 | 0.000941723 | 0.014915873 | 0 | 0.001687507 | 0.001549109 | 7.47E−05 | 0.000990422 |
| 269437 | Plch1 | 0.000320679 | −0.01476867 | 0 | −0.00204233 | −0.00092724 | 0.00037338 | 0 |
| 108800 | Ston2 | 0.000254245 | −0.01459489 | 0 | −0.00099318 | −0.00087634 | −0.00032413 | −0.00163322 |
| 19739 | Rgs9 | 0.00074527 | −0.01368355 | 0 | −0.00149034 | −0.00109457 | −0.00041891 | −0.00242367 |
| 627626 | Ptchd4 | 0 | −0.0131875 | 0 | −0.00065876 | −0.00033291 | 0 | 0 |
| 233836 | Slc5a11 | 0 | −0.01270184 | 0 | 0 | 0.000188766 | 0 | 0 |
| 66089 | Rmnd5b | −0.00048536 | −0.01260404 | 0 | −0.00135836 | −0.00101513 | −5.12E−05 | −0.00103639 |
| 17349 | Mlf1 | 0.000108421 | −0.01254552 | 0 | −0.00284161 | −0.00241492 | −9.89E−05 | −0.00250702 |
| 382252 | Bciaf3 | −0.00024026 | −0.0124916 | 0 | 0.00010726 | −0.00048927 | −0.00021347 | 0.000239484 |
| 78267 | Klhdc8b | −8.39E−05 | −0.01242706 | 0 | −0.00266759 | −0.0014097 | −0.00045125 | −0.001402 |
| 20776 | Tmie | 0.001209253 | 0.012423416 | 0 | 0.004178043 | 0.001488626 | 6.45E−05 | 0.001093916 |
| 105440 | Kctd9 | −0.00111239 | −0.01235751 | 0 | −0.00179365 | −0.00090989 | −0.00080149 | −0.00045994 |
| 231999 | Plekha8 | −0.00060874 | −0.01219447 | 0 | −0.00150189 | −0.0009308 | −0.00033808 | −0.00063051 |
| 52463 | Tet1 | −0.0001554 | −0.01150607 | 0 | −0.00394652 | −0.00299359 | −0.00045277 | −0.00351906 |
| 12010 | B2m | 0.000888301 | 0.011479903 | 0 | 0.003685079 | 0.00279824 | 0.000915615 | 0.002553415 |
| 50917 | Galns | 0.000442981 | 0.011120997 | 0 | 0.000993685 | 0.000884471 | 0.000618198 | 0.000541842 |
| 18217 | Ntsr2 | 0 | 0.011044889 | 0 | 0.002761641 | 0.001365878 | 0 | 0 |
| 54357 | Epb41l4b | −0.00022044 | 0.010697682 | 0 | 0.002651065 | 0.000830316 | −0.00045375 | 0.000931058 |
| 239337 | Adamts12 | −3.08E−05 | −0.01069562 | 0 | −0.00315453 | −0.00281718 | 0 | −0.00271644 |
| 110959 | Nudt19 | 0.000192367 | 0.010446581 | 0 | 0.00178918 | 0.000915095 | −0.00018481 | 0.000245839 |
| 237213 | Glra2 | 0 | −0.01037579 | 0 | 0 | 0 | 0 | 0 |
| 24117 | Wif1 | 0.00022848 | −0.01009909 | 0 | 0.000318574 | −9.91E−05 | 0.001028466 | 0.00168165 |
| 17932 | Myt1 | −5.87E−05 | −0.01007772 | 0 | −0.00225551 | −0.00113852 | −0.0004512 | 0 |
| 15114 | Hap1 | −0.00067486 | 0.010052309 | 0 | 0.001594969 | 0.001387995 | −0.00115172 | 0.000364937 |
| 216578 | Papolg | 0.000855256 | −0.00997164 | 0 | −0.00108592 | −0.00090117 | 0.00040343 | −0.00074131 |
| 72852 | Mblac2 | −7.49E−05 | 0.009750583 | 0 | 0.00201999 | 0.001202449 | −0.00058391 | 0.000276056 |
| 215387 | Ncaph | −0.00010722 | −0.00968111 | 0 | −0.00245887 | −0.00225901 | −0.00072826 | −0.00303037 |
| 14706 | Gng4 | −0.00052213 | 0.009537112 | 0 | 0.000967147 | 0.001108056 | −0.00049843 | 0.00199859 |
| 22177 | Tyrobp | 0.001581886 | 0.009487064 | 0 | 0.002864651 | 0.002085102 | 0.001496191 | 0.002390932 |
| 19224 | Ptgs1 | −0.00038814 | 0.009069487 | 0 | 0.000917334 | 0.000996293 | −0.0003051 | 0.000264832 |
| 69786 | Tprkb | 0.000563939 | −0.00899222 | 0 | 0.000465807 | −5.06E−05 | 0.000830177 | 0.00053317 |
| 433323 | Sgpp2 | 0.000231253 | 0.008785346 | 0 | 0.003407907 | 0.002386154 | 0.000219227 | 0 |
| 210293 | Dock10 | −0.0007267 | −0.00868517 | 0 | −0.00351504 | −0.00176705 | −0.00054517 | −0.00228703 |
| 70918 | Nsun7 | 7.29E−05 | −0.00855139 | 0 | −0.0017868 | −0.00076464 | −0.00012087 | 0 |
| 71897 | Lypd6b | 0.000167879 | 0.00842774 | 0 | 0.00116707 | 0.000622671 | −0.00011934 | 0 |
| 26949 | Vat1 | 0.000103008 | 0.008251063 | 0 | 0.003720341 | 0.002011872 | 0.000988213 | 0.003364403 |
| 108797 | Mex3b | 6.32E−05 | −0.00815701 | 0 | −0.00220364 | −0.00179214 | −0.00028997 | 0 |
| 20055 | Rps16 | 0.000505154 | −0.00787078 | 0 | −0.00196142 | −0.00114731 | −0.00133853 | −0.00340858 |
| 52626 | Cdkn2aipnl | −0.00017796 | 0.007856589 | 0 | 0.000767409 | 0.001303939 | −0.00029509 | 0.000888861 |
| 13143 | Dapk2 | 0.000563142 | −0.00781433 | 0 | −0.00141473 | −0.00044319 | 0.000956784 | −0.00128578 |
| 75646 | Rai14 | 0.000530967 | 0.007792801 | 0 | 0.004912745 | 0.002497026 | 0.00065826 | 0.003066094 |
| 109229 | Fam118b | −0.00120634 | −0.00757792 | 0 | −0.00241555 | −0.00150016 | −0.00122937 | −0.00178027 |
| 12266 | C3 | 0.000125298 | 0.007273468 | 0 | 0.004093039 | 0.002482072 | 0.000367706 | 0.004391932 |
| 207596 | Thsd4 | 3.24E−05 | −0.00720199 | 0 | −0.00027149 | −0.00048182 | 0.000113325 | 0.000194611 |
| 53324 | Nptx2 | −0.00115898 | −0.0071409 | 0 | −0.00345999 | −0.00167156 | −0.00107502 | 0 |
| 12426 | Cckbr | −6.04E−05 | 0.007116242 | 0 | 0.001168018 | 0.00075607 | −0.00015377 | 0 |
| 12955 | Cryab | 0.000969977 | 0.007034627 | 0 | −0.00032558 | 0.001073568 | 0.000651352 | −0.00047299 |
| 380614 | Intu | −0.0006365 | 0.006934988 | 0 | 0.000244488 | 0.000592415 | −0.00050284 | 0.000474716 |
| 226098 | Hectd2 | 0.000820326 | −0.00659255 | 0 | −0.00133404 | −0.00029721 | 0.000153332 | −0.00066247 |
| 23999 | Twf2 | −3.75E−05 | −0.00658275 | 0 | −0.00338292 | −0.0021327 | 0.000121403 | −0.00250981 |
| 12380 | Cast | −0.00032293 | 0.006558009 | 0 | 0.001788504 | 0.000939754 | −3.29E−05 | 0.001036033 |
| 93707 | Pcdhgc4 | 1.58E−05 | −0.00654502 | 0 | −0.00190274 | −0.0011105 | 9.32E−05 | −0.00146232 |
| 72018 | Fundc1 | 0.000415147 | 0.006433831 | 0 | 0.000931804 | 0.00051734 | 0.000546887 | 0.000279449 |
| 66606 | Lrrc57 | 4.22E−05 | −0.00628773 | 0 | −0.00045117 | −0.00037218 | 0.00055318 | 0.000526039 |
| 26934 | Racgap1 | −0.00078318 | −0.00627815 | 0 | −0.00307824 | −0.00227501 | −0.001112 | −0.00287903 |
| 83768 | Dpp7 | −0.00130284 | 0.006248397 | 0 | −0.00080184 | 0.000113876 | −0.00052389 | −0.00024915 |
| 22325 | Vav2 | 0.0005902 | −0.0061927 | 0 | −0.00239151 | −0.00126105 | 0.000546525 | −0.00196356 |
| 73162 | Otud3 | 0.000890055 | −0.00601034 | 0 | −0.00072163 | −0.00145441 | 0.000759574 | −0.00096792 |
| 93887 | Pcdhb16 | 0.00034839 | 0.005917161 | 0 | 0.003439801 | 0.001854793 | 0.000574973 | 0.002689406 |
| 69297 | Lrrc46 | 0.000282641 | −0.00587674 | 0 | −0.00150351 | −0.00104237 | 0.00069295 | −0.00089189 |
| 22290 | Uty | 0.001593853 | 0.005674252 | 0 | 0.000888027 | 0.001337217 | 0.002187599 | 0.002760817 |
| 269941 | Chsy1 | 0.000575305 | 0.005668303 | 0 | 0.000974712 | 0.000869759 | 0.000829827 | −0.00037989 |
| 12839 | Col9a1 | −0.00092483 | −0.00560475 | −0.01688326 | −0.00504553 | −0.00312392 | −0.00032988 | −0.00312432 |
| 223827 | Gxylt1 | −0.00138818 | 0.005569795 | 0 | 0.000674418 | 0.000731301 | −0.00025484 | 0.000768828 |
| 75600 | Calml4 | −0.00056242 | −0.0054714 | 0 | −0.00145319 | −0.00083163 | −0.0012565 | −0.00037732 |
| 59031 | Chst12 | 0.000984502 | −0.00543085 | 0 | 1.57E−05 | −2.08E−05 | 0.00039718 | −0.00047334 |
| 93891 | Pcdhb20 | −0.00091518 | 0.005386038 | 0 | 0.003678292 | 0.001245247 | −0.00043601 | 0.000836752 |
| 71918 | Zcchc24 | −0.0002559 | 0.005377354 | 0 | 3.88E−05 | 0.00090918 | −0.00029175 | 0.000391953 |
| 58250 | Chst11 | −0.00062641 | −0.00531562 | 0 | −0.00141412 | −0.00095333 | 0 | −0.00110701 |
| 68770 | Phtf2 | −0.00092253 | 0.005087957 | 0 | 0.001405182 | 0.0008484 | −0.00121692 | 0.0007226 |
| 67405 | Nts | −0.00012354 | −0.00505743 | 0 | −0.00078545 | −0.00111955 | 1.17E−05 | 0 |
| 381853 | Gipr | 0 | 0.004975082 | 0 | 0 | 0.000960618 | 0 | 0 |
| 231134 | Dok7 | 3.45E−05 | 0.004941838 | 0 | 0.001359545 | 0.00076633 | −2.05E−05 | 0.000257536 |
| 13170 | Dbp | 0.001288114 | 0.004871996 | 0.00562895 | 0.000731154 | 0.000648532 | −0.00102984 | −0.00222199 |
| 63955 | Cables1 | 0.000427103 | −0.00485867 | 0 | −0.00182952 | −0.0007866 | −1.19E−05 | −0.00064613 |
| 14281 | Fos | −9.14E−05 | 0.004852144 | 0 | 0.003154537 | 0.002938306 | −0.00238451 | 8.46E−05 |
| 18125 | Nos1 | −0.00116422 | 0.004743415 | 0 | −0.00160979 | −0.00032316 | −0.00110304 | −0.00039174 |
| 78912 | Sp2 | 0.000304243 | 0.004709279 | 0 | 0.001744582 | 0.000965644 | 0.000186373 | 0.00062446 |
| 75563 | Dnali1 | −8.36E−05 | −0.0045938 | 0 | −0.0037951 | −0.00156167 | −5.59E−05 | 0 |
| 218442 | Serinc5 | −0.00075433 | −0.00443743 | 0 | −0.0017738 | −0.00158801 | −0.00046402 | −0.00160605 |
| 19201 | Pstpip2 | 0.001077793 | 0.00439248 | 0 | 0.001565206 | 0.001073606 | 0.001014365 | 0.00119508 |
| 66960 | Mlndy3 | 0.000232202 | −0.00429836 | 0 | −0.00068909 | −0.00054718 | −0.00021569 | −0.00012526 |
| 53600 | Tlmm23 | 4.60E−05 | −0.00425686 | 0 | −0.002397 | −0.00089615 | −0.00105626 | −0.00155912 |
| 67477 | Abhd15 | −0.00051216 | −0.00416152 | 0 | −0.00197305 | −0.00140549 | 0.000123694 | −0.00118552 |
| 27277 | Golga5 | −0.00013065 | 0.004048337 | 0 | 0.000384458 | 0.000600102 | 8.63E−05 | 0.000363293 |
| 242702 | Myom3 | −0.00109764 | 0.00404117 | 0 | 7.98E−05 | −9.46E−05 | −0.00071159 | 0.000255496 |
| 27207 | Rps11 | 0.000284523 | −0.00397936 | 0 | −0.0005033 | 0.00023437 | 0.000286406 | 0.000767754 |
| 271127 | Adamts16 | −0.00060685 | −0.00381576 | 0 | −0.00197945 | −0.00216718 | −0.00112408 | 0 |
| 114585 | D17H6353E | −0.00042431 | −0.0037604 | 0 | −0.00131059 | −0.00097556 | −0.00076707 | −0.00108573 |
| 15901 | Id1 | 0.001590254 | 0.003433649 | 0 | 0.00176705 | 0.001351064 | 0.000978593 | 0.002127373 |
| 194268 | 9930104L06Rik | −0.00087521 | 0.003351394 | 0 | 0.000360595 | 0.000316865 | −0.00065108 | 0.000404421 |
| 319899 | Dock6 | −0.00037516 | −0.0033075 | 0 | −0.00283724 | −0.0004042 | −0.00037039 | −0.00018484 |
| 22371 | Vwf | 0.001275784 | 0.003101822 | 0 | 0.005178003 | 0.003094999 | 0.001753882 | 0.004477262 |
| 329506 | Ctdspl2 | 0.000885645 | 0.003014487 | 0 | 0.002993975 | 0.001248408 | 0.00091733 | 0.001307999 |
| 243867 | Fbxo46 | 0.000194319 | 0.0029011 | 0 | −0.00083911 | −0.00057208 | −0.00022575 | −0.00078247 |
| 22139 | Ttr | 0.00074759 | 0.00280892 | 0 | 0.000276134 | −0.00205995 | −0.00070329 | −0.00367774 |
| 13726 | Emd | −0.00065077 | −0.00259443 | 0 | −0.00086872 | −0.00080074 | −0.00027786 | −0.00038281 |
| 74376 | Myo18b | 0 | −0.00245732 | 0 | −0.0028972 | −0.00164017 | 0 | 0 |
| 19385 | Ranbp1 | 0.001096785 | 0.002440116 | 0 | 0.001913996 | 0.001624043 | 0.000920621 | 0.001697143 |
| 231931 | Gimap6 | −0.00019474 | −0.00242723 | 0 | −0.00206035 | −0.00066489 | 4.18E−05 | −0.00060981 |
| 170790 | Mlc1 | 0.000109172 | 0.002330826 | 0 | 0.000192343 | 0.000599987 | 0 | 0 |
| 12155 | BmpZ | 0.000683344 | −0.00226341 | 0 | 0.000813544 | −8.30E−06 | 0.000222198 | −0.00035714 |
| 207728 | Pde2a | 0.000673182 | −0.002243 | 0 | −0.0017443 | 0.000808327 | 0.000157345 | 0.001872875 |
| 15560 | Htr2c | −0.00015136 | 0.002102078 | 0 | 0.00018708 | 0.00039896 | −0.00015537 | 0 |
| 215615 | Rnpep | −0.00034571 | −0.00198983 | 0 | −0.00421617 | −0.00160387 | 1.26E−05 | −0.00196888 |
| 98404 | Al597479 | 0.00014904 | 0.00189409 | 0 | 0.000820545 | 0.000677619 | −0.00010317 | 0.000353353 |
| 117589 | Asb7 | 0.000319069 | 0.001850447 | 0 | 0.00316627 | 0.001154924 | 0.00037092 | 0.001703036 |
| 214137 | Arhgap29 | 0.000602508 | −0.00182624 | 0 | −0.00174161 | −0.00024452 | 0.000225988 | 6.00E−05 |
| 230824 | Grhl3 | −0.00020272 | 0.001767371 | 0 | 0 | 0.000371213 | −3.36E−05 | 0 |
| 672511 | Rnf213 | 0.000189094 | 0.001593061 | 0 | 0.002762038 | 0.002061559 | −0.0002933 | 0.001755868 |
| 19735 | Rgs2 | 0.000645531 | −0.00158971 | 0 | −0.00356031 | −0.00145918 | 0.000541327 | −0.00314259 |
| 14183 | Fgfr2 | −7.60E−05 | 0.001574596 | 0 | 0.001672878 | 0.001208613 | 0.000294921 | 0.001067756 |
| 241226 | Itgs8 | −0.00132246 | −0.00154196 | 0 | −0.00284231 | −0.00212508 | −0.00091693 | −0.00211162 |
| 11363 | Acadl | 0.000283966 | 0.001535535 | 0 | 0.001328011 | 0.001098973 | 0.000248129 | 0.001335756 |
| 105439 | Slain1 | 0 | 0.001386847 | 0 | 0.000677938 | 0.000474955 | 0 | 0.000535175 |
| 23880 | Fyb | −0.00016719 | 0.001232868 | 0 | 0.003764326 | 0.001812607 | 0.000210205 | 0.002669498 |
| 14561 | Gdf11 | −0.00022661 | −0.00105127 | 0 | −0.00120081 | −0.00103695 | −0.00074905 | −0.00150274 |
| 12842 | Col1a1 | −0.00160657 | −0.00075207 | −0.02426017 | −0.00588826 | −0.00626356 | −0.00208211 | −0.00555303 |
| 140703 | Emid1 | −0.00098141 | −0.00071902 | 0 | −0.00105973 | −0.0023659 | −0.00170419 | −0.00139554 |
| 227753 | Gsn | 0.000469266 | −0.00070998 | 0 | −0.00230408 | −0.00096046 | 3.17E−05 | −0.00296323 |
| 17345 | Mki67 | 0.001597335 | −0.00060539 | 0 | −0.00034572 | −0.00021873 | 0.000397772 | −0.00225942 |
| 268977 | Ltbp1 | 0.000360427 | −0.00044323 | 0 | −0.00136221 | −0.00014306 | 7.33E−05 | −0.00098377 |
| 74513 | Neto2 | 0 | −0.00025134 | 0 | −0.00069541 | −0.0009525 | 0 | 0 |
| 235627 | Nbeal2 | −0.00058364 | 0.000140771 | 0 | 0.001818654 | 0.001369272 | −0.00049795 | 0.000633093 |
| 17132 | Maf | 0.000497048 | 0 | −0.06274904 | −0.00480151 | −0.00048871 | 0.001088165 | −0.00047074 |
| 67917 | Zcchc3 | −0.00051613 | 0 | −0.06172877 | −0.00420873 | −0.00081291 | −0.00047184 | −0.00131751 |
| 252972 | Tpcn1 | 0.000377475 | 0 | −0.05491568 | −0.00442079 | −0.00110028 | −0.00012304 | −0.0009512 |
| 17476 | Mpeg1 | 0.000208803 | 0 | 0.051844128 | 0.004809494 | 0.002425509 | −0.00024518 | 0.003536865 |
| 170742 | Sertad3 | −0.00059162 | 0 | 0.04372883 | 0.005300845 | 0.000649448 | −0.00051926 | 0.000898605 |
| 68701 | Ppp1r27 | 0 | 0 | 0.041086714 | 0 | 0.001116989 | 0 | 0 |
| 16519 | Kcnj3 | −0.00054004 | 0 | 0.040225409 | 0.003604684 | 0.001279189 | 0 | 0 |
| 16668 | Krt18 | −0.00090022 | 0 | 0.037930904 | 0.001480141 | 0.0002883 | −0.00094913 | −0.00019561 |
| 18762 | Prkcz | 0.000968688 | 0 | 0.03299119 | 0.003895379 | 0.002583242 | 0.000771685 | 0.00206495 |
| 60322 | Chst7 | −0.0014548 | 0 | 0.032331149 | 0.00418736 | −0.00042414 | −0.00076006 | −0.00096622 |
| 70779 | Prdm5 | −0.00091396 | 0 | −0.031927 | −0.00258259 | −0.00135939 | 0 | −0.00081984 |
| 68099 | Cibar1 | −0.00098757 | 0 | −0.03179603 | −0.00307495 | −0.00136071 | −0.00062524 | −0.0009352 |
| 14049 | Eya2 | −0.00053143 | 0 | 0.031401205 | 0.002611481 | 0.001660191 | 0 | 0.000928813 |
| 16497 | Kcnab1 | −0.00020641 | 0 | 0.03084549 | 0.002968833 | 0.001601041 | −0.00085107 | 0.000946621 |
| 56224 | Tspan5 | 0.000511386 | 0 | −0.03068035 | 6.30E−05 | −0.00047076 | 0.000763754 | 0.00172926 |
| 269016 | Sh3rf2 | −0.00012512 | 0 | 0.028945952 | −0.00089191 | 0.000447563 | −0.00024837 | −0.00024523 |
| 11722 | Amy1 | 0.000454053 | 0 | 0.028608873 | 0.004943345 | 0.00228748 | 0.000780216 | 0.002980785 |
| 14588 | Gfra4 | −0.00049577 | 0 | −0.02581773 | −0.00146348 | −0.00127433 | −0.00130517 | −0.00242069 |
| 212937 | Tifab | −0.00084573 | 0 | −0.02539785 | −0.00649249 | −0.00166484 | −0.00105566 | −0.00245789 |
| 239790 | Ostn | 0 | 0 | 0.025256797 | 0 | 0.001588761 | 0 | 0 |
| 19713 | Ret | −8.06E−05 | 0 | −0.02422053 | −0.00247581 | −0.00113008 | −0.00032779 | −0.00200895 |
| 67419 | Armh4 | 0 | 0 | 0.024084358 | 0.002365176 | 0.001295716 | 0 | 0.000635764 |
| 11815 | Apod | 0 | 0 | 0.023817249 | 0.006368235 | 0.004295836 | 0 | 0.00596701 |
| 329872 | Frem1 | −0.00069523 | 0 | −0.02176087 | 0.000483522 | −0.00052818 | −0.00017522 | −0.00064592 |
| 208151 | Tmem132b | 0 | 0 | 0.021514484 | 4.68E−05 | −7.40E−06 | 0 | 0 |
| 22436 | Xdh | 0.000834983 | 0 | 0.021173742 | 0.00566622 | 0.003217454 | 0.000449219 | 0.004558998 |
| 319765 | Igf2bp2 | −0.00037184 | 0 | −0.02109404 | −0.00467739 | −0.00356918 | −0.00065454 | −0.00311893 |
| 20196 | S100a13 | −0.00084284 | 0 | −0.02060234 | −0.00259557 | −0.0007552 | −0.00065364 | −0.00074653 |
| 170833 | Hook2 | −0.00183898 | 0 | 0.020093794 | 0.000883381 | 0.000874429 | −0.00139727 | 0.00096356 |
| 381148 | Prob1 | 9.84E−05 | 0 | −0.01999923 | 0.000203629 | −0.00062864 | −0.00021854 | −0.00106851 |
| 76980 | Ube2ql1 | −0.0011615 | 0 | 0.019844217 | 0.001337673 | 0.000836373 | −0.00070314 | 0.000980967 |
| 396184 | Flrt1 | −0.00012908 | 0 | −0.01965942 | −0.0019894 | −0.00060197 | 0.000581814 | −0.00039861 |
| 74596 | Cds1 | 4.16E−05 | 0 | 0.019037399 | 0.005221531 | 0.002351439 | −0.00018751 | 0.003227195 |
| 105722 | Ano6 | −0.00058726 | 0 | 0.018937106 | 0.001759074 | 0.000493253 | −2.04E−05 | 0.000414456 |
| 320997 | Cyp4f39 | 8.07E−05 | 0 | 0.018740208 | 0.001020403 | 0.000843678 | −0.00026984 | 0 |
| 13616 | Edn3 | −0.00040597 | 0 | −0.0186662 | −0.00077073 | −0.00124278 | −0.00075815 | −0.00276443 |
| 66714 | 4921524J17Rik | −0.00030708 | 0 | −0.01855131 | −0.00294671 | −0.00149347 | 0.000613001 | −0.00168342 |
| 110862 | Kcnq3 | 0.000345616 | 0 | 0.018206579 | 0.002795352 | 0.001363361 | 0.000346375 | 0.000868882 |
| 53311 | Mybph | 0.000347427 | 0 | 0.018169792 | 0.001433899 | 0.000304907 | 0 | 0 |
| 69065 | Chec1 | −0.00049591 | 0 | −0.01755688 | −0.00114542 | −0.00137037 | 0.000378054 | −7.66E−05 |
| 234577 | Cpne2 | 0.001521368 | 0 | 0.016730413 | 0.005136409 | 0.002317544 | 0.001741865 | 0.003623208 |
| 629147 | Ctxn3 | 0 | 0 | 0.015764156 | 0.001871315 | 0.00190644 | 0 | 0 |
| 209448 | Hoxc10 | −3.88E−05 | 0 | −0.01504759 | −0.00153927 | −0.00156833 | −0.00010034 | 0 |
| 12643 | Chad | 0.000153598 | 0 | −0.01485251 | −0.00292946 | −0.00222903 | 0.000637205 | −0.00321257 |
| 67141 | Fbxo5 | −0.00044802 | 0 | −0.0146367 | 0.001294897 | 0.000255784 | −0.00052243 | 0.000983722 |
| 230775 | Adgrb2 | 0.001724221 | 0 | 0.014287154 | 0.001344589 | 0.001546139 | 0.000936749 | 0 |
| 19288 | Ptx3 | 0.000519865 | 0 | −0.0141364 | −0.00361936 | −0.00096596 | 0 | 0 |
| 17392 | Mmp3 | −0.00118455 | 0 | 0.013982249 | 0.004038024 | 0.00238819 | −0.00126103 | 0.003633842 |
| 19038 | Pplc | −0.00046874 | 0 | −0.01354168 | −0.00116993 | −0.0022795 | −0.00022006 | −0.00191352 |
| 223332 | Ranbp3l | −0.00041872 | 0 | −0.01332471 | −0.00055445 | −0.00098832 | −0.00096473 | 0 |
| 70021 | Nt5dc2 | 0.001050718 | 0 | −0.01322602 | −0.00381917 | −0.00203113 | 0.000318803 | −0.00257134 |
| 320633 | Zbtb26 | 8.86E−05 | 0 | −0.01294384 | −0.0026339 | −0.00085366 | 0.000167856 | −0.00090894 |
| 11435 | Chrna1 | 0.00055059 | 0 | 0.012874997 | 0.001248133 | 0.001092622 | 0.000380567 | 0 |
| 107765 | Ankrd1 | 1.28E−05 | 0 | 0.012862547 | 0.003218697 | 0.00195903 | 0.000372174 | 0 |
| 170835 | Inpp5j | −0.00095427 | 0 | −0.0128059 | −0.0020645 | −0.00180607 | −0.00069177 | −0.00175514 |
| 74116 | Pi16 | 0.000357004 | 0 | −0.01274635 | −0.00196068 | −0.0009482 | 8.10E−05 | −0.001935 |
| 208501 | Ndufaf8 | −0.00042613 | 0 | −0.01246974 | −0.00439993 | −0.00100708 | −0.00059221 | −0.0015959 |
| 244853 | Nxpe4 | 0.001147062 | 0 | −0.01234869 | −0.0035135 | −9.67E−05 | −0.00033888 | −0.00203304 |
| 17528 | Mp2 | 6.71E−05 | 0 | −0.01229087 | −0.00058968 | −0.00142715 | 0.000371987 | 0 |
| 27260 | Plek2 | −0.00036146 | 0 | 0.012165668 | 0.003593205 | −1.28E−05 | −0.00032648 | 0.000261908 |
| 20271 | Scn5a | −4.16E−05 | 0 | 0.01198875 | 0.001799345 | 0.000799809 | 0 | 0 |
| 13717 | Eln | −0.00071479 | 0 | −0.01189724 | 3.54E−05 | −0.00302845 | −0.0016727 | −0.00221228 |
| 77963 | Hook1 | −0.00057537 | 0 | 0.011700279 | 0.001030639 | 0.001275152 | −0.00041629 | 0.001149295 |
| 233752 | Insc | 0.001716282 | 0 | −0.0116312 | 0.001591581 | −0.00032521 | 0.001132301 | 0.000353226 |
| 245537 | Nlgn3 | −2.32E−05 | 0 | −0.01150045 | −0.0009769 | −0.0005617 | −0.00018201 | 0 |
| 170720 | Card14 | −1.35E−05 | 0 | −0.01046393 | −0.00038628 | −8.18E−05 | 0.000373326 | 0 |
| 277743 | Fam131c | 0.000180194 | 0 | −0.01017105 | −0.00049976 | −0.00113485 | −0.0007192 | 0 |
| 83671 | Sytl2 | 0.000793922 | 0 | −0.01002195 | −0.0007484 | −2.72E−05 | 0.000488923 | −0.00128447 |
| 72634 | Tarkh | −0.00056862 | 0 | 0.009926766 | 0.001401676 | 0.001808707 | −8.58E−05 | 0.001909825 |
| 219228 | Pcdh17 | 0.00148068 | 0 | −0.00985288 | −0.0008455 | −0.00171832 | −0.00028015 | −0.00208493 |
| 14104 | Fasn | −0.00027049 | 0 | 0.00901968 | 0.001101885 | 0.000800118 | −0.00042522 | −0.00018007 |
| 382051 | Pdp2 | −0.00027266 | 0 | −0.00880233 | −0.00011497 | −0.000324 | −0.00117355 | −0.0008845 |
| 73707 | Gucy2g | 0.000127449 | 0 | 0.008634908 | 0 | 0.000516718 | 0.000222083 | 0 |
| 17906 | Myl2 | 0 | 0 | −0.00860165 | −0.00103599 | −0.00019551 | 0 | 0 |
| 230163 | Aldob | −0.00024482 | 0 | −0.0079202 | 0.000655435 | 0.000191907 | 0.000367943 | 0.001875423 |
| 20512 | Slc1a3 | −0.00115689 | 0 | −0.00788502 | −0.00020217 | −0.00142704 | −0.00072567 | −9.69E−05 |
| 20259 | Scin | 0.000168955 | 0 | 0.007757706 | 0.000115507 | 0.002652971 | 0.000366703 | 0.001885759 |
| 66425 | Pcp4l1 | −0.00136252 | 0 | −0.00744299 | 0.000939583 | −0.00018044 | −0.00186241 | −0.00018088 |
| 66402 | Sln | −0.00134429 | 0 | 0.007136422 | 0 | 0.00044721 | −0.00183034 | 0 |
| 18111 | Nnat | 2.71E−05 | 0 | 0.007131613 | 0.001817747 | 0.000506896 | −0.00038693 | 0 |
| 72309 | Tmem158 | −0.00015306 | 0 | 0.006948147 | 0.003296411 | 0.001637579 | 0.000619412 | 0.001834162 |
| 13009 | Csrp3 | −0.00089777 | 0 | −0.00692437 | −0.0019029 | −0.00078974 | 0.00104291 | 0.000939076 |
| 17879 | Myh1 | 0.000107962 | 0 | 0.006502928 | 0.001975798 | 0.000833377 | −0.00030079 | 0.001880783 |
| 75219 | Dusp18 | −0.00090535 | 0 | −0.00632656 | −0.00296456 | −0.00174282 | 0 | −0.00193115 |
| 16790 | Anpep | −0.00054213 | 0 | 0.006271657 | 0.000294836 | 0.00022223 | −0.00108026 | 0.000567744 |
| 101772 | Ano1 | −0.00016955 | 0 | 0.006245121 | 0.000130673 | 0.000155042 | 0.000408866 | 0.00059748 |
| 20379 | Sfrp4 | 0.000235621 | 0 | −0.00599595 | −0.00113768 | −0.00052309 | 0.000122939 | −0.0006233 |
| 27273 | Pdk4 | 0.001455804 | 0 | −0.00592615 | 0.000332257 | 0.000741272 | 0.000964338 | 0.001620266 |
| 13723 | Emb | −0.00015273 | 0 | 0.005788839 | −0.00321844 | −0.00056542 | −0.00045127 | −0.00138154 |
| 20464 | Sim1 | 0.000389156 | 0 | 0.005743115 | −0.00106983 | 0.00030415 | 0.000144154 | 0 |
| 277898 | Slc15a5 | 0.000180474 | 0 | 0.005601245 | 0.000571571 | −0.00014711 | −0.00050018 | 0 |
| 26878 | B3galt2 | −0.00108269 | 0 | 0.005266561 | −0.00032824 | −2.21E−05 | 0 | 0.000339545 |
| 252864 | Dusp15 | 0 | 0 | −0.0052463 | −0.00258366 | −0.00079489 | 0 | 0 |
| 68797 | Pdgfrl | −8.77E−05 | 0 | −0.00516107 | −0.00386776 | −0.00231579 | −0.00132146 | −0.0041653 |
| 72709 | C1qtnf6 | −0.00158554 | 0 | −0.00511261 | −0.00473814 | −0.00376597 | −0.00127836 | −0.0041807 |
| 12576 | Cdkn1b | 0.000829876 | 0 | −0.00508741 | −0.00184854 | −0.00122533 | 0.000255595 | −0.00165597 |
| 18053 | Ngfr | −0.00064404 | 0 | 0.004963109 | 0.003337243 | 0.002295856 | −0.00022434 | 0.003676563 |
| 17153 | Mal | −0.00109168 | 0 | −0.00476609 | −0.00083316 | −0.00043168 | −0.00092721 | −0.00072694 |
| 19124 | Procr | −0.00132631 | 0 | −0.00448406 | −0.00391358 | −0.00172726 | −0.00123433 | −0.00189523 |
| 109270 | Prr5 | 0.000711022 | 0 | −0.00445305 | 4.11E−05 | −0.0004408 | 0.000354287 | −0.00103473 |
| 229599 | Ciart | −0.00123081 | 0 | 0.004438346 | 0.00208239 | 0.001165261 | 0.000132356 | 0.0008765 |
| 71839 | Osgin1 | 0.000360469 | 0 | −0.0043558 | −0.00138582 | 0.001072932 | 0.000114483 | 0.001395624 |
| 70134 | Misp3 | 0 | 0 | −0.00423 | 0 | −0.00062736 | 0 | 0 |
| 20195 | S100a11 | −0.00132786 | 0 | −0.00410391 | −0.00274431 | −0.00120605 | −0.00037065 | −0.00092876 |
| 16846 | Lep | 0.000302311 | 0 | −0.00400087 | 0.000157702 | 4.64E−05 | 2.48E−05 | 0 |
| 11941 | Atp2b2 | −0.00071414 | 0 | 0.003768355 | −0.00098407 | 4.38E−05 | −0.00134473 | −0.00124764 |
| 12575 | Cdkn1a | 0.001318001 | 0 | 0.003671708 | 0.00430124 | 0.004231397 | 0.003041043 | 0.005304553 |
| 17883 | Myh3 | 0.000639359 | 0 | 0.003524679 | 0 | 0.000813907 | 0 | 0 |
| 216188 | Aldh1l2 | 0.00036675 | 0 | −0.0034482 | −0.00167191 | −0.00145248 | 0.000697171 | −0.0005349 |
| 56437 | Rrad | 0 | 0 | 0.003411973 | 0.000900537 | 0.000637878 | 0 | −6.43E−05 |
| 67044 | Higd2a | −0.00139903 | 0 | −0.00298123 | −0.00344352 | −0.00072428 | −0.00099026 | −0.00043432 |
| 140493 | Kcnn3 | 0.000510618 | 0 | 0.002979794 | −0.00203407 | 0.000397973 | −0.000197 | −0.00147048 |
| 50877 | Neu3 | −0.00093269 | 0 | −0.00278869 | −0.00348829 | −0.00043225 | −0.00163145 | −0.00158577 |
| 225164 | Mib1 | 0.001103679 | 0 | 0.00268273 | 0.002908965 | 0.001160036 | 0.000360658 | 0.001032701 |
| 319455 | Pld5 | 0 | 0 | 0.002327737 | 0.000882201 | 0.00015218 | 0 | 0 |
| 83922 | Cep41 | −0.0004148 | 0 | −0.00228455 | 0.000227008 | −0.00053778 | −3.09E−05 | −0.00065598 |
| 319197 | Gpr4 | −0.00027109 | 0 | −0.00220279 | −0.00342727 | −0.00143036 | −0.00035398 | −0.00195424 |
| 81904 | Cacng7 | 0 | 0 | −0.00180008 | −0.00212415 | −0.00015986 | 0 | 0 |
| 109663 | Hoxc11 | 0 | 0 | −0.00178085 | 0 | 0 | 0 | 0 |
| 14555 | Gpd1 | 0.000841526 | 0 | 0.001747804 | 0.00193695 | 0.001152363 | 0.000705781 | 0.002026828 |
| 213469 | Lgi3 | 0.000218482 | 0 | −0.00172558 | −0.0014677 | −0.00045516 | −0.00022997 | 0 |
| 67096 | Mmachc | 0.000364305 | 0 | −0.00154228 | 0.000352926 | 0.000873121 | 0.000262285 | 0.000842062 |
| 101359 | Prrt4 | 0 | 0 | 0.001540706 | 0.001193863 | 0.001032556 | 0 | 0 |
| 20692 | Sparc | −0.00131802 | 0 | −0.00153494 | −0.00435017 | −0.00384582 | −0.00135337 | −0.00485857 |
| 110082 | Dnah5 | 0 | 0 | −0.00143398 | −0.00115029 | −0.00086992 | 0 | 0 |
| 20422 | Sem1 | −0.00027085 | 0 | −0.00138637 | −0.00123182 | −0.00083708 | −0.00035977 | −0.00081723 |
| 329252 | Lgr6 | 0.000116904 | 0 | −0.00133581 | 0.001233354 | 3.59E−05 | −0.0001519 | 0 |
| 16367 | Irs1 | 0.00152957 | 0 | −0.00124611 | 0.001042437 | 8.17E−05 | 0.000365732 | −0.00046083 |
| 76376 | Slc24a2 | 0 | 0 | 0.00101924 | 0.00160063 | 0.00081453 | 0 | 0 |
| 66337 | Fam229b | 6.53E−05 | 0 | −0.00093343 | −0.00060702 | 8.14E−05 | 0.000237742 | 1.40E−05 |
| 13011 | Cst7 | 0.000821263 | 0 | 0.000910037 | 0.011791846 | 0.005165963 | 0.000670249 | 0 |
| 104886 | Rab15 | −6.02E−05 | 0 | −0.00084514 | −0.00269287 | −0.00094925 | −0.00032349 | −0.00034177 |
| 13107 | Cyp2f2 | −0.00102857 | 0 | 0.000833677 | 0.004377725 | 0.001382705 | −0.00052304 | 0.002551991 |
| 16005 | Igfals | ·0.0010132 | 0 | −0.00076976 | 0.000189861 | 0.00050965 | 0.000775738 | 3.98E−05 |
| 208164 | Fam180a | 0.00064526 | 0 | 0.000629369 | 0.002671143 | 0.00063351 | 0.000690743 | 0 |
| 74748 | Slamf8 | −0.0002792 | 0 | 0.000579724 | 0.000686093 | 0.001566013 | −0.00026695 | 0 |
| 14311 | Cidec | −0.00054136 | 0 | −0.00055332 | −0.0036925 | −0.00238461 | 0.000886334 | −0.00184802 |
| 15545 | Kera | 0.001034465 | 0 | −9.49E−05 | 0 | −0.00056791 | 0.000561194 | 0 |
| 16019 | Ighm | 0 | 0 | 0 | 0.009067107 | 0.004417439 | 0 | 0 |
| 14080 | Fabp1 | 0.001079331 | 0 | 0 | 0.008254862 | 0.000816766 | −0.00024042 | −0.00081573 |
| 20716 | Serpina3n | −0.00044507 | 0 | 0 | 0.008236361 | 0.001689782 | −0.00099118 | 0.002327255 |
| 16411 | Itgax | 0.001737739 | 0 | 0 | 0.007798921 | 0.003111264 | 0.001416316 | 0.004178341 |
| 320360 | Rlc3 | 0.001852914 | 0 | 0 | 0.007577647 | 0.003842135 | 0.001705886 | 0.005469014 |
| 14619 | Gjb2 | 0.000171389 | 0 | 0 | −0.00712806 | −0.00082587 | 0.001065531 | −0.00045158 |
| 76293 | Mfap4 | −0.00036262 | 0 | 0 | −0.0068986 | −0.00351301 | −0.00131711 | −0.00530944 |
| 12843 | Col1a2 | −0.00143731 | 0 | 0 | −0.00648065 | −0.00397935 | −0.00183567 | −0.00576909 |
| 12869 | Cox8b | 0 | 0 | 0 | 0.006365658 | 0.001489032 | 0 | 0 |
| 16956 | Lpl | 0.00163021 | 0 | 0 | 0.006353489 | 0.001511421 | −0.00113778 | 0.000507932 |
| 23833 | Cd52 | 0.00039632 | 0 | 0 | 0.006327487 | 0.002120708 | 0.000799941 | 0.00349263 |
| 12484 | Cd24a | −0.00117875 | 0 | 0 | −0.00624504 | −0.00195653 | 1.38E−05 | −0.00274387 |
| 227485 | Cdh19 | 0 | 0 | 0 | 0.006096335 | 0.002318667 | 0.002611933 | 0.003482467 |
| 434178 | Zfp141 | 0.000532035 | 0 | 0 | 0.005949108 | 0.001319598 | 0.000516097 | 0.00199375 |
| 106014 | Tafa5 | 0.000824178 | 0 | 0 | 0.005746665 | 0.00161807 | 0.001436119 | 0.003699397 |
| 15937 | Ier3 | 0.000465713 | 0 | 0 | 0.005721244 | 0.002235738 | 0.00017254 | 0.003604794 |
| 16768 | Lag3 | 8.15E−05 | 0 | 0 | 0.005654201 | 0.002067197 | 0.000570909 | 0.003092062 |
| 23943 | Esyt1 | 0.000415639 | 0 | 0 | 0.005643812 | 0.001514453 | 0.00042152 | 0.002284785 |
| 57262 | Retnla | 0 | 0 | 0 | −0.00556632 | −0.00200667 | 0 | 0 |
| 16601 | Klf9 | −7.69E−05 | 0 | 0 | −0.00555609 | −0.00179684 | 0.000161276 | −0.00122876 |
| 17085 | Ly9 | −0.00025233 | 0 | 0 | 0.005491461 | 0.001848405 | 0.000225477 | 0.004180739 |
| 12267 | C3ar1 | 0.000733842 | 0 | 0 | 0.005448899 | 0.002374317 | 0.00031783 | 0.002423038 |
| 14025 | Evl | −0.00051062 | 0 | 0 | −0.00542281 | −0.00277885 | −0.00019928 | −0.00367851 |
| 228094 | Cerkl | 0.00135581 | 0 | 0 | 0.005403495 | 0.001841049 | 0.00124063 | 0.00246369 |
| 22153 | Tubb4a | −0.00029009 | 0 | 0 | −0.00539644 | −0.00179358 | −0.00030902 | −0.00231446 |
| 20501 | Slc16a1 | 0.00026924 | 0 | 0 | 0.005379394 | 0.001774502 | 0.000401562 | 0.002394762 |
| 110557 | H2-Q6 | 0 | 0 | 0 | 0.005348031 | 0.001801057 | 0.000467463 | 0 |
| 17951 | Naip5 | 0.001204474 | 0 | 0 | 0.005346763 | 0.001280089 | 0.000400871 | 0.000901041 |
| 20666 | Sox11 | 0 | 0 | 0 | −0.00532048 | −0.00191824 | 0 | 0 |
| 15591 | Kl | −0.00035685 | 0 | 0 | −0.00531787 | −0.00120568 | −8.02E−05 | 0 |
| 12642 | Ch25h | −0.00044786 | 0 | 0 | 0.005314494 | 0.001788824 | −4.02E−05 | 0 |
| 214305 | Hhipl1 | 0.000428026 | 0 | 0 | 0.005311977 | 0.002112728 | 0.000154918 | 0.002475417 |
| 66548 | Adamtsl5 | 0.000855593 | 0 | 0 | 0.005298324 | 0.002011794 | 0.000366345 | 0.002133914 |
| 23972 | Papss2 | −0.00162128 | 0 | 0 | −0.00528031 | −0.00168084 | −0.00174081 | −0.00188704 |
| 54353 | Skap2 | 0.00103577 | 0 | 0 | 0.00525387 | 0.002650853 | 0.001231973 | 0.003836081 |
| 66102 | Cxcl16 | 0.001118375 | 0 | 0 | 0.005248007 | 0.00306196 | 0.00126442 | 0.002844699 |
| 59539 | Trnp1 | −4.71E−05 | 0 | 0 | 0.005226382 | 0.002073623 | −7.71E−05 | 0.003060631 |
| 74096 | Hvcn1 | 0.001180165 | 0 | 0 | 0.005197658 | 0.001721851 | 0.001832244 | 0.003181751 |
| 241230 | St8sia6 | −0.00074154 | 0 | 0 | 0.005170814 | 0.002662589 | −0.00040025 | 0.002855157 |
| 64453 | Zfp280b | 0.000509057 | 0 | 0 | 0.005122579 | 0.001383624 | 0.000426647 | 0.001028097 |
| 18295 | Ogn | −0.00093435 | 0 | 0 | −0.00507573 | −0.00156459 | −8.76E−06 | −0.00152867 |
| 53320 | Folh1 | −0.0009347 | 0 | 0 | 0.005064047 | 0.002502723 | −0.00072573 | 0 |
| 231991 | Creb5 | −0.0006541 | 0 | 0 | −0.00505254 | −0.00105824 | −0.00101065 | −0.00242334 |
| 14629 | Gclc | −0.00172453 | 0 | 0 | −0.00505231 | −0.00098921 | −0.00160801 | −0.00165916 |
| 407788 | BC051142 | 0 | 0 | 0 | 0.005038757 | 0.002101542 | −0.00055197 | 0 |
| 16997 | Ltbp2 | 0.000261448 | 0 | 0 | 0.005038305 | 0.002357488 | −0.00020123 | 0.00258597 |
| 237761 | Sowaha | −0.00073139 | 0 | 0 | −0.00499025 | −0.00153971 | 0 | 0 |
| 21753 | Tes | −0.00169446 | 0 | 0 | −0.0049835 | −0.00178754 | −0.00094137 | −0.00266894 |
| 140488 | Igf2bp3 | −0.00034109 | 0 | 0 | −0.00497086 | −0.00381069 | −0.00150274 | −0.00208163 |
| 17880 | Myh11 | −0.00193382 | 0 | 0 | −0.00496654 | −0.00205329 | −0.00195304 | −0.00214611 |
| 12268 | C4b | 0.000725559 | 0 | 0 | 0.004951507 | 0.002559577 | −0.00018037 | 0.002993014 |
| 12478 | Cd19 | 0.001039267 | 0 | 0 | 0.004946771 | 0.001567389 | 0.001200452 | 0.002309657 |
| 12797 | Cnn1 | −0.00025676 | 0 | 0 | −0.0049467 | −0.00202141 | 0.00043336 | −0.00222491 |
| 52428 | Rhpn2 | 0.001437046 | 0 | 0 | 0.004934189 | 0.001760957 | 0.000768206 | 0.001925661 |
| 75739 | Mpp7 | 0.000325564 | 0 | 0 | −0.00493263 | −0.0008703 | 0.000323598 | −0.00099106 |
| 76757 | Trdn | 0 | 0 | 0 | 0.004930691 | 0.002077397 | 0 | 0 |
| 16529 | Kcnk5 | −0.00012143 | 0 | 0 | −0.00492569 | −0.00063309 | 0.000759264 | −0.00258108 |
| 11816 | Apoe | 0.000264258 | 0 | 0 | 0.004909394 | 0.002786091 | 0.000364433 | 0.00391194 |
| 109032 | Sp110 | 0.000376287 | 0 | 0 | 0.004903308 | 0.002036623 | 0.000107752 | 0.003206614 |
| 20292 | Ccl11 | −6.12E−05 | 0 | 0 | 0.004893038 | 0.001524453 | −0.0006255 | 0.000437104 |
| 20897 | Stra6 | 7.57E−05 | 0 | 0 | 0.004874233 | 0.001852869 | 0.000299803 | 0.004237533 |
| 72097 | Cracdl | 0 | 0 | 0 | 0.004866938 | 0.001537613 | 0 | 0 |
| 216864 | Mgl2 | 0 | 0 | 0 | −0.00485899 | −0.00164372 | 0 | −0.00279226 |
| 242691 | Gpatch3 | 0.000509575 | 0 | 0 | 0.004849785 | 0.000613194 | 0.000484296 | 0.001740606 |
| 71829 | Ddi1 | 0 | 0 | 0 | 0.00484426 | 0.000853242 | 0 | 0 |
| 243653 | Clec1a | −0.00057716 | 0 | 0 | 0.004836731 | 0.001664552 | −0.00046161 | 0.002406054 |
| 56838 | Ccl28 | −3.59E−05 | 0 | 0 | −0.00483061 | −0.00093977 | −1.84E−05 | 0 |
| 239318 | Plcxd3 | 0 | 0 | 0 | 0.004803223 | 0.001993377 | 0 | 0 |
| 233405 | Vps33b | −0.00154435 | 0 | 0 | −0.00479451 | −0.0010975 | −0.00088441 | −0.00135891 |
| 13004 | Ncan | −0.00082299 | 0 | 0 | −0.00478341 | −0.00141759 | −0.00105759 | −0.00133456 |
| 11537 | Cfd | −0.00050326 | 0 | 0 | −0.00475557 | −0.0017398 | 0.002423401 | −0.00098181 |
| 215436 | Slc35e3 | 0.001136567 | 0 | 0 | 0.004748595 | 0.001334724 | 0.00076086 | 0.001358382 |
| 12262 | C1qc | 0.001244282 | 0 | 0 | 0.004741143 | 0.002974144 | 0.000386937 | 0.002641667 |
| 24108 | Ubd | 0.001745767 | 0 | 0 | 0.004738306 | 0.003320997 | 0.001312397 | 0 |
| 20564 | Slit3 | 0.000672028 | 0 | 0 | 0.004738293 | 0.002724471 | 0.000484227 | 0.00283006 |
| 20307 | Ccl8 | −0.0009286 | 0 | 0 | 0.004733647 | 0.002146042 | 0.000105112 | 0 |
| 93885 | Pcdhb14 | 0.000188266 | 0 | 0 | 0.004729659 | 0.001702648 | 2.44E−05 | 0 |
| 27053 | Asns | 0.000302819 | 0 | 0 | 0.004727048 | 0.001188643 | 9.00E−05 | 0.002380013 |
| 69480 | Ttc9 | 0 | 0 | 0 | 0.004715642 | 0.001778191 | 0 | 0 |
| 17329 | Cxcl9 | −0.00063802 | 0 | 0 | −0.00470945 | −0.00051172 | −0.00180049 | −0.00400336 |
| 71519 | Cyp2u1 | 0.000598732 | 0 | 0 | 0.004691532 | 0.001835188 | −0.00060985 | 0.001856011 |
| 12826 | Col4a1 | −0.00185862 | 0 | 0 | −0.00465766 | −0.00349519 | −0.00200674 | −0.00433275 |
| 53356 | Eif3g | 0.000240039 | 0 | 0 | 0.004656831 | 0.001321805 | −9.69E−06 | 0.001551631 |
| 56429 | Dpt | −0.00101363 | 0 | 0 | −0.00462087 | −0.00254075 | −0.00199721 | −0.00388237 |
| 68969 | Eif1b | 0.001013314 | 0 | 0 | 0.0046137 | 0.001811861 | 0.000950997 | 0.002365879 |
| 74202 | Fblim1 | 0.001494966 | 0 | 0 | 0.004612433 | 0.001073183 | 0.000839636 | 0.001789728 |
| 213417 | Klhdc8a | −0.00106144 | 0 | 0 | 0.004598202 | 0.00178863 | −0.0012496 | 0.00180062 |
| 237711 | Eml6 | 0.001033534 | 0 | 0 | 0.004597455 | 0.001206867 | 0.001268509 | 0.001795397 |
| 12819 | Col15a1 | 3.16E−09 | 0 | 0 | −0.00458522 | −0.00255814 | 0.000134388 | −0.00305302 |
| 76654 | Upp2 | −0.0015917 | 0 | 0 | −0.0045819 | −0.00166821 | −6.11E−05 | −0.00294004 |
| 58916 | Myot | 0 | 0 | 0 | 0.004568753 | 0.001907069 | 0 | 0 |
| 18008 | Nes | 0.000649058 | 0 | 0 | 0.004558267 | 0.001564956 | 0.000495618 | 0.002671387 |
| 217705 | Fam161b | 0.001098337 | 0 | 0 | 0.004538923 | 0.00149869 | 0.000642001 | 0.001048296 |
| 101314 | Brk1 | 0.001092858 | 0 | 0 | 0.004519651 | 0.00058823 | 0.000865341 | 0.000871208 |
| 18712 | Pim1 | 0.00127126 | 0 | 0 | 0.00451775 | 0.001107432 | 0.000593619 | 0.002820123 |
| 71984 | Sars2 | −0.0012998 | 0 | 0 | −0.00451634 | −0.0018277 | −0.00087164 | −0.00215393 |
| 23796 | Aplnr | −3.30E−05 | 0 | 0 | −0.00451223 | −0.00283387 | 3.17E−05 | −0.00457394 |
| 104601 | Mycbpap | −0.00085548 | 0 | 0 | −0.00449532 | −0.00174469 | −0.00038619 | −0.00243205 |
| 23954 | Nek3 | −0.0014091 | 0 | 0 | −0.0044951 | −0.00174224 | −0.00126687 | −0.00214635 |
| 72114 | Zbed3 | 0.001347822 | 0 | 0 | −0.00449099 | −0.00155952 | 0.001360374 | −0.00183931 |
| 58243 | Nap1l5 | 0.000424316 | 0 | 0 | 0.004471677 | 0.001495252 | 0.000638793 | 0.001858669 |
| 239554 | Foxred2 | 0.00021233 | 0 | 0 | 0.004469208 | 0.002261759 | −0.00101273 | 0.002180517 |
| 16142 | Iglv1 | 0 | 0 | 0 | 0.004451888 | 0.002004446 | 0 | 0 |
| 57339 | Jph1 | 0.000604858 | 0 | 0 | 0.004437048 | 0.001452302 | 0.001250153 | 0.002158238 |
| 17110 | Lyz1 | 0.001532121 | 0 | 0 | 0.004433218 | 0.000617611 | 0.002136271 | 0.001262337 |
| 75311 | 4930550C14Rik | −0.00081947 | 0 | 0 | −0.00442833 | −0.00205768 | −0.00055981 | 0 |
| 24099 | Tnfsf13b | 0.000147191 | 0 | 0 | 0.004428145 | 0.002108935 | 0.000613498 | 0.001599159 |
| 71781 | Slc16a14 | 0 | 0 | 0 | −0.0044277 | −0.00138231 | 0 | 0 |
| 68404 | Nrn1 | 0.000729612 | 0 | 0 | 0.00442385 | 0.001542283 | 0.000647431 | 0.003527411 |
| 404634 | Macroh2a2 | −0.00045388 | 0 | 0 | −0.00438971 | −0.00201644 | −0.00053982 | 0 |
| 18985 | Pouzaf1 | 4.71E−05 | 0 | 0 | 0.004377189 | 0.001933202 | 0.001214545 | 0.003846306 |
| 20183 | Rxrg | 0.00139107 | 0 | 0 | 0.004375268 | 0.001360568 | 0.001455144 | 0.002287522 |
| 74199 | Vlt | 0 | 0 | 0 | 0.004368558 | 0.001712229 | 0 | 0 |
| 12182 | Bst1 | −0.00054378 | 0 | 0 | −0.0043665 | −0.0017549 | 8.75E−05 | −0.00036398 |
| 320502 | Lmod3 | 0 | 0 | 0 | −0.00436123 | −0.00112886 | 0 | 0 |
| 232987 | B9d2 | −0.0018377 | 0 | 0 | −0.00435415 | −0.0008952 | −0.00058203 | −0.00119571 |
| 15211 | Hexa | 0.00103092 | 0 | 0 | 0.004347764 | 0.00146791 | 0.000700928 | 0.00101421 |
| 21949 | Tnfsf8 | −0.00063774 | 0 | 0 | 0.004341952 | 0.001092636 | −0.00022143 | 0 |
| 238328 | Vash1 | −0.00125057 | 0 | 0 | −0.00433247 | −0.00233908 | −0.00109175 | −0.0031815 |
| 219189 | Vwa8 | 0.000500096 | 0 | 0 | 0.004318864 | 0.001166747 | −4.23E−05 | 0.001846308 |
| 56248 | Ak3 | −0.00135784 | 0 | 0 | −0.0043076 | −0.00125689 | −0.00102404 | −0.00196959 |
| 224691 | Zfp472 | −0.00071064 | 0 | 0 | −0.00430413 | −0.00124167 | −0.00027118 | −0.00120124 |
| 12508 | Cd53 | 0.001671015 | 0 | 0 | 0.004303301 | 0.001112686 | 0.001423521 | −0.00041126 |
| 52276 | Cdca8 | −0.00116682 | 0 | 0 | −0.00428174 | −0.00168017 | −0.00124105 | −0.0032958 |
| 233064 | Wdr62 | −0.00130026 | 0 | 0 | −0.00427878 | −0.0014205 | −0.00106745 | −0.00232424 |
| 546611 | Klhl33 | 0 | 0 | 0 | −0.00427187 | −0.00143007 | 0 | 0 |
| 12982 | Csf2ra | −0.00124174 | 0 | 0 | −0.00425991 | −0.00088393 | −0.00121936 | −0.00130906 |
| 68867 | Rnf122 | −0.00098318 | 0 | 0 | −0.00424298 | −0.0022652 | −0.00073981 | −0.00383936 |
| 434341 | Nlrc5 | −0.00042811 | 0 | 0 | 0.004233628 | 0.00324028 | −0.00018355 | 0.004831548 |
| 58859 | Efemp2 | 0.000871758 | 0 | 0 | 0.004225582 | 0.001584171 | 0.001404529 | 0.002444437 |
| 15507 | Hspb1 | −0.00051103 | 0 | 0 | −0.00422538 | −0.00128056 | −0.00054408 | −0.00135723 |
| 98256 | Kmo | −0.00085633 | 0 | 0 | −0.00422471 | −0.00216647 | −0.00049518 | −0.00295699 |
| 114716 | Spred2 | −0.00149122 | 0 | 0 | −0.00422065 | −0.0015137 | −0.00077246 | −0.00104393 |
| 16009 | Igfbp3 | −0.00153565 | 0 | 0 | −0.0042195 | −0.0014365 | −0.00097102 | −0.00133881 |
| 19260 | Ptpn22 | 0.001421613 | 0 | 0 | 0.004214724 | 0.002327018 | 0.001048674 | 0.00213869 |
| 16185 | Il2rb | −0.00110421 | 0 | 0 | −0.00420818 | −0.00154583 | −0.00122225 | −0.00324875 |
| 67158 | Sft2d3 | 0.000515628 | 0 | 0 | 0.004198744 | 0.000864243 | 0.000136498 | 0.001096571 |
| 93882 | Pcdhb11 | 0.000303676 | 0 | 0 | 0.004183426 | 0.001066565 | 1.21E−05 | 0 |
| 14605 | Tsc22d3 | −0.00065239 | 0 | 0 | −0.00418304 | −0.00176435 | −0.00090824 | −0.00252524 |
| 75472 | Cfap126 | −0.00149852 | 0 | 0 | −0.00418124 | −0.00093583 | −0.00027715 | −0.00104048 |
| 70536 | Qpct | 0.001524184 | 0 | 0 | 0.004179377 | 0.000885781 | 0.000901723 | 0.000696729 |
| 72572 | Spats2 | 0.001011751 | 0 | 0 | 0.004171403 | 0.000936464 | 0.000441405 | 0.001211806 |
| 19218 | Ptger3 | 0.000960418 | 0 | 0 | 0.00416967 | 0.001606018 | 0.00049419 | 0.001732458 |
| 100034361 | Mfap1b | 0.001050781 | 0 | 0 | 0.00416559 | 0.000988422 | 0.000609241 | 0.001268228 |
| 218820 | Zfp5O3 | 0.001452798 | 0 | 0 | 0.004152141 | 0.001712095 | 0.000687811 | 0.002045679 |
| 20474 | Slx4 | 0 | 0 | 0 | 0.004150386 | 0.001706048 | 0 | 0.001662526 |
| 15476 | Hs3st1 | 0.000247756 | 0 | 0 | −0.00414033 | −0.00169263 | 0.000121715 | −0.0019219 |
| 73739 | Cby1 | −0.00042289 | 0 | 0 | −0.00413739 | −0.00093185 | −0.00018518 | −0.0011672 |
| 69956 | Ptcd3 | −0.00067558 | 0 | 0 | 0.004132609 | 0.001208104 | −0.00074194 | 0.001908886 |
| 107528 | Magee1 | −0.000439 | 0 | 0 | 0.004127939 | 0.000870198 | −0.00074808 | 0.000554982 |
| 29857 | Mapk12 | 0.000711285 | 0 | 0 | 0.004115263 | 0.002432839 | 0.000380193 | 0.002321755 |
| 12902 | Cr2 | −0.00143611 | 0 | 0 | −0.0041135 | −0.00181255 | −0.00054524 | −0.00142658 |
| 268709 | Fam107a | 0.001596461 | 0 | 0 | 0.004110132 | 0.001894835 | 0 | 0 |
| 170574 | Sp7 | −0.00030662 | 0 | 0 | 0.004104608 | 0.000906943 | 0.000112799 | 0 |
| 23967 | Osr1 | −9.46E−05 | 0 | 0 | −0.00410184 | −0.00161394 | −0.0006367 | −0.0018723 |
| 16538 | Kcns1 | 0 | 0 | 0 | 0.004101196 | 0.001231567 | 0 | 0 |
| 21838 | Thy1 | 0.000265108 | 0 | 0 | 0.004098403 | 0.000486621 | 1.61E−06 | 0.003371427 |
| 14181 | Fgfbp1 | 0 | 0 | 0 | 0.004095656 | 0.000908082 | 0 | 0 |
| 213603 | Slc44a3 | −0.00121009 | 0 | 0 | −0.00409333 | −0.00156919 | −0.00247147 | −0.00424114 |
| 20618 | Sncg | −0.00020409 | 0 | 0 | 0.004080049 | 0.003512973 | −9.08E−05 | 0.004479781 |
| 59083 | Fetub | 0.000505708 | 0 | 0 | 0.004079969 | 0.001884592 | −0.00012232 | 0.00336252 |
| 74646 | Spsb1 | 0.001330248 | 0 | 0 | 0.004079441 | 0.001695386 | 0.001002717 | 0.001312597 |
| 56753 | Tacstd2 | 0 | 0 | 0 | 0.004077132 | 0.001362292 | 0 | 0 |
| 53313 | Atp2a3 | 0.001120268 | 0 | 0 | 0.004063466 | 0.001771647 | 0.001419011 | 0.002549218 |
| 67442 | Retsat | 0.000392841 | 0 | 0 | 0.004061362 | 0.001645289 | 0.000509398 | 0.00168589 |
| 16881 | Llg1 | −0.00042596 | 0 | 0 | 0.004048138 | 0.001361159 | −0.00034581 | 0.001245417 |
| 19009 | Pou6f1 | 0.001658817 | 0 | 0 | 0.004035828 | 0.002149878 | 0.000877401 | 0.002470832 |
| 18616 | Peg3 | −0.00012809 | 0 | 0 | −0.0040338 | −0.00171991 | 3.88E−05 | −0.00198711 |
| 106931 | Ketd1 | 0.001129219 | 0 | 0 | 0.004031158 | 0.00120265 | −0.00018197 | 0.001551114 |
| 54156 | Egfl6 | −0.0008744 | 0 | 0 | −0.00402202 | −0.00084649 | −0.00027499 | 0 |
| 13645 | Egf | 0.000623562 | 0 | 0 | −0.00401764 | −0.00099588 | −0.00092413 | −0.00213175 |
| 76108 | Rap2a | −0.0016099 | 0 | 0 | −0.0040094 | −0.00150678 | −0.00093385 | −0.00197124 |
| 66432 | Slc7a6os | −0.00097029 | 0 | 0 | −0.00400535 | −0.00080449 | −0.00050178 | −0.00086865 |
| 216616 | Efemp1 | 0.001423609 | 0 | 0 | 0.003999872 | 0.002481389 | 0.001076973 | 0.003403118 |
| 259097 | Olfr558 | 0 | 0 | 0 | −0.00399694 | −0.0017721 | 0 | 0 |
| 58994 | Smpd3 | −0.00161506 | 0 | 0 | −0.00399453 | −0.00213488 | −0.0022297 | −0.00244727 |
| 18159 | Nppc | −0.00038278 | 0 | 0 | −0.00398591 | −0.00087466 | 4.82E−06 | 0 |
| 14201 | Fhl3 | −0.00028079 | 0 | 0 | −0.00397806 | −0.00135353 | −0.00086937 | −0.00197176 |
| 70099 | Smc4 | −0.00094577 | 0 | 0 | −0.00397697 | −0.00236597 | −0.00104211 | −0.00271677 |
| 13136 | Cd55 | −0.00151056 | 0 | 0 | −0.00396935 | 0.001903 | −0.00099175 | −0.00262224 |
| 18817 | Plk1 | −0.00020581 | 0 | 0 | −0.00396552 | −0.00115217 | 6.32E−05 | 0 |
| 102502 | Pls1 | −0.00050581 | 0 | 0 | 0.003960723 | 0.001710726 | −0.00139929 | 0.001004872 |
| 67893 | Tmem86a | 0.000287236 | 0 | 0 | −0.00395205 | −0.00088522 | 0.000799834 | −0.00149465 |
| 71956 | Rnf135 | 0.000347708 | 0 | 0 | 0.003942889 | 0.000690739 | 0.00011294 | 0.000834766 |
| 21881 | Tkt | 0.000201688 | 0 | 0 | 0.003939711 | 0.001369987 | 0.000342351 | 0.001560008 |
| 54451 | Cpsf3 | −0.00036206 | 0 | 0 | −0.00393951 | −0.00076484 | −9.65E−05 | −0.00089767 |
| 15957 | Ifit1 | −0.00126604 | 0 | 0 | 0.003938017 | 0.001814218 | −0.00139466 | 0.00407937 |
| 72560 | Nealad2 | −0.00091293 | 0 | 0 | 0.003937179 | 0.000547488 | −0.00025412 | 0.0016809 |
| 328967 | Arhgef37 | −0.00076829 | 0 | 0 | 0.003935099 | 0.000605439 | −0.00079038 | 0.002894756 |
| 12702 | Socs3 | 0.001579977 | 0 | 0 | 0.003933195 | 0.001828622 | 0.000911116 | 0.003204641 |
| 213393 | Depp1 | −2.56E−05 | 0 | 0 | 0.003928915 | 0.001162464 | 0.000672352 | 0.002953284 |
| 242721 | Klhdc7a | 0.001507089 | 0 | 0 | 0.003920626 | 0.000971752 | 0.000136835 | 0.001133555 |
| 320825 | Samd5 | 0 | 0 | 0 | −0.00391522 | −0.00218477 | 0 | −0.00327783 |
| 72507 | Dzip1l | 0.001488294 | 0 | 0 | 0.003914268 | 0.001459221 | 0.000285341 | 0.001161305 |
| 66859 | Slc16a9 | −0.0007827 | 0 | 0 | −0.0039113 | −0.00118138 | 8.77E−05 | −0.0020714 |
| 16776 | Lama5 | 0.000405161 | 0 | 0 | 0.003911277 | 0.001444748 | 0.000352234 | 0.001803345 |
| 21922 | Clec3b | −0.00018967 | 0 | 0 | −0.0039037 | −0.00188445 | 0.000345503 | −0.00132104 |
| 80901 | Cxcr6 | 0.000148151 | 0 | 0 | 0.003893551 | 0.001800824 | 7.67E−05 | 0.001820785 |
| 11488 | Adam11 | −0.00133205 | 0 | 0 | −0.0038912 | −0.00087837 | −0.00141122 | −0.00042463 |
| 20514 | Slc1a5 | −0.00177331 | 0 | 0 | −0.00389032 | −0.00139416 | −0.00099591 | −0.00104433 |
| 384281 | Gatc | −0.0006421 | 0 | 0 | −0.00388964 | −0.00062669 | −0.00060341 | −0.00090461 |
| 229900 | Gbp7 | −0.00156823 | 0 | 0 | −0.00388771 | −0.00148525 | −0.00144319 | −0.00154309 |
| 29876 | Clic4 | −1.13E−05 | 0 | 0 | 0.003887332 | 0.001359343 | 2.38E−08 | 0.001916391 |
| 94214 | Spock2 | 5.02E−05 | 0 | 0 | 0.003880489 | 0.001087476 | 0.000158627 | 0.001987417 |
| 192198 | Lrrc4 | 0 | 0 | 0 | −0.0038758 | −0.00096184 | 0 | 0 |
| 22715 | Zfp57 | −0.00055226 | 0 | 0 | −0.0038754 | −0.00175026 | 0.00040079 | −0.00255111 |
| 75744 | Svip | 0.000650563 | 0 | 0 | 0.003873999 | 0.001344966 | 0.000528701 | 0.002268885 |
| 68743 | Anln | 0.000127371 | 0 | 0 | 0.003868696 | 0.001036213 | −0.0007379 | 0.000760432 |
| 207798 | Gramd1c | 0.001803542 | 0 | 0 | 0.003866757 | 0.00172571 | 0.000906031 | 0.002355102 |
| 212733 | Bicdl2 | −0.00073446 | 0 | 0 | −0.00385802 | −0.00092993 | −0.00039354 | 0 |
| 245902 | Ccdc15 | −0.00039274 | 0 | 0 | 0.003853902 | 0.001081225 | 0.000220117 | 0.002193215 |
| 12816 | Col12a1 | −0.00092675 | 0 | 0 | −0.00385353 | −0.00182852 | −0.00110397 | −0.00287864 |
| 66983 | Zfp830 | 0.000205431 | 0 | 0 | −0.00385137 | −0.00115517 | −0.00016578 | −0.00196937 |
| 110135 | Fgb | 0.00072582 | 0 | 0 | 0.003851217 | 0.000447081 | 1.55E−05 | 0 |
| 58909 | Fam13a | 0.000377823 | 0 | 0 | −0.00385053 | −0.00106391 | 0.00097398 | −8.30E−05 |
| 242939 | Cpz | −0.00106565 | 0 | 0 | 0.003846454 | 2.17E−05 | −0.00090494 | 0 |
| 18231 | Nxph1 | −0.00011842 | 0 | 0 | 0.00384248 | 0.002006179 | 0.000312433 | 0 |
| 14792 | Lpcat3 | −0.00130449 | 0 | 0 | −0.00384193 | −0.00175109 | −0.00163167 | −0.00287052 |
| 18979 | Pon1 | −3.75E−05 | 0 | 0 | −0.00383174 | −0.00188888 | 0.00062299 | 1.13E−05 |
| 207742 | Rnf43 | 0.001472605 | 0 | 0 | 0.003825521 | 0.001831724 | 0.000688793 | 0.001697784 |
| 170743 | Tlr7 | 0.00102057 | 0 | 0 | 0.003823491 | 0.00188756 | 5.53E−06 | 0.001867348 |
| 12224 | Klfs | 0 | 0 | 0 | 0.00382157 | 0.001561493 | 0 | 0.0015387 |
| 227377 | Farp2 | 0.000430434 | 0 | 0 | 0.003821059 | 0.001289786 | 0.001032664 | 0.002599741 |
| 105785 | Kdelr3 | 0.000404472 | 0 | 0 | −0.00382036 | −0.00225395 | 0.000382568 | −0.0033471 |
| 270210 | Zfp651 | −0.00141122 | 0 | 0 | −0.00381829 | −0.0013513 | −0.00072001 | −0.00170504 |
| 27226 | Pla2g7 | 0.000732242 | 0 | 0 | −0.00381722 | 0.001338732 | 0.001863081 | 0.000749652 |
| 223665 | CO30006K11Rik | 0.000574508 | 0 | 0 | 0.003813703 | 0.001307452 | 0.000816982 | 0.001483409 |
| 12483 | Cd22 | 0.000503155 | 0 | 0 | 0.003813398 | 0.002145216 | 0.000789737 | 0.002960977 |
| 54667 | Atp8b2 | −0.00125145 | 0 | 0 | −0.00381302 | −0.00123817 | −0.00055702 | −0.00191482 |
| 230837 | Asap3 | −0.00060259 | 0 | 0 | −0.00380243 | −0.00158139 | −0.00075003 | −0.00282807 |
| 102436 | Lars2 | −0.00145547 | 0 | 0 | 0.0037998 | 0.0018966 | −0.00112449 | 0.001958623 |
| 78892 | Crispld2 | −8.69E−05 | 0 | 0 | −0.00378676 | −0.00127925 | 0.000322986 | −0.00167925 |
| 234564 | Cesif | −7.95E−05 | 0 | 0 | −0.00378271 | −0.00197556 | −0.00050253 | −0.00308481 |
| 56226 | Espn | 0.000472134 | 0 | 0 | 0.003781035 | 0.002184515 | −0.00043945 | 0.002131416 |
| 68251 | Babam1 | 0.00011158 | 0 | 0 | 0.003775685 | 0.000672269 | −0.00019444 | 0.000735117 |
| 21386 | Tbx3 | 0.001847072 | 0 | 0 | 0.003768451 | 0.001185144 | 0.000762367 | 0.001923041 |
| 68667 | Trpm4 | 0.001482187 | 0 | 0 | 0.003763506 | 0.002254035 | 0.001374937 | 0.000808206 |
| 98267 | Stk17b | −0.00079911 | 0 | 0 | −0.00376233 | −0.00120549 | 0.000523204 | −0.00093512 |
| 16182 | Il18r1 | 0.001373156 | 0 | 0 | 0.003762148 | 0.001277552 | 0.000200308 | 0.000674259 |
| 68705 | Gtf2f2 | −0.0001519 | 0 | 0 | 0.003761652 | 0.000713629 | −0.00039038 | 0.000606763 |
| 330096 | Shisa3 | 0.000539484 | 0 | 0 | 0.003759809 | 0.001863637 | 0.000174216 | 0.002642416 |
| 54120 | Gipc2 | 0.000741132 | 0 | 0 | 0.003742341 | 0.001409342 | 0.000587086 | 0.001680679 |
| 13642 | Efnb2 | −0.00092174 | 0 | 0 | −0.00374086 | −0.00169641 | 5.27E−05 | −0.00140885 |
| 20202 | S100a9 | 0.001414471 | 0 | 0 | 0.003740207 | 0.002177724 | 0.003184038 | 0.003477985 |
| 69528 | 1700030J22Rik | 0.000510186 | 0 | 0 | 0.003738624 | 0.000721467 | 0 | 0 |
| 12950 | Hapln1 | −0.00011002 | 0 | 0 | 0.003735271 | 0.000230151 | 0.000238509 | 0 |
| 243725 | Ppp1r9a | −0.00074146 | 0 | 0 | −0.00372897 | −0.00195079 | −0.00145106 | −0.00323117 |
| 52466 | Slc46a1 | −0.00171137 | 0 | 0 | −0.00371591 | −0.00234144 | −0.00155004 | −0.00235437 |
| 98170 | Tmem132a | −0.00154046 | 0 | 0 | −0.00371006 | −0.00223133 | −0.00178864 | −0.00269336 |
| 75146 | Mfsd13a | 0.00141874 | 0 | 0 | 0.003693298 | 0.001919328 | 0.000501317 | 0.001771398 |
| 21833 | Thra | 0.000881573 | 0 | 0 | 0.003692067 | 0.000897643 | −0.00011214 | 0.001277885 |
| 72400 | Pinx1 | 0.000819158 | 0 | 0 | 0.003692043 | 0.000633439 | 0.00050224 | 0.000894089 |
| 384061 | Fndc5 | −0.00047465 | 0 | 0 | −0.00369039 | −0.00257672 | −0.00077332 | −0.00353369 |
| 52700 | Txndc17 | −0.0004558 | 0 | 0 | −0.00368721 | −0.00072022 | −0.000404 | −0.00035782 |
| 13482 | Dpp4 | −0.00038971 | 0 | 0 | −0.00367452 | −0.00169354 | −0.00054627 | −0.00229519 |
| 21892 | Tll1 | 0 | 0 | 0 | −0.00367348 | −0.0016475 | 0 | 0 |
| 66109 | Tspan13 | −0.0016462 | 0 | 0 | −0.00367183 | −0.00100737 | −0.00105962 | −0.00265052 |
| 17084 | Ly86 | −0.00023462 | 0 | 0 | 0.00366891 | 0.00127496 | −0.00014942 | 0.00175454 |
| 20280 | Scp2 | 0.001442251 | 0 | 0 | 0.003668428 | 0.001590258 | 0.000789041 | 0.001927775 |
| 331487 | Uprt | 0.000491132 | 0 | 0 | −0.00366751 | −0.00118575 | 0.000310044 | 0 |
| 218973 | Wdhd1 | 0.000488569 | 0 | 0 | 0.003663027 | 0.000710556 | −0.00077241 | 0.000448565 |
| 66694 | Uqcrfs1 | −0.00071023 | 0 | 0 | −0.00365959 | −0.00110269 | −0.00047126 | −0.0015477 |
| 64292 | Ptges | 0.000979419 | 0 | 0 | 0.003658382 | 0.000813113 | 0.000839723 | 0.00104431 |
| 17022 | Lum | 0.000653112 | 0 | 0 | 0.003658301 | 0.000575555 | 0.000407335 | 0.00122253 |
| 52906 | Ahi1 | 1.46E−05 | 0 | 0 | 0.003657045 | 0.00136006 | 0.000502521 | 0.00221159 |
| 545370 | Hmcn1 | 0.000328651 | 0 | 0 | 0.003655673 | 0.000125457 | −0.00034428 | 0.00020267 |
| 26432 | Plod2 | −0.00122159 | 0 | 0 | −0.00365091 | −0.00168521 | −0.00100919 | −0.00295132 |
| 103710 | Slc35e4 | −0.00026473 | 0 | 0 | −0.00364428 | −0.00054719 | 0.000406951 | −0.00077843 |
| 110948 | Hlcs | 0.001159386 | 0 | 0 | 0.003634235 | 0.001995967 | 0.000980345 | 0.002080744 |
| 13200 | Ddost | 2.64E−05 | 0 | 0 | 0.003628263 | 0.000551323 | 0.000698179 | 0.001368889 |
| 21687 | Tek | −0.00068376 | 0 | 0 | −0.00362628 | −0.00130072 | −0.000982 | −0.00235479 |
| 100201 | Tmem64 | 0.001432301 | 0 | 0 | −0.0036163 | −0.00014118 | 0.00116081 | −0.00072544 |
| 22004 | Tpm2 | −0.00111153 | 0 | 0 | −0.00361535 | −0.00071575 | 0.000177381 | −0.00062616 |
| 330286 | D630045J12Rik | −0.0007721 | 0 | 0 | −0.0036137 | −0.00105222 | −0.00077331 | −0.00195699 |
| 12876 | Cpe | 0.000652282 | 0 | 0 | 0.003612544 | 0.001280402 | 0.000612533 | 0.001928151 |
| 170442 | Bbox1 | 0.001042438 | 0 | 0 | 0.003607701 | 0.001248714 | 0.000806136 | 0.002331947 |
| 20677 | Sox4 | −0.00045816 | 0 | 0 | −0.00359402 | −0.00160688 | −0.00126881 | −0.00413895 |
| 100043272 | Inafm2 | 0.000937923 | 0 | 0 | 0.003592348 | 0.000923286 | 0.00037554 | 0.001052471 |
| 67847 | Sncaip | 0 | 0 | 0 | −0.00358858 | −0.0011259 | 0 | −0.001849 |
| 66058 | Tmem1768 | 0.001703961 | 0 | 0 | 0.003588477 | 0.001983695 | 0.00110362 | 0.002596761 |
| 67249 | Thcid19 | −0.00068057 | 0 | 0 | −0.00358242 | −0.0007528 | −0.00040219 | −0.00049104 |
| 56149 | Tamalin | −0.00017352 | 0 | 0 | −0.00357802 | −0.0007743 | −0.00012946 | −0.00050072 |
| 76915 | Mnd1 | −0.00082903 | 0 | 0 | 0.00357642 | 0.000493921 | −0.00040224 | 0.001440465 |
| 66311 | Cenpw | 0 | 0 | 0 | −0.0035682 | −0.00077555 | 0 | 0 |
| 54216 | Pcdh7 | 0.000696946 | 0 | 0 | 0.003560667 | 0.000688298 | −0.0001795 | 0.000469366 |
| 107476 | Acaca | 0.000914606 | 0 | 0 | 0.003556978 | 0.001187763 | −0.00014668 | 0.000700839 |
| 231986 | Jazf1 | 0 | 0 | 0 | −0.00355409 | −0.00164164 | 0 | −0.00386876 |
| 246707 | Emilin2 | −0.00188105 | 0 | 0 | −0.00354771 | −0.00052171 | −5.71E−05 | −9.11E−06 |
| 70750 | Kdsr | 0.000154652 | 0 | 0 | 0.003545185 | 0.000845743 | −5.93E−05 | 0.000942873 |
| 64817 | Svep1 | 0.000919491 | 0 | 0 | 0.003544093 | 0.002111493 | −8.07E−06 | 0.000713643 |
| 66953 | Cdca7 | −0.00038717 | 0 | 0 | −0.00354224 | −0.00158902 | −0.00033004 | −0.00120546 |
| 12804 | Cntfr | 0.001398376 | 0 | 0 | 0.003540121 | 0.001937606 | 0.001231351 | 0.002552685 |
| 270624 | Spin4 | −0.00057626 | 0 | 0 | −0.00353903 | −0.00104967 | −0.00024613 | 0 |
| 15368 | Hmox1 | −0.0001488 | 0 | 0 | −0.00353526 | −0.00039009 | 0.00084802 | −0.00060433 |
| 19378 | Aldh1a2 | 7.76E−06 | 0 | 0 | 0.003534179 | 0.001663193 | 0.000438464 | 0.002039183 |
| 22390 | Wee1 | 0.000498573 | 0 | 0 | −0.00353096 | −0.00074144 | 0.000216364 | −0.00170551 |
| 96957 | Tmem62 | −0.00105103 | 0 | 0 | −0.0035298 | −0.00178327 | −0.00036734 | −0.00133285 |
| 108078 | Olr1 | 0.000618893 | 0 | 0 | 0.003529705 | 0.000923198 | 0.000564852 | 0 |
| 233833 | Tnrc6a | −0.00030983 | 0 | 0 | −0.00352945 | −0.00063513 | 2.14E−05 | −0.00059143 |
| 20807 | Srf | 0.000159759 | 0 | 0 | 0.003528005 | 0.000291233 | −2.56E−05 | 0.000262815 |
| 15458 | Hpx | 0.0008849 | 0 | 0 | −0.00352698 | −0.00032403 | 0.000104374 | −0.00148439 |
| 17286 | Meox2 | −0.00074633 | 0 | 0 | −0.00352163 | −0.0011079 | −0.00119173 | −0.00102516 |
| 74735 | Trim14 | 0.001088486 | 0 | 0 | 0.003518697 | 0.000915193 | 0.000542965 | 0.001649835 |
| 12351 | Car4 | −0.001049 | 0 | 0 | −0.00351561 | −0.00182497 | −0.00113407 | −0.00224891 |
| 11553 | Adra2c | −0.00159465 | 0 | 0 | −0.00351551 | −0.00105728 | −0.00116498 | 0 |
| 11423 | Ache | 0.001224665 | 0 | 0 | 0.00351502 | 0.001928738 | −0.00018106 | 0.001880063 |
| 20364 | Selenow | 0.000206019 | 0 | 0 | 0.003513794 | 0.00089348 | 0.00086393 | 0.001469331 |
| 19285 | Cavin1 | −0.00018066 | 0 | 0 | 0.003511125 | 0.000988505 | −0.00039517 | 0.002023901 |
| 20494 | Slc1062 | −0.00058936 | 0 | 0 | −0.00351038 | −0.00122626 | 0.000163585 | 0 |
| 20612 | Siglec1 | −0.00025028 | 0 | 0 | 0.003507437 | 0.001654311 | −0.00109305 | 0.002098561 |
| 229906 | Gtf2b | 0.000130808 | 0 | 0 | 0.003506692 | 0.000653675 | −0.00018876 | 0.000697747 |
| 11810 | Apobec1 | 0.001615914 | 0 | 0 | 0.003506619 | 0.00184325 | 0.000295036 | 0.001481139 |
| 15505 | Hsph1 | −0.00122158 | 0 | 0 | −0.00350634 | −0.00191257 | −0.00146292 | −0.00191319 |
| 18792 | Plau | −0.00083784 | 0 | 0 | 0.003506203 | 0.001477695 | −0.00038425 | 0.001606397 |
| 11433 | Acp5 | −0.00043005 | 0 | 0 | −0.00350257 | −0.00118913 | −0.00023242 | −0.00208229 |
| 71436 | Flrt3 | −0.00031607 | 0 | 0 | −0.0035018 | −0.00113648 | −0.00053735 | −0.00344865 |
| 13058 | Cybb | 0.001479561 | 0 | 0 | 0.002980459 | 0.003061412 | 0.001581468 | 0.003637396 |
| 12654 | Chil1 | 0.00132546 | 0 | 0 | 0.002101533 | 0.002927697 | −0.00157188 | 0.000567641 |
| 14563 | Gdf5 | 0 | 0 | 0 | 0 | 0.002870797 | 0 | 0 |
| 18104 | Nqo1 | 0.001521306 | 0 | 0 | 0.003291676 | 0.002851969 | 0.001731156 | 0.002177324 |
| 14119 | Fbn2 | −0.00084117 | 0 | 0 | −0.00177895 | −0.00270317 | −0.00102643 | 0 |
| 20302 | Ccl3 | 0.000919644 | 0 | 0 | 0 | 0.00268869 | 0.001291994 | 0 |
| 26386 | Hsf4 | 0.000275739 | 0 | 0 | 0.000871876 | 0.002677524 | 0.000929239 | 0.001789269 |
| 21928 | Tnfaip2 | 0.000646876 | 0 | 0 | 0.003152165 | 0.002675492 | 0.000728769 | 0.003546147 |
| 27206 | Nrk | 0 | 0 | 0 | −0.00289842 | −0.0026044 | 0 | 0 |
| 68054 | Serpina1Z | 0.00115632 | 0 | 0 | 0 | 0.002598307 | 0.001084329 | 0 |
| 12406 | Serpinh1 | −0.00068453 | 0 | 0 | −0.00322299 | −0.00257658 | −0.00143244 | −0.00347533 |
| 13040 | Ctss | 0.000647592 | 0 | 0 | 0.002244502 | 0.002498762 | 0.000848772 | 0.003064246 |
| 14960 | H2-Aa | 0.00097567 | 0 | 0 | 0.000562708 | 0.002493782 | −0.00059771 | 0.000401903 |
| 94352 | Lox12 | −0.00147873 | 0 | 0 | −0.00179766 | −0.00246577 | −0.00135763 | 0 |
| 217203 | Tmem106a | 0.001185723 | 0 | 0 | 0.002283298 | 0.002453867 | 0.001637856 | 0.002130238 |
| 50530 | Mfap5 | −0.00016332 | 0 | 0 | −0.00325658 | −0.00245324 | 0 | 0 |
| 230379 | Acer2 | 3.81E−05 | 0 | 0 | 0.00324247 | 0.002412707 | 0.000944794 | 0.003257593 |
| 208691 | Eif5a2 | 0.000416412 | 0 | 0 | 0.003168718 | 0.002409001 | 0 | 0.00324864 |
| 14586 | Gfra2 | −0.00117586 | 0 | 0 | −0.00325279 | −0.00236614 | −0.00151547 | −0.00293802 |
| 22329 | Vcam1 | 0.001494989 | 0 | 0 | 0.003347246 | 0.002364811 | 0.001641687 | 0.003626121 |
| 12286 | Cacnala | 0.001019975 | 0 | 0 | 0.002305684 | 0.002363422 | 7.14E−05 | 0.001672499 |
| 30963 | Hacd1 | −0.00174781 | 0 | 0 | −0.00224597 | −0.00235815 | −0.00219085 | −0.00228521 |
| 64293 | Stk32b | −0.00035353 | 0 | 0 | 0.00260492 | 0.002348197 | −0.00022394 | 0 |
| 13527 | Dtna | 0.001558426 | 0 | 0 | 0.002675685 | 0.002338965 | 0.001267999 | 0.002276893 |
| 12870 | Cp | 0.001653298 | 0 | 0 | 0.00153786 | 0.002312692 | 0.001716324 | 0.001667175 |
| 171543 | Bmf | −0.0007238 | 0 | 0 | −0.00079441 | −0.00231067 | −3.97E−05 | −0.00077822 |
| 216725 | Adamts2 | −0.00111283 | 0 | 0 | −0.00295832 | −0.00229844 | −0.00103326 | −0.0024113 |
| 14128 | Fcer2a | 0.000254823 | 0 | 0 | −0.00226657 | −0.00229696 | −0.00022366 | −0.00303618 |
| 81799 | C1qtnf3 | 0.000553001 | 0 | 0 | −0.00161222 | −0.00225751 | 0.000129506 | −0.0027578 |
| 68709 | Cilp2 | −0.00011747 | 0 | 0 | −0.00254547 | −0.00225421 | −0.00046192 | 0 |
| 211550 | Tifa | 0.001854835 | 0 | 0 | 0.001775177 | 0.002236786 | 0.002143344 | 0.002619312 |
| 68591 | Mocos | −0.00070558 | 0 | 0 | 0.002294591 | 0.002218391 | −4.26E−05 | 0.002907183 |
| 17916 | Myo1f | 0.000458964 | 0 | 0 | 0.003298835 | 0.002211754 | 0.00115087 | 0.002732509 |
| 64918 | Bhmt2 | 0.000462542 | 0 | 0 | 0.003122719 | 0.002209232 | 0.000601361 | 0.002462268 |
| 18830 | Pltp | 0.00125965 | 0 | 0 | 0.001330948 | 0.002208103 | 0.001052578 | 0.001859999 |
| 78688 | Nol3 | 0.000296808 | 0 | 0 | 0.002693913 | 0.002194418 | 0.000251876 | 0.002065615 |
| 20377 | Sfrp1 | −0.00072071 | 0 | 0 | 0.002223694 | 0.002193406 | −0.00065173 | 0.002352514 |
| 22032 | Traf4 | −0.00010278 | 0 | 0 | −0.00293211 | −0.00218354 | −1.21E−05 | −0.00270193 |
| 14969 | H2-Eb1 | 0.00076928 | 0 | 0 | 0.000236756 | 0.00218002 | −0.00105687 | −0.00058362 |
| 69183 | C1qtnf2 | −0.00077004 | 0 | 0 | −0.00309816 | −0.00217622 | −0.00133594 | −0.00326071 |
| 246256 | Fcgr4 | 0.001120106 | 0 | 0 | 0.00211611 | 0.002174183 | 0.001758999 | 0.002679501 |
| 12828 | Col4a3 | 0.000215453 | 0 | 0 | −0.00344065 | −0.00217133 | 0.000890713 | −0.0025468 |
| 26399 | Map2k6 | −0.00052954 | 0 | 0 | −0.00248974 | −0.00214163 | −0.00035671 | −0.00226827 |
| 233733 | Galnt18 | −0.00124972 | 0 | 0 | −0.00262352 | −0.00214142 | −0.00069969 | −0.002354 |
| 14086 | Fscn1 | 0.000472961 | 0 | 0 | −0.0017257 | −0.00213278 | 0.000290921 | −0.00298396 |
| 15460 | Hr | −0.00052755 | 0 | 0 | −0.00250428 | −0.00213007 | 6.09E−05 | −0.00168426 |
| 22240 | Dpysl3 | 0.000821381 | 0 | 0 | −0.0032841 | −0.00212389 | 0.000577854 | −0.00225328 |
| 14962 | Cfb | 0.00121166 | 0 | 0 | 0.001257539 | 0.002115124 | 0.000965414 | 0.004452457 |
| 18478 | Pah | −0.00156645 | 0 | 0 | −0.00284039 | −0.00211286 | −0.00179029 | −0.00328504 |
| 13813 | Eomes | 0 | 0 | 0 | 0.003353911 | 0.002111765 | 0 | 0 |
| 12831 | Col5a1 | −0.00064679 | 0 | 0 | −0.00157352 | −0.00210344 | −0.00111124 | −0.00218469 |
| 18534 | Pck1 | −0.00029365 | 0 | 0 | −0.00185115 | −0.00208743 | −0.00032283 | −0.00386625 |
| 15370 | Nr4a1 | 0.001891407 | 0 | 0 | 0.00221017 | 0.002082355 | 0.001464544 | 0.001474067 |
| 338370 | Nalon | 0 | 0 | 0 | 0.00212538 | 0.002077966 | 0 | 0 |
| 246709 | Rgs13 | −0.00013279 | 0 | 0 | 0 | 0.002062241 | 0.000509733 | 0 |
| 233332 | Adamts17 | −0.00058668 | 0 | 0 | −0.00222128 | −0.00206073 | −0.00045721 | 0 |
| 69354 | Slc3884 | 0.000718219 | 0 | 0 | −0.00201229 | −0.00205431 | 0.000605359 | −0.00289356 |
| 15442 | Hpse | 0.00177098 | 0 | 0 | 0.00343326 | 0.002050447 | 0.001092111 | 0.003585361 |
| 16842 | Lef1 | −0.00126358 | 0 | 0 | −0.00222414 | −0.00204929 | −0.00089164 | 0 |
| 57914 | Crif2 | −0.00019438 | 0 | 0 | 0.002470974 | 0.00204366 | 0.000358163 | 0.002466591 |
| 22337 | Vdr | −0.00011871 | 0 | 0 | 0.00130977 | 0.00204014 | 0.000227582 | 0.00333928 |
| 22223 | Uchl1 | 0.000625393 | 0 | 0 | 0.003165497 | 0.002037393 | 0.000206017 | 0.002860699 |
| 22361 | Vnn1 | −0.00127695 | 0 | 0 | −0.00279434 | −0.00203535 | −0.00181687 | −0.00300391 |
| 209837 | Slc38a5 | −0.00145853 | 0 | 0 | −0.00283879 | −0.00203402 | −0.00268283 | −0.00301569 |
| 210719 | Mkx | −0.00016847 | 0 | 0 | 0.001310461 | 0.00202562 | −0.00096249 | 0.001159071 |
| 58998 | Nectin3 | −0.00067584 | 0 | 0 | −0.00323249 | −0.00202466 | −0.00075752 | −0.00225476 |
| 68774 | Ms4a6d | 0.001876335 | 0 | 0 | 0.003173596 | 0.002017099 | 0.001908259 | 0.001515199 |
| 109264 | Me3 | 0.000327346 | 0 | 0 | 0.00163409 | 0.002013631 | 0 | 0.001676809 |
| 83965 | Enpp5 | −0.00123111 | 0 | 0 | 0.001466104 | 0.002007689 | −0.00121346 | 0.001037744 |
| 110784 | Nr3c2 | 0.000654746 | 0 | 0 | 0.003433924 | 0.002006359 | 0.000522699 | 0.002352448 |
| 16912 | Psmb9 | −0.00046481 | 0 | 0 | 0.001303167 | 0.00200322 | 9.29E−05 | 0.001367838 |
| 108052 | Slc14a1 | −0.00051979 | 0 | 0 | 0.002554208 | 0.001998308 | 0 | 0 |
| 170677 | Cdhr1 | 0.001404447 | 0 | 0 | 0.001789613 | 0.001984401 | 0.001446949 | 0 |
| 16450 | Jag2 | 0.00170406 | 0 | 0 | 0.002580721 | 0.001980185 | 0.000873208 | 0.0018746 |
| 18073 | Nid1 | −0.00020545 | 0 | 0 | −0.00196744 | −0.00197783 | −3.95E−05 | −0.00117418 |
| 12775 | Ccr7 | 0.000284281 | 0 | 0 | −0.00344903 | −0.00197365 | −2.42E−05 | 0 |
| 17536 | Meis2 | 0.001011814 | 0 | 0 | 0.003006401 | 0.001969862 | 0.000796223 | 0.002588753 |
| 12696 | Cirbp | 0.000871147 | 0 | 0 | 0.001810796 | 0.00196494 | 0.001466331 | 0.002542289 |
| 17762 | Mapt | 0.001131085 | 0 | 0 | 0.001099081 | 0.001962334 | 0.001450166 | 0.00080384 |
| 16176 | Il1b | 0.001732767 | 0 | 0 | 0.001525113 | 0.001951834 | 0.00023265 | 0 |
| 19703 | Renbp | 0.00165649 | 0 | 0 | 0.002594006 | 0.00194803 | 0.001516974 | 0.001896944 |
| 21789 | Tfpi2 | −0.00145582 | 0 | 0 | −0.00346198 | −0.00194674 | −0.00097134 | −0.00257706 |
| 15904 | Id4 | 0.001892501 | 0 | 0 | 0.003036583 | 0.001946015 | 0.002563241 | 0.00232421 |
| 58226 | Cacna1h | 0.000609879 | 0 | 0 | 0.003109578 | 0.001945503 | 0.000123955 | 0.001594142 |
| 14127 | Fcer1g | 0.000799943 | 0 | 0 | 0.002720222 | 0.001940005 | 0.000467116 | 0.002070926 |
| 72290 | Lsm11 | −0.00040469 | 0 | 0 | −0.00112122 | −0.00193853 | −0.00054987 | −0.0014134 |
| 50908 | C1s1 | 0.001444307 | 0 | 0 | 0.002481021 | 0.001933704 | 0.001314079 | 0.002106837 |
| 22186 | Uba52 | 0.001053206 | 0 | 0 | 0.001730269 | 0.001932731 | 0.001882787 | 0.004125448 |
| 14130 | Fcgr2b | 0.000875137 | 0 | 0 | 0.001659186 | 0.001931149 | 0.000248344 | 0.000233137 |
| 21940 | Cd27 | −0.00047948 | 0 | 0 | −0.00328484 | −0.00193035 | −5.79E−05 | −0.00161379 |
| 327766 | Tmem26 | 0.00016854 | 0 | 0 | −0.00160232 | −0.00192848 | −0.00013405 | −0.00320195 |
| 20893 | Bhlhe40 | 0.000636841 | 0 | 0 | 0.00183728 | 0.001927385 | −8.80E−05 | 0.001760296 |
| 68087 | Dcakd | −1.74E−05 | 0 | 0 | −0.00232024 | −0.00192644 | 0.000236242 | −0.00172273 |
| 18822 | Plod1 | −0.00047814 | 0 | 0 | −0.00313416 | −0.00192533 | −0.00021862 | −0.00248205 |
| 12121 | Bicd1 | −0.00026094 | 0 | 0 | 0.001490562 | 0.001920573 | 0.000148344 | 0.001680545 |
| 270097 | Vat1l | 0 | 0 | 0 | −0.0030484 | −0.00191935 | 0 | 0 |
| 433470 | AA467197 | 0.000970068 | 0 | 0 | 0 | 0.001916875 | 0.001311179 | 0 |
| 13057 | Cyba | 0.001753445 | 0 | 0 | 0.002529713 | 0.001916685 | 0.001692375 | 0.003011333 |
| 22352 | Vim | 0.001714375 | 0 | 0 | 0.002766102 | 0.001914966 | 0.001517438 | 0.002688203 |
| 11801 | Cd5l | 0.001115918 | 0 | 0 | 0.001594088 | 0.00191185 | 0.000603289 | 0.00098304 |
| 18584 | Pde8a | −0.0011396 | 0 | 0 | 0.002227549 | 0.001906915 | −0.00064251 | 0.002497409 |
| 231162 | Cytl1 | −4.86E−05 | 0 | 0 | 0.00227054 | 0.001905975 | −0.00044637 | 0.001440279 |
| 18791 | Plat | 0.00131478 | 0 | 0 | 0.0030382 | 0.001905354 | −0.000677 | 0.000389403 |
| 18113 | Namt | −0.00046951 | 0 | 0 | −0.0012879 | −0.00189164 | −0.00024431 | −0.00205549 |
| 11746 | Anxa4 | 0.000835979 | 0 | 0 | 0.003157833 | 0.001887347 | 0.00050214 | 0.001177258 |
| 78134 | Lpar4 | 0.000224281 | 0 | 0 | −0.0033595 | −0.00187896 | −0.00017935 | −0.00351244 |
| 17930 | Myom2 | 0.00057009 | 0 | 0 | 0.002401789 | 0.001863516 | 0.001048401 | 0.003427406 |
| 66124 | Josd2 | −0.00080166 | 0 | 0 | −0.00282181 | −0.00185417 | −0.00152245 | −0.00300194 |
| 16514 | Kenj11 | −0.00037412 | 0 | 0 | −0.00241792 | −0.00185293 | 0 | 0 |
| 67426 | Coq8a | −3.37E−05 | 0 | 0 | 0.002314494 | 0.001852651 | −0.00053646 | 0.001889499 |
| 16004 | Igf2r | 0.00018751 | 0 | 0 | 0.003350439 | 0.001850841 | −0.00011303 | 0.001684541 |
| 16582 | Klfc3 | 0.000774127 | 0 | 0 | 0.001783138 | 0.00185009 | 0.000127471 | 0.001460774 |
| 67608 | Narf | −0.00063751 | 0 | 0 | −0.002043 | −0.0018489 | −0.0006837 | −0.00165112 |
| 20167 | Rtn2 | 0.000857736 | 0 | 0 | 0.002717896 | 0.001846268 | 0.000847129 | 0.00350395 |
| 76884 | Cyfip2 | 0.000240464 | 0 | 0 | 0.002562735 | 0.001844691 | 0.00036383 | 0.002505236 |
| 19652 | Rbm3 | 0.001156271 | 0 | 0 | 0.002035734 | 0.001841693 | 0.000216136 | 0.001663901 |
| 67198 | Spats2l | −0.00135132 | 0 | 0 | −0.00299567 | −0.00184044 | −0.00180675 | −0.00291922 |
| 64075 | Smoc1 | 0.000859137 | 0 | 0 | 0.001486427 | 0.001835036 | 0.001183758 | 0.002593273 |
| 333789 | N4bp2 | −0.00121355 | 0 | 0 | −0.00302428 | −0.00183586 | −0.00113087 | −0.00204353 |
| 338403 | Cndp1 | 0.00144391 | 0 | 0 | 0.003076941 | 0.00183325 | 0.00105116 | 0.001067418 |
| 14314 | Fstl1 | −0.00065049 | 0 | 0 | −0.00276451 | −0.00183162 | −0.00154694 | −0.00157364 |
| 12142 | Prdm1 | 0.000938424 | 0 | 0 | 0.001357265 | 0.001830109 | 0.000246566 | 0.000338097 |
| 242274 | Lrrc7 | −0.00028107 | 0 | 0 | −0.00164828 | −0.0018255 | −0.00013073 | 0 |
| 16918 | Myd | 0.000991448 | 0 | 0 | −0.00136427 | −0.00182475 | 0.000406033 | −0.00309162 |
| 22174 | Tyro3 | −0.00108425 | 0 | 0 | −0.00328713 | −0.00182372 | −0.00057532 | −0.0008032 |
| 327814 | Ppfia2 | 6.64E−05 | 0 | 0 | −0.00176106 | −0.00181519 | 0 | 0 |
| 106389 | Eaf2 | −7.74E−05 | 0 | 0 | 0.002596407 | 0.001813788 | −4.61E−05 | 0.002313475 |
| 16728 | L1cam | −0.00021599 | 0 | 0 | 0.002481056 | 0.001809646 | −0.00068907 | 0.00181221 |
| 60411 | Cenpk | −0.00103642 | 0 | 0 | −0.0034549 | −0.00180145 | −0.00152368 | −0.00234645 |
| 319660 | Agmo | −0.00010513 | 0 | 0 | 0.003143736 | 0.001801213 | 1.88E−05 | 0.00265476 |
| 55987 | Cpxm2 | −0.00042054 | 0 | 0 | 0.001528238 | 0.001800004 | −0.00014322 | 0.001946365 |
| 69066 | 1810010H24Rik | 0.00167297 | 0 | 0 | 0.001841863 | 0.001797445 | 0.001718363 | 0.001836541 |
| 69656 | Pir | −0.00018365 | 0 | 0 | 0.001795298 | 0.001796909 | 0.000497627 | 0.002516351 |
| 50794 | Klf13 | −0.00054489 | 0 | 0 | −0.00226403 | −0.0017958 | −0.00018275 | −0.00116319 |
| 107503 | Atf5 | 0.000557164 | 0 | 0 | 0.002442106 | 0.001795046 | 0.000988047 | 0.002661894 |
| 13139 | Dgka | −0.00176641 | 0 | 0 | −0.00273637 | −0.00179393 | −0.00189914 | −0.00169095 |
| 329278 | Tnn | −0.00035866 | 0 | 0 | −0.00218743 | −0.00178778 | −0.00027423 | 0 |
| 18074 | Nid2 | −0.00149173 | 0 | 0 | −0.00066419 | −0.001787 | −0.00127334 | −0.00155246 |
| 20317 | Serpinf1 | −0.00029289 | 0 | 0 | −0.00251132 | −0.00178643 | −0.00037075 | −0.00243231 |
| 20135 | Rrm2 | −0.0015768 | 0 | 0 | −0.00320778 | −0.00178531 | −0.00120071 | −0.00190569 |
| 207592 | Tbc1d16 | −0.00072351 | 0 | 0 | −0.00225964 | −0.00178434 | −0.00025065 | −0.00230778 |
| 20390 | Sftpd | −0.00102834 | 0 | 0 | 0 | −0.00178427 | −0.00068135 | 0 |
| 435766 | Tnni3k | 0 | 0 | 0 | 0 | 0.001783687 | 0 | 0 |
| 17754 | Map1a | 0 | 0 | 0 | 0.001744805 | 0.001781276 | 0 | 0.000457019 |
| 102278 | Cpne7 | −0.0008043 | 0 | 0 | 0.002412463 | 0.001780248 | 0 | 0 |
| 71911 | Bdh1 | −0.0005151 | 0 | 0 | −0.00065178 | −0.00177974 | −0.00178767 | −0.00238167 |
| 83771 | Tas1r3 | −0.0004431 | 0 | 0 | −0.00214429 | −0.00177316 | −0.00059779 | 0 |
| 53415 | Htatip2 | 0.000771143 | 0 | 0 | 0.003044173 | 0.00177276 | 0.001363725 | 0.002050444 |
| 71665 | Fuca1 | 0.000785624 | 0 | 0 | 0.003331589 | 0.001770636 | 0.000467985 | 0.002065108 |
| 16408 | Itgal | −0.00071117 | 0 | 0 | −0.00123309 | 0.0017682 | −0.00058173 | 0.001014637 |
| 74519 | Cyp2j9 | 0.001059752 | 0 | 0 | 0.002268073 | 0.00176049 | 0.000526319 | 0.001954165 |
| 12834 | Col6a2 | −0.00050596 | 0 | 0 | −0.00130436 | −0.00175761 | −0.00061656 | −0.00190564 |
| 17988 | Ndrg1 | −0.00142803 | 0 | 0 | 0.000805228 | 0.001751293 | −0.00129956 | 0.002009717 |
| 17755 | Map1b | 0.000485741 | 0 | 0 | 0.001128113 | 0.001748764 | 0.000375496 | 0.00178863 |
| 245595 | Zfp711 | 0 | 0 | 0 | −0.00348297 | −0.00174785 | 6.67E−05 | 0 |
| 232087 | Mat2a | −0.00100701 | 0 | 0 | −0.0032379 | −0.00174768 | −0.00124757 | −0.00331631 |
| 17112 | Tm4sf1 | 0.000337197 | 0 | 0 | 0.00258524 | 0.001746897 | 0.000410271 | 0.002054576 |
| 16797 | Lat | 0.001478147 | 0 | 0 | 0.001694977 | 0.001746654 | 0.001325592 | 0.00128542 |
| 208869 | Dock3 | −0.00019357 | 0 | 0 | 0.001279226 | 0.001743478 | −1.89E−05 | 0.001484781 |
| 108153 | Adamts7 | −0.00066541 | 0 | 0 | −0.00179825 | −0.001737 | −0.00200581 | −0.00314036 |
| 17181 | Matn2 | 0.000796153 | 0 | 0 | −0.00203957 | −0.00173661 | 0.00015428 | −0.00197935 |
| 268880 | Xxylt1 | −0.00028243 | 0 | 0 | 0.002985575 | 0.00173549 | −0.00020894 | 0.00145479 |
| 93761 | Smarca1 | −0.00129271 | 0 | 0 | −0.00256497 | −0.0017315 | −0.00049758 | −0.00274337 |
| 12323 | Camk2b | 0.000753444 | 0 | 0 | 0.00346901 | 0.001728993 | 0.001073744 | 0.001766788 |
| 432628 | Mfsd2b | 0.000488089 | 0 | 0 | 0.002457454 | 0.001727177 | −0.00033928 | 0.001570907 |
| 75906 | Fam184a | 0.001220169 | 0 | 0 | 0.003481714 | 0.001727151 | 0.000587632 | 0.00226467 |
| 17150 | Mfap2 | 2.79E−05 | 0 | 0 | −0.00133678 | −0.00172676 | 0.000101876 | −0.00191 |
| 268595 | D430019H16Rik | −0.00060574 | 0 | 0 | −0.0025612 | −0.00172547 | −0.00060154 | 0 |
| 66107 | Widc21 | 0.000134984 | 0 | 0 | −0.00105783 | 0.000188387 | 0.005170963 | 0 |
| 338365 | Slc41a2 | 0.001756835 | 0 | 0 | 0.001401054 | 0.001414444 | 0.003852465 | 0.003428308 |
| 22637 | Zap7O | 0.001906675 | 0 | 0 | −0.00043757 | 0.000265974 | 0.003749415 | 0.001959133 |
| 114564 | Csprs | 0 | 0 | 0 | 0 | 0 | 0.003732392 | 0 |
| 13048 | Cux2 | 0.000855938 | 0 | 0 | −0.00224083 | 0.00071916 | 0.003713718 | 0.001393093 |
| 192201 | Wfdc15b | 0.001699528 | 0 | 0 | 0 | 0 | 0.003573589 | 0 |
| 11459 | Acta1 | −0.00184139 | 0 | 0 | −0.00240103 | −0.00141005 | −0.00345988 | −0.00253871 |
| 19175 | Psmb6 | −0.00093177 | 0 | 0 | −0.0008807 | 9.66E−06 | −0.00327604 | −0.00193709 |
| 243881 | Cyp2b23 | 0 | 0 | 0 | 0 | 0 | 0.003266991 | 0 |
| 13077 | Cyp1a2 | −0.00163318 | 0 | 0 | 1.42E−05 | −0.00067467 | −0.00320041 | 0 |
| 108112 | Eif4ebp3 | −0.00151827 | 0 | 0 | −0.00053684 | −0.00022821 | −0.00315773 | −0.00155108 |
| 18606 | Enpp2 | −0.00179581 | 0 | 0 | −0.00240274 | −0.00135394 | −0.00315757 | −0.00254117 |
| 13706 | Cela2a | −0.00152112 | 0 | 0 | 0 | −0.00054905 | −0.00303513 | 0 |
| 56620 | Clec4n | 0.001361772 | 0 | 0 | −0.0022361 | 0.000836882 | 0.003018418 | 0.002790674 |
| 277753 | Cyp4a12a | 0.001257993 | 0 | 0 | 0 | 0 | 0.003010406 | 0 |
| 66786 | Olfr701 | 0.001607358 | 0 | 0 | 0 | 0 | 0.002939062 | 0 |
| 74147 | Ehhadh | −0.00181849 | 0 | 0 | −0.001466 | −0.00015616 | −0.00293245 | −0.00293517 |
| 667278 | Mup-ps16 | 0 | 0 | 0 | 0 | 0 | −0.00291165 | 0 |
| 20714 | Serplna3k | −0.00141171 | 0 | 0 | 0.001380092 | −5.49E−05 | 0.002883364 | 0 |
| 57349 | Ppbp | 0.001282298 | 0 | 0 | 0.001867503 | 0.001586206 | 0.002874124 | 0.00351731 |
| 25908 | Eif2s3y | 0.001853542 | 0 | 0 | 0.00045341 | 0.000610556 | 0.002867317 | 0.002089403 |
| 107141 | Cyp2c50 | −0.00186025 | 0 | 0 | 0 | 0 | −0.00269634 | 0 |
| 16891 | Lipg | −0.00192269 | 0 | 0 | −0.00181802 | −0.0014958 | −0.00265026 | −0.00332368 |
| 12346 | Car1 | −0.00112048 | 0 | 0 | −0.00078364 | −0.00097704 | −0.00261547 | 0 |
| 13982 | Esr1 | 0.001418545 | 0 | 0 | 0.001018317 | 0.001679815 | 0.002603477 | 0.001184146 |
| 14456 | Gas6 | 0.001734974 | 0 | 0 | −0.0013121 | 0.001133349 | 0.002602254 | 0.001902974 |
| 192200 | Wfdc12 | 0.000814999 | 0 | 0 | 0 | 0 | 0.002582855 | 0 |
| 108995 | Tbc1d10c | 0.001651305 | 0 | 0 | 0.000975448 | 0.000795933 | 0.002571238 | 0.000427752 |
| 13089 | Cyp2b13 | 0.001164354 | 0 | 0 | 0 | 0.000436417 | −0.00255855 | 0 |
| 53412 | Ppp1r3c | 0.001560178 | 0 | 0 | 0.000262299 | 0.001498005 | 0.002552347 | 0.002382627 |
| 18054 | Ngp | 0.001689236 | 0 | 0 | 0.002844925 | 0.000527801 | 0.002542303 | 0 |
| 58860 | Adamdec1 | 0.001418973 | 0 | 0 | −0.00126849 | 0.001376811 | 0.002518143 | −0.00024695 |
| 14468 | Gbp2b | −0.00146733 | 0 | 0 | 0 | −0.00026286 | −0.00251436 | 0 |
| 81489 | Dnajb1 | −0.00187676 | 0 | 0 | −0.00326991 | −0.00119127 | −0.00250972 | −0.00238064 |
| 13088 | Cyp2b1O | −0.00187061 | 0 | 0 | −0.00075082 | −0.00055708 | −0.00248067 | 0 |
| 14600 | Ghr | 0.001888622 | 0 | 0 | 0.001007909 | 0.001048772 | 0.002451761 | 0.001485015 |
| 18406 | Orm2 | 0.001263175 | 0 | 0 | 0 | 0 | 0.002449698 | 0 |
| 71754 | Cyp2d4O | −0.0012712 | 0 | 0 | 0 | 0 | −0.00243909 | 0 |
| 66175 | Mustn1 | 0.001739354 | 0 | 0 | 0.000301933 | 0.000955565 | 0.002430027 | 0.001251935 |
| 80890 | Trim2 | −0.00158691 | 0 | 0 | 0.000377848 | 0.000221197 | −0.00241149 | 0.000123936 |
| 20216 | Acsm3 | −0.00107974 | 0 | 0 | 0.002700532 | −2.74E−05 | −0.0024087 | −0.00125233 |
| 93842 | Igsf9 | −0.000881 | 0 | 0 | −0.00101484 | −0.00037881 | −0.00240688 | −0.00227923 |
| 17773 | Mtnr1a | 0.001762627 | 0 | 0 | 0 | 0 | 0.002394971 | 0 |
| 100689 | Spon2 | 0.0010646 | 0 | 0 | 0.00105781 | −2.70E−05 | 0.002392905 | 0.000620619 |
| 20720 | Serplne2 | 0.001160861 | 0 | 0 | 0.002170676 | 0.000674758 | 0.002372705 | 0.003279427 |
| 107995 | Cdc2O | −0.00180395 | 0 | 0 | −0.00208657 | −0.00101737 | −0.00235875 | −0.00132052 |
| 27047 | Omd | 0.000542991 | 0 | 0 | −0.00043146 | 0.000745999 | 0.002335787 | 0.00308452 |
| 433904 | Ociad2 | −0.00117157 | 0 | 0 | −0.00043997 | 0.000261449 | −0.00231236 | −0.00169879 |
| 50490 | Nox4 | −0.00103689 | 0 | 0 | −0.00033603 | −0.00137453 | −0.00230529 | −0.00045097 |
| 74488 | Lrrc15 | 0.001731788 | 0 | 0 | 0.002604405 | 0.001227238 | 0.00229405 | 0 |
| 20305 | Ccl6 | 0.00153504 | 0 | 0 | 0.002883577 | 0.000877377 | 0.002280313 | 0.001945755 |
| 195733 | Grhl1 | −0.00105935 | 0 | 0 | 0.000517114 | −1.06E−05 | −0.00226453 | −0.000971 |
| 100559 | Ugt2b3B | −0.00184073 | 0 | 0 | 0 | 0 | −0.00224743 | 0 |
| 213056 | Fam126b | −0.00096017 | 0 | 0 | −0.00080131 | −0.00039672 | −0.00222889 | −0.00116235 |
| 14262 | Fmo3 | −0.00089197 | 0 | 0 | −0.00086054 | 0.000433247 | −0.00222406 | −0.00013957 |
| 69099 | 1810009N02Rik | −0.00174195 | 0 | 0 | 0 | 0 | −0.0022183 | 0 |
| 12389 | Cav1 | −0.0013644 | 0 | 0 | −0.00162476 | −0.00082236 | −0.00221512 | −0.00210884 |
| 319278 | Shfl | 0.001429675 | 0 | 0 | 0.000817796 | 0.001232755 | 0.002203027 | 0.001318725 |
| 11858 | Rnd2 | 0.001746221 | 0 | 0 | 0.002679872 | 0.000811645 | 0.002193748 | 0.003194834 |
| 13992 | Khdrbs3 | −0.00161146 | 0 | 0 | −0.0016579 | −0.00086888 | −0.00218344 | −0.0012416 |
| 13214 | Defb1 | −0.00043799 | 0 | 0 | 0 | 0.001234223 | −0.00218009 | 0 |
| 18793 | Plaur | 0.000991205 | 0 | 0 | 0.002669278 | 0.000532865 | 0.002173119 | 0.002153533 |
| 53877 | Ear4 | 0 | 0 | 0 | 0 | 0 | 0.002171678 | 0 |
| 17864 | Mybl1 | 0.001010566 | 0 | 0 | −0.00139668 | 0.001365323 | 0.002162576 | 0 |
| 22784 | Slc30a3 | 0.001725316 | 0 | 0 | 0 | 0.001078989 | 0.002135723 | 0 |
| 269589 | Sytl1 | 0.001741266 | 0 | 0 | 0.000115906 | 0.000682026 | 0.002135431 | 0.000180554 |
| 633640 | Tmem267 | −0.00056525 | 0 | 0 | −6.22E−05 | −0.00043831 | −0.00212685 | −0.00335881 |
| 15486 | Hsd17b2 | −0.00164396 | 0 | 0 | −0.00166551 | −0.00041862 | −0.00212582 | 0 |
| 17836 | Mug1 | −0.00158149 | 0 | 0 | −0.00189719 | −0.00074472 | −0.00212542 | −0.00316537 |
| 238257 | Tmem30b | 0.001516711 | 0 | 0 | −0.00018088 | 0.000824416 | 0.002124708 | 0.000949149 |
| 67896 | Ccdc80 | −0.00152123 | 0 | 0 | −0.00200781 | −0.00091609 | −0.00212164 | −0.00195614 |
| 66442 | Spc25 | −0.00179427 | 0 | 0 | −0.00299932 | −0.00124897 | −0.00211909 | −0.00260427 |
| 17691 | Slk1 | 0.001565241 | 0 | 0 | 0.001295329 | 0.000852089 | 0.002114301 | 0.001195601 |
| 22249 | Unc13b | 0.000688648 | 0 | 0 | −0.0010031 | 0.000372494 | 0.002110785 | 0.002004303 |
| 64296 | Abhd8 | −0.00167768 | 0 | 0 | −0.00212983 | −0.00108841 | −0.00209492 | −0.00139819 |
| 63873 | Trpv4 | −0.0016122 | 0 | 0 | −0.0001883 | 0.001475848 | −0.00209432 | 0.000454598 |
| 102022 | Ces2a | −0.00132116 | 0 | 0 | −0.0011736 | 2.31E−05 | −0.00208936 | 0 |
| 231238 | Sel1l3 | −0.0015214 | 0 | 0 | −0.0007145 | −0.00060017 | −0.00208301 | −0.00059999 |
| 20848 | Stat3 | 0.001785549 | 0 | 0 | 0.002497216 | 0.001612677 | 0.002081173 | 0.002136357 |
| 13034 | Ctse | 0.000692329 | 0 | 0 | 0.001625428 | 0.001381126 | 0.00207847 | 0.002476103 |
| 100504404 | H2-Ea | 0 | 0 | 0 | 0 | 0 | −0.0020716 | 0 |
| 223453 | Dap | 0.000857168 | 0 | 0 | 0.001183326 | 0.000768795 | 0.002069118 | 0.001608181 |
| 72042 | Cotl1 | 0.000421258 | 0 | 0 | −0.00086449 | 0.000291571 | 0.002068474 | 0.001661327 |
| 71974 | Prmt3 | 0.001163606 | 0 | 0 | 0.001487062 | 0.000713935 | 0.002062023 | 0.001411312 |
| 229759 | Olfm3 | 0.001566466 | 0 | 0 | 0.001090067 | 0.000623322 | 0.002057012 | 0.000548063 |
| 23985 | Slc26a4 | 0.001755864 | 0 | 0 | 0.001917327 | 0.000337376 | 0.002053467 | 0.001597226 |
| 107449 | Unc5b | 0.000778694 | 0 | 0 | −0.00179841 | 0.00012058 | 0.002048688 | 0.000405657 |
| 71833 | Dcaf7 | 0.001475915 | 0 | 0 | 0.001036229 | 0.000628877 | 0.002046914 | 0.000903447 |
| 30937 | Lmcd1 | 0.001275556 | 0 | 0 | 0.001119174 | 0.00080501 | 0.002045698 | 0.001749398 |
| 66589 | Ube2v1 | 0.001076405 | 0 | 0 | −0.00011051 | 0.000850832 | 0.002044931 | 0.003091985 |
| 76886 | Fam81a | −0.00155983 | 0 | 0 | −0.00214643 | −9.58E−05 | −0.00204042 | −0.00060508 |
| 18778 | Pla2g1b | −0.00181502 | 0 | 0 | 0 | −0.00161645 | −0.00203206 | 0 |
| 14537 | Gcnt1 | −0.00144932 | 0 | 0 | −1.00E−05 | 0.000303696 | −0.00202274 | 0.000101614 |
| 434674 | Slc22a28 | 0 | 0 | 0 | 0 | 0 | 0.002010741 | 0 |
| 21828 | Thbs4 | 0.001463558 | 0 | 0 | 0.001511105 | 0.001489933 | 0.002008552 | 0.002416744 |
| 66607 | Ms4a4d | 0.001854538 | 0 | 0 | 0.003378096 | 0.001000081 | 0.001986549 | 0 |
| 94190 | Ophn1 | −0.00187027 | 0 | 0 | −0.0009281 | −0.00027958 | −0.00198549 | −0.00045441 |
| 12721 | Coro1a | 0.000894552 | 0 | 0 | −0.00248535 | 0.000216472 | 0.001983989 | 0.000346115 |
| 230162 | Zfp189 | −0.00186977 | 0 | 0 | −0.00014542 | −0.00088152 | −0.00197988 | −0.00162805 |
| 12722 | Clca3a1 | 0.001866925 | 0 | 0 | 0.000688151 | 0.000711377 | 0.001976557 | 0 |
| 12370 | Casp8 | −0.00114968 | 0 | 0 | −0.00232962 | −0.00114189 | −0.00197538 | −0.00195458 |
| 19733 | Rgn | −0.00131334 | 0 | 0 | 0.002624706 | −0.0002543 | −0.0019723 | 0 |
| 21916 | Tmod1 | 0.001700512 | 0 | 0 | −0.00032051 | 0.000607263 | 0.001968498 | −0.00079585 |
| 12297 | Cacnb3 | −0.00071385 | 0 | 0 | −0.00049038 | −0.0003588 | −0.00196477 | −0.00031662 |
| 170639 | Olfr78 | −0.00191278 | 0 | 0 | −0.00106077 | −0.00148827 | −0.00196142 | 0 |
| 57294 | Rps27 | −0.00169263 | 0 | 0 | −0.00247711 | −0.00056627 | −0.00195233 | −0.00195208 |
| 71907 | Serpina9 | 0.001010124 | 0 | 0 | 0 | 0 | 0.001943053 | 0 |
| 20725 | Serpinb8 | 0.001610705 | 0 | 0 | −0.00177975 | 0.000332236 | 0.001940237 | −0.00046095 |
| 71684 | Rbm43 | 0.001558662 | 0 | 0 | 0.000904586 | 0.000179452 | 0.001937747 | 0.000183022 |
| 16612 | Klk1 | −0.0011783 | 0 | 0 | 0 | 0 | −0.00193439 | 0 |
| 233005 | Cyp2a22 | 0.00082722 | 0 | 0 | 0 | 0 | 0.001930079 | 0 |
| 67867 | Lrrc28 | −0.00138779 | 0 | 0 | 0.001130932 | 0.000643237 | −0.00192602 | 0.000290407 |
| 11655 | Alas1 | −0.00124342 | 0 | 0 | 0.000256605 | −0.00124776 | −0.00192343 | −0.00217486 |
| 50850 | Spast | −0.00150001 | 0 | 0 | −0.00068239 | −0.00074909 | −0.00191818 | −0.00137467 |
| 11857 | Arhgdlb | 0.001643664 | 0 | 0 | 0.002179589 | 0.001000387 | 0.001914208 | 0.001014974 |
| 17313 | Mgp | −0.00114033 | 0 | 0 | 0.001877691 | 0.000362013 | −0.00191311 | 0.000142574 |
| 26451 | Rpl27a | −0.00129667 | 0 | 0 | −0.00138057 | −0.00095569 | −0.00191042 | −0.0017168 |
| 17394 | Mmp8 | 0.001609646 | 0 | 0 | 0.000941533 | 0.0009288 | 0.001909487 | 0 |
| 18639 | Pfkfb1 | −0.00116165 | 0 | 0 | −0.00107457 | −0.00101619 | −0.00190906 | −0.00200398 |
| 319710 | Frmd6 | −0.00189998 | 0 | 0 | −0.00044384 | 0.000327891 | −0.00190848 | −0.00046028 |
| 69165 | Cd209b | 0.001178984 | 0 | 0 | −0.00087307 | −5.17E−05 | 0.001906905 | 0.001167739 |
| 13909 | Ces3b | −0.00093291 | 0 | 0 | 0 | 0 | −0.00190463 | 0 |
| 209186 | Acnat2 | 0.001394725 | 0 | 0 | 0 | 0.00075792 | 0.001901116 | 0 |
| 20529 | Slc31a1 | −0.00071804 | 0 | 0 | −0.0014587 | −0.00152215 | −0.00190045 | −0.0024368 |
| 13190 | Dct | −0.00059207 | 0 | 0 | 0.000478812 | 0.000177777 | 0.001899867 | 0.002976067 |
| 108148 | Galnt2 | 0.001031003 | 0 | 0 | −0.00050914 | 0.000169462 | 0.001897845 | 0.000307808 |
| 19683 | Rdh16 | 0.001457673 | 0 | 0 | 0.000508686 | 0.000125652 | 0.001897236 | 0 |
| 15902 | Id2 | 0.001454249 | 0 | 0 | 0.001288871 | 0.000414655 | 0.001896984 | 0.001035394 |
| 622473 | Ripply1 | −0.00067387 | 0 | 0 | 0 | 0 | −0.0018961 | 0 |
| 50702 | Cfhr1 | 0.001569172 | 0 | 0 | 0 | 1.17E−05 | 0.001894463 | 0 |
| 223921 | Aaas | −0.00152203 | 0 | 0 | 0.002868986 | 8.13E−06 | −0.00189336 | 0.000771327 |
| 70683 | Utp2O | 0.001519927 | 0 | 0 | 7.22E−05 | 0.000384633 | 0.001893166 | 0.000586983 |
| 211666 | Mgst2 | −0.00060962 | 0 | 0 | −0.00143753 | −0.00027687 | −0.0018927 | 0 |
| 237459 | Cdk17 | 0.000992665 | 0 | 0 | 0.000632144 | 0.000102417 | 0.001891028 | 0.00092605 |
| 14420 | Galc | 0.001203736 | 0 | 0 | 0.002482384 | 0.001517231 | 0.001887882 | 0.00213373 |
| 235505 | Cd109 | −0.00178839 | 0 | 0 | −0.0002289 | −0.0001508 | −0.00188678 | 0.00133606 |
| 54418 | Fmn2 | 7.81E−05 | 0 | 0 | 0.001876888 | 0.000571696 | 0.001883983 | 0.002999275 |
| 15270 | H2ax | −0.00139293 | 0 | 0 | 2.26E−05 | −0.00036711 | −0.00188181 | −0.00124313 |
| 94175 | Hrg | −0.00173916 | 0 | 0 | −0.0016305 | −0.00014764 | −0.00188019 | 0 |
| 14972 | H2-K1 | 0.000479516 | 0 | 0 | 0.002514759 | 0.000668064 | 0.00187988 | 0.00199963 |
| 207920 | Esrp1 | 0.001447467 | 0 | 0 | 0.001187429 | 0.000486242 | 0.001878774 | 0 |
| 11566 | Adss | 0.001892475 | 0 | 0 | 0.002053039 | 0.001479284 | 0.001875719 | 0.002000998 |
| 13360 | Dhcr7 | 0.001868037 | 0 | 0 | 0.000825809 | 0.000105151 | 0.0018741 | −0.00026254 |
| 68067 | Mrnip | 0.001862177 | 0 | 0 | 0.002149218 | 0.000551116 | 0.001872926 | 0.00039619 |
| 13074 | Cyp17a1 | −0.00172116 | 0 | 0 | 0.000279523 | 0.000199278 | −0.00187015 | 0.000700253 |
| 77318 | Ankrd55 | 0.000998024 | 0 | 0 | 0.001422258 | 0.001113108 | 0.001868651 | 0 |
| 258216 | Olfr1034 | 0 | 0 | 0 | 0 | 0 | −0.00186851 | 0 |
| 268756 | Gulo | −0.00149556 | 0 | 0 | 0 | −0.0009474 | −0.00186703 | 0 |
| 16186 | Il2rg | 0.001907225 | 0 | 0 | −0.00134739 | 0.000652972 | 0.001864169 | −0.00024208 |
| 71729 | Rgs12 | 0.000859626 | 0 | 0 | 0.001672631 | 0.000595427 | 0.001851488 | 0.000997979 |
| 234673 | Ces2e | −0.00029825 | 0 | 0 | 0.001775544 | 0.000771833 | −0.00184715 | −0.00014952 |
| 76971 | Sult2a8 | −0.00137313 | 0 | 0 | 0 | −0.00115793 | −0.00184677 | 0 |
| 20660 | Sorl1 | 5.57E−05 | 0 | 0 | −0.00161859 | −0.00062641 | 0.001845976 | 0.000430075 |
| 56739 | Rec8 | −0.00177594 | 0 | 0 | −0.00109433 | −0.00024239 | −0.00184378 | −0.00023613 |
| 20867 | Stip1 | −0.00082337 | 0 | 0 | −0.00083296 | −0.00071304 | −0.00184224 | −0.00171122 |
| 217258 | Abca8a | −0.00139376 | 0 | 0 | −0.0005796 | 0.000108978 | −0.00184085 | −0.00148558 |
| 216350 | Tspan8 | 0.001294437 | 0 | 0 | −0.00021663 | 0.000111957 | 0.001837086 | −0.00146813 |
| 12162 | Bmp7 | 0.001317246 | 0 | 0 | −0.00159046 | 0.000797645 | 0.001834613 | −0.00022305 |
| 12140 | Fabp7 | −0.00160677 | 0 | 0 | −0.00191814 | −0.00106782 | −0.00183223 | 0 |
| 319170 | H2ac22 | 0 | 0 | 0 | 0 | 0 | −0.0018319 | 0 |
| 380969 | Nckap5l | −0.00149662 | 0 | 0 | −0.0028153 | −0.00105743 | −0.00183118 | −0.00261645 |
| 74264 | Rnf138rt1 | 0.001052205 | 0 | 0 | 0 | 0 | 0.001830305 | 0 |
| 19361 | Rad51 | −0.00160343 | 0 | 0 | −0.0017088 | −0.00111698 | −0.00182935 | 0 |
| 381530 | Mup20 | −0.00043516 | 0 | 0 | 0 | 0 | 0.001827134 | 0 |
| 74103 | Nebl | 0.000947411 | 0 | 0 | −0.00066663 | 0.00038502 | 0.00182376 | 0.0013426 |
| 109624 | Cald1 | 0.001403867 | 0 | 0 | 0.001363121 | 0.000862685 | 0.001822721 | 0.001439235 |
| 76415 | Fam187b | −0.00146467 | 0 | 0 | −0.00085096 | −0.0003184 | −0.00181857 | −0.00069418 |
| 12298 | Cacnb4 | 0.001308131 | 0 | 0 | 0.000303147 | 0.001595058 | 0.001818384 | 0.001923517 |
| 78070 | Cpt1c | −0.00093398 | 0 | 0 | −0.00075077 | 1.07E−05 | −0.00181624 | −0.00097885 |
| 53625 | B3gnt2 | 0.00108382 | 0 | 0 | 0.002769458 | 0.000692395 | 0.001814467 | 0.001659575 |
| 18171 | Nr1i2 | −0.00165632 | 0 | 0 | −0.00137957 | −0.00052063 | −0.00181413 | −0.000562 |
| 13124 | Cyp8b1 | −0.00106461 | 0 | 0 | −0.00300066 | 0.000195276 | −0.00181317 | −0.00074627 |
| 233870 | Tufm | −0.00162942 | 0 | 0 | −0.00115462 | −0.00095444 | −0.00181164 | −0.0021349 |
| 12768 | Ccr1 | 0.001828511 | 0 | 0 | 0.003228606 | 0.001177659 | 0.001810652 | 0.002342314 |
| 74868 | Tmem65 | 0.001315123 | 0 | 0 | 0.001285425 | 0.000546296 | 0.001809017 | 0.001255404 |
| 16420 | Itgb6 | 0.000973532 | 0 | 0 | −0.00121977 | −0.0001573 | 0.001804541 | −0.00023413 |
| 66447 | Mgst3 | 0.000570004 | 0 | 0 | 0.001600003 | 0.000634536 | 0.00180437 | 0.001986006 |
| 22024 | Crisp2 | 0.001908358 | 0 | 0 | 0.002987708 | 0.000919555 | 0.001802794 | 0 |
| 74145 | F13a1 | 0.000550621 | 0 | 0 | 0.002982141 | 0.001709769 | 0.001767174 | 0.00473652 |
| 96875 | Prg4 | 0.000378419 | 0 | 0 | 0.00155383 | 0.001231376 | 0.001306283 | 0.004158892 |
| 69585 | Hjv | −0.00165934 | 0 | 0 | −0.0029857 | −0.00074767 | −0.00092045 | −0.00365872 |
| 13506 | Dsc2 | 0.00097316 | 0 | 0 | −0.00337255 | −0.00082822 | 0.000850014 | −0.00347199 |
| 12035 | Bcat1 | 0.00019593 | 0 | 0 | −0.0023234 | −0.00070483 | 0.00015956 | −0.00338467 |
| 14778 | Gpx3 | 0.001659892 | 0 | 0 | 0.001951293 | 0.00142019 | 0.001594166 | 0.003359281 |
| 105859 | Csdc2 | −0.00069301 | 0 | 0 | −0.00216224 | −0.00169411 | −0.00168338 | −0.00332587 |
| 76905 | Lrg1 | 0.001582906 | 0 | 0 | 0.002886439 | 0.001533992 | 0.00119503 | 0.003303644 |
| 26364 | Adgre5 | 0.000501004 | 0 | 0 | 0.002878169 | 0.001343505 | 0.001442143 | 0.003288411 |
| 59095 | Fxyd6 | −0.00040692 | 0 | 0 | 0.002393025 | 0.001152893 | −0.00044838 | 0.003239958 |
| 11517 | Adcyap1r1 | 0.000441618 | 0 | 0 | 0.001683055 | 0.001671282 | 8.04E−05 | 0.003209802 |
| 52882 | Rgs7bp | 0 | 0 | 0 | −0.00245646 | −0.00155873 | 0 | −0.00320851 |
| 22354 | Vipr1 | 0.00089969 | 0 | 0 | −0.00028796 | −0.00102803 | −0.00012837 | −0.00318253 |
| 14718 | Got1 | −0.00039812 | 0 | 0 | 0.002352973 | 0.001114772 | −0.00058687 | 0.003180168 |
| 22142 | Tubala | −0.00090704 | 0 | 0 | −0.00225815 | −0.00155432 | −0.00172193 | −0.00317012 |
| 69816 | Mzb1 | −0.00127477 | 0 | 0 | 0.002635361 | 0.001706096 | 0.000621702 | 0.00314841 |
| 19417 | Rasgrfl | −0.00076243 | 0 | 0 | 0.003214021 | 0.0014042 | −0.00074631 | 0.003101063 |
| 78925 | Srd5a1 | −0.00046364 | 0 | 0 | −4.68E−05 | −0.00010638 | −0.00160332 | −0.00307069 |
| 70350 | Basp1 | −0.00012482 | 0 | 0 | −0.00323112 | −0.00160095 | −0.00066822 | −0.00298535 |
| 192190 | Pkhd1l1 | −0.0008985 | 0 | 0 | −0.00286295 | −0.00126599 | −0.00151642 | −0.00298485 |
| 17294 | Mest | 0.000349573 | 0 | 0 | −0.0004134 | −0.00108209 | 0.000311979 | −0.00296877 |
| 69675 | Pxdn | −0.00035498 | 0 | 0 | −0.00170771 | −0.00130243 | −0.00057858 | −0.00292545 |
| 140491 | Ppp1r3a | 0.001340148 | 0 | 0 | 0.001724518 | 0.000475076 | 0.001574309 | 0.002907198 |
| 246728 | Oas2 | 0.000113777 | 0 | 0 | 0.002254213 | 0.001181368 | 0.000203795 | 0.00285196 |
| 13051 | Cx3cr1 | 0.001438584 | 0 | 0 | 0.002904208 | 0.001245567 | 0.001628016 | 0.002849908 |
| 434130 | Ccdc8 | −0.00183421 | 0 | 0 | −0.00306221 | −0.00143709 | −0.0010411 | −0.0028373 |
| 226049 | Dmrt2 | 0.000433624 | 0 | 0 | 0.002924044 | 0.001311024 | 0.000208236 | 0.002820996 |
| 269116 | Nfasc | 0.000213099 | 0 | 0 | 0.001996544 | 0.001594537 | −7.99E−05 | 0.002812314 |
| 66333 | Aqp11 | −0.00030259 | 0 | 0 | −0.00247786 | −0.0009454 | −0.00085732 | −0.00281011 |
| 14051 | Eya4 | 0 | 0 | 0 | 0.002899838 | 0.001648661 | 0 | 0.002779846 |
| 107272 | Psat1 | −0.00092451 | 0 | 0 | 0.003382926 | 0.000956238 | 0.000217389 | 0.00277972 |
| 103149 | Upb1 | 0.00024728 | 0 | 0 | 0.000768075 | 9.38E−05 | −0.00029189 | −0.00276205 |
| 52187 | Rragd | 0.000367587 | 0 | 0 | 0.002212059 | 0.001392912 | 0.000553076 | 0.002761089 |
| 18227 | Nr4a2 | 0.000646749 | 0 | 0 | 0.002578609 | 0.001724993 | 0.000885791 | 0.002754854 |
| 27425 | Atp5l | −0.000144 | 0 | 0 | −0.00040837 | −0.0004315 | −0.00142889 | −0.00275092 |
| 12443 | Ccnd1 | 0.001253145 | 0 | 0 | −0.00048228 | −0.00070937 | −0.00116241 | −0.00274456 |
| 78894 | Aacs | −0.00031457 | 0 | 0 | −0.0019783 | −0.00111347 | −0.00033763 | −0.00273612 |
| 66197 | Cks2 | −0.00111812 | 0 | 0 | −0.00345045 | −0.00138053 | −0.00132017 | −0.00273105 |
| 14114 | Fbln1 | 0.001429377 | 0 | 0 | −0.00164756 | −0.00109297 | 0.000502165 | 0.00272677 |
| 76041 | Ccdc125 | −0.00142208 | 0 | 0 | −0.00311591 | −0.00103705 | −0.00136955 | −0.00272248 |
| 21366 | Slc6a6 | 0.000253526 | 0 | 0 | 0.003077293 | 0.001495857 | −0.0002804 | 0.002722247 |
| 71934 | Car13 | 0 | 0 | 0 | 0.002412124 | 0.001591526 | 0.000618754 | 0.002716558 |
| 20365 | Serf1 | 0.000214386 | 0 | 0 | −0.00215979 | −0.00171109 | −0.00017556 | −0.00271155 |
| 219132 | Phf11d | −7.70E−05 | 0 | 0 | 0.003028827 | 0.001045408 | 0.000763834 | 0.002706427 |
| 20541 | Slc8a1 | −0.00094992 | 0 | 0 | −0.00143581 | −0.00110075 | −0.00134384 | −0.00270147 |
| 17388 | Mmp15 | 0.000264013 | 0 | 0 | −0.00204634 | −0.00098789 | −0.00056028 | −0.00269315 |
| 13131 | Dab1 | 0.001440461 | 0 | 0 | 0.002667988 | 0.001502465 | 0.001646492 | 0.002688296 |
| 54409 | Ramp2 | 0.000481038 | 0 | 0 | −0.00315856 | −0.00171383 | 3.94E−06 | −0.00268803 |
| 60533 | Cd274 | −0.00186439 | 0 | 0 | −0.00175294 | 0.000939767 | −0.00107135 | 0.002679458 |
| 56312 | Nupr1 | 0.001183592 | 0 | 0 | 0.002924353 | 0.001045635 | 0.001353455 | 0.0026787 |
| 53896 | Slc7a10 | 0.000140002 | 0 | 0 | −0.002037 | −0.00153827 | −0.00061255 | −0.00267425 |
| 16777 | Lamb1 | 0.000152121 | 0 | 0 | −0.00277096 | −0.00140497 | −0.00010069 | −0.00266026 |
| 268930 | Pkmyt1 | −0.00011573 | 0 | 0 | −0.00118561 | −0.00093438 | −0.00120549 | −0.00264762 |
| 71664 | Mettl7b | 0.000229539 | 0 | 0 | −0.00207707 | −0.00073399 | 0.000576796 | −0.00264693 |
| 12501 | Cd3e | 0.001272056 | 0 | 0 | 0.002637364 | 0.001355632 | 0.00070099 | 0.00263794 |
| 77015 | Mpped2 | −0.00085289 | 0 | 0 | −0.00217162 | −0.00087096 | −0.00041545 | −0.00262857 |
| 407800 | Ecm2 | 0.000232366 | 0 | 0 | −0.00341577 | −0.00163036 | −0.00012596 | −0.00262423 |
| 239273 | Abcc4 | 0.000274337 | 0 | 0 | 0.000132127 | 0.000861564 | 0.001112822 | 0.002618813 |
| 54486 | Hpgds | 0.001278125 | 0 | 0 | 0.003399185 | 0.001660234 | 0.001091515 | 0.002616548 |
| 13180 | Pcbd1 | 0.000685504 | 0 | 0 | 0.001856368 | 0.001634066 | 0.001239347 | 0.002614419 |
| 14544 | Gda | −0.0008066 | 0 | 0 | 0.001877065 | 0.001454262 | −0.00050353 | 0.002610747 |
| 78751 | Zc3h6 | 0.000596382 | 0 | 0 | 0.002640954 | 0.001568118 | 0.000924542 | 0.002594595 |
| 50909 | C1ra | 0.000786486 | 0 | 0 | 0.003077715 | 0.001532784 | 0.000649584 | 0.002586999 |
| 30951 | Chx8 | −4.09E−05 | 0 | 0 | −0.00272502 | −0.00156998 | −0.0003623 | −0.00258025 |
| 68075 | Lurap1 | −0.00066969 | 0 | 0 | 0.003114697 | 0.001616073 | 0 | 0.002569925 |
| 72713 | Angptl1 | −0.00015149 | 0 | 0 | −0.00268212 | −0.00138679 | −0.00044174 | −0.00256485 |
| 53601 | Pcdh12 | 0.001030657 | 0 | 0 | −0.0018943 | −0.00123657 | 0.00070281 | −0.00255833 |
| 14254 | Flt1 | 0.00079163 | 0 | 0 | 0.003409108 | 0.00106227 | 0.000503781 | 0.002555301 |
| 74470 | Cep72 | −0.00050286 | 0 | 0 | −0.00188421 | −0.00100171 | −0.00108816 | −0.00255362 |
| 69993 | Chn2 | −0.00072132 | 0 | 0 | −0.00265315 | −0.00163479 | −0.00092273 | −0.00254209 |
| 171531 | Mlph | 0.000361527 | 0 | 0 | 0.00179232 | 0.001501435 | 0.001314038 | 0.002541833 |
| 108114 | Slc22a7 | −0.00134479 | 0 | 0 | −0.00031971 | −0.00083815 | −0.00117488 | −0.00253468 |
| 12428 | Ccna2 | −0.00058673 | 0 | 0 | −0.0022596 | −0.00135931 | −0.00104937 | −0.00253002 |
| 67383 | Carnmt1 | 0.000111518 | 0 | 0 | −0.00113141 | −0.00043542 | −0.00136678 | −0.00251143 |
| 26559 | Hunk | −3.90E−05 | 0 | 0 | −0.00093957 | −0.00128362 | −0.00054513 | −0.0025028 |
| 448987 | Fbxl7 | −0.00013011 | 0 | 0 | −0.00237847 | −0.00161139 | 0 | −0.00248959 |
| 71819 | Klf23 | −0.00067151 | 0 | 0 | −0.00117695 | −0.00145003 | −0.00151069 | −0.00248006 |
| 60613 | Kcnq4 | −0.00021426 | 0 | 0 | 0.000344972 | −0.00022512 | −0.00122448 | −0.00247984 |
| 17389 | Mmp16 | −0.00017474 | 0 | 0 | −0.0020705 | −0.00141568 | −0.00031547 | −0.0024775 |
| 16409 | Itgam | −0.00065516 | 0 | 0 | 0.001213699 | 9.80E−05 | 0.000194522 | 0.002470417 |
| 14700 | Gng10 | −0.00036327 | 0 | 0 | −0.00279248 | −0.00124007 | −0.00068751 | −0.00246848 |
| 241694 | Ralgapa2 | −0.00123773 | 0 | 0 | 0.002932627 | 0.001161646 | −2.81E−05 | 0.002466898 |
| 20250 | Scd2 | 0.001532092 | 0 | 0 | −0.00063353 | −5.32E−05 | −0.00019119 | −0.00246494 |
| 50528 | Tmprss2 | 0.001093799 | 0 | 0 | 0.002772927 | 0.001191787 | 0.001298665 | 0.002462908 |
| 380921 | Dgkh | −0.00102321 | 0 | 0 | 0.000718294 | 1.55E−05 | −0.00136157 | −0.00246087 |
| 16874 | Lhx6 | −0.00112797 | 0 | 0 | −0.00243728 | −0.00147186 | −0.00049384 | −0.0024495 |
| 66929 | Asf1b | −0.00011915 | 0 | 0 | 0.00231218 | 0.000496804 | 0.001130647 | 0.002439522 |
| 15463 | Agfg1 | −0.00056159 | 0 | 0 | −0.00070312 | −0.0008526 | −0.00042756 | −0.00243556 |
| 277978 | Exoc3l | 0.000364463 | 0 | 0 | −0.00194252 | −0.00122949 | 3.55E−06 | −0.00242822 |
| 52829 | Lurap1l | −0.00037645 | 0 | 0 | −0.00142623 | −0.00160414 | 0.00028591 | −0.00242414 |
| 214951 | Rhbdl1 | 0.000175638 | 0 | 0 | −0.00160673 | −0.00133277 | −0.00048389 | −0.002419 |
| 16399 | Itga2b | −0.00023451 | 0 | 0 | 0.002425346 | 0.001551959 | −0.00034057 | 0.00241592 |
| 66094 | Lsm7 | −0.00044894 | 0 | 0 | −0.00190314 | −0.00089031 | −0.00079098 | −0.00241415 |
| 73173 | Pcdh18 | −0.00036266 | 0 | 0 | −0.00302625 | −0.00139083 | −0.00035757 | −0.00240527 |
| 229474 | Fhdc1 | 0.000793623 | 0 | 0 | 0.001156443 | 0.000866493 | 0.001191548 | 0.002402544 |
| 432530 | Adcy1 | 0.001129307 | 0 | 0 | 0.002797078 | 0.001664645 | 0.001002216 | 0.002379085 |
| 13076 | Cyp1a1 | −0.00078106 | 0 | 0 | −0.00277137 | −0.00149235 | −0.001092 | −0.00237687 |
| 67674 | Trmt112 | −0.00085519 | 0 | 0 | −0.00276182 | −0.00111953 | −0.00103389 | −0.0023764 |
| 11684 | Alox12 | −0.00119043 | 0 | 0 | 0.002102185 | 0.001484392 | −0.00040564 | 0.002360773 |
| 74393 | Map10 | −0.00019921 | 0 | 0 | −0.00191692 | −0.00127128 | −0.00035379 | −0.00235114 |
| 56532 | Ripk3 | −0.0007649 | 0 | 0 | −0.00271538 | −0.0010278 | −0.00037295 | −0.0023465 |
| 243910 | Nfkbid | −0.00075782 | 0 | 0 | −0.00235456 | −0.00095894 | −0.00167028 | −0.00234398 |
| 11807 | Apoa2 | 0.001516251 | 0 | 0 | −0.00082074 | −0.00029972 | 0.001350025 | −0.0023406 |
| 26381 | Esrrg | 0.000823429 | 0 | 0 | 0.002356569 | 0.000567121 | 0.001371054 | 0.002324844 |
| 80861 | Dhx58 | 0.001262507 | 0 | 0 | 0.003031954 | 0.001147669 | 0.001728522 | 0.00231764 |
| 269831 | Tspan12 | −0.0003825 | 0 | 0 | −0.00246523 | −0.00121115 | −0.00053015 | −0.00231746 |
| 319387 | Adgrl3 | −0.00040975 | 0 | 0 | −0.00091601 | −0.00122604 | −0.00084207 | −0.00231726 |
| 70445 | Cd248 | 0.000391857 | 0 | 0 | −0.00191459 | −0.00112943 | −0.00090711 | −0.00230933 |
| 12953 | Cry2 | 0.000460844 | 0 | 0 | 0.001515918 | 0.001406015 | 0.000684616 | 0.002305944 |
| 118449 | Synpo2 | −0.00177981 | 0 | 0 | −0.00028103 | 0.000904777 | −0.00076233 | 0.002305884 |
| 320844 | Amigo3 | 5.17E−05 | 0 | 0 | −0.00116322 | −0.00078388 | −0.00033083 | −0.00230367 |
| 330189 | Tmem120b | −0.00183389 | 0 | 0 | −0.00267857 | −0.00125361 | −0.00164893 | −0.00230262 |
| 18552 | Pcsk5 | 0.000127645 | 0 | 0 | −0.00171744 | −0.00132417 | 0.000196847 | −0.00228946 |
| 71653 | Shtn1 | 5.61E−06 | 0 | 0 | 0.002453786 | 0.001467721 | 0.00060886 | 0.002285828 |
| 69590 | Gpx8 | −6.12E−05 | 0 | 0 | −0.00268215 | −0.0016704 | −2.42E−05 | −0.00228397 |
| 21857 | Timp1 | 0.000974392 | 0 | 0 | 0.001655421 | 0.001084845 | 0.000895719 | 0.002283378 |
| 20517 | Slc22a1 | −0.0009961 | 0 | 0 | 0.002234931 | 0.000790866 | −0.00031884 | 0.002279592 |
| 14211 | Smc2 | −0.00090744 | 0 | 0 | −0.00197129 | −0.00060157 | −0.00135583 | −0.00227956 |
| 242341 | Atp6v0d2 | 0.000440131 | 0 | 0 | −0.0005878 | 0.001380763 | 0.000801626 | 0.00227558 |
| 12817 | Col13a1 | −0.00038997 | 0 | 0 | −0.00239904 | −0.00127665 | −0.00089483 | −0.00227456 |
| 20356 | Sema5a | −0.00107188 | 0 | 0 | −0.00224062 | −0.00144448 | −0.00084255 | −0.00227199 |
| 11568 | Aebp1 | 0.000767307 | 0 | 0 | 0.00041545 | 0.001172205 | 0.000960704 | 0.002271028 |
| 11496 | Adam22 | −0.00018151 | 0 | 0 | 0.003324146 | 0.001400661 | 0.00015571 | 0.002266282 |
| 24055 | Sh3bp2 | −0.00038987 | 0 | 0 | −0.00202873 | −0.00096446 | −0.0006756 | −0.00226552 |
| 140483 | Hnmt | −0.00020217 | 0 | 0 | 0.001287447 | 0.000584764 | 0.000387536 | 0.002262353 |
| 14799 | Gria1 | −0.00040777 | 0 | 0 | −0.00314039 | −0.00149107 | −0.00021196 | −0.00225485 |
| 268373 | Ppla | −0.00076777 | 0 | 0 | −0.00140064 | −0.00053116 | −0.00111992 | −0.00225394 |
| 66421 | 2410004B18Rik | −0.00059433 | 0 | 0 | −0.0022595 | −0.00135835 | −0.00067563 | −0.00225079 |
| 56711 | Plag1 | −0.00050386 | 0 | 0 | 0.001717009 | 0.000851856 | 0.00052785 | 0.002248219 |
| 73368 | Col20a1 | 0.00024395 | 0 | 0 | −0.00198544 | −0.0008666 | −0.00033934 | −0.00224691 |
| 244152 | Tsku | 0.000259503 | 0 | 0 | −0.00022098 | −0.00044548 | −0.00151328 | −0.00223897 |
| 64008 | Aqp9 | 0.001104498 | 0 | 0 | 0.001738072 | 0.000856077 | 0.000913388 | 0.002237822 |
| 21752 | Tert | 0.001427647 | 0 | 0 | −0.0011056 | −0.00052574 | 0.000139753 | −0.00223744 |
| 13837 | Epha3 | 0.000488457 | 0 | 0 | 3.77E−05 | −0.00030261 | −0.00064563 | −0.00223524 |
| 78388 | Mvp | 0.000576743 | 0 | 0 | 0.001939904 | 0.001717745 | 0.001007456 | 0.002224977 |
| 15016 | H2-Q5 | 0.000553676 | 0 | 0 | 0.002299578 | 0.001272567 | 0.001579111 | 0.00222414 |
| 104174 | Gldc | 0.000517859 | 0 | 0 | 0.003356855 | 0.001590793 | 0.00037782 | 0.002224031 |
| 13557 | E2f3 | −0.00118412 | 0 | 0 | −0.00154231 | −0.00085701 | −0.00121164 | −0.00222226 |
| 20090 | Rps29 | −0.00093262 | 0 | 0 | −0.0015731 | −0.0005854 | −0.00166991 | −0.00222208 |
| 319504 | Nrcam | 0.000639569 | 0 | 0 | −0.00227502 | −0.00105328 | 0.000975268 | −0.00222092 |
| 399558 | Flrt2 | 0.000616885 | 0 | 0 | 0.001362183 | 0.000369401 | −0.0010932 | −0.00221931 |
| 241556 | Tspan18 | 1.34E−05 | 0 | 0 | −0.00161817 | −0.00088225 | 4.26E−05 | −0.00221914 |
| 246730 | Oas1a | 8.72E−05 | 0 | 0 | 0.001328752 | 0.000938177 | 4.79E−05 | 0.002214851 |
| 19013 | Ppara | 0.000383539 | 0 | 0 | 0.002837071 | 0.00125887 | −0.00054745 | 0.002208901 |
| 74318 | Hopx | 0.001057224 | 0 | 0 | −0.00249587 | −0.00090658 | 0.00086841 | −0.00220879 |
| 268890 | Lsamp | −0.00077802 | 0 | 0 | −0.00094975 | −0.00106048 | 0 | −0.00220225 |
| 106861 | Abhd3 | −0.00036406 | 0 | 0 | 0.002608364 | 0.000823279 | 0.00040195 | 0.002195931 |
| 22271 | Upp1 | −0.00149638 | 0 | 0 | 0.002262314 | 0.001652983 | −0.00082496 | 0.001807011 |
| 26459 | Slc27a5 | 0.000125029 | 0 | 0 | −0.00154962 | −0.00115222 | −0.00041147 | 0 |
| 53422 | Ybx2 | −0.00015835 | 0 | 0 | −0.00132624 | −0.00143672 | −0.000809 | 0 |
| 381413 | Gpr176 | 0 | 0 | 0 | 0.002520356 | 0.001513128 | 0 | 0 |
| 19122 | Prnp | 0.001422922 | 0 | 0 | 0.001557817 | 0.001461533 | 0.001620564 | 0.002084989 |
| 19264 | Ptprc | 0.000771329 | 0 | 0 | 0.001127795 | 0.001664758 | 0.001458783 | 0.001404223 |
| 84682 | Cox4l2 | 0 | 0 | 0 | −0.00193592 | −0.00156955 | 0 | 0 |
| 109828 | C7 | 0 | 0 | 0 | 0.001590617 | 0.000297232 | 0 | 0 |
| 320557 | Fam169a | 0 | 0 | 0 | 0.002372562 | 0.001450424 | 0 | 0 |
| 14125 | Fcer1a | −0.00135024 | 0 | 0 | 0.001271645 | 0.001486968 | −0.00058507 | 0 |
| 109731 | Maob | 0.000792368 | 0 | 0 | 0.002574591 | 0.001585815 | 0.001144699 | 0.001964808 |
| 72119 | Tpx2 | −0.00030043 | 0 | 0 | 0.0004002 | −0.00101001 | −0.00152863 | −0.00213829 |
| 65221 | Slc15a3 | 2.58E−05 | 0 | 0 | 0.001531379 | 0.001291081 | 0.001077404 | 0.002135461 |
| 66060 | Cystm1 | 0.00083801 | 0 | 0 | 0.001124714 | 0.001375623 | 0.001410577 | 0.002026048 |
| 52588 | Tspan14 | −0.00026629 | 0 | 0 | −0.00126221 | −0.00137457 | −0.00059342 | −0.00191821 |
| 66938 | Sh3d21 | −0.00131469 | 0 | 0 | −0.00226171 | −0.00136163 | −0.00032467 | −0.00209915 |
| 69809 | 1810046K07Rik | 0.000878143 | 0 | 0 | 0 | 0.001317418 | 0.00159729 | 0 |
| 14163 | Fgd1 | −0.00086011 | 0 | 0 | −0.0022385 | −0.00117982 | −0.00157648 | −0.00214941 |
| 218763 | Lrrc3b | −2.48E−05 | 0 | 0 | −0.00263883 | −0.00139812 | −0.00067877 | 0 |
| 227627 | Obp2a | −0.00161919 | 0 | 0 | 0 | 0 | 0.001124542 | 0 |
| 50765 | Tfr2 | 0.001026292 | 0 | 0 | 0.003310759 | 0.001699662 | 0.001255616 | 0.001859367 |
| 12269 | C4bp | 0.00169639 | 0 | 0 | 0.001861021 | 0.001444321 | 0.00141753 | 0.001400468 |
| 66771 | Gld4 | −0.00077204 | 0 | 0 | −0.00180806 | −0.00135802 | −0.00077487 | −0.00201288 |
| 235527 | Plscr4 | 0.001340094 | 0 | 0 | 0.002891469 | 0.001638374 | 0.001616075 | 0.002101135 |
| 70747 | Tspan2 | −0.00048193 | 0 | 0 | −0.0015031 | −0.00168276 | −0.00069493 | −0.00178183 |
| 17314 | Mgmt | 0.001315546 | 0 | 0 | −0.00039674 | 0.001465114 | 0.001571519 | 0.0003 |
| 20620 | Plk2 | 0.001871322 | 0 | 0 | 0.002350172 | 0.001606308 | 0.001508442 | 0.001610729 |
| 16818 | Lck | 0.000973466 | 0 | 0 | 0.001184993 | 0.001542635 | 0.001242511 | 0.001768553 |
| 13114 | Cyp3a16 | −0.00051106 | 0 | 0 | 0 | 0 | 0.001712956 | 0 |
| 71753 | Tmprss6 | −0.00124417 | 0 | 0 | −0.00149752 | −0.00045821 | −0.00082738 | 0 |
| 208677 | Creb3l3 | 0.001631959 | 0 | 0 | 0.002607875 | 0.00035706 | −1.75E−05 | −0.00032435 |
| 384783 | Irs2 | 0.000651864 | 0 | 0 | 0.000938495 | −0.00041205 | 0.001037703 | 0.000971832 |
| 239463 | Fam83a | −0.00093646 | 0 | 0 | 0 | 0 | −0.00106821 | 0 |
| 110033 | Kif22 | 0.001806413 | 0 | 0 | 0.000729967 | 2.11E−05 | 0.001127093 | 0.000252535 |
| 17842 | Mup3 | −0.00131548 | 0 | 0 | 0 | −0.00051396 | 0.00064921 | 0 |
| 22287 | Scgb1a1 | 0.000918585 | 0 | 0 | 0 | −0.00103586 | 0.000780032 | 0 |
| 68732 | Carmil1 | −0.00177205 | 0 | 0 | −0.0006209 | −0.00044508 | −0.00137106 | −0.00089861 |
| 100039008 | Mup10 | 0 | 0 | 0 | 0 | 0 | 0.001625665 | 0 |
| 18636 | Cfp | 0.001708787 | 0 | 0 | 0.000993313 | 0.000326166 | 0.000754479 | 0.000343128 |
| 16523 | Kcnj8 | −0.00092697 | 0 | 0 | −0.00153494 | −0.0010178 | −0.00158259 | −0.00141368 |
| 30839 | Fbxw5 | 0.000871534 | 0 | 0 | −0.00038918 | −0.00013459 | 0.001771671 | 0.001144039 |
| 75974 | Dock11 | 0.001547138 | 0 | 0 | 0.000205156 | 0.000481171 | 0.001224866 | −0.0002321 |
| 56708 | Clcf1 | −0.00060601 | 0 | 0 | −0.00017802 | 0.000328953 | −0.0011301 | 0 |
| 103988 | Gck | −0.00097325 | 0 | 0 | −0.00095035 | 4.57E−05 | −0.00145789 | −0.00161919 |
| 71982 | Snx10 | −0.00177776 | 0 | 0 | 0.00163286 | 0.000960477 | −0.00120657 | 0.000923067 |
| 102626 | Mapkapk3 | −0.00140228 | 0 | 0 | −0.00112848 | 0.000600165 | −0.00035076 | 0.000352577 |
| 15006 | H2-Q1 | 0.001722251 | 0 | 0 | 0 | 0 | 0.001490941 | 0 |
| 13419 | Dnase1 | 0.001645673 | 0 | 0 | 0.000274682 | −0.00041502 | 0.001501319 | 0.000223865 |
| 230784 | Sesn2 | 0.000991643 | 0 | 0 | 0.002199313 | 0.000837521 | 0.000902105 | 0.001560524 |
| 68371 | Pbld1 | 0.000332089 | 0 | 0 | 0.000190479 | −0.00024845 | 0.001132931 | 0 |
| 546336 | Prrg1 | 0.00155514 | 0 | 0 | 0.001149234 | 0.001076644 | 0.001487247 | 0.000806872 |
| 11425 | Apoc4 | 0.001882722 | 0 | 0 | 0.001757941 | 0.001035288 | 0.00144381 | 0 |
| 16364 | Irf4 | 0.001289627 | 0 | 0 | 0.001126212 | 0.000190194 | 0.001263598 | 0.000166749 |
| 320292 | Rasgef1b | −3.55E−07 | 0 | 0 | −0.00047125 | −0.00043185 | 0.001174846 | 0.000667178 |
| 337924 | Cyp3a44 | −0.00148142 | 0 | 0 | 0 | 0 | −0.00108305 | 0 |
| 229722 | Blapor1 | 0.000258242 | 0 | 0 | 0.000960257 | 0.000408432 | −0.00045368 | 0 |
| 17843 | Mup4 | −0.00176885 | 0 | 0 | 0 | 0 | −0.00018352 | 0 |
| 23960 | Qas1g | 0.000618958 | 0 | 0 | 0 | 0 | 0.000624733 | 0 |
| 68195 | Rnaset2b | 0.001731838 | 0 | 0 | 0.002691254 | 0.000830407 | 0.0016703 | 0 |
| 240186 | Zfp438 | 0.001714882 | 0 | 0 | 0.003226732 | 0.001145079 | 0.001409595 | 0.001487816 |
| 56386 | B4galt6 | 0.00138537 | 0 | 0 | −0.00023332 | −0.00018614 | 0.001134332 | 2.55E−05 |
| 70261 | Chp2 | −0.00123288 | 0 | 0 | −0.00156644 | −0.0004086 | −0.00074134 | −0.00020097 |
| 16469 | Jrk | −0.00013077 | 0 | 0 | −0.00041797 | 0.000280753 | −0.00079881 | −0.00043031 |
| 22445 | Xlr3a | −0.00032726 | 0 | 0 | 0 | 0 | 0.000578473 | 0 |
| 320924 | Ccbe1 | 0.000978187 | 0 | 0 | 0.001659202 | 0.000174663 | 0.000838187 | 0.001990829 |
| 70123 | Nbdy | 0.000187945 | 0 | 0 | 0.003295277 | 0.000418688 | 5.58E−05 | 0.000167242 |
| 13118 | Cyp4a12b | 0.001701184 | 0 | 0 | 0 | 0 | 0.001070645 | 0 |
| 13098 | Cyp2c39 | 0.000228078 | 0 | 0 | −0.00189267 | −0.00111767 | −0.0007487 | 0 |
| 17395 | Mmp9 | −8.34E−05 | 0 | 0 | 0.000256455 | 0.000714568 | −0.0001084 | 0.001191596 |
| 69454 | Clic3 | −0.00073934 | 0 | 0 | −0.00050845 | −0.00019073 | −0.0007269 | −3.45E−05 |
| 100705 | Acacb | −0.00019047 | 0 | 0 | 0.000184637 | 0.001018941 | 0.000396585 | 0.000266137 |
| 17069 | Ly6e | −0.00030106 | 0 | 0 | 0.000862083 | 0.000413591 | 0.001033622 | 0.001738473 |
| 380839 | Serpinb1c | 0 | 0 | 0 | 0 | 0 | −0.00119378 | 0 |
| 74365 | Lonrf3 | 0.000669283 | 0 | 0 | −0.00041399 | 0.000650931 | 0.000628344 | 0 |
| 16765 | Stmn1 | 0.000380716 | 0 | 0 | 0.000642366 | −0.00020021 | 0.000119331 | −0.000741 |
| 381218 | Spata6l | 0.001183267 | 0 | 0 | 0.001685808 | 0.000487519 | 0.001580294 | 0.000601526 |
| 71780 | Isyna1 | 0.000717548 | 0 | 0 | −0.00151999 | −0.00037242 | 0.000731315 | −0.00053777 |
| 27981 | Rsrp1 | −0.00145164 | 0 | 0 | −0.00162914 | −0.00059474 | −0.00163004 | −0.00106212 |
| 71755 | Dhdh | −0.00108862 | 0 | 0 | −0.00247396 | −0.00036113 | −0.000648 | 0.000246024 |
| 14289 | Fpr2 | −0.00118005 | 0 | 0 | 0 | 0 | −0.00125403 | 0 |
| 66569 | Gdpd1 | −0.0008158 | 0 | 0 | 0.00279463 | 0.00035439 | −0.00030897 | 0.001450998 |
| 72082 | Cyp2c55 | −0.00180927 | 0 | 0 | −0.00185408 | 0.000252141 | −0.00175956 | 0 |
| 72361 | Ces2g | −0.00072831 | 0 | 0 | −0.00154234 | −0.00092395 | −0.00172147 | −0.00124711 |
| 16145 | Igtp | 0.000109006 | 0 | 0 | −0.00201293 | 0.000182948 | 0.001090297 | 0.000757831 |
| 59608 | Sec24d | −0.00120776 | 0 | 0 | 0.000217493 | −0.00017282 | 0.00142395 | −0.00093147 |
| 107448 | Unc5a | 0.001567027 | 0 | 0 | 0.000454363 | 0.000738711 | 0.001657138 | 0.000290195 |
| 66253 | Aig1 | −0.00192983 | 0 | 0 | −0.00089633 | 0.000407109 | −0.00114292 | 4.32E−05 |
| 330406 | B4galnt3 | 0 | 0 | 0 | 0.000103889 | −0.00010569 | −0.00098797 | 0 |
| 269346 | Slc28a2 | 0.000331841 | 0 | 0 | 0.000560018 | −0.00064711 | 0.000340767 | 0.000446978 |
| 628813 | Gm11437 | 1.92E−06 | 0 | 0 | 0 | 0.000852476 | −0.00099064 | 0 |
| 13730 | Emp1 | −0.00043555 | 0 | 0 | 0.001720239 | 0.000159082 | −0.00050366 | 0.001276716 |
| 13094 | Cyp2b9 | 0.001250264 | 0 | 0 | 0 | 0 | 0.001621475 | 0 |
| 16326 | Inhbe | −5.40E−05 | 0 | 0 | 0.001102409 | 0.001083886 | −0.00058538 | 0 |
| 12583 | Cdo1 | −1.23E−05 | 0 | 0 | −0.00020222 | 1.58E−05 | −0.00040035 | −0.00041524 |
| 18588 | Pde6g | 0.001403739 | 0 | 0 | −0.00082583 | −0.00015519 | 0.001691182 | 0 |
| 231821 | Adap1 | 0.001251304 | 0 | 0 | −3.57E−05 | −0.00087011 | 0.001185566 | −0.00061415 |
| 101831 | Faap24 | 0.001381816 | 0 | 0 | −4.13E−05 | −0.00063274 | 0.001353632 | −0.00065648 |
| 207259 | Zbtb7c | 0.000653047 | 0 | 0 | −0.00034631 | 0.000222609 | −0.00019607 | −3.17E−05 |
| 71699 | Slc41a3 | 0.000110833 | 0 | 0 | 0.000496619 | −0.0006411 | 0.00041625 | −0.00040033 |
| 74694 | Tbc1d30 | 0.000772811 | 0 | 0 | 0.002860243 | 0.000125865 | 0.000785051 | 0.000271991 |
| 18828 | Plscr2 | 0.001295968 | 0 | 0 | 0.003028851 | 0.001263407 | 0.001411719 | 0.001632408 |
| 55963 | Slc1a4 | −0.00150878 | 0 | 0 | −0.00050274 | −0.00083892 | −0.00104011 | 0.000134903 |
| 70370 | Fbln7 | 0.001397265 | 0 | 0 | 0.001343863 | 0.001059381 | 0.001574233 | 0.001707204 |
| 217517 | Stxbp6 | 0.001677529 | 0 | 0 | 0.001781307 | 0.000319582 | 0.000757997 | 0.00032047 |
| 16325 | Inhbc | −0.00134619 | 0 | 0 | 0.002681836 | 0.000554012 | −0.00144184 | 0 |
| 56741 | Igdcc4 | −3.20E−05 | 0 | 0 | −0.00126208 | −0.00031538 | 0.001240587 | 0.000178875 |
| 245050 | Gask1a | 0.001105719 | 0 | 0 | 0.001162268 | 0.000418409 | −0.00017288 | 0 |
| 11944 | Atp4a | −0.00114328 | 0 | 0 | 0 | −4.37E−05 | −0.00139886 | 0 |
| 242316 | Gdf6 | 0.00111436 | 0 | 0 | 0 | 0 | 0.000932311 | 0 |
| 98660 | Atp1a2 | −0.00104087 | 0 | 0 | 9.11E−05 | 0.000326651 | −0.00142637 | −0.00104072 |
| 18242 | Oat | 2.83E−05 | 0 | 0 | −5.70E−05 | −5.38E−05 | −0.00011574 | 0.000816597 |
| 213019 | Pdlim2 | −0.00152169 | 0 | 0 | −0.00186692 | −0.00085968 | −0.00048215 | −0.00116486 |
| 215335 | Slc36a1 | 0.00038977 | 0 | 0 | 0.000288399 | −0.00093255 | 0.00021208 | −0.00111248 |
| 17035 | Lxn | 0.000613326 | 0 | 0 | −0.00074988 | −0.00050147 | 0.000237772 | −0.00026539 |
| 668212 | Efr3b | 0.000738657 | 0 | 0 | 0.001677302 | 0.000435344 | −1.00E−04 | 0.0004533 |
| 15186 | Hdc | 0.00076795 | 0 | 0 | −0.00060003 | 0.000453621 | 0.000407013 | 0.000432272 |
| 66506 | Psmg3 | 0.001559811 | 0 | 0 | 0.00070824 | 0.000390064 | 0.00055629 | 9.97E−05 |
| 230903 | Fbxo44 | −0.00145181 | 0 | 0 | −0.00026661 | 1.41E−05 | −0.00065443 | 0.000367159 |
| 71693 | Colec11 | −0.00112018 | 0 | 0 | 8.43E−05 | 0.000309127 | −0.00098508 | 0.000588262 |
| 382053 | Ces3a | −0.0001431 | 0 | 0 | 0 | 0 | −0.00034152 | 0 |
| 21822 | Tgtp1 | −0.00067396 | 0 | 0 | −0.00196332 | −0.00108609 | −0.0004303 | 0 |
| 22029 | Traf1 | −0.00085805 | 0 | 0 | −0.00149405 | −0.00082477 | −0.00095121 | −0.00094747 |
| 381531 | Mup21 | −0.00055367 | 0 | 0 | 0 | 0 | −8.96E−05 | 0 |
| 665563 | Mthfd2l | 0.00053371 | 0 | 0 | 0.00123942 | 0.000200781 | 0.000312436 | 0.000718073 |
| 232934 | Mypop | 0.001412853 | 0 | 0 | 0.001356587 | 0.000635499 | 0.000826865 | 0.000756512 |
| 77864 | Ypel2 | 0.000573326 | 0 | 0 | 0.003398205 | 0.001002636 | 0.000386786 | 0.001434972 |
| 171281 | Acot3 | −0.00095285 | 0 | 0 | 0 | −0.00073878 | −0.00124827 | 0 |
| 30843 | Fbxl12 | 0.001444788 | 0 | 0 | 0.001329676 | 0.00058784 | 0.001210556 | 0.000432392 |
| 76074 | Gbp8 | −0.00029486 | 0 | 0 | 6.11E−05 | 0.000182152 | −0.00098143 | 0 |
| 327957 | Scimp | 0.001445738 | 0 | 0 | 0 | 0.000208502 | 0.000795348 | 0 |
| 17922 | Myo7b | 0.001336624 | 0 | 0 | −0.0014697 | 0.000318241 | 0.001777436 | 0 |
| 235330 | Ttc12 | 0.00063814 | 0 | 0 | 0.002791835 | 0.001208756 | 0.00095388 | 0.001968305 |
| 83675 | Bicc1 | −1.44E−05 | 0 | 0 | −0.00024509 | −0.00048807 | −0.00059281 | −0.00020938 |
| 16855 | Lgals4 | 0.000773254 | 0 | 0 | 0.002054991 | 0.000812716 | 0.00056803 | 0.001306739 |
| 67378 | Bbs2 | −0.00102478 | 0 | 0 | 0.000285446 | 0.000432005 | −0.00105278 | 0.000401181 |
| 16571 | Kif4 | −0.00161314 | 0 | 0 | −0.00157633 | −0.00111787 | −0.00162641 | −0.00192662 |
| 54167 | Icos | −0.00062362 | 0 | 0 | 0 | −0.00077924 | −5.35E−05 | 0 |
| 56715 | Rabgef1 | 0.000806674 | 0 | 0 | 2.87E−05 | −0.0001044 | 0.000862064 | 2.45E−05 |
| 13108 | Cyp2g1 | −0.00191657 | 0 | 0 | 0 | −0.00057249 | −0.00098814 | 0 |
| 18048 | Klk1b4 | 0.001625193 | 0 | 0 | 0 | 0.000712814 | 0.000240072 | 0 |
| 241303 | Fam78a | −0.00087686 | 0 | 0 | −0.00103727 | −0.00136351 | −0.00088336 | −0.00112385 |
| 14247 | Fli1 | −0.00112438 | 0 | 0 | −0.0028355 | −0.00105808 | −0.00095677 | −0.00121515 |
| 329650 | Med12l | −0.00176071 | 0 | 0 | −0.0027382 | −0.00117868 | 0.00023076 | −0.00101223 |
| 72445 | Prr5l | −0.0017314 | 0 | 0 | 0.000442239 | −9.84E−06 | −0.00055892 | 0.000115812 |
| 26380 | Esrrb | 0.000807299 | 0 | 0 | 0.000667398 | 8.86E−05 | −5.42E−05 | −0.00052291 |
| 319582 | Trmt9b | −0.00107247 | 0 | 0 | −0.00030008 | −6.29E−05 | −0.00047031 | 1.39E−05 |
| 11481 | Acvr2b | 0.001778662 | 0 | 0 | 0.001796983 | 2.59E−05 | 0.001229691 | 0.000140358 |
| 72289 | Malat1 | −0.00044635 | 0 | 0 | −0.00026889 | −0.0002114 | −0.00101519 | −0.00100826 |
| 68172 | Rpl39l | 0.000436521 | 0 | 0 | 0 | 0 | 0.001482928 | 0 |
| 12623 | Ces1g | 0.000440424 | 0 | 0 | −0.00118499 | −7.59E−05 | −0.00070424 | 0 |
| 23948 | Mmp17 | −0.0006628 | 0 | 0 | −0.00042379 | −0.00090585 | −0.00104648 | −0.0005685 |
| 330723 | Htra4 | −0.00091942 | 0 | 0 | −0.00290005 | −0.00036164 | 9.09E−05 | 0 |
| 329910 | Acct11 | −0.00143995 | 0 | 0 | −0.00228168 | −0.00073795 | −0.00040764 | 0.000445586 |
| 54608 | Abhd2 | 0.000390608 | 0 | 0 | 0.001224584 | −0.00014224 | 0.001064496 | 0.001011479 |
| 58992 | F12 | 0.001755627 | 0 | 0 | 0.002738606 | 0.000158933 | 0.001108899 | 0 |
| 235047 | Zfp809 | 0.000241601 | 0 | 0 | −0.00053748 | −0.00014859 | 0.000460679 | 0.000394109 |
| 24004 | Rai2 | −0.00024177 | 0 | 0 | −0.00213699 | 0.000192197 | 0.000396341 | 0.001304759 |
| 17929 | Myom1 | −0.00032476 | 0 | 0 | 0.001697998 | 0.000634443 | −0.00112311 | 0.000671291 |
| 70454 | Cenpl | −0.0017285 | 0 | 0 | 0.000141866 | 7.25E−05 | −0.00062886 | 0.000169821 |
| 18302 | Oit3 | −0.00093936 | 0 | 0 | −0.00131939 | 0.000173177 | −0.0006603 | 0 |
| 14939 | Gzmb | 0.000347457 | 0 | 0 | 0 | 0 | −0.00025609 | 0 |
| 60507 | Qtrt1 | −0.00147195 | 0 | 0 | 0.000249299 | −0.00038467 | −0.00142702 | −0.0007986 |
| 327959 | Xaf1 | −0.00171895 | 0 | 0 | 0.00038477 | 0.000462134 | −0.00110636 | 0.000990869 |
| 232933 | Ccdc61 | −0.00074024 | 0 | 0 | −0.00065639 | −0.00061119 | −0.00073394 | −0.00053367 |
| 63953 | Dusp10 | −0.00147399 | 0 | 0 | −0.00174466 | −0.00068842 | −0.00137766 | −0.00110194 |
| 17768 | Mthfd2 | 0.000591968 | 0 | 0 | −0.00065829 | −0.00053993 | 0.000207048 | −0.00055141 |
| 15200 | Hbegf | 0.001062302 | 0 | 0 | 0.001541802 | −0.00078696 | 0.000535919 | −0.00073112 |
| 257635 | Sdsl | −0.00170891 | 0 | 0 | −0.00014984 | 0.000857567 | −0.00144571 | 0.001071958 |
| 272158 | Poln | 0.001256554 | 0 | 0 | −0.00071012 | −0.00016873 | 0.001442609 | 0.000353813 |
| 212439 | AA986860 | 0.000460155 | 0 | 0 | 0.002025047 | 0.0004621 | 0.000434708 | 0.001627748 |
| 56356 | Gltp | −0.00015635 | 0 | 0 | −4.67E−05 | 0.000305555 | 0.000710534 | 0.001028584 |
| 11555 | Adrb2 | −0.00154511 | 0 | 0 | −0.00297039 | −0.00124789 | −1.29E−05 | −0.00162227 |
| 16981 | Lrrn3 | 0.000472175 | 0 | 0 | −0.00140656 | 0.000404215 | −0.00015813 | −0.00016443 |
| 246103 | Atxn7 | 0.001461132 | 0 | 0 | 0.000916344 | 0.000751174 | 0.001525538 | 0.001014944 |
| 93700 | Pcdhgb2 | 0 | 0 | 0 | 0.000765598 | 0.000279677 | −0.00102313 | 0 |
| 17709 | COX2 | −0.00049794 | 0 | 0 | 0 | 0 | 0.000538508 | 0 |
| 71132 | Cabyr | 0.000393959 | 0 | 0 | 0.000467706 | 0.000481975 | −0.00099837 | −0.00017679 |
| 117606 | Boc | −0.00118861 | 0 | 0 | −0.00099095 | −8.66E−05 | −0.0013411 | −0.00066043 |
| 74747 | Ddit4 | −0.0012606 | 0 | 0 | −0.00255482 | −0.00118351 | 0.001447914 | 0.00072109 |
| 27362 | Dnajb9 | 3.80E−05 | 0 | 0 | −0.0005174 | 0.000609828 | −0.00015671 | 6.89E−05 |
| 69034 | Nupr1l | 0.001170843 | 0 | 0 | 0 | 0.00051828 | 0.001507078 | 0 |
| 214547 | She | 0.001278744 | 0 | 0 | 0.001555488 | 0.000152525 | 0.000979615 | 0.000223153 |
| 215653 | Rassf2 | −0.00075795 | 0 | 0 | −0.00012321 | −0.00025529 | 7.21E−06 | −3.26E−05 |
| 212391 | Lcor | −0.00023108 | 0 | 0 | −0.00124704 | 9.11E−05 | −0.00056821 | −0.00057802 |
| 101543 | Wtip | −0.00084204 | 0 | 0 | −0.00114335 | 1.82E−05 | −0.0005524 | 0.000782932 |
| 19700 | Rem1 | −0.00063677 | 0 | 0 | −0.00244702 | −0.00097136 | 0.000130466 | −0.00084907 |
| 240869 | Zbtb37 | −0.0014681 | 0 | 0 | −0.0019643 | −0.00021132 | −0.00084255 | −0.00013239 |
| 17844 | Mup5 | −0.0009855 | 0 | 0 | 0 | 0 | 9.07E−05 | 0 |
| 27214 | Dbf4 | −3.04E−05 | 0 | 0 | −0.00021187 | −0.00069157 | 0.000443957 | −0.00023366 |
| 319924 | Apba1 | 0.001457153 | 0 | 0 | 0.000610436 | 0.000230521 | 0.00081413 | −0.00103295 |
| 217328 | Myo15b | −0.00120454 | 0 | 0 | −0.00268825 | −0.00072524 | −0.00065133 | 0 |
| 68201 | Ccdc34 | 0.001163656 | 0 | 0 | 0.001795466 | 0.000257045 | 0.000741953 | 0.000142537 |
| 18119 | Nodal | 0.000657506 | 0 | 0 | 0.001986579 | 0.000375974 | 0.001075233 | 0 |
| 12836 | Col7a1 | 0.000250494 | 0 | 0 | −0.00041065 | −0.00043487 | 0.000830914 | 0.000268011 |
| 13436 | Dnmt3b | −0.00136297 | 0 | 0 | 0.000618197 | −0.00016687 | −0.00178659 | 0.000160705 |
| 209176 | Ido2 | −0.00081651 | 0 | 0 | 0 | −0.00028417 | −0.00166722 | 0 |
| 240327 | Gm4951 | −0.00012886 | 0 | 0 | 0.002931839 | 0.001173147 | −0.00106988 | 0.001687108 |
| 52850 | Sgsm1 | −0.00069962 | 0 | 0 | 0.00022044 | 0.00025628 | −0.0002982 | 0.000423106 |
| 192136 | Sugct | −8.56E−05 | 0 | 0 | 0.000740343 | 0.000590012 | −0.00034753 | 3.51E−05 |
| 236930 | Ercc6l | 0.001025636 | 0 | 0 | −0.0008196 | −0.00026879 | 0.001428233 | 0 |
| 78317 | Ccdc88b | −0.00129385 | 0 | 0 | −0.00316263 | −0.00093183 | −0.00122629 | −0.00082535 |
| 15248 | Hic1 | −0.00133069 | 0 | 0 | 0.000208423 | 0.000179251 | −0.00173217 | −0.00016732 |
| 13086 | Cyp2a4 | 0 | 0 | 0 | 0 | 0 | 0.000763288 | 0 |
| 72480 | Tspyl4 | −0.00110744 | 0 | 0 | −0.00041319 | −0.00082324 | −0.00083745 | −0.00088893 |
| 668303 | Kif26a | −0.00127194 | 0 | 0 | −0.00111743 | −0.00025233 | −0.00115413 | −0.00031145 |
| 217333 | Trim47 | 0.00028409 | 0 | 0 | 0.001477872 | 0.000732081 | −0.00107219 | 8.50E−05 |
| 232333 | Slc6a1 | −0.00191636 | 0 | 0 | −0.00077746 | −0.00118731 | −0.00154132 | 0 |
| 70061 | Sdr9c7 | 0.000368513 | 0 | 0 | 0.001712698 | 0.000728773 | −0.000238 | 0 |
| 17718 | ND3 | −0.00024564 | 0 | 0 | 0 | 0 | −0.00014771 | 0 |
| 14461 | Gata2 | 0.000796007 | 0 | 0 | 0.001909511 | 0.000937227 | −0.00026944 | 0.001215059 |
| 58220 | Pard6b | −0.00012216 | 0 | 0 | 0.000827199 | 0.000694881 | −0.00068963 | −0.00024549 |
| 140859 | Nek8 | −0.00115965 | 0 | 0 | −0.00131872 | −0.000473 | −0.00045393 | −0.00073407 |
| 320977 | A330023F24Rik | 0.001273303 | 0 | 0 | 0.002834008 | 0.001471754 | 0.000870595 | 0 |
| 107566 | Art2bp | 0.000531248 | 0 | 0 | 0.000561873 | −0.00010481 | −9.02E−05 | −0.00029821 |
| 69535 | Ten1 | 0.000898656 | 0 | 0 | −0.00108179 | 0.000196104 | 0.00104664 | −0.00085494 |
| 27062 | Cadps | −0.0001797 | 0 | 0 | 0.000679027 | 0.001137203 | 0.000150401 | 0 |
| 14469 | Gbp2 | −0.00095453 | 0 | 0 | −0.00054291 | 0.000639884 | −0.00082139 | 0.000508702 |
| 18710 | Plk3r3 | 0.000680306 | 0 | 0 | 0.002211799 | 0.000703072 | 0.000558861 | 0.001356948 |
| 22160 | Twist1 | −0.00141435 | 0 | 0 | −0.00206686 | −0.00044424 | −0.00147073 | 0 |
| 70024 | Mcm10 | 0.000765195 | 0 | 0 | 0.002761631 | −0.00056338 | 0.001293423 | 0.001735573 |
| 100504714 | Gm16793 | 0 | 0 | 0 | 0 | 0 | 0.001057477 | 0 |
| 105837 | Mtbp | −0.00140736 | 0 | 0 | 0.000271624 | 0.000560103 | −0.00101986 | 0.000952185 |
| 245622 | Fam199x | −0.00118336 | 0 | 0 | −0.00071615 | −0.00024965 | −0.00146874 | −0.00129312 |
| 68910 | Zfp467 | −0.00067144 | 0 | 0 | 0.001052958 | 0.001323582 | −0.00060445 | 0.001089289 |
| 76044 | Ncapg2 | 0.000694387 | 0 | 0 | 0.002138622 | 0.000835123 | 0.000652665 | 6.96E−05 |
| 11761 | Aox1 | −0.00013612 | 0 | 0 | −0.00086216 | 0.001243586 | −0.00047065 | 0.001172741 |
| 353310 | Zfp703 | −0.00062797 | 0 | 0 | 0.000369059 | 0.00068971 | −0.00058329 | 0.000838982 |
| 20908 | Stx3 | −0.0006522 | 0 | 0 | 0.001261729 | 0.000420388 | 0.00071722 | 0.000442729 |
| 71151 | Eri2 | −0.00018992 | 0 | 0 | −0.00214649 | −0.00112169 | 0.000186295 | −0.00119768 |
| 19057 | Ppp3cc | 0.00082294 | 0 | 0 | 0.002315083 | 0.001272701 | 0.001097761 | 0.001414594 |
| 77739 | Adamtsl1 | −0.00093307 | 0 | 0 | −0.00208621 | −0.00030768 | −0.00032014 | −0.00012358 |
| 22702 | Zfp42 | −0.00137911 | 0 | 0 | 0 | 0 | −0.00125546 | 0 |
| 224840 | Treml4 | −0.00100661 | 0 | 0 | −0.00155946 | −0.00063784 | 7.77E−05 | −0.00051335 |
| 15040 | H2-T23 | 0.000708612 | 0 | 0 | −0.00034954 | 0.00045206 | 0.001420987 | 0.001111435 |
| 231070 | Insig1 | −0.00071852 | 0 | 0 | −0.00016983 | 0.000657401 | 0.000469443 | 0.000167179 |
| 16196 | Il7 | 0.000861867 | 0 | 0 | 0.000339117 | 0.00102473 | 0.001014177 | 0.001070729 |
| 70574 | Cpm | 0.000531917 | 0 | 0 | 6.28E−05 | −0.00043115 | 0.00157786 | −0.00012117 |
| 14913 | Guca1a | −0.00034799 | 0 | 0 | 0 | 0 | 5.07E−05 | 0 |
| 96935 | Susd4 | 0.000680981 | 0 | 0 | −0.00013863 | 0.000554775 | −0.00119901 | −0.00095442 |
| 58185 | Rsad2 | −0.00136682 | 0 | 0 | −0.00036596 | 0.001120039 | −0.00024005 | 0.001324303 |
| 67391 | Fundc2 | 0.001074017 | 0 | 0 | 0.002908534 | 0.00107545 | 0.001585314 | 0.001383823 |
| 21665 | Tdg | −0.00071703 | 0 | 0 | −0.00155219 | −0.0009556 | 0.000410412 | −0.00052161 |
| 17218 | Mcm5 | −0.00043967 | 0 | 0 | −0.00234824 | −0.00135933 | −0.00099905 | −0.00205613 |
| 68628 | Fbxw9 | 0.000794776 | 0 | 0 | 0.002569852 | 0.000156828 | 0.000917524 | 0.001049476 |
| 211986 | Tmem18 | −0.00160534 | 0 | 0 | −0.00168724 | −0.00034565 | −0.00098563 | −0.00057192 |
| 108961 | E2f8 | −0.00078818 | 0 | 0 | −0.00111918 | −0.00035209 | −0.00163808 | 0 |
| 16011 | Igfbp5 | 0.001546469 | 0 | 0 | 0.000842045 | 0.00023959 | 0.000971107 | 0.000404187 |
| 246293 | Klhl8 | 0.001682161 | 0 | 0 | 0.000590806 | 0.00054912 | 0.001343528 | 0.000111528 |
| 73230 | Bmper | −0.00032978 | 0 | 0 | 0.00100405 | −0.00052268 | −0.00087241 | 0.000113288 |
| 56434 | Tspan3 | −0.00151298 | 0 | 0 | −0.00209489 | −0.00098962 | −0.00077567 | −0.00085519 |
| 333424 | A4gnt | 0.001871069 | 0 | 0 | 0 | 0 | 0.001404393 | 0 |
| 11302 | Aatk | −0.0003967 | 0 | 0 | −0.00277775 | −0.00080676 | 0.000170868 | 6.12E−06 |
| 69376 | Zpbp2 | −0.00182959 | 0 | 0 | 0 | −0.00026852 | −0.00156169 | 0 |
| 16000 | Igf1 | −0.00070888 | 0 | 0 | −0.00103788 | −0.00130553 | 0.001331984 | 0.000435667 |
| 226744 | Cnst | 4.81E−05 | 0 | 0 | 0.000857645 | 0.000276088 | −0.00037463 | 1.63E−05 |
| 108686 | Ccdc88a | −0.001901 | 0 | 0 | −0.00199043 | −0.00077785 | −0.00160274 | −0.00111947 |
| 28254 | Slco1a6 | −0.00110915 | 0 | 0 | 0 | −0.000916 | −0.00104498 | 0 |
| 23888 | Gpc6 | −0.00108789 | 0 | 0 | −0.00328094 | −0.00103437 | −0.00098718 | −0.00109086 |
| 11657 | Alb | 0.001386953 | 0 | 0 | 0.002643872 | 0.000403332 | 0.000300132 | −0.00029673 |
| 236149 | Slc22a26 | 0.001722182 | 0 | 0 | 0 | −0.00076124 | 0.000770026 | 0 |
| 14357 | Dtx1 | −0.00025855 | 0 | 0 | −0.00220264 | −0.00086947 | −0.00104682 | −0.00137597 |
| 23890 | Gpr34 | −9.22E−05 | 0 | 0 | 0.0019742 | 0.00026619 | 0.000306042 | 0.001221004 |
| 320139 | Ptpn7 | 0.000223869 | 0 | 0 | −0.00151631 | 0.000108504 | 0 | 0 |
| 19660 | Rbp2 | −0.00110282 | 0 | 0 | 0 | −0.00120332 | −0.00130081 | 0 |
| 57905 | Isy1 | −1.73E−05 | 0 | 0 | −0.00319608 | −0.00037537 | −8.05E−05 | −0.00078322 |
| 210321 | BC048679 | 0.000307281 | 0 | 0 | 0 | 0 | −0.00010589 | 0 |
| 320712 | Abi3bp | −0.00153492 | 0 | 0 | −0.00319235 | −0.0012009 | −0.00116524 | −0.00206568 |
| 83674 | Cnnm1 | −0.00027165 | 0 | 0 | 0.002350249 | 0.001429323 | 0.0004134 | 0 |
| 83453 | Chrdl1 | −0.00034848 | 0 | 0 | −0.00037969 | 0.000546748 | −0.00086436 | 0.000512359 |
| 56722 | Litaf | 0.000889958 | 0 | 0 | 0.002471379 | 0.001141578 | 0.001566997 | 0.001808705 |
| 104158 | Ces1d | −0.00015221 | 0 | 0 | 0.000679119 | 0.000533048 | 0.000567167 | 0.001489981 |
| 72281 | Sh2d4a | −0.0001607 | 0 | 0 | 0.002824211 | 0.000908816 | −0.0004314 | 0.00214271 |
| 14293 | Fpr1 | −0.00133387 | 0 | 0 | −0.002313 | −0.00094764 | −0.00174413 | 0 |
| 13056 | Cyb561 | 0.000490946 | 0 | 0 | −0.00310811 | −0.00147083 | 0.000607183 | −0.00134685 |
| 236604 | Plsd-ps1 | 0.001004609 | 0 | 0 | 0.00259696 | 0.001060193 | 0.001256321 | 0 |
| 380997 | Cyp2d12 | 0.000675668 | 0 | 0 | 0 | 0 | 0.00162187 | 0 |
| 246049 | Slc36a2 | −0.00044185 | 0 | 0 | −0.00264727 | −0.00123491 | −0.00036979 | −0.00094608 |
| 211945 | Plekhh1 | −0.00067104 | 0 | 0 | 0.001416119 | 0.000258723 | 0.000302675 | 0.001344766 |
| 12759 | Clu | 8.43E−05 | 0 | 0 | −0.00238966 | 0.000410234 | −0.00041917 | −0.00038526 |
| 13024 | Ctla2a | 0.000488477 | 0 | 0 | 0.002092292 | 0.000480649 | 0.001184997 | 0.001605214 |
| 11501 | Adam8 | 0.000647626 | 0 | 0 | −0.00160682 | 0.000783169 | 8.20E−05 | 0.000104511 |
| 239126 | C1qtnf9 | 0 | 0 | 0 | −0.00251731 | −0.00018753 | 0 | 0 |
| 98238 | Lrrc59 | −3.73E−05 | 0 | 0 | −0.00210918 | −0.0005756 | −0.00042792 | −0.00034974 |
| 236285 | Land3 | −0.00061271 | 0 | 0 | 0.001053835 | 0.000581116 | −0.00071835 | 0 |
| 107371 | Exoc6 | 0.000117416 | 0 | 0 | 0.002829485 | 0.000960863 | 0.000294392 | 0.00168849 |
| 14239 | Foxs1 | 0.000146307 | 0 | 0 | 0.00064248 | −8.97E−05 | 0.000457423 | 0.000271877 |
| 237175 | Adgrg2 | −0.00031044 | 0 | 0 | −0.00245811 | 0.000919377 | 0.000325813 | 0.000749479 |
| 12143 | Blk | 0.001202061 | 0 | 0 | 0.002663729 | 0.001471606 | 0.001072068 | 0.001211753 |
| 230991 | Fndc10 | 0 | 0 | 0 | 0.002115439 | 0.001151608 | 0.001041922 | 0.001774676 |
| 53897 | Gal3st1 | 0.000310995 | 0 | 0 | 0.001803545 | 0.000543635 | 0.000758829 | 0.000799599 |
| 16403 | Itgs6 | −0.00113708 | 0 | 0 | −0.00348494 | −0.00109328 | −0.00035738 | −0.00013141 |
| 109151 | Chd9 | −0.00051979 | 0 | 0 | −0.00168931 | −0.00045088 | −0.00057101 | −0.00082237 |
| 29816 | Hip1r | −2.04E−05 | 0 | 0 | 0.00320074 | 0.000512794 | −0.00015941 | 0.001641366 |
| 15439 | Hp | 0.000411359 | 0 | 0 | −0.00184542 | −0.00013333 | −0.0001404 | −0.00090551 |
| 20556 | Slfn2 | 0.000110764 | 0 | 0 | −0.00276872 | −0.00069635 | −0.00023066 | −0.0014057 |
| 71242 | Spata24 | −0.00046791 | 0 | 0 | −0.00116989 | 0.000502101 | 0.00031267 | 0.001441041 |
| 215418 | Csrnp1 | 0.000558935 | 0 | 0 | 0.001972506 | 7.42E−05 | 0.00099063 | 0.000427378 |
| 103768 | Tubg2 | 9.73E−05 | 0 | 0 | −0.00048433 | −0.00018377 | −0.00070426 | −0.00049331 |
| 19419 | Rasgrp1 | −0.00043108 | 0 | 0 | −0.00261856 | −0.0002127 | −0.00056674 | −0.00010553 |
| 106840 | Unc119b | 0.0005284 | 0 | 0 | 0.00165304 | 0.000339761 | 0.000653154 | 0.000541366 |
| 14071 | F9 | 0.001619887 | 0 | 0 | 0 | 0.000992102 | 0.001616878 | 0 |
| 23965 | Tenm3 | 0.000132988 | 0 | 0 | 0.001870305 | 0.000866165 | −0.00028035 | 0.000481463 |
| 12504 | Cd4 | 0.000582244 | 0 | 0 | −0.00262437 | 0.000221042 | 9.35E−06 | −0.0014384 |
| 67739 | Slc48a1 | −0.00033821 | 0 | 0 | −0.00289388 | −0.00041179 | −0.00030255 | −0.00090123 |
| 319909 | Ism1 | 0 | 0 | 0 | −0.0034633 | −0.00103746 | 0 | 0 |
| 12402 | Cbl | 0.000820333 | 0 | 0 | −0.00137773 | −0.00010765 | 0.000400173 | −0.00064354 |
| 628308 | Zfp970 | 0 | 0 | 0 | 0.002985122 | 0.001180418 | 0.001500313 | 0 |
| 53972 | Ngef | 0.000760026 | 0 | 0 | −0.00054132 | −0.00057967 | −0.00027845 | −0.00081459 |
| 279028 | Adamts13 | −0.00159576 | 0 | 0 | −0.00174761 | −0.00027057 | −0.00116535 | 0 |
| 218629 | Dhx29 | 0.000174656 | 0 | 0 | −0.00349549 | −0.00076759 | −0.00031053 | −0.00182745 |
| 17427 | Mns1 | −5.33E−06 | 0 | 0 | 0.002763508 | 0.000684486 | −7.57E−05 | 0.001066646 |
| 81909 | Zfpl1 | 0.000650312 | 0 | 0 | 0.002171201 | 0.000257645 | 0.00026397 | 0.000113055 |
| 620078 | C130026I21Rik | 0 | 0 | 0 | 0.00080831 | 0.000850279 | 0.000694805 | 0 |
| 21898 | Tlr4 | 0.000538872 | 0 | 0 | 0.002110354 | 0.00083678 | 0.000214585 | 0.001018243 |
| 19179 | Psmc1 | −0.00030054 | 0 | 0 | 0.002124283 | 0.000595602 | −0.00030324 | 0.000615792 |
| 52679 | E2f7 | 0.00156273 | 0 | 0 | 0.003044315 | 0.000933523 | 0.001416598 | 0.000718863 |
| 16509 | Kcne1 | −0.00053549 | 0 | 0 | 0 | −0.0010584 | 0.000137431 | 0 |
| 15516 | Hsp90ab1 | −0.00038386 | 0 | 0 | −0.00206564 | −0.00064914 | −0.0007356 | −0.00112164 |
| 320183 | Msrb3 | 0.000325349 | 0 | 0 | 0.001842085 | 0.001145988 | 0.000887429 | 0.001317663 |
| 217826 | Kcnk13 | 0.000393947 | 0 | 0 | −0.0014716 | −0.00078588 | −0.00065672 | −0.00081539 |
| 69068 | Tcim | −0.00019886 | 0 | 0 | −0.00185487 | −0.00059412 | −0.00089903 | −0.00150519 |
| 22701 | Zfp41 | −0.00125428 | 0 | 0 | −0.00318795 | −0.00154352 | −0.00118757 | −0.00195892 |
| 12580 | Cdkn2c | −0.00113864 | 0 | 0 | 0.002003998 | −2.41E−06 | −0.00018849 | 0.001846344 |
| 237222 | Ofd1 | −0.00027514 | 0 | 0 | 0.000620893 | 0.0004913 | 0.000573077 | 0.001034997 |
| 18946 | Pnliprp1 | −0.000423 | 0 | 0 | 0 | −0.00021279 | −0.00106928 | 0 |
| 17068 | Ly6d | −0.00189385 | 0 | 0 | −0.00226294 | −0.00111706 | −0.00055671 | 0.001184763 |
| 93879 | Pcdhb8 | 0.000335834 | 0 | 0 | 0 | 0.00041501 | 0.000361309 | 0 |
| 19354 | Rac2 | −0.00076634 | 0 | 0 | −0.00195042 | 0.000326082 | −0.00024736 | −0.00081831 |
| 19220 | Ptgfr | 6.04E−05 | 0 | 0 | 0.00210152 | 0.00058416 | 0.000187653 | 0 |
| 67371 | Gtf3c6 | 0.000329772 | 0 | 0 | −0.00243987 | −0.00020238 | 0.00036147 | 0.000267049 |
| 72141 | Adpgk | −0.00091388 | 0 | 0 | −0.0032515 | −0.0006908 | −0.00055568 | −0.00111478 |
| 231207 | Cpeb2 | −0.00022771 | 0 | 0 | 0.002758205 | 0.000953305 | −0.00023337 | 0.001286968 |
| 78913 | Ltn1 | −0.00121593 | 0 | 0 | −0.00341442 | −0.00087885 | −0.00100375 | −0.00095698 |
| 13447 | Doc2b | 0.00032585 | 0 | 0 | 0.002614021 | 0.000874072 | 0.000427506 | 0 |
| 12368 | Casp6 | 0.000386664 | 0 | 0 | −0.00144617 | −0.00034115 | 0.000747446 | −0.00030373 |
| 71326 | Treml1 | 0 | 0 | 0 | −0.00219295 | −0.00090988 | 0 | 0 |
| 237553 | Trhde | 0.000938708 | 0 | 0 | 0.001924821 | 0.000306133 | 0.00104106 | 0.000309896 |
| 69149 | Kbtbd3 | 0.000286717 | 0 | 0 | −0.00154111 | −0.00037566 | −0.0003622 | −0.00111891 |
| 17907 | Mylpf | −0.0004303 | 0 | 0 | −0.00042938 | −0.00054483 | 7.37E−05 | 0.000947096 |
| 84004 | Mcam | −0.00038093 | 0 | 0 | 0.002455558 | 0.001246012 | 0.000515917 | 0.001157654 |
| 114642 | Brdt | −0.00095198 | 0 | 0 | −0.00217404 | −0.0010696 | −0.00018343 | −0.00104922 |
| 23853 | Def6 | 0.000752838 | 0 | 0 | −0.00339389 | −0.00120029 | 0.000636839 | −0.00142402 |
| 57385 | P2ry4 | −0.00052022 | 0 | 0 | −0.00057667 | 9.15E−05 | −0.00047008 | 0 |
| 14961 | H2-Ab1 | 0.00109388 | 0 | 0 | −0.00112181 | 0.001611892 | −0.00091945 | −0.0004037 |
| 23917 | Impdh1 | −0.00081499 | 0 | 0 | −0.00158547 | −0.00055628 | −0.00061965 | −0.00049414 |
| 545486 | Tubb1 | 2.85E−05 | 0 | 0 | 0.002786272 | 0.001302559 | 0.000525785 | 0 |
| 68695 | Hddc3 | −0.00025017 | 0 | 0 | −0.00061474 | 0.000670003 | −0.00029265 | 2.07E−05 |
| 140742 | Sesn1 | 0.000558268 | 0 | 0 | −0.00278305 | −0.00138646 | 0.001046947 | −0.00090639 |
| 12854 | Cort | −0.00050219 | 0 | 0 | 0 | 0 | −0.00030276 | 0 |
| 216551 | Lgalsl | −0.00063347 | 0 | 0 | −0.00265505 | −0.00017548 | −0.00034262 | −0.00025889 |
| 21411 | Tcf20 | −0.00128121 | 0 | 0 | −0.00322106 | −0.00122312 | −0.001039 | −0.00179617 |
| 242594 | Fyb2 | −0.00145753 | 0 | 0 | −0.00344894 | −0.00088586 | −0.00111778 | 0 |
| 18514 | Pbx1 | 0.000232097 | 0 | 0 | 0.003105282 | 0.000616263 | 0.000292589 | 0.001985416 |
| 321019 | Gpr183 | 0 | 0 | 0 | 0.003036694 | 0.000855091 | 0 | 0.000907892 |
| 20378 | Frzb | 0.000852522 | 0 | 0 | 0.002294971 | 0.000268422 | 0.000270219 | 0.001169881 |
| 230484 | Usp1 | −0.00058818 | 0 | 0 | 0.001189663 | 0.000211409 | −8.04E−05 | 0.000354285 |
| 56213 | Htra1 | −0.00059538 | 0 | 0 | −0.00255267 | −0.00101767 | −0.00091483 | −0.00156058 |
| 240023 | Pnldc1 | 0.001106321 | 0 | 0 | 0.002761706 | 0.00042482 | 0.000310917 | 0 |
| 171180 | Syt12 | −0.00145121 | 0 | 0 | −0.00318855 | −0.00091534 | −0.00127617 | −0.00190909 |
| 54672 | Adgrg3 | −0.00128976 | 0 | 0 | −0.00182563 | −0.00031985 | −0.00095978 | −0.00066138 |
| 14255 | Flt3 | 0.000137709 | 0 | 0 | −0.00254352 | −0.00059258 | −0.00067816 | 0 |
| 327826 | Frs2 | −0.00028482 | 0 | 0 | −0.00192571 | −0.0003277 | −7.77E−05 | −0.00064511 |
| 20649 | Sntb1 | −3.44E−06 | 0 | 0 | −0.00153185 | −0.00100823 | 0.000499251 | −0.00101063 |
| 76464 | Kni1 | 0 | 0 | 0 | 0.003367295 | 0.001335353 | 0 | 0 |
| 70945 | Mmrn1 | −0.00149553 | 0 | 0 | −0.00261338 | −0.00028716 | 0 | −7.11E−05 |
| 83962 | Btbd1 | 0.000516129 | 0 | 0 | 0.002824529 | 0.001182276 | 0.000264809 | 0.001783489 |
| 259300 | Ehd2 | −0.0008316 | 0 | 0 | −0.0006803 | −0.00014314 | −0.00125755 | −0.00076331 |
| 231287 | Atp10d | −0.00100151 | 0 | 0 | −0.00299318 | −0.00125573 | −0.00080345 | −0.0011753 |
| 76566 | Rfinb | −0.00092878 | 0 | 0 | −0.00143798 | −0.00083998 | −0.00037201 | −0.00018111 |
| 240752 | Pik3c2b | 0.000609128 | 0 | 0 | 0.003137116 | 0.000911403 | 0.000591729 | 0.001259723 |
| 112422 | Zfp979 | 0.001831085 | 0 | 0 | 0.002512576 | 0.000788851 | 0.001360284 | 0 |
| 268480 | Rapgefl1 | −4.72E−05 | 0 | 0 | −0.00290394 | −0.00168689 | 2.15E−06 | −0.00128501 |
| 57430 | Sult3a1 | −0.00114166 | 0 | 0 | 0 | −0.00164141 | −0.0017467 | 0 |
| 226153 | Twnk | 0.000114387 | 0 | 0 | −0.00222046 | −0.0007556 | −0.00057891 | −0.00104817 |
| 69836 | Pla2g12b | 0.000644809 | 0 | 0 | 0.002995091 | 0.00112023 | 0.001087971 | 0 |
| 110935 | Atp6v1b1 | −0.00050095 | 0 | 0 | 0 | −0.00062588 | −0.00026008 | 0 |
| 73666 | Thoc3 | 0.000449382 | 0 | 0 | 0.003013441 | 0.001039745 | 0.000421002 | 0.001549728 |
| 218460 | Wdr41 | −3.02E−05 | 0 | 0 | −0.002138 | −0.00078007 | 0.000367213 | −0.00063543 |
| 269152 | Kif26b | −0.00080214 | 0 | 0 | 0.001964184 | 0.000480907 | −0.00078866 | 0.000300709 |
| 14680 | Gnal | −0.00044547 | 0 | 0 | 0.002506753 | 0.000306962 | −0.00018775 | 0.001164611 |
| 14763 | Gpr37 | 0.000935517 | 0 | 0 | 0.001940405 | 0.000137996 | 0.000275393 | 0 |
| 21412 | Tcf21 | 0.000329036 | 0 | 0 | 7.98E−06 | −0.00046212 | 0.000268519 | −0.00173029 |
| 16779 | Lamb2 | −0.00108499 | 0 | 0 | −0.00149838 | −0.00078833 | −0.00132345 | −0.00094254 |
| 21825 | Thbs1 | −0.00170287 | 0 | 0 | 0.001389845 | 2.98E−05 | −0.00117104 | 0.000555874 |
| 67629 | Spc24 | −0.00113002 | 0 | 0 | −0.00224381 | −0.00070149 | −0.00089317 | −0.00142588 |
| 13345 | Twist2 | 0 | 0 | 0 | 0.00221938 | 0.000765087 | 0 | 0 |
| 26944 | Tinag | −0.00087454 | 0 | 0 | 0 | −0.00060432 | −0.00102892 | 0 |
| 13035 | Ctsg | 0.000356146 | 0 | 0 | 0.001944273 | 0.00055274 | 0.001750036 | 0 |
| 621080 | Al429214 | −0.00034684 | 0 | 0 | 0 | 0 | −0.00094088 | 0 |
| 16456 | F11r | 0.000713629 | 0 | 0 | 0.00317098 | 0.000482073 | 0.001012287 | 0.000810152 |
| 14187 | Akr1b8 | 0.001120906 | 0 | 0 | 0.001564702 | 0.000615674 | 0.001123148 | 0.000961094 |
| 67216 | Mboat2 | −0.00114596 | 0 | 0 | −0.00277032 | −0.00157331 | −0.00082442 | −0.0019275 |
| 271564 | Vps13a | 0.00029287 | 0 | 0 | 0.002881939 | 0.001313958 | 0.000632585 | 0.001722868 |
| 14190 | Fgl2 | 0.000499472 | 0 | 0 | −0.00093227 | −0.000206141 | −6.21E−05 | −0.00053188 |
| 13371 | Dio2 | 0.000251298 | 0 | 0 | −0.00209385 | −0.00095186 | −0.00044178 | −0.00026917 |
| 21390 | Tbxa2r | 0.000591146 | 0 | 0 | −0.0020345 | −0.00121726 | 0.000191416 | −0.00169178 |
| 72789 | Veph1 | 0.000917095 | 0 | 0 | −0.0005713 | 0.000635503 | 0.001033509 | 0 |
| 76976 | Arxes2 | −0.00020545 | 0 | 0 | −0.00326409 | −0.0006175 | −0.00028295 | 0 |
| 27883 | Tango2 | 0.000565517 | 0 | 0 | −0.00152228 | −0.00066157 | −0.00016008 | −0.00106781 |
| 667370 | Ifit3b | 0.000685101 | 0 | 0 | 0.003320117 | 0.000967795 | 9.70E−05 | 0 |
| 16534 | Kcnn4 | −0.00020775 | 0 | 0 | −0.00234556 | 0.00011318 | 0.000367186 | 0.000108042 |
| 68490 | Zfp579 | −0.00034657 | 0 | 0 | 0.001872098 | 0.000567472 | −4.71E−05 | 0.000669846 |
| 67445 | C1qtnf4 | −0.00029956 | 0 | 0 | 0.001703357 | 0.001019439 | −8.03E−05 | 0.001735194 |
| 22151 | Tubb2a | −0.0008692 | 0 | 0 | −0.00078418 | −0.00096407 | −0.00049978 | −0.00079155 |
| 15376 | Foxa2 | 0.00049656 | 0 | 0 | −0.00110225 | 3.68E−05 | −0.00011651 | −0.00033575 |
| 27384 | Akr1c13 | −0.00033995 | 0 | 0 | 0.002262543 | 0.000922971 | −0.00103468 | 0.000349998 |
| 210503 | Zfp677 | 0 | 0 | 0 | 0.002305407 | 0.000828562 | 0 | 0 |
| 12458 | Ccr6 | −0.00064254 | 0 | 0 | −0.00172873 | −0.00048998 | −0.00016697 | 0 |
| 83814 | Nedd4l | −0.00042305 | 0 | 0 | −0.00223964 | 0.000157457 | 0.000538042 | 0.000997089 |
| 271786 | Galnt13 | 0 | 0 | 0 | −0.0001238 | 0.000154883 | 0 | 0 |
| 64085 | Clstn2 | 0.001225206 | 0 | 0 | 0.000507289 | 0.000532388 | 0.000381256 | −0.00030621 |
| 14134 | Fcnb | 0.001067101 | 0 | 0 | 0 | 0.000174166 | 0 | 0 |
| 110902 | Chrna2 | −0.00126403 | 0 | 0 | −0.00101933 | −0.0006495 | −0.00075378 | −.0003063 |
| 243912 | Hspb6 | −0.00139359 | 0 | 0 | −0.00078307 | −0.00031822 | −0.00112027 | −0.000463 |
| 105298 | Epdr1 | −0.00042924 | 0 | 0 | −0.00161177 | −2.70E−05 | −0.00109441 | −0.00022643 |
| 67389 | C1qtnf12 | −0.00025688 | 0 | 0 | 0.002388838 | 0.000738378 | 4.66E−05 | 0.00102666 |
| 14938 | Gzma | −0.00061928 | 0 | 0 | 0 | −0.00082735 | 0.000105811 | 0 |
| 11535 | Adm | 0.001722688 | 0 | 0 | 0.001593218 | 0.000897431 | 0.001612557 | 0.001200386 |
| 14394 | Gabra1 | 0.000499591 | 0 | 0 | 0.000240285 | 8.26E−05 | 0.000193102 | 0 |
| 229003 | Helz2 | 0.00037515 | 0 | 0 | −0.00063389 | −0.00016321 | 0.000856484 | 0.000773446 |
| 228775 | Trib3 | −0.0006005 | 0 | 0 | −0.00015986 | −0.00066995 | −0.00157819 | −0.00094556 |
| 20295 | Ccl17 | −2.08E−05 | 0 | 0 | 0 | −0.00047373 | −3.19E−05 | 0 |
| 192113 | Atp12a | 0.000180937 | 0 | 0 | 0 | −0.00080749 | −0.00010654 | 0 |
| 12500 | Cd3d | 0.000205131 | 0 | 0 | 0.0031546 | 0.00056497 | 0.000212299 | 0 |
| 52184 | Odf2l | 0.000665356 | 0 | 0 | 0.002350517 | 0.000742692 | 0.000937279 | 0.00142162 |
| 13608 | Edar | −0.00059748 | 0 | 0 | 0 | −0.00036801 | −0.00029154 | 0 |
| 94279 | Sfxn2 | 8.77E−05 | 0 | 0 | 3.53E−05 | −0.0003288 | −1.32E−07 | 0.000697152 |
| 20964 | Syn1 | −0.00013078 | 0 | 0 | 0.000278795 | 0.000149219 | 0 | 0 |
| 18619 | Penk | 0 | 0 | 0 | −0.00286069 | 6.20E−05 | 0 | −0.00077776 |
| 207911 | Mchr1 | 0 | 0 | 0 | −0.00098945 | −6.61E−05 | 0 | 0 |
| 109594 | Lmo1 | −0.00033311 | 0 | 0 | −0.00243906 | −0.00080645 | −0.00085461 | −0.00159747 |
| 68498 | Tspan11 | 0.000148566 | 0 | 0 | −0.00118827 | −0.00107426 | −0.00032955 | −0.00152808 |
| 70377 | Derl3 | −0.00096112 | 0 | 0 | 0.000693183 | 0.000988886 | 0.000488583 | 0.001576651 |
| 98363 | Efhd1 | −0.00010135 | 0 | 0 | 0.001734145 | 0.000591563 | −0.00035007 | 0.00127146 |
| 12023 | Barx2 | −1.96E−05 | 0 | 0 | 0.003243427 | 0.001030661 | −3.49E−05 | 0 |
| 170752 | Bco2 | 0.000483943 | 0 | 0 | 0.001527264 | 0.001282257 | 0.000469417 | 0.001805826 |
| 329366 | Ccdc187 | −0.00043468 | 0 | 0 | −0.00158056 | −0.00091828 | −0.00039986 | 0 |
| 14696 | Gnb4 | 0.000402269 | 0 | 0 | −0.00107353 | −0.00129518 | 5.55E−05 | −0.00102382 |
| 72169 | Trim29 | −0.00067113 | 0 | 0 | −0.00025585 | −0.00056616 | −0.00010343 | 0 |
| 73804 | Klf2c | −0.0001614 | 0 | 0 | 0.002422634 | 0.000616837 | −0.0004781 | 0 |
| 20410 | Sorbs3 | −5.92E−05 | 0 | 0 | 0.001083852 | 0.000565723 | 0.000916119 | 0.00146564 |
| 78284 | Creb3l4 | 0.000460567 | 0 | 0 | −0.00191706 | −0.0013616 | 0.000127194 | 0 |
| 17130 | Smad6 | 0.001720425 | 0 | 0 | 0.001226553 | 0.001074395 | 0.001437167 | 0.000997704 |
| 58222 | Rab37 | 1.59E−06 | 0 | 0 | 0.00024515 | 0.000461512 | −0.00052363 | 0.000156866 |
| 17474 | Clec4d | 0.000486821 | 0 | 0 | 0 | 0.001119555 | 0.000706889 | 0 |
| 16783 | Lamp1 | 0.000283473 | 0 | 0 | 0.002731042 | 0.000780233 | 0.000456381 | 0.001373835 |
| 18439 | P2rx7 | 0.000726738 | 0 | 0 | 0.002874489 | 0.000648751 | 0.000506982 | 0.000675457 |
| 18969 | Pola2 | −0.00090243 | 0 | 0 | −0.00056009 | −0.00077783 | −0.00106437 | −0.00054787 |
| 243374 | Gimap8 | 0.000878148 | 0 | 0 | 7.59E−05 | −0.00021881 | 0.000372654 | −0.00033261 |
| 11576 | Afp | 0.001124289 | 0 | 0 | 0.001190771 | 0.00049169 | 3.99E−05 | 0.00030609 |
| 16002 | Igf2 | −0.0001678 | 0 | 0 | 0.001877191 | 0.000565379 | −4.02E−05 | 0 |
| 14129 | Fcgr1 | 2.00E−05 | 0 | 0 | 0.002346593 | 0.001384221 | −8.77E−05 | 0.001111805 |
| 75590 | Dusp9 | −0.00043527 | 0 | 0 | −0.00324168 | −0.00085697 | −0.00035092 | 0 |
| 381319 | Batf3 | 0.000272414 | 0 | 0 | −0.00123844 | −8.58E−05 | −0.00023893 | 0 |
| 14635 | Galk1 | −0.00097368 | 0 | 0 | −0.00287908 | −0.00067272 | −0.00120257 | −0.00164859 |
| 11720 | Mat1a | 0.001119747 | 0 | 0 | −0.00108386 | −0.00111496 | 0.00179478 | 0 |
| 108897 | Aif1l | −4.15E−05 | 0 | 0 | 0.001723016 | 0.001597641 | −0.0002763 | 0.001622887 |
| 210673 | Prrt3 | −0.00013424 | 0 | 0 | 0.001650242 | 0.000393306 | −0.00017835 | 0 |
| 72434 | Lypd3 | −0.00089437 | 0 | 0 | 0 | −0.00080807 | −0.00088331 | 0 |
| 238988 | Erc2 | −0.00087345 | 0 | 0 | 0.002490744 | 0.001043448 | −0.00047159 | 0.002104637 |
| 14205 | Vegfd | −5.88E−05 | 0 | 0 | 0.001789407 | 0.001052053 | 0.00047706 | 0.000472007 |
| 384009 | Glipr2 | −0.00079165 | 0 | 0 | −1.15E−06 | 0.000625901 | −0.0005762 | 0.000584962 |
| 73720 | Cst6 | −0.00038744 | 0 | 0 | 0.000316914 | 0.000627005 | −0.00067238 | 0 |
| 20191 | Ryr2 | 0.000327897 | 0 | 0 | 0.001842332 | 0.000307506 | 0.000468133 | 0.000394041 |
| 16775 | Lama4 | −0.00161697 | 0 | 0 | −0.00202825 | −0.00135546 | −0.00146185 | −0.00193346 |
| 67486 | Polr3g | 0.001595187 | 0 | 0 | 0.002856105 | 0.001213215 | 0.001140814 | 0.000725153 |
| 66431 | Oxld1 | 0.000186301 | 0 | 0 | 0.000666821 | 0.000183474 | 0.000454416 | 0.000322146 |
| 243914 | Lgi4 | 0.000335657 | 0 | 0 | −0.00140848 | −0.00106801 | −0.00067883 | −0.00158436 |
| 76187 | Adhfe1 | −0.00066559 | 0 | 0 | −0.00304743 | −0.00122403 | −0.00098823 | −0.00160634 |
| 74195 | Elp3 | 0.000118201 | 0 | 0 | 0.003039605 | 0.001019586 | 0.000133125 | 0.001658348 |
| 232801 | Lilra5 | −0.00073462 | 0 | 0 | 0 | −0.00010325 | −0.00102251 | 0 |
| 226025 | Trpm3 | −0.00063898 | 0 | 0 | −0.00197247 | −0.000523 | −0.00063562 | −0.00147765 |
| 217125 | Samd14 | 0.000354488 | 0 | 0 | 0.002366304 | 0.001204627 | 0.000187539 | 0.000724782 |
| 16402 | Itga5 | 0.00034044 | 0 | 0 | 0.002836314 | 0.000993322 | 0.000587915 | 0.001775885 |
| 14809 | Grik5 | 2.01E−05 | 0 | 0 | −0.00137268 | −0.0003301 | 0.00034315 | 0.000203062 |
| 18019 | Nfatc2 | −0.00021013 | 0 | 0 | −0.00178635 | −0.00014092 | −0.00142833 | −0.00133289 |
| 214685 | Chadl | 0 | 0 | 0 | 0.002286203 | 0.00107198 | 0 | 0 |
| 11606 | Agt | 0.000741114 | 0 | 0 | 0.00071948 | −0.00103199 | 0.000183149 | −0.00117856 |
| 18034 | Nfkb2 | 0.000250728 | 0 | 0 | −1.11E−05 | 0.000464971 | 0.000464839 | 0.000278483 |
| 224674 | Slc37a1 | −0.00097468 | 0 | 0 | −0.00240477 | −0.0012678 | −0.00169201 | −0.00135331 |
| 75538 | Gerin5a | 0.000137402 | 0 | 0 | −0.00312154 | −0.00032833 | 0.000581484 | 0 |
| 72415 | Sgo1 | −8.98E−05 | 0 | 0 | −0.0005088 | 0.000157387 | 0.000409922 | 0 |
| 665270 | Plb1 | 6.17E−05 | 0 | 0 | 0.000602834 | −0.00032117 | 9.72E−05 | −0.00045576 |
| 94332 | Cadm3 | −0.00119019 | 0 | 0 | −0.00126223 | 0.000449347 | −0.00095259 | 0.000702099 |
| 68612 | Ube2c | −0.00040663 | 0 | 0 | −0.00341289 | −0.00158962 | 7.97E−05 | −0.00137017 |
| 14401 | Gabrb2 | 0 | 0 | 0 | −0.00108066 | −0.00041534 | 0 | 0 |
| 14673 | Gna12 | 0.000434668 | 0 | 0 | 0.000217268 | 0.000162715 | 0.000541436 | −0.00049292 |
| 11504 | Adamts1 | −0.00026727 | 0 | 0 | 0.000152944 | 0.00097397 | 0.000626387 | 0.000546237 |
| 71756 | Cpn2 | 0.000621444 | 0 | 0 | −0.00056533 | −0.00039139 | 0.000481624 | 0.000537769 |
| 19885 | Rorc | −0.00053627 | 0 | 0 | 0.002152756 | 0.000750215 | −0.00105746 | 0.001343769 |
| 19240 | Tmsb10 | 0.000122577 | 0 | 0 | −0.00124033 | −8.14E−05 | −0.00042916 | −6.45E−05 |
| 54396 | Irgm2 | −0.00102347 | 0 | 0 | −0.00216068 | −0.0001187 | 0.000280138 | 0.000240834 |
| 70337 | Iyd | 0.00083869 | 0 | 0 | 0.00198042 | 0.001256739 | 0.000848168 | 0.001586866 |
| 18476 | Pafah1b3 | −0.00015669 | 0 | 0 | 0.001232107 | 0.000780715 | 0.000314625 | 0.001203947 |
| 214058 | Megf11 | −0.00100416 | 0 | 0 | −0.00079952 | −0.00017313 | −0.00020269 | 0.000492272 |
| 67928 | Abca14 | 0.000168815 | 0 | 0 | −0.00087694 | −0.00057033 | 0.000194165 | 0 |
| 110521 | Hivep1 | −0.00020868 | 0 | 0 | −0.00206071 | −0.00075597 | −1.02E−05 | −0.00105758 |
| 11790 | Speg | −0.00069195 | 0 | 0 | 0.001707872 | 0.000566698 | −0.00104795 | 0.000424073 |
| 68854 | Asb11 | −0.00080609 | 0 | 0 | 7.02E−05 | 0.000366065 | −0.0005307 | 0 |
| 171284 | Timd2 | −0.0001999 | 0 | 0 | 0.000358455 | 0.000190902 | −0.00029458 | 0 |
| 17089 | Lyar | −0.00095864 | 0 | 0 | −0.00266181 | −0.00124366 | −0.00132163 | −0.00169055 |
| 14615 | Gjc1 | −2.63E−05 | 0 | 0 | −0.00081195 | −0.00064087 | −0.00045835 | −0.00029238 |
| 13417 | Dnah8 | −0.00065093 | 0 | 0 | −0.00250215 | −0.00071351 | 0.000755047 | 0.000357291 |
| 71233 | Enkur | 0 | 0 | 0 | −2.50E−05 | 0.000530847 | 0 | 0 |
| 238076 | Kcns3 | −0.00064776 | 0 | 0 | −0.00128619 | −0.00017029 | −0.0004801 | −0.00045672 |
| 52202 | Rbm34 | −0.00011064 | 0 | 0 | 0.003085703 | 0.0011922 | 0.000319607 | 0.001634105 |
| 13821 | Epb41l1 | −0.00021819 | 0 | 0 | −0.00033174 | −0.0003185 | −0.00077996 | 0.000457704 |
| 15483 | Hsd11b1 | −0.00037444 | 0 | 0 | −0.00023168 | 4.07E−05 | −5.22E−06 | −0.00034593 |
| 22786 | Zp1 | 0 | 0 | 0 | 0 | 0 | 0 | 0 |
| 545192 | Baiap3 | 0 | 0 | 0 | 0.002875141 | 0.001232085 | 0 | 0 |
| 16669 | Krt19 | −9.87E−05 | 0 | 0 | −0.0018573 | −0.00171595 | 0.0002405 | −0.00200712 |
| 75645 | Ccdc172 | 0 | 0 | 0 | 0 | 0 | 0 | 0 |
| 54561 | Nap1l3 | 0.000414785 | 0 | 0 | 0.001241016 | 0.001052858 | 0.001212427 | 0.001746875 |
| 106878 | Smim3 | −0.00016029 | 0 | 0 | 1.85E−05 | −0.00063479 | −3.11E−05 | −0.00127995 |
| 19073 | Srgn | 0.000350615 | 0 | 0 | −0.00047627 | −0.00036936 | 0.0008523 | 0.00010217 |
| 72014 | Btbd17 | 0.000156975 | 0 | 0 | −0.00045019 | −0.00035155 | 0.000245629 | 0 |
| 110446 | Acat1 | 0.000373372 | 0 | 0 | 0.002909651 | 0.000946193 | −0.00034003 | 0.001362208 |
| 18548 | Pcsk1 | 0 | 0 | 0 | 0.000917021 | 0.000661357 | 0 | 0 |
| 226830 | Smyd2 | −0.0017023 | 0 | 0 | 0.000420692 | 0.00073784 | −0.00107874 | 0.000496142 |
| 100038882 | Isg15 | 0.001401077 | 0 | 0 | 0.001024817 | 0.000497597 | 0.000730386 | 0.000960156 |
| 260301 | Otos | 0 | 0 | 0 | 0 | 0 | 0 | 0 |
| 11829 | Aqp4 | 0.001677208 | 0 | 0 | 0.002595778 | 0.001358015 | 0.001108254 | 0.001498779 |
| 68236 | Gtsf1l | 0.000410879 | 0 | 0 | 0 | 0 | 0.000613598 | 0 |
| 16170 | Il16 | 0.000126874 | 0 | 0 | −0.00223492 | −0.00132546 | 0.000387927 | −0.00199875 |
| 17463 | Psmd7 | 0.000213768 | 0 | 0 | −0.00156839 | −0.00011124 | 0.000650171 | −7.12E−05 |
| 12700 | Cish | 0.000838972 | 0 | 0 | −0.00319718 | −0.00105681 | 9.80E−05 | −0.00123744 |
| 272428 | Acsm5 | 0.001167702 | 0 | 0 | −0.00150535 | 0.000838294 | 0.000503606 | −0.00211945 |
| 17380 | Mme | 0.000148787 | 0 | 0 | −0.0023228 | −0.000235 | 0.000653317 | −0.0002119 |
| 225997 | Trpm6 | 0.000348334 | 0 | 0 | −0.00318148 | −0.00045589 | 0.000380266 | −0.00084696 |
| 12579 | Cdkn2b | 0.001641952 | 0 | 0 | 0.002594668 | 0.001181871 | 0.001153953 | 0.000865391 |
| 77113 | Klhl2 | −0.00057788 | 0 | 0 | −0.00219268 | −0.00096012 | −0.00046319 | −0.00076184 |
| 102774 | Bbs4 | 0.000996191 | 0 | 0 | 0.003032166 | 0.000993457 | 0.001097383 | 0.001662809 |
| 20888 | Sult1c1 | 0.000850089 | 0 | 0 | 0 | 0 | 0.000746621 | 0 |
| 16516 | Kcnj15 | 9.29E−05 | 0 | 0 | 0.002038256 | 0.001527069 | 0.00062965 | 0.001810192 |
| 19085 | Prkar1b | 0.001504123 | 0 | 0 | 0.002427222 | 0.001187106 | −0.00025181 | −0.00033359 |
| 13385 | Dlg4 | 0.001411646 | 0 | 0 | −0.00104481 | −0.00097673 | 0.000132579 | −0.00143961 |
| 225845 | Plaat3 | −0.0011085 | 0 | 0 | −0.00085053 | 0.00022045 | −0.00081874 | 0 |
| 14842 | Gsx1 | 0 | 0 | 0 | 0 | 0 | 0 | 0 |
| 17433 | Mobp | −0.00016402 | 0 | 0 | −0.0013702 | −0.00061972 | −9.75E−05 | 0 |
| 67623 | Tm7sf3 | 0.000363805 | 0 | 0 | 0.001451972 | 0.000922827 | 0.000367109 | 0.000804735 |
| 80286 | Tusc3 | −0.00028054 | 0 | 0 | −0.00235243 | −0.00128216 | −0.00036836 | −0.00147397 |
| 69564 | Nmrk2 | 0 | 0 | 0 | 0.002473178 | 0.000541444 | 0 | 0 |
| 20462 | Tra2b | 0.000508735 | 0 | 0 | 0.001241593 | 0.000932356 | 0.00078342 | 0.001120976 |
| 225579 | Slc27a6 | 0 | 0 | 0 | 0 | 0.001115615 | 0 | 0 |
| 20265 | Scn1a | 0 | 0 | 0 | −0.00010359 | 0.000300129 | 0 | 0 |
| 67245 | Peli1 | −4.70E−05 | 0 | 0 | −0.00192153 | −0.00105887 | −0.00037924 | −0.00154947 |
| 13479 | Dpep1 | −0.00161491 | 0 | 0 | 0.001157479 | 5.08E−05 | −0.00176851 | −0.00079078 |
| 21817 | Tgm2 | 0.000882216 | 0 | 0 | 0.002335876 | 0.00125269 | 0.000489497 | 0.00159656 |
| 26372 | Clcn6 | 0.001862035 | 0 | 0 | 0.002103179 | 0.001301825 | 0.001439031 | 0.001285458 |
| 56534 | Hspb3 | 0.000148696 | 0 | 0 | 0.001905629 | 0.001027089 | −0.00041064 | 0 |
| 74194 | Rnd3 | 0.000587483 | 0 | 0 | −0.00165576 | −0.00095569 | 2.46E−05 | −0.00073603 |
| 56448 | Cyp2d22 | −0.00192238 | 0 | 0 | −0.00140884 | −0.00094936 | −0.00172767 | −0.00076354 |
| 20230 | Satb1 | 0.001274374 | 0 | 0 | 0.000384457 | −0.00053898 | 0.000256102 | −0.00125267 |
| 75599 | Pcdh1 | 0.001124896 | 0 | 0 | 0.000914172 | 0.000899722 | 0.001251772 | 0.000532515 |
| 56421 | Pfkp | 0.000335912 | 0 | 0 | 0.002145955 | 0.000573831 | 0.000395586 | 0.000684106 |
| 105171 | Arrdc3 | 0.000967234 | 0 | 0 | −0.00285091 | −0.00047168 | 0.000244279 | −0.00140567 |
| 12322 | Camk2a | 0.000260491 | 0 | 0 | −0.00179418 | −0.00114563 | 0.000187217 | 2.10E−05 |
| 16504 | Kcnc3 | −0.00010749 | 0 | 0 | −0.00230928 | −0.00046563 | −0.00034507 | −0.00078684 |
| 232086 | Tmem150a | −0.001182 | 0 | 0 | −0.00223398 | −0.00067307 | −0.00072782 | −0.0011805 |
| 228852 | Ppp1r16b | −0.00118783 | 0 | 0 | −0.00340724 | −0.00147563 | −0.0011814 | −0.00187603 |
| 20199 | S100a5 | 0 | 0 | 0 | 0 | 0 | 0 | 0 |
| 110310 | Krt7 | −0.00126229 | 0 | 0 | −0.001754 | −0.00114997 | −0.00095167 | −0.00119888 |
| 630146 | Cd101 | 0 | 0 | 0 | 0 | 0.000571126 | 0 | 0 |
| 195209 | Zfp469 | 0 | 0 | 0 | 0 | 0 | 0 | 0 |
| 53602 | Hpcal1 | 0.000738989 | 0 | 0 | −0.00041675 | −0.00025984 | 0.000454506 | −0.00068025 |
| 108907 | Nusap1 | −0.00106061 | 0 | 0 | 0.000816755 | −0.00053136 | −0.00063074 | 7.22E−05 |
| 66454 | Nmnat1 | −0.00092005 | 0 | 0 | −0.00272246 | −0.0010286 | −0.00143503 | −0.00147962 |
| 382571 | Kcnf1 | 0 | 0 | 0 | −0.00215118 | −0.000936 | 0 | 0 |
| 17912 | Myo1b | −0.00092222 | 0 | 0 | −0.00308633 | −0.00145464 | −0.00091379 | −0.00184761 |
| 67856 | Echdc3 | −8.26E−05 | 0 | 0 | −0.00173435 | −0.00092168 | 0.000172944 | −0.00173806 |
| 14991 | H2-M3 | 0.000943972 | 0 | 0 | 0.002645253 | 0.001209826 | 8.80E−05 | −0.00012699 |
| 403187 | Opa3 | 0.000109629 | 0 | 0 | 0.003110844 | 0.000563518 | 5.55E−05 | 0.001099442 |
| 66106 | Smpx | 0.00013845 | 0 | 0 | 0.001662254 | 0.00080753 | 0.000543814 | 0 |
| 229600 | BC028528 | 0.000439827 | 0 | 0 | −0.00087038 | −0.00021222 | 0.000514921 | 0.000533211 |
| 23886 | Gdf15 | −0.00057297 | 0 | 0 | −0.00046727 | 0.00126083 | −0.00010988 | 0.001238476 |
| 13849 | Ephx1 | 0.000642236 | 0 | 0 | 0.000228522 | 0.001323706 | −0.00042434 | 2.19E−05 |
| 13036 | Ctsh | 0.001831343 | 0 | 0 | 0.001848814 | 0.000900032 | 0.001178531 | 0.001447761 |
| 21809 | Tgfb3 | −0.00045849 | 0 | 0 | −0.00033653 | −0.00022247 | 0.000798914 | 0.000716446 |
| 109272 | Mybpc1 | 0.000353559 | 0 | 0 | 0.001090886 | 0.001008593 | 0.000103165 | 0.000405063 |
| 15212 | Hexb | 0.001180699 | 0 | 0 | −1.70E−05 | 0.000452155 | 0.001315677 | 0.000978456 |
| 76580 | Mib2 | −0.00141779 | 0 | 0 | −0.00292693 | −0.00079515 | −0.00122555 | −0.00083123 |
| 100072 | Camta1 | 0.000440897 | 0 | 0 | 0.001068759 | 0.000835274 | 2.14E−05 | 0.001428333 |
| 67931 | Serpini2 | 0 | 0 | 0 | 0 | 0 | 0 | 0 |
| 12845 | Comp | 0.000400621 | 0 | 0 | 0.002156499 | 0.000796798 | 0.000466175 | 0 |
| 67724 | Pop1 | −0.00105065 | 0 | 0 | −0.0016861 | −0.00113801 | −0.00098775 | −0.0009963 |
| 22673 | Zfp185 | 0.000468904 | 0 | 0 | 0.001488227 | 0.000626651 | 0.00027182 | 0.000810401 |
| 77220 | Tmem200a | 0 | 0 | 0 | −0.00269064 | −0.00139249 | 0 | 0 |
| 27280 | Phlda3 | 0.000972811 | 0 | 0 | 0.00067485 | 0.000946283 | 0.000789698 | 0.000574867 |
| 79565 | Mettl27 | 0.000412674 | 0 | 0 | 0.001386861 | 0.000416888 | 0.000348727 | 4.26E−05 |
| 224860 | Plcl2 | 0.000481882 | 0 | 0 | −0.00255524 | −0.00084134 | 0.000412161 | −0.00055911 |
| 73737 | Bbin | 0.00066592 | 0 | 0 | −0.00336144 | −0.00121931 | −0.00031541 | −0.0010907 |
| 93888 | Pcdhb17 | −0.00011629 | 0 | 0 | 0.001920843 | 0.001016519 | 0.000714751 | 0.001333932 |
| 84111 | Gpr87 | −8.05E−05 | 0 | 0 | 0 | 0.000636552 | −0.0001599 | 0 |
| 110855 | Pde6c | −0.00089876 | 0 | 0 | 0 | 1.36E−05 | −0.00161765 | 0 |
| 15450 | Lipc | −0.00176182 | 0 | 0 | 0.000349129 | 0.000420106 | −0.00112278 | 3.52E−05 |
| 14308 | Fshb | 0.00066602 | 0 | 0 | 0 | 0 | 0.001064044 | 0 |
| 20530 | Slc31a2 | −0.00131194 | 0 | 0 | −0.00210619 | −0.00095859 | −0.00117115 | −0.0009667 |
| 19225 | Ptgs2 | 0 | 0 | 0 | −0.00155218 | −0.00025654 | 0 | 0 |
| 74190 | Exoc3l4 | −0.00141928 | 0 | 0 | 0.003173281 | 0.000986697 | −0.00165302 | 0.001127888 |
| 105387 | Akr1c14 | −0.00107948 | 0 | 0 | −0.00207457 | −0.00090715 | −0.00129838 | −0.00119589 |
| 14282 | Fosb | 0 | 0 | 0 | −0.00053878 | 1.23E−05 | 0 | 0 |
| 17219 | Mcm6 | 0.000438825 | 0 | 0 | 0.002740092 | 0.000364727 | 0.000308113 | 0.000445567 |
| 234214 | Sorbs2 | 0.000143298 | 0 | 0 | 0.002177653 | 0.000941277 | −5.23E−06 | 0.000945244 |
| 71878 | Fam83d | 0 | 0 | 0 | 0.00334985 | 0.000884484 | 0 | 0 |
| 243780 | Dennd11 | −0.00142257 | 0 | 0 | −0.00292622 | −0.0013487 | −0.00094714 | −0.00096121 |
| 56543 | Kcnd3 | 0.000113031 | 0 | 0 | 0.002897104 | 0.001369825 | −0.00011204 | 0 |
| 12495 | Entpd1 | −0.00125178 | 0 | 0 | −0.00260454 | −0.00072681 | −0.00076667 | −0.0011477 |
| 234728 | Cmtr2 | 0.000685715 | 0 | 0 | 0.000945306 | 0.000389562 | 0.000607701 | 0.000673756 |
| 17250 | Abcc1 | −0.0015029 | 0 | 0 | −0.00298759 | −0.00090532 | −0.00089046 | −0.00066298 |
| 207565 | Camkk2 | −0.00086846 | 0 | 0 | −0.00094338 | −0.0007607 | −0.00059153 | −0.00017661 |
| 72461 | Prcp | −0.00106359 | 0 | 0 | −0.0025358 | −0.00086524 | −0.00013958 | −0.00016788 |
| 56868 | Psg23 | −0.00088017 | 0 | 0 | 0 | 0 | −0.00081727 | 0 |
| 16597 | Klf12 | 0.000160975 | 0 | 0 | −0.0006953 | 0.000452982 | 0.000202393 | 0.000627918 |
| 16202 | Ilk | −0.00018163 | 0 | 0 | −0.00098278 | −0.00132818 | −0.00059435 | −0.00189022 |
| 11556 | Adrb3 | 0.000728568 | 0 | 0 | 0.000833032 | 0.000349829 | −0.00097397 | −0.0017058 |
| 227696 | Phyhd1 | −0.00029265 | 0 | 0 | −0.00023656 | −4.51E−05 | −0.00018104 | −0.00017536 |
| 67427 | Rps20 | 0.000716273 | 0 | 0 | 0.002258072 | 0.001188683 | 0.000900293 | 0.002074601 |
| 19946 | Rpl30 | 0.000542095 | 0 | 0 | 0.001204812 | 0.000598221 | −0.00070699 | 0.000766827 |
| 12971 | Crym | −0.00022455 | 0 | 0 | 0.002464462 | 0.000252517 | −0.00145019 | −0.00042476 |
| 67942 | Atp5g2 | 0.000418015 | 0 | 0 | −0.00069706 | −0.00044936 | −0.00116056 | −0.00212997 |
| 100273 | Osbpl9 | −0.00053695 | 0 | 0 | 0.000291276 | 1.54E−05 | −0.00024634 | 0.000466063 |
| 28084 | Vps25 | −0.00011864 | 0 | 0 | −0.00041896 | −0.00085926 | −0.00043893 | −0.00167355 |
| 13537 | Dusp2 | −0.00057952 | 0 | 0 | −0.0019499 | −0.00018093 | −0.00035762 | −0.00171888 |
| 18212 | Ntrk2 | 0.001266212 | 0 | 0 | 0.001284304 | 0.001255854 | 0.001397008 | 0.001593875 |
| 67760 | Slc38a2 | −0.00038302 | 0 | 0 | −7.60E−05 | −0.00077775 | −2.27E−05 | −0.00050686 |
| 14683 | Gnas | −6.11E−05 | 0 | 0 | 0.000977891 | 0.00026855 | −0.0005313 | −0.00027981 |
| 20515 | Slc20a1 | 0.000144775 | 0 | 0 | 0.000286559 | 0.000807395 | 0.001483741 | 0.000823111 |
| 14950 | H13 | −0.00024073 | 0 | 0 | −0.0006773 | −0.00010441 | −0.00019315 | 0.000123775 |
| 70466 | Ckap2l | 5.93E−05 | 0 | 0 | −0.00093488 | −0.00074216 | 0.000444173 | −0.00055765 |
| 227522 | Rpp38 | 0.00029536 | 0 | 0 | 0.00167364 | 0.00013517 | 0.000554812 | 0.000452881 |
| 68939 | Rasl11b | 0.000757413 | 0 | 0 | −0.00179983 | −0.00037329 | 0.000766354 | −0.00037497 |
| 100039028 | Mup11 | 0 | 0 | 0 | 0 | 0 | −0.00160102 | 0 |
| 22413 | Wnt2 | −0.00114275 | 0 | 0 | −0.00108394 | −0.00119583 | −0.00064627 | −0.00058583 |
| 72125 | Amer2 | 0.000183897 | 0 | 0 | 0.000237095 | 0.000177724 | −0.00016423 | 0 |
| 53419 | Corin | 0.001207753 | 0 | 0 | 0.000899905 | 0.001035184 | 0.000654908 | 0.000330891 |
| 16763 | Lad1 | −0.00080023 | 0 | 0 | −0.00070522 | −0.00096357 | −0.00064011 | −0.00022071 |
| 22234 | Ugcg | 0.001503431 | 0 | 0 | 0.002332797 | 0.000517818 | −5.39E−05 | −0.00050787 |
| 12014 | Bach2 | 1.72E−05 | 0 | 0 | 0.000178344 | −7.49E−05 | 0.001048523 | 0.00072002 |
| 17524 | Mpp1 | 0.000590157 | 0 | 0 | −0.00023601 | −0.00022329 | −0.00018516 | −0.00126986 |
| 14864 | Gstm3 | 0.001048654 | 0 | 0 | −0.00094273 | −0.00017022 | −0.00065905 | 0 |
| 217335 | Fbf1 | −0.00142473 | 0 | 0 | −0.00167798 | −0.00100285 | −0.00157701 | −0.00175815 |
| 625018 | C4a | 0.000774957 | 0 | 0 | 0 | 0 | −0.0005905 | 0 |
| 406220 | Krt77 | −0.00049293 | 0 | 0 | 0.001463872 | −0.00025354 | 7.81E−05 | 0 |
| 78252 | Nxpe2 | −0.00068841 | 0 | 0 | −0.00113946 | −0.00032305 | −0.00131591 | −0.00169586 |
| 12686 | Elovl3 | 0.000550294 | 0 | 0 | −0.00235286 | −0.00067039 | −0.00089305 | −0.00117409 |
| 20219 | Apcs | 6.71E−05 | 0 | 0 | −0.00067029 | 0.000455678 | 0.001362613 | 0 |
| 225994 | Nmwrk1 | −1.49E−05 | 0 | 0 | 0.001913165 | 0.000601296 | −0.00167952 | 3.18E−05 |
| 56338 | Txnip | −0.00013841 | 0 | 0 | 0.000353188 | 0.000890579 | −0.00065071 | 0.000239189 |
| 17064 | Cd93 | 0.000665021 | 0 | 0 | 0.001183136 | −0.00029691 | 0.000383965 | 0.000398605 |
| 240549 | Gm4952 | −0.00047001 | 0 | 0 | 0 | 0.0004272 | −0.00048728 | 0 |
| 71898 | Apol9b | 0.000979496 | 0 | 0 | 0 | 0 | 0.001539133 | 0 |
| 100041658 | Mup7 | 0 | 0 | 0 | 0 | 0 | 0.001688418 | 0 |
| 12144 | Blm | 0.000478703 | 0 | 0 | 0.000914468 | 2.10E−05 | 0.000345263 | 0.000823273 |
| 18024 | Nfe2l2 | 0.000947674 | 0 | 0 | 0.002205739 | 0.00139287 | −0.00022346 | 0.000812627 |
| 16592 | Fabp5 | 0.000501451 | 0 | 0 | 0.001855174 | 0.000305806 | −0.00099957 | −0.00141425 |
| 228413 | Prrg4 | −0.00043397 | 0 | 0 | 0.000595218 | 0.000570312 | 0.000428548 | 0 |
| 320782 | Tmem154 | 2.10E−05 | 0 | 0 | 0.001099881 | 0.001472203 | −0.00062977 | 0.001394972 |
| 72776 | Sass6 | −0.00055396 | 0 | 0 | −0.00091945 | −0.00096436 | −0.00078386 | −0.00118411 |
| 216227 | Slc1728 | 0.001230917 | 0 | 0 | 0 | 0 | −0.00096358 | 0 |
| 21807 | Tsc22d1 | 0.001871196 | 0 | 0 | 0.001795984 | 0.000862656 | 0.001336882 | 0.000130563 |
| 12577 | Cdkn1c | 0.001124195 | 0 | 0 | 0.001708469 | 0.000810961 | 2.21E−05 | 0.000817287 |
| 19200 | Pstpip1 | 0.000985039 | 0 | 0 | 0.00130023 | 0.000299439 | −0.00031239 | 0.000124007 |
| 71687 | Tmem25 | −0.00015761 | 0 | 0 | 0.003249181 | 0.000917649 | −0.00049008 | 0.000120707 |
| 239122 | Setdb2 | 0.000255569 | 0 | 0 | −0.00011611 | −0.00021138 | 0.001238535 | 0.000846437 |
| 72948 | Tppp | −5.96E−05 | 0 | 0 | −0.00051724 | −0.00065148 | 0.000517455 | −0.0001587 |
| 21676 | Tead1 | −0.00070455 | 0 | 0 | −0.00214796 | −0.00069947 | 0.000350168 | 0.000470954 |
| 245522 | Zc4h2 | −5.26E−05 | 0 | 0 | −0.00173917 | −0.00053589 | 0.0008187 | −0.00010711 |
| 379043 | Raet1e | 0 | 0 | 0 | 0 | 0 | 0.00038001 | 0 |
| 68481 | Mpzl1 | −0.00144414 | 0 | 0 | −0.00273176 | −0.00122449 | 4.90E−05 | −0.00043284 |
| 22414 | Wnt2b | 0.000456178 | 0 | 0 | −0.00105258 | −0.00037724 | 0.000212301 | −0.00063444 |
| 77037 | Mrap | −2.03E−05 | 0 | 0 | −7.45E−05 | −0.00052503 | −6.66E−05 | −0.00033328 |
| 117198 | Ivns1abp | −0.00062025 | 0 | 0 | −0.00081459 | −0.00085727 | −0.00118413 | −0.00169553 |
| 70122 | Mllt3 | 0.000112423 | 0 | 0 | 0.002534651 | 0.000925155 | 0.000355753 | 0.000821582 |
| 404195 | Cyp2c54 | −0.00035788 | 0 | 0 | 0 | 0 | −0.00062298 | 0 |
| 211924 | Dsg1c | 0.00135545 | 0 | 0 | 0 | 0 | 0.00115945 | 0 |
| 217843 | Unc79 | 0.00042043 | 0 | 0 | 0.000680493 | 0.000210092 | 0.000137586 | 0.000156152 |
| 223881 | Rnd1 | −0.00099382 | 0 | 0 | −0.00066057 | 0.000422038 | 0.000279843 | 0.000461657 |
| 74521 | Ppp4r4 | −0.00074384 | 0 | 0 | 6.70E−05 | 0.000316911 | 0.00035095 | 0.001368631 |
| 67887 | Saraf | −3.31E−05 | 0 | 0 | 0.001507357 | 0.000257983 | 0.000522901 | 0.000365864 |
| 67846 | Tmem39a | −0.00013353 | 0 | 0 | 0.000850892 | 0.000189883 | −0.00064352 | −0.00033897 |
| 53791 | TlrS | −0.00129907 | 0 | 0 | −0.00260962 | −0.00100457 | −0.0007861 | −0.0013483 |
| 639769 | Adh6-ps1 | −0.00052235 | 0 | 0 | 0 | 0 | −0.00059005 | 0 |
| 212518 | Sprn | −0.00038268 | 0 | 0 | 0.001716304 | 0.000492332 | −0.00015284 | 0 |
| 16415 | Itgb3 | −0.00134525 | 0 | 0 | 0.000884073 | 3.92E−05 | −0.00039491 | 0.001133855 |
| 12660 | Chka | −8.10E−05 | 0 | 0 | 0.000647393 | 0.000372393 | 0.001097315 | 0.001839477 |
| 68861 | Dipk2a | −0.00066884 | 0 | 0 | −0.00121879 | −0.00070376 | −0.0014393 | −0.00155743 |
| 434794 | Xlr4a | 0 | 0 | 0 | 0 | 0 | −0.00124926 | 0 |
| 21952 | Tnni1 | −0.00030533 | 0 | 0 | 0.00080033 | 0.000873194 | −0.00040286 | 0 |
| 236643 | Sytl5 | 0.000277579 | 0 | 0 | 0 | 0.000232764 | −0.00034413 | 0 |
| 52040 | Ppp1r10 | 0.000503267 | 0 | 0 | 0.001774624 | 5.81E−05 | −0.00087729 | −0.00114581 |
| 68955 | Srrm4 | −0.00040027 | 0 | 0 | 0.0012559 | 0.000319304 | −0.00082875 | 0.000154458 |
| 21824 | Thbd | 0.000670956 | 0 | 0 | −0.00074517 | −0.0001584 | 0.001086147 | 0.0007415 |
| 268741 | Tox4 | −0.00069131 | 0 | 0 | −0.0021858 | −0.00077812 | 0.00018186 | −0.00053151 |
| 109222 | Rarres1 | −0.00075032 | 0 | 0 | −0.00171545 | −0.00017163 | −0.0013074 | −0.00135397 |
| 100503019 | Gm16551 | −0.00054873 | 0 | 0 | 0 | 0 | −0.00029678 | 0 |
| 30927 | Snai3 | −0.00173286 | 0 | 0 | −0.00200524 | −0.00031525 | −0.00152793 | 0 |
| 56273 | Pex14 | −5.78E−05 | 0 | 0 | −0.00023571 | 0.000207267 | 0.000399636 | 3.60E−05 |
| 99382 | Abtb2 | 0.000915192 | 0 | 0 | 0.001720435 | 0.000632578 | 0.000785463 | 0.001911288 |
| 110751 | Adam33 | −0.00013627 | 0 | 0 | 5.93E−05 | −0.00012781 | −0.00125191 | −0.00082033 |
| 170744 | Tlr8 | 0.001261631 | 0 | 0 | 0.000738874 | 0.000672224 | 0.000788518 | 0.001176795 |
| 18516 | Pbx3 | −0.0011545 | 0 | 0 | 0.001609802 | 0.000833546 | −0.00079925 | 0.001118582 |
| 229658 | Vangl1 | −0.00115522 | 0 | 0 | −0.0015312 | −0.00141703 | −0.00069806 | −0.00153886 |
| 20209 | Saa2 | 0.001846707 | 0 | 0 | 0 | 0.001521289 | 0.001537762 | 0 |
| 213236 | Dnd1 | −0.00030242 | 0 | 0 | 0.000807816 | −0.00015797 | −0.00077567 | 0 |
| 67533 | Ppfibp1 | 0.000198882 | 0 | 0 | 0.001050817 | −1.52E−05 | 0.000626305 | −0.00046058 |
| 11484 | Aspa | −0.00075761 | 0 | 0 | 0.001089263 | 0.000617107 | −0.00120517 | 0.001055265 |
| 665033 | Col6a5 | −0.00056489 | 0 | 0 | −0.00013567 | −0.00054643 | −0.00092667 | 0.000308787 |
| 228421 | Kif18a | −0.00017418 | 0 | 0 | 0.000128657 | −0.00054671 | 0.000503742 | 2.75E−06 |
| 100038948 | Mup9 | 0 | 0 | 0 | 0 | 0 | −0.00161135 | 0 |
| 244416 | Ppp1r3b | −0.00043153 | 0 | 0 | 0.001004719 | 0.000741186 | −6.42E−05 | 0.000825619 |
| 107094 | Rrp12 | 5.66E−05 | 0 | 0 | −0.00076635 | −8.82E−05 | −0.00037048 | −0.00078082 |
| 17991 | Ndufa2 | 0.000708415 | 0 | 0 | −0.00029636 | −0.00016827 | −0.0001528 | −0.00075062 |
| 17167 | Marco | −0.00146141 | 0 | 0 | −0.00315113 | −0.00132659 | −0.00085807 | 0 |
| 22340 | Vegfb | 0.000270386 | 0 | 0 | 0.001741198 | 0.000958941 | −8.25E−05 | 0.000309802 |
| 15959 | Ifit3 | 0.000794421 | 0 | 0 | 0.000685461 | 0.0001001 | 5.88E−05 | 0.000632858 |
| 224481 | Tfb1m | −0.00021095 | 0 | 0 | 0.00056407 | 0.00023471 | 0.000324884 | 0.000375567 |
| 18750 | Prkca | −0.00175513 | 0 | 0 | −0.00266785 | −0.00075684 | −0.00082652 | −0.00118499 |
| 217316 | Slc16a5 | −0.00172749 | 0 | 0 | −0.00274604 | −0.00160135 | −0.00056599 | 0 |
| 54124 | Cks1b | −0.00040076 | 0 | 0 | −0.00177218 | −0.00027161 | −0.00033123 | −0.00026793 |
| 56321 | Aatf | 0.000763969 | 0 | 0 | 0.002021354 | 0.0012007 | 0.001239885 | 0.001740869 |
| 320204 | Etfbkmt | −0.00027149 | 0 | 0 | −0.00037125 | −0.00039415 | −0.00024585 | −0.00051033 |
| 69083 | Sult1c2 | 0.000485343 | 0 | 0 | 0 | 0.000546602 | 0.000374262 | 0 |
| 15903 | Id3 | 0.001360183 | 0 | 0 | 0.001797051 | 0.001244916 | 0.000612044 | 0.00168298 |
| 22042 | Tfrc | 4.15E−05 | 0 | 0 | 0.001772147 | −0.0011255 | 0.001631032 | 0.001513779 |
| 72056 | 1810055G02Rik | −0.00096657 | 0 | 0 | 0.000870667 | −0.00062046 | 0.00012708 | −0.0002411 |
| 55936 | Ctps2 | 0.000707021 | 0 | 0 | 0.000625817 | 0.000967243 | 0.000615291 | 0.000817795 |
| 80837 | Rhoj | −0.00135633 | 0 | 0 | −0.00063962 | −0.00066213 | −0.00073954 | 0.000434724 |
| 13712 | Elk1 | −0.00072123 | 0 | 0 | −0.00176918 | −0.00099727 | 0.000379015 | −0.00015901 |
| 13897 | Ces1e | 0.001006084 | 0 | 0 | 0.000511064 | 0.000385258 | 0.000808724 | 0 |
| 12489 | Cd33 | −8.32E−05 | 0 | 0 | −0.00134302 | −1.25E−06 | −1.71E−05 | −0.00026875 |
| 19265 | Ptprcap | 0.001215413 | 0 | 0 | −0.00053653 | 0.000168177 | 0.00111213 | 0 |
| 17254 | Slc3a2 | −0.0013756 | 0 | 0 | −2.47E−05 | 4.93E−05 | −0.00025867 | 6.27E−05 |
| 76792 | Spring1 | 0.000111459 | 0 | 0 | −0.00030761 | −0.00025814 | 0.001381208 | 0 |
| 19193 | Pipox | 0.0004428 | 0 | 0 | −0.00017598 | 0.000251457 | 0.000310442 | 1.21E−05 |
| 55947 | Ddre1a | 0.000240013 | 0 | 0 | −0.00154882 | −0.00029446 | 2.49E−05 | −0.00081291 |
| 71712 | Dram1 | 0.001133341 | 0 | 0 | −0.000242 | 8.21E−05 | 0.000720176 | 2.70E−05 |
| 11639 | Ak4 | 0.001089383 | 0 | 0 | 0.002689532 | 0.001003167 | 0.0008977 | 0.001108625 |
| 67776 | Vwa5a | 0.000362483 | 0 | 0 | 0.000196641 | 0.000482675 | 0.001394859 | 0.001439037 |
| 234463 | Tmem184c | −0.00037564 | 0 | 0 | −0.00111525 | −0.00114201 | 0.000314218 | 0.000517667 |
| 224019 | Tmem191c | 0.000281238 | 0 | 0 | 0.001134183 | 0.000473518 | −0.00067274 | 0 |
| 242705 | E2f2 | 0.000457156 | 0 | 0 | −0.00089776 | 0.000618972 | 0.00087916 | 0.000818161 |
| 100213 | Rusc2 | 0.000559879 | 0 | 0 | 0.000694615 | 0.000193239 | −0.00037629 | 0.000338955 |
| 245026 | Col6a6 | −0.00069902 | 0 | 0 | −0.00166607 | −0.00101405 | −0.00055793 | −0.00171385 |
| 226591 | Tiprl | 0.000121711 | 0 | 0 | −0.00054397 | −0.00049997 | 0.001001864 | 0.000772995 |
| 18441 | P2ry1 | −0.00035325 | 0 | 0 | −0.00211501 | −0.0005574 | −0.00064858 | −0.00135448 |
| 74430 | Cfap20dc | −0.00048771 | 0 | 0 | −0.00096245 | −0.00063967 | −0.0007276 | 0 |
| 71733 | Susd2 | 0.000166532 | 0 | 0 | −0.00024713 | 0.000947561 | 0.000314034 | 0.000105015 |
| 235431 | Coro2b | 0.000289525 | 0 | 0 | −0.00010895 | −0.00033539 | 8.83E−05 | −0.00040138 |
| 106947 | Slc39a3 | 8.06E−06 | 0 | 0 | −0.00115438 | −0.00082454 | 0.000106055 | −0.00090654 |
| 22687 | Zpr1 | 0.00039278 | 0 | 0 | 0.001903375 | 0.000786399 | 0.000296392 | 0.000965327 |
| 338467 | Morc3 | 0.001116342 | 0 | 0 | 0.000324822 | 0.000521057 | 0.001618006 | 0.00071759 |
| 449521 | Zfp213 | −5.21E−05 | 0 | 0 | 0.000261406 | 1.87E−05 | −0.00091208 | −9.33E−05 |
| 252838 | Tox | −0.00125309 | 0 | 0 | −0.00064576 | −0.00022132 | −0.00017451 | 0.001027522 |
| 67492 | Zfand4 | 0.001472762 | 0 | 0 | 0.003278946 | 0.001386391 | 0.001160959 | 0.001965114 |
| 74088 | Them7 | 0.000788874 | 0 | 0 | 0 | 0.000435685 | −3.83E−05 | 0 |
| 69123 | Eci3 | −0.00078031 | 0 | 0 | −0.00195094 | −0.00142149 | −0.00078088 | 0 |
| 432613 | Trim80 | 0.000527593 | 0 | 0 | 0 | −0.00035224 | 0.000430461 | 0 |
| 17295 | Met | 0.000657613 | 0 | 0 | −0.0017802 | −0.00034692 | −0.00013162 | −0.00129938 |
| 234671 | Ces2c | −0.00041111 | 0 | 0 | 0 | 0 | −0.00082181 | 0 |
| 170822 | Usp33 | −0.00037744 | 0 | 0 | −0.00102193 | −0.00070264 | 7.69E−05 | −0.0005753 |
| 73332 | Ccdc30 | −0.00114853 | 0 | 0 | −0.00084823 | −0.00044629 | 7.37E−05 | −0.00127013 |
| 207215 | Fbxo40 | 0.00160911 | 0 | 0 | 0.000243626 | −0.0001825 | 0.001115057 | −0.00137431 |
| 73205 | C9orf72 | −0.00158089 | 0 | 0 | −0.00131921 | −0.00066495 | −0.00016779 | 0.000500721 |
| 67609 | 4930453N24Rik | −0.00081965 | 0 | 0 | −0.00075248 | −0.00012643 | 0.000142361 | 0.000167336 |
| 259301 | Leap2 | 0.001009502 | 0 | 0 | 0 | 0.000956338 | 0.001374661 | 0 |
| 67460 | Decr1 | 0.001222073 | 0 | 0 | 0.000756558 | 0.001157356 | −0.00023237 | 0.000376279 |
| 28250 | Slco1e4 | −0.00010379 | 0 | 0 | −0.00139317 | 9.92E−05 | −0.00036581 | −0.00026587 |
| 80885 | Hcar2 | −0.00088975 | 0 | 0 | 0.000513758 | −0.00038789 | −0.00087862 | 0 |
| 97884 | B3galnt2 | −0.00045081 | 0 | 0 | −0.00097376 | −0.00028522 | −0.0003154 | −0.00116983 |
| 21346 | Tagln2 | 8.18E−06 | 0 | 0 | 0.000233319 | −1.19E−05 | 0.000622427 | 0.000704345 |
| 13682 | Eif4a2 | 3.11E−06 | 0 | 0 | −0.00202543 | −0.00069713 | 0.000153044 | −0.00095188 |
| 12765 | Cxcr3 | 0.000995216 | 0 | 0 | −0.00089264 | 0.00099027 | −0.0005223 | 0 |
| 77579 | Myh10 | −0.00169624 | 0 | 0 | −0.00128153 | −0.00082323 | −0.00052971 | −0.00038821 |
| 11676 | Aldoc | 0.001699257 | 0 | 0 | 0.002073445 | 0.001037171 | 0.000823055 | 0.001146912 |
| 71492 | Bbs7 | −0.00045277 | 0 | 0 | −3.28E−05 | −0.00030314 | −0.00042635 | −0.00050597 |
| 224170 | Dzip3 | −0.00088101 | 0 | 0 | −0.00337956 | −0.00128813 | −0.00113912 | −0.00215262 |
| 378937 | Lrrc24 | −1.48E−06 | 0 | 0 | −0.0006579 | −0.00024242 | 0.000427005 | 0.000152063 |
| 19707 | Reps1 | −0.00031604 | 0 | 0 | 0.000343785 | −3.36E−05 | 0.000657329 | 0.000608044 |
| 319530 | Zfp750 | −0.00012932 | 0 | 0 | 3.91E−05 | 0.000260737 | −0.00116119 | −0.00108266 |
| 14218 | Sh3pxd2a | −0.00018788 | 0 | 0 | −0.00011684 | 4.88E−05 | 0.000116436 | 0.000797768 |
| 66255 | Hsbp1l1 | −0.00026545 | 0 | 0 | −0.00108282 | −0.0003286 | 0.000656454 | 0 |
| 69430 | 1700048O20Rik | 0 | 0 | 0 | 0 | 0 | 1.00E−04 | 0 |
| 109323 | C1qtnf7 | −0.0002899 | 0 | 0 | −0.00037714 | −0.00036129 | −0.00033482 | −0.00058877 |
| 15331 | Hmgn2 | 0.000512592 | 0 | 0 | −0.00084907 | −0.00073513 | 0.000366087 | −0.00084351 |
| 74309 | Osbp2 | −0.00103034 | 0 | 0 | −0.00079972 | −0.00028325 | −0.00016139 | 0.000319864 |
| 19737 | Rgs5 | −0.00120084 | 0 | 0 | 0.000305031 | 0.000828337 | −0.00036353 | 0.001947442 |
| 12401 | Serpina6 | 0.000803311 | 0 | 0 | −0.00210079 | 1.21E−05 | 0.000573511 | 0 |
| 78779 | Spata2l | −0.00112076 | 0 | 0 | −0.00101873 | −0.00060595 | −0.00109275 | −0.00108093 |
| 17472 | Gbp4 | 0.000534932 | 0 | 0 | −0.00056492 | 0.000757528 | −0.00025457 | 0 |
| 381199 | Tmem151a | −0.00016317 | 0 | 0 | −0.00060563 | −0.00066343 | −0.00024801 | −0.0005434 |
| 24052 | Sgcd | 0.000118022 | 0 | 0 | −0.00066387 | −8.02E−06 | 0.001045201 | 0 |
| 231201 | Semp2l2a | 0.00066563 | 0 | 0 | 0 | 0 | 0.000470204 | 0 |
| 76633 | Lrmda | −0.00023259 | 0 | 0 | 0.000421284 | 0.000262405 | 0.000587138 | 0.000570499 |
| 12467 | Cct6b | −0.00059633 | 0 | 0 | 0.000957295 | 0.000240839 | −0.0001553 | 0.000250356 |
| 104871 | Spata7 | −0.0005555 | 0 | 0 | −0.00258774 | −0.00026499 | 0.000100819 | −0.00019172 |
| 319173 | H2ac10 | 0 | 0 | 0 | 0 | 0 | −0.00156976 | 0 |
| 353282 | Sfmbt2 | 0.000624181 | 0 | 0 | −0.00155344 | −0.00078325 | 0.000870513 | 0 |
| 216134 | Pdxk | −0.00058138 | 0 | 0 | 0.000278648 | 0.000463651 | −0.00101339 | 0.000137925 |
| 319942 | A530016L24Rik | −0.00034078 | 0 | 0 | 0.001134149 | −3.60E−05 | 0.000394899 | 0.000302899 |
| 15982 | Ifrd1 | 0.00019581 | 0 | 0 | −1.52E−05 | −3.31E−05 | 0.000985997 | 0.000835554 |
| 54139 | Irf6 | −0.00011082 | 0 | 0 | −0.00093225 | −0.00032223 | 0.001385006 | 0.00134588 |
| 13838 | Epha4 | −0.00118554 | 0 | 0 | −0.00152057 | −0.00039994 | −0.00087309 | −0.00157904 |
| 99929 | Tiparp | 0.00035026 | 0 | 0 | −0.00025611 | 0.000640703 | 0.001368306 | 0.001174932 |
| 73149 | Clec4a3 | −0.00056244 | 0 | 0 | 0.000198011 | 0.000529934 | −0.00053472 | 1.82E−05 |
| 16924 | Lnx1 | 0.001013898 | 0 | 0 | −0.0008104 | 0.000343435 | 0.000792542 | −0.00039377 |
| 100039054 | Mup12 | 0 | 0 | 0 | 0 | 0 | 0.000462294 | 0 |
| 207181 | Rbms3 | 0.000573826 | 0 | 0 | −0.00239779 | −0.00111143 | 0.000353902 | −0.00106652 |
| 70240 | Ufsp1 | −0.00053561 | 0 | 0 | 0.000958837 | 0.000984807 | −0.00124215 | 0.000194489 |
| 78558 | Htra3 | 0.001504918 | 0 | 0 | 0.001581739 | −0.00041772 | 0.00063306 | −0.00021032 |
| 22590 | Xpa | 0.001420119 | 0 | 0 | 0.000491382 | 0.000417464 | 0.001662857 | 0.00099365 |
| 75345 | Slamf7 | 0.000103404 | 0 | 0 | 0.000913133 | 0.00120981 | −0.00061143 | 0.000765736 |
| 218476 | Gcnt4 | −0.00055207 | 0 | 0 | 0 | 0 | 0.000523291 | 0 |
| 18763 | Odc1 | −0.00098684 | 0 | 0 | −0.00288848 | −0.00081758 | −0.00069821 | −0.00127352 |
| 192232 | Hps4 | −0.0010413 | 0 | 0 | −0.00124818 | −0.00118522 | −0.00040219 | −0.00087559 |
| 66309 | Tmem128 | −0.00042535 | 0 | 0 | −7.44E−05 | −0.0001392 | 0.000484345 | 0.000317556 |
| 14870 | Gstp1 | 8.24E−05 | 0 | 0 | 0.001153836 | 0.000426106 | −0.00128629 | 4.98E−05 |
| 18674 | Slc25a3 | −0.0009789 | 0 | 0 | −0.00043157 | −0.0001432 | −0.0002387 | −0.00011605 |
| 232341 | Wnk1 | 0.000786012 | 0 | 0 | 2.04E−05 | 0.000497782 | 0.000917466 | 0.000978867 |
| 18585 | Pde9a | −0.00028984 | 0 | 0 | −0.00112582 | −0.00046876 | 0.00075813 | −6.69E−05 |
| 14263 | Fmo5 | 0.000589234 | 0 | 0 | −0.0003955 | 0.001387429 | 0.000338298 | −0.00046398 |
| 14726 | Pdpn | 0.001114285 | 0 | 0 | 0.002168961 | 0.001403621 | 0.000516062 | 0.001217631 |
| 381549 | Zfp69 | −0.00039209 | 0 | 0 | −0.00032285 | −0.00031258 | −5.80E−05 | −0.00037253 |
| 27359 | Sytl4 | −0.00068538 | 0 | 0 | −0.00030288 | 2.41E−05 | −0.000153 | −0.00024996 |
| 216856 | Nlgn2 | 0.000381345 | 0 | 0 | 0.001485533 | 0.000867443 | −0.00032623 | 0.000550357 |
| 74023 | Rd3 | 0.001052818 | 0 | 0 | 0 | 0.000125015 | 0.001352266 | 0 |
| 16430 | Stt3a | −0.00055778 | 0 | 0 | −0.00050969 | −0.00035746 | 0.000170722 | −0.00032194 |
| 320747 | Lingo4 | −0.00107101 | 0 | 0 | 0 | −0.00021685 | −0.00049695 | 0 |
| 20516 | Slc20a2 | −0.00040379 | 0 | 0 | 0.001282016 | 2.79E−05 | 0.000489696 | 0.000326567 |
| 74153 | Uba7 | −0.00114871 | 0 | 0 | 0.000226375 | 0.000622689 | 0.000217681 | 0.001659472 |
| 79221 | Hdac9 | −2.02E−05 | 0 | 0 | 0.000213998 | −0.00021946 | −2.59E−05 | 4.92E−05 |
| 20522 | Slc23a1 | −0.00013468 | 0 | 0 | 0.000573037 | −0.00027841 | 0.000825604 | 0.000253776 |
| 19299 | Abcd3 | 0.000161833 | 0 | 0 | 0.000637045 | −1.90E−05 | −0.00139526 | −0.00064269 |
| 66939 | Aagab | 0.000389268 | 0 | 0 | 0.000628424 | 0.000110984 | 0.000756474 | 0.000697144 |
| 66153 | Fbxo36 | −0.00131192 | 0 | 0 | −0.0020712 | −0.00087209 | −0.00033791 | −0.00067149 |
| 11554 | Adrb1 | 0.000391806 | 0 | 0 | 0.001904457 | 0.000538163 | −0.00060946 | −0.00089262 |
| 223753 | Cerk | 0.001231285 | 0 | 0 | 0.000336393 | −5.77E−05 | 0.00139445 | 0.00037927 |
| 170768 | Pfkfb3 | 0.000446702 | 0 | 0 | −0.00116698 | −0.00020209 | −0.00030226 | −0.00102112 |
| 53978 | Lpar2 | 0.001408885 | 0 | 0 | 0.001639019 | 0.0007263 | 0.001345262 | 0 |
| 57784 | Bin3 | 1.14E−05 | 0 | 0 | 0.003148116 | 0.001266026 | 0.00110019 | 0.002009801 |
| 107022 | Gramd3 | −0.00039071 | 0 | 0 | −0.00126544 | −0.00091957 | −0.00066667 | −0.00173274 |
| 107373 | Fam111a | 2.77E−05 | 0 | 0 | −0.00128141 | −0.00037963 | 0.000634805 | −0.00040904 |
| 12409 | Cbr2 | −0.00026725 | 0 | 0 | 0.001027548 | −5.56E−05 | −0.0009789 | 0 |
| 19823 | Rnf7 | −0.00073621 | 0 | 0 | −0.00093104 | −0.00081448 | −0.00046643 | −0.00031434 |
| 77832 | Tchp | −0.00064264 | 0 | 0 | −0.00026116 | 0.000167625 | −0.00082966 | −0.0002047 |
| 228839 | Tgif2 | −5.66E−05 | 0 | 0 | 0.000360362 | −0.00052664 | −0.0001347 | 0 |
| 15109 | Hal | 0.000515547 | 0 | 0 | 0.00264014 | 0.000921275 | −0.00057436 | 0.002114894 |
| 56699 | Cdc42ep4 | −0.00107827 | 0 | 0 | −0.00201639 | −0.00035253 | −0.0014276 | −0.00101919 |
| 338362 | Ust | −9.01E−06 | 0 | 0 | −0.00067784 | −0.00060925 | −3.14E−05 | −0.00025623 |
| 67304 | 3110070M22Rik | −0.00045171 | 0 | 0 | 0 | 0 | −0.00015788 | 0 |
| 266645 | Acmsd | −0.0001842 | 0 | 0 | 0 | −0.00114467 | 0.000638197 | 0 |
| 68279 | Mcoln2 | −0.00038487 | 0 | 0 | −0.0008423 | 0.00074882 | 0.000188453 | 0 |
| 66377 | Ndufc1 | −0.00027713 | 0 | 0 | −0.00115833 | −0.00018331 | −0.00043343 | 0.000152849 |
| 103136 | Pwp1 | −0.00016542 | 0 | 0 | 0.000251626 | 0.000209887 | 0.000422373 | 0.00095379 |
| 242642 | Hpdl | −0.00045709 | 0 | 0 | −0.00132555 | −0.00034035 | −0.00160977 | 0 |
| 16373 | Irx3 | 0 | 0 | 0 | 0.002054025 | 0.000943436 | 6.75E−06 | 0.000371851 |
| 212398 | Frat2 | 0.000684952 | 0 | 0 | 0.000787578 | −0.00082757 | −0.00060498 | −0.00158436 |
| 74157 | Cmtr1 | 0.000338868 | 0 | 0 | −0.00086327 | 0.000429632 | 0.001123722 | 0.001183022 |
| 18578 | Pde4b | −0.00095116 | 0 | 0 | −0.00103221 | −0.00025844 | −6.31E−06 | 0.000290412 |
| 12223 | Btc | −0.00170281 | 0 | 0 | 0.000513174 | 0.000523826 | −0.00107451 | 0.001792953 |
| 57764 | Ntn4 | 0.000861656 | 0 | 0 | 0.003397085 | 0.001575283 | 0.000599184 | 0.002163431 |
| 14858 | Gsta2 | 0.000386602 | 0 | 0 | 0 | 0 | 0.000598971 | 0 |
| 245049 | Myrip | −0.00034475 | 0 | 0 | 0.001942428 | 0.001554821 | −0.00075348 | 0.001297776 |
| 382066 | Prdm10 | −0.00035807 | 0 | 0 | 0.002034468 | 0.000536974 | −0.00085984 | 9.43E−05 |
| 76686 | Clip3 | −0.00027034 | 0 | 0 | −0.00180833 | −0.00023985 | −0.00091308 | 0.00019506 |
| 226407 | Rab3gap1 | −0.00025805 | 0 | 0 | −0.00087241 | −0.0004088 | 0.000128324 | −0.00029319 |
| 67307 | Pbld2 | 0.000434009 | 0 | 0 | −0.0002516 | −0.00047138 | 0.000451205 | 0 |
| 100039139 | Ccdc152 | −0.00088083 | 0 | 0 | −0.00341162 | −0.00057189 | −0.00077936 | 0 |
| 20621 | Snn | 0.000579852 | 0 | 0 | 0.000203844 | −0.00034402 | 0.001355496 | 0.000426811 |
| 83382 | Siglece | −0.00023193 | 0 | 0 | 0.000205462 | −0.00010033 | −0.00046787 | 0.000229963 |
| 16428 | Itk | 0.001535565 | 0 | 0 | −0.00073307 | 2.04E−05 | 0.001219762 | 0 |
| 238803 | Zfp366 | −0.00060056 | 0 | 0 | −0.00098246 | −0.00027534 | −0.00137623 | −0.00076409 |
| 224598 | Zfp758 | 0.000349291 | 0 | 0 | −0.00097532 | −0.00026604 | −0.00022238 | −0.00050703 |
| 74413 | Tc2n | −0.00132521 | 0 | 0 | 0.000226714 | −0.00029378 | −0.00050816 | 0.000124891 |
| 70315 | Hdac8 | −0.00088393 | 0 | 0 | −0.00148596 | −0.00095551 | −0.00037204 | −0.00082444 |
| 68014 | Zwilch | 0.000224069 | 0 | 0 | 0.001680112 | 0.000309105 | −0.0008102 | −0.00032276 |
| 67487 | Dhx40 | −0.00034292 | 0 | 0 | −0.00104045 | −0.00097493 | 1.96E−05 | −0.00048931 |
| 22068 | Trpc6 | −0.00057957 | 0 | 0 | −0.00019171 | −0.0002634 | −0.00078444 | −0.00151783 |
| 20504 | Slc17a1 | −7.65E−05 | 0 | 0 | −0.00025627 | −3.58E−05 | 0.000582711 | −0.00029221 |
| 71145 | Scara5 | 0.000314549 | 0 | 0 | −0.0013242 | −0.00064248 | 0.000861848 | −0.00011667 |
| 74008 | Arsg | −0.00040123 | 0 | 0 | 0.000998673 | 0.000769483 | 0.000229061 | 0.000973559 |
| 102294 | Cyp4v3 | −0.00109761 | 0 | 0 | 6.67E−06 | 0.000215418 | −0.00072084 | 0.000847288 |
| 67171 | Dram2 | 0.001071881 | 0 | 0 | 0.00037143 | 0.000301188 | 0.000338239 | −0.00010604 |
| 20682 | Sox9 | 0.001775438 | 0 | 0 | 0.000242605 | 0.00117033 | 0.00037537 | 0.000874599 |
| | | | | | Lifespan- | Lifespan- | | |
| | | Lifespan- | | Lifespan- | adjusted age | adjusted age | | |
| | | adjusted age | Lifespan- | adjusted age | differential | differential | | |
| | | multi-tissue | adjusted age | differential | multi-tissue | multi-tissue | Lifespan | Lifespan |
| Entrez | Gene | mouse + rat | differential | multi-tissue | mouse + rat | mouse + rat + | liver | differential |
| ID | symbol | clock (log-log) | liver clock | mouse clock | clock | human clock | clock | liver dock |
| 12457 | Noct | 0.004900294 | 0.012910839 | 0.010791262 | 0.011797078 | 0.009238967 | 0 | 0 |
| 20860 | Sult1e1 | 0.003726648 | 0.014435926 | 0.010471203 | 0.01048258 | 0.007417542 | 0 | −0.08005851 |
| 331535 | Serpina7 | 0 | 0.010382777 | 0 | 0 | 0.007274378 | 0 | −0.08794704 |
| 17381 | Mmp12 | 0.004961955 | 0.008821232 | 0.005663778 | 0.007753419 | 0.007744825 | 0 | −0.087371 |
| 232174 | Cyp26b1 | 0.003192344 | 0.009286402 | 0.004121808 | 0.005047958 | 0.006534196 | 0 | −0.10427596 |
| 319476 | Lrtm1 | 0.002825946 | 0.009566339 | 0.004561948 | 0.006638056 | 0.005171034 | 0.084718634 | 0 |
| 16008 | Igfbp2 | 0.00191937 | 0.009676731 | 0.005547037 | 0.009804027 | 0.011543098 | 0.234940625 | 0.197712045 |
| 13082 | Cyp26a1 | 0.004634518 | 0.011665565 | 0.003843182 | 0.008664321 | 0.005169946 | 0 | −0.39728423 |
| 109901 | Cela1 | −0.00075587 | −0.00367805 | 0.006590215 | 0.003189995 | −0.00040299 | 0 | −0.28830819 |
| 16819 | Lcn2 | 0.004974266 | 0.009219484 | 0.003496186 | 0.001524233 | 0.003757576 | −0.05600528 | 0 |
| 12550 | Cdh1 | −0.00131438 | −0.00514715 | −0.00779785 | −0.00041145 | 0.000392636 | −0.08021389 | 0 |
| 66222 | Serpinb1a | 0.002950305 | 0.012817925 | 0.00446045 | 0.006950254 | 0.004784844 | 0.147368613 | 0.196954391 |
| 16006 | Igfbp1 | −0.00361291 | −0.00997618 | −0.00965298 | −0.00918045 | −0.00753435 | 0 | 0.589480255 |
| 11997 | Akr1b7 | 0.001908872 | 0.00829849 | −0.00355171 | 0.001498914 | 0 | 0 | 0 |
| 17748 | Mt1 | −0.00370542 | −0.00482003 | −0.00780687 | −0.00868121 | −0.00810918 | 0 | 0.070322783 |
| 57429 | Sult5al | −0.00114277 | −0.01196428 | −0.00493091 | −0.00330365 | −0.00220056 | 0 | 0 |
| 20201 | S100a8 | 0.006235511 | 0.010662709 | 0.015138671 | 0.01635223 | 0.009321381 | 0 | 0 |
| 68680 | Fitm1 | 0.001933017 | 0.010051369 | 0.002730942 | 0.006296502 | 0.007660125 | 0 | 0 |
| 93695 | Gpnmb | 0.011517638 | 0.008693947 | 0.01958134 | 0.021334543 | 0.018375958 | 0 | −0.0137061 |
| 94226 | S1pr5 | 0.003292747 | 0.007068256 | 0.006430167 | 0.006187913 | 0.004063851 | 0 | 0.131195269 |
| 16069 | Jchain | 0.010394561 | 0.006625275 | 0.007684167 | 0.008406597 | 0.009046547 | 0 | 0 |
| 27276 | Plekhb1 | 0.004926082 | 0.010373989 | 0.010173073 | 0.011028975 | 0.008779005 | 0 | 0 |
| 13119 | Cyp4a14 | 0.000353289 | 0.0050488 | 0.004169955 | 0.004243451 | 0 | 0 | 0.285929688 |
| 21987 | Tpd52l1 | 0.003632797 | 0.006550285 | 0.002458994 | 0.004909164 | 0.004595245 | 0 | 0 |
| 116939 | Pnpla3 | 0.001952866 | 0.009971183 | 0.006227156 | 0.005722223 | 0.003334272 | 0 | −0.14901483 |
| 16854 | Lgals3 | 0.005921062 | 0.005120341 | 0.006049671 | 0.008031906 | 0.008527614 | 0 | −0.05172579 |
| 71690 | Esm1 | 0.001508611 | 0.005687636 | 0.002040545 | 0.004423223 | 0.003158519 | 0 | 0 |
| 66438 | Hamp2 | −0.00097091 | −0.00012558 | −0.00142859 | −0.00080786 | 0 | 0 | −0.13103214 |
| 11833 | Aqp8 | 0.000286132 | −0.00742276 | −0.00202309 | −0.00052918 | 2.37E−05 | 0 | 0 |
| 51902 | Rnf24 | −0.00108845 | −0.00591874 | −0.00315495 | −0.00223203 | −0.00212948 | 0 | 0 |
| 21743 | Inmt | −0.00337342 | −0.00837808 | −0.00495504 | −0.0050188 | −0.00736519 | 0.125421583 | 0 |
| 17436 | Me1 | −0.00099271 | −0.00139294 | 0.000396255 | −0.00342951 | −0.00355036 | 0 | 0 |
| 19116 | Prlr | 0.003324621 | 0.009511497 | 0.000968418 | 0.002482215 | 0.004350435 | 0 | −0.12328786 |
| 14945 | Gzmk | 0.004409741 | 0.005829811 | 0 | 0.007549623 | 0.005794609 | 0 | 0 |
| 20775 | Sqle | 0.000880562 | 0.003069338 | 0.003169122 | 0.001339746 | 0.002971551 | 0 | 0.198346541 |
| 13370 | Dio1 | −0.00311049 | 0.000436449 | −0.00093316 | −0.00357818 | −0.0035218 | −0.03929809 | −0.53794749 |
| 19879 | Slc22a8 | −0.00267979 | −0.00639159 | −0.00138636 | −0.0057235 | −0.00441696 | 0 | 0 |
| 27409 | Abcg5 | −0.00238805 | −0.00245164 | 0.000872768 | −0.00231519 | −0.00301846 | 0 | 0 |
| 14825 | Cxcl1 | 0.002650212 | 0.002594453 | 0.000569446 | −3.90E−05 | 0.001425235 | 0 | 0.082054283 |
| 20304 | Ccl5 | 0.002938514 | 0.006317833 | 0.004900801 | 0.006901515 | 0.005272068 | 0 | 0 |
| 100043194 | Sult2a2 | 0 | −0.01301157 | 0 | 0 | 0 | 0.182227025 | 0.696407847 |
| 246277 | Csad | −0.00082985 | −0.00409021 | −0.00092694 | −0.00283985 | −0.00096855 | 0 | 0.001993519 |
| 72002 | Slc39a5 | −0.00241517 | −0.00421702 | −0.00429505 | −0.00587994 | −0.00502778 | 0 | 0 |
| 211187 | Lrtm2 | 2.41E−05 | 0.001976424 | −0.00380029 | −0.00160942 | −0.00049778 | 0 | 0.018248837 |
| 93671 | Cd163 | −0.00129693 | −0.00685853 | −0.00693809 | −0.00621072 | −0.00416072 | 0 | 0 |
| 13653 | Egr1 | 0.002523616 | 0.001399124 | −4.74E−05 | 0.001856168 | −0.0001148 | 0 | −0.21959513 |
| 28248 | Slco1a1 | 0 | −0.00450131 | 0 | 0 | 0 | 0 | 0.282474234 |
| 242509 | Bnc2 | 0.001887757 | 0.008551027 | 0.006413818 | 0.008436069 | 0.006300002 | 0 | 0 |
| 114886 | Cygb | 0.004034961 | 0.002114483 | 0.002821255 | 0.003288931 | 0.005008266 | 0 | 0 |
| 231691 | Sds | −0.00255535 | −0.00289303 | −0.00049364 | −0.00436192 | −0.00381425 | 0 | 0 |
| 70237 | Bhlhb9 | −0.0022478 | −0.00479061 | −0.00230958 | −0.00496385 | −0.00411796 | 0 | −0.01879934 |
| 69772 | Bdh2 | 0.001260082 | 0.002272772 | −0.0014529 | −0.00098397 | 0.001396221 | 0 | 0.266175159 |
| 14859 | Gsta3 | 0.00415688 | 0.008137176 | 0.002136469 | 0.007417359 | 0.006153138 | 0 | 0 |
| 13099 | Cyp2c40 | 0 | −0.00345286 | 0 | 0 | 0 | 0 | 0.119912965 |
| 216831 | Arhgap44 | 0.000537932 | −0.00510229 | −0.00511949 | −0.00435293 | −0.00124175 | 0 | 0 |
| 76560 | Prss8 | 0.002255393 | 0.002410897 | −0.00136545 | 0.000438329 | 0.002921736 | 0 | −0.01645367 |
| 12832 | Col5a2 | −0.00444333 | −0.00368993 | −0.0080615 | −0.00818382 | −0.00559694 | 0 | 0 |
| 20210 | Saa3 | 0.001451424 | 0.003981973 | 0.005399548 | 0.004915659 | 0 | 0 | 0.10810972 |
| 20200 | S100a6 | 0.003436158 | 0.003104168 | 0.008043704 | 0.008503945 | 0.007835097 | 0 | 0.117197831 |
| 654812 | Angptl7 | −0.00091734 | −0.00280173 | −0.00168296 | −0.00190366 | −0.00142686 | 0 | 0 |
| 319996 | Golm2 | −0.00211627 | −0.00561584 | −0.00429015 | −0.00582086 | −0.00339438 | 0 | 0 |
| 26875 | Pclo | 0.003367192 | 0.001066517 | 0.003532713 | 0.004051332 | 0.005029445 | 0 | 0 |
| 75772 | Pnpla5 | 0.001902216 | 0.004726402 | 0.002702804 | 0.004288873 | 0.002790918 | 0 | −0.23103801 |
| 16840 | Cnmd | 0.000895031 | 0.009213084 | 0.004497873 | 0.005077213 | 0.003171669 | 0 | 0 |
| 22127 | Tsx | 0 | 0.00503539 | 0 | 0 | 0 | 0 | 0.045903811 |
| 56744 | Pf4 | 0.000911364 | 0.00811315 | 0.003265208 | 0.001556188 | 0.002064739 | 0 | 0 |
| 211949 | Spsb4 | 0.001944353 | 0.006842016 | 0.006313455 | 0.006416546 | 0.005579513 | 0 | −0.04290524 |
| 140577 | Ankrd6 | 0.002362221 | 0.004933876 | 0.003740181 | 0.005962017 | 0.003760849 | 0 | 0 |
| 14803 | Grid1 | 0.002499999 | 0.002049032 | 0.004185058 | 0.003376904 | 0.003433005 | 0 | 0.102565492 |
| 230590 | Zyg11a | −0.00213257 | −0.00891442 | −0.00572124 | −0.00612027 | −0.00460764 | 0 | 0 |
| 105349 | Akr1c18 | −0.00238336 | −0.00625692 | −0.00342983 | −0.00610689 | −0.00405624 | 0 | 0 |
| 13117 | Cyp4a10 | 0 | 0.00310396 | 0 | 0 | 0 | 0 | 0 |
| 14137 | Fdft1 | 0.001091699 | 0.004750032 | 0.002251224 | 0.002908714 | 0.001315517 | 0 | 0 |
| 17387 | Mmp14 | 0.002564706 | 0.00167854 | 0.002416184 | 0.002664385 | 0.00445183 | 0 | 0 |
| 84112 | Sucnr1 | 0.001202088 | 0.008283165 | 0.004253016 | 0.001715897 | 0.001852459 | 0 | 0.09534543 |
| 68396 | Nat8 | 0.001131942 | 0.005331918 | 0.001534341 | 0.004446744 | 0 | 0 | 0.151947195 |
| 242523 | Dmrta1 | 0.002554759 | 0.002812947 | 0.002475371 | 0.002608909 | 0.006504336 | 0 | 0.324305859 |
| 59012 | Moxd1 | −0.0017239 | −0.00488523 | −0.00504798 | −0.00364282 | −0.00234804 | 0 | 0.283108845 |
| 20198 | S100a4 | 0.00327443 | 0.004612954 | 0.004542532 | 0.00892199 | 0.007371005 | 0 | 0 |
| 93694 | Clec2d | −0.00355513 | −5.42E−05 | −0.00293598 | −0.0024875 | 0 | 0 | 0 |
| 11611 | Agxt | 0.000346035 | 0.006640303 | 0.006262936 | 0.005593787 | 0.004613392 | 0 | −0.26260918 |
| 171405 | Slc22a27 | 0 | −0.0030604 | 0 | 0 | 0 | 0 | 0.055799815 |
| 56636 | Fgf21 | 0.001156861 | 0.008929065 | 0.006543218 | 0.00497912 | 0.00363725 | 0 | 0.081504241 |
| 57435 | Plin4 | −0.000948 | −0.00145196 | −0.00466127 | −0.00638892 | −0.00523034 | 0 | 0 |
| 246691 | Prok1 | 0 | 0.005542877 | 0.00695839 | 0 | 0.004394564 | 0 | 0 |
| 192156 | Mvd | 0.000922098 | 0.00527361 | 0.004688215 | 0.004686539 | 0.002226756 | 0 | 0 |
| 75487 | Ppp1r3g | 0.000800803 | −0.0045663 | −0.00153797 | −0.00267702 | 0.002226797 | 0 | 0 |
| 140904 | Caln1 | 0.001542327 | 0.004378526 | 0.000700307 | 0.003089828 | 0.002118303 | 0 | 0 |
| 52668 | Ifi27 | 0.003251686 | 0.003790059 | 0.00528469 | 0.007121345 | 0.00622926 | 0 | 0 |
| 15496 | Hsd3b5 | 0 | −0.00165655 | 0 | 0 | 0 | 0 | −0.10078918 |
| 16147 | Ihh | 0.001789278 | 0.003541085 | 0.006194565 | 0.008214035 | 0.006812369 | 0 | 0 |
| 21413 | Tcf4 | 0.001398266 | 0.004185916 | 0.003260711 | 0.003053325 | 0.000992562 | 0 | 0 |
| 27528 | Nrep | −0.00791284 | −0.00126359 | −0.00173848 | −0.00564008 | −0.00661988 | 0 | 0.014663415 |
| 114332 | Lyve1 | −0.0013728 | −0.00351294 | −0.00137818 | 0.000847047 | −0.00043303 | 0 | 0 |
| 624219 | Angptl8 | −0.00042216 | 0.004326863 | 0.00198606 | 0.003536655 | 0.00235088 | 0 | 0 |
| 69060 | Pnlip | −0.00102553 | −0.00614846 | −0.00219043 | −0.00265339 | −0.00152296 | 0 | 0.013383772 |
| 237611 | Stac3 | −0.00419643 | −0.00926489 | −0.00312756 | −0.00916773 | −0.00718829 | 0 | 0 |
| 17060 | Blnk | 0.002086483 | 0.007169563 | 0.002199706 | 0.003861908 | 0.004525009 | 0 | −0.09129983 |
| 67470 | Abcg8 | −0.00167042 | −0.00255787 | −0.00079081 | −0.00856626 | −0.00173677 | 0 | 0 |
| 12350 | Car3 | −0.00245775 | −0.00465908 | −0.0043028 | −0.00337837 | −0.0046419 | 0 | −0.23609592 |
| 18407 | Orm3 | 0.001809028 | 0.007897039 | 0.005969973 | 0.00508457 | 0 | 0 | 0 |
| 232400 | A2ml1 | 0.002998298 | 0.00040381 | −0.00096526 | 0.000618786 | 0.000985609 | 0 | 0.190567184 |
| 243755 | Slc13a4 | −0.002959 | −0.00299679 | 0.002001638 | −0.00114396 | −0.00235286 | 0 | 0 |
| 19152 | Prtn3 | 0.002980214 | −0.001498 | −0.00018084 | 0.000915371 | 0.001156557 | 0 | 0.02544578 |
| 76703 | Cpb1 | −0.00224522 | −0.00261299 | −0.00318709 | −0.00529512 | −0.0034861 | 0 | 0.15227343 |
| 234199 | Fgl1 | 0.002840594 | 0.003161113 | 0.001034952 | 0.001963687 | 0.001622701 | 0 | 0 |
| 15122 | Hba-a1 | −0.00382835 | 0.0012162 | −0.00070019 | −0.00321687 | −0.00170312 | 0 | 0 |
| 100034251 | Wfdc17 | 0.004157695 | 0.0012416 | 0.001411065 | 0.001017942 | 0.000988942 | 0 | 0 |
| 213649 | Arhgef19 | 0.002911821 | 0.003838337 | 0.004169647 | 0.005097128 | 0.003475936 | 0 | 0 |
| 17750 | Mt2 | −0.0022662 | −0.0074917 | −0.00309454 | −0.00297594 | 0 | 0 | 0 |
| 22242 | Umod | −0.00108816 | −0.00600802 | −0.00668443 | 5.55E−05 | −0.0014142 | 0 | −0.02053572 |
| 117586 | A1bg | 0 | −0.00084713 | 0 | 0 | −0.00275846 | 0 | 0.228651423 |
| 20463 | Cox7a2l | 0.001187813 | 0.00256551 | 0.001551038 | 0.001840095 | 0.001119759 | 0 | 0 |
| 66684 | Tceal8 | −0.00134775 | −0.00710754 | −0.00719843 | −0.00665215 | −0.00404011 | 0 | 0 |
| 72297 | B3gnt3 | 0.00074623 | 0.004973008 | 0.001227992 | 0.001233137 | 0.002115454 | 0 | 0 |
| 54613 | St3gal6 | 0.002337213 | 0.004350925 | 0.007225097 | 0.008599413 | 0.003986423 | 0 | 0 |
| 94179 | Krt23 | −0.00269995 | −0.00096912 | −0.00398851 | −0.00235799 | −0.00228812 | 0 | 0.164795441 |
| 55985 | Cxcl13 | 0.004810203 | −0.00671415 | 0.004736787 | 0.002551298 | 0.004513823 | 0 | 0.110590395 |
| 57742 | Abhd1 | −0.00268572 | −0.00968311 | −0.00802387 | −0.00863427 | −0.00537875 | 0 | 0 |
| 319317 | Snhg11 | 0.003548399 | 0.003746398 | 0.008004529 | 0.008551122 | 0.006924918 | 0 | 0 |
| 54123 | Irf7 | 0.002900835 | 0.003285816 | 0.005796735 | 0.004386693 | 0.004509812 | 0 | 0.057042681 |
| 15985 | Cd79b | −0.00105936 | −0.00372962 | −0.00548398 | −0.006856 | −0.00438295 | 0 | 0 |
| 66184 | Rps4l | −0.00250389 | −0.00736054 | −0.01084091 | −0.01189351 | 0 | 0 | −0.10861079 |
| 75178 | Meiob | 0 | −0.00366432 | −0.00307475 | 0 | −0.00165197 | 0 | 0 |
| 53614 | Reck | −0.00084615 | −0.00608834 | −0.00471909 | −0.00449991 | −0.00249298 | 0 | 0 |
| 13105 | Cyp2d9 | −0.00271195 | −0.00642475 | −0.00499681 | −0.00634938 | 0 | 0 | 0 |
| 11529 | Adh7 | 4.03E−05 | 0.002133964 | 9.35E−05 | 0.003155407 | 0.001694167 | 0 | 0.009985208 |
| 214523 | Tmprss4 | −0.00018595 | −0.00226173 | −0.00265486 | −0.0017683 | −0.00055235 | 0 | 0 |
| 235135 | Tmem45b | 0.001476375 | 0.003663177 | 0.006157407 | 0.008841949 | 0.005280189 | 0 | −0.04497754 |
| 21786 | Tff3 | 0.001103194 | −0.00095668 | 0.002644549 | 0.001249404 | 0.001141446 | 0.023409179 | 0.054875544 |
| 21673 | Dntt | 0.000669161 | 0.006610054 | 0.004514596 | 0.004491114 | 0.002925275 | 0 | −0.11877741 |
| 75104 | Mmd2 | 0.000721772 | 0.004218198 | 0.006523315 | 0.007509877 | 0.004247763 | 0 | 0 |
| 245527 | Eda2r | 0.003742262 | 0.002947367 | 0.005953695 | 0.005532673 | 0.007207803 | 0 | 0 |
| 14373 | G0s2 | 0.002563332 | −0.00337329 | 0.004267732 | 0.005594167 | 0.00350349 | 0 | 0 |
| 13116 | Cyp46a1 | 0.00102708 | 0.005628814 | 0.00637038 | 0.004144428 | 0.003187815 | 0 | 0 |
| 16841 | Lect2 | −0.00141301 | −0.00646987 | −0.00407174 | −0.00569101 | −0.00168772 | 0 | 0 |
| 20592 | Kdm5d | 0.00223292 | 0.001596595 | −0.00042446 | 0.000953571 | 0.000252414 | 0 | 0 |
| 22349 | Vil1 | 0.00329623 | 0.001383758 | 0.008729653 | 0.00524181 | 0.005267024 | 0 | 0 |
| 109660 | Ctrl | −0.00099716 | −0.00261302 | 0 | −0.00201845 | −0.00151883 | 0 | 0.000615112 |
| 193740 | Hspa1a | −0.00218637 | −0.00340136 | −0.0052153 | −0.00454691 | 0 | 0 | 0.089262887 |
| 12825 | Col3a1 | −0.00830283 | −0.00228625 | −0.00849419 | −0.01314114 | −0.01010792 | 0 | 0 |
| 12482 | Ms4a1 | 0.003356249 | 0.005048526 | 0.006513702 | 0.008764397 | 0.006365869 | 0 | 0 |
| 58176 | Rhbg | −0.00113725 | −0.00728066 | −0.00191658 | −0.00184949 | −0.00312603 | 0 | 0.0236457 |
| 67701 | Wfdc2 | 0.004032587 | 0.007704325 | 0.004783015 | 0.004824762 | 0.004043735 | 0 | −0.26214925 |
| 26877 | B3galt1 | 0.001757159 | 0.004177278 | 0.00096761 | −0.001607 | −0.00126945 | 0 | 0 |
| 15139 | Hc | 0.000627412 | 0.003342583 | −6.58E−05 | 0.002527666 | 0.000813405 | 0 | 0.015967229 |
| 94185 | Tnfrsf21 | 0.000373669 | 0.004104592 | −0.00014141 | −0.00046464 | −0.00045151 | 0 | 0 |
| 26900 | Ddx3y | 0.002547373 | 0.001990109 | 0.002728166 | 0.001674502 | −0.00131153 | 0 | 0.003906745 |
| 12613 | Cel | −0.00074043 | −0.00323764 | −0.00147052 | −0.00426193 | −0.00044245 | 0 | 0 |
| 16847 | Lepr | 0.000572788 | −0.00317019 | 0.001345769 | 0.000135646 | 0.001707314 | −0.41789795 | −0.19434279 |
| 18414 | Osmr | 0.003966883 | 0.003296721 | 0.005807747 | 0.007661589 | 0.005658334 | 0 | 0 |
| 17841 | Mup2 | 0 | −0.00434306 | 0 | 0 | 0 | 0 | 0.150702141 |
| 12934 | Dpysl2 | 0.001409209 | 0.003784381 | 0.00051624 | 0.001553579 | 0.000196532 | 0 | 0 |
| 12332 | Capg | 0.002470094 | 0.006819432 | 0.008113055 | 0.009935676 | 0.005605021 | 0 | 0 |
| 105348 | Golm1 | −0.00040404 | −0.0019357 | −0.00184847 | −0.00136511 | −0.00028374 | 0 | 0 |
| 53973 | Cyp3a41a | 0 | −0.00124559 | 0 | 0 | 0 | 0 | 0 |
| 72310 | Nkg7 | 0.002340817 | −0.00100207 | −0.00216018 | −0.00067521 | 0.000453995 | 0 | 0 |
| 71461 | Ptk7 | −0.00246355 | −0.00297608 | −0.004308 | −0.00433284 | −0.00507102 | 0 | 0 |
| 228966 | Ppp1r3d | 0.00052082 | 0 | −0.00032327 | 0.000563579 | −0.00119711 | 0 | 0 |
| 94353 | Hmgn3 | 0.000394706 | 0.001292859 | 0.000384225 | 0.001958105 | 0.001919633 | 0 | 0 |
| 109821 | F11 | −0.0005407 | −0.00127384 | −0.00214377 | −0.0026135 | −0.00205278 | 0 | 0 |
| 226041 | Pgm5 | 0.001551882 | 0.004473825 | 0.003908646 | 0.003184877 | 0.002507567 | 0 | 0.007902693 |
| 11717 | Ampd3 | 0.00279765 | 0.002824184 | 0.002121021 | 0.003463112 | 0.002858789 | 0 | 0.09609399 |
| 26876 | Adh4 | 0 | 0.004931692 | 0.002620975 | 0 | 0.000511614 | 0 | 0.032662358 |
| 213742 | Xist | −2.58E−05 | −0.00016028 | −0.00129622 | −0.00238352 | −0.00112153 | −0.00231564 | 0 |
| 17189 | Mb | −0.00130028 | −0.00544492 | −0.00324379 | −0.00630438 | −0.00461925 | 0 | 0 |
| 11472 | Actn2 | −0.00128237 | −0.0026762 | −0.00299677 | −0.00094284 | −0.0010547 | 0 | 0 |
| 76982 | Vxn | 0.001455459 | 0.003114555 | −0.00058765 | 0.002293053 | 0.000960235 | 0 | 0 |
| 74182 | Gpcpd1 | −0.00202864 | −0.00361637 | −0.00425544 | −0.00511243 | −0.00530648 | 0 | 0 |
| 230822 | Ncmap | 0 | 0.002556171 | 0.003680853 | 0 | 0 | 0 | 0.281322905 |
| 50914 | Olig1 | 0.001799348 | 0.007377765 | 0.00529859 | 0.005848657 | 0.001413594 | 0 | 0 |
| 73690 | Glipr1 | 0.002178719 | 0.003060127 | 0.002800322 | 0.004041348 | 0.002843641 | 0 | 0 |
| 16643 | Klrd1 | 0.002150744 | 0.005118348 | 0.001325554 | 0.007154929 | 0.002953942 | 0 | 0 |
| 14977 | Slc39a7 | 0.000363034 | 0.003607405 | 0.001844533 | 0.002636757 | 0.000712115 | 0 | 0 |
| 56727 | Miox | −0.00158674 | −0.00430236 | −0.00536521 | −0.00585138 | −0.00229833 | 0 | 0 |
| 620807 | Mup6 | 0 | −0.00325248 | 0 | 0 | 0 | 0 | 0 |
| 59056 | Evc | 0.000896914 | −0.00122339 | −0.00267992 | −3.83E−05 | 0.000622783 | 0 | 0 |
| 12525 | Cd8a | 0.001129943 | 0.00436467 | 0.003610089 | 0.008083885 | 0.003520078 | 0 | 0 |
| 15894 | Icam1 | 0.002767975 | 0.003398891 | 0.003343067 | 0.005755025 | 0.006123777 | 0 | 0 |
| 66180 | P3h4 | −0.00072995 | −0.00196569 | −0.00065283 | −0.00329114 | −0.00246292 | 0 | 0 |
| 57738 | Slc15a2 | 0.000797682 | −0.0042997 | −0.00217199 | −0.00150772 | −0.00031201 | 0 | 0.156958116 |
| 83433 | Trem2 | 0.004080812 | 0.001520936 | 0.002019026 | 0.001276199 | 0.002813664 | 0 | 0.005814858 |
| 13713 | Elk3 | 0.001177812 | 0.003293531 | 0.001838371 | 0.002351654 | 0.002227015 | 0 | 0 |
| 231510 | Gpat3 | −0.00132511 | −0.00149178 | −0.00115849 | −0.00189578 | −0.00166677 | 0 | −0.01888525 |
| 15490 | Hsd17b7 | 0.001805099 | 0.004418546 | 0.005006944 | 0.004734307 | 0.002602405 | 0 | 0 |
| 60599 | Trp53inp1 | −0.00037649 | 0.000951731 | 0.001578735 | −1.79E−05 | 0.001205298 | 0 | −0.20146003 |
| 17897 | Myl3 | −0.00230633 | −0.00270353 | −0.0048954 | −0.00367964 | −0.00228481 | 0 | 0 |
| 239037 | Lrit1 | −0.00090045 | −0.00763028 | −0.00592061 | −0.00514179 | −0.00310687 | 0 | 0 |
| 105727 | Slc38a1 | −0.00490658 | −0.0051285 | −0.01155763 | −0.01245768 | −0.00960578 | 0 | 0 |
| 55193 | Plek | 0.003388495 | 0.005882683 | 0.007513951 | 0.002695134 | 0.003267963 | 0 | 0 |
| 56485 | Slc2a5 | 0.000559934 | 0.005440761 | −0.00212172 | 0.002998981 | 0.002532986 | 0 | 0.14140358 |
| 13197 | Gadd45a | 0.001944858 | 0.001136687 | 0.002361549 | 0.0032281 | 0.000775751 | 0 | −0.12570924 |
| 241447 | Cers6 | −0.0018256 | −0.00318377 | −0.00291148 | −0.00634952 | −0.0040386 | 0 | −0.05546024 |
| 16323 | Inhba | 0.001731078 | 0.001788623 | −0.00126089 | −0.00096465 | 0.001563566 | 0 | 0 |
| 14056 | Ezh2 | −0.00305637 | −0.00481728 | −0.00758375 | −0.00805952 | −0.00386027 | 0 | 0 |
| 13041 | Ctsw | 0.000811818 | −0.00040118 | 0.00026888 | 0.004570789 | 0.002246754 | 0 | 0.106532161 |
| 16427 | Itih4 | −0.00104166 | 0.001854758 | −0.00344159 | −0.0015753 | −0.00056319 | 0 | 0 |
| 13121 | Cyp51 | −0.00040341 | 0.002094979 | 9.11E−05 | −0.00069154 | 0.000119738 | 0 | 0.022063727 |
| 328977 | Zfp532 | −0.0021246 | −0.00418339 | −0.00231147 | 0.00308863 | −0.00326784 | 0 | 0 |
| 319554 | Idi1 | −0.00010902 | 0.003400155 | 0.000555611 | 0.004099912 | 0.001481425 | 0 | 0 |
| 276891 | Timd4 | −0.00311353 | −0.00569814 | −0.00686831 | −0.00581816 | −0.00454186 | 0 | 0 |
| 19734 | Rgs16 | 0.000569605 | 2.04E−05 | −0.00434152 | −0.00310122 | −0.00209987 | 0 | 0 |
| 11464 | Actc1 | −0.00178832 | −0.00237245 | −0.00368546 | −0.0033424 | −0.0008231 | 0 | 0 |
| 112407 | Egln3 | 0.003026036 | 0.003471115 | 0.007034436 | 0.007816766 | 0.005991961 | 0 | 0 |
| 230899 | Nppa | −0.00093884 | −0.00307745 | −0.01055907 | −0.01141609 | −0.00880724 | 0 | 0 |
| 171285 | Havcr2 | 0.00117453 | 0.002386216 | 0.00246199 | 0.002927766 | 0.001524122 | 0 | 0 |
| 20392 | Sgce | −0.00081083 | −0.00388915 | −0.0021044 | −0.00332586 | −0.00353274 | 0 | −0.04014486 |
| 25464 | Vnn3 | −0.00126507 | −0.00381701 | −0.00210808 | −0.00324991 | −0.00213727 | 0 | 0 |
| 19215 | Ptgds | 0.000403583 | −0.00425909 | −0.00304465 | −0.00243022 | 0.000305733 | 0 | 0 |
| 14027 | Evpl | 0.001184677 | −4.29E−05 | 0.001006764 | 0.000951315 | 0.001091147 | 0 | 0.079369131 |
| 12053 | Bcl5 | −0.00159248 | −0.00449518 | −0.00240253 | −0.00344229 | −0.00203313 | 0 | 0.157622949 |
| 230558 | C8a | −0.00123838 | −0.00232198 | −0.00069114 | −0.00233606 | −0.00089203 | 0 | 0 |
| 13649 | Egfr | −0.00219518 | −0.00259328 | −0.0043947 | −0.00441554 | −0.00265889 | −0.4445319 | 0 |
| 74020 | Cpne4 | 0.003517761 | 0.001759084 | 0.003915037 | 0.00812392 | 0.006830891 | 0 | 0 |
| 241633 | Atp8b4 | 0.001527128 | 0.003264482 | 0.006090754 | 0.004803958 | 0.003523087 | 0 | −0.03248508 |
| 22758 | Zscan12 | 0.002152357 | 0.003473939 | 0.005056421 | 0.004960059 | 0.003655427 | 0 | 0 |
| 26357 | Abcg2 | −0.00137802 | −0.00502558 | −0.00449104 | −0.00439948 | −0.00300482 | 0 | 0.069650702 |
| 227231 | Cps1 | −0.00052797 | 0.002642911 | 0.00521931 | 0.003437415 | 0.004804609 | 0 | 0 |
| 231633 | Tmem119 | −0.00175202 | −0.00450579 | 0.001495829 | 0.003458263 | 0.001168645 | 0 | 0 |
| 73673 | Rec114 | 0.000176844 | 0.00044934 | 0.00332446 | −0.00225859 | −0.00048937 | 0 | 0 |
| 12512 | Cd63 | 0.002541118 | 0.002998886 | 0.00632293 | 0.008744923 | 0.007055374 | 0 | 0 |
| 70676 | Gulp1 | −0.00022644 | 0.00465618 | −0.00014621 | −0.00062743 | −0.00012187 | 0 | 0 |
| 21408 | Zfp354a | −0.00180301 | −0.00462063 | −0.00251591 | −0.005217 | −0.00439417 | 0 | 0.011290942 |
| 20537 | Slc5a1 | 0.001698944 | 0.005637949 | 0.006455071 | 0.002659957 | 0.001832488 | 0 | −0.0910731 |
| 14685 | Gnat1 | 0.00078616 | 0.007280471 | 0.003516233 | 0.002465487 | 0.003835222 | 0 | 0 |
| 66433 | Chchd7 | −0.00023141 | −0.00625442 | 0.00068668 | −0.00063056 | −0.00021012 | 0 | 0 |
| 26362 | Axl | 0.001635673 | 0.002328858 | 0.00407392 | 0.003285961 | 7.88E−05 | 0 | 0 |
| 14734 | Gpc3 | 0.001511756 | 0.005597807 | 0.008939371 | 0.009608763 | 0.007865884 | 0 | −0.00430854 |
| 109225 | Ms4a7 | 0.004703829 | 0.004045466 | 0.010073659 | 0.007370849 | 0.006732833 | 0 | 0 |
| 104910 | Slc25a47 | 0.000744008 | 0.008055962 | 0.005461966 | 0.006283845 | 0.003328773 | 0 | 0 |
| 214854 | Neurl3 | 0.000276488 | 0.001847386 | −0.00504866 | −0.00221119 | 0.000494867 | 0 | 0 |
| 26358 | Aldh1a7 | 0.000835333 | 0.003231073 | 0.001945706 | 0.00094736 | 0.003326902 | 0 | 0.054225304 |
| 625098 | Slc38a6 | −0.00269057 | −0.00390762 | −0.00277869 | −0.002178 | −0.00126303 | 0 | 0 |
| 56644 | Clec7a | 0.006020088 | 0.003389687 | 0.014770271 | 0.014014698 | 0.0097483 | 0 | 0 |
| 13112 | Cyp3a11 | −0.00144167 | −0.00230296 | −0.00419208 | −0.00349857 | 0 | 0 | 0 |
| 67305 | Gpx7 | −0.00300385 | −0.00027622 | −0.00244697 | −0.00291971 | −0.00348582 | 0 | 0 |
| 20343 | Sell | −0.00270425 | −0.00250263 | −0.00499816 | −0.00965353 | −0.00514139 | 0 | −0.07640279 |
| 14115 | Fbln2 | −0.00137754 | −0.00086812 | −0.00317906 | −0.00660136 | −0.00613842 | 0 | 0 |
| 110893 | Slc8a3 | 0.003976897 | 0.003996206 | 0.005898115 | 0.007295065 | 0.003379047 | 0 | 0 |
| 14677 | Gnai1 | 1.08E−05 | 0.001026947 | 0.000653363 | 0.00096288 | 0.000926796 | 0 | 0 |
| 80859 | Nfkbiz | 0.001769505 | 0.004795851 | 0.001788381 | 0.003926761 | 0.002753766 | 0 | 0 |
| 20519 | Slc22a3 | −0.00184193 | −0.0040061 | −0.0045524 | −0.00332343 | −0.003799 | 0 | 0 |
| 15792 | Laptm5 | 0.002963741 | 0.004487251 | 0.002646196 | 0.004271746 | 0.003154124 | 0 | 0 |
| 211151 | Churc1 | 0.001659027 | 0.001362942 | −0.00111954 | −0.00166923 | 0.003342885 | 0 | 0 |
| 18294 | Ogg1 | −0.00101185 | −0.00376219 | −0.00287221 | −0.00491125 | −0.0025228 | 0 | 0 |
| 70080 | Igsf23 | 0 | 0.003190433 | −0.00071212 | 0 | −0.00095172 | 0 | −0.03085362 |
| 219151 | Scara3 | −0.00046257 | −0.00050882 | −0.00157828 | −0.00195046 | −0.00273862 | 0 | 0 |
| 18782 | Pla2g2d | 0.003202017 | 0.004480587 | −0.00278843 | 0.000727162 | 0.000168356 | 0 | 0 |
| 12274 | C6 | 0.001778372 | 0.010068752 | −0.00210952 | −0.00262715 | 0.001397483 | 0 | 0 |
| 217845 | Ifi27l2b | 0.000284859 | 0.002470839 | 0.000306929 | 0.00012093 | 0 | 0 | 0.018972189 |
| 666168 | Cyp4a31 | 0 | −0.00254526 | 0 | 0 | 0 | −0.12418098 | 0 |
| 20370 | Sez6 | −0.0007797 | 0 | −0.0013846 | −0.00169867 | −0.00146094 | 0 | 0 |
| 15201 | Hells | 0.000348448 | 0.008005164 | 0.004366939 | 0.005154472 | 0.003274433 | 0 | 0 |
| 17869 | Myc | 0.002712381 | 0.002377503 | 0.003693062 | 0.003143456 | 0.004242872 | −0.3023395 | −0.12688919 |
| 12683 | Cidea | 0.003724262 | 0.001116535 | 0.001092559 | 0.002365537 | 0.002504533 | 0 | −0.23665363 |
| 18703 | Pigr | 0.002608551 | 0.002181585 | 0.003424839 | −0.00103441 | 0.000572598 | 0 | 0 |
| 56264 | Cpxm1 | −0.0013796 | −0.00157228 | −0.00380646 | −0.00377046 | −0.00235545 | 0 | 0 |
| 278180 | Vsig4 | 0.005880228 | 0.00056933 | −0.00187672 | 0.000124333 | 0.001156017 | 0 | 0 |
| 15493 | Hsd3b2 | −0.0010289 | −0.00447334 | −0.00236812 | −0.00412235 | 0 | 0 | 0 |
| 171580 | Mical1 | −0.00063684 | −0.00608031 | −0.00397061 | −0.00556932 | −0.00191961 | 0 | 0 |
| 224440 | Setd4 | 0.000881899 | 0.005962798 | 0.003192243 | 0.002992748 | 0.001709523 | 0 | 0 |
| 268354 | Tafa2 | 0 | 0 | 0 | 0 | −0.00047835 | 0 | 0 |
| 12227 | Btg2 | 0.002018177 | 0.003408318 | 0.007079255 | 0.008754801 | 0.00525707 | 0 | 0 |
| 12364 | Casp12 | 0.002312129 | 0.00032079 | 0.006459604 | 0.009993397 | 0.007718129 | 0 | 0 |
| 12260 | C1qb | 0.003513155 | 0.001378192 | 0.003970289 | 0.002718944 | 0.000410515 | 0 | 0 |
| 25388 | Ifi202b | 0 | −0.00203935 | −0.00275545 | 0 | 0 | 0 | 0.006307707 |
| 18573 | Pde1a | 0.000983676 | 0.000545645 | 0.001530751 | 0.000455628 | 0.003284642 | 0 | 0 |
| 66098 | Chchd6 | −0.00072317 | −0.00333499 | −0.00301754 | −0.00304708 | −0.00315289 | 0 | 0 |
| 17868 | Mybpc3 | −0.00168809 | −0.00163875 | −0.00031331 | 0.002073052 | 0.000483772 | 0 | 0 |
| 70235 | Poc1a | −0.00110024 | −0.00262739 | −0.00108965 | 0.000372683 | −0.0013209 | 0 | 0 |
| 230971 | Megf6 | −0.0013377 | −0.00102114 | −0.00277218 | −0.00324262 | −0.00384083 | 0 | 0 |
| 233806 | Tmem159 | −0.00137211 | −0.0030926 | −0.00149962 | −0.0024219 | −0.00049373 | 0 | 0 |
| 76850 | Ago4 | 0.000587942 | −0.00266326 | 0.003677179 | 0.003163618 | 0.001049417 | 0 | 0 |
| 19348 | Kif20a | −0.00082141 | 0.002108563 | 0.002731937 | 0.001052295 | 0.001461439 | 0 | 0 |
| 667977 | Gm8909 | 0 | −0.00504014 | 0 | 0 | 0 | 0 | 0.026441191 |
| 110749 | Chaf1b | 0.000184443 | −0.00245086 | −0.00304373 | −0.00050591 | −0.00213106 | 0 | −0.02008046 |
| 20296 | Ccl2 | 0.001788542 | 0.000735628 | 0.000458008 | −0.00031501 | −0.00173884 | 0 | 0.25864487 |
| 70546 | Zdhhc2 | −0.00015897 | −0.00355859 | −0.00470829 | −0.00388092 | −0.00300607 | 0 | 0 |
| 269633 | Wdr85 | 4.64E−05 | 0.000834685 | 0.000731414 | 0.000422231 | −0.00157109 | 0 | 0.174685358 |
| 16541 | Napsa | −0.00106659 | −0.00217445 | −0.00728255 | −0.00654646 | −0.00466414 | 0 | 0 |
| 56185 | Hao2 | −0.00297494 | 0.004909781 | 0.00031491 | 0.000694481 | −0.00144746 | 0 | 0.305400634 |
| 230806 | Crybg2 | 0.002195749 | 0.002190419 | 0.00331309 | 0.003111836 | 0.003268123 | 0 | 0 |
| 24001 | Tlam2 | 0.002390496 | 0.004003807 | 0.006500804 | 0.003031839 | 0.002001444 | 0 | 0.079410336 |
| 26557 | Homer2 | 0.000205175 | −0.00518922 | 0.000841117 | −0.00026373 | 0.0002806 | 0 | 0 |
| 20208 | Saa1 | 0 | −0.00195481 | 0.001575299 | 0 | 0 | 0 | −0.00140028 |
| 268780 | Egflam | −0.00038612 | 0.003972583 | 2.45E−05 | 0.000162887 | 0.001574224 | 0 | 0 |
| 381801 | Tatdn2 | −0.00123037 | 0.000817505 | −0.00048998 | −0.0024171 | −0.00161634 | 0 | 0 |
| 231655 | Oasl1 | 0.002119892 | 0.005147852 | −0.00552759 | −0.00465334 | −0.00062374 | 0 | −0.06480005 |
| 56077 | Dgke | −0.00051675 | −0.00505061 | −0.00277991 | −0.00265877 | −0.00288317 | 0 | 0 |
| 67379 | Dedd2 | −0.00096413 | −0.00355402 | −0.00439913 | −0.00473582 | −0.00190581 | 0 | 0 |
| 68631 | Cryl1 | 0.000596303 | 0.001993312 | −0.00400384 | −0.00058702 | 0.000888933 | 0 | 0 |
| 217593 | Slc25a21 | −0.00110141 | −0.00447539 | −0.00150345 | −0.00199567 | 0.000196042 | 0 | 0 |
| 16716 | Ky | −0.00029175 | 0.004508799 | −0.00308888 | −0.00361123 | −0.00144142 | 0 | 0 |
| 14060 | F13b | 0.00010881 | 0.00172892 | −0.00148858 | −0.00237669 | −0.00153338 | 0 | 0 |
| 13123 | Cyp7b1 | −0.00018922 | 0.001337535 | −0.00520763 | −0.00489688 | −0.00365728 | 0 | 0.040275814 |
| 215690 | Nav1 | 0.000754905 | 0.003058287 | 0.002393307 | 0.003450829 | 0.001717388 | 0 | 0 |
| 14733 | Gpc1 | 0.002446972 | 0.006983631 | 0.006010581 | 0.005144196 | 0.004805215 | 0 | −0.21651485 |
| 19221 | Ptgfrn | −0.00115761 | −0.00330464 | −0.00338352 | −0.00228424 | −0.00220428 | 0 | 0 |
| 67017 | Fam210b | −0.00020895 | −0.00079038 | 0.003244986 | 0.002816656 | 0.001281425 | 0 | 0 |
| 19387 | Rangap1 | −0.001536 | −0.00099731 | −0.00199508 | −0.00094257 | −0.00090237 | 0 | 0 |
| 66395 | Ahnak | 0.002166742 | 0.003571647 | 0.004509061 | 0.002387908 | 0.006454545 | 0 | 0 |
| 114601 | Ehbp1l1 | 0.00190313 | 0.001905752 | 0.000457003 | 0.000620445 | 0.002106486 | 0 | 0 |
| 18187 | Nrp2 | 0.0017777 | 0.000476824 | 0.002083729 | 0.000726435 | 0.001325798 | 0 | 0 |
| 50781 | Dkk3 | 0.002583466 | 0.002201782 | 0.004886383 | 0.004671228 | 0.004474144 | 0 | 0 |
| 12523 | Cd84 | 0.002712957 | 0.00306629 | 0.00482615 | 0.006332341 | 0.003524547 | 0 | 0 |
| 53322 | Nucb2 | −0.00156717 | −0.00268349 | −0.00122937 | −0.00062235 | −0.00143801 | −0.00055124 | 0 |
| 71373 | Prr16 | 2.41E−05 | −0.00928255 | −0.00115132 | −0.00070341 | −6.09E−05 | 0 | 0 |
| 225884 | Gstp3 | 0 | −0.00207521 | 0 | 0 | 0 | 0 | 0 |
| 17855 | Mvk | 0.000499275 | 0.00505156 | 0.002716681 | 0.001974565 | 0.001685521 | 0 | 0 |
| 17105 | Lyz2 | 0.002022886 | 0.00525136 | 0.00291106 | 0.001094061 | 0.002562547 | 0 | 0 |
| 14823 | Grm8 | 0.000688523 | 0.000721049 | −0.00093431 | −0.0013409 | −0.00012304 | 0 | 0.060771385 |
| 20500 | Slc13a2 | −0.00120848 | −0.00578486 | −0.0040913 | −0.00569822 | −0.00422158 | 0 | 0 |
| 19736 | Rgs4 | −0.00072153 | 0.006625831 | 0.000218978 | −0.00216423 | −0.00267153 | 0 | 0 |
| 26897 | Acot1 | −0.0021244 | −0.00176042 | −0.00173398 | −0.00248183 | −0.00265462 | 0 | −0.07115004 |
| 20532 | Slc3a1 | 0.002031856 | 0.003526023 | 0.004038222 | 0.004105928 | 0.004324321 | 0 | −0.29182702 |
| 17318 | Mid1 | −0.00021614 | −0.00058826 | −0.00102694 | −0.00083938 | −0.00086729 | 0 | 0 |
| 93674 | Nat8f3 | 0 | −0.00543484 | 0 | 0 | 0 | 0 | 0.041943257 |
| 16371 | Irx1 | 0.001096864 | 0.000540659 | 0.004913961 | 0.003034344 | 0.004326879 | 0 | 0 |
| 13476 | Reep5 | −0.0009648 | −0.00128491 | −0.00238968 | −0.00298086 | −0.00359614 | 0 | 0 |
| 11826 | Aqp1 | 0.000553702 | 0.004381478 | 0.000326994 | 6.19E−05 | −0.00298554 | 0 | −0.02601411 |
| 71770 | Ap2b1 | −0.00184054 | −0.00280101 | −0.00056788 | −0.00028739 | −0.00167403 | 0 | 0 |
| 65256 | Asb2 | −0.00090477 | −0.0021315 | −0.00506699 | −0.00383688 | −0.00322829 | 0 | 0 |
| 214301 | Crygn | −0.0011904 | −0.00257645 | −0.0056306 | −0.00519309 | −0.00021915 | 0 | 0 |
| 69638 | Enho | 0.000828893 | 0.0012771 | 0.004262433 | 0.004418007 | 0.002999314 | 0 | 0 |
| 56642 | Ankrd2 | −0.00070948 | −0.00032463 | −0.0002828 | −0.00329914 | −0.00243375 | 0 | 0.016174455 |
| 231130 | Tnip2 | −0.00039172 | −0.00249755 | −0.00386538 | −0.0047754 | −0.00377063 | 0 | 0 |
| 217847 | Serpina10 | 0.001328324 | 0.002565155 | 0.007206187 | 0.007802097 | 0.004874004 | 0 | 0 |
| 22351 | Vill | −0.00088276 | −0.00413782 | −0.00402831 | −0.00543647 | −0.00362318 | 0 | 0 |
| 29810 | Bag3 | 0.001772366 | 0.001612166 | 0.003542813 | 0.003909022 | 0.000171953 | 0 | 0 |
| 69861 | 2010003K11Rik | −0.00314845 | −0.00256501 | −0.00267891 | −0.00388259 | −0.00320474 | 0 | 0 |
| 15446 | Hpgd | −0.00264213 | −0.00123859 | −0.00033329 | 0.000594315 | 0.000844166 | 0 | 0 |
| 12799 | Cnp | 0.001303368 | 0.003160599 | 0.003041835 | 0.00514567 | 0.003768289 | 0 | 0 |
| 57437 | Golga7 | 0.001436272 | 0.000850956 | 0.000409907 | 0.000375902 | −0.00047768 | 0 | 0 |
| 235587 | Parp3 | 0.002119161 | 0.001010171 | 0.004864256 | 0.004493678 | 0.003838929 | 0 | 0 |
| 20661 | Sort1 | −0.00249055 | −0.00220303 | −0.00567416 | −0.00580875 | −0.00449366 | 0 | 0 |
| 234267 | Gpm6a | −0.00065479 | −0.00418265 | −0.00098383 | −3.89E−05 | −0.00106284 | 0 | 0 |
| 321018 | Serpina4-ps1 | 0 | −0.00539592 | 0 | 0 | 0 | 0 | 0.730941891 |
| 67708 | Pcnx4 | 0.000886505 | 0.003359187 | 0.000529214 | 0.00130452 | 0.000341525 | 0 | 0 |
| 106042 | Prickle1 | 8.06E−05 | 0.001847431 | 0.00111786 | 0.003105719 | 0.002995441 | 0 | 0 |
| 212943 | Tent5a | −0.00102709 | −0.00202167 | −0.0063729 | −0.00445699 | −0.00316538 | 0 | 0 |
| 18205 | Ntf3 | −0.00071723 | 0.005643016 | 0.00080084 | −0.00151776 | −0.00287103 | 0 | −0.32415892 |
| 20249 | Scd1 | 0.000684069 | 0.004925365 | 0.006264302 | 0.000225092 | 0.000972938 | 0 | −0.10596803 |
| 76454 | Fbxo31 | −0.00123826 | 0.000208474 | −0.00194789 | −0.00471663 | −0.00179502 | 0 | 0 |
| 17888 | Myh6 | −0.0007344 | −0.00010413 | 0.003113192 | 0.001288291 | −0.00072318 | 0 | 0 |
| 67564 | Tmem35a | −0.00137913 | −0.00251384 | −0.00232767 | −0.00068797 | −0.00069708 | 0 | 0 |
| 246278 | Cd207 | −0.00037964 | 0.00036521 | −0.00387832 | −0.00472388 | −0.00423906 | 0 | 0.281549313 |
| 71761 | Amdhd1 | −0.00156662 | −0.00616418 | 0.002039858 | −0.00069827 | −0.00105417 | 0 | 0 |
| 216233 | Sccs2 | −0.00022029 | −0.00492601 | 0.002482075 | 0.001785023 | 0.000711252 | −0.5947847 | −0.10002337 |
| 22335 | Vdac3 | 0.001323426 | 0.003139119 | 0.002294289 | 0.001718486 | 0.002458588 | 0 | 0 |
| 243529 | H1f10 | −0.0024752 | −0.00391159 | −0.00320032 | −0.00440327 | −0.00274197 | 0 | 0 |
| 12314 | Calm2 | 0.001715464 | 0.001976 | 0.000885504 | 0.001008979 | 0.000757137 | 0 | 0 |
| 66234 | Msmo1 | 0.0016032 | 0.000474108 | 0.00256576 | 0.003875778 | 0.002308044 | 0 | 0.003594303 |
| 14264 | Fmod | −0.00216329 | −0.00262619 | −0.00249739 | −0.00107804 | 0.000231988 | 0 | 0 |
| 104086 | Cyp27a1 | −0.00032016 | −0.00261831 | −0.00354615 | −0.00421213 | −0.00378671 | 0 | 0 |
| 67693 | Hypk | 0.00141435 | 0.002740288 | 0.002491234 | 0.001639696 | 0.002158432 | −0.29455101 | 0 |
| 71703 | Armcx3 | −0.00176512 | −0.00475494 | −0.00421262 | −0.00382837 | −0.00338187 | 0 | 0 |
| 15168 | Hcn3 | 0.0012518 | 0.004139119 | 0.005134319 | 0.005970114 | 0.005876198 | 0 | 0 |
| 67375 | Qprt | 0.000298739 | −0.00440703 | −0.00178516 | 0.000665055 | 0.001506728 | 0 | 0 |
| 214663 | Slc25629 | −0.0021902 | −0.00565001 | −0.00714849 | −0.00832115 | −0.0056507 | 0 | 0.057724885 |
| 79043 | Spsb3 | 0.00094097 | 0.00426933 | 0.005497959 | 0.004494983 | 0.003502228 | 0 | −0.00766007 |
| 16870 | Lhx2 | 0.001466572 | 0.003354707 | 0.002179088 | 0.003146044 | 0.002922998 | 0 | 0 |
| 114664 | Hsd17b11 | −0.00148846 | −0.00356825 | −0.00376176 | −0.00364856 | −0.0014718 | 0 | 0.063298573 |
| 433022 | Plcxd2 | −0.00055527 | −0.00162777 | 0.004490912 | 0.001523775 | 0.001738993 | 0 | 0 |
| 108030 | Lin7a | −0.00098442 | −0.00288494 | −0.0009749 | −0.00138892 | −0.00205837 | 0 | 0 |
| 240697 | Mcmdc2 | −0.00033427 | −0.00584581 | −0.00595135 | −0.00561993 | −0.00308582 | 0 | 0 |
| 72016 | Tedc2 | −0.00119962 | −0.00549271 | −0.00521428 | −0.00520727 | −0.00251395 | 0 | 0 |
| 18030 | Nfil3 | 0.001265002 | 0.003882736 | 0.001025118 | 0.005450811 | 0.002118148 | 0 | 0 |
| 66990 | Tmem134 | −0.00064507 | −0.00133709 | −0.00062874 | −0.00028339 | −0.0013501 | 0 | 0 |
| 68283 | 9530077C05Rik | 0.000736696 | 0.002823175 | 0.002542303 | 0.002425989 | −0.00021117 | 0 | 0 |
| 114142 | Foxp2 | −9.82E−05 | −0.00526714 | −0.00383049 | −0.00550919 | −0.00206877 | 0 | 0 |
| 78514 | Arhgap10 | 0.001278036 | 0.001877581 | 0.002487413 | 0.000790617 | 0.001997803 | 0 | −0.09160461 |
| 14869 | Gstp2 | 0 | −0.00316608 | 0 | 0 | 0 | 0 | 0 |
| 110382 | C8b | −0.00220633 | −0.00195852 | 0.000258505 | 0.000630384 | −0.0013773 | 0 | 0 |
| 65473 | Ctrb1 | −0.00070211 | −0.00342482 | 0.001866645 | 0.001798871 | 0 | 0 | 0 |
| 13122 | Cyp7a1 | 0.000783282 | 0.000657547 | −0.00122552 | −0.00269284 | −0.00188141 | 0 | −0.06680018 |
| 102545 | Cmtm7 | 3.35E−05 | −0.00382587 | −0.00078823 | 0.000621373 | −0.00220206 | 0 | 0 |
| 56277 | Tmem45a | −0.00020043 | −0.00321535 | −0.00185609 | −0.00188425 | −0.0011936 | 0 | 0 |
| 11835 | Ar | 0.003442426 | 0.002678543 | 0.001975532 | 0.002557018 | 0.003293783 | 0 | 0 |
| 16414 | Itgb2 | 0.003030949 | 0.004281795 | 0.004001098 | 0.006795075 | 0.00535698 | 0 | 0 |
| 56348 | Hsd17b12 | −0.00199636 | −0.0031612 | −0.00290515 | −0.00283252 | −0.00232971 | 0 | 0 |
| 72503 | 2610507B11Rik | −0.00134877 | −0.00195134 | −0.00525618 | −0.00492592 | −0.00303839 | 0 | 0 |
| 105245 | Txndc5 | 0.001622672 | 0.0040374 | 0.004136303 | 0.00350673 | 0.002876625 | 0 | 0 |
| 12986 | Csf3r | 0.003812166 | 0.00312229 | 0.006552784 | 0.008838054 | 0.005650189 | 0 | 0 |
| 140781 | Myh7 | −0.00048096 | −0.00190716 | −0.00205704 | −0.00043577 | −0.0012319 | 0 | 0 |
| 94109 | Csmd1 | 0.003339925 | 0 | 0.002754021 | 0.004528255 | 0.003282848 | 0 | 0 |
| 83925 | Trps1 | 0.001227783 | 0.004732178 | 0.005828114 | 0.005655812 | 0.004308383 | 0 | 0 |
| 69550 | Bst2 | 0.001182512 | −0.00068886 | −3.75E−05 | −0.00049193 | 5.29E−05 | 0 | 0 |
| 20750 | Spp1 | 0.002600358 | 0.000521086 | 0.006008283 | 0.002384106 | −0.00168279 | 0 | 0 |
| 170829 | Tram2 | −0.00058092 | −0.00229938 | −0.00284238 | −0.00393837 | −0.00193832 | 0 | 0 |
| 22190 | Ubc | −0.00106588 | −0.00237856 | −0.00486862 | −0.00419038 | −0.00354697 | 0 | 0 |
| 15205 | Hes1 | 0.001227984 | 0.000289677 | 0.001532806 | 0.001272151 | 0.000285013 | 0 | 0 |
| 227157 | Mpp4 | 0.001463586 | 0.005080331 | 0.006882142 | 0.001629287 | 0.002491318 | 0 | −0.31205601 |
| 64297 | Gprc5b | −0.00088236 | −0.00395759 | −0.00267434 | −0.00210013 | −0.00143539 | 0 | 0 |
| 98376 | Gorab | −0.00020526 | −0.00082481 | −0.00161508 | −0.00205368 | −0.00076451 | 0 | 0 |
| 14675 | Gna14 | 0.001604156 | 0.003688853 | 0.003799082 | 0.003819937 | 0.005204704 | 0 | −0.17124871 |
| 21859 | Timp3 | 0.002475178 | 0.002032297 | −0.00023613 | 0.002303899 | 0.003419098 | 0 | 0 |
| 21847 | Klf10 | 0.000370091 | 0.005160779 | −0.00057604 | 0.001380359 | −0.00173599 | 0 | 0 |
| 21391 | Tbxas1 | 0.000790096 | −0.00393011 | −0.0001483 | 0.000239448 | 0.000595557 | 0 | 0 |
| 246102 | Rttn | 0.000302583 | −0.0023034 | 0.000353848 | 0.001413017 | 0.001050707 | 0 | 0 |
| 72043 | Sulf2 | −0.00060944 | −0.00084815 | −5.19E−05 | 7.56E−06 | 2.98E−05 | 0 | 0 |
| 53612 | Vti1b | −0.00153774 | 0.000782548 | 0.001211571 | 0.001136712 | −0.00049782 | 0 | 0 |
| 235674 | Acaa1b | −0.00248766 | −0.00288511 | −0.00365312 | −0.00199128 | 0 | 0 | 0.064748623 |
| 19899 | Rpl18 | 2.14E−05 | 0.002220992 | 0.000462922 | 0.001280307 | 0.000839101 | 0 | 0 |
| 16483 | Kap | 0 | 0 | 0 | 0 | 0 | 0 | 0 |
| 55807 | Scamp5 | −0.00055555 | −0.00353021 | −0.00151707 | −0.00191597 | −0.00064077 | 0 | 0 |
| 276950 | Slfn8 | 0.003105012 | 0.004795612 | 0.008276894 | 0.011227665 | 0.00678502 | 0 | 0 |
| 14686 | Gnat2 | −0.00142744 | −0.00189764 | −0.00171219 | −0.00604093 | −0.00252576 | 0 | −0.04110814 |
| 54371 | Chst2 | −0.00193022 | −0.00306599 | 0.000201649 | 0.000497345 | −0.0001245 | 0 | 0 |
| 20525 | Slc2a1 | 7.17E−05 | −0.00099298 | −0.00052303 | −0.00168794 | −0.00146703 | 0 | 0 |
| 12827 | Col4a2 | −0.0028133 | −0.00262367 | −0.00353769 | −0.00449667 | −0.00464442 | 0 | 0 |
| 239447 | Colec10 | −0.00140751 | −0.00309695 | −0.00359729 | −0.00225429 | −0.00199406 | 0 | 0 |
| 66686 | Dcbld1 | −0.00138618 | −0.0023637 | −0.00268194 | −0.00271341 | 0.001170307 | 0 | −0.07294719 |
| 66970 | Ssbp2 | 0.001089961 | −0.00229212 | 0.002064657 | 0.002277368 | 0.001610935 | 0 | −0.02060664 |
| 320981 | Enpp6 | −0.00136316 | 0.001374254 | 1.98E−05 | 0.000590777 | −0.0008567 | 0 | 0 |
| 20104 | Rps6 | 0.000892168 | 0.000119555 | −0.00026136 | 0.00054435 | 0.001550838 | 0 | 0 |
| 76574 | Mfsd2a | −0.00150816 | −0.00198514 | −0.00016468 | 0.000765834 | 0.000557657 | 0 | 0 |
| 58193 | Rpl24 | 0.001448698 | −0.00105005 | −0.00226998 | −0.0019214 | −0.00124299 | 0 | 0 |
| 21956 | Tnnt2 | 0.002244451 | −0.00544096 | −0.00097496 | 0.001020384 | 0.001478286 | 0 | 0.140590164 |
| 15068 | Il18bp | 0.000120046 | 0.002939082 | −0.00218627 | −0.00321859 | −0.00108539 | 0 | 0 |
| 214191 | Ttc24 | 0 | −0.00358441 | 0 | 0 | −0.00052025 | 0 | 0 |
| 105428 | Fam149b | −0.0010694 | −0.00317999 | −0.00260907 | −0.00219825 | −0.00059544 | 0 | 0 |
| 240776 | Kcnt2 | 0.00261642 | 0.002107028 | 0.003626969 | 0.002541485 | 0.002806734 | 0 | 0 |
| 23882 | Gadd45g | −0.00041716 | −0.00128276 | 0.000121113 | 0.00047662 | −0.00278084 | −0.23211885 | 0 |
| 14120 | Fbp2 | 0.003585245 | 0.003074793 | 0.004072788 | 0.00703996 | 0.005277654 | 0 | 0 |
| 17288 | Mep1b | 0.001280498 | 0.003309471 | 0.002164222 | 0.002219056 | 0.002624305 | 0 | 0 |
| 245038 | Dclk3 | −0.00015641 | 0.00111095 | −0.00135027 | −0.00061613 | −0.00140415 | 0 | 0 |
| 11980 | Atp8a1 | 0.001780827 | 0.001758631 | −0.00057389 | 0.002395891 | 0.002260475 | 0 | 0 |
| 55320 | Dbn1 | −0.00095549 | −0.00533209 | 0.000268182 | −0.00019684 | 0.00037287 | 0 | 0 |
| 12349 | Car2 | −0.00075362 | −0.00548802 | −0.00077765 | 0.001371555 | −0.0022125 | 0 | 0 |
| 14219 | Con2 | 3.91E−05 | 0.002796587 | −0.00180968 | −0.00038093 | 0.001665044 | 0 | 0.243784444 |
| 226654 | Tstd1 | 0.000219736 | 0.002555835 | 0.001901537 | 0.000856448 | 0.002038114 | 0 | 0 |
| 109791 | Clps | 0.000177761 | −0.00173497 | −0.00127834 | −0.00073716 | −0.00061388 | 0 | 0 |
| 70082 | Lysmd2 | −0.00187032 | −0.003489 | −0.00336146 | −0.00463813 | −0.00187564 | 0 | 0 |
| 12111 | Bgn | 0.001905285 | −0.00045769 | 0.002942986 | 0.001184703 | 0.001529948 | 0 | 0 |
| 22283 | Ush2a | 0.001366491 | 0.004434837 | 0.002140375 | 0.003017113 | 0.003811847 | 0 | 0 |
| 235469 | Zfp280d | 0.001413012 | 0.001565641 | −0.00059248 | 0.000619781 | 0.000656536 | 0 | 0 |
| 108098 | Med21 | −0.00166546 | −0.00448637 | −0.0038656 | −0.00455923 | −0.00124893 | 0 | 0 |
| 16518 | Kcnj2 | 0.000165552 | −0.00366485 | 4.56E−05 | −2.82E−05 | −2.79E−05 | 0 | 0.038449034 |
| 79196 | Osbpl5 | −0.0015686 | −0.0024123 | −0.00137097 | −0.0035666 | −0.0030835 | 0 | 0.066048824 |
| 245269 | Nim1k | −0.00215822 | −0.00289724 | −0.00212475 | −0.00289375 | −0.00231814 | 0 | 0 |
| 17960 | Nat1 | 0.001556955 | 0.003043894 | 0.004545641 | 0.0041004 | 0 | 0 | 0 |
| 20715 | Serpina3g | −0.00067612 | −0.00531327 | −0.00059861 | −0.0029203 | 0 | 0 | 0 |
| 13835 | Epha1 | −0.0010562 | −0.00042215 | −0.00045046 | −0.00029941 | −0.00186618 | 0 | 0 |
| 59020 | Pdzk1 | 0.002270321 | 0.001086711 | 0.000819486 | 0.001744175 | 0.000948917 | 0 | 0 |
| 225215 | Rsl24d1 | 0.00141554 | 0.001645374 | 0.002357574 | 0.003081448 | 0.002707463 | 0 | 0 |
| 236900 | Pdk3 | 0.000150296 | 0.001870516 | 0.002137326 | 0.000995845 | 0.001722708 | 0 | 0 |
| 75530 | Lyrm7 | −0.00035922 | 0.003699145 | 7.39E−06 | −0.00089985 | 0.000716675 | 0 | 0.017109515 |
| 14533 | Bloc1s1 | 0.001553701 | 0.004010138 | 0.003235918 | 0.001862387 | 0.002450901 | 0 | 0 |
| 20607 | Sstr3 | −0.00152461 | 0 | 0 | −0.00476229 | −0.0021974 | 0 | 0 |
| 12554 | Cdh13 | 0.000916979 | 0.002136776 | −0.0021606 | 0.002297354 | 0.002971133 | 0 | 0 |
| 80794 | Cblc | 0.000348509 | 0.004926615 | −0.00014109 | 0.002441398 | 0.002023248 | 0 | 0 |
| 19261 | Sirpa | 0.001649131 | 0.003409903 | −0.00016323 | 0.001226478 | 0.001707351 | 0 | 0.060833551 |
| 72027 | Slc39a4 | −0.00058314 | −0.00299094 | 0.000875258 | 0.002474789 | 0.000622783 | 0 | −0.26320787 |
| 69938 | Scrn1 | 0.00045783 | 0.002695632 | −0.0008121 | 0.002483975 | 0.002453852 | 0 | 0 |
| 236293 | Slc22a29 | 0 | −0.00017843 | 0 | 0 | 0 | 0 | 0 |
| 12259 | C1qa | 0.003929245 | 0.002811729 | 0.003102987 | 0.002835506 | 0.002509308 | 0 | 0 |
| 237256 | Zc3h12d | 0.002425924 | 0.004947082 | 0.002830777 | 0.002442651 | 0.003360991 | 0 | −0.06819638 |
| 14313 | Fst | 0.001564896 | 2.47E−05 | 0.000965267 | −0.00024879 | 0.001893898 | 0 | 0 |
| 54385 | Cyp4f14 | −0.00096161 | 0.000323609 | 0.00024988 | 0.000619989 | 0.001095884 | 0 | 0 |
| 218121 | Mbcat1 | −0.00076897 | −0.00451998 | −0.00374066 | −0.00362772 | −0.00087022 | 0 | 0 |
| 16952 | Anxa1 | 0.002402212 | 0.003756795 | 0.002359975 | 0.002561622 | 0.001923504 | 0 | 0 |
| 66689 | Klhl28 | −0.00174565 | −0.00247375 | −0.00426343 | −0.00448271 | −0.00172841 | 0 | 0 |
| 245404 | Dcaf12l1 | −0.00029375 | −0.0049926 | −0.00361908 | −0.00500157 | −0.0018862 | 0 | 0 |
| 232334 | Vgll4 | −0.00090103 | 0.000768368 | −0.00093583 | −0.0004646 | −0.00014821 | 0 | 0 |
| 225289 | AW554918 | 0.000682406 | −0.00117739 | −0.00106469 | 0.000619192 | 0.000484817 | 0 | 0 |
| 16987 | Lss | 0.002079123 | 0.005255036 | 0.004797115 | 0.00359806 | 0.004841859 | 0 | 0 |
| 54403 | Slc4a4 | 0.001706171 | 0.001871994 | −0.00085744 | 0.000376728 | −0.00034515 | 0 | 0 |
| 24057 | Sh3yl1 | 0.00114927 | 0.003868532 | 0.001972984 | 0.004481775 | 0.004404478 | 0 | 0 |
| 269799 | Clec4a1 | −6.38E−05 | −0.00711511 | −0.00650962 | −0.00570895 | −0.00312726 | 0 | 0 |
| 14368 | Fzd6 | −0.00021853 | −0.00258808 | −0.00033928 | −0.00184072 | −0.00108964 | 0 | 0 |
| 15803 | Lbp | 0.000971161 | 0.003612349 | −0.0017275 | 0.002853073 | 0.003419917 | 0 | 0 |
| 20342 | Selenbp2 | 0 | −0.00171461 | −0.0014094 | 0 | 0 | 0 | 0.099827706 |
| 17540 | Irag1 | 0.000939548 | 0.002721257 | 0.003400743 | 0.000636103 | 0.000818021 | 0 | −0.03779462 |
| 28015 | Polr2m | 0.001025848 | 0.003175053 | 0.002746053 | 0.002950078 | 0.002518279 | 0 | 0 |
| 212032 | Hk3 | −0.00069331 | 5.06E−06 | −0.0051709 | −0.00512156 | −0.00389448 | 0 | 0 |
| 18162 | Npr3 | 0.001151977 | −0.00365859 | 0.000170958 | 0.002313832 | 0.001030661 | 0 | 0 |
| 72993 | Appl1 | −0.00088545 | −0.00182246 | −0.00138778 | −0.00129294 | −0.00243445 | 0 | 0 |
| 20266 | Scn1b | 0.001701304 | 0.003452323 | 0.005073557 | 0.005527253 | 0.003243113 | 0 | 0 |
| 78177 | Ninl | 0.001229357 | −0.00089174 | −6.24E−05 | −0.00051168 | −0.00019065 | 0 | 0 |
| 106757 | Catsperd | −0.00027624 | 3.16E−05 | 0.00033644 | −8.01E−05 | −0.00062979 | 0 | −0.057031 |
| 14062 | F2r | −3.44E−05 | 0.003020221 | 0.002647673 | 0.001462362 | −0.00042899 | 0 | 0 |
| 233744 | Spon1 | 0.000469653 | 0.000460891 | 0.001991546 | 0.003045398 | 0.000940643 | 0 | 0 |
| 12830 | Col4a5 | −0.00401107 | −9.27E−05 | −0.00037322 | −0.00401028 | −0.00302142 | 0 | 0.303925159 |
| 19041 | Ppl | −0.00052081 | −0.00393875 | −0.00642007 | −0.00869375 | −0.0046307 | 0 | 0 |
| 52033 | Pbk | 0.000651131 | −0.00361999 | 0.001866117 | 0.002411915 | 0.001862974 | 0 | 0 |
| 108150 | Galnt7 | 2.61E−05 | −0.00376654 | −0.00018628 | −0.00286608 | −0.00159157 | 0 | 0 |
| 103967 | Dnm3 | 0.001651113 | 0.005122019 | 0.005392879 | 0.005592693 | 0.004170799 | 0 | 0 |
| 70012 | Cep85 | 0.001490974 | 0.002512745 | 0.003948449 | 0.003963351 | 0.001914131 | 0 | −0.06416746 |
| 246133 | Kcne2 | −0.00197374 | −0.00123733 | −0.00357119 | −0.00492105 | −0.00353097 | 0 | 0 |
| 12305 | Ddr1 | −0.00140685 | −0.00473324 | −0.00312606 | −0.00353632 | −0.0035955 | 0 | 0 |
| 226781 | Slc30a10 | 0.000600915 | 0.00348057 | 0.00183111 | −0.00226255 | −0.00119561 | 0 | 0 |
| 14231 | Fkbp7 | −0.0002424 | 0.005536464 | 0.003292177 | 0.00455138 | 0.002463837 | 0 | 0 |
| 12160 | Bmp5 | −0.00111302 | −0.00081515 | 7.50E−05 | 0.000581215 | −0.00067293 | 0 | 0 |
| 29818 | Hspb7 | 0.000440012 | −0.00282765 | −0.00422303 | −0.00539601 | −0.00504927 | 0 | 0 |
| 20400 | Sh2d1a | 0.001483564 | 0.001887016 | 0.0034164 | 0.001762456 | 0.002990341 | 0 | 0 |
| 68603 | Pmvk | 0.000946067 | 0.003285539 | −0.00014336 | 0.001061895 | 0.002752679 | 0 | 0.117062688 |
| 16880 | Lifr | 0.001244955 | 0.004836504 | 0.003110659 | 0.005842924 | 0.004074354 | 0 | 0 |
| 17087 | Ly96 | −0.0020623 | −0.00164813 | 0.00327486 | 0.0034855 | −0.00354245 | 0 | 0 |
| 16431 | Itm2a | −0.00332087 | 0.001272528 | −0.00739927 | −0.00747439 | −0.00217685 | 0 | 0 |
| 22630 | Ywhaq | −0.00036092 | 6.91E−05 | 0.00073972 | −0.00030706 | −0.00104789 | 0 | −0.0387156 |
| 14580 | Gfap | 0.002402343 | −0.00029285 | 0.005474075 | 0.005111838 | 0.006440595 | 0 | 0 |
| 574402 | Gpr17 | −0.00219961 | 0.00381102 | −0.0041489 | −0.00353688 | −0.00603416 | 0 | 0.011743356 |
| 12494 | Cd38 | −0.00222116 | 0.001409259 | 0.00520336 | 0.0084809 | −0.00289747 | 0 | −0.01454591 |
| 93710 | Pcdhga2 | 0.002597057 | 0 | 0.005532945 | 0.00455127 | 0.004004188 | 0 | 0 |
| 20787 | Srebf1 | 0.001251493 | 0.000354907 | 0.000557859 | 0.001365193 | 0.001942029 | 0 | 0 |
| 105243 | Slc9a3 | 0 | 0 | 0 | 0 | 0.000295931 | 0 | 0 |
| 64706 | Scube1 | −0.00110371 | 0 | −0.00389106 | −0.00466077 | −0.0021019 | 0 | 0 |
| 93880 | Pcdhb9 | 0.002855799 | 0 | 0.010971014 | 0.009388718 | 0 | 0 | 0 |
| 11688 | Alox8 | 0.002082711 | −0.00146337 | 0.002633662 | 0.004987619 | 0.003545606 | 0 | 0 |
| 19277 | Ptpro | −0.00118921 | −0.00375972 | −0.00231442 | −0.00431884 | −0.00289212 | 0 | 0 |
| 381823 | Apold1 | 0.001265518 | 0.002102007 | 0.001935082 | 0.003098412 | −9.25E−05 | 0 | 0 |
| 19143 | St14 | 0.002091119 | 0.000739567 | −0.00094238 | −0.00033229 | 0.000776202 | 0 | 0 |
| 29870 | Gtse1 | 0.000259724 | 0.000243778 | 0.0014118 | 0.000326887 | 0.000737019 | 0 | 0 |
| 68895 | Rasl11a | −0.00050631 | −0.00047785 | 0.000895352 | 0.00040282 | 0.001029568 | 0 | 0 |
| 12877 | Cpeb1 | 0.004343524 | −0.00138371 | 0.00292034 | 0.006362015 | 0.004194659 | 0 | 0 |
| 13106 | Cyp2e1 | −0.00112241 | 0.001075184 | 0.005354815 | 0.003898781 | 0.003423649 | 0 | 0 |
| 12814 | Col11a1 | −0.00397797 | 0 | −0.00109703 | −0.00480243 | −0.00503027 | 0 | 0 |
| 52712 | Zkscan6 | −0.00132096 | −0.00136389 | −0.00431864 | −0.00479016 | −0.00357894 | 0 | 0 |
| 11475 | Acta2 | −0.00042575 | 0.002754875 | 0.003212 | 0.002512574 | −0.00056575 | 0 | −0.08203894 |
| 21823 | Th | 0.000770983 | −0.00067462 | 0.005558765 | 0.007063758 | 0.00157848 | 0 | 0 |
| 16149 | Cd74 | 0.00357548 | 0.002034449 | −0.00162227 | 0.003614512 | 0.001843851 | 0 | 0.283301035 |
| 78781 | Zc3hav1 | 0.001808945 | 0.001396056 | 0.003194763 | 0.004911425 | 0.00240084 | 0 | 0 |
| 16012 | Igfbp6 | −0.00098089 | 0.000643845 | −0.00080569 | −0.0003603 | 0.000197827 | 0 | −0.00524183 |
| 100039795 | Ildr2 | 0.001669079 | 0.004207794 | 0.006475783 | 0.007806591 | 0.006579874 | 0 | −0.39726303 |
| 12807 | Hps3 | −0.00151143 | −0.0008176 | −0.00460788 | −0.00380745 | −0.00350645 | 0 | 0 |
| 93873 | Pcdhb2 | 0.000927614 | 0 | 0 | 0.002426338 | 0.003960353 | 0 | 0 |
| 20322 | Sord | 0.002579062 | 0.001869047 | 0.004960449 | 0.004306437 | 0.00280365 | 0 | 0 |
| 59054 | Mrps30 | 0.002192336 | −2.97E−05 | 0.002725463 | 0.002997648 | 0.003390089 | 0 | 0 |
| 238271 | Kcnh5 | −0.00181568 | 0.000173017 | −0.00313748 | −0.00370563 | −0.002639 | 0 | 0 |
| 19039 | Lgals3bp | 0.00292783 | 0.002614199 | 0.004704595 | 0.005435126 | 0.000449772 | 0 | 0 |
| 246104 | Rhbdl3 | −0.00192877 | −0.0007226 | −0.00446859 | −0.00406232 | 0.001226055 | 0 | 0 |
| 65116 | Prrg2 | −0.00066873 | 4.70E−05 | −0.00164862 | −0.00308906 | −0.00108416 | 0 | 0 |
| 102614 | Rpp25 | 0.000840756 | −0.00048808 | 0.000267526 | 0.001525437 | 0.001529434 | 0 | 0 |
| 69926 | Dnah17 | −0.00074818 | −0.00292249 | −0.0029764 | −0.00370188 | −0.00287331 | 0 | 0.126331606 |
| 17357 | Marcksl1 | −0.00210047 | −0.00180474 | −0.00624569 | −0.00596562 | −0.00228293 | 0 | 0 |
| 319480 | Itga11 | −0.00176808 | −0.00025433 | 0.001135867 | −0.00219807 | −0.0018087 | 0 | 0 |
| 14412 | Slc6a13 | −0.00226866 | −0.00014981 | 0.000780654 | −0.00226091 | −0.00120394 | 0 | 0 |
| 73914 | Irak3 | 0.003773698 | 0.001691003 | 0.003904344 | 0.006197679 | 0.001729557 | 0 | 0 |
| 24088 | Tlr2 | 0.002370298 | −0.00141997 | 0.005581214 | 0.004936612 | 0.003225547 | 0 | 0 |
| 54132 | Pdlim1 | 0.00183661 | 0.002564612 | 0.005807511 | 0.006582053 | 0.004704815 | −0.01019887 | −0.16591235 |
| 208890 | Slc26a7 | −0.0010857 | 0 | −0.00213647 | −0.00622921 | −0.00312984 | 0 | 0 |
| 11865 | Arntl | −0.00078109 | 0.003712446 | 0.003022788 | 0.001596766 | 0.000432591 | 0 | 0 |
| 237928 | Phospho1 | 0.00085452 | 0.002387422 | 0.000456446 | 0.000584837 | −0.00066516 | −0.36050685 | 0 |
| 18542 | Pcolce | −0.00144225 | 0.000789979 | 0.001034223 | 5.92E−05 | −0.00067663 | 0 | 0 |
| 114249 | Npnt | −0.00172435 | 0.000313389 | 0.000283881 | 1.81E−05 | 0.000732129 | 0 | 0 |
| 55990 | Fmo2 | 0.00335021 | 0.001828103 | 0.00481092 | 0.005892439 | 0.003504544 | 0 | 0 |
| 11898 | Ass1 | 0.001366986 | −0.00047197 | 0.00118434 | −0.00145077 | −0.0012771 | 0 | 0 |
| 108115 | Slcc4a1 | 0.000370944 | 0.001955367 | −0.00385955 | −0.00155471 | −0.00151034 | 0 | 0 |
| 217779 | Lysmd1 | −0.00172429 | 0.000812937 | −0.0033584 | −0.00570406 | −0.00458187 | 0 | 0 |
| 329628 | Fat4 | −0.00216155 | 0.002952903 | −0.00273875 | −0.00408004 | −0.0039112 | 0 | 0 |
| 268527 | Greb1 | −9.95E−05 | 0.000231026 | 0.002560069 | 0.002400269 | 0.000591091 | 0 | 0 |
| 11438 | Chrna4 | −0.00063551 | −0.0028833 | −0.00188945 | −0.00118875 | −0.00093321 | 0 | 0.075214718 |
| 18682 | Phkg1 | −0.0022821 | 0.00088526 | −0.00472194 | −0.00452639 | −0.00277044 | 0 | 0 |
| 13386 | Dlk1 | 0.001349581 | 0 | 0.001913397 | 0.003905339 | 0.003796195 | 0 | 0 |
| 22063 | Trpc1 | 0.00404368 | 0.002081701 | 0.006662059 | 0.007707621 | 0.005717645 | 0 | 0 |
| 93840 | Vangl2 | −0.00292068 | −0.00261284 | −0.00208521 | −0.00515539 | −0.00509145 | 0 | 0 |
| 66898 | Baiap2l1 | −0.00022965 | −0.00117679 | 0.002306415 | 0.001608926 | 8.21E−05 | 0 | 0 |
| 80892 | Zfhx4 | 0.001118927 | −0.00194289 | 0.001113546 | 0.000334549 | 0.001544831 | 0 | 0.074663697 |
| 269437 | Plch1 | −0.00116537 | 0.001620098 | −0.00186454 | −0.00289652 | −0.00110764 | 0 | 0 |
| 108800 | Ston2 | −0.00162148 | 0.000465555 | −5.48E−05 | −0.00082487 | −0.00220292 | 0 | 0 |
| 19739 | Rgs9 | −0.00198231 | 0.002395795 | 0.000126881 | 0.000141448 | −1.23E−05 | 0 | 0 |
| 627626 | Ptchd4 | 0 | 0 | −0.00082834 | 0 | −0.00119109 | 0 | 0 |
| 233836 | Slc5a11 | 0 | 0 | 0 | 0 | −0.00142547 | 0 | 0 |
| 66089 | Rmnd5b | −0.00111206 | −0.00078044 | −0.00045243 | 5.54E−05 | 0.001137795 | 0 | 0 |
| 17349 | Mlf1 | −0.00281961 | 6.22E−05 | −0.00095536 | −0.00326911 | −0.00379897 | 0 | 0 |
| 382252 | Bciaf3 | −0.00016077 | −0.0013453 | −0.00042386 | 0.000209139 | 2.35E−05 | 0 | 0 |
| 78267 | Klhdc8b | −0.00183675 | 0.00020481 | −0.00332096 | −0.00336581 | −0.0037147 | 0 | 0 |
| 20776 | Tmie | 0.000853619 | 0.00204331 | 0.004342058 | 0.004665664 | 0.00202569 | 0 | 0 |
| 105440 | Kctd9 | −0.00101514 | −0.00051557 | −0.00197527 | −0.00244799 | 0.000124518 | 0 | 0 |
| 231999 | Plekha8 | −0.0010573 | −0.00088838 | −0.00316495 | −0.00284646 | −0.00257577 | 0 | 0 |
| 52463 | Tet1 | −0.00363327 | −0.00016401 | −0.00568658 | −0.0027566 | −0.0043751 | 0 | 0 |
| 12010 | B2m | 0.00335584 | 0.002472341 | 0.003929152 | 0.005113591 | 0.002593728 | 0 | 0 |
| 50917 | Galns | 0.001037768 | 0.000237262 | 0.003275446 | 0.002734045 | 0.001397469 | 0 | 0 |
| 18217 | Ntsr2 | 0.000853578 | 0 | 0.002677351 | 0.001944859 | 0.002410605 | 0 | 0 |
| 54357 | Epb41l4b | 0.000859303 | −0.00016769 | 0.003753162 | 0.003563901 | 0.001296187 | 0 | 0 |
| 239337 | Adamts12 | −0.00322391 | 0 | −0.00289055 | −0.00328032 | −0.0038202 | 0 | 0 |
| 110959 | Nudt19 | 0.000875524 | −0.00016511 | 0.002747139 | 0.003925892 | 0.002057168 | 0 | 0.032142345 |
| 237213 | Glra2 | 0 | 0 | 0 | 0 | −0.00284044 | 0 | 0 |
| 24117 | Wif1 | 0.000993869 | 0.001808667 | 0.002636617 | 0.00155261 | 0.001208022 | 0 | 0 |
| 17932 | Myt1 | −0.00165217 | −0.00011497 | −0.0036308 | −0.00363831 | −0.00268542 | 0 | 0 |
| 15114 | Hap1 | 0.000586145 | −0.00226255 | −0.00014379 | 0.002053946 | 0.001761592 | 0 | 0.00142759 |
| 216578 | Papolg | −0.00117885 | 0.002160496 | −0.00195124 | −0.0022589 | −0.00061225 | 0 | 0 |
| 72852 | Mblac2 | 0.000678382 | −0.00398229 | −0.00104224 | −0.00124254 | 0.000442889 | 0 | 0.060765054 |
| 215387 | Ncaph | −0.00274672 | 0.003027951 | −0.00040547 | −0.00110267 | −5.58E−05 | 0 | 0 |
| 14706 | Gng4 | 0.000905278 | −0.00064888 | 0.002560863 | 0.000791633 | −0.00189014 | 0 | 0 |
| 22177 | Tyrobp | 0.003004462 | 0.00074507 | 0.005303477 | 0.004630337 | 0.001484363 | 0 | 0 |
| 19224 | Ptgs1 | 0.000504512 | −0.00188971 | −0.00106071 | 0.000991283 | 0.001460953 | 0 | 0 |
| 69786 | Tprkb | 5.94E−05 | 0.000646335 | 0.00095176 | 0.001067637 | 0.000773264 | 0 | 0 |
| 433323 | Sgpp2 | 0.002423679 | 0.000683642 | −8.75E−05 | 4.67E−05 | 0.001232743 | 0 | 0 |
| 210293 | Dock10 | −0.00252807 | −0.00123074 | −0.00576767 | −0.00504295 | −0.00421797 | 0 | 0 |
| 70918 | Nsun7 | −0.00071973 | −0.00091849 | −0.00332666 | −0.00468863 | −0.00396179 | 0 | 0 |
| 71897 | Lypd6b | 0.000706055 | −0.00057548 | −0.00199155 | −0.00058485 | −0.00172284 | 0 | 0 |
| 26949 | Vat1 | 0.002963995 | 0.00025254 | 0.002739072 | 0.003921957 | 0.005157145 | 0 | 0 |
| 108797 | Mex3b | −0.00246614 | 0.000303999 | −0.00219933 | −0.00376916 | −0.00345395 | 0 | 0 |
| 20055 | Rps16 | −0.00230814 | −0.00159586 | −0.00446427 | −0.00388267 | −0.00318099 | −0.0590503 | 0.026364462 |
| 52626 | Cdkn2aipnl | 0.001372278 | −0.00049767 | 0.000463521 | −0.00049731 | 0.000480672 | 0 | 0 |
| 13143 | Dapk2 | −0.0005294 | 0.000649379 | 0.00162751 | −0.00127527 | 0.002444261 | 0 | 0 |
| 75646 | Rai14 | 0.002816021 | 0.000660089 | 0.00844734 | 0.010287172 | 0.005405696 | 0 | 0 |
| 109229 | Fam118b | −0.00202312 | 0.00345477 | 0.00270117 | −0.00191986 | −0.00292506 | 0 | 0 |
| 12266 | C3 | 0.003865771 | 0.001382579 | 0.006309216 | 0.007533261 | 0.006665929 | 0 | 0 |
| 207596 | Thsd4 | −0.00020821 | 0.000713284 | 0.001612064 | 0.001469796 | 0.002020715 | 0 | 0 |
| 53324 | Nptx2 | −0.00193407 | −0.00097242 | 0.00275214 | −0.00307308 | 0.000116793 | 0 | 0 |
| 12426 | Cckbr | 0.000384631 | 0.000114161 | 0.000757092 | −0.00029987 | −0.00067045 | 0 | 0 |
| 12955 | Cryab | 0.001286589 | 0.004457564 | −0.00079435 | 0.002726187 | 0.00370244 | 0 | 0 |
| 380614 | Intu | 0.000784173 | −0.00187703 | −0.00237987 | −9.22E−05 | 0.001170666 | 0 | 0 |
| 226098 | Hectd2 | −0.00046281 | 0.000147121 | −0.00126048 | −0.00209047 | −0.00078104 | 0 | 0 |
| 23999 | Twf2 | −0.00248675 | 0.001412631 | −0.00235464 | −0.00261028 | −0.00364994 | 0 | 0 |
| 12380 | Cast | 0.001202862 | −0.00064734 | 0.00219834 | 0.002014261 | 0.000651094 | 0 | 0 |
| 93707 | Pcdhgc4 | −0.00127099 | 9.09E−05 | −0.00046535 | −0.00058916 | −0.00024913 | 0 | 0 |
| 72018 | Fundc1 | 0.000370448 | 0.000518603 | −1.42E−05 | 0.000314724 | −0.00054982 | 0 | 0 |
| 66606 | Lrrc57 | −0.00013796 | −0.00055668 | −0.00156547 | −0.00136607 | −0.00236711 | 0 | 0 |
| 26934 | Racgap1 | −0.00312975 | 0.001669917 | −0.00354451 | −0.00474451 | −0.00362395 | 0 | 0 |
| 83768 | Dpp7 | 0.000117075 | 0.001051302 | −0.00103363 | 6.08E−05 | −0.00099709 | 0 | −0.13919421 |
| 22325 | Vav2 | −0.00173148 | 0.000887742 | −0.00335357 | −0.0022691 | −0.00139196 | 0 | 0 |
| 73162 | Otud3 | −0.00165461 | 0.003097677 | −0.00153518 | −0.00049317 | −0.00241916 | 0 | 0 |
| 93887 | Pcdhb16 | 0.002469137 | 0.001461716 | 0.002629092 | 0.003157336 | 0.002619581 | 0 | 0 |
| 69297 | Lrrc46 | −0.00104934 | 0.000835319 | −0.00333125 | −0.00656602 | −0.00303119 | 0 | 0 |
| 22290 | Uty | 0.001596747 | 0.00224491 | −0.00011058 | 2.60E−05 | 0.000323506 | 0 | 0 |
| 269941 | Chsy1 | 0.001007122 | 0.001904067 | 0.000293975 | 0.001184998 | 0.0012543 | 0 | 0 |
| 12839 | Col9a1 | −0.00422518 | 0.000900241 | −0.00709376 | −0.01025596 | −0.00642265 | 0 | 0 |
| 223827 | Gxylt1 | 0.001072201 | −0.00157211 | 0.000635702 | −0.00015504 | 0.001402033 | 0 | 0 |
| 75600 | Calml4 | −0.00110648 | −0.00393373 | −0.00214848 | −0.00477922 | −0.00139925 | 0 | 0 |
| 59031 | Chst12 | −0.00020628 | 0.002425473 | 0.001679904 | 0.000557795 | 0.001170195 | 0 | 0 |
| 93891 | Pcdhb20 | 0.001334629 | −3.10E−05 | 0.003315 | 0.004836622 | 0.003830542 | 0 | 0 |
| 71918 | Zcchc24 | 0.000929538 | −0.00185777 | −0.0021981 | −0.00261558 | −0.0004511 | 0 | 0 |
| 58250 | Chst11 | −0.0012949 | 0 | −0.00135671 | −0.00281313 | −0.00242035 | 0 | 0 |
| 68770 | Phtf2 | 0.000813771 | −0.00168138 | 0.001506468 | 0.002527691 | 0.000387634 | 0 | 0 |
| 67405 | Nts | −0.00122355 | 0.000569421 | −0.00170854 | −0.00193468 | −0.00056023 | 0 | 0 |
| 381853 | Gipr | 0 | 0 | 0 | 0 | 0.00257175 | 0 | 0 |
| 231134 | Dok7 | 0.000767635 | 8.34E−06 | 0.001453325 | 0.001698256 | 0.001106413 | 0 | 0 |
| 13170 | Dbp | −0.00098174 | 0.003230054 | −0.00024789 | −0.00020699 | 0.000171152 | 0 | −0.06636802 |
| 63955 | Cables1 | −0.00091235 | −0.00063715 | −0.00345209 | −0.004424 | −0.00214116 | 0 | 0 |
| 14281 | Fos | 0.002078802 | −0.00038608 | 0.00439983 | 0.007021382 | 0.002742258 | 0 | 0 |
| 18125 | Nos1 | −0.00017604 | −0.00095997 | −0.00158121 | −0.00426009 | −0.00163263 | 0 | 0 |
| 78912 | Sp2 | 0.001068368 | −0.00101477 | 0.00100049 | 0.000405218 | 0.000891339 | 0 | 0 |
| 75563 | Dnali1 | −0.00179817 | −0.00022725 | −0.00564571 | −0.00495993 | −0.00196125 | 0 | 0 |
| 218442 | Serinc5 | −0.00202398 | −0.00027041 | −0.00440358 | −0.0059519 | −0.00309385 | 0 | 0 |
| 19201 | Pstpip2 | 0.001006244 | 0.000455141 | 0.001310267 | 0.000751098 | −0.00107554 | 0 | 0 |
| 66960 | Mlndy3 | −0.00083134 | −0.0002667 | −0.00268339 | −0.00281636 | −0.0018802 | 0 | 0 |
| 53600 | Tlmm23 | −0.00145849 | −0.000379 | −0.00128317 | −0.00120287 | −0.00096847 | 0 | 0 |
| 67477 | Abhd15 | −0.00105712 | −0.00099352 | −0.00145758 | −0.00178788 | −0.00040365 | 0 | 0 |
| 27277 | Golga5 | 0.000806813 | 0.000785318 | −6.89E−05 | 0.000703234 | 0.001022587 | 0 | 0 |
| 242702 | Myom3 | −2.08E−05 | −0.00244058 | 0.002673768 | 0.001080913 | 0.000788993 | 0 | 0 |
| 27207 | Rps11 | 0.00086132 | −8.81E−05 | −0.00158517 | −0.00138735 | 0.000523805 | 0 | 0 |
| 271127 | Adamts16 | −0.00280138 | −0.00206578 | −0.0021914 | −0.0065097 | −0.00363111 | 0 | 0 |
| 114585 | D17H6353E | −0.00149724 | −0.00151681 | −0.00037485 | −0.00166918 | −0.00161929 | 0 | 0 |
| 15901 | Id1 | 0.001713816 | −0.00031859 | 0.00117843 | 0.003561866 | 0.004277074 | 0 | 0 |
| 194268 | 9930104L06Rik | 0.00033481 | −0.00056564 | −0.00283193 | −0.00427705 | −0.00143184 | 0 | 0 |
| 319899 | Dock6 | −0.00053794 | −0.00145634 | −0.00287424 | −0.00386748 | −0.00241876 | 0 | 0 |
| 22371 | Vwf | 0.004475125 | 0.000477212 | 0.007867522 | 0.009317104 | 0.009471662 | 0 | 0 |
| 329506 | Ctdspl2 | 0.001613662 | 0.003200668 | 0.004569158 | 0.005026838 | 0.004016641 | 0 | 0 |
| 243867 | Fbxo46 | −0.00093222 | −3.39E−05 | −0.00148527 | −0.00309595 | −0.0015865 | 0 | 0 |
| 22139 | Ttr | −0.0025796 | −0.00032513 | 0.002230788 | 0.001874271 | −0.00047346 | 0 | 0 |
| 13726 | Emd | −0.00091951 | −0.00145801 | −0.00023754 | −0.00138821 | −0.00070404 | 0 | 0 |
| 74376 | Myo18b | −0.00192107 | 0 | −0.00470096 | −0.00493835 | −0.00285755 | 0 | 0 |
| 19385 | Ranbp1 | 0.00189027 | 0.000246877 | 0.002361602 | 0.00328815 | 0.002713038 | 0 | 0 |
| 231931 | Gimap6 | −0.00073471 | −0.0024555 | −0.00499705 | −0.00681021 | −0.00132903 | 0 | 0 |
| 170790 | Mlc1 | 0 | 0 | 0 | 0 | 0.00142157 | 0 | 0 |
| 12155 | BmpZ | 0.000154903 | 0.002745267 | 0.004503978 | 0.004325707 | 0.00290764 | 0 | 0 |
| 207728 | Pde2a | 0.000865816 | 0.001438157 | −0.00245099 | −0.00359223 | −0.00367234 | 0 | 0 |
| 15560 | Htr2c | 0.000411949 | −0.00141041 | −0.00148021 | −0.0016675 | −0.0004445 | 0 | 0 |
| 215615 | Rnpep | −0.0020839 | 0.001126405 | −0.00486059 | −0.00485572 | −0.00315103 | 0 | 0 |
| 98404 | Al597479 | 0.000876764 | −0.00046168 | −0.00041412 | −0.00058735 | −0.00040555 | 0 | 0 |
| 117589 | Asb7 | 0.001422528 | 0.000743653 | 0.003848683 | 0.003822881 | 0.003199058 | 0 | 0 |
| 214137 | Arhgap29 | 3.04E−05 | −0.00029906 | −0.00228656 | −0.00234617 | −0.00029539 | 0 | 0 |
| 230824 | Grhl3 | 0.000572911 | 0.002652616 | 0 | 0.002497957 | 0.001113322 | 0 | 0 |
| 672511 | Rnf213 | 0.002150095 | −0.00089847 | 0.000829011 | 0.004114913 | 0.002650167 | 0 | 0 |
| 19735 | Rgs2 | −0.00244364 | −0.00021197 | −0.00453841 | −0.00435843 | −0.00296159 | 0 | 0 |
| 14183 | Fgfr2 | 0.001322862 | 0.000831821 | −0.00012216 | 0.001478861 | 0.00174328 | 0 | 0 |
| 241226 | Itgs8 | −0.00195431 | −0.00164053 | −0.00232745 | −0.00259951 | −0.00569114 | 0 | 0 |
| 11363 | Acadl | 0.001827185 | 9.12E−06 | 0.001687431 | 0.002123296 | 0.002282657 | 0 | 0 |
| 105439 | Slain1 | 0.000775058 | 0 | 0.004339824 | 0.003281828 | 0.000869415 | 0 | 0 |
| 23880 | Fyb | 0.002719883 | −0.00160128 | 0.005240606 | 0.003940012 | 0.002776313 | 0 | 0 |
| 14561 | Gdf11 | −0.00132951 | 0.000208112 | 0.000173624 | −0.00100048 | −0.00085409 | 0 | 0.054102311 |
| 12842 | Col1a1 | −0.00721138 | 0.001915967 | −0.00135855 | −0.00681176 | −0.00518482 | 0 | −0.03159354 |
| 140703 | Emid1 | −0.00298459 | −0.00393407 | −0.00149045 | −0.0049929 | −0.00117769 | 0 | 0 |
| 227753 | Gsn | −0.00184645 | −0.00040474 | −0.00464628 | −0.00257793 | −0.0028754 | 0 | 0 |
| 17345 | Mki67 | −0.00132475 | −0.00238373 | −0.00514673 | −0.00500197 | −0.00498233 | 0 | 0.024678529 |
| 268977 | Ltbp1 | −0.00052389 | 0.000183629 | −0.00295646 | −0.00633135 | −0.00309465 | 0 | 0 |
| 74513 | Neto2 | −0.00128903 | 0 | −0.00162418 | −0.00230842 | −0.0050411 | 0 | 0 |
| 235627 | Nbeal2 | 0.001168979 | −0.00156541 | 0.002306314 | 0.002477003 | 0.000944949 | 0 | 0 |
| 17132 | Maf | 3.50E−05 | 0.000815949 | −0.00574545 | −0.00412377 | −0.00452069 | 0 | 0 |
| 67917 | Zcchc3 | −0.00103422 | 0.001226884 | −0.00570009 | −0.0061603 | −0.00297229 | 0 | −0.00024929 |
| 252972 | Tpcn1 | −0.00123618 | 0.000815056 | −0.0088145 | −0.00907784 | −0.00241265 | 0 | 0 |
| 17476 | Mpeg1 | 0.002598075 | −0.00173218 | 0.006381571 | 0.005045844 | 0.00499428 | 0 | 0 |
| 170742 | Sertad3 | 0.000783384 | 0.001179316 | 0.009522756 | 0.012723448 | 0.0067188 | 0 | 0 |
| 68701 | Ppp1r27 | 0 | 0 | 0 | 0 | 0.009897253 | 0 | 0 |
| 16519 | Kcnj3 | 0.001305399 | 0 | 0.007194017 | 0.004029593 | 0.003172105 | 0 | 0 |
| 16668 | Krt18 | 0.000399866 | 0.000312252 | 0.003132162 | 0.003044375 | 0.00016041 | 0 | 0 |
| 18762 | Prkcz | 0.003325481 | 0.003717142 | 0.00707634 | 0.007716986 | 0.00470695 | 0 | 0 |
| 60322 | Chst7 | −0.00027895 | −0.00060668 | 0.007243805 | 0.005483229 | 0.008733873 | 0 | 0 |
| 70779 | Prdm5 | −0.00177474 | 0 | −0.0051081 | −0.00747763 | −0.00469695 | 0 | 0 |
| 68099 | Cibar1 | −0.00163954 | −0.00178256 | −0.00388989 | −0.00673211 | −0.00329468 | 0 | 0 |
| 14049 | Eya2 | 0.001675027 | 0 | 0.003656726 | 0.005122814 | 0.004107038 | 0 | 0 |
| 16497 | Kcnab1 | 0.001111991 | 0.000224499 | 0.003504923 | 0.002284399 | 0.003536793 | 0 | 0.087342986 |
| 56224 | Tspan5 | 0.000524254 | −0.00011419 | −0.00287497 | −0.00561097 | −0.00424661 | 0 | 0 |
| 269016 | Sh3rf2 | 0.000487311 | −0.00096286 | 0.001078591 | 0.0001627 | −2.54E−05 | 0 | 0 |
| 11722 | Amy1 | 0.003207793 | 0.001397189 | 0.010927019 | 0.012672774 | 0.007915573 | 0 | 0 |
| 14588 | Gfra4 | −0.00280192 | −0.00105594 | −0.00213524 | −0.00259324 | −0.00250474 | 0 | 0 |
| 212937 | Tifab | −0.00221082 | −0.00125 | −0.00917238 | −0.00890447 | −0.0066399 | 0 | 0.024460823 |
| 239790 | Ostn | 0 | 0 | 0 | 0 | 0.004848232 | 0 | 0 |
| 19713 | Ret | −0.00148991 | −0.00042854 | −0.00349699 | 0.001036405 | −0.00234215 | 0 | 0 |
| 67419 | Armh4 | 0.00162198 | 0 | 0.003893044 | 0.005784516 | 0.003985015 | 0 | 0 |
| 11815 | Apod | 0.004909992 | 0 | 0.007655582 | 0.00698767 | 0.009671613 | 0 | 0 |
| 329872 | Frem1 | −0.00073546 | −0.00119233 | 0.00106387 | 0.001512971 | 0.000289825 | 0 | −0.02203615 |
| 208151 | Tmem132b | 0.000341553 | 0 | 0 | 0.003902325 | 0.002084789 | 0 | 0 |
| 22436 | Xdh | 0.003799714 | 0.000204983 | 0.003411969 | 0.002111416 | 0.002948139 | 0 | 0 |
| 319765 | Igf2bp2 | −0.00436618 | 0.001412059 | −0.00541868 | −0.00845622 | −0.005911 | 0 | 0 |
| 20196 | S100a13 | 0.00052496 | 0.001406818 | −0.00250546 | −0.00188771 | 0.000200192 | 0 | 0 |
| 170833 | Hook2 | 0.00098647 | −0.00425496 | 0.00039382 | 0.000533701 | 0.000344796 | 0 | 0 |
| 381148 | Prob1 | −0.00147679 | −0.00142112 | −0.00340994 | −0.00500161 | −0.00180516 | 0 | 0 |
| 76980 | Ube2ql1 | 0.001306678 | −0.00172394 | 0.002696782 | 0.003735793 | 0.001557603 | 0 | 0 |
| 396184 | Flrt1 | −0.00074152 | −0.00289989 | −0.00345421 | −0.00409934 | −0.00176 | 0 | 0.140333973 |
| 74596 | Cds1 | 0.002371296 | −0.00072518 | 0.005691121 | 0.006220588 | 0.003641342 | 0 | 0 |
| 105722 | Ano6 | 0.000417038 | 0.000461135 | 0.001068402 | −0.00037944 | 0.001590925 | 0 | 0 |
| 320997 | Cyp4f39 | 0.001297432 | 0.000958344 | 0.001347279 | 5.05E−05 | 0.00011427 | 0 | 0 |
| 13616 | Edn3 | −0.00233291 | −0.00037726 | 0.000387692 | −0.00014624 | −0.00272396 | 0 | 0 |
| 66714 | 4921524J17Rik | −0.00146323 | 0.001068841 | −0.00352984 | −0.00348869 | −0.00162576 | 0 | 0 |
| 110862 | Kcnq3 | 0.001827452 | −0.0004016 | 0.004907185 | 0.006443725 | 0.005132672 | 0 | 0 |
| 53311 | Mybph | 0.001044977 | 0 | 0.005089343 | 0.00372091 | 0.002945018 | 0 | 0 |
| 69065 | Chec1 | −0.00053283 | 0.001219545 | 0.004233411 | 0.00260704 | 0.002070028 | 0 | −0.19398905 |
| 234577 | Cpne2 | 0.003535026 | 0.006090621 | 0.006101999 | 0.00704417 | 0.005162748 | 0 | 0 |
| 629147 | Ctxn3 | 0.002440162 | 0 | 0 | 0.004058374 | 0.003602513 | 0 | 0 |
| 209448 | Hoxc10 | −0.00233631 | −0.00113473 | −5.80E−05 | 5.38E−05 | −0.00218877 | 0 | 0 |
| 12643 | Chad | −0.0032369 | 0.001248158 | −0.00233755 | −0.00152264 | −0.004006 | 0 | 0 |
| 67141 | Fbxo5 | 0.000235605 | 0.000389015 | −0.00020119 | −0.00110431 | −0.00035398 | 0 | 0 |
| 230775 | Adgrb2 | 0.001782481 | 0.002490454 | 0.00140756 | 0.009307175 | 0.008158283 | 0 | 0 |
| 19288 | Ptx3 | −0.00134453 | 0 | −0.00725184 | −0.01082669 | −0.0061388 | 0 | 0 |
| 17392 | Mmp3 | 0.002876865 | −0.0037989 | 0.004794796 | 0.004763901 | 0.00321651 | 0 | 0 |
| 19038 | Pplc | −0.00248177 | −0.00038294 | −0.00035771 | −0.00419001 | −0.00300396 | 0 | 0 |
| 223332 | Ranbp3l | −0.0012053 | 0.000599002 | −0.0009917 | −0.00330796 | −0.00218548 | 0 | 0.096648004 |
| 70021 | Nt5dc2 | −0.00290101 | 0.003229372 | −0.00745721 | −0.00500925 | −0.00230296 | 0 | 0 |
| 320633 | Zbtb26 | −0.00088304 | 0.00256765 | −0.00333469 | −0.00414627 | −0.00373381 | 0 | 0 |
| 11435 | Chrna1 | 0.001727524 | −4.62E−05 | −0.00036843 | 0.001222789 | 0.002403573 | 0 | 0 |
| 107765 | Ankrd1 | 0.002155932 | 0.002004983 | 0.000892739 | 0.003560702 | 0.004351262 | 0 | −0.02464597 |
| 170835 | Inpp5j | −0.00241116 | −0.00032176 | −0.00291085 | −0.0033056 | −0.00221701 | 0 | 0 |
| 74116 | Pi16 | −0.00171371 | −0.00182316 | −0.00398633 | −0.00410668 | −0.00076755 | 0 | 0 |
| 208501 | Ndufaf8 | −0.00141375 | −0.00060419 | −0.00753119 | −0.00792388 | −0.00369183 | 0 | 0 |
| 244853 | Nxpe4 | −0.00122419 | −0.00367479 | −0.00693835 | −0.00683989 | −0.00505277 | 0 | 0 |
| 17528 | Mp2 | −0.00167137 | −0.00039137 | 0.000136539 | −0.00450871 | −0.00281945 | 0 | 0 |
| 27260 | Plek2 | 0.000257872 | 0.001812339 | 0.004557766 | 0.004810151 | 0.00161974 | 0 | 0 |
| 20271 | Scn5a | 0.001710122 | 0 | 0.000961437 | −0.00115469 | 0.001960469 | 0 | 0 |
| 13717 | Eln | −0.00321537 | −0.00056255 | 0.003884276 | 0.001784298 | 0.000822765 | 0 | 0 |
| 77963 | Hook1 | 0.001362712 | −0.00096215 | −8.96E−05 | 0.002303776 | 0.00210966 | 0 | 0 |
| 233752 | Insc | 1.86E−06 | 0.003184171 | 0.003468159 | −0.00024865 | 0.000770089 | 0 | 0 |
| 245537 | Nlgn3 | −0.000625 | −2.93E−05 | −0.00196807 | −0.00169007 | −0.00157198 | 0 | 0 |
| 170720 | Card14 | −0.00015474 | 0.000904172 | −0.00081802 | 0.001067986 | −0.00054949 | 0 | 0 |
| 277743 | Fam131c | −0.00177627 | 0.001321258 | −0.0003895 | −0.00205371 | −0.00258028 | 0 | 0 |
| 83671 | Sytl2 | −0.00054396 | 0.001169963 | −0.00261546 | −0.00180186 | −0.00142614 | 0 | 0 |
| 72634 | Tarkh | 0.00233389 | −0.00012496 | 0.003138615 | 0.005677684 | 0.004525111 | 0 | −0.10039124 |
| 219228 | Pcdh17 | −0.00305824 | 0.001558365 | 0.000158514 | −0.00310361 | −0.00284343 | 0 | 0.055000342 |
| 14104 | Fasn | 0.000883739 | −0.00427543 | −0.00169344 | −0.00060828 | 0.002350361 | 0 | 0.154952204 |
| 382051 | Pdp2 | −0.00092999 | −0.00040602 | −0.00268737 | −0.00262668 | −0.00131613 | 0 | 0 |
| 73707 | Gucy2g | 0.000543322 | 0.00010666 | 0.001156321 | 0.001055355 | 0 | 0 | 0 |
| 17906 | Myl2 | 0.000138994 | 0 | 0.001585405 | 0.001830789 | 0.000859596 | 0 | 0 |
| 230163 | Aldob | 0.001134479 | 0.001360897 | −8.49E−06 | 6.77E−05 | −0.00023805 | 0 | 0 |
| 20512 | Slc1a3 | −0.00124976 | −0.0029777 | −0.00261982 | −0.00473505 | −0.00607712 | 0 | 0 |
| 20259 | Scin | 0.003232115 | −0.00217603 | −0.00137237 | 0.002054673 | 0.00082178 | 0 | 0 |
| 66425 | Pcp4l1 | −0.00097557 | −0.0001328 | 0.002824795 | 0.000370043 | −0.0021538 | 0 | 0 |
| 66402 | Sln | 0.000108915 | −0.00163193 | −0.00216613 | −0.00389734 | 0 | 0 | 0 |
| 18111 | Nnat | 0.000633581 | −0.00077234 | 0.004990829 | 0.00513214 | 0.003406098 | 0 | 0 |
| 72309 | Tmem158 | 0.002266578 | 0.00046299 | 0.0021348 | 0.003149525 | 0.000617359 | 0 | 0 |
| 13009 | Csrp3 | 0.000469051 | 0.001717681 | −0.00073673 | 3.33E−05 | −9.03E−05 | 0 | −0.26155009 |
| 17879 | Myh1 | 0.002016289 | 0.000347686 | 0.006680626 | 0.006867577 | 0.002666525 | 0 | 0 |
| 75219 | Dusp18 | −0.00215388 | 0 | −0.00190817 | −0.00311422 | −0.00301507 | 0 | 0 |
| 16790 | Anpep | 0.000116854 | −0.00196051 | −0.00064237 | −0.00171254 | 0.001479759 | 0 | 0.029392668 |
| 101772 | Ano1 | 0.000570527 | 0.000935884 | 0.001367727 | 0.001483969 | 0.002398169 | 0 | 0 |
| 20379 | Sfrp4 | −0.00069783 | 0.000574442 | 0.000644827 | 0.000906669 | 0.001607049 | 0 | 0 |
| 27273 | Pdk4 | 0.000882027 | 0.00434437 | 0.002859501 | 0.006159836 | 0.002247869 | 0 | −0.17705255 |
| 13723 | Emb | −0.00148577 | 6.14E−05 | −0.00521748 | −0.00605186 | −0.00394806 | 0 | 0 |
| 20464 | Sim1 | 0.000173624 | 0.000668016 | −0.00190173 | 0.00045994 | 0.001631709 | 0 | 0 |
| 277898 | Slc15a5 | −0.00057787 | 0.001188256 | 0.004842652 | 0.002849973 | 0.002255422 | 0 | 0.021728719 |
| 26878 | B3galt2 | 0.000172393 | 0 | 0.000413872 | −0.00072728 | 0.001031384 | 0 | 0 |
| 252864 | Dusp15 | −0.00080832 | 0 | −0.00527632 | −0.00283215 | −0.00128788 | 0 | 0 |
| 68797 | Pdgfrl | −0.00394436 | −0.00056241 | −0.00544561 | −0.00565613 | −0.00418627 | 0 | 0.107422395 |
| 72709 | C1qtnf6 | −0.00413177 | −0.00111368 | −0.00353987 | −0.00433785 | −0.00341634 | 0 | 0 |
| 12576 | Cdkn1b | −0.0018484 | 0.00116773 | −0.00081828 | −0.00022088 | −0.00034921 | 0 | 0 |
| 18053 | Ngfr | 0.003363856 | −0.00192949 | 0.00301688 | 0.004028603 | 0.003532428 | 0 | 0 |
| 17153 | Mal | −0.00124537 | −0.00389357 | −0.00311703 | −0.00398233 | −0.00380979 | 0 | 0 |
| 19124 | Procr | −0.00208299 | −0.00179937 | −0.00213377 | −0.00198287 | −0.00250387 | 0 | 0 |
| 109270 | Prr5 | −0.00066493 | −0.00204847 | −0.00139481 | −0.00034313 | −0.00118208 | 0 | 0 |
| 229599 | Ciart | 0.001884218 | −0.00073564 | 0.005876948 | 0.006916379 | 0.005612724 | 0 | −0.1374228 |
| 71839 | Osgin1 | 0.000399675 | −0.00129136 | −0.00580478 | −0.00573611 | −0.00098276 | 0 | 0 |
| 70134 | Misp3 | 0 | 0 | 0 | 0 | −0.0010065 | 0 | 0 |
| 20195 | S100a11 | −0.00099307 | −0.00380477 | −0.00508651 | −0.00468991 | −0.00364087 | 0 | 0 |
| 16846 | Lep | −0.00014248 | 0.001132766 | 0.002111403 | 0.001288254 | 0.001941109 | 0 | 0 |
| 11941 | Atp2b2 | −0.00084893 | −0.00268913 | −0.00215902 | −0.00131333 | −0.00306991 | 0 | 0 |
| 12575 | Cdkn1a | 0.006453696 | 0.003838183 | 0.007538629 | 0.013161546 | 0.012856729 | −0.08231495 | 0 |
| 17883 | Myh3 | 0.000979834 | 0 | 0 | 0.001012164 | 0.005411003 | 0 | 0 |
| 216188 | Aldh1l2 | −0.00128137 | −0.00177993 | −0.00170028 | −0.00438938 | −0.0049544 | 0 | 0 |
| 56437 | Rrad | 0.000949172 | 0 | 0.001125321 | 0.000446318 | 0.001384308 | 0 | 0 |
| 67044 | Higd2a | −0.00065387 | 0.000489005 | −0.00390376 | −0.00369265 | −0.00348186 | 0 | 0 |
| 140493 | Kcnn3 | 0.000119002 | 0.000210927 | −0.0017183 | −0.0001212 | 0.002344745 | 0 | 0 |
| 50877 | Neu3 | −0.00134475 | −0.00274496 | −0.00708877 | −0.008295 | −0.00510072 | 0 | 0.042820365 |
| 225164 | Mib1 | 0.001138489 | 0.001822868 | 0.004084247 | 0.003251592 | 0.002316473 | 0 | 0 |
| 319455 | Pld5 | 0.000298136 | 0 | 0 | 0.000584448 | 0.000684991 | 0 | 0 |
| 83922 | Cep41 | −0.00058972 | −0.00067226 | 0.000171148 | −0.00133931 | 0.000206122 | 0 | 0 |
| 319197 | Gpr4 | −0.00207124 | −0.00066259 | −0.00540784 | −0.00634212 | −0.00527295 | 0 | 0 |
| 81904 | Cacng7 | −0.00123642 | 0 | −0.0069023 | −0.00659997 | −0.00454026 | 0 | 0 |
| 109663 | Hoxc11 | 0 | 0 | 0 | 0 | 0 | 0 | 0 |
| 14555 | Gpd1 | 0.002793812 | 0.001731341 | 0.002528456 | 0.00283166 | 0.002138626 | 0 | 0 |
| 213469 | Lgi3 | −0.00090223 | −0.00080012 | −0.00161125 | −0.00101378 | −0.00080814 | 0 | 0 |
| 67096 | Mmachc | 0.00118401 | 0.001144944 | 0.000438398 | 0.00300482 | 0.001223198 | 0 | 0 |
| 101359 | Prrt4 | 0.000879471 | 0 | −0.00028198 | 0.002771474 | 0.001265006 | 0 | 0 |
| 20692 | Sparc | −0.00490344 | −0.00157506 | −0.00699521 | −0.00793429 | −0.00627687 | 0 | 0 |
| 110082 | Dnah5 | −0.00121638 | 0 | −0.00192104 | −0.00183339 | −0.00133279 | 0 | 0 |
| 20422 | Sem1 | −0.00091179 | −0.00021133 | −0.00059243 | −0.00131986 | −0.00033749 | 0 | 0 |
| 329252 | Lgr6 | −0.00055631 | −0.00043426 | −0.00149748 | −0.00506158 | −0.00208519 | 0 | 0 |
| 16367 | Irs1 | −0.00058134 | −0.00129591 | −0.00118645 | −0.00284473 | −0.00203191 | 0 | 0 |
| 76376 | Slc24a2 | 0.001068779 | 0 | 0.002171258 | 0.001581214 | 0.001619936 | 0 | 0 |
| 66337 | Fam229b | −0.0002346 | −0.00020008 | −0.00080588 | −0.0042294 | −0.0028476 | 0 | 0 |
| 13011 | Cst7 | 0.005890417 | 0.002153627 | 0.016064983 | 0.01736007 | 0.00960661 | 0 | 0 |
| 104886 | Rab15 | −0.00078912 | 0.000441946 | −0.00150024 | −0.00203259 | −0.00021861 | 0 | 0 |
| 13107 | Cyp2f2 | 0.002390408 | −0.00124642 | 0.007983395 | 0.007923176 | 0.007896141 | 0 | 0 |
| 16005 | Igfals | −0.00014885 | 0.003775544 | 0.004451769 | 0.003410952 | 0.002839349 | 0 | −0.36090239 |
| 208164 | Fam180a | 0.001016993 | 0.001259438 | 0.002565922 | 0.003693213 | 0.004514393 | 0 | 0 |
| 74748 | Slamf8 | 0.00145817 | −0.00178382 | −3.81E−05 | 0.002974937 | 0.002880285 | 0 | 0 |
| 14311 | Cidec | −0.00211052 | 0.000408763 | −0.00405961 | −0.00632172 | −0.00520091 | −0.04235353 | −0.01652478 |
| 15545 | Kera | −0.00155231 | 0.001765995 | −0.00156509 | −0.00055378 | −0.00088228 | 0 | 0 |
| 16019 | Ighm | 0.00660031 | 0 | 0.023260774 | 0.022128539 | 0 | 0 | 0 |
| 14080 | Fabp1 | −0.00082727 | 0.000945324 | 0.00280603 | 0.001322825 | 0.001255206 | 0 | 0.127591204 |
| 20716 | Serpina3n | 0.001400824 | −0.00444353 | 0.006430856 | 0.005848922 | 0.004362536 | 0 | 0 |
| 16411 | Itgax | 0.003998443 | 0.000520046 | 0.005475684 | 0.005800536 | 0.005642184 | 0 | 0 |
| 320360 | Rlc3 | 0.004678811 | 0.002918594 | 0.005094431 | 0.007807999 | 0.006099871 | 0 | 0 |
| 14619 | Gjb2 | 0.000102175 | −0.00068834 | −0.00426816 | −0.00348891 | −0.0012141 | 0 | 0 |
| 76293 | Mfap4 | −0.00434144 | −0.00194266 | −0.00640368 | −0.00736247 | −0.00817509 | 0 | 0 |
| 12843 | Col1a2 | −0.00524179 | −0.00252099 | −0.0084158 | −0.00750233 | −0.00608708 | 0 | 0 |
| 12869 | Cox8b | 0.001192623 | 0 | 0.002383276 | 0.003507988 | 0.003014122 | 0 | 0 |
| 16956 | Lpl | 0.000275956 | −0.00137076 | 0.004197341 | 0.003646261 | 0.000751777 | 0 | 0 |
| 23833 | Cd52 | 0.002265387 | −0.00108557 | 0.002801118 | −1.94E−05 | −0.00070005 | 0 | 0 |
| 12484 | Cd24a | −0.00204314 | −0.00464923 | −0.01021245 | −0.00771905 | −0.00524888 | 0 | 0 |
| 227485 | Cdh19 | 0.002830733 | 0.004243072 | 0.006018607 | 0.005741613 | 0.004637866 | 0 | 0 |
| 434178 | Zfp141 | 0.001682652 | 0.001950074 | 0.010422712 | 0.010625706 | 0 | 0 | 0 |
| 106014 | Tafa5 | 0.002795963 | 0.001474311 | 0.009772342 | 0.008804441 | 0.006769058 | 0 | 0 |
| 15937 | Ier3 | 0.002833721 | 0.000925914 | 0.005875611 | 0.003815341 | 0.00263368 | 0 | 0.021052303 |
| 16768 | Lag3 | 0.002636854 | −0.00046873 | 0.007714078 | 0.004431988 | 0.005428406 | 0 | −0.03371752 |
| 23943 | Esyt1 | 0.001525358 | 0.001051697 | 0.004869063 | 0.003697574 | 0.00282528 | 0 | −0.00333476 |
| 57262 | Retnla | −0.00265882 | 0 | −0.00566146 | −0.00470523 | 0 | 0 | 0 |
| 16601 | Klf9 | −0.001314 | −0.00159662 | −0.00648894 | −0.00574598 | −0.00567051 | 0 | 0 |
| 17085 | Ly9 | 0.002028409 | 0.000627382 | 0.001949033 | −0.00024709 | 0.001447878 | 0 | 0 |
| 12267 | C3ar1 | 0.00204008 | 0.002722735 | 0.003523365 | 0.003543239 | 0.003219519 | 0 | 0 |
| 14025 | Evl | −0.00351849 | −0.00330883 | −0.01009992 | −0.00851552 | −0.00563638 | 0 | 0 |
| 228094 | Cerkl | 0.002118591 | 0.003201743 | 0.005760487 | 0.002586186 | 0.004594514 | 0 | 0 |
| 22153 | Tubb4a | −0.00163099 | −0.00086343 | −0.00549351 | −0.00476535 | −0.00227573 | 0 | 0 |
| 20501 | Slc16a1 | 0.001655538 | 0.00087708 | 0.003030504 | 0.005604654 | 0.005422112 | 0 | 0 |
| 110557 | H2-Q6 | 0.002428432 | 0.000777606 | 0.009457556 | 0.007054385 | 0 | 0 | 0 |
| 17951 | Naip5 | 0.000784221 | 0.001212071 | 0.00243801 | 0.003509451 | 0.003541555 | 0 | 0 |
| 20666 | Sox11 | 0 | 0 | 0 | 0 | −0.00444955 | 0 | 0 |
| 15591 | Kl | −0.00081503 | 0.001374875 | −0.0044189 | −0.00533709 | −0.00672927 | 0 | 0 |
| 12642 | Ch25h | 0.002166432 | −0.00036068 | 0.003434342 | 0.0026497 | −0.00180424 | 0 | 0 |
| 214305 | Hhipl1 | 0.002182281 | 0.00112013 | 0.004175779 | 0.002547304 | 0.002835528 | 0 | 0 |
| 66548 | Adamtsl5 | 0.001975765 | 0.004583847 | 0.005681455 | 0.005283333 | 0.00506516 | 0 | 0 |
| 23972 | Papss2 | −0.00207446 | −0.0032222 | 0.00520278 | −0.00584955 | −0.00339395 | 0 | 0 |
| 54353 | Skap2 | 0.003395115 | 0.001196585 | 0.00712939 | 0.00683723 | 0.006628238 | 0 | 0 |
| 66102 | Cxcl16 | 0.003467717 | −0.00097705 | 0.006096342 | 0.007749944 | 0.0043575 | 0 | 0 |
| 59539 | Trnp1 | 0.002531976 | 0.00126597 | 0.008719707 | 0.009005579 | 0.007576843 | 0 | 0 |
| 74096 | Hvcn1 | 0.002395733 | 0.002736166 | 0.006504169 | 0.004982186 | 0.006167453 | 0 | 0 |
| 241230 | St8sia6 | 0.002906431 | 7.78E−05 | 0.00758425 | 0.007350522 | 0.005000443 | 0 | 0 |
| 64453 | Zfp280b | 0.001520904 | 0.001358833 | 0.006995179 | 0.008165554 | 0.00579352 | 0 | 0 |
| 18295 | Ogn | −0.00137535 | −0.00073554 | −0.0042494 | −0.00470037 | −0.00269549 | 0 | 0 |
| 53320 | Folh1 | 0.003108683 | −0.0019488 | 0.006280024 | 0.00640875 | 0.002992375 | 0 | 0 |
| 231991 | Creb5 | −0.00137729 | −0.00142367 | −0.00526645 | −0.0065333 | −0.00390767 | 0 | 0 |
| 14629 | Gclc | −0.00126904 | −0.00380815 | −0.00613118 | −0.00608512 | −0.00440516 | 0 | 0 |
| 407788 | BC051142 | 0.002045014 | −0.00068222 | 0.004887069 | 0.002760786 | 0.002736368 | 0 | 0 |
| 16997 | Ltbp2 | 0.002529507 | 0.000502863 | 0.002469221 | 0.003400932 | 0.004083279 | 0 | 0 |
| 237761 | Sowaha | −0.00111621 | 0 | −0.00558245 | −0.0052109 | −0.00416654 | 0 | 0 |
| 21753 | Tes | −0.00184952 | −0.00198923 | −0.00446121 | −0.0038866 | −0.00394744 | 0 | 0 |
| 140488 | Igf2bp3 | −0.00449538 | −0.00186812 | −0.00556921 | −0.00868941 | −0.00720602 | 0 | 0 |
| 17880 | Myh11 | −0.0018822 | −0.00628497 | −0.0055404 | −0.006442 | −0.00412537 | 0 | 0 |
| 12268 | C4b | 0.002758636 | −0.00267386 | 0.002373607 | 0.000240715 | 0.001704702 | 0 | 0 |
| 12478 | Cd19 | 0.00173095 | 0.003614509 | 0.003962727 | 0.005072307 | 0.004600078 | 0 | 0 |
| 12797 | Cnn1 | −0.00208752 | 0.001273355 | −0.00121582 | −0.00198521 | −0.00385923 | 0 | 0 |
| 52428 | Rhpn2 | 0.002095728 | 0.002207621 | 0.006113417 | 0.004882455 | 0.000170802 | 0 | 0 |
| 75739 | Mpp7 | −0.00115083 | −0.00036104 | −0.00801214 | −0.00731671 | −0.00709264 | 0 | 0 |
| 76757 | Trdn | 0.00244722 | 0 | 0.003975234 | 0.002367849 | 0.002873149 | 0 | 0 |
| 16529 | Kcnk5 | −0.00030206 | −0.00064707 | −0.00216803 | −0.00057735 | −0.00093339 | 0 | 0 |
| 11816 | Apoe | 0.003298621 | 0.000472926 | 0.006448352 | 0.00411273 | 0.005879206 | 0 | 0 |
| 109032 | Sp110 | 0.002371163 | 0.000759153 | 0.006072433 | 0.004531235 | 0.004518155 | 0 | 0 |
| 20292 | Ccl11 | 0.000643452 | −0.00096558 | 0.001945321 | −0.0009473 | 0.00027092 | 0 | 0 |
| 20897 | Stra6 | 0.002870379 | 5.84E−05 | 0.005545456 | 0.002700464 | 0.004396534 | 0 | 0 |
| 72097 | Cracdl | 0.001799723 | 0 | 0.003873739 | 0.002852233 | 0.003906762 | 0 | 0 |
| 216864 | Mgl2 | −0.00225077 | 0 | −0.00850441 | −0.0076857 | −0.00483155 | 0 | 0 |
| 242691 | Gpatch3 | 0.001041322 | 0.001821931 | 0.010176263 | 0.007717555 | 0.004289029 | 0 | 0 |
| 71829 | Ddi1 | 0 | 0 | 0 | 0 | 0.004997572 | 0 | 0 |
| 243653 | Clec1a | 0.002037635 | 0.001955744 | 0.007059297 | 0.00563138 | 0.001239197 | 0 | 0 |
| 56838 | Ccl28 | −0.00044851 | 0.000307673 | −1.99E−05 | −0.00025839 | 0.001049461 | 0 | 0 |
| 239318 | Plcxd3 | 0.002662956 | 0 | 0.007579045 | 0.007026596 | 0.005346985 | 0 | 0 |
| 233405 | Vps33b | −0.00103192 | −0.0018252 | −0.00421668 | −0.00433605 | −0.00438695 | 0 | 0 |
| 13004 | Ncan | −0.00156716 | −0.0004649 | −0.00302392 | −0.00357406 | −0.00066248 | 0 | 0 |
| 11537 | Cfd | −0.00129359 | 0.001874899 | −0.00379147 | −0.00214268 | −0.00099178 | 0 | −0.30834957 |
| 215436 | Slc35e3 | 0.001551695 | 0.003322751 | 0.002674274 | 0.004016754 | 0.004150022 | 0 | 0 |
| 12262 | C1qc | 0.003016731 | 0.00170831 | 0.006340999 | 0.006393711 | 0.004728272 | 0 | 0 |
| 24108 | Ubd | 0.003148796 | −0.00287608 | −0.00259311 | −0.00061791 | 0.001626833 | 0 | 0.064525081 |
| 20564 | Slit3 | 0.002918924 | 0.001477855 | 0.004547704 | 0.005880586 | 0.005076324 | 0 | 0 |
| 20307 | Ccl8 | 0.002745179 | 0.001298579 | 0.004339161 | 0.004318263 | 0 | 0 | −0.05857745 |
| 93885 | Pcdhb14 | 0.001798672 | 0.000649626 | 0.007919265 | 0.010566803 | 0.00577229 | 0 | 0 |
| 27053 | Asns | 0.001979813 | −0.00358199 | 0.001533796 | 0.001145634 | 0.003514248 | 0 | 0 |
| 69480 | Ttc9 | 0.001879336 | 0 | 0.005047623 | 0.003101756 | 0.00249887 | 0 | 0 |
| 17329 | Cxcl9 | −0.00252387 | 0.001769762 | −0.00705834 | −0.00740255 | −0.00457448 | 0 | 0.132100469 |
| 71519 | Cyp2u1 | 0.001300891 | −0.00064045 | 0.005068582 | 0.005524239 | 0.002503365 | 0 | 0.019318736 |
| 12826 | Col4a1 | −0.00436123 | −0.00171958 | −0.00496392 | −0.00528707 | −0.00648754 | 0 | 0 |
| 53356 | Eif3g | 0.001619472 | −0.00049099 | 0.00715807 | 0.00716369 | 0.002036535 | 0 | 0 |
| 56429 | Dpt | −0.00306316 | −0.00525256 | −0.00558165 | −0.00434535 | −0.0040645 | 0 | 0 |
| 68969 | Eif1b | 0.001903871 | −0.0006393 | 0.005058532 | 0.005287203 | 0.002148789 | 0 | 0 |
| 74202 | Fblim1 | 0.001043835 | 0.004090775 | 0.006096123 | 0.003920373 | 0.002536368 | 0 | 0 |
| 213417 | Klhdc8a | 0.001531366 | −0.00129985 | 0.005466208 | 0.00495598 | 0.005079028 | 0 | 0.0349557 |
| 237711 | Eml6 | 0.001632543 | 0.003508453 | 0.007172117 | 0.007470668 | 0.005328941 | 0 | 0 |
| 12819 | Col15a1 | −0.00235485 | 0.001850579 | −0.00209084 | −0.00213053 | −0.00298459 | 0 | −0.20235998 |
| 76654 | Upp2 | −0.00164223 | −0.00133047 | −0.00258409 | −0.00331729 | −0.00284562 | 0 | 0 |
| 58916 | Myot | 0.002320232 | 0 | 0.006984297 | 0.007491432 | 0.008207117 | 0 | 0 |
| 18008 | Nes | 0.002448198 | 0.001840178 | 0.008179555 | 0.006854401 | 0.006302454 | 0 | 0 |
| 217705 | Fam161b | 0.001612786 | 0.004347324 | 0.006021297 | 0.006494865 | 0.001732263 | 0 | 0 |
| 101314 | Brk1 | 0.000857016 | 0.001287079 | 0.005114956 | 0.006407841 | 0.002884047 | 0 | 0 |
| 18712 | Pim1 | 0.001466399 | 0.001498763 | 0.001273438 | −0.00020019 | 0.0001013 | 0 | 0 |
| 71984 | Sars2 | −0.00202654 | −0.00146936 | −0.00616459 | −0.00623151 | −0.00453814 | 0 | 0 |
| 23796 | Aplnr | −0.00343951 | 0.000509088 | −0.00521856 | −0.00517118 | −0.0006596 | 0 | 0 |
| 104601 | Mycbpap | −0.00164411 | −0.00046279 | −0.00259265 | −0.00137659 | −0.00263311 | 0 | 0 |
| 23954 | Nek3 | −0.00177821 | −0.00264703 | −0.00585358 | −0.00575179 | −0.0041035 | 0 | 0 |
| 72114 | Zbed3 | −0.00163629 | 0.003695724 | −0.00627935 | −0.00294144 | −0.00233993 | 0 | −0.07880316 |
| 58243 | Nap1l5 | 0.001956136 | 0.003298459 | 0.005215942 | 0.004962569 | 0.005406133 | 0 | 0 |
| 239554 | Foxred2 | 0.001537323 | 0.00173172 | 0.004139318 | 0.003835371 | 0.004253498 | 0 | 0 |
| 16142 | Iglv1 | 0 | 0 | 0 | 0 | 0 | 0 | 0 |
| 57339 | Jph1 | 0.002104509 | 0.000245476 | 0.005129833 | 0.005292739 | 0.005719155 | 0 | 0 |
| 17110 | Lyz1 | 0.00090034 | 0.002761424 | 0.000890746 | 0.000188465 | 0 | 0 | 0 |
| 75311 | 4930550C14Rik | −0.00203405 | 0.000129052 | −0.00530523 | −0.0063129 | −0.0037655 | 0 | 0 |
| 24099 | Tnfsf13b | 0.002332491 | 0.000486725 | 0.004390303 | 0.003750424 | 0.002828026 | 0 | 0 |
| 71781 | Slc16a14 | −0.00127532 | 0 | 0 | −0.00533384 | −0.0043303 | 0 | 0 |
| 68404 | Nrn1 | 0.001628172 | 6.41E−05 | 0.002296458 | 0.000138266 | 0.002051909 | 0 | 0 |
| 404634 | Macroh2a2 | −0.00209408 | −0.00233415 | −0.00524961 | −0.00507437 | −0.00325996 | 0 | 0 |
| 18985 | Pouzaf1 | 0.002797661 | 0.000710713 | 0.004690305 | 0.005379966 | 0.004426767 | 0 | −0.10589075 |
| 20183 | Rxrg | 0.00184368 | 0.003046503 | 0.005667635 | 0.003692197 | 0.001664933 | 0 | 0 |
| 74199 | Vlt | 0.001286097 | 0 | 0.002636879 | 0.002342696 | 0.001328909 | 0 | 0 |
| 12182 | Bst1 | −0.00115147 | 0.002048475 | −0.00090483 | −0.00034561 | −0.00183155 | 0 | 0 |
| 320502 | Lmod3 | −0.00071656 | 0 | −0.00450723 | −0.00349238 | −0.00239904 | 0 | 0 |
| 232987 | B9d2 | −0.00054614 | −0.00211292 | −0.00475386 | −0.0036213 | −0.00269568 | 0 | 0 |
| 15211 | Hexa | 0.001139189 | 0.001741621 | 0.002945305 | 0.004318629 | 0.003322922 | 0 | 0 |
| 21949 | Tnfsf8 | 0.001395747 | −0.00076386 | 0.006791766 | 0.004542687 | 0.003807056 | 0 | 0 |
| 238328 | Vash1 | −0.00290641 | −0.00218204 | −0.00462184 | −0.00430376 | −0.00193284 | 0 | 0 |
| 219189 | Vwa8 | 0.001582359 | 0.001159255 | 0.005720365 | 0.005531388 | 0.005452281 | 0 | 0.087680823 |
| 56248 | Ak3 | −0.00146534 | 0.000955601 | −0.00444729 | −0.00327587 | −0.00353151 | 0 | 0 |
| 224691 | Zfp472 | −0.00132412 | 0.001300741 | −0.00736534 | −0.00901356 | −0.00324597 | 0 | 0 |
| 12508 | Cd53 | 0.000363063 | 0.001551358 | 0.001254258 | 0.004791789 | 0.002028697 | 0 | 0 |
| 52276 | Cdca8 | −0.00242494 | −0.00087548 | −0.00381111 | −0.00176113 | −0.00324085 | 0 | 0 |
| 233064 | Wdr62 | −0.00188963 | −0.00217012 | −0.00557788 | −0.00591609 | −0.00378894 | 0 | 0 |
| 546611 | Klhl33 | −0.00114631 | 0 | −0.00482777 | −0.00370912 | 0 | 0 | 0 |
| 12982 | Csf2ra | −0.00144314 | −0.00411264 | −0.00310774 | −0.00179957 | −0.00273249 | 0 | 0 |
| 68867 | Rnf122 | −0.00285528 | −0.00117057 | −0.00455927 | −0.00360424 | −0.00268899 | 0 | 0 |
| 434341 | Nlrc5 | 0.003974339 | −0.00140528 | 0.005350424 | 0.005293207 | 0.003753278 | 0 | 0 |
| 58859 | Efemp2 | 0.002459785 | 0.002173859 | 0.007505225 | 0.007084928 | 0.004905158 | 0 | 0 |
| 15507 | Hspb1 | −0.00094194 | 0.001329648 | −0.00331782 | −0.00418939 | −0.00561692 | 0 | −0.04132811 |
| 98256 | Kmo | −0.00284647 | −0.00063983 | −0.00651144 | −0.00176011 | −0.00096641 | 0 | 0 |
| 114716 | Spred2 | −0.00156018 | −0.00288764 | −0.00673303 | −0.00621867 | −0.0041174 | 0 | 0 |
| 16009 | Igfbp3 | −0.00133407 | −0.00172808 | −0.00697108 | −0.00429521 | −0.00389557 | 0 | 0 |
| 19260 | Ptpn22 | 0.002591576 | −0.00135424 | 0.003260298 | 0.003084192 | 0.00266721 | 0 | 0.077328455 |
| 16185 | Il2rb | −0.00200834 | −0.00516019 | −0.00765098 | −0.00560133 | −0.0048159 | 0 | 0.07516761 |
| 67158 | Sft2d3 | 0.000768823 | 0.001161355 | 0.004846141 | 0.004304819 | 0.004056305 | 0 | 0 |
| 93882 | Pcdhb11 | 0.001120943 | 0.000565993 | 0.004800495 | 0.004935353 | 0 | 0 | 0 |
| 14605 | Tsc22d3 | −0.00213705 | 0.003298869 | −0.00329829 | −0.00274255 | −0.00298777 | 0 | 0 |
| 75472 | Cfap126 | −0.00059665 | −0.00272848 | −0.00414478 | −0.00437559 | −0.00192493 | 0 | 0 |
| 70536 | Qpct | 0.000661171 | −0.00036535 | 0.002268989 | 0.001021964 | 0.002620708 | 0 | 0.044281143 |
| 72572 | Spats2 | 0.001016385 | 0.000846427 | 0.005081176 | 0.003811384 | 0.001201302 | 0 | 0 |
| 19218 | Ptger3 | 0.002110059 | 0.001394359 | 0.007834206 | 0.009437762 | 0.003284113 | 0 | −0.04599522 |
| 100034361 | Mfap1b | 0.001097007 | 0.003219751 | 0.003942951 | 0.003415432 | 0.004700558 | 0 | 0 |
| 218820 | Zfp5O3 | 0.001650358 | 0.001183181 | 0.003543579 | 0.004394578 | 0.003503846 | 0 | 0.03591774 |
| 20474 | Slx4 | 0.001527536 | 0 | 0.006285282 | 0.006799935 | 0.00549759 | 0 | 0 |
| 15476 | Hs3st1 | −0.00212391 | −0.0003253 | −0.00625671 | −0.00640362 | −0.00559642 | 0 | 0 |
| 73739 | Cby1 | −0.00087322 | 0.000609401 | −0.00546757 | −0.00538489 | −0.00442593 | 0 | 0 |
| 69956 | Ptcd3 | 0.001630101 | −0.00136317 | 0.003904152 | 0.004917746 | 0.005572915 | 0 | 0 |
| 107528 | Magee1 | 0.000755505 | −0.00223184 | 0.003853167 | 0.003310975 | 0.004034808 | 0 | 0 |
| 29857 | Mapk12 | 0.002550451 | −1.22E−05 | 0.001857764 | 0.003276085 | 0.003685058 | 0 | 0 |
| 12902 | Cr2 | −0.00191629 | −0.0013219 | −0.00675149 | −0.00566398 | −0.00447261 | 0 | 0 |
| 268709 | Fam107a | 0.002441146 | 0 | 0.002288594 | 0.004265684 | 0.002073453 | 0 | 0 |
| 170574 | Sp7 | 0.001243794 | 0.000919683 | 0.007534164 | 0.007808942 | 0.004833626 | 0 | 0 |
| 23967 | Osr1 | −0.00205821 | 9.68E−05 | −0.00500287 | −0.00476606 | −0.00432406 | 0 | 0 |
| 16538 | Kcns1 | 0 | 0 | 0.004187539 | 0 | 0.003950377 | 0 | 0 |
| 21838 | Thy1 | 0.000849315 | 0.00035451 | 0.004108253 | 0.000616647 | 8.45E−05 | 0 | 0 |
| 14181 | Fgfbp1 | 0.001065284 | 0 | 0.004536491 | 0.004329553 | 0.005598784 | 0 | 0 |
| 213603 | Slc44a3 | −0.00326165 | −0.00101205 | −0.00441143 | −0.00222285 | −0.00329596 | 0 | 0.268308582 |
| 20618 | Sncg | 0.004958137 | −5.09E−05 | 0.006927994 | 0.006813459 | 0.008168052 | 0 | 0 |
| 59083 | Fetub | 0.002696423 | −0.00034067 | 0.005938825 | 0.003130994 | 0.001593931 | 0 | 0.076250075 |
| 74646 | Spsb1 | 0.00190718 | 0.003394669 | 0.005480689 | 0.007200529 | 0.001754063 | 0 | 0 |
| 56753 | Tacstd2 | 0.001163046 | 0 | 0.001302225 | 0.001190797 | 0.000559896 | 0 | 0 |
| 53313 | Atp2a3 | 0.002619532 | 0.002225889 | 0.004061559 | 0.003391917 | 0.004133401 | 0 | 0 |
| 67442 | Retsat | 0.001725028 | 0.002156773 | 0.005053772 | 0.005960028 | 0.005386257 | 0 | 0.053111605 |
| 16881 | Llg1 | 0.001291093 | −0.00025727 | 0.003413959 | 0.003806236 | 0.003230982 | 0 | 0 |
| 19009 | Pou6f1 | 0.002496491 | 0.003576743 | 0.00265048 | 0.004571518 | 0.003043877 | 0 | −0.07186251 |
| 18616 | Peg3 | −0.00123011 | −0.00181187 | −0.00602506 | −0.0045323 | −0.0028637 | 0 | −0.11015703 |
| 106931 | Ketd1 | 0.001302488 | 0.000940736 | 0.002784391 | 0.002736196 | 0.00148603 | 0 | 0 |
| 54156 | Egfl6 | −0.00061692 | −0.00129573 | −0.00155756 | 1.74E−05 | −0.00071739 | 0 | 0 |
| 13645 | Egf | −0.00193879 | −0.00154322 | −0.00579051 | −0.0058729 | −0.00643592 | 0 | 0 |
| 76108 | Rap2a | −0.00160206 | −0.00081094 | −0.00177217 | −0.00115092 | −0.00249166 | 0 | 0 |
| 66432 | Slc7a6os | 0.00082467 | 0.00266756 | −0.00606807 | −0.00530522 | −0.00374565 | 0 | 0 |
| 216616 | Efemp1 | 0.002724259 | 0.002050117 | 0.007704358 | 0.007526225 | 0.005059211 | 0 | 0 |
| 259097 | Olfr558 | −0.00213256 | 0 | −0.00124254 | −0.00031375 | −0.00051018 | 0 | 0 |
| 58994 | Smpd3 | −0.00275934 | −0.00461894 | −0.00205191 | −0.00503 | −0.0035915 | 0 | 0 |
| 18159 | Nppc | −0.000821 | 0.000520544 | −0.00478537 | −0.00280979 | −0.0031029 | 0 | 0 |
| 14201 | Fhl3 | −0.00171922 | −0.00092439 | −0.00389984 | −0.00516915 | −0.0030364 | 0 | 0 |
| 70099 | Smc4 | −0.00283641 | −0.00195121 | −0.00613214 | −0.00758205 | −0.00628493 | 0 | 0 |
| 13136 | Cd55 | −0.00217996 | −0.00218009 | −0.00274077 | −0.00159756 | −0.00265662 | 0 | 0 |
| 18817 | Plk1 | −0.00160675 | 0.000122117 | −0.00879405 | −0.00891249 | −0.00511101 | 0 | 0 |
| 102502 | Pls1 | 0.001572076 | −0.00395788 | 0.003689214 | 0.004083793 | 0.004153277 | 0 | 0 |
| 67893 | Tmem86a | −0.00121823 | 0.000849739 | −0.00440423 | −0.00581297 | −0.00387132 | 0 | −0.01439752 |
| 71956 | Rnf135 | 0.000594957 | −0.0004968 | 0.004238373 | 0.004554358 | 0.003988916 | 0 | 0 |
| 21881 | Tkt | 0.00111728 | −0.00033216 | 0.001986416 | 0.002829079 | 0.000817908 | 0 | 0 |
| 54451 | Cpsf3 | −0.00105823 | −0.00107641 | −0.00710311 | −0.00722812 | −0.00592989 | 0 | 0 |
| 15957 | Ifit1 | 0.002188273 | 0.00085658 | 0.006465004 | 0.006201027 | 0.005983039 | 0 | 0 |
| 72560 | Nealad2 | 0.000587117 | 0.00152936 | 0.007680199 | 0.00591875 | 0.004279549 | 0 | 0 |
| 328967 | Arhgef37 | 0.001299753 | −0.00131988 | 0.006794191 | 0.003891009 | 0.00214103 | 0 | −0.18449756 |
| 12702 | Socs3 | 0.001951461 | −7.21E−05 | 0.001078818 | 0.000315204 | 0.001683407 | 0 | 0.089968007 |
| 213393 | Depp1 | 0.002189525 | −0.00200767 | 0.005787295 | 0.005249067 | 0.003439775 | 0 | 0 |
| 242721 | Klhdc7a | 0.000919061 | 0.001067908 | 0.004497712 | 0.0073826 | 0.004011013 | 0 | 0 |
| 320825 | Samd5 | −0.00239677 | 0 | −0.00479628 | −0.00445642 | −0.00495062 | 0 | 0 |
| 72507 | Dzip1l | 0.00059149 | 0.001741742 | 0.001881693 | 0.001814202 | 8.37E−05 | 0 | 0 |
| 66859 | Slc16a9 | −0.00169197 | −0.00352128 | −0.00639794 | −0.00699183 | −0.00462474 | 0 | 0 |
| 16776 | Lama5 | 0.001632831 | −0.00171669 | 0.002370743 | 0.001590705 | 0.002709391 | 0 | 0 |
| 21922 | Clec3b | −0.00156118 | 0.000903675 | −0.00328478 | 8.34E−05 | −0.00383364 | 0 | 0 |
| 80901 | Cxcr6 | 0.001649045 | −0.00135627 | 0.001893509 | 0.000786912 | 0.001322308 | 0 | 0 |
| 11488 | Adam11 | −0.00148633 | −0.00024554 | −0.00327908 | −0.00509033 | −0.00543772 | 0 | 0.064087476 |
| 20514 | Slc1a5 | −0.00151227 | −0.00472769 | −0.00562366 | −0.00568207 | −0.00370888 | 0 | 0 |
| 384281 | Gatc | −0.00034592 | −0.00083391 | −0.00187322 | −0.00177958 | −0.00196751 | 0 | 0 |
| 229900 | Gbp7 | −0.00143494 | −0.00265409 | −0.00411127 | −0.0065933 | −0.00340542 | 0 | 0 |
| 29876 | Clic4 | 0.001803361 | −0.00078199 | 0.002614478 | 0.001478542 | 0.002848147 | 0 | 0 |
| 94214 | Spock2 | 0.002018245 | −0.00037105 | 0.005283005 | 0.007882148 | 0.005563017 | 0 | 0 |
| 192198 | Lrrc4 | −0.00121474 | 0 | −0.00641607 | −0.00672827 | −0.00278115 | 0 | 0 |
| 22715 | Zfp57 | −0.00155489 | 0.000205931 | −0.00586194 | −0.00393913 | −0.00298627 | 0 | 0 |
| 75744 | Svip | 0.001595524 | 0.00037037 | 0.00537992 | 0.004145258 | 0.004730577 | 0 | 0 |
| 68743 | Anln | 0.000799973 | 0.003164153 | 0.005507527 | 0.00413403 | −0.00027312 | 0 | 0 |
| 207798 | Gramd1c | 0.002134719 | 0.002006701 | 0.001776902 | 0.00395347 | 0.00344949 | 0 | 0 |
| 212733 | Bicdl2 | −0.00081518 | −0.00024436 | −0.00331836 | −0.0026685 | −0.00327905 | 0 | 0 |
| 245902 | Ccdc15 | 0.001575859 | −7.42E−05 | 0.005648411 | 0.0076305 | 0.004830871 | 0 | 0 |
| 12816 | Col12a1 | −0.00289006 | −0.00169207 | −0.00613223 | −0.00619774 | −0.00415894 | 0 | 0 |
| 66983 | Zfp830 | −0.00159645 | 0.000267576 | −0.00537924 | −0.00572288 | −0.00417235 | 0 | 0 |
| 110135 | Fgb | −0.00011544 | 0.0035948 | 0.000174831 | 0.000777362 | −0.00019161 | 0 | 0 |
| 58909 | Fam13a | −0.000422 | 0.002171062 | −0.00518313 | −0.00375058 | −0.00199471 | 0 | 0 |
| 242939 | Cpz | 7.72E−05 | −0.00107479 | 0.007289702 | 0.005400228 | 0.002291848 | 0 | 0 |
| 18231 | Nxph1 | 0.002235549 | 3.78E−06 | 0.002622831 | 0.002562507 | 0.001356793 | 0 | 0 |
| 14792 | Lpcat3 | −0.00251681 | −0.00242707 | −0.00501483 | −0.0049116 | −0.00340201 | 0 | 0 |
| 18979 | Pon1 | −0.00033382 | −0.00135984 | −0.00023479 | 0.000725341 | 0.000805356 | 0 | 0 |
| 207742 | Rnf43 | 0.001734446 | 0.003188553 | 0.006304002 | 0.006827901 | 0.00470301 | 0 | 0 |
| 170743 | Tlr7 | 0.002171354 | 0.000411361 | 0.005157037 | 0.005147568 | 0.002530145 | 0 | 0 |
| 12224 | Klfs | 0.001853527 | 0 | 0.007785077 | 0.006125444 | 0.005388124 | 0 | 0 |
| 227377 | Farp2 | 0.002061475 | 0.002154992 | 0.005943042 | 0.006601149 | 0.003561792 | 0 | 0 |
| 105785 | Kdelr3 | −0.00291369 | 0.002311356 | −0.00443905 | −0.00191061 | −0.00207862 | 0 | 0 |
| 270210 | Zfp651 | −0.00125634 | −0.00190175 | −0.00138117 | −0.00130044 | −0.00201995 | 0 | 0 |
| 27226 | Pla2g7 | 0.002132653 | −0.00123334 | −0.00372086 | −0.00070059 | 0.001506309 | 0 | −0.00423699 |
| 223665 | CO30006K11Rik | 0.001824419 | −0.00055419 | 0.004761296 | 0.005497246 | 0.005166504 | 0 | 0 |
| 12483 | Cd22 | 0.00286108 | 0.002303541 | 0.005201441 | 0.009587244 | 0.006378465 | 0 | 0 |
| 54667 | Atp8b2 | −0.00138764 | −0.00356989 | −0.00397472 | −0.00552973 | −0.00269352 | 0 | 0 |
| 230837 | Asap3 | −0.00234454 | 0.00021459 | −0.00374133 | −0.00283656 | −0.0021943 | 0 | 0 |
| 102436 | Lars2 | 0.001265826 | −0.00284 | 0.002588616 | 0.002487202 | 0.001237696 | 0 | 0 |
| 78892 | Crispld2 | −0.00138967 | −0.00163875 | −0.00592169 | −0.00554014 | −0.00480122 | 0 | 0 |
| 234564 | Cesif | −0.00254631 | 0.001866285 | 0.00100784 | 0.002248663 | 7.32E−05 | 0 | 0 |
| 56226 | Espn | 0.001464143 | 0.000433373 | 0.001736898 | 0.000629771 | −8.69E−05 | 0 | 0 |
| 68251 | Babam1 | 0.000266145 | 0.000242461 | 0.002348804 | 0.002718293 | 0.001846923 | 0 | 0 |
| 21386 | Tbx3 | 0.00151773 | 0.003741295 | 0.004582879 | 0.005344295 | 0.001613779 | 0 | 0.097852676 |
| 68667 | Trpm4 | 0.002337198 | 0.001841684 | 0.006909182 | 0.007565526 | 0.006199979 | 0 | 0 |
| 98267 | Stk17b | −0.00095051 | 0.001033509 | −0.0014783 | −0.004115 | −0.00236245 | 0 | −0.13608728 |
| 16182 | Il18r1 | 0.000921955 | 0.000102852 | 0.004908569 | 0.004076654 | 0.002250698 | 0 | 0 |
| 68705 | Gtf2f2 | 0.000306028 | −0.00251416 | −0.00052096 | −0.00071899 | −2.10E−07 | 0 | 0 |
| 330096 | Shisa3 | 0.002015731 | 0.001004075 | 0.005996974 | 0.00640421 | 0.003363448 | 0 | 0 |
| 54120 | Gipc2 | 0.00139226 | 0.000279784 | 0.002907274 | 0.003199018 | 0.001384682 | 0 | 0 |
| 13642 | Efnb2 | −0.00171779 | −0.00287578 | −0.0048058 | −0.00524566 | −0.00421005 | 0 | 0 |
| 20202 | S100a9 | 0.00330832 | 0.004712031 | 0.01041114 | 0.007892542 | 0.009004952 | 0 | 0 |
| 69528 | 1700030J22Rik | 0.000908463 | 0 | 0.003886686 | 0.00446963 | 0.004418177 | 0 | 0 |
| 12950 | Hapln1 | −0.00046193 | −0.00041469 | −0.00141978 | −0.00124012 | 0.00048129 | 0 | 0 |
| 243725 | Ppp1r9a | −0.0030344 | −0.00476328 | −0.00913452 | −0.00854325 | −0.00534553 | 0 | 0.166201817 |
| 52466 | Slc46a1 | −0.00291322 | −0.00190271 | −0.00469128 | −0.0049577 | −0.0032488 | 0 | 0 |
| 98170 | Tmem132a | −0.00277572 | −0.00552095 | 0.00568453 | −0.00557464 | −0.00618661 | 0 | 0 |
| 75146 | Mfsd13a | 0.002155651 | 0.001551024 | 0.005450127 | 0.00438758 | 0.003406516 | 0 | 0 |
| 21833 | Thra | 0.001115651 | 0.000798623 | 0.003804736 | 0.003433501 | 0.003020549 | 0 | 0 |
| 72400 | Pinx1 | 0.000681727 | 0.002103742 | 0.004208691 | 0.004362363 | 0.003234246 | 0 | 0 |
| 384061 | Fndc5 | −0.00298308 | −0.00105257 | −0.00373402 | −0.0042489 | −0.00298154 | 0 | 0 |
| 52700 | Txndc17 | −0.00071905 | −0.00224709 | −0.00301545 | −0.00283202 | −0.00195665 | 0 | 0 |
| 13482 | Dpp4 | −0.00238828 | −0.00047112 | −0.00203308 | −0.00303133 | −0.0019361 | 0 | 0 |
| 21892 | Tll1 | −0.0021758 | 0 | −0.00530035 | −0.00647226 | −0.00326044 | 0 | 0 |
| 66109 | Tspan13 | −0.0013687 | −0.00146105 | −0.00439555 | −0.00321289 | −0.00376334 | 0 | 0 |
| 17084 | Ly86 | 0.001308664 | 0.001973949 | 0.000897969 | 0.001485971 | 0.001789538 | 0 | 0.100235723 |
| 20280 | Scp2 | 0.001541211 | 0.00261171 | 0.001935786 | 0.002125617 | 0.002835075 | 0 | 0 |
| 331487 | Uprt | −0.00123651 | −0.00023541 | −0.00179475 | −0.0028964 | −0.00343849 | 0 | 0 |
| 218973 | Wdhd1 | 0.000472243 | −0.00067655 | 0.005245496 | 0.004208309 | 0.002561882 | 0 | 0 |
| 66694 | Uqcrfs1 | −0.00121642 | −0.00088255 | −0.00487948 | −0.00476263 | −0.0031326 | 0 | 0 |
| 64292 | Ptges | 0.00013066 | 0.000891942 | 0.001319774 | 0.001948776 | 0.001982281 | 0 | 0 |
| 17022 | Lum | 0.000516458 | 0.001983838 | 0.001847082 | 0.002669298 | 0.002748374 | 0 | −0.03392318 |
| 52906 | Ahi1 | 0.001888324 | 0.001879444 | 0.004607044 | 0.004636231 | 0.00293316 | 0 | 0 |
| 545370 | Hmcn1 | −0.00058335 | 0.001501956 | 0.003591396 | 0.002280951 | −0.00029445 | 0 | 0 |
| 26432 | Plod2 | −0.00245319 | −0.00332965 | −0.00575074 | −0.00385966 | −0.00256963 | 0 | 0 |
| 103710 | Slc35e4 | −0.00070018 | −0.00272624 | −0.00812006 | −0.00853747 | −0.00234089 | 0 | 0 |
| 110948 | Hlcs | 0.002334529 | 0.00235613 | 0.004664766 | 0.003426114 | 0.002710909 | 0 | 0 |
| 13200 | Ddost | 0.001074142 | 0.002448203 | 0.003994018 | 0.004372879 | 0.003043451 | 0 | 0 |
| 21687 | Tek | −0.00137463 | −0.00183633 | −0.00450633 | −0.00425658 | −0.00274789 | 0 | 0 |
| 100201 | Tmem64 | 7.08E−05 | 0.002417587 | −0.00246933 | 0.000204817 | −0.00068686 | 0 | 0 |
| 22004 | Tpm2 | 0.000159136 | 0.001798098 | 0.000912744 | 0.001318088 | 0.001358863 | 0 | −0.21068374 |
| 330286 | D630045J12Rik | −0.00146435 | −0.00401069 | −0.00617445 | −0.00514911 | −0.00268261 | 0 | 0 |
| 12876 | Cpe | 0.001152931 | 0.003111286 | 0.006484206 | 0.004051901 | 0.004731303 | 0 | 0 |
| 170442 | Bbox1 | 0.001553103 | 0.000799376 | 0.004268819 | 0.00448722 | 0.002809428 | 0 | 0 |
| 20677 | Sox4 | −0.00270439 | −0.00243181 | −0.00448343 | −0.0037683 | −0.00139217 | 0 | 0 |
| 100043272 | Inafm2 | 0.001174041 | 0.002533383 | 0.006177139 | 0.005958229 | 0.003504868 | 0 | 0 |
| 67847 | Sncaip | −0.00110733 | 0 | −0.00429385 | −0.00407658 | −0.0032384 | 0 | 0 |
| 66058 | Tmem1768 | 0.002365378 | −0.00037831 | 0.003938949 | 0.003344302 | 0.004023694 | 0 | 0.072493171 |
| 67249 | Thcid19 | −0.00069209 | 0.000158364 | −0.00362178 | −0.00362164 | −0.00132247 | 0 | 0 |
| 56149 | Tamalin | −0.00047193 | 0.001090313 | −0.00376211 | −0.00452461 | −0.00238533 | 0 | 0 |
| 76915 | Mnd1 | 0.000565198 | −0.00261487 | 0.00306293 | 0.002632248 | 0.001545796 | 0 | 0 |
| 66311 | Cenpw | −0.00135885 | 0 | −0.00692812 | −0.00531454 | −0.00501206 | 0 | 0 |
| 54216 | Pcdh7 | 0.000292151 | −0.00024232 | 0.002719534 | 0.001522546 | 0.001148807 | 0 | −0.02620164 |
| 107476 | Acaca | 0.001019525 | −0.00169043 | 0.00125302 | 0.00105723 | 0.000922801 | 0 | 0.149868968 |
| 231986 | Jazf1 | −0.00282956 | 0 | −0.00544529 | −0.00534636 | −0.00252505 | 0 | 0 |
| 246707 | Emilin2 | 7.81E−05 | 0.000275639 | 0.000131917 | −0.00081019 | −0.00111199 | 0 | 0 |
| 70750 | Kdsr | 0.00077304 | −0.0002332 | 0.001492834 | 0.001428457 | 0.001865707 | 0 | 0 |
| 64817 | Svep1 | 0.001171748 | −0.00159639 | 0.001208014 | 0.002837213 | 0.002772911 | 0 | 0 |
| 66953 | Cdca7 | −0.00152616 | −0.00352162 | −0.00401885 | −0.00395349 | −0.00298382 | 0 | 0 |
| 12804 | Cntfr | 0.002496414 | 0.003938275 | 0.006595875 | 0.004919729 | 0.006328081 | 0 | 0 |
| 270624 | Spin4 | −0.00124987 | −0.0009548 | −0.00131681 | −0.00144629 | −0.00253285 | 0 | 0 |
| 15368 | Hmox1 | −5.45E−05 | 0.006134764 | −0.00337694 | −0.00149221 | 0.000571899 | 0 | 0 |
| 19378 | Aldh1a2 | 0.001992013 | −0.00120599 | 0.002067004 | 0.00209295 | 0.003332454 | 0 | 0 |
| 22390 | Wee1 | −0.00075698 | −0.00187374 | −0.00419058 | −0.00547182 | −0.00296801 | 0 | 0.165942926 |
| 96957 | Tmem62 | −0.00154497 | −0.00022765 | −0.00274643 | −0.00241272 | −0.00296841 | 0 | 0 |
| 108078 | Olr1 | 0.000765052 | 0.001867814 | 0.005273618 | 0.005468027 | 0.00221314 | 0 | 0 |
| 233833 | Tnrc6a | −0.00050672 | −0.00101199 | −0.00416199 | −0.00433726 | −0.00336637 | 0 | 0 |
| 20807 | Srf | 0.000357498 | −0.00053794 | 0.00443976 | 0.005215205 | 0.002820667 | 0 | 0 |
| 15458 | Hpx | −0.00095071 | 0.001170044 | −0.0048244 | −0.00218616 | −0.00291497 | 0 | 0 |
| 17286 | Meox2 | −0.00117838 | −0.001066 | −0.00432784 | −0.00440896 | −0.00236619 | 0 | 0 |
| 74735 | Trim14 | 0.001054089 | 0.004397511 | 0.003917266 | 0.003743575 | 0.003094593 | 0 | 0 |
| 12351 | Car4 | −0.00244907 | −0.00165527 | −0.00397542 | −0.00580906 | −0.00559409 | 0 | 0 |
| 11553 | Adra2c | −0.00119384 | −0.00157584 | −0.0049806 | −0.00575231 | −0.00031502 | 0 | 0 |
| 11423 | Ache | 0.001412248 | 0.001056703 | 0.002058244 | 0.002477812 | 0.003020447 | 0 | 0 |
| 20364 | Selenow | 0.001418914 | −0.00103592 | 0.0013609 | 0.002681756 | 0.001796637 | 0 | 0 |
| 19285 | Cavin1 | 0.00141809 | −0.00034899 | 0.002872385 | 0.002023415 | 0.000378652 | 0 | 0 |
| 20494 | Slc1062 | −0.00047278 | −0.00183229 | 7.72E−05 | −0.00197989 | −0.00082554 | 0 | −0.18465906 |
| 20612 | Siglec1 | 0.001461219 | −0.00125933 | 0.003737986 | 0.003353391 | 0.003147794 | 0 | 0.049014253 |
| 229906 | Gtf2b | 0.000660795 | 0.001897877 | 0.005485159 | 0.004789933 | 0.001794657 | 0 | 0 |
| 11810 | Apobec1 | 0.001602813 | 0.002257749 | 0.000739745 | 0.000645408 | 0.002412914 | 0 | 0 |
| 15505 | Hsph1 | −0.0023612 | −0.00494158 | −0.00703774 | −0.00775811 | −0.00637329 | 0 | 0 |
| 18792 | Plau | 0.001299286 | −0.00477303 | 0.00052645 | 0.001693399 | 0.000956168 | 0 | 0 |
| 11433 | Acp5 | −0.00161738 | −0.00095377 | −0.00745172 | −0.00622238 | −0.00429386 | 0 | 0 |
| 71436 | Flrt3 | −0.00157949 | −0.0010276 | −0.00418014 | −0.00286046 | 0.000523238 | 0 | 0 |
| 13058 | Cybb | 0.003680751 | 0.001336108 | 0.001989228 | 0.004908179 | 0.003878426 | 0 | 0 |
| 12654 | Chil1 | 0.001423755 | −0.00331361 | −0.00304251 | −0.0035517 | −0.00211971 | 0 | 0.150224056 |
| 14563 | Gdf5 | 0 | 0 | 0 | 0 | 0.008608882 | 0 | 0 |
| 18104 | Nqo1 | 0.003189657 | 0.001647984 | 0.00280369 | 0.003396559 | 0.002795696 | 0 | 0 |
| 14119 | Fbn2 | −0.00260281 | −0.00111023 | −0.00061623 | −0.00457319 | −0.00835914 | 0 | 0 |
| 20302 | Ccl3 | 0 | −0.00024018 | 0 | 0 | 0 | 0 | 0 |
| 26386 | Hsf4 | 0.002784373 | −0.00109294 | −0.00224044 | −0.00156465 | 0.000475891 | 0 | 0 |
| 21928 | Tnfaip2 | 0.003311073 | −0.00090368 | 0.004092369 | 0.003957557 | 0.003625482 | 0 | 0.081446655 |
| 27206 | Nrk | −0.00269535 | 0 | −0.00188354 | −0.00400077 | −0.00068036 | 0 | 0 |
| 68054 | Serpina1Z | 0.002582519 | −0.00455056 | −0.00339643 | −7.74E−05 | −0.00016302 | 0 | 0.09177871 |
| 12406 | Serpinh1 | −0.00347914 | 0.000949222 | −0.00479155 | −0.00512599 | −0.00388555 | 0 | 0 |
| 13040 | Ctss | 0.003399307 | −0.00164044 | 2.61E−06 | 0.003560894 | 0.00303477 | 0 | 0 |
| 14960 | H2-Aa | 0.001697286 | −0.0068001 | −0.00463286 | 0.001307793 | 0.000975064 | 0 | 0.266682084 |
| 94352 | Lox12 | −0.00258046 | 0.000681684 | 7.05E−05 | −0.00155977 | −0.00219498 | 0 | 0 |
| 217203 | Tmem106a | 0.002988538 | 0.004247288 | 0.001969902 | 0.004788697 | 0.004778207 | 0 | 0 |
| 50530 | Mfap5 | −0.00279458 | 0 | −0.00172449 | −0.00013834 | 0.00023661 | 0 | 0 |
| 230379 | Acer2 | 0.003635523 | 0.003529361 | 0.00616991 | 0.007002431 | 0.005988525 | 0 | −0.00431386 |
| 208691 | Eif5a2 | 0.002997308 | 0 | 0.001960019 | 0.004424819 | 0.003299348 | 0 | 0 |
| 14586 | Gfra2 | −0.00316941 | −0.0036422 | −0.00377025 | −0.00266725 | −0.00121716 | 0 | 0 |
| 22329 | Vcam1 | 0.003193627 | 0.003393944 | 0.002835078 | 0.000756741 | 0.000926565 | 0 | 0 |
| 12286 | Cacnala | 0.00198602 | −0.00141011 | 0.003728078 | 0.005782444 | 0.003575846 | 0 | 0 |
| 30963 | Hacd1 | −0.00265594 | −0.0036922 | −0.00380399 | −0.00567151 | −0.00260939 | 0 | 0 |
| 64293 | Stk32b | 0.002604627 | −0.00031111 | 0.001678172 | 0.003935154 | 0.004014489 | 0 | 0 |
| 13527 | Dtna | 0.002421229 | 0.00179788 | 0.001791775 | 0.00375965 | 0.001479842 | 0 | 0 |
| 12870 | Cp | 0.002609712 | −0.00042003 | −0.00093758 | 0.001224092 | 0.002981195 | 0 | 0 |
| 171543 | Bmf | −0.00154982 | 0.001162884 | 0.003540831 | 0.001822859 | −0.00114359 | 0 | 0 |
| 216725 | Adamts2 | −0.00237194 | −0.00020323 | −0.00548969 | −0.00202971 | −0.00313894 | 0 | 0 |
| 14128 | Fcer2a | −0.00355471 | −0.00030268 | −0.00386003 | −0.00736782 | −0.00365954 | 0 | 0 |
| 81799 | C1qtnf3 | −0.0030544 | 0.000505946 | −0.00267598 | −0.00357748 | −0.00299917 | 0 | 0 |
| 68709 | Cilp2 | −0.00271407 | 0.000378962 | −0.00119085 | −0.00268132 | −0.00246032 | 0 | 0 |
| 211550 | Tifa | 0.003160077 | 0.003981211 | 0.002685249 | 0.006098707 | 0.004807345 | 0 | 0 |
| 68591 | Mocos | 0.002265923 | 0.000225183 | 0.000294539 | 0.000698979 | 0.001973022 | 0 | 0 |
| 17916 | Myo1f | 0.002948391 | 0.000478269 | 0.001907644 | 0.003957776 | 0.003679739 | 0 | 0 |
| 64918 | Bhmt2 | 0.002694588 | 0.002088283 | 0.003219824 | 0.003977041 | 0.004023869 | 0 | 0 |
| 18830 | Pltp | 0.00225363 | 0.001150432 | −0.00034511 | −0.00083167 | 0.000562343 | 0 | −0.13471394 |
| 78688 | Nol3 | 0.002534778 | −0.00064059 | 0.001946362 | 0.001410885 | 0.001776408 | 0 | 0 |
| 20377 | Sfrp1 | 0.003182318 | −0.00335474 | 0.00663202 | 0.006362641 | 0.004716159 | 0 | 0 |
| 22032 | Traf4 | −0.00244104 | 0.000526519 | −0.00340362 | −0.00290162 | −0.00228126 | 0 | 0 |
| 14969 | H2-Eb1 | 0.001458782 | −0.00291857 | −0.00206426 | 0.00526199 | 0.004074658 | 0 | 0.014816177 |
| 69183 | C1qtnf2 | −0.00323142 | −0.00366663 | −0.00743364 | −0.00628147 | −0.00449298 | 0 | 0 |
| 246256 | Fcgr4 | 0.002822473 | −0.00059469 | 0.001597776 | 0.002786272 | 0.004422993 | 0 | 0 |
| 12828 | Col4a3 | −0.00189583 | 0.001187088 | −0.00334559 | −0.00210344 | −0.00274837 | 0 | 0 |
| 26399 | Map2k6 | −0.0022216 | 0.000706975 | 0.000526149 | 0.0009225 | −7.69E−05 | 0 | −0.06611293 |
| 233733 | Galnt18 | −0.00244054 | −0.00034183 | −0.00241998 | −0.00337563 | −0.00111594 | 0 | 0 |
| 14086 | Fscn1 | −0.00259453 | 0.001573856 | 0.000826443 | 0.001509139 | −0.00187468 | 0 | 0 |
| 15460 | Hr | −0.00247152 | −0.00101645 | −0.00253682 | −0.00465797 | −0.00445965 | 0 | 0 |
| 22240 | Dpysl3 | −0.00198291 | 0.003760736 | −0.00435387 | −0.00395074 | −0.00136294 | 0 | 0 |
| 14962 | Cfb | 0.003612112 | −5.01E−05 | 0.001376268 | 0.00176982 | 0.001333147 | 0 | −0.0528715 |
| 18478 | Pah | −0.00252608 | −0.00092325 | 0.002507662 | 0.002254321 | −0.00014007 | 0 | 0 |
| 13813 | Eomes | 0 | 0 | 0.006775818 | 0 | 0.003889393 | 0 | 0 |
| 12831 | Col5a1 | −0.00293668 | −0.00087724 | −0.00235393 | −0.0034712 | −0.0041748 | 0 | 0 |
| 18534 | Pck1 | −0.00278338 | −0.00105763 | −0.00248608 | −0.0030401 | −0.00292726 | 0 | 0 |
| 15370 | Nr4a1 | 0.002278796 | 0.002951091 | 0.001551726 | 0.003376761 | 0.006404357 | 0 | 0 |
| 338370 | Nalon | 0.002450832 | 0 | 0.002053331 | 0.00475318 | 0.004090466 | 0 | 0 |
| 246709 | Rgs13 | 0.002442321 | −0.00055512 | 0 | 0.003414295 | 0.001816185 | 0 | 0 |
| 233332 | Adamts17 | −0.00206937 | −0.00094924 | −0.00058046 | 0.001263415 | 0.000858664 | 0 | 0 |
| 69354 | Slc3884 | −0.00263897 | 0.001010158 | −0.00017947 | −0.00072754 | −0.00045074 | 0 | 0 |
| 15442 | Hpse | 0.002931297 | 0.00289412 | 0.002068427 | 0.005218564 | 0.004881359 | 0 | 0 |
| 16842 | Lef1 | −0.00259896 | −0.00263084 | −0.00480627 | −0.00730833 | −0.0067299 | 0 | 0 |
| 57914 | Crif2 | 0.002153957 | −0.0003379 | 0.002466699 | 0.000510284 | 0.002451897 | 0 | 0 |
| 22337 | Vdr | 0.003063591 | 0.002101455 | 0.003527421 | 0.004955619 | 0.005071818 | 0 | 0 |
| 22223 | Uchl1 | 0.002353937 | 0.000600058 | 0.003980496 | 0.005571842 | 0.002817494 | 0 | 0 |
| 22361 | Vnn1 | −0.00277671 | −0.00332955 | −0.0005718 | 0.000331652 | −0.00054972 | 0 | 0 |
| 209837 | Slc38a5 | −0.00317296 | −0.00578528 | −0.005585 | −0.00775905 | −0.00596558 | 0 | 0.523574694 |
| 210719 | Mkx | 0.001372175 | −0.00525508 | −0.00175628 | 0.001113497 | −0.00075505 | 0 | 0.13738479 |
| 58998 | Nectin3 | −0.00254947 | −0.00122062 | −0.004651 | −0.00489556 | −0.00601791 | 0 | 0 |
| 68774 | Ms4a6d | 0.002488412 | 0.004972273 | 0.006703876 | 0.005597945 | 0.004970245 | 0 | 0 |
| 109264 | Me3 | 0.001930139 | 0 | 6.49E−05 | 0.001308278 | 0.002592245 | 0 | 0 |
| 83965 | Enpp5 | 0.001581657 | 0.003200534 | 0.003116625 | 0.002749207 | 0.002770819 | 0 | 0 |
| 110784 | Nr3c2 | 0.002646035 | 0.000651655 | 0.002470577 | 0.003367531 | 0.003399381 | 0 | 0 |
| 16912 | Psmb9 | 0.002341137 | 0.003040606 | 0.001395228 | 0.003559144 | 0.003211428 | 0 | −0.00643197 |
| 108052 | Slc14a1 | 0.002615655 | 0 | 0.005640409 | 0.006204521 | 0.005377526 | 0 | 0 |
| 170677 | Cdhr1 | 0.002545316 | 0.002058799 | 0.005191337 | 0.005389804 | 0.005243763 | 0 | 0 |
| 16450 | Jag2 | 0.00223117 | 0.001672268 | 0.001254863 | 0.002753933 | 0.003786256 | 0 | 0 |
| 18073 | Nid1 | −0.00189966 | −0.00211351 | −0.00237922 | −0.00402457 | −0.00343221 | 0 | 0 |
| 12775 | Ccr7 | −0.00245223 | −0.00136493 | −0.00396634 | −0.00823509 | −0.00476414 | 0 | 0 |
| 17536 | Meis2 | 0.0023177 | 0.003702649 | 0.005556603 | 0.007056802 | 0.00367783 | 0 | 0 |
| 12696 | Cirbp | 0.00235218 | −0.00038581 | 0.001060137 | 0.00138963 | 0.002341447 | 0 | 0 |
| 17762 | Mapt | 0.002098174 | 0.003174645 | 0.002453514 | 0.002127907 | 0.002592156 | 0 | 0 |
| 16176 | Il1b | 0.00176215 | 0.002550824 | 0.00238105 | 0.002059757 | 0.000191442 | 0 | 0 |
| 19703 | Renbp | 0.002274673 | 0.001320645 | 0.00459707 | 0.006271671 | 0.004360433 | 0 | 0 |
| 21789 | Tfpi2 | −0.00210682 | −0.00411002 | −0.00462269 | −0.00700087 | −0.00478358 | 0 | 0 |
| 15904 | Id4 | 0.002434088 | 0.000509902 | 0.003534204 | 0.004028524 | 0.00417418 | 0 | 0 |
| 58226 | Cacna1h | 0.002402344 | 0.002554936 | 0.006620392 | 0.006410717 | 0.005541943 | 0 | 0 |
| 14127 | Fcer1g | 0.002481528 | −0.00024212 | 0.002317874 | 0.003009197 | 0.002832767 | 0 | 0 |
| 72290 | Lsm11 | −0.00232416 | 0.000459779 | −6.64E−05 | −0.00072036 | −0.00177496 | 0 | 0 |
| 50908 | C1s1 | 0.00230353 | 0.002506341 | 0.002310909 | 0.001222669 | 0.00133292 | 0 | 0 |
| 22186 | Uba52 | 0.003099439 | −0.0007973 | 6.52E−05 | 0.000432965 | 0.001615088 | 0 | 0 |
| 14130 | Fcgr2b | 0.002335185 | 0.001047103 | 0.004296781 | 0.005889614 | 0.005857681 | 0 | 0 |
| 21940 | Cd27 | −0.00184836 | −0.00174778 | −0.00290639 | −0.00443001 | −0.0037224 | 0 | 0 |
| 327766 | Tmem26 | −0.00255536 | 0.001869402 | −0.00017948 | 1.31E−05 | 0.00097167 | 0 | 0 |
| 20893 | Bhlhe40 | 0.002358042 | 0.001953778 | 0.007317508 | 0.006442344 | 0.006201308 | 0 | −0.07808182 |
| 68087 | Dcakd | −0.00208942 | 0.000733101 | −0.0008811 | −0.00076635 | 4.21E−05 | 0 | 0 |
| 18822 | Plod1 | −0.00216796 | 0.000135352 | −0.00701851 | −0.00642296 | −0.00306026 | 0 | 0 |
| 12121 | Bicd1 | 0.002269459 | 0.000256899 | 0.000736436 | 0.003808813 | 0.001405788 | 0 | 0 |
| 270097 | Vat1l | −0.0028401 | 0 | −0.00350919 | −0.00260063 | −0.00514302 | 0 | 0 |
| 433470 | AA467197 | 0.002986459 | 0.000654522 | 0.002447047 | 0.00348268 | 0.002727546 | 0 | 0 |
| 13057 | Cyba | 0.00217269 | −0.00027181 | 0.000467248 | −6.08E−05 | −4.28E−05 | 0 | 0 |
| 22352 | Vim | 0.002843129 | 0.000161815 | 0.005630343 | 0.004138286 | 0.004146767 | 0 | 0 |
| 11801 | Cd5l | 0.001662773 | −0.00266219 | 0.001197463 | 0.001673387 | 0.001248824 | 0 | 0 |
| 18584 | Pde8a | 0.002510068 | −0.0005074 | 0.001670442 | 0.003727054 | 0.004261118 | 0 | 0 |
| 231162 | Cytl1 | 0.002114099 | 0.000302751 | 0.002521445 | 0.004614994 | 0.003886715 | 0 | 0 |
| 18791 | Plat | 0.00084259 | 0.000242747 | 0.002310654 | 0.000795116 | 0.001634668 | 0 | 0 |
| 18113 | Namt | −0.00247353 | −0.0015877 | −0.00148474 | −0.00166911 | −0.00288032 | 0 | −0.16867996 |
| 11746 | Anxa4 | 0.002327689 | −0.00106 | 0.004459544 | 0.005293995 | 0.00518562 | 0 | 0 |
| 78134 | Lpar4 | −0.00301498 | −0.000534 | −0.0070105 | −0.00646445 | −0.00418497 | 0 | 0 |
| 17930 | Myom2 | 0.003207611 | 0.000746037 | 0.004461571 | 0.004973903 | 0.004073811 | 0 | 0 |
| 66124 | Josd2 | −0.00229085 | −0.00106226 | −0.00307669 | −0.00245752 | −0.00302978 | 0 | 0.02375717 |
| 16514 | Kenj11 | −0.00289379 | 0 | −0.00465109 | −0.00515258 | −0.00288607 | 0 | 0 |
| 67426 | Coq8a | 0.001675945 | −0.0001939 | 0.002143572 | 0.00295218 | 0.002669926 | 0 | 0 |
| 16004 | Igf2r | 0.002041726 | −0.00168817 | 0.001007547 | 0.002406525 | 0.002597084 | 0 | 0 |
| 16582 | Klfc3 | 0.00186778 | −0.00015251 | 0.000334832 | 0.000861851 | 0.000430695 | 0 | 0 |
| 67608 | Narf | −0.00186353 | −0.00126369 | −8.50E−05 | −0.00077856 | −0.00014303 | 0 | 0 |
| 20167 | Rtn2 | 0.00290005 | 0.001361272 | 0.001333804 | 0.001978762 | −0.00057921 | 0 | 0 |
| 76884 | Cyfip2 | 0.002129958 | −0.00190278 | 0.001710694 | 0.002817405 | 0.001420615 | 0 | 0.076640643 |
| 19652 | Rbm3 | 0.001829677 | −7.48E−05 | 0.000586512 | 0.00116416 | 0.002649138 | 0 | 0.174022807 |
| 67198 | Spats2l | −0.00289266 | −0.0028031 | −0.00475035 | −0.0044588 | −0.0028247 | 0 | 0 |
| 64075 | Smoc1 | 0.002752645 | 0.001529538 | 0.000857106 | 0.002188584 | 0.001980942 | 0 | 0 |
| 333789 | N4bp2 | −0.00208487 | −0.00143728 | −1.57E−05 | −0.00144229 | −0.0009524 | 0 | 0 |
| 338403 | Cndp1 | 0.001743407 | 0.002496757 | 0.002204686 | 0.003057683 | 0.001355166 | 0 | −0.00918639 |
| 14314 | Fstl1 | −0.00204058 | −0.00224484 | −0.00607377 | −0.00584528 | −0.00183689 | 0 | 0 |
| 12142 | Prdm1 | 0.001222245 | −0.00167168 | 0.002533905 | 0.003623125 | 0.00368258 | 0 | 0 |
| 242274 | Lrrc7 | −0.00237292 | −0.00107549 | −0.00200679 | −0.00324235 | −0.002515 | 0 | 0 |
| 16918 | Myd | −0.00267591 | 0.000581526 | 0.000127949 | −0.00230891 | −0.00274387 | 0 | 0.14170673 |
| 22174 | Tyro3 | −0.00163241 | −0.002449 | −0.00580067 | −0.00638423 | −0.00544941 | 0 | 0 |
| 327814 | Ppfia2 | −0.00213103 | 0 | −0.00335824 | −0.00424078 | −0.00275364 | 0 | 0 |
| 106389 | Eaf2 | 0.002444157 | 1.29E−05 | 0.002297014 | 0.003049779 | 0.002284529 | 0 | 0 |
| 16728 | L1cam | 0.001837122 | −0.0009027 | 0.004737019 | 0.005349965 | 0.003732921 | 0 | 0 |
| 60411 | Cenpk | −0.00196136 | −0.00148945 | −0.00151304 | −0.00052284 | −0.00180292 | 0 | 0 |
| 319660 | Agmo | 0.001902919 | 0.001486655 | 0.001558333 | 0.004903584 | 0.00253497 | 0 | 0 |
| 55987 | Cpxm2 | 0.001595064 | −0.00412056 | −0.00084944 | 0.001767925 | 0.001648242 | 0 | 0.105239034 |
| 69066 | 1810010H24Rik | 0.002397638 | 0.002826435 | 0.004155799 | 0.006380491 | 0.003418451 | 0 | −0.01819254 |
| 69656 | Pir | 0.002598487 | −0.00018567 | 0.002259019 | 0.002699233 | −0.00020695 | 0 | 0 |
| 50794 | Klf13 | −0.001583 | −0.0005562 | −0.00273813 | −0.00248981 | −0.00377145 | 0 | 0 |
| 107503 | Atf5 | 0.002934464 | −0.00147624 | 0.002587555 | 0.002876661 | 0.003416011 | 0 | −0.10957235 |
| 13139 | Dgka | −0.00205575 | −4.61E−05 | −0.00156813 | −0.00114168 | −0.00113114 | 0 | 0 |
| 329278 | Tnn | −0.00179325 | −0.00180225 | −0.00212014 | −0.002713 | −0.00212775 | 0 | 0 |
| 18074 | Nid2 | −0.0019958 | 0.0016737 | 0.001223323 | −0.00151922 | −0.00142991 | 0 | 0 |
| 20317 | Serpinf1 | −0.00202476 | −0.00017167 | −0.00342978 | −0.00446616 | −0.00396939 | 0 | 0 |
| 20135 | Rrm2 | −0.00143774 | −0.00012168 | −0.00261203 | −0.00333835 | −0.00233735 | 0 | 0 |
| 207592 | Tbc1d16 | −0.00209094 | −0.00016981 | −0.00013673 | −0.00117794 | −0.00125644 | 0 | 0 |
| 20390 | Sftpd | −0.00157207 | 0.001363431 | −0.0007307 | −0.00249629 | −0.00079881 | 0 | 0.002701488 |
| 435766 | Tnni3k | 0 | 0 | 0 | 0 | 0 | 0 | 0 |
| 17754 | Map1a | 0.001451577 | 0 | 0.004400423 | 0.001779479 | 0.001260651 | 0 | 0 |
| 102278 | Cpne7 | 0.001892059 | 0 | 0.001373325 | 0.002301841 | 0.002011703 | 0 | 0 |
| 71911 | Bdh1 | −0.00382226 | −0.00205066 | −0.00343193 | −0.00890771 | −0.00662707 | 0 | 0 |
| 83771 | Tas1r3 | −0.00172013 | −0.00015068 | −0.00242921 | −0.00327962 | −0.00296795 | 0 | 0 |
| 53415 | Htatip2 | 0.002487565 | 0.000788024 | 0.000937693 | 0.002198555 | 0.001994555 | 0 | 0 |
| 71665 | Fuca1 | 0.002270733 | 0.001748408 | 0.004543168 | 0.004736826 | 0.005051409 | 0 | 0 |
| 16408 | Itgal | 0.00201652 | −0.00184358 | −0.0002029 | 0.006743339 | 0.003263107 | 0 | 0 |
| 74519 | Cyp2j9 | 0.00184732 | 0.001571245 | 0.000993098 | 0.00277542 | 0.002257071 | 0 | 0 |
| 12834 | Col6a2 | −0.00220726 | −0.00074542 | −0.00197435 | −0.00207476 | −0.00161473 | 0 | 0 |
| 17988 | Ndrg1 | 0.001867161 | −0.00127827 | 0.000885711 | 0.0020909 | −0.00304316 | 0 | 0.15487242 |
| 17755 | Map1b | 0.002203873 | 0.000377355 | −0.00025794 | 0.000808179 | −0.00147765 | 0 | 0 |
| 245595 | Zfp711 | −0.00176471 | −0.00032983 | −0.00471112 | −0.00272315 | −0.00300149 | 0 | 0 |
| 232087 | Mat2a | −0.00215136 | −0.00156535 | −0.00189681 | −0.00211099 | −0.00360609 | 0 | 0 |
| 17112 | Tm4sf1 | 0.002390768 | 0.000289883 | 0.006435495 | 0.00708728 | 0.002773204 | 0 | 0 |
| 16797 | Lat | 0.001907699 | 0.001849975 | 0.000561419 | 0.001324071 | 0.003008553 | 0 | 0 |
| 208869 | Dock3 | 0.001884345 | 0.000151191 | 0.001914618 | 0.004467072 | 0.001734198 | 0 | 0 |
| 108153 | Adamts7 | −0.00348701 | −6.49E−05 | −0.002113 | −0.00435151 | −0.00259466 | 0 | 0 |
| 17181 | Matn2 | −0.00152712 | 0.000475397 | 0.002744157 | 0.00353248 | −0.00129845 | 0 | 0 |
| 268880 | Xxylt1 | 0.002134101 | −0.00053927 | 0.001426446 | 0.001893621 | 0.002641621 | 0 | 0 |
| 93761 | Smarca1 | −0.00183945 | −0.00104509 | −0.0027241 | −0.0029203 | −0.00182455 | 0 | 0 |
| 12323 | Camk2b | 0.002375156 | 0.003636406 | 0.005958955 | 0.008029637 | 0.00613911 | 0 | −0.0078378 |
| 432628 | Mfsd2b | 0.002037036 | 0.001566964 | 0.003570171 | 0.004149957 | 0.003261033 | 0 | 0 |
| 75906 | Fam184a | 0.001793338 | 0.001530125 | 0.003230747 | 0.00260684 | 0.002293503 | 0 | 0 |
| 17150 | Mfap2 | −0.00161092 | −0.00046219 | 0.002042753 | 0.001671518 | 0.000365191 | 0 | 0 |
| 268595 | D430019H16Rik | −0.00238675 | −0.00050757 | −0.00265824 | −0.00260117 | −0.00250625 | 0 | 0 |
| 66107 | Widc21 | 0.004215926 | 0.005444433 | 0.007219277 | 0.009706429 | 0 | −0.15720011 | −0.65525055 |
| 338365 | Slc41a2 | 0.002613149 | 0.002468873 | 0.001803436 | 0.00351739 | 0.002566549 | 0 | 0 |
| 22637 | Zap7O | 0.002355698 | 0.004764373 | −8.54E−05 | 0.000469269 | 0.003925286 | 0 | 0 |
| 114564 | Csprs | 0 | 0.005021571 | 0 | 0 | 0 | 0 | 0 |
| 13048 | Cux2 | 0.002426364 | 0.00371942 | 0.001041115 | 0.005922542 | 0.001730469 | −0.22701175 | −0.45134901 |
| 192201 | Wfdc15b | 0.002504949 | 0.007315571 | 0.001940231 | 0.003184537 | 0 | 0 | −0.21349818 |
| 11459 | Acta1 | −0.00218072 | −0.00431338 | −0.0017119 | −0.00059413 | −0.00365594 | 0 | 0.02127162 |
| 19175 | Psmb6 | −0.00208321 | −0.00163948 | −0.00228992 | −0.00391754 | −0.00152131 | 0 | 0.146653289 |
| 243881 | Cyp2b23 | 0 | −0.00029379 | 0 | 0 | 0 | 0 | 0.035545723 |
| 13077 | Cyp1a2 | −0.0019531 | −0.00105326 | 0.000895684 | 0.000623835 | −0.00088695 | 0 | 0.093376876 |
| 108112 | Eif4ebp3 | −0.00117291 | −0.00682895 | −0.0058866 | −0.00522028 | −0.00439806 | 0 | 0.291918534 |
| 18606 | Enpp2 | −0.00292807 | −0.00730285 | −0.00633042 | −0.00709695 | −0.00624023 | 0 | 0.530826741 |
| 13706 | Cela2a | −0.00174619 | −0.00012653 | 0.001667793 | 0.000935062 | 0 | 0 | 0.098062737 |
| 56620 | Clec4n | 0.0021183 | 0.00151173 | −0.00042837 | −0.00226343 | −0.00096111 | 0 | −0.20508499 |
| 277753 | Cyp4a12a | 0 | −0.00545663 | −0.00066725 | 0 | 0 | 0 | 0.286970376 |
| 66786 | Olfr701 | 0 | 0.003592863 | 0.002210244 | 0 | 0 | 0 | 0 |
| 74147 | Ehhadh | −0.0013472 | −0.00213489 | −0.00274379 | −0.00259682 | −0.00177478 | 0 | 0.287156882 |
| 667278 | Mup-ps16 | 0 | −0.00603514 | 0 | 0 | 0 | 0 | 1.184152613 |
| 20714 | Serplna3k | 0.00301694 | 0.002927561 | 0.00565546 | 0.006838539 | 0 | −0.9037581 | −0.33275401 |
| 57349 | Ppbp | 0.003049478 | 0.001792218 | 0.001516645 | 0.00181364 | 0.004179817 | 0 | 0.086294038 |
| 25908 | Eif2s3y | 0.000839165 | 0.00083849 | −0.00364576 | −0.00394922 | −0.00127621 | 0 | 0 |
| 107141 | Cyp2c50 | −0.00289088 | −0.00393195 | −0.0076747 | −0.00663752 | 0 | 0 | 0 |
| 16891 | Lipg | −0.00253817 | −0.00578308 | −0.00424149 | −0.0056825 | −0.00231907 | 0 | 0.342846283 |
| 12346 | Car1 | −0.00203168 | −6.20E−05 | −0.00175816 | −0.00559682 | −0.00501178 | 0 | 0.248930129 |
| 13982 | Esr1 | 0.002503377 | 0.003881236 | 0.005858616 | 0.009215797 | 0.004978495 | 0 | 0 |
| 14456 | Gas6 | 0.001854884 | 0.002810844 | −0.00278367 | −0.00162986 | 0.001421374 | 0 | −0.08839329 |
| 192200 | Wfdc12 | 0 | 0.005630452 | 0 | 0 | 0.003990929 | 0 | −0.29303112 |
| 108995 | Tbc1d10c | 0.001673225 | 2.81E−05 | 0.002425195 | 0.00175147 | 0.00206192 | 0 | 0 |
| 13089 | Cyp2b13 | −0.001524 | −0.00304376 | −0.005358 | −0.0036981 | 0 | 0 | 0.395801552 |
| 53412 | Ppp1r3c | 0.002387784 | 0.002542508 | −0.00185815 | −0.00319704 | 0.000769558 | 0 | −0.1218497 |
| 18054 | Ngp | 0 | 0.006457491 | 0.007765983 | 0 | 0 | 0 | 0 |
| 58860 | Adamdec1 | 0.001104073 | 0.000871545 | −0.00211963 | −0.00166568 | −0.00065839 | 0 | 0.123041276 |
| 14468 | Gbp2b | −0.00147126 | −0.00471733 | −0.00101074 | −0.00097781 | −0.00346752 | 0 | 0 |
| 81489 | Dnajb1 | −0.00216515 | −0.00233635 | −0.00372551 | −0.00397252 | −0.00478357 | 0 | 0 |
| 13088 | Cyp2b1O | −0.00095197 | −0.00549223 | −0.00377285 | −0.00283917 | 0 | 0 | 0.198865997 |
| 14600 | Ghr | 0.002282863 | 0.002122742 | 0.003219567 | 0.004267465 | 0.002743259 | 0 | 0 |
| 18406 | Orm2 | 0 | 0.001378519 | 0.002196477 | 0 | 0 | 0 | 0 |
| 71754 | Cyp2d4O | 0 | −0.00472864 | 1.65E−05 | 0 | 0 | 0 | 0.341838049 |
| 66175 | Mustn1 | 0.001319784 | 0.001028836 | −2.67E−05 | −0.00074667 | −0.00036885 | 0 | 0 |
| 80890 | Trim2 | −0.00050228 | −0.00405961 | −0.00025091 | −0.00234056 | −0.00157372 | 0 | 0 |
| 20216 | Acsm3 | −0.00093177 | −0.00473754 | 0.005444044 | 0.004905522 | 0.000983495 | 0 | 0 |
| 93842 | Igsf9 | −0.00151701 | −0.00456694 | −0.00453236 | −0.00605772 | −0.00253012 | 0 | 0 |
| 17773 | Mtnr1a | 0.002301198 | 0.001241164 | 0.001071103 | 0.001678198 | 2.69E−05 | 0 | 0 |
| 100689 | Spon2 | 0.001159237 | 0.003420092 | 0.00461038 | 0.004859503 | 0.002348065 | 0 | −0.03383838 |
| 20720 | Serplne2 | 0.002224333 | 0.00329653 | 0.005315371 | 0.005668602 | 0.002129283 | 0 | 0 |
| 107995 | Cdc2O | −0.00116933 | −0.00120007 | −0.00288566 | −0.00275146 | −0.0008252 | 0 | 0 |
| 27047 | Omd | 0.00209523 | −0.00167237 | −0.00092934 | −0.00162497 | 0.000838901 | 0 | 0 |
| 433904 | Ociad2 | −0.00085762 | −0.00550547 | −0.00075158 | −1.40E−05 | 0.000522982 | 0 | 0.198337489 |
| 50490 | Nox4 | −0.00212145 | −0.00296836 | −0.00256565 | −0.00453973 | −0.00522502 | 0 | 0 |
| 74488 | Lrrc15 | 0.002249235 | 0.002932933 | 0.002843858 | 0.003090232 | 0.004371418 | 0 | −0.07789694 |
| 20305 | Ccl6 | 0.001476211 | 0.003756065 | 0.003999816 | 0.000538287 | 0.003116295 | 0 | 0 |
| 195733 | Grhl1 | −0.00080551 | −0.00513374 | −0.00084007 | −0.00406553 | −0.00208687 | 0 | 0.017127269 |
| 100559 | Ugt2b3B | 0 | −0.00406053 | −0.00484155 | 0 | 0 | 0 | 0 |
| 213056 | Fam126b | −0.00135792 | −0.00275731 | −0.00205606 | −0.00243716 | −0.00269522 | 0 | 0.014806079 |
| 14262 | Fmo3 | 0.000242837 | −0.00258129 | −0.00037793 | 0.000863895 | −0.00127989 | 0 | 0 |
| 69099 | 1810009N02Rik | 0 | −0.00303694 | 0 | 0 | 0 | 0 | 0 |
| 12389 | Cav1 | −0.00218868 | −0.00402113 | −0.00423079 | −0.00676881 | −0.00176321 | 0 | 0.039000012 |
| 319278 | Shfl | 0.002124803 | 0.001109874 | 0.001008312 | 0.001758277 | 5.00E−05 | 0 | 0 |
| 11858 | Rnd2 | 0.001399043 | 0.004925211 | 0.005986753 | 0.002972388 | 0.004272599 | 0 | 0 |
| 13992 | Khdrbs3 | −0.00138137 | −0.00355052 | −0.0025065 | −0.00527637 | −0.00021388 | 0 | 0 |
| 13214 | Defb1 | 9.49E−05 | −0.00309806 | −0.00037131 | −0.00087891 | 0 | 0 | 0 |
| 18793 | Plaur | 0.001324411 | −0.0001698 | 0.001974737 | 0.000421345 | 0.001227402 | 0 | 0 |
| 53877 | Ear4 | 0 | −0.00164665 | 0 | 0 | 0 | 0 | 0 |
| 17864 | Mybl1 | 0.00215558 | 0.003521189 | −0.00208906 | 0.000116126 | 0.001419563 | 0 | −0.05692165 |
| 22784 | Slc30a3 | 0.001840053 | 0.003763149 | 0 | 0.001413201 | 0.001638478 | 0 | 0 |
| 269589 | Sytl1 | 0.000711345 | 0.002646649 | 0.000395309 | 0.000307987 | 0.000502656 | 0 | −0.20760729 |
| 633640 | Tmem267 | −0.0020704 | −0.00151708 | −0.00062987 | 0.000617432 | 0.000916756 | 0 | 0 |
| 15486 | Hsd17b2 | −0.00044904 | −0.00299008 | −0.00382582 | −0.00239383 | −0.0004509 | 0 | 0 |
| 17836 | Mug1 | −0.00252508 | −0.00349531 | −0.00460549 | −0.00414948 | −0.00264872 | 0 | 0 |
| 238257 | Tmem30b | 0.001016096 | 0.004191673 | 0.000499263 | 0.002210023 | 0.002469332 | 0 | 0 |
| 67896 | Ccdc80 | −0.00177545 | −0.00159081 | −0.0029942 | −0.00256377 | −0.00251521 | 0 | 0 |
| 66442 | Spc25 | −0.00200655 | −0.00459981 | −0.00500002 | −0.00074581 | −0.00126147 | 0 | 0.130023959 |
| 17691 | Slk1 | 0.00141475 | 0.001991846 | −0.00085774 | 8.06E−05 | 0.001945838 | 0 | 0 |
| 22249 | Unc13b | 0.001543108 | 0.002563076 | −0.00156963 | −0.00223707 | −0.00141245 | 0 | −0.02115978 |
| 64296 | Abhd8 | −0.00133322 | −0.00268471 | −0.00210175 | −0.00247277 | −0.00218118 | 0 | 0.01821234 |
| 63873 | Trpv4 | 0.00109881 | −0.00377608 | −0.00325166 | 0.000426135 | −0.00105219 | 0 | 0 |
| 102022 | Ces2a | −0.00049156 | −0.00352966 | −0.00323368 | −0.00343153 | 0 | 0 | 0 |
| 231238 | Sel1l3 | −0.00091865 | −0.00205069 | −0.00135391 | −0.00159356 | −0.00417546 | 0 | 0 |
| 20848 | Stat3 | 0.002410974 | 0.003675141 | 0.003301874 | 0.005275784 | 0.003159414 | 0 | 0 |
| 13034 | Ctse | 0.002845662 | 0.002722211 | 0.004374585 | 0.006250822 | 0.004769423 | 0 | 0 |
| 100504404 | H2-Ea | 0 | −0.00586581 | 0 | 0 | 0 | 0 | 0.00690075 |
| 223453 | Dap | 0.001744389 | 0.002396622 | 0.001764052 | 0.00397192 | 0.002071584 | 0 | 0 |
| 72042 | Cotl1 | 0.001166009 | −0.00032436 | −0.00204896 | −0.00246158 | −0.00204039 | 0 | 0 |
| 71974 | Prmt3 | 0.001064746 | 0.004868903 | 0.003688685 | 0.003940949 | 0.002350037 | 0 | 0 |
| 229759 | Olfm3 | 0.000781446 | 0.001524838 | −0.00054911 | 0.000282712 | 0.000579128 | 0 | 0 |
| 23985 | Slc26a4 | 0.001436433 | −0.00049737 | −0.0025851 | −0.00267957 | −0.00117193 | 0 | 0 |
| 107449 | Unc5b | 0.001145645 | 0.003588062 | 0.001146619 | 0.003339862 | −0.00021453 | 0 | 0 |
| 71833 | Dcaf7 | 0.000937678 | 0.00238157 | −0.00129526 | −0.00155487 | −0.00072597 | 0 | 0 |
| 30937 | Lmcd1 | 0.001476807 | 0.001253115 | −0.00107244 | −0.00128037 | 0.000669749 | 0 | 0 |
| 66589 | Ube2v1 | 0.002327676 | 5.09E−05 | −0.00055107 | −0.00033801 | −0.00047073 | 0 | 0 |
| 76886 | Fam81a | −0.00101793 | −0.00355772 | −0.00758075 | −0.006757 | −0.00330514 | 0 | 0.195440848 |
| 18778 | Pla2g1b | 0 | −0.00270956 | 0 | 0 | −0.00140184 | 0 | 0 |
| 14537 | Gcnt1 | −1.77E−05 | −0.004739 | −0.00271061 | −0.00274562 | −0.00047482 | 0 | 0 |
| 434674 | Slc22a28 | 0 | 0.002338284 | 0 | 0 | 0 | 0 | 0 |
| 21828 | Thbs4 | 0.00188463 | 0.003244972 | −0.0008791 | 0.00168065 | 0.000853799 | 0 | 0 |
| 66607 | Ms4a4d | 0.000996422 | 0.002503271 | 0.006268533 | 0.006288092 | 0 | 0 | 0 |
| 94190 | Ophn1 | −0.00051952 | −0.00222108 | −0.00192049 | −0.00185699 | −0.00060457 | 0 | 0 |
| 12721 | Coro1a | 0.000699614 | −0.00015947 | −0.00162003 | 0.00052648 | −0.00175946 | 0 | 0 |
| 230162 | Zfp189 | −0.00162356 | −0.00323584 | −0.00178721 | −0.00295999 | −0.0046827 | 0 | 0.184076412 |
| 12722 | Clca3a1 | 0.001195218 | 0.006414039 | 0.004946797 | 0.00691973 | 0 | 0 | 0 |
| 12370 | Casp8 | −0.00172658 | −0.00299813 | −0.00409263 | −0.0009357 | −0.00049769 | 0 | 0.018813088 |
| 19733 | Rgn | −0.00075961 | −0.00234382 | 0.003694283 | 0.002222999 | 0.002043003 | 0 | 0 |
| 21916 | Tmod1 | 0.000102547 | 0.002284666 | −0.00286593 | −0.00183641 | −0.00194206 | 0 | 0 |
| 12297 | Cacnb3 | −0.0010446 | 0.00034041 | 0.000467485 | −0.00087935 | −0.00231985 | 0 | 0 |
| 170639 | Olfr78 | −0.00167891 | −0.0026966 | 0.001103043 | 0.000350458 | −0.00079392 | 0 | 0 |
| 57294 | Rps27 | −0.00091572 | −0.00292938 | −0.00222516 | −0.00297265 | −0.00114266 | 0 | 0 |
| 71907 | Serpina9 | 0 | 0.00593306 | 0 | 0 | 0.004147035 | 0 | 0 |
| 20725 | Serpinb8 | 0.000425132 | 0.002314791 | −0.00226544 | −0.00013178 | −0.00057661 | 0 | 0 |
| 71684 | Rbm43 | 0.000312246 | 0.001624096 | 0.001663057 | −0.00017752 | −0.00085531 | 0 | 0 |
| 16612 | Klk1 | 0 | −0.00279076 | −0.00281375 | 0 | 0 | 0 | 0 |
| 233005 | Cyp2a22 | 0 | −0.0050018 | 0 | 0 | 0 | 0 | −0.0847112 |
| 67867 | Lrrc28 | 0.000272208 | −0.00274787 | 0.000948035 | 0.000647329 | 0.000393887 | 0 | 0 |
| 11655 | Alas1 | −0.00185007 | 0.001624161 | 0.001951662 | 0.001497215 | −7.39E−05 | 0 | −0.11389289 |
| 50850 | Spast | −0.00159377 | −0.00279413 | −0.00161296 | −0.00254568 | −0.0012063 | 0 | 0 |
| 11857 | Arhgdlb | 0.001209783 | 0.002716696 | 0.003050855 | 0.005125793 | 0.002805991 | 0 | 0 |
| 17313 | Mgp | 0.000577527 | −0.00380455 | −0.00034865 | −0.00217568 | −0.00163616 | 0 | 0 |
| 26451 | Rpl27a | −0.00143312 | −0.00272774 | −0.00071897 | −0.001693 | −0.00204753 | 0 | 0 |
| 17394 | Mmp8 | 0.001708859 | 0.003390812 | 0.00062318 | 0.001374381 | 0.000379588 | 0 | 0 |
| 18639 | Pfkfb1 | −0.00158775 | 0.000125054 | 0.000788093 | −0.00088863 | −0.00056763 | 0 | 0 |
| 319710 | Frmd6 | −4.37E−05 | −0.00560702 | −0.00408874 | −0.00488832 | −0.0018651 | 0 | 0 |
| 69165 | Cd209b | 0.000792908 | 0.002750291 | 0.001873269 | 0.002206504 | 0.00014893 | 0 | 0 |
| 13909 | Ces3b | 0 | 0.000318 | −0.00191593 | 0 | 0 | 0 | 0.269326545 |
| 209186 | Acnat2 | 0.001316749 | 0.004077543 | 0.00489603 | 0.005622608 | 0 | 0 | −0.20989275 |
| 20529 | Slc31a1 | −0.00239016 | −0.00139597 | −0.00167854 | −0.00290165 | −0.00114602 | 0 | 0.101175644 |
| 13190 | Dct | 0.002222477 | 0.002366506 | 0.005001956 | 0.002084742 | 0.004830733 | 0 | −0.21095754 |
| 108148 | Galnt2 | 0.000813488 | 0.001391545 | 0.000231578 | 0.000160829 | −0.000261 | 0 | −0.04020669 |
| 19683 | Rdh16 | 5.87E−05 | 0.003721368 | 0.002955052 | 0.002872326 | 0 | 0 | 0 |
| 15902 | Id2 | 0.000655473 | 0.001801737 | −0.00132985 | −0.00247973 | −0.00150225 | 0 | −0.03690574 |
| 622473 | Ripply1 | 0 | −0.00398305 | 0 | 0 | 0 | 0 | 0 |
| 50702 | Cfhr1 | 0.000648676 | 0.001921656 | −0.00055484 | −6.89E−05 | 0 | 0 | 0 |
| 223921 | Aaas | 0.000257841 | −0.00199133 | 0.005481185 | 0.004687165 | 0.00153743 | 0 | 0 |
| 70683 | Utp2O | 0.000655533 | 0.002928361 | 0.002092516 | 0.002649732 | 0.000386545 | 0 | 0 |
| 211666 | Mgst2 | −0.0014997 | −0.00289355 | −0.00428242 | −0.00342348 | −0.00097097 | 0 | 0 |
| 237459 | Cdk17 | 0.000732124 | 0.004161838 | 0.00146986 | 0.00200989 | 0.001077025 | 0 | −0.14955722 |
| 14420 | Galc | 0.001580836 | 0.000274676 | 0.001272943 | 0.000447319 | 0.001803065 | 0 | 0 |
| 235505 | Cd109 | −0.00023641 | −0.00098979 | 4.85E−06 | −0.00092702 | −0.00088609 | 0 | 0 |
| 54418 | Fmn2 | 0.002397406 | 0.001149008 | 0.000344092 | 0.000177068 | 0.001383037 | 0 | 0 |
| 15270 | H2ax | −0.00103329 | −0.00228453 | −0.00153882 | −0.00039837 | −0.00120913 | 0 | 0 |
| 94175 | Hrg | −0.0007944 | −0.00436903 | −0.00438476 | −0.00530383 | −0.00514858 | 0 | 0.069733071 |
| 14972 | H2-K1 | 0.002429569 | −0.00051311 | 0.003779922 | 0.002609153 | 0.003051452 | 0 | −0.1556199 |
| 207920 | Esrp1 | 0.000950889 | 0.001253308 | 0.001109328 | 0.002927994 | 0.002217276 | 0 | 0 |
| 11566 | Adss | 0.001981508 | −0.00016433 | −0.0015322 | −0.00053263 | 0.000625361 | 0 | 0 |
| 13360 | Dhcr7 | 0.000381555 | 0.001989993 | 0.001649115 | 0.002272485 | 0.00168278 | 0 | 0 |
| 68067 | Mrnip | 0.000986236 | 0.000748526 | 0.000397341 | 0.003343824 | 0.003189068 | 0 | 0 |
| 13074 | Cyp17a1 | 0.000409889 | −0.00123993 | −0.00207327 | −0.00018354 | −0.0010611 | −0.13640217 | 0 |
| 77318 | Ankrd55 | 0.001933158 | 0.00573434 | 0.005349164 | 0.005379993 | 0.003657433 | 0 | 0 |
| 258216 | Olfr1034 | 0 | −0.002591 | 0 | 0 | 0 | 0 | 0.102914341 |
| 268756 | Gulo | −0.00163057 | −0.00193438 | −0.00106121 | −0.00063642 | 0 | 0 | 0 |
| 16186 | Il2rg | 0.000671743 | 0.004319787 | −0.00248566 | −0.00129912 | 0.001253396 | 0 | 0 |
| 71729 | Rgs12 | 0.001249112 | 0.00167617 | 0.004629876 | 0.005543987 | 0.002433383 | 0 | 0 |
| 234673 | Ces2e | −9.48E−05 | −0.00489033 | −0.002052 | −0.00279891 | −0.00109359 | 0 | 0.48823682 |
| 76971 | Sult2a8 | −0.00212429 | −0.00207234 | −0.00455485 | −0.00303381 | 0 | 0.065776607 | 0.059730172 |
| 20660 | Sorl1 | 5.11E−05 | 0.002831399 | 0.001157616 | 0.000535025 | −0.000229 | 0 | 0 |
| 56739 | Rec8 | −0.00071848 | −0.00671683 | −0.00362181 | −0.00249016 | −0.00215026 | 0 | 0 |
| 20867 | Stip1 | −0.00163578 | −0.0010076 | −0.00211849 | −0.00299135 | −0.00270188 | 0 | 0 |
| 217258 | Abca8a | −0.00018653 | −0.00280409 | 0.001148197 | −0.00019256 | 0.000633369 | 0 | 0 |
| 216350 | Tspan8 | −0.00063188 | 0.002859606 | −0.00060266 | −0.00144489 | 0.000482348 | 0 | −0.07088061 |
| 12162 | Bmp7 | 0.001063997 | 0.002592558 | 0.000281267 | 0.005057242 | 0.004597282 | 0 | 0 |
| 12140 | Fabp7 | −0.00148617 | −0.00216136 | −0.00207989 | −0.00203355 | −0.00209496 | 0 | 0 |
| 319170 | H2ac22 | 0 | −0.00327588 | 0 | 0 | 0 | 0 | 0 |
| 380969 | Nckap5l | −0.00186648 | −0.00338581 | −0.00378364 | −0.00329663 | −0.00303593 | 0 | 0 |
| 74264 | Rnf138rt1 | 0 | 0.003600688 | 0 | 0 | 0 | 0 | 0 |
| 19361 | Rad51 | −0.0016408 | 0.000100535 | 0.000471875 | 0.000937514 | 0.00025314 | 0 | 0 |
| 381530 | Mup20 | 0 | 0.00039131 | 0 | 0 | 0 | 0 | −0.36926554 |
| 74103 | Nebl | 0.000984785 | 0.00193383 | 0.000454275 | 0.000724649 | 0.00403969 | 0 | 0.031973844 |
| 109624 | Cald1 | 0.001362642 | 0.001462268 | 0.003240715 | 0.00350303 | 0.001408478 | 0 | 0 |
| 76415 | Fam187b | −0.00081209 | −0.00151442 | −0.0025503 | −0.00265048 | −0.00212191 | 0 | 0.166300954 |
| 12298 | Cacnb4 | 0.002438294 | 0.001953798 | 0.00023552 | 0.003724518 | 0.003040648 | 0 | 0 |
| 78070 | Cpt1c | −0.00080344 | −0.00229648 | −0.00012984 | 0.000570486 | 0.000443189 | 0 | 0 |
| 53625 | B3gnt2 | 0.001739204 | 0.002492647 | 0.00666787 | 0.005006798 | 0.003803047 | 0 | 0 |
| 18171 | Nr1i2 | −0.00095622 | −0.00456658 | −0.00197074 | −0.00145144 | −0.00041759 | 0 | 0 |
| 13124 | Cyp8b1 | 0.000226713 | −0.00514145 | −0.00594173 | −0.00248054 | −0.0034208 | 0 | 0.212289789 |
| 233870 | Tufm | −0.0010877 | −0.00221063 | −0.00040371 | −0.00019993 | 0.000384045 | 0 | 0 |
| 12768 | Ccr1 | 0.002173775 | 0.003982082 | 0.006079753 | 0.007364239 | 0.004651453 | 0 | 0 |
| 74868 | Tmem65 | 0.000579919 | 0.001655588 | 0.000580684 | 5.99E−05 | −4.93E−05 | 0 | 0 |
| 16420 | Itgb6 | 0.00038166 | 0.002983171 | −0.00156652 | −0.00139521 | −0.00093297 | 0 | 0 |
| 66447 | Mgst3 | 0.001069154 | 0.002096377 | 0.000353865 | −0.00041703 | −0.00073191 | 0 | −0.18672379 |
| 22024 | Crisp2 | 0 | 0.002496713 | 0.003638503 | 0 | 0.002897013 | 0 | 0 |
| 74145 | F13a1 | 0.003024611 | 0.002517509 | 0.005189028 | 0.004042066 | 0.002019691 | 0 | 0 |
| 96875 | Prg4 | 0.002691959 | 0.000127786 | 0.002400987 | 0.00190275 | 0.00032599 | 0 | 0 |
| 69585 | Hjv | −0.00189243 | −0.00378061 | −0.00467789 | 0.000551778 | −0.00073744 | 0 | 0 |
| 13506 | Dsc2 | −0.00115708 | 0.000189087 | −0.00125683 | 0.001241466 | 0.001823757 | 0 | 0 |
| 12035 | Bcat1 | −0.0018276 | −0.00011833 | −0.00535367 | −0.00587267 | −0.003301 | 0 | 0 |
| 14778 | Gpx3 | 0.002404496 | −0.00307306 | −0.00489307 | −0.0044089 | 0.001617808 | 0 | 0 |
| 105859 | Csdc2 | −0.00282323 | −0.00205777 | −0.00222428 | −0.00469821 | −0.00182188 | 0 | 0 |
| 76905 | Lrg1 | 0.001957938 | −0.00033876 | 0.005730349 | 0.003621879 | 0.005356613 | 0 | 0 |
| 26364 | Adgre5 | 0.002479898 | 0.001088476 | 0.004654312 | 0.002100295 | 0.001445529 | 0 | 0 |
| 59095 | Fxyd6 | 0.002367481 | −0.00137214 | 0.002771602 | 0.001517215 | 0.002107043 | 0 | 0 |
| 11517 | Adcyap1r1 | 0.002184089 | 0.000660047 | 0.001573987 | 0.004043641 | 0.001717725 | 0 | 0 |
| 52882 | Rgs7bp | −0.00270501 | 0 | −0.00296658 | −0.00352074 | −0.00190456 | 0 | 0 |
| 22354 | Vipr1 | −0.00238956 | −0.00174522 | −0.00246014 | −0.00199258 | −0.00113423 | 0 | 0 |
| 14718 | Got1 | 0.001955889 | 0.000201193 | 0.004125174 | 0.005205811 | 0.002598977 | 0 | 0 |
| 22142 | Tubala | −0.00256917 | −0.00111167 | −0.0035136 | −0.0035743 | −0.00586727 | 0 | 0 |
| 69816 | Mzb1 | 0.002410246 | 0.001477636 | 0.001840842 | 0.000368787 | 0.002568177 | 0 | −0.06407666 |
| 19417 | Rasgrfl | 0.001810566 | −0.00072502 | 0.004096259 | 0.001872914 | 0.001981026 | 0 | 0 |
| 78925 | Srd5a1 | −0.00072903 | −0.0026256 | −0.00235107 | −0.00097467 | −0.00153371 | 0 | 0.554124297 |
| 70350 | Basp1 | −0.00235537 | −0.00188506 | −0.00351941 | −0.00439756 | −0.00249517 | 0 | 0 |
| 192190 | Pkhd1l1 | −0.00210625 | −0.00411996 | −0.00393687 | −0.00297312 | −0.00198391 | 0 | 0 |
| 17294 | Mest | −0.00166375 | 0.001713565 | 0.003759748 | 0.003303484 | 0.002205614 | 0 | 0 |
| 69675 | Pxdn | −0.00219392 | 0.00101813 | −0.00276431 | −0.00306262 | −0.00294361 | 0 | 0 |
| 140491 | Ppp1r3a | 0.001391943 | 0.001546711 | −0.00069041 | −0.00124588 | −0.00035106 | 0 | 0 |
| 246728 | Oas2 | 0.001789663 | −0.00025905 | −0.00067454 | −0.00029751 | 0.000830821 | 0 | 0 |
| 13051 | Cx3cr1 | 0.002171683 | 0.004396701 | 0.006472721 | 0.003738235 | 0.004168838 | 0 | 0 |
| 434130 | Ccdc8 | −0.00188803 | −0.00227065 | −0.00301721 | −0.00372793 | −0.00596372 | 0 | 0 |
| 226049 | Dmrt2 | 0.001785814 | 0.000885306 | 0.003429065 | 0.002854454 | 0.00152348 | 0 | 0 |
| 269116 | Nfasc | 0.001820349 | −0.00133259 | 0.000761398 | 0.001083853 | 0.003378975 | 0 | 0 |
| 66333 | Aqp11 | −0.00200684 | −0.00340805 | −0.00541973 | −0.00475858 | −0.00192065 | 0 | 0 |
| 14051 | Eya4 | 0.001719862 | 0 | 0.000301752 | 0.000571847 | 0.000888956 | 0 | 0 |
| 107272 | Psat1 | 0.001760448 | −0.00135209 | 0.006108646 | 0.004519709 | 0.003050368 | 0 | −0.13792507 |
| 103149 | Upb1 | −0.00123876 | −0.00117561 | 0.002238701 | 0.002123915 | 0.000376673 | 0 | 0 |
| 52187 | Rragd | 0.002248163 | −0.00177436 | 0.000642718 | 0.000802021 | 0.00245718 | 0 | 0 |
| 18227 | Nr4a2 | 0.002530518 | 0.00047649 | 0.002946259 | 0.002997972 | 0.002380095 | 0 | 0 |
| 27425 | Atp5l | −0.00185373 | 0.000214307 | −0.00225812 | −0.00227727 | −0.00052097 | 0 | 0 |
| 12443 | Ccnd1 | −0.00176318 | 0.000316095 | 0.001679529 | −0.00040841 | −0.00053325 | 0 | 0.307726609 |
| 78894 | Aacs | −0.0019844 | −0.00222084 | −0.00323258 | −0.00346868 | −0.00253441 | 0 | 0 |
| 66197 | Cks2 | −0.00216168 | 5.16E−05 | −0.00280334 | −0.00259153 | −0.00368664 | 0 | 0 |
| 14114 | Fbln1 | −0.00170933 | 0.003777449 | −0.00054201 | −0.00152612 | −0.00140107 | 0 | −0.0956303 |
| 76041 | Ccdc125 | −0.00174218 | −0.0017563 | −0.00469126 | −0.00342487 | −0.00336129 | 0 | 0 |
| 21366 | Slc6a6 | 0.002343972 | −0.00032405 | 0.003222526 | 0.003148456 | 0.00285262 | 0 | 0 |
| 71934 | Car13 | 0.002256332 | 6.39E−05 | 0.003010006 | 0.003839832 | 0.003571123 | 0 | 0 |
| 20365 | Serf1 | −0.00223494 | 0.001302524 | −0.00099937 | −0.00195558 | −0.00149832 | 0 | 0 |
| 219132 | Phf11d | 0.001926913 | −9.25E−05 | 0.004150169 | 0.003455492 | 0.003644356 | 0 | −0.17668406 |
| 20541 | Slc8a1 | −0.00184866 | −0.00172896 | −0.00392086 | −0.00188248 | −0.00081817 | 0 | 0.09849254 |
| 17388 | Mmp15 | −0.00131719 | 0.001143303 | −0.00237569 | −0.00168057 | 0.000622142 | 0 | 0 |
| 13131 | Dab1 | 0.001984973 | 0.001006796 | 0.002571079 | 0.001177882 | 0.000948716 | 0 | 0 |
| 54409 | Ramp2 | −0.0019483 | 0.000866921 | −0.00240117 | −0.00140103 | −0.00267014 | 0 | 0 |
| 60533 | Cd274 | 0.001323662 | −0.00108111 | −0.0026811 | −0.00575899 | −0.00273478 | 0 | 0 |
| 56312 | Nupr1 | 0.001810612 | 0.001842135 | 0.004687739 | 0.006248543 | 0.0045989 | 0 | −0.11839156 |
| 53896 | Slc7a10 | −0.00223913 | 0.00059752 | −0.00328593 | −0.00102835 | −0.0023769 | 0 | 0 |
| 16777 | Lamb1 | −0.00187358 | −0.00070847 | −0.0049152 | −0.00500698 | −0.00326113 | 0 | 0 |
| 268930 | Pkmyt1 | −0.00208374 | −0.00168975 | −0.00257554 | −0.00528285 | −0.00091331 | 0 | 0 |
| 71664 | Mettl7b | −0.00191256 | 0.001826853 | −0.00149329 | −0.002844 | −0.00218403 | 0 | 0 |
| 12501 | Cd3e | 0.001846868 | 0.001318817 | 0.000237953 | 0.002468304 | 0.000863201 | 0 | 0.147719556 |
| 77015 | Mpped2 | −0.00126252 | −0.00218829 | −0.00236198 | −0.00212504 | −0.00102833 | 0 | 0.115405169 |
| 407800 | Ecm2 | −0.00204989 | −0.0020182 | −0.00434032 | −0.00594284 | −0.00288804 | 0 | 0 |
| 239273 | Abcc4 | 0.001735869 | 0.000979343 | −0.00072205 | −0.0014955 | −0.0003925 | 0 | 0 |
| 54486 | Hpgds | 0.002301426 | 0.002330655 | 0.00214959 | 0.00129982 | 0.004550799 | 0 | 0 |
| 13180 | Pcbd1 | 0.002474314 | 0.001084585 | 0.001212501 | −0.00129457 | 0.001606983 | 0 | 0 |
| 14544 | Gda | 0.001786304 | 0.000597165 | 0.000230643 | −0.00017217 | 0.001139932 | 0 | 0 |
| 78751 | Zc3h6 | 0.001967528 | −0.00073663 | 0.001495754 | 0.001874102 | 0.003885148 | 0 | 0 |
| 50909 | C1ra | 0.002192249 | 0.000891241 | 0.002944961 | 0.00102436 | 0.001378374 | 0 | 0 |
| 30951 | Chx8 | −0.00206742 | −0.00100967 | −0.00200709 | −0.00127885 | −0.00227937 | 0 | 0 |
| 68075 | Lurap1 | 0.001652825 | 0 | 0.000712702 | 0.001245369 | 0.001762898 | 0 | 0 |
| 72713 | Angptl1 | −0.00248794 | −0.00037002 | −0.00577764 | −0.0049732 | −0.00444598 | 0 | 0 |
| 53601 | Pcdh12 | −0.00180669 | 0.002633565 | 0.000693125 | 0.002490611 | 0.001428754 | 0 | 0 |
| 14254 | Flt1 | 0.001649106 | 0.001459934 | 0.004268078 | 0.003965724 | 0.001706411 | 0 | 0 |
| 74470 | Cep72 | −0.00170702 | 0.000313588 | 0.000775581 | 0.003956415 | 9.20E−05 | 0 | 0 |
| 69993 | Chn2 | −0.0019665 | −0.00097462 | −0.00265328 | −0.00409107 | −0.00286345 | 0 | 0 |
| 171531 | Mlph | 0.002088362 | 0.001706461 | 0.001419552 | 0.004060588 | 0.001634438 | 0 | 0 |
| 108114 | Slc22a7 | −0.00112224 | −0.00141349 | −0.00084722 | −0.00021903 | −0.00233839 | 0 | 0.07558015 |
| 12428 | Ccna2 | −0.00196738 | −0.00458823 | −0.00463519 | −0.00587548 | −0.00242635 | 0 | 0 |
| 67383 | Carnmt1 | −0.00228825 | 6.42E−05 | −0.00281458 | −0.00301279 | −0.00171854 | 0 | 0 |
| 26559 | Hunk | −0.00193845 | −0.00067643 | −5.86E−05 | −0.00032276 | −0.00090972 | 0 | 0.102214179 |
| 448987 | Fbxl7 | −0.00233595 | 0 | −0.00316689 | −0.00524389 | −0.00376145 | 0 | 0 |
| 71819 | Klf23 | −0.00244713 | −0.00034652 | −0.0009447 | −0.00018706 | −0.00177781 | 0 | 0.002617904 |
| 60613 | Kcnq4 | −0.00182959 | 0.000835312 | −0.00011817 | −0.00061839 | −0.00011245 | 0 | 0 |
| 17389 | Mmp16 | −0.00154678 | −0.00037747 | −0.00093524 | −0.00204931 | −0.00146468 | 0 | 0 |
| 16409 | Itgam | 0.001342241 | 0.000920425 | 0.004437098 | 0.004709354 | 0.002310152 | 0 | 0.02826685 |
| 14700 | Gng10 | −0.00216898 | −7.85E−05 | −0.00636386 | −0.00698563 | −0.0036275 | 0 | 0 |
| 241694 | Ralgapa2 | 0.001850191 | −0.00080105 | 0.003202442 | 0.002178331 | 0.002114767 | 0 | 0 |
| 20250 | Scd2 | −0.00147802 | −0.0022007 | −0.00264561 | −0.00183048 | −0.00079276 | 0 | 0.237502315 |
| 50528 | Tmprss2 | 0.001962817 | 0.0017472 | 0.003302471 | 0.003437133 | 0.002816819 | 0 | −0.00190886 |
| 380921 | Dgkh | −0.00170289 | −0.00321995 | −0.00148086 | −0.00170566 | −0.00115993 | 0 | 0 |
| 16874 | Lhx6 | −0.00186743 | 0.002892765 | −0.00171088 | −0.00098512 | −0.00170227 | 0 | 0 |
| 66929 | Asf1b | 0.00170223 | 0.000195722 | 0.004828165 | 0.002970377 | 0.001829803 | 0 | 0 |
| 15463 | Agfg1 | −0.00215893 | −0.00187374 | −0.00423362 | −0.00375104 | −0.00246663 | 0 | 0 |
| 277978 | Exoc3l | −0.00190694 | 0.001455801 | −0.00067043 | −0.00109543 | −0.00078759 | 0 | 0 |
| 52829 | Lurap1l | −0.00163548 | 7.09E−05 | 0.002407544 | 0.002658237 | 0.000573835 | 0 | 0 |
| 214951 | Rhbdl1 | −0.00234653 | 0.002801904 | −0.00092983 | −0.00353469 | −0.00126923 | 0 | 0 |
| 16399 | Itga2b | 0.00208945 | −0.0012632 | 0.001328857 | 0.001715532 | 0.001862199 | 0 | 0 |
| 66094 | Lsm7 | −0.00146252 | 0.001681436 | 0.000776754 | 0.001579126 | 0.000210368 | 0 | 0 |
| 73173 | Pcdh18 | −0.0016199 | 0.000181963 | −0.00155498 | −0.00049438 | 0.000252596 | 0 | 0 |
| 229474 | Fhdc1 | 0.001710744 | 2.72E−05 | −0.00047169 | −0.00185269 | 0.000170075 | 0 | 0 |
| 432530 | Adcy1 | 0.00169326 | 0.002397386 | −0.00014732 | −0.00169903 | 0.00177917 | 0 | 0 |
| 13076 | Cyp1a1 | −0.00195013 | 0.001568691 | −0.00274585 | −0.00225785 | −0.00134557 | 0 | 0 |
| 67674 | Trmt112 | −0.00185318 | −0.00132862 | −0.00391005 | −0.00274443 | −0.00108104 | 0 | 0 |
| 11684 | Alox12 | 0.001642264 | −0.00282587 | 0.000675401 | 0.000272956 | 0.002225526 | 0 | 0 |
| 74393 | Map10 | −0.00231087 | −0.00210668 | −0.0057775 | −0.00555378 | −0.00222477 | 0 | 0 |
| 56532 | Ripk3 | −0.00145147 | −0.00142835 | −0.00301212 | −0.0023819 | −0.0026756 | 0 | 0 |
| 243910 | Nfkbid | −0.00196899 | −0.00169848 | −0.00167186 | −0.00364959 | −0.00155033 | 0 | 0 |
| 11807 | Apoa2 | −0.00033279 | 0.001032919 | −0.00208501 | −0.00365994 | −0.00464026 | 0 | 0 |
| 26381 | Esrrg | 0.001702223 | 0.000889965 | 0.001906828 | 0.00107897 | 0.001531321 | 0 | 0 |
| 80861 | Dhx58 | 0.00213588 | 0.004371768 | 0.004297444 | 0.00390658 | 0.003863677 | 0 | 0 |
| 269831 | Tspan12 | −0.0015546 | 0.001049226 | 0.000716489 | 7.07E−05 | −0.00088343 | 0 | 0 |
| 319387 | Adgrl3 | −0.00180575 | −0.00216728 | −0.00270615 | −0.00079441 | −0.00099441 | 0 | 0 |
| 70445 | Cd248 | −0.00150877 | −0.00041052 | −0.00233829 | −0.00138588 | −0.00309388 | 0 | 0.008564525 |
| 12953 | Cry2 | 0.001868836 | −6.61E−05 | 0.000654401 | 0.001497473 | 0.00114002 | 0 | 0 |
| 118449 | Synpo2 | 0.002104035 | −0.0032612 | −0.00147647 | −0.00284468 | 0.000607262 | 0 | 0.036675119 |
| 320844 | Amigo3 | −0.00126894 | 0.000218873 | −0.00020488 | −0.0008395 | −0.00110215 | 0 | 0 |
| 330189 | Tmem120b | −0.001957 | −0.00372934 | −0.00481259 | −0.00530196 | −0.00237268 | 0 | 0 |
| 18552 | Pcsk5 | −0.0019346 | −0.00070109 | −0.00146197 | −0.0011946 | −0.00199867 | 0 | 0 |
| 71653 | Shtn1 | 0.002229362 | −9.67E−05 | 0.001673206 | 0.00162075 | 0.00140887 | 0 | −0.06962035 |
| 69590 | Gpx8 | −0.00192134 | −0.00245745 | −0.00268319 | −0.00353655 | −0.00219338 | 0 | 0 |
| 21857 | Timp1 | 0.00204009 | 0.000953375 | 0.001388273 | 0.00078935 | −0.00014 | 0 | 0 |
| 20517 | Slc22a1 | 0.000547101 | 0.001386429 | 0.004252035 | 0.002848656 | 0.001103147 | 0 | 0 |
| 14211 | Smc2 | −0.00147741 | −0.00149946 | −0.00402056 | −0.00200974 | −0.00361292 | 0 | 0 |
| 242341 | Atp6v0d2 | 0.001787577 | 0.003447688 | 0.0001726 | 0.000673856 | 0.001542833 | 0 | 0 |
| 12817 | Col13a1 | −0.00214193 | −0.00161392 | −0.00381712 | −0.00587039 | −0.00303111 | 0 | 0 |
| 20356 | Sema5a | −0.00239787 | 0.000227325 | −0.00367621 | −0.00329502 | −0.00226142 | 0 | 0 |
| 11568 | Aebp1 | 0.001786233 | 0.004165535 | 0.001635226 | 0.004026383 | 0.003290509 | 0 | 0 |
| 11496 | Adam22 | 0.001272462 | −0.00272024 | 0.001690103 | 0.003117249 | 0.00120984 | 0 | 0.025851139 |
| 24055 | Sh3bp2 | −0.00119065 | 0.000255734 | −0.00109562 | −0.0013731 | −0.00124646 | 0 | 0 |
| 140483 | Hnmt | 0.001053237 | 0.001902747 | 0.003060959 | 0.000449707 | 0.0014095 | 0 | 0 |
| 14799 | Gria1 | −0.00179984 | −0.00033441 | −0.00016498 | −0.0003583 | −0.00078248 | 0 | 0 |
| 268373 | Ppla | −0.00128767 | −0.00041967 | −0.00081431 | −0.00106891 | −0.00104637 | 0 | 0 |
| 66421 | 2410004B18Rik | −0.00180124 | 0.001196655 | −0.00108126 | −0.00079665 | −0.00185409 | 0 | 0 |
| 56711 | Plag1 | 0.001453687 | 0.001771819 | 0.003306046 | 0.001924431 | 0.005800181 | 0 | −0.12145677 |
| 73368 | Col20a1 | −0.00157889 | −0.00064504 | −0.00245071 | −0.00312134 | −0.004794 | 0 | 0.004024416 |
| 244152 | Tsku | −0.00150511 | −0.00449816 | −0.00161782 | −0.00278365 | −0.00040055 | 0 | 0.199726492 |
| 64008 | Aqp9 | 0.001617526 | 0.003230414 | 0.003015988 | 0.003168084 | 0.000239775 | 0 | 0 |
| 21752 | Tert | −0.00147002 | −0.001142 | −0.00335423 | −0.00284198 | −0.00197347 | 0 | 0 |
| 13837 | Epha3 | −0.00220494 | −0.00143565 | −0.00459571 | −0.00378042 | −0.00303734 | 0 | 0 |
| 78388 | Mvp | 0.002105108 | 0.001343821 | 0.002654297 | 0.003213185 | 0.0029043 | 0 | 0 |
| 15016 | H2-Q5 | 0.002580986 | −0.00027586 | 0.00227001 | 0.001982113 | 0.003047342 | 0 | 0 |
| 104174 | Gldc | 0.001877239 | 0.00202847 | 0.001815277 | 0.000765071 | −0.00011051 | 0 | 0 |
| 13557 | E2f3 | −0.00160131 | −0.00164228 | −0.00276763 | −0.00358616 | −0.00364751 | 0 | 0 |
| 20090 | Rps29 | −0.00164288 | 0.000793218 | −0.00161632 | −0.00175376 | −0.00155963 | 0 | 0 |
| 319504 | Nrcam | −0.00060489 | 0.003199828 | −0.00127877 | −0.00034227 | 1.85E−05 | 0 | 0 |
| 399558 | Flrt2 | −0.00113662 | −0.00093031 | −0.00262555 | −0.00292258 | 0.000823494 | 0 | 0.094273904 |
| 241556 | Tspan18 | −0.00138106 | 0.001997927 | 0.001035734 | 0.002015244 | 0.000902911 | 0 | −0.00170293 |
| 246730 | Oas1a | 0.001226946 | 0.000880398 | 0.001311491 | 0.00084052 | 0.001515941 | 0 | 0 |
| 19013 | Ppara | 0.001072747 | 0.00015911 | 0.003282156 | 0.000153563 | 0.000276952 | 0 | 0 |
| 74318 | Hopx | −0.00123114 | 0.001533961 | −0.00271956 | −0.0005169 | −3.01E−06 | 0 | 0 |
| 268890 | Lsamp | −0.00137346 | 0 | −0.00258022 | −0.00323582 | 0.000645051 | 0 | 0 |
| 106861 | Abhd3 | 0.001323368 | −0.00060296 | 0.003276619 | 0.001797481 | 0.001412505 | 0 | 0 |
| 22271 | Upp1 | 0.002585457 | −0.00171659 | 0.004833064 | 0.005558331 | 0.005784848 | 0 | 0 |
| 26459 | Slc27a5 | −0.00246924 | 0.002109056 | 0.002508967 | 0.003211508 | 0.001263265 | 0 | 0 |
| 53422 | Ybx2 | −0.0024311 | −0.00257134 | −0.00400537 | −0.00551629 | −0.00338169 | 0 | 0.014867238 |
| 381413 | Gpr176 | 0.002418154 | 0 | 0.00278043 | 0.002727896 | 0.001765312 | 0 | 0 |
| 19122 | Prnp | 0.002416766 | 0.002818485 | 0.004428649 | 0.003673887 | 0.003696911 | 0 | 0 |
| 19264 | Ptprc | 0.002405559 | 8.37E−05 | 0.003825778 | 0.004473743 | 0.003283311 | 0 | 0 |
| 84682 | Cox4l2 | −0.00235243 | 0 | −0.00345212 | −0.00197205 | −0.00159101 | 0 | 0 |
| 109828 | C7 | −0.00234706 | 0 | −0.00321441 | −0.00531541 | −0.00520954 | 0 | 0 |
| 320557 | Fam169a | 0.002336762 | 0 | 0.000356603 | 0.001504659 | 0.001019292 | 0 | 0 |
| 14125 | Fcer1a | 0.00233357 | −8.36E−05 | 0.00365979 | 0.004144029 | 0.003799108 | 0 | 0 |
| 109731 | Maob | 0.002314902 | 0.002323551 | 0.003716315 | 0.004278542 | 0.001741863 | 0 | 0 |
| 72119 | Tpx2 | −0.00231424 | 0.000426818 | 0.00096676 | 0.002840395 | 0.000461507 | 0 | 0 |
| 65221 | Slc15a3 | 0.00230242 | −0.00058715 | 0.003091677 | 0.001835049 | 0.002599904 | 0 | −0.02452113 |
| 66060 | Cystm1 | 0.002252752 | −4.43E−05 | 0.000540317 | 0.002977894 | 0.002391103 | 0 | 0 |
| 52588 | Tspan14 | −0.00224504 | −0.00044572 | −0.00450046 | −0.00630048 | −0.00583249 | 0 | 0 |
| 66938 | Sh3d21 | −0.00224154 | −0.00110543 | −0.003812 | −0.00374129 | −0.00283374 | 0 | 0 |
| 69809 | 1810046K07Rik | 0.002229871 | 0.002672616 | 0.008777769 | 0.007581654 | 0.005530698 | 0 | 0 |
| 14163 | Fgd1 | −0.00222143 | −0.00093118 | −0.00333659 | −0.00481511 | −0.00407341 | 0 | 0 |
| 218763 | Lrrc3b | −0.00221479 | −0.0010301 | −0.00313855 | −0.00450173 | −0.0067997 | 0 | 0 |
| 227627 | Obp2a | 0.002207914 | 0.000911078 | 0.003934193 | 0.003735313 | 0 | 0 | 0 |
| 50765 | Tfr2 | 0.002189102 | 0.003314153 | 0.004123854 | 0.005076052 | 0.005133165 | 0 | 0 |
| 12269 | C4bp | 0.002183388 | 0.004519382 | −0.0011383 | 0.002268441 | 0.002006123 | 0 | 0 |
| 66771 | Gld4 | −0.00217928 | −0.00214027 | −0.00350677 | −0.00255545 | −0.00280091 | 0 | 0 |
| 235527 | Plscr4 | 0.002173487 | 0.003027504 | 0.003730256 | 0.004418062 | 0.003504785 | 0 | 0 |
| 70747 | Tspan2 | −0.00217143 | −0.00087292 | −0.00204191 | −0.00266649 | −0.00173607 | 0 | 0 |
| 17314 | Mgmt | 0.002162854 | 0.004524043 | 0.001631792 | 0.002764693 | 0.003062001 | 0 | 0 |
| 20620 | Plk2 | 0.002153245 | 0.003033492 | 0.001994412 | 0.0029637 | 0.000105925 | 0 | 0 |
| 16818 | Lck | 0.002151417 | 0.002179599 | −0.00166813 | 0.000647104 | 0.002178001 | 0 | 0 |
| 13114 | Cyp3a16 | 0 | 0.009832714 | 0.001136108 | 0 | 0 | 0 | −0.00214462 |
| 71753 | Tmprss6 | −0.00084476 | −0.0085273 | −0.00185993 | −0.00045001 | −0.00058362 | 0 | 0 |
| 208677 | Creb3l3 | −0.00012074 | 0.008489042 | 0.004395291 | 0.006013308 | 0.004455387 | 0 | 0 |
| 384783 | Irs2 | 0.000764318 | 0.006904805 | 0.004521857 | 0.003795976 | 0.005298065 | 0 | −0.0170761 |
| 239463 | Fam83a | 0 | −0.00680657 | −0.00251504 | 0 | −0.00207101 | 0 | 0.119583383 |
| 110033 | Kif22 | 0.000287936 | 0.00677205 | 0.002638438 | 0.003837759 | 0.000977108 | 0 | 0 |
| 17842 | Mup3 | −0.00069371 | 0.006565083 | −0.0022536 | −0.00221237 | 0 | 0 | 0 |
| 22287 | Scgb1a1 | −0.00044043 | 0.006548163 | 0.002934619 | 0.002529977 | 0.000659513 | 0 | 0 |
| 68732 | Carmil1 | −0.00031114 | −0.00641682 | −0.00339981 | −0.00054642 | −0.00196897 | 0 | 0 |
| 100039008 | Mup10 | 0 | 0.006405192 | 0 | 0 | 0 | 0 | −0.37197072 |
| 18636 | Cfp | −0.00031222 | 0.006371872 | −0.00088891 | −0.00049486 | −0.00013955 | 0 | 0 |
| 16523 | Kcnj8 | −0.00127962 | −0.00635385 | −0.00493445 | −0.00522045 | −0.00170301 | 0 | 0 |
| 30839 | Fbxw5 | 0.001022055 | 0.006317279 | 0.004655115 | 0.005233556 | 0.007939573 | 0 | 0 |
| 75974 | Dock11 | 0.000350428 | 0.006302881 | 0.001116264 | 0.002849762 | 0.000175615 | 0 | 0 |
| 56708 | Clcf1 | −0.00031138 | −0.00629961 | −0.00693249 | −0.00781577 | −0.00329738 | 0 | 0.234870038 |
| 103988 | Gck | −0.00137981 | −0.00627478 | −0.00716765 | −0.00707963 | −0.00559129 | 0 | 0 |
| 71982 | Snx10 | 0.000983485 | −0.00625106 | −0.0003043 | −0.00092305 | 8.21E−05 | 0 | 0 |
| 102626 | Mapkapk3 | 0.000754128 | −0.00622973 | −0.00286375 | −0.00283144 | −0.00078522 | 0 | 0.031837129 |
| 15006 | H2-Q1 | 0 | 0.006225719 | 0 | 0 | 0 | 0 | −0.13251346 |
| 13419 | Dnase1 | 0.0002602 | 0.006221771 | 0.003238704 | −0.00152788 | −0.00085132 | 0 | 0 |
| 230784 | Sesn2 | 0.001502583 | 0.006157914 | 0.003965075 | 0.00521056 | 0.004333237 | 0 | −0.01096644 |
| 68371 | Pbld1 | −0.00061494 | 0.006082976 | 0.003958479 | 0.00270896 | 0 | 0 | −0.69535335 |
| 546336 | Prrg1 | 0.001185755 | 0.0059721 | 0.003323297 | 0.004011769 | 0.001419807 | 0 | −0.10956581 |
| 11425 | Apoc4 | 0.001081512 | 0.005859451 | 0.003040526 | 0.002782982 | 0.002045391 | 0 | 0 |
| 16364 | Irf4 | −0.00029606 | 0.005822451 | 0.004691345 | 0.006505457 | 0.001826394 | 0 | −0.11974817 |
| 320292 | Rasgef1b | 0.000435546 | 0.005818796 | 0.003896567 | 0.002209781 | 0.000827286 | 0 | −0.22092883 |
| 337924 | Cyp3a44 | 0 | −0.00575848 | 0 | 0 | 0 | 0 | 0.108731857 |
| 229722 | Blapor1 | −0.00017074 | −0.00566793 | −0.00242979 | −0.00352273 | −0.0003397 | 0 | 0.003681109 |
| 17843 | Mup4 | 0 | 0.005637803 | 0 | 0 | 0 | 0 | −0.52662764 |
| 23960 | Qas1g | 0 | 0.005618699 | 0.002996163 | 0 | 0 | 0 | −0.1284371 |
| 68195 | Rnaset2b | 0.000335808 | 0.005605547 | 0.002758549 | 0.000980898 | 0 | 0 | 0 |
| 240186 | Zfp438 | 0.001347767 | 0.005546124 | 0.005121575 | 0.004385394 | 0.00098514 | 0 | 0 |
| 56386 | B4galt6 | −0.0001394 | 0.00554241 | 0.001161688 | 0.000895046 | 9.44E−05 | 0 | 0 |
| 70261 | Chp2 | −0.00061619 | −0.00551823 | −0.00303003 | −0.00710904 | −0.00360791 | 0 | 0 |
| 16469 | Jrk | −0.00022859 | −0.00549737 | −0.00477489 | −0.00556662 | −0.00228218 | 0 | 0 |
| 22445 | Xlr3a | 0 | 0.005490582 | 0.002812833 | 0 | 0 | 0 | 0 |
| 320924 | Ccbe1 | 0.000455507 | 0.005463384 | 0.003402367 | 0.0022169 | 0.001142869 | 0 | 0 |
| 70123 | Nbdy | 0.000420425 | 0.005450715 | 0.005592301 | 0.00556137 | 0.002896283 | 0 | 0 |
| 13118 | Cyp4a12b | 0 | −0.00544845 | 0 | 0 | 0 | 0 | 1.189086251 |
| 13098 | Cyp2c39 | −0.00143164 | 0.00544515 | 0.003280247 | 0.00418083 | 0 | 0 | −0.03189093 |
| 17395 | Mmp9 | 0.000542173 | 0.005408302 | −0.00186883 | 0.002359699 | −0.0001772 | 0 | 0 |
| 69454 | Clic3 | −0.00044403 | −0.00539089 | −0.00215155 | −0.00289132 | −0.00189712 | 0 | 0.019617665 |
| 100705 | Acacb | 0.001345073 | −0.00536002 | −0.0012538 | −0.00069998 | 0.001238988 | 0 | 0.047376708 |
| 17069 | Ly6e | 0.001130919 | 0.005352166 | 0.001314017 | 0.000240837 | −0.00013551 | 0 | 0 |
| 380839 | Serpinb1c | 0 | −0.00532891 | 0 | 0 | 0 | 0 | 0.009218004 |
| 74365 | Lonrf3 | 0.000732784 | 0.005310428 | −0.00047016 | 0.001745411 | 0.00236654 | 0 | 0 |
| 16765 | Stmn1 | −0.00042343 | 0.005300557 | 0.006051347 | 0.006336945 | 0.001653468 | 0 | 0 |
| 381218 | Spata6l | 0.000738969 | 0.005260581 | 0.004187246 | 0.004890635 | 0.003730379 | 0 | −0.21199495 |
| 71780 | Isyna1 | −0.00042596 | 0.005218512 | 0.000823871 | 0.000419023 | −0.00108775 | 0 | 0.321419268 |
| 27981 | Rsrp1 | −0.00133536 | −0.00520613 | −0.00568172 | −0.00610378 | −0.00381904 | 0 | 0.010314513 |
| 71755 | Dhdh | −0.00011146 | −0.00518017 | −0.00469934 | −0.00457677 | −0.00270301 | 0 | 0 |
| 14289 | Fpr2 | 0 | −0.00514854 | −0.00501376 | 0 | 0 | 0 | 0 |
| 66569 | Gdpd1 | 0.000660901 | −0.00513852 | −0.00041761 | −0.00119671 | −0.00060913 | 0 | 0 |
| 72082 | Cyp2c55 | −0.00024608 | −0.00512369 | −0.00335964 | −0.00388401 | −0.00442555 | 0 | 0 |
| 72361 | Ces2g | −0.00156931 | −0.00510851 | −0.00616307 | −0.00745097 | −0.00461313 | 0 | 0.208793357 |
| 16145 | Igtp | 0.000554254 | 0.005104931 | −0.00129413 | −0.00059709 | 0.001082148 | 0 | −0.00532076 |
| 59608 | Sec24d | −0.00090522 | −0.0050831 | −0.00243136 | −0.00310228 | −0.00272483 | 0 | 0 |
| 107448 | Unc5a | 0.000933899 | 0.005073742 | 0.001669613 | 0.00085523 | 0.003850731 | 0 | 0 |
| 66253 | Aig1 | 0.00044886 | −0.00507102 | −0.00293833 | −0.00308064 | −0.00161332 | 0 | 0 |
| 330406 | B4galnt3 | −0.00034743 | −0.00506355 | −0.00382584 | −0.00167541 | 0.000350307 | 0 | 0 |
| 269346 | Slc28a2 | −0.0004273 | 0.00506183 | 0.001295559 | 0.00116002 | 0.000523577 | 0 | 0 |
| 628813 | Gm11437 | 0.000247293 | −0.00504188 | −0.00398729 | −0.00253628 | −0.00077383 | 0 | 0.008658362 |
| 13730 | Emp1 | 0.000369779 | −0.00501752 | −0.00097179 | −0.00025831 | 0.000182716 | 0 | 0 |
| 13094 | Cyp2b9 | 0 | 0.005005606 | 0.005999948 | 0 | 0 | 0 | −0.6045708 |
| 16326 | Inhbe | 0.000580111 | −0.00499483 | −0.00201682 | −0.00260964 | −0.0009729 | 0 | 0.08519172 |
| 12583 | Cdo1 | −1.20E−05 | −0.00498509 | −0.00092702 | −0.00276382 | −0.00118585 | 0 | 0 |
| 18588 | Pde6g | 0.000347902 | 0.004981498 | 0.001395389 | 0.001208185 | 0.000745401 | 0 | 0 |
| 231821 | Adap1 | −0.00094889 | 0.004977779 | 0.000251879 | −2.77E−05 | −0.00158821 | 0 | 0 |
| 101831 | Faap24 | −0.00057125 | 0.004975493 | 0.00145552 | 0.001931091 | 0.001431054 | 0 | 0 |
| 207259 | Zbtb7c | −0.00015707 | 0.004963642 | 0.002191672 | 0.002701956 | −0.00021195 | 0 | 0 |
| 71699 | Slc41a3 | −0.00092361 | 0.004955218 | 0.0012173 | −0.00011332 | −0.00155149 | 0 | 0 |
| 74694 | Tbc1d30 | 0.000296074 | 0.004946948 | 0.004325011 | 0.003784497 | 0.000215965 | 0 | 0 |
| 18828 | Plscr2 | 0.001677843 | 0.004944914 | 0.006591553 | 0.005220724 | 0.004386865 | 0 | 0 |
| 55963 | Slc1a4 | −0.00024411 | −0.00493374 | 0.001065595 | 0.001028084 | 0.003011785 | 0 | −0.01144602 |
| 70370 | Fbln7 | 0.001608837 | 0.004900084 | 0.00056459 | 0.003396796 | 0.00184976 | 0 | −0.04664903 |
| 217517 | Stxbp6 | 0.000190101 | 0.004895138 | 0.002124024 | 0.001034721 | −0.00070617 | 0 | 0 |
| 16325 | Inhbc | 0.000118585 | −0.00489303 | 0.000532514 | 0.00137896 | 0.00219417 | 0 | 0.009337762 |
| 56741 | Igdcc4 | 0.000284403 | 0.004880761 | 0.001782702 | 0.005267429 | 0.001566605 | 0 | 0 |
| 245050 | Gask1a | −0.00034524 | −0.00487738 | −0.00274938 | −0.00085367 | −0.0004642 | 0 | 0.012967669 |
| 11944 | Atp4a | −0.00021354 | −0.00487575 | −0.00120246 | −0.00047515 | 0.000740423 | 0 | 0 |
| 242316 | Gdf6 | 0 | 0.004872695 | 0 | 0 | 0.001975339 | 0 | 0 |
| 98660 | Atp1a2 | −0.00072101 | −0.0048524 | −0.00610552 | −0.00568172 | −0.0037647 | 0 | 0.033178073 |
| 18242 | Oat | −2.15E−05 | −0.00484331 | −0.00249244 | −0.00334237 | −0.00237353 | 0 | 0 |
| 213019 | Pdlim2 | −0.00081013 | −0.00484085 | −0.00105786 | 0.0006911 | 0.001285951 | 0 | 0 |
| 215335 | Slc36a1 | −0.00107491 | 0.004840508 | 0.003331642 | 0.004070586 | 0.0013788 | 0 | −0.01530998 |
| 17035 | Lxn | −0.00084989 | 0.004833166 | 0.00068379 | 4.88E−05 | −0.00189776 | 0 | 0 |
| 668212 | Efr3b | −0.0002829 | 0.004811671 | 0.002763438 | 0.000972553 | 0.0001294 | 0 | 0 |
| 15186 | Hdc | 0.000814966 | 0.00480268 | 0.004366373 | 0.007510204 | 0.00518217 | 0 | −0.01420787 |
| 66506 | Psmg3 | 0.000269563 | 0.004798556 | 0.003312081 | 0.004173916 | 0.001603419 | 0 | 0 |
| 230903 | Fbxo44 | 0.000251824 | −0.00479544 | −0.00461666 | −0.00449153 | −0.00200668 | 0 | 0 |
| 71693 | Colec11 | 0.000106774 | −0.00479152 | 0.000567474 | −0.0015989 | 0.0013023 | 0 | 0 |
| 382053 | Ces3a | 0 | 0.004784648 | 0 | 0 | 0 | 0 | 0 |
| 21822 | Tgtp1 | −0.00077282 | 0.004756605 | −0.00060926 | −0.001608 | 0 | 0 | −0.04268334 |
| 22029 | Traf1 | −0.00103054 | −0.00475579 | −0.00258322 | −0.00438653 | −0.00284364 | 0 | 0.006801646 |
| 381531 | Mup21 | 0 | 0.004752852 | 0 | 0 | 0 | 0 | 0 |
| 665563 | Mthfd2l | 0.000226579 | 0.004744113 | 0.002966407 | 0.00294593 | 0.004656153 | 0 | 0 |
| 232934 | Mypop | 0.000606622 | 0.004741259 | −0.00052242 | 0.001631198 | 0.002591213 | 0 | 0 |
| 77864 | Ypel2 | 0.000828442 | 0.004739862 | 0.002487588 | 0.002491367 | 0.002506672 | 0 | −0.09956632 |
| 171281 | Acot3 | −0.00105665 | −0.00473374 | −0.00361706 | −0.00058818 | −0.00145717 | 0 | 0 |
| 30843 | Fbxl12 | 0.000748251 | 0.004717551 | 0.001989595 | 0.005000271 | 0.002219248 | 0 | 0 |
| 76074 | Gbp8 | −0.00077266 | −0.00471011 | 0.000304383 | −0.00096211 | 0 | 0 | 0.072107853 |
| 327957 | Scimp | 0.000231994 | 0.004701254 | 0.002768231 | 0.003995913 | 0.002074896 | 0 | 0 |
| 17922 | Myo7b | 0.000560982 | 0.004698043 | −0.00025555 | 0.001783491 | 0.001352799 | 0 | 0 |
| 235330 | Ttc12 | 0.001747074 | 0.004690029 | 0.006525544 | 0.006967592 | 0.004772211 | 0 | 0 |
| 83675 | Bicc1 | −0.00075285 | 0.004680034 | −9.41E−05 | −0.00120493 | −0.00063717 | 0 | −0.01462264 |
| 16855 | Lgals4 | 0.000437295 | −0.00467524 | 0.000775353 | 0.000980161 | 7.97E−05 | 0 | 0 |
| 67378 | Bbs2 | 0.000254193 | −0.00467503 | −0.00043689 | −0.00170563 | −0.00068341 | 0 | 0 |
| 16571 | Kif4 | −0.00197745 | −0.0046601 | −0.00700398 | −0.00826572 | −0.0037054 | 0.132366155 | 0.076012696 |
| 54167 | Icos | −0.00080044 | −0.0046543 | −0.00339937 | −0.00434316 | −0.00345301 | 0 | 0 |
| 56715 | Rabgef1 | 0.000250119 | 0.004650969 | 0.002090255 | 0.000456963 | 0.002069968 | 0 | 0 |
| 13108 | Cyp2g1 | 2.97E−05 | −0.00463789 | 0.002201248 | 0.002679745 | 0 | 0 | 0 |
| 18048 | Klk1b4 | 0.000408432 | 0.004626611 | 3.85E−05 | −0.00052581 | 0 | 0 | 0 |
| 241303 | Fam78a | −0.00142263 | −0.00461862 | 1.21E−05 | −0.00139534 | −0.00118845 | 0 | 0 |
| 14247 | Fli1 | −0.00131067 | −0.00459776 | −0.00465777 | −0.00265924 | −0.00158427 | 0 | 0 |
| 329650 | Med12l | −0.00055637 | −0.00459645 | −0.0036863 | −0.0037866 | −0.00225846 | 0 | 0 |
| 72445 | Prr5l | −6.75E−05 | −0.00458226 | 0.001762218 | 0.000796055 | −0.00110921 | 0 | 0 |
| 26380 | Esrrb | −0.00046451 | −0.00457702 | −0.00432901 | −0.00466867 | −0.00332442 | 0 | 0.172070289 |
| 319582 | Trmt9b | −1.19E−05 | −0.00455755 | −0.00213362 | −0.0032142 | −0.00386317 | 0 | 0 |
| 11481 | Acvr2b | 4.22E−05 | 0.004536476 | 0.002802274 | 0.002288099 | 0.000511599 | 0 | 0 |
| 72289 | Malat1 | −0.00094018 | −0.0045358 | −0.00443458 | −0.00481958 | 0 | 0 | 0 |
| 68172 | Rpl39l | 0 | 0.004527264 | 0 | 0 | 0 | 0 | 0 |
| 12623 | Ces1g | −0.00062717 | −0.00452134 | −0.00348128 | −0.00333941 | 0 | 0 | 0.302268899 |
| 23948 | Mmp17 | −0.00123386 | −0.00450767 | −0.00239286 | −0.00428879 | −0.00438959 | 0 | 0 |
| 330723 | Htra4 | −3.85E−05 | −0.00450756 | −0.00722678 | −0.0073317 | 0 | 0 | 0.0406789 |
| 329910 | Acct11 | −7.07E−05 | −0.00449632 | −0.00773966 | −0.00760381 | −0.00268594 | 0 | 0 |
| 54608 | Abhd2 | 0.000379648 | 0.004479458 | 0.004803524 | 0.003580528 | 0.002446929 | 0 | −0.17543169 |
| 58992 | F12 | 0.000647303 | 0.004461404 | 0.003658754 | 0.001935115 | 0.001733767 | 0 | 0 |
| 235047 | Zfp809 | 0.000144414 | 0.004455062 | 4.07E−05 | −0.00044014 | −0.00044753 | 0 | 0 |
| 24004 | Rai2 | 0.000850401 | −0.00445151 | −0.00529656 | −0.0065622 | −0.00329386 | 0 | 0 |
| 17929 | Myom1 | 0.000380827 | −0.00445092 | −0.0004535 | −0.00055208 | 0.000900421 | 0 | 0.333885312 |
| 70454 | Cenpl | 7.55E−06 | −0.00442977 | −0.00249324 | −0.0025184 | −0.00193722 | 0 | 0 |
| 18302 | Oit3 | −4.14E−05 | −0.00442649 | −0.00085451 | −0.00106776 | −0.00037845 | 0 | 0 |
| 14939 | Gzmb | 0 | 0.004425702 | 0 | 0 | 0 | 0 | −0.01044912 |
| 60507 | Qtrt1 | −0.00072466 | −0.00441896 | −0.00034446 | −0.00056336 | 0.000673633 | 0 | 0 |
| 327959 | Xaf1 | 0.000396214 | −0.00441368 | −0.00167773 | −0.00238848 | −0.00156695 | 0 | 0.141400659 |
| 232933 | Ccdc61 | −0.00080178 | −0.00440695 | −0.0034707 | −0.00157949 | −0.00065059 | 0 | 0 |
| 63953 | Dusp10 | −0.00102228 | −0.00440555 | −0.00350265 | −0.00419458 | −0.00252154 | 0 | 0 |
| 17768 | Mthfd2 | −0.00034691 | 0.004402664 | −0.00436996 | −0.00438324 | −0.00173383 | 0 | 0 |
| 15200 | Hbegf | −0.00107199 | 0.004394125 | 0.003425882 | 0.001445542 | −0.00117622 | 0 | 0 |
| 257635 | Sdsl | 0.001206894 | −0.0043902 | −0.00257295 | −0.00275431 | −0.0002653 | 0 | 0 |
| 272158 | Poln | 0.000172494 | 0.004382241 | −0.00118205 | −0.00376039 | −0.00170844 | 0 | 0 |
| 212439 | AA986860 | 0.000876349 | 0.004359855 | 0.003822751 | 0.004081662 | 0.002152574 | 0 | −0.05627638 |
| 56356 | Gltp | 0.000581797 | −0.00435702 | −0.00129253 | −0.00133234 | −0.00076951 | 0 | 0 |
| 11555 | Adrb2 | −0.00147438 | −0.00434566 | −0.00123478 | −0.00043018 | −0.00065947 | 0 | 0 |
| 16981 | Lrrn3 | 0.00022988 | −0.00434154 | −0.00592238 | −0.00722488 | −0.00410044 | 0 | 0 |
| 246103 | Atxn7 | 0.001003616 | 0.004337659 | −0.00081169 | −0.00054889 | 0.000869801 | 0 | 0 |
| 93700 | Pcdhgb2 | −0.00092474 | −0.00433462 | −0.0045079 | −0.0042493 | −0.00250541 | 0 | 0.225881293 |
| 17709 | COX2 | 0 | −0.00432802 | 0 | 0 | 0 | 0 | 0 |
| 71132 | Cabyr | −5.45E−05 | −0.00432224 | −0.00638778 | −0.00478666 | −0.00415404 | 0 | 0.329134437 |
| 117606 | Boc | −0.00068068 | −0.00432193 | −0.00267319 | −0.00451615 | −0.00376238 | 0 | 0.080366011 |
| 74747 | Ddit4 | 0.000572435 | 0.004318822 | 0.005743547 | 0.005003157 | 0.004974229 | 0 | −0.21201812 |
| 27362 | Dnajb9 | 0.000707064 | 0.004299794 | 0.002570125 | 0.001718165 | 0.002094225 | 0 | 0 |
| 69034 | Nupr1l | 0.000619821 | 0.004297637 | 0.003249342 | 0.003214939 | 0 | 0 | 0 |
| 214547 | She | 0.000387684 | 0.00428576 | 0.004658111 | 0.0058708 | 0.003517414 | 0 | 0 |
| 215653 | Rassf2 | −0.0002746 | −0.00427792 | −0.00298313 | −0.00328093 | −0.00258078 | 0 | 0.014588082 |
| 212391 | Lcor | −0.00032939 | −0.00427284 | −0.00549769 | −0.00442736 | −0.0026718 | 0 | 0 |
| 101543 | Wtip | 0.000418804 | −0.00426941 | −0.0015834 | −0.0017268 | −0.00018005 | 0 | 0 |
| 19700 | Rem1 | −0.00086929 | −0.00426842 | −0.00411613 | −0.00433172 | −0.0010025 | 0 | 0 |
| 240869 | Zbtb37 | −9.60E−05 | −0.00426336 | −0.00381186 | −0.00203522 | −0.00145078 | 0 | 0 |
| 17844 | Mup5 | 0 | 0.00424035 | 0 | 0 | 0 | 0 | −0.44012785 |
| 27214 | Dbf4 | −0.00028682 | 0.004230424 | 0.001540509 | 0.002272172 | 0.002492687 | 0 | −0.07931005 |
| 319924 | Apba1 | −0.00010965 | 0.004222116 | 0.001343915 | 0.001343894 | 0.002391908 | 0 | 0 |
| 217328 | Myo15b | 0 | −0.00422172 | −0.00493662 | 0 | 0 | 0 | 0.001178072 |
| 68201 | Ccdc34 | 0.000481243 | 0.004218467 | 0.002725084 | 0.004621324 | 0.002271201 | 0 | 0 |
| 18119 | Nodal | 0.000859405 | 0.00421672 | 0.003992825 | 0.004352359 | 0.00229199 | 0 | −0.00446327 |
| 12836 | Col7a1 | −0.00017042 | 0.004216372 | 0.00070942 | 0.000917717 | −0.00212039 | 0 | 0 |
| 13436 | Dnmt3b | −0.00084837 | −0.00421602 | −0.00115362 | −0.00357505 | −0.00139044 | 0 | 0.053664651 |
| 209176 | Ido2 | −0.00072855 | −0.00421476 | −0.00439038 | −0.00556063 | 0 | 0 | 0.413707368 |
| 240327 | Gm4951 | 0.001494458 | −0.00421072 | 0.002756669 | 0.002561574 | 0.002530643 | 0 | 0.208871744 |
| 52850 | Sgsm1 | 0.000728531 | −0.00420892 | −0.00292101 | −0.00125398 | 0.00044678 | 0 | 0 |
| 192136 | Sugct | −6.17E−05 | −0.00420012 | −0.0008854 | 6.66E−05 | 0.000773246 | 0 | 0 |
| 236930 | Ercc6l | −8.42E−05 | 0.004190109 | −0.00015073 | −0.0008977 | −0.0021416 | 0 | 0 |
| 78317 | Ccdc88b | −0.00112798 | −0.00418736 | −0.004118 | −0.0026562 | −0.00302521 | 0 | 0 |
| 15248 | Hic1 | −0.0001605 | −0.00418648 | −0.00231707 | −0.00189711 | 0.001169879 | 0 | 0 |
| 13086 | Cyp2a4 | 0 | 0.004182382 | 0 | 0 | 0 | 0 | 0.043035023 |
| 72480 | Tspyl4 | −0.00108548 | −0.00417526 | −0.00370481 | −0.00309351 | −0.0007621 | 0 | −0.10308881 |
| 668303 | Kif26a | −0.0004333 | −0.00416937 | 0.000479466 | −0.00150986 | −0.00087943 | 0 | 0 |
| 217333 | Trim47 | 0.000437828 | −0.0041684 | 0.000748053 | 0.000115209 | 0.000430647 | 0 | 0 |
| 232333 | Slc6a1 | −0.00121289 | −0.00416731 | −0.00192793 | −0.0058958 | −0.00353936 | 0 | 0 |
| 70061 | Sdr9c7 | 0.000494715 | −0.004167 | 0.000605801 | 0.001213957 | 0.001213554 | 0 | 0.149280214 |
| 17718 | ND3 | 0 | −0.0041622 | 0 | 0 | 0 | 0 | 0 |
| 14461 | Gata2 | 0.000580229 | −0.00415598 | −0.00178662 | −0.00286526 | −0.0027038 | 0 | 0 |
| 58220 | Pard6b | 0.00031392 | −0.0041502 | −0.00107331 | 0.000253394 | −0.00050033 | 0 | 0 |
| 140859 | Nek8 | −0.0005213 | −0.00414674 | −0.00095356 | −0.00073376 | −0.00034508 | 0 | 0 |
| 320977 | A330023F24Rik | 0.001475768 | 0.004138235 | 0.003652942 | 0.004271914 | 0 | 0 | 0 |
| 107566 | Art2bp | −0.00014202 | 0.004133914 | 0.000245835 | −0.00036974 | 0.00190926 | 0 | 0 |
| 69535 | Ten1 | 0.000265148 | 0.004133288 | 9.79E−05 | 0.000638247 | 0.001155674 | 0 | 0 |
| 27062 | Cadps | 0.001304647 | −0.004128 | −0.00134191 | −0.00024607 | 0.00259375 | 0 | 0 |
| 14469 | Gbp2 | 0.000499451 | −0.00412184 | −0.00410387 | −0.00344168 | −0.00351545 | 0 | 0 |
| 18710 | Plk3r3 | 0.000778607 | 0.004119119 | 0.004052521 | 0.003308206 | 0.001968507 | 0 | 0.014017545 |
| 22160 | Twist1 | −0.0007594 | −0.00411904 | −0.00428109 | −0.00149209 | −0.00355292 | 0 | 0.193484497 |
| 70024 | Mcm10 | −0.00039386 | 0.004110675 | 0.004807252 | 0.00357484 | 0.002119464 | 0 | −0.30320763 |
| 100504714 | Gm16793 | 0 | 0.004110519 | 0 | 0 | 0 | 0 | −0.0313353 |
| 105837 | Mtbp | 0.000909366 | −0.00410987 | −0.00115561 | −0.00103952 | 0.000328792 | 0 | 0 |
| 245622 | Fam199x | −0.00088251 | −0.00410612 | −0.00193457 | −0.0013311 | −0.00164987 | 0 | 0 |
| 68910 | Zfp467 | 0.001196019 | −0.00410349 | −0.00230183 | −0.00177977 | −0.00131706 | 0 | 0.00084356 |
| 76044 | Ncapg2 | 0.000636964 | 0.004101887 | 0.004916532 | 0.00594292 | 0.003560589 | 0 | −0.02908584 |
| 11761 | Aox1 | 0.001300426 | −0.0040912 | −0.0054122 | −0.00606952 | −0.00383416 | 0 | 0 |
| 353310 | Zfp703 | 0.000972363 | −0.00408794 | 0.000850416 | 0.00138832 | 0.000745084 | 0 | 0 |
| 20908 | Stx3 | 0.000140349 | −0.00408744 | −0.00013116 | −0.00148904 | −0.00017485 | 0 | 0 |
| 71151 | Eri2 | −0.00104342 | 0.004085067 | −0.00287723 | −0.00363611 | −0.00171856 | 0 | 0 |
| 19057 | Ppp3cc | 0.001576411 | 0.004074917 | 0.00404745 | 0.003971271 | 0.003239807 | 0 | 0 |
| 77739 | Adamtsl1 | −0.00054055 | −0.0040749 | −0.00374172 | −0.00432361 | −0.00201049 | 0 | 0 |
| 22702 | Zfp42 | 0 | −0.00407154 | 0 | 0 | −0.00257881 | 0 | 0 |
| 224840 | Treml4 | −0.00059259 | −0.00407149 | −0.00015865 | −0.00405314 | −0.00280619 | 0 | 0 |
| 15040 | H2-T23 | 0.001205079 | 0.004070128 | 0.002271932 | 0.002935654 | 0.002636519 | 0 | −0.14871647 |
| 231070 | Insig1 | 0.00079924 | −0.00405839 | −0.00214017 | −0.00130242 | −0.00292905 | 0 | 0 |
| 16196 | Il7 | 0.001598779 | 0.004061954 | 0.004750372 | 0.005276467 | 0.00266827 | 0 | −0.0074726 |
| 70574 | Cpm | −0.00031781 | 0.00405631 | 0.002447657 | 0.001629707 | 0.002404328 | 0 | −0.14411388 |
| 14913 | Guca1a | −4.47E−05 | 0.004054183 | 0.000268874 | 8.17E−05 | 0 | 0 | −0.09392281 |
| 96935 | Susd4 | −0.00022758 | −0.00405151 | −0.00389961 | −0.00279663 | −0.00237739 | 0 | 0.311219801 |
| 58185 | Rsad2 | 0.001819127 | −0.0040487 | −0.00060912 | 0.000128137 | 0.000193913 | 0 | 0 |
| 67391 | Fundc2 | 0.001363358 | 0.004047757 | 0.005311612 | 0.006147421 | 0.004290731 | 0 | 0 |
| 21665 | Tdg | −0.00048274 | 0.004045507 | 0.003424449 | 0.00311407 | −0.0001794 | 0 | −0.14333218 |
| 17218 | Mcm5 | −0.00170864 | 0.004045309 | −0.00115871 | −0.00263009 | −0.00044893 | 0 | 0 |
| 68628 | Fbxw9 | 0.000485673 | 0.004031806 | 0.006321418 | 0.0051866 | 0.003015278 | 0 | 0 |
| 211986 | Tmem18 | −0.00025291 | −0.00402431 | −0.00200617 | −0.00342466 | −0.00188278 | 0 | 0 |
| 108961 | E2f8 | −0.0007339 | −0.00402425 | −0.00362393 | −0.004914 | −0.00180603 | 0 | 0 |
| 16011 | Igfbp5 | 0.000943753 | 0.004014441 | 0.003554825 | 0.003453852 | 0.002638326 | 0 | 0 |
| 246293 | Klhl8 | 0.000411647 | 0.004013999 | 0.002525019 | 0.003166151 | 0.001524556 | 0 | −0.03164265 |
| 73230 | Bmper | −0.00073145 | −0.00400685 | −0.00368938 | −0.00571093 | −0.0037785 | 0 | 0.189879395 |
| 56434 | Tspan3 | −0.00074636 | −0.00400421 | −0.00273785 | −0.0034703 | −0.00271766 | 0 | 0 |
| 333424 | A4gnt | 0 | 0.00400213 | 0.001923228 | 0 | 0.002663281 | 0 | 0 |
| 11302 | Aatk | −0.00063055 | 0.003996451 | −0.00170091 | −0.00154576 | −0.001273 | 0 | 0 |
| 69376 | Zpbp2 | −0.00036345 | −0.0039943 | 0 | −0.00295034 | −0.00081752 | 0 | 0 |
| 16000 | Igf1 | 0.000389624 | 0.003993101 | 0.004665498 | 0.005239663 | 0.002793642 | −0.25782954 | −0.32976461 |
| 226744 | Cnst | 0.000293714 | −0.00399206 | −0.00127292 | −0.00123095 | −0.00023509 | 0 | 0.102768728 |
| 108686 | Ccdc88a | −0.00100641 | −0.00399179 | −0.00178417 | −0.00291064 | −0.00271107 | 0 | 0.106396276 |
| 28254 | Slco1a6 | 0 | −0.00398724 | 0 | 0 | 0 | 0 | 0 |
| 23888 | Gpc6 | −0.00116626 | −0.00398159 | −0.00485549 | −0.00737434 | −0.00303379 | 0 | 0 |
| 11657 | Alb | 0.000995765 | 0.001335821 | 0.008574334 | 0.00816347 | 0.003862493 | 0 | 0 |
| 236149 | Slc22a26 | −0.00045571 | −0.00222688 | −0.00823169 | −0.00608042 | 0 | 0 | 0 |
| 14357 | Dtx1 | −0.00170358 | −0.00256725 | −0.00796405 | −0.00767503 | −0.00501857 | 0 | 0 |
| 23890 | Gpr34 | 0.001146857 | 0.001277194 | 0.007777104 | 0.00593034 | 0.003313773 | 0 | 0 |
| 320139 | Ptpn7 | −0.00065023 | 0 | −0.00770619 | −0.0082864 | −0.00528223 | 0 | 0 |
| 19660 | Rbp2 | 0 | −0.00302305 | −0.00750166 | 0 | −0.00694648 | 0 | 0 |
| 57905 | Isy1 | −0.00049078 | −0.00225748 | −0.00733365 | −0.00606166 | −0.00473116 | 0 | 0 |
| 210321 | BC048679 | 0 | 0.000593022 | 0.007174549 | 0 | 0 | 0 | 0 |
| 320712 | Abi3bp | −0.00189131 | −0.00232721 | −0.00717285 | −0.00636185 | −0.00373135 | 0 | 0 |
| 83674 | Cnnm1 | 0.002063734 | 0.000935139 | 0.007117317 | 0.007661979 | 0.005032371 | 0 | 0 |
| 83453 | Chrdl1 | −0.00024274 | −0.00297206 | −0.00703673 | −0.00566341 | −0.00518782 | 0 | 0 |
| 56722 | Litaf | 0.001830096 | 0.003130721 | 0.007035815 | 0.006529704 | 0.004300318 | 0 | 0 |
| 104158 | Ces1d | 0.0014056 | −2.56E−05 | 0.006957785 | 0.007393361 | 0.00629287 | 0 | 0 |
| 72281 | Sh2d4a | 0.001853033 | −0.00222817 | 0.006937475 | 0.005098914 | 0.003738592 | 0 | 0 |
| 14293 | Fpr1 | −0.00170524 | −0.0008335 | −0.00686254 | −0.00640543 | −0.00246374 | 0 | 0 |
| 13056 | Cyb561 | −0.00198034 | −0.00284016 | −0.00684817 | −0.00476749 | −0.00394581 | −0.02007018 | 0.060266172 |
| 236604 | Plsd-ps1 | 0.001960225 | 0.00087613 | 0.006748589 | 0.006861199 | 0 | 0 | 0 |
| 380997 | Cyp2d12 | 0 | 0.003196158 | 0.00673095 | 0 | 0 | 0 | 0 |
| 246049 | Slc36a2 | −0.00153067 | −2.06E−05 | −0.00672483 | −0.00881816 | −0.00561307 | 0 | 0 |
| 211945 | Plekhh1 | 0.001588353 | 0.003329741 | 0.006689505 | 0.007202535 | 0.003893795 | 0 | 0 |
| 12759 | Clu | 6.71E−05 | −0.00086682 | −0.00661607 | −0.00309519 | −0.00089292 | 0 | 0 |
| 13024 | Ctla2a | 0.001139691 | 0.003117413 | 0.006576751 | 0.007704071 | 0.004200617 | 0 | 0 |
| 11501 | Adam8 | 0.000617628 | −0.00329037 | −0.00657488 | −0.00648717 | −0.00203072 | 0 | 0.051871351 |
| 239126 | C1qtnf9 | −0.00082719 | 0 | −0.00657004 | −0.00217624 | 0 | 0 | 0 |
| 98238 | Lrrc59 | −0.00077161 | −6.11E−05 | −0.00656226 | −0.00770127 | −0.00044676 | 0 | 0 |
| 236285 | Land3 | 0.000465903 | −0.00016469 | 0.006457527 | 0.007453607 | 0.004262318 | 0 | 0 |
| 107371 | Exoc6 | 0.001370653 | 0.001138559 | 0.006438938 | 0.005922807 | 0.003347935 | 0 | 0 |
| 14239 | Foxs1 | 0.000508448 | 0.002010056 | 0.006341617 | 0.006844516 | 0.004700411 | 0 | 0 |
| 237175 | Adgrg2 | 0.000365447 | −0.00154911 | −0.00625644 | −0.00414477 | −0.00328648 | 0 | 0 |
| 12143 | Blk | 0.00169726 | 0.003041594 | 0.006216855 | 0.008088629 | 0.004554167 | 0 | 0 |
| 230991 | Fndc10 | 0.001987464 | 0.002389966 | 0.006209578 | 0.006584669 | 0.003243736 | 0 | 0 |
| 53897 | Gal3st1 | 0.000770712 | 0.002833016 | 0.006197328 | 0.006486448 | 0.00284672 | 0 | −0.05504643 |
| 16403 | Itgs6 | −0.00106419 | −0.00382395 | −0.00618226 | −0.0066649 | −0.00411719 | 0 | 0 |
| 109151 | Chd9 | −0.00084925 | −0.00163817 | −0.00612685 | −0.00574715 | −0.00321013 | 0 | 0 |
| 29816 | Hip1r | 0.000849051 | 0.002575591 | 0.006118887 | 0.005281704 | 0.004381211 | 0 | 0 |
| 15439 | Hp | −0.00149458 | −0.00223464 | −0.00611104 | −0.00466562 | −0.00166812 | 0 | 0.09447138 |
| 20556 | Slfn2 | −0.0013326 | 0.000624574 | −0.00609542 | −0.00520577 | −0.00451094 | 0 | 0 |
| 71242 | Spata24 | 0.00021322 | −0.00337986 | −0.00606859 | −0.00895281 | −0.00545465 | 0 | 0 |
| 215418 | Csrnp1 | 0.000782515 | 0.002436425 | 0.006054636 | 0.005807199 | 0.003386133 | 0 | −0.06774809 |
| 103768 | Tubg2 | −0.00083831 | −1.16E−05 | −0.00602381 | −0.00632146 | −0.00211504 | 0 | 0 |
| 19419 | Rasgrp1 | −0.00014433 | −0.00382862 | −0.00602049 | −0.00707108 | −0.00376954 | 0 | 0.119828996 |
| 106840 | Unc119b | 0.00064502 | 0.002119688 | 0.005921525 | 0.005755233 | 0.002724977 | 0 | 0 |
| 14071 | F9 | 0.001594803 | 0.003358795 | 0.005914282 | 0.007460654 | 0.003441407 | 0 | 0 |
| 23965 | Tenm3 | 0.000746992 | 0.000113898 | 0.005881573 | 0.005534734 | 0.00380312 | 0 | 0 |
| 12504 | Cd4 | 0.000168509 | −0.00114661 | −0.0058671 | −0.00025454 | −0.00212122 | 0 | 0 |
| 67739 | Slc48a1 | −0.00078085 | −0.00300494 | −0.00586229 | −0.00597699 | −0.00392996 | 0 | 0 |
| 319909 | Ism1 | −0.00167024 | 0 | −0.0058568 | −0.00622103 | −0.00423963 | 0 | 0 |
| 12402 | Cbl | −0.00074203 | 0.000922927 | −0.00585004 | −0.00455884 | −0.00432119 | 0 | 0 |
| 628308 | Zfp970 | 0 | 0.00082607 | 0.005846526 | 0 | 0 | 0 | 0 |
| 53972 | Ngef | −0.00123804 | 0.002092686 | −0.00584069 | −0.00580274 | −0.00315962 | 0 | 0.012452924 |
| 279028 | Adamts13 | −0.00074311 | −0.00332819 | −0.00583191 | −0.00320972 | −0.00163566 | 0 | 0 |
| 218629 | Dhx29 | −0.00120039 | 0.000912131 | −0.00582878 | −0.00685044 | −0.00423825 | 0 | 0 |
| 17427 | Mns1 | 0.000802886 | 0.00134231 | 0.005823218 | 0.006932656 | 0.004467791 | 0 | 0 |
| 81909 | Zfpl1 | 0.000154687 | 0.002761131 | 0.005804158 | 0.006325152 | 0.002033389 | 0 | 0 |
| 620078 | C130026I21Rik | 0 | 0.002648511 | 0.005802922 | 0 | 0 | 0 | −0.12882399 |
| 21898 | Tlr4 | 0.001075731 | 0.001300991 | 0.00579939 | 0.007275261 | 0.006422583 | 0 | 0 |
| 19179 | Psmc1 | 0.000609916 | 0.001499318 | 0.005757867 | 0.005453624 | 0.005889961 | 0 | 0 |
| 52679 | E2f7 | 0.001171914 | 0.002098482 | 0.005748095 | 0.006268516 | 0.004021626 | 0 | 0.006379043 |
| 16509 | Kcne1 | −0.00111167 | −0.00023976 | −0.00574017 | −0.00689762 | 0 | 0 | 0 |
| 15516 | Hsp90ab1 | −0.00082846 | −0.00178841 | −0.00570881 | −0.0048215 | −0.00262548 | 0 | 0 |
| 320183 | Msrb3 | 0.001359152 | 0.001245538 | 0.00569849 | 0.005597006 | 0.003443038 | 0 | 0 |
| 217826 | Kcnk13 | −0.00078558 | −0.00183047 | −0.00569798 | −0.00560901 | −0.00317099 | 0 | 0 |
| 69068 | Tcim | −0.00115592 | −0.00045392 | −0.00568944 | −0.00644117 | −0.00269432 | 0 | 0 |
| 22701 | Zfp41 | −0.00179595 | −0.00183058 | −0.00568428 | −0.00615671 | −0.00402128 | 0 | 0 |
| 12580 | Cdkn2c | 0.000858038 | 0.000708112 | 0.005672856 | 0.005252353 | 0.000827364 | 0 | 0 |
| 237222 | Ofd1 | 0.001318053 | 0.002923703 | 0.005670493 | 0.006764111 | 0.003303381 | 0 | −0.01009876 |
| 18946 | Pnliprp1 | −0.00010693 | 0.00134272 | 0.005664114 | 0.005883718 | 0.002831381 | 0 | 0 |
| 17068 | Ly6d | −0.00086966 | −0.00178071 | −0.00565065 | −0.00540018 | −0.00398312 | 0 | 0 |
| 93879 | Pcdhb8 | 0.000822074 | 0.001229839 | 0.005640347 | 0.006997733 | 0 | 0 | 0 |
| 19354 | Rac2 | −0.00013573 | −0.00120707 | −0.00563976 | −0.0038961 | −0.00131833 | 0 | 0 |
| 19220 | Ptgfr | 0.000708088 | −0.000793 | 0.005638423 | 0.003888501 | 0.002606257 | 0 | 0 |
| 67371 | Gtf3c6 | 6.34E−05 | −0.00098829 | −0.00563528 | −0.0062206 | −0.0006512 | 0 | 0 |
| 72141 | Adpgk | −0.0006901 | −0.00180749 | −0.00562635 | −0.0038888 | −0.00243123 | 0 | 0 |
| 231207 | Cpeb2 | 0.001045327 | 0.001640415 | 0.00562016 | 0.005479957 | 0.004982574 | 0 | 0 |
| 78913 | Ltn1 | −0.00094009 | −0.00195933 | −0.00559298 | −0.00574012 | −0.00479205 | 0 | 0 |
| 13447 | Doc2b | 0.001129332 | 0.000295178 | 0.005578017 | 0.002293703 | 0.003320691 | 0 | 0 |
| 12368 | Casp6 | −0.00041169 | −0.00048561 | −0.00557601 | −0.00581378 | −0.00436142 | 0 | 0 |
| 71326 | Treml1 | 0 | 0 | −0.00555508 | 0 | −0.00460306 | 0 | 0 |
| 237553 | Trhde | 0.000819463 | 0.001162411 | 0.005543815 | 0.006174162 | 0.000535339 | 0 | −0.24875278 |
| 69149 | Kbtbd3 | −0.00094766 | 0.000116348 | −0.00554371 | −0.00478291 | −0.00435115 | 0 | 0 |
| 17907 | Mylpf | 0.000303537 | 0.002893924 | 0.005543431 | 0.002029886 | 0.000725696 | 0 | −0.0113834 |
| 84004 | Mcam | 0.002000514 | 0.000264012 | 0.005539491 | 0.006412317 | 0.007143613 | 0 | 0 |
| 114642 | Brdt | −0.0010763 | −0.00135149 | −0.00553188 | −0.004495 | −0.00243462 | 0 | 0 |
| 23853 | Def6 | −0.00112057 | 0.002489758 | −0.0055223 | −0.00684522 | −0.00490195 | 0 | 0 |
| 57385 | P2ry4 | −0.00045401 | 0.002069219 | −0.00550683 | −0.00666766 | −0.00311677 | 0 | 0 |
| 14961 | H2-Ab1 | 0.000589238 | −0.00394788 | −0.00548134 | 0.000230253 | 0.002263322 | 0 | 0.100529678 |
| 23917 | Impdh1 | −0.00074385 | −0.00055948 | −0.00546095 | −0.00479605 | −0.00208996 | 0 | 0 |
| 545486 | Tubb1 | 0 | 0.00125293 | 0.00545988 | 0 | 0.003037416 | 0 | 0 |
| 68695 | Hddc3 | −0.0001042 | −0.00245844 | −0.00545568 | −0.00468571 | −0.00113796 | 0 | 0 |
| 140742 | Sesn1 | −0.00122557 | −0.00120938 | −0.00544958 | −0.00536567 | −0.00221354 | 0 | 0 |
| 12854 | Cort | 0 | −0.00217682 | −0.00543788 | 0 | −0.0057099 | 0 | 0 |
| 216551 | Lgalsl | −0.00044308 | −0.00119287 | −0.0054356 | −0.00522527 | −0.00175255 | 0 | 0 |
| 21411 | Tcf20 | −0.00167507 | −0.00255319 | −0.00542983 | −0.00409522 | −0.00388204 | 0 | 0 |
| 242594 | Fyb2 | 0 | −0.00371254 | −0.00542239 | 0 | −0.001402 | 0 | 0 |
| 18514 | Pbx1 | 0.001111066 | 0.001186459 | 0.005420228 | 0.005044977 | 0.003038658 | 0 | 0 |
| 321019 | Gpr183 | 0.00082943 | 0 | 0.005415389 | 0.00433279 | 0.00203844 | 0 | 0 |
| 20378 | Frzb | 0.000779316 | 0.002641554 | 0.005409444 | 0.005286083 | 0.004472244 | 0 | 0 |
| 230484 | Usp1 | 0.000545235 | 0.001265867 | 0.005407229 | 0.004388044 | 0.003143678 | 0 | 0 |
| 56213 | Htra1 | −0.00142355 | −0.0032387 | −0.00539305 | −0.00531775 | −0.00225421 | 0 | 0 |
| 240023 | Pnldc1 | 0.000731404 | 0.000676819 | 0.005382458 | 0.003535861 | 0.002935525 | 0 | −0.1116202 |
| 171180 | Syt12 | −0.00160646 | −0.00263816 | −0.0053812 | −0.00647254 | −0.00511944 | 0 | 0 |
| 54672 | Adgrg3 | −0.00010429 | −0.00359915 | −0.00535671 | −0.00779388 | −0.00274213 | 0 | 0 |
| 14255 | Flt3 | −0.00134785 | −0.00026967 | −0.00533282 | −0.00495451 | −0.00382395 | 0 | 0 |
| 327826 | Frs2 | −0.00055192 | −0.0002898 | −0.00532815 | −0.00531307 | −0.00103731 | 0 | 0 |
| 20649 | Sntb1 | −0.00094334 | 0.000432941 | −0.0053215 | −0.00400695 | −0.00113967 | 0 | 0 |
| 76464 | Kni1 | 0.002004378 | 0 | 0.005319462 | 0.006256445 | 0.003627103 | 0 | 0 |
| 70945 | Mmrn1 | 8.16E−05 | 0 | −0.00531492 | −0.00641415 | −0.00212672 | 0 | 0 |
| 83962 | Btbd1 | 0.00170451 | 0.000166839 | 0.005313389 | 0.006219052 | 0.004763939 | 0 | 0 |
| 259300 | Ehd2 | −0.00081535 | −0.00150699 | −0.00529996 | −0.00537865 | −0.00171211 | 0 | 0 |
| 231287 | Atp10d | −0.00132755 | −0.00175582 | −0.00529297 | −0.00458645 | −0.00251766 | 0 | 0 |
| 76566 | Rfinb | −0.0010299 | −0.00130582 | −0.00528871 | −0.00609786 | −0.003177 | 0 | 0 |
| 240752 | Pik3c2b | 0.001236384 | 0.001589052 | 0.005274337 | 0.00450629 | 0.003357255 | 0 | 0 |
| 112422 | Zfp979 | 0.000519592 | 0.001244669 | 0.005261136 | 0.004745664 | 0 | 0 | 0 |
| 268480 | Rapgefl1 | −0.00158271 | −0.00178585 | −0.00526025 | −0.00582281 | −0.00469802 | 0 | 0 |
| 57430 | Sult3a1 | −0.00116075 | −0.0037975 | −0.00524402 | −0.00272667 | 0 | 0 | 0 |
| 226153 | Twnk | −0.00137215 | 0.000300126 | −0.00523852 | −0.00505829 | −0.00402739 | 0 | 0 |
| 69836 | Pla2g12b | 0.001479937 | −0.00041202 | 0.005237988 | 0.005098389 | 0.003335456 | 0 | 0 |
| 110935 | Atp6v1b1 | −0.00071802 | −0.0018268 | −0.00522787 | −0.00215697 | −0.00113497 | 0 | 0 |
| 73666 | Thoc3 | 0.001639911 | 0.000654836 | 0.005222221 | 0.00437177 | 0.003130455 | 0 | 0 |
| 218460 | Wdr41 | −0.00077263 | 9.42E−05 | −0.00521777 | −0.00445162 | −0.00431007 | 0 | 0 |
| 269152 | Kif26b | 1.29E−05 | 0.000676661 | 0.005198433 | 0.003111417 | 0.001140048 | 0 | 0 |
| 14680 | Gnal | 0.000347591 | −0.00044812 | 0.005190966 | 0.00495642 | 0.002322578 | 0 | 0 |
| 14763 | Gpr37 | −0.0001717 | 0.001318279 | 0.005178862 | 0.00463613 | 0.003230403 | 0 | 0 |
| 21412 | Tcf21 | −0.00105784 | −0.00106129 | −0.00517518 | −0.00328383 | −0.00205718 | 0 | 0.131126201 |
| 16779 | Lamb2 | −0.00085635 | −0.0024994 | −0.00517413 | −0.00587682 | −0.00386595 | 0 | 0 |
| 21825 | Thbs1 | 0.000379869 | −0.0039255 | 0.005173333 | 0.002163507 | 0.003144745 | 0 | 0 |
| 67629 | Spc24 | −0.00110737 | −0.0002927 | −0.00516616 | −0.00300801 | −0.00184431 | 0 | 0 |
| 13345 | Twist2 | 0.001045422 | 0 | 0.005165406 | 0.00169023 | 0.00228131 | 0 | 0 |
| 26944 | Tinag | −0.00087121 | −0.0033826 | −0.00515893 | −0.00413687 | −0.00170334 | 0 | 0 |
| 13035 | Ctsg | 0.001560083 | 0.003223262 | 0.005157701 | 0.006206071 | 0.002394501 | 0 | 0 |
| 621080 | Al429214 | −0.00109001 | −0.00333287 | −0.00515769 | −0.00497354 | 0 | 0 | 0.064101157 |
| 16456 | F11r | 0.000983692 | 0.001821704 | 0.005152409 | 0.004593817 | 0.003229617 | 0 | 0 |
| 14187 | Akr1b8 | 0.001027234 | 0.002371165 | 0.005143583 | 0.005129011 | 0.003276465 | 0 | −0.02646969 |
| 67216 | Mboat2 | −0.00183657 | −0.00064071 | −0.00514299 | −0.00403 | −0.00261428 | 0 | 0 |
| 271564 | Vps13a | 0.001644122 | 0.000201949 | 0.005139009 | 0.004779058 | 0.00317887 | 0 | 0 |
| 14190 | Fgl2 | 9.79E−05 | 0.001139224 | −0.00513383 | −0.00335868 | −0.0010101 | 0 | 0 |
| 13371 | Dio2 | −0.00102608 | 0.000714841 | −0.00512985 | −0.00563001 | −0.00320302 | 0 | 0 |
| 21390 | Tbxa2r | −0.00159343 | −9.62E−05 | −0.00512157 | −0.00465026 | −0.00302583 | 0 | 0 |
| 72789 | Veph1 | 0.000797966 | −0.00191996 | −0.0051215 | −0.00487896 | −0.00294785 | 0 | 0 |
| 76976 | Arxes2 | 0 | −0.00063783 | −0.00511468 | 0 | 0 | 0 | 0 |
| 27883 | Tango2 | −0.00086819 | 0.000309843 | −0.00510939 | −0.0047933 | −0.0020801 | 0 | 0.042341322 |
| 667370 | Ifit3b | 0.000911962 | 0.001591359 | 0.005104433 | 0.005001604 | 0 | 0 | 0 |
| 16534 | Kcnn4 | 0.000894237 | −0.0014458 | −0.00510216 | −0.00465654 | 0.00171865 | 0 | 0 |
| 68490 | Zfp579 | 0.000456778 | 0.001948626 | 0.005099911 | 0.00499935 | 0.002067829 | 0 | 0 |
| 67445 | C1qtnf4 | 0.001669363 | 0.000757291 | 0.005098832 | 0.005259489 | 0.0023987 | 0 | 0 |
| 22151 | Tubb2a | −0.00159528 | −0.00186709 | −0.0050982 | −0.00766654 | −0.00265083 | 0 | 0 |
| 15376 | Foxa2 | −0.00025091 | −0.00116693 | −0.00509086 | −0.00360603 | −0.00135712 | 0 | 0 |
| 27384 | Akr1c13 | 0.000551773 | −0.00059644 | 0.005090746 | 0.003944188 | 0 | 0 | 0 |
| 210503 | Zfp677 | 0.000713256 | 0 | 0.005086268 | 0.004717184 | 0 | 0 | 0 |
| 12458 | Ccr6 | −0.0005067 | −0.00017965 | −0.00508052 | −0.00659541 | −0.00256934 | 0 | 0 |
| 83814 | Nedd4l | 0.00058563 | −0.00254289 | −0.00507924 | −0.00441941 | −0.00226245 | 0 | 0 |
| 271786 | Galnt13 | −4.29E−05 | 0 | −0.00190076 | 0.011318678 | 0.002924648 | 0 | 0 |
| 64085 | Clstn2 | 0.000357267 | 0.000412633 | −0.00043786 | 0.008549894 | 0.004780052 | 0 | 0.051146301 |
| 14134 | Fcnb | 0.000349573 | 0 | 0 | −0.00820368 | −0.00062891 | 0 | 0 |
| 110902 | Chrna2 | −0.00106679 | −0.00279037 | −0.00501474 | −0.00768849 | −0.00496801 | 0 | 0.045981656 |
| 243912 | Hspb6 | −0.0006228 | −0.00259682 | −0.00319577 | −0.00767534 | −0.00201001 | 0 | 0 |
| 105298 | Epdr1 | −0.00047648 | −0.00301957 | −0.00411858 | −0.00755863 | 8.11E−05 | 0 | 0.082608903 |
| 67389 | C1qtnf12 | 0.001031609 | 0.000763064 | 0.00502692 | 0.007178606 | 0.005100321 | 0 | 0 |
| 14938 | Gzma | −0.00126089 | −0.00395684 | 0 | −0.00711337 | −0.00446728 | 0 | 0 |
| 11535 | Adm | 0.001353525 | 0.002912502 | 0.003991959 | 0.007096059 | 0.004467665 | 0 | 0 |
| 14394 | Gabra1 | −0.00025892 | −0.0006037 | −0.0022035 | −0.00707774 | −0.00290974 | 0 | 0 |
| 229003 | Helz2 | 0.000237372 | −0.00018729 | −0.00359069 | −0.0070336 | −0.00340285 | 0 | 0 |
| 228775 | Trib3 | −0.0010443 | −0.00071222 | −0.00277804 | −0.00702612 | −0.00301606 | 0 | 0 |
| 20295 | Ccl17 | −0.00059368 | −0.00087501 | −0.00081357 | −0.00701 | −0.00231337 | 0 | 0 |
| 192113 | Atp12a | −0.00093153 | 0.000621342 | −0.00218964 | −0.0068553 | −0.00233249 | 0 | 0 |
| 12500 | Cd3d | 0.000591935 | 0.001744431 | 0.004252788 | 0.006802304 | 0.003725788 | 0 | 0 |
| 52184 | Odf2l | 0.000959522 | 0.003086434 | 0.004650195 | 0.006776478 | 0.004147686 | 0 | 0 |
| 13608 | Edar | −0.00053773 | −0.00122878 | 0 | −0.0067684 | −0.00328338 | 0 | 0 |
| 94279 | Sfxn2 | 0.000184329 | 0.003357094 | 0.003568632 | 0.006758435 | 0.004351727 | 0 | −0.06201351 |
| 20964 | Syn1 | −0.00118255 | 0 | −0.00280844 | −0.00665038 | −0.00345763 | 0 | 0 |
| 18619 | Penk | −0.00024847 | 0 | −0.0010411 | −0.00657204 | 0.001087401 | 0 | 0 |
| 207911 | Mchr1 | 4.80E−05 | 0 | 0.000999119 | 0.00656406 | 0.003196866 | 0 | 0 |
| 109594 | Lmo1 | −0.001391 | −0.00170123 | −0.00494222 | −0.0064976 | −0.00235821 | 0 | 0 |
| 68498 | Tspan11 | −0.00153328 | −3.21E−05 | −0.00170305 | −0.00643565 | −0.00238224 | 0 | 0 |
| 70377 | Derl3 | 0.001820413 | 0.000472791 | 0.005062099 | 0.006427054 | 0.005116865 | 0 | −0.10594567 |
| 98363 | Efhd1 | 0.000880493 | 0.001998983 | 0.004610405 | 0.006425082 | 0.002983504 | 0 | 0 |
| 12023 | Barx2 | 0.00128427 | −0.00146589 | 0.004824228 | 0.006369024 | 0.005244215 | 0 | 0 |
| 170752 | Bco2 | 0.001773377 | 0.003156701 | 0.004538931 | 0.00635018 | 0.004690733 | 0 | −0.02085612 |
| 329366 | Ccdc187 | −0.00155176 | −0.00083639 | −0.00391233 | −0.00629589 | −0.00309241 | 0 | 0 |
| 14696 | Gnb4 | −0.00198301 | −0.00024636 | −0.00247523 | −0.00628625 | −0.00230961 | 0 | 0 |
| 72169 | Trim29 | −0.00040304 | −0.00012746 | −0.00106266 | −0.00626084 | −0.00123018 | 0 | 0 |
| 73804 | Klf2c | 0.000697436 | −0.00042999 | 0.001585134 | 0.006222116 | 0.0017231 | 0 | 0 |
| 20410 | Sorbs3 | 0.001735048 | 0.002303358 | 0.005047361 | 0.006216008 | 0.003521061 | 0 | −0.05852705 |
| 78284 | Creb3l4 | −0.00209337 | −8.44E−05 | −0.00353249 | −0.00614458 | −0.00415324 | 0 | 0 |
| 17130 | Smad6 | 0.001303155 | 0.003101144 | 0.004251603 | 0.006143998 | 0.004076151 | 0 | 0 |
| 58222 | Rab37 | 2.15E−05 | −0.00118669 | −0.00354417 | −0.00609908 | −0.00276604 | 0 | 0 |
| 17474 | Clec4d | 0.001672448 | 0.001061827 | 0 | −0.00609464 | −0.00081647 | 0 | 0 |
| 16783 | Lamp1 | 0.001109607 | 0.001425818 | 0.005075832 | 0.006062203 | 0.005354493 | 0 | 0 |
| 18439 | P2rx7 | 0.001079437 | 0.001885479 | 0.004008954 | 0.006052639 | 0.001991623 | 0 | 0 |
| 18969 | Pola2 | −0.00129022 | −0.00228682 | −0.00392285 | −0.00604294 | −0.00287187 | 0 | 0 |
| 243374 | Gimap8 | −0.00063221 | 0.001449831 | −0.00431207 | −0.0060224 | −0.00390068 | 0 | 0 |
| 11576 | Afp | 0.000250151 | 0.001805303 | 0.003794981 | 0.00601041 | 0.004279518 | 0 | 0.01097634 |
| 16002 | Igf2 | 0.001954101 | −0.00071625 | 0.002747072 | 0.005978917 | 0.005427368 | 0 | 0 |
| 14129 | Fcgr1 | 0.00161035 | −0.00199342 | 0.001577592 | 0.00596554 | 0.00399726 | 0 | 0 |
| 75590 | Dusp9 | −0.00114834 | −0.00049566 | −0.004115 | −0.00595009 | −0.00311589 | 0 | 0 |
| 381319 | Batf3 | 0.00016194 | −0.00347827 | −0.00350667 | −0.00591583 | −0.00275422 | 0 | 0 |
| 14635 | Galk1 | −0.00142347 | −0.00213814 | −0.00460689 | −0.00590416 | −0.00374713 | 0 | 0 |
| 11720 | Mat1a | −0.00063837 | 0.001366119 | 7.18E−05 | −0.00588862 | −0.00366114 | 0 | 0 |
| 108897 | Aif1l | 0.001857886 | 0.002508401 | 0.003932271 | 0.005869943 | 0.001970249 | 0 | 0 |
| 210673 | Prrt3 | 0.000387854 | 0.001258682 | 0.004059582 | 0.00586723 | 0.000848072 | 0 | 0 |
| 72434 | Lypd3 | −0.0008946 | −0.00162093 | −0.00425876 | −0.00586403 | −0.00277072 | 0 | 0 |
| 238988 | Erc2 | 0.001991896 | −0.00216941 | 0.004544811 | 0.005863709 | 0.004427608 | 0 | 0 |
| 14205 | Vegfd | 0.000838261 | −0.00037885 | 0.002665689 | 0.005860302 | 0.005253473 | 0 | 0 |
| 384009 | Glipr2 | 0.000859829 | −0.00018837 | 0.003010676 | 0.005853467 | 0.001913696 | 0 | 0 |
| 73720 | Cst6 | 0.000506448 | −0.00111773 | −0.00406484 | −0.00585313 | −0.00237922 | 0 | 0 |
| 20191 | Ryr2 | 0.001034808 | 0.003200676 | 0.004579951 | 0.005829638 | 0.005030508 | 0 | 0 |
| 16775 | Lama4 | −0.00178469 | −0.00374412 | −0.00502447 | −0.00581298 | −0.00395266 | 0 | 0 |
| 67486 | Polr3g | 0.001310368 | 0.002493429 | 0.004379518 | 0.005805147 | 0.002974015 | 0 | 0 |
| 66431 | Oxld1 | 0.0004669 | 0.000968225 | 0.003893223 | 0.005804167 | 0.00379966 | 0 | 0 |
| 243914 | Lgi4 | −0.0017655 | −0.00014374 | −0.00308682 | −0.00580243 | −0.00348047 | 0 | 0 |
| 76187 | Adhfe1 | −0.0009884 | −0.00192163 | −0.00490476 | −0.00579179 | −0.00157503 | 0 | 0 |
| 74195 | Elp3 | 0.001282841 | 0.001352118 | 0.00498082 | 0.005774699 | 0.004236186 | 0 | 0 |
| 232801 | Lilra5 | −0.00035643 | −0.00146401 | −0.00278536 | −0.00576067 | −0.00245968 | 0 | 0 |
| 226025 | Trpm3 | −0.00096118 | −0.00095114 | −0.00388243 | −0.00575695 | −0.0022561 | 0 | 0 |
| 217125 | Samd14 | 0.001462085 | 0.001874898 | 0.003386657 | 0.005751997 | 0.002942081 | 0 | 0 |
| 16402 | Itga5 | 0.001276183 | 6.31E−05 | 0.003982137 | 0.005741543 | 0.004767429 | 0 | 0 |
| 14809 | Grik5 | −0.00058873 | −0.00061986 | −0.00342837 | −0.00573888 | −0.00235156 | 0 | 0 |
| 18019 | Nfatc2 | −0.00065556 | −0.00150801 | −0.0029664 | −0.00572185 | −0.00282561 | 0 | 0 |
| 214685 | Chadl | 0.001203629 | 0 | 0.003362533 | 0.005719702 | 0.002859214 | 0 | 0 |
| 11606 | Agt | −0.00118862 | 0.001247254 | −0.00154006 | −0.00571189 | −0.00228788 | 0 | 0 |
| 18034 | Nfkb2 | 0.001147171 | 0.001390177 | 0.002190719 | 0.005710917 | 0.002671036 | 0 | 0 |
| 224674 | Slc37a1 | −0.00146104 | −0.00346936 | −0.00362937 | −0.00570841 | −0.00231719 | 0 | 0 |
| 75538 | Gerin5a | −0.00046884 | 0.001525726 | −0.00390965 | −0.0057024 | −0.00407646 | 0 | 0 |
| 72415 | Sgo1 | 0.000462653 | 0.000400541 | 0.001703778 | 0.005678164 | 0.001344499 | 0 | 0 |
| 665270 | Plb1 | −0.0001923 | 0.00031266 | 0.003334381 | 0.005677212 | 0.001903476 | 0 | 0 |
| 94332 | Cadm3 | 0.000231209 | −0.00275557 | −0.00363861 | −0.00566845 | −0.00269527 | 0 | 0 |
| 68612 | Ube2c | −0.00187664 | −2.40E−06 | −0.00506003 | −0.00566587 | −0.00569145 | 0 | 0 |
| 14401 | Gabrb2 | −0.00079915 | 0 | 0 | −0.00565549 | −0.00338084 | 0 | 0 |
| 14673 | Gna12 | 0.000395129 | 0.002918377 | 0.002945901 | 0.005645897 | 0.0031102 | 0 | 0 |
| 11504 | Adamts1 | 0.001513012 | 0.000977868 | 0.003970788 | 0.005638972 | 0.00129037 | 0 | 0 |
| 71756 | Cpn2 | 7.98E−05 | −0.00322267 | 0.005037845 | 0.005638967 | 0.004071124 | 0 | 0 |
| 19885 | Rorc | 0.000701963 | −0.00025937 | 0.004662314 | 0.005617411 | 0.003300743 | 0 | 0.202083252 |
| 19240 | Tmsb10 | −0.00055879 | −0.00113959 | −0.00483942 | −0.00560789 | −0.00410418 | 0 | 0 |
| 54396 | Irgm2 | 0.000278411 | −0.00201389 | −0.00449756 | −0.00560534 | −0.00153459 | 0 | −0.00551471 |
| 70337 | Iyd | 0.001816216 | 0.000793714 | 0.004882431 | 0.005599489 | 0.003570216 | 0 | 0 |
| 18476 | Pafah1b3 | 0.001158046 | 0.001398121 | 0.002390902 | 0.005590492 | 0.002516887 | 0 | 0 |
| 214058 | Megf11 | 6.58E−05 | −0.00247772 | −0.00442822 | −0.00558064 | −0.00405336 | 0 | 0 |
| 67928 | Abca14 | −0.00080929 | 0.001259265 | −0.00085537 | −0.00557995 | 0 | 0 | 0 |
| 110521 | Hivep1 | −0.00105504 | −0.00056205 | −0.00444922 | −0.00557885 | −0.00300009 | 0 | 0 |
| 11790 | Speg | 0.000183215 | −0.00034361 | 0.003751344 | 0.005549639 | 0.00106536 | 0 | 0.017394743 |
| 68854 | Asb11 | 0.000579239 | 0.000522987 | 0.004867097 | 0.005539311 | 0.003797429 | 0 | 0 |
| 171284 | Timd2 | 0.000477561 | −0.00018704 | 0.002315705 | 0.005527926 | 0.002902352 | 0 | 0 |
| 17089 | Lyar | −0.00165954 | −0.00259686 | −0.00466721 | −0.00552696 | −0.00306928 | 0 | 0 |
| 14615 | Gjc1 | −0.00096646 | −0.00192259 | −0.00438067 | −0.00552675 | −0.00220463 | 0 | 0 |
| 13417 | Dnah8 | −0.00018792 | −0.00203918 | −0.00376361 | −0.00552633 | −0.00320115 | 0 | 0 |
| 71233 | Enkur | 0.001249915 | 0 | 0.001247075 | 0.005505175 | 0.001735482 | 0 | 0 |
| 238076 | Kcns3 | −0.00030349 | −0.00321127 | −0.00347986 | −0.00550349 | −0.00447956 | 0 | 0 |
| 52202 | Rbm34 | 0.001596119 | 0.001593078 | 0.00415691 | 0.005502831 | 0.003641495 | 0 | 0 |
| 13821 | Epb41l1 | −0.00076328 | −0.00177218 | −0.00380825 | −0.0054985 | −0.00488084 | 0 | 0.087748551 |
| 15483 | Hsd11b1 | −0.00031168 | −0.00194717 | −0.00119468 | 0.000142349 | 0.00826708 | 0 | 0 |
| 22786 | Zp1 | 0 | 0 | 0 | 0 | 0.008008352 | 0 | 0 |
| 545192 | Baiap3 | 0.001266817 | 0 | 0.003911262 | 0.002671577 | 0.007168417 | 0 | 0 |
| 16669 | Krt19 | −0.00195796 | −0.00098485 | −0.00273595 | −0.00417906 | −0.00656057 | 0 | 0 |
| 75645 | Ccdc172 | 0 | 0 | 0 | 0 | −0.00651324 | 0 | 0 |
| 54561 | Nap1l3 | 0.001869896 | 0.001386009 | 0.002789498 | 0.002954549 | 0.006281707 | 0 | 0 |
| 106878 | Smim3 | −0.00135718 | −0.00121708 | −0.00463603 | −0.00510409 | −0.00623472 | 0 | 0 |
| 19073 | Srgn | −0.00045585 | −0.00079498 | −0.00137527 | −0.00420929 | −0.00576961 | 0 | 0 |
| 72014 | Btbd17 | −0.00030909 | −0.0005672 | −0.0024956 | −0.00154683 | −0.00553426 | 0 | 0 |
| 110446 | Acat1 | 0.001129766 | −0.00122109 | 0.002875999 | 0.004197664 | 0.005474066 | 0 | 0.026279019 |
| 18548 | Pcsk1 | 0.001178085 | 0 | 0.002295867 | 0.004112649 | 0.005462878 | 0 | 0 |
| 226830 | Smyd2 | 0.001142562 | −0.00094256 | 0.00379134 | 0.004189508 | 0.005460497 | 0 | 0 |
| 100038882 | Isg15 | 0.0005525 | 0.001934334 | −0.00238181 | −0.00048206 | −0.00543772 | 0 | 0 |
| 260301 | Otos | 0 | 0 | 0 | 0 | 0.005406004 | 0 | 0 |
| 11829 | Aqp4 | 0.001947997 | 0.000949988 | 0.001626271 | 0.003808514 | 0.005390626 | 0 | 0 |
| 68236 | Gtsf1l | 0 | 0.001416014 | 0 | 0 | 0.005315387 | 0 | 0 |
| 16170 | Il16 | −0.00146034 | −4.53E−05 | −0.00441513 | −0.00480139 | −0.00529889 | 0 | 0 |
| 17463 | Psmd7 | 8.27E−05 | −7.89E−05 | −0.00366751 | −0.00372943 | −0.00529685 | 0 | 0 |
| 12700 | Cish | −0.0012853 | 0.001194305 | −0.00439048 | −0.00381384 | −0.00529255 | 0 | 0 |
| 272428 | Acsm5 | −0.00010673 | 0.000110767 | 0.000928604 | 0.004657105 | 0.005210844 | 0 | 0 |
| 17380 | Mme | −0.0003684 | 0.001344599 | −0.00186954 | −0.00365847 | −0.00520007 | 0 | 0 |
| 225997 | Trpm6 | −0.00076056 | −0.00212186 | −0.00333101 | −0.00496772 | −0.0051739 | 0 | 0.007545214 |
| 12579 | Cdkn2b | 0.001163466 | 0.0004296 | 0.000119582 | 0.000883672 | 0.005165228 | 0 | 0 |
| 77113 | Klhl2 | −0.00125805 | 0.000520442 | −0.00432101 | −0.00441088 | −0.0050663 | 0 | 0 |
| 102774 | Bbs4 | 0.001821919 | 0.001609591 | 0.003661917 | 0.004484064 | 0.005051395 | 0 | 0 |
| 20888 | Sult1c1 | 0 | 0.000533461 | 0 | 0 | 0.00500585 | 0 | 0 |
| 16516 | Kcnj15 | 0.00169907 | 0.003441866 | 0.00394427 | 0.005333146 | 0.004957842 | 0 | −0.07682912 |
| 19085 | Prkar1b | 0.000490456 | 0.00181404 | 0.003996975 | 0.005028358 | 0.004938795 | 0 | 0 |
| 13385 | Dlg4 | −0.00194113 | 0.000861171 | −0.00369778 | −0.00370687 | −0.00491696 | 0 | 0.005068068 |
| 225845 | Plaat3 | −9.15E−05 | −0.00211654 | −0.00195242 | −0.00188907 | −0.0048941 | 0 | 0.028057981 |
| 14842 | Gsx1 | 0 | 0 | 0 | 0 | 0.004876622 | 0 | 0 |
| 17433 | Mobp | −0.00052901 | −0.00069772 | −0.00078162 | −0.00106709 | −0.00487455 | 0 | 0 |
| 67623 | Tm7sf3 | 0.00120841 | −0.00058285 | 0.002972553 | 0.004042431 | 0.004870903 | 0 | 0 |
| 80286 | Tusc3 | −0.00197736 | 9.53E−05 | −0.00413557 | −0.00435519 | −0.00485434 | 0 | 0 |
| 69564 | Nmrk2 | 0.001099802 | 0 | 0.003574337 | 0.00362256 | 0.004843016 | 0 | 0 |
| 20462 | Tra2b | 0.001213589 | 0.001085388 | 0.00210276 | 0.003080507 | 0.004804583 | 0 | 0 |
| 225579 | Slc27a6 | 0 | 0 | 0 | 0 | 0.004777559 | 0 | 0 |
| 20265 | Scn1a | 0.00053229 | 0 | −0.00069965 | 0.003614078 | 0.004775948 | 0 | 0 |
| 67245 | Peli1 | −0.00158759 | −0.00028851 | −0.00470391 | −0.00487537 | −0.00475312 | 0 | 0 |
| 13479 | Dpep1 | −0.00108836 | −0.00328435 | −0.00314868 | −0.00380496 | −0.00473082 | 0 | 0 |
| 21817 | Tgm2 | 0.001433547 | 0.000204907 | 0.003065293 | 0.004561055 | 0.004697227 | 0 | 0 |
| 26372 | Clcn6 | 0.001594425 | 0.002722233 | 0.002767753 | 0.004635708 | 0.004662095 | 0 | 0 |
| 56534 | Hspb3 | 0.000594001 | −0.0013301 | 0.000209353 | 0.00450205 | 0.004651419 | 0 | 0 |
| 74194 | Rnd3 | −0.00124939 | 0.001712005 | −0.00034538 | −0.00079524 | −0.00464695 | 0 | 0 |
| 56448 | Cyp2d22 | −0.0011022 | −0.00026024 | −0.00289125 | −0.00546986 | −0.00463539 | 0 | 0 |
| 20230 | Satb1 | −0.00149511 | −0.00072753 | −0.00105371 | −0.00417441 | −0.00462694 | 0 | 0 |
| 75599 | Pcdh1 | 0.001289304 | 0.002931024 | 0.00333437 | 0.004610015 | 0.004603158 | 0 | −0.01323324 |
| 56421 | Pfkp | 0.00062166 | 0.001600212 | 0.002296493 | 0.001806464 | 0.004581136 | 0 | 0 |
| 105171 | Arrdc3 | −0.00080019 | −0.00085768 | −0.00468883 | −0.00443529 | −0.00457069 | 0 | 0 |
| 12322 | Camk2a | −0.00076351 | 0.000536803 | −0.00249576 | −0.00496162 | −0.0045675 | 0 | 0 |
| 16504 | Kcnc3 | −0.00073198 | −0.00210442 | −0.00384775 | −0.00334805 | −0.00456546 | 0 | 0 |
| 232086 | Tmem150a | −0.00094093 | −0.00127146 | −0.00443816 | −0.00395272 | −0.00456339 | 0 | 0 |
| 228852 | Ppp1r16b | −0.00174687 | −0.00273835 | −0.00346563 | −0.00387155 | −0.00455796 | 0 | 0 |
| 20199 | S100a5 | 0 | 0 | 0 | 0 | 0.004554523 | 0 | 0 |
| 110310 | Krt7 | −0.00145066 | −0.00201763 | −0.00436725 | −0.00404461 | −0.00453741 | 0 | 0 |
| 630146 | Cd101 | 0 | 0 | 0 | 0 | 0.004531882 | 0 | 0 |
| 195209 | Zfp469 | 0 | 0 | 0 | 0 | 0.004507073 | 0 | 0 |
| 53602 | Hpcal1 | −0.00078017 | −0.00057971 | −0.00302467 | −0.00321217 | −0.00450361 | 0 | 0 |
| 108907 | Nusap1 | −0.00025793 | −0.00250446 | 0.003788346 | 0.002576912 | 0.004487963 | 0 | 0 |
| 66454 | Nmnat1 | −0.0016323 | −0.00150052 | −0.00448072 | −0.00504693 | −0.00448457 | 0 | 0 |
| 382571 | Kcnf1 | −0.00127924 | 0 | −0.00297895 | −0.00357782 | −0.00447912 | 0 | 0 |
| 17912 | Myo1b | −0.00180126 | −0.00221176 | −0.00432283 | −0.00481193 | −0.00446802 | 0 | 0 |
| 67856 | Echdc3 | −0.0015019 | −0.00016524 | −0.0030783 | −0.00387364 | −0.00446029 | 0 | 0 |
| 14991 | H2-M3 | 0.000846856 | 0.000712698 | 0.001306136 | 0.002967942 | 0.004451666 | 0 | 0 |
| 403187 | Opa3 | 0.000953557 | 0.001033939 | 0.004727101 | 0.005111873 | 0.004451193 | 0 | 0 |
| 66106 | Smpx | 0.000983166 | −0.00052084 | 0.003233834 | 0.003954191 | 0.004422212 | 0 | 0 |
| 229600 | BC028528 | 2.51E−05 | 0.000944566 | −0.0014982 | −0.00181371 | −0.00440936 | 0 | 0 |
| 23886 | Gdf15 | 0.002003596 | 0.000860554 | 0.00288959 | 0.002655794 | 0.00440625 | 0 | 0 |
| 13849 | Ephx1 | 0.001387651 | 0.000655737 | 0.001561557 | 0.003137896 | 0.004390873 | 0 | 0.04904025 |
| 13036 | Ctsh | 0.000923756 | 0.001569071 | 0.004769745 | 0.004136306 | 0.00438384 | 0 | 0 |
| 21809 | Tgfb3 | 0.001069172 | 0.00369696 | 0.00307977 | 0.005154679 | 0.004383813 | 0 | −0.05949772 |
| 109272 | Mybpc1 | 0.000541805 | −1.43E−05 | 0.000224252 | 0.004017802 | 0.004382697 | 0 | 0 |
| 15212 | Hexb | 0.001016794 | 0.003293819 | 0.002302893 | 0.0017179 | 0.004378305 | 0 | 0 |
| 76580 | Mib2 | −0.00119675 | −0.00345012 | −0.00409765 | −0.00459864 | −0.00437507 | 0 | 0 |
| 100072 | Camta1 | 0.00152738 | 0.000398894 | 0.002371793 | 0.003571861 | 0.004363784 | 0 | 0 |
| 67931 | Serpini2 | 0 | 0 | 0 | 0 | −0.00435937 | 0 | 0 |
| 12845 | Comp | 0.001089813 | 0.0008762 | 0.004044187 | 0.00387687 | 0.004355412 | 0 | 0 |
| 67724 | Pop1 | −0.00139702 | −0.00147108 | −0.00335511 | −0.00451204 | −0.00434018 | 0 | 0 |
| 22673 | Zfp185 | 0.000613094 | 0.001429078 | 0.004953209 | 0.005205922 | 0.004333246 | 0 | 0 |
| 77220 | Tmem200a | −0.00150355 | 0 | −0.00151453 | −0.00425217 | −0.00433158 | 0 | 0 |
| 27280 | Phlda3 | 0.001694667 | 0.001800066 | 0.00392643 | 0.002388346 | 0.00431203 | 0 | 0 |
| 79565 | Mettl27 | 0.000327765 | 6.93E−05 | 0.001952931 | 0.002968995 | 0.00430301 | 0 | 0 |
| 224860 | Plcl2 | −0.00066142 | 0.001557362 | −0.00234201 | −0.00446993 | −0.00429597 | 0 | 0 |
| 73737 | Bbin | −0.00161046 | −0.00225658 | −0.0039475 | −0.00350546 | −0.00427988 | 0 | 0 |
| 93888 | Pcdhb17 | 0.001521085 | 0.000399331 | 0.002083708 | 0.005018327 | 0.004278657 | 0 | 0 |
| 84111 | Gpr87 | 0 | −1.08E−05 | 0 | 0 | 0.004274815 | 0 | 0 |
| 110855 | Pde6c | −0.00085356 | −0.00013786 | −0.00413551 | −0.00443557 | −0.00426588 | 0 | 0.010332133 |
| 15450 | Lipc | 0.000582921 | −0.00279752 | 0.003979766 | 0.005099694 | 0.004265812 | 0 | 0 |
| 14308 | Fshb | 0 | 0.001890049 | 0 | 0 | 0.004254434 | 0 | 0 |
| 20530 | Slc31a2 | −0.00126407 | −0.00211391 | −0.00414854 | −0.00486515 | −0.00424503 | 0 | 0 |
| 19225 | Ptgs2 | −0.00047187 | 0 | −0.00269762 | −0.00270027 | −0.00423906 | 0 | 0 |
| 74190 | Exoc3l4 | 0.000571527 | −0.00236674 | 0.004418538 | 0.002192926 | 0.004238535 | 0 | −0.0013919 |
| 105387 | Akr1c14 | −0.00137797 | −0.00183743 | −0.0049005 | −0.0049886 | −0.00423778 | 0 | 0 |
| 14282 | Fosb | −0.00026519 | 0 | −0.00304977 | −0.00177147 | −0.00422654 | 0 | 0 |
| 17219 | Mcm6 | 0.000579828 | 0.001809391 | 0.004685652 | 0.003671205 | 0.004224395 | 0 | −0.17380909 |
| 234214 | Sorbs2 | 0.001068317 | 0.000191023 | 0.002791952 | 0.00308338 | 0.004219705 | 0 | 0 |
| 71878 | Fam83d | 0.000914029 | 0 | 0.003178862 | 0.005323313 | 0.004219021 | 0 | 0 |
| 243780 | Dennd11 | −0.00131294 | −0.00282753 | −0.00455447 | −0.00434783 | −0.0042114 | 0 | 0 |
| 56543 | Kcnd3 | 0.001331758 | −0.00013179 | 0.002931736 | 0.002769132 | 0.004209294 | 0 | 0 |
| 12495 | Entpd1 | −0.00113989 | −0.00163333 | −0.00384524 | −0.00390887 | −0.00419836 | 0 | 0 |
| 234728 | Cmtr2 | 0.000773719 | 0.00306716 | 0.005078443 | 0.004761605 | 0.004192307 | 0 | 0 |
| 17250 | Abcc1 | −0.00092189 | −0.00172127 | −0.00373108 | −0.00381133 | −0.00415139 | 0 | 0 |
| 207565 | Camkk2 | −0.00094103 | −0.00328254 | 0.00370513 | −0.00415559 | −0.00414469 | 0 | 0 |
| 72461 | Prcp | −0.00072572 | 2.50E−05 | −0.00066323 | −0.0034451 | −0.00414276 | 0 | 0 |
| 56868 | Psg23 | 0 | −0.00098719 | 0 | 0 | 0 | 0.590306852 | 0 |
| 16597 | Klf12 | 0.00053081 | −0.00154063 | −0.00048963 | 0.000831126 | 0.001842618 | −0.34222315 | −0.0199201 |
| 16202 | Ilk | −0.00155423 | 0.000524882 | 0.000552344 | 7.87E−05 | 0.000816238 | −0.26357168 | 0 |
| 11556 | Adrb3 | −0.00099641 | 0.00112738 | 1.83E−05 | −0.0005617 | −0.00140448 | 0.24611836 | 0.077199047 |
| 227696 | Phyhd1 | −0.00018197 | −0.0015478 | −0.00028244 | −0.00015205 | 0.000116194 | −0.22165354 | 0 |
| 67427 | Rps20 | 0.001761625 | 0.002111343 | 0.002022373 | 0.001515024 | −0.00044148 | −0.16202261 | 0 |
| 19946 | Rpl30 | 0.000410924 | −0.00038817 | 0.000784878 | −0.00026523 | 6.79E−05 | 0.125805004 | 0 |
| 12971 | Crym | −0.00081376 | −0.00359423 | 0.000213032 | −0.00299026 | −0.00021999 | 0.107587973 | 0.201077606 |
| 67942 | Atp5g2 | −0.00178513 | −0.00235846 | −0.00484387 | −0.00433581 | −0.00220808 | −0.08125067 | 0.128771578 |
| 100273 | Osbpl9 | 0.000204887 | −0.00122905 | −0.00145193 | −0.00187396 | −0.00157064 | −0.07368667 | 0 |
| 28084 | Vps25 | −0.00141425 | −0.00046638 | 8.74E−05 | 0.002456034 | 0.000132846 | −0.06536389 | 0 |
| 13537 | Dusp2 | −0.00030756 | −0.00148744 | −0.00162083 | 0.003428996 | −0.0010046 | 0.063053754 | 0 |
| 18212 | Ntrk2 | 0.001609142 | −0.00194265 | −0.00291206 | −0.00267717 | 0.000299099 | 0.039281109 | 0 |
| 67760 | Slc38a2 | −0.0009035 | 0.001059923 | −0.00020646 | −0.00091852 | 0.000194284 | −0.02102616 | 0 |
| 14683 | Gnas | −0.00028342 | −0.00134057 | −0.0003597 | 0.000967458 | −0.00077602 | −0.01332244 | 0 |
| 20515 | Slc20a1 | 0.001642803 | 0.002108408 | 0.000470787 | 0.001583636 | 0.003707872 | −0.00690524 | −0.0001853 |
| 14950 | H13 | 3.12E−05 | −0.00055984 | −0.00193742 | −0.00176379 | −0.00019849 | −0.00537079 | 0 |
| 70466 | Ckap2l | −0.0009191 | 0.003251579 | 0.000478323 | 0.002684241 | −0.00032096 | 0.002151233 | 0 |
| 227522 | Rpp38 | 0.000448914 | 0.00081092 | 0.001104753 | 0.001137837 | 0.00140525 | −0.0019682 | 0 |
| 68939 | Rasl11b | −0.00039679 | −0.00297275 | −0.00267596 | −0.0039554 | −0.00335558 | 0 | 0.367774629 |
| 100039028 | Mup11 | 0 | −0.00110238 | 0 | 0 | 0 | 0 | −0.35887808 |
| 22413 | Wnt2 | −0.00066013 | 0.000473641 | 0.000884813 | −0.0025525 | −0.00140573 | 0 | −0.33933289 |
| 72125 | Amer2 | −8.27E−05 | −0.00227666 | −0.00329424 | −0.00336076 | −0.00120024 | 0 | 0.338835496 |
| 53419 | Corin | 0.000975774 | −0.00129461 | −0.00487444 | −0.0050018 | −0.00236061 | 0 | 0.336280415 |
| 16763 | Lad1 | −0.00067949 | 0.002459605 | −0.00153546 | −0.00231382 | −0.002845 | 0 | −0.33359946 |
| 22234 | Ugcg | 0.000162656 | −0.00066587 | 0.000756692 | 0.001808038 | 0.002521279 | 0 | 0.332617832 |
| 12014 | Bach2 | 0.000532451 | 0.002204563 | 0.002940485 | 0.004141964 | 0.003578715 | 0 | −0.32153514 |
| 17524 | Mpp1 | −0.00098957 | 0.000149081 | −0.00239615 | −0.00329087 | −0.00271731 | 0 | 0.314889453 |
| 14864 | Gstm3 | −0.00098582 | −0.00162261 | −0.00348855 | −0.00507232 | 0 | 0 | 0.292111454 |
| 217335 | Fbf1 | −0.00141098 | −0.00221779 | −0.00101081 | −0.00086263 | −0.00086222 | 0 | 0.291753978 |
| 625018 | C4a | 0 | −0.00152832 | 0 | 0 | 0 | 0 | 0.281124077 |
| 406220 | Krt77 | 0.000108274 | 0.00365837 | 0.003349578 | 0.00211097 | 0.001592585 | 0 | −0.27818984 |
| 78252 | Nxpe2 | −0.00138435 | −0.00258694 | −0.00206671 | −0.00160729 | −0.00136225 | 0 | 0.275102651 |
| 12686 | Elovl3 | −0.00016747 | −0.0025735 | −0.00333124 | −0.00401255 | −0.00277821 | 0 | 0.271719114 |
| 20219 | Apcs | 0.001531402 | −0.00313944 | −0.00433906 | −0.00500392 | −0.00079761 | 0 | −0.26123778 |
| 225994 | Nmwrk1 | −1.44E−05 | −0.00267216 | −0.00104375 | −0.00087057 | 0.000226446 | 0 | 0.251783385 |
| 56338 | Txnip | 0.000475027 | −0.00183331 | −0.0043976 | −0.00388722 | 0.000148663 | 0 | 0.250736609 |
| 17064 | Cd93 | 0.00010968 | 0.001578268 | 1.19E−05 | 0.001545005 | −3.86E−05 | 0 | −0.24872292 |
| 240549 | Gm4952 | 0.00054424 | −0.00379754 | −0.00463129 | −0.00370326 | 0 | 0 | 0.238547711 |
| 71898 | Apol9b | 0 | 0.002286119 | 0.001316592 | 0 | 0 | 0 | −0.23786238 |
| 100041658 | Mup7 | 0 | 0.00012135 | 0 | 0 | 0 | 0 | −0.23773819 |
| 12144 | Blm | 0.000564132 | 0.003415344 | 0.00137614 | 0.001468407 | 0.00140967 | 0 | −0.2338487 |
| 18024 | Nfe2l2 | 0.001288816 | 0.000346538 | 0.003220474 | 0.003523483 | 0.002617161 | 0 | 0.233118659 |
| 16592 | Fabp5 | −0.00039995 | −0.00032941 | −0.0013026 | −0.00111583 | −0.00196017 | 0 | 0.23076041 |
| 228413 | Prrg4 | 0.001028335 | 0.002132044 | 0.00400856 | 0.004259122 | 0.002704257 | 0 | −0.2300887 |
| 320782 | Tmem154 | 0.001138723 | −0.00381164 | −0.00253375 | −0.00326533 | −0.00133086 | 0 | 0.226914614 |
| 72776 | Sass6 | −0.00101073 | −0.00196145 | −0.00035279 | 0.000734108 | −0.00015317 | 0 | 0.222804716 |
| 216227 | Slc1728 | 0 | 0.001077117 | 0.000551137 | 0 | 0 | 0 | 0.221933687 |
| 21807 | Tsc22d1 | 0.00037565 | 0.003369898 | 0.000675995 | −0.00048907 | 0.001090475 | 0 | −0.22050051 |
| 12577 | Cdkn1c | 0.00047869 | −0.00035119 | −0.00036268 | −3.73E−05 | 0.001367664 | 0 | 0.210175482 |
| 19200 | Pstpip1 | −0.00025991 | −0.00135859 | −0.00065185 | −0.0034102 | −0.00028053 | 0 | 0.207552174 |
| 71687 | Tmem25 | 0.00060451 | −0.00032217 | 0.002287146 | 0.000497133 | 0.002115056 | 0 | 0.207125343 |
| 239122 | Setdb2 | 0.000957647 | 0.00191609 | 0.000937709 | 0.002360144 | 0.001827831 | 0 | −0.20281776 |
| 72948 | Tppp | −0.00054034 | 0.002239449 | 0.000826758 | −0.00075234 | −0.00290871 | 0 | −0.20201396 |
| 21676 | Tead1 | 1.52E−05 | −0.00059155 | −0.00122394 | −0.00206006 | −0.0022132 | 0 | −0.19829305 |
| 245522 | Zc4h2 | −5.40E−05 | −0.00038499 | −0.00092792 | 7.65E−05 | −0.0009872 | 0 | −0.19804724 |
| 379043 | Raet1e | 0 | −0.00196552 | 0 | 0 | 0 | 0 | 0.197552816 |
| 68481 | Mpzl1 | −0.00088566 | −0.00057745 | −0.0015273 | −0.00312792 | −4.66E−05 | 0 | −0.19596698 |
| 22414 | Wnt2b | 1.59E−05 | −0.00086683 | −0.00041218 | 0.002495225 | −4.74E−05 | 0 | 0.190023635 |
| 77037 | Mrap | −0.00092513 | −0.00103213 | −0.00085781 | −0.00322855 | −0.0009266 | 0 | 0.189636705 |
| 117198 | Ivns1abp | −0.00161944 | −0.00175265 | −0.00147629 | −0.00299793 | −0.00197732 | 0 | 0.188525229 |
| 70122 | Mllt3 | 0.001125656 | 0.00093516 | 0.003295877 | 0.0026448 | 0.002572494 | 0 | −0.18702508 |
| 404195 | Cyp2c54 | 0 | 0.001813045 | 0 | 0 | 0 | 0 | 0.186991293 |
| 211924 | Dsg1c | 0 | 0.001975179 | 0.001066896 | 0 | 0 | 0 | −0.18680043 |
| 217843 | Unc79 | −0.00023095 | 0.002665645 | 0.002485755 | 0.002330119 | 0.002742441 | 0 | 0.184466614 |
| 223881 | Rnd1 | 0.000914911 | −0.00146487 | 0.001149584 | 0.000272034 | −0.00053824 | 0 | −0.18438617 |
| 74521 | Ppp4r4 | 0.001132679 | −0.00020568 | 0.001504369 | 0.002297992 | 0.001813053 | 0 | −0.18417174 |
| 67887 | Saraf | 0.000193443 | 0.002286659 | 0.002428 | 0.001520584 | 0.002655331 | 0 | −0.18354396 |
| 67846 | Tmem39a | −0.00026597 | −0.00163794 | −0.00098559 | −0.00025095 | −0.0003551 | 0 | 0.18251927 |
| 53791 | TlrS | −0.00128458 | −1.82E−05 | −0.0035709 | −0.00172874 | −0.00107195 | 0 | −0.18120221 |
| 639769 | Adh6-ps1 | 0 | −0.00179101 | 0 | 0 | 0 | 0 | 0.177265895 |
| 212518 | Sprn | 0.000336877 | 0.00203728 | 0.004801319 | 0.004955026 | 0.001362417 | 0 | −0.17155346 |
| 16415 | Itgb3 | 0.000720463 | −0.0007005 | −7.94E−05 | 0.001131834 | 0.002937193 | 0 | −0.16873038 |
| 12660 | Chka | 0.000753172 | −0.00051522 | 0.000421591 | −3.71E−05 | 0.00048115 | 0 | −0.16802752 |
| 68861 | Dipk2a | −0.00161813 | −0.00326912 | −0.00386405 | −0.00382665 | −0.00225458 | 0 | 0.167917598 |
| 434794 | Xlr4a | 0 | −0.00240411 | 0 | 0 | 0 | 0 | 0.167607025 |
| 21952 | Tnni1 | 0.000344852 | −0.00210747 | −0.00033748 | 0.000218712 | 0.00025623 | 0 | 0.16672948 |
| 236643 | Sytl5 | 0.000113329 | 0.001224257 | −0.00235417 | −0.00358815 | −0.00200154 | 0 | −0.16567647 |
| 52040 | Ppp1r10 | −0.00085136 | 0.001025983 | 0.000171062 | −0.0005795 | −0.00023062 | 0 | 0.164273238 |
| 68955 | Srrm4 | −0.00021707 | −0.00286789 | −0.00388864 | −0.00532948 | −0.00267445 | 0 | 0.163064865 |
| 21824 | Thbd | 0.000599261 | 0.002207308 | −0.0009142 | −0.0009414 | 0.000158367 | 0 | −0.16126075 |
| 268741 | Tox4 | −0.00056067 | 0.000888347 | −0.00204532 | −0.00176146 | −0.00131571 | 0 | −0.16082559 |
| 109222 | Rarres1 | −0.00100379 | −0.00181918 | −0.00481626 | −0.00332802 | −0.00178687 | 0 | 0.158497473 |
| 100503019 | Gm16551 | 0 | 0.001890332 | 0 | 0 | 0 | 0 | 0.15546135 |
| 30927 | Snai3 | −0.0008314 | −0.0033611 | −0.00037215 | −0.0003535 | −0.00149501 | 0 | 0.154871486 |
| 56273 | Pex14 | 0.000467102 | 0.000696764 | 0.000761885 | 0.00016261 | −0.0001787 | 0 | −0.15253371 |
| 99382 | Abtb2 | 0.001134417 | 0.00154262 | 0.003013406 | 0.003682574 | 0.000975681 | 0 | −0.15181788 |
| 110751 | Adam33 | −0.00084219 | −0.00098756 | −0.00220581 | −0.00332798 | −0.00382985 | 0 | 0.151813206 |
| 170744 | Tlr8 | 0.000844598 | 0.000884652 | 0.002443951 | −0.00051249 | −3.33E−05 | 0 | 0.150355316 |
| 18516 | Pbx3 | 0.000889511 | −0.00325952 | 0.00045798 | 0.002733235 | 0.002775173 | 0 | 0.148044629 |
| 229658 | Vangl1 | −0.00145947 | −0.00050107 | 0.00077729 | 0.000858279 | −0.00142757 | 0 | −0.14776962 |
| 20209 | Saa2 | 0.001406164 | −0.00349447 | −0.00098603 | −0.00049089 | 0 | 0 | 0.146641761 |
| 213236 | Dnd1 | −0.00048629 | −0.00274436 | 0.000892062 | −0.0003499 | −0.00028434 | 0 | 0.146337424 |
| 67533 | Ppfibp1 | −0.00040887 | 0.002549822 | 0.002804858 | 0.001852909 | 0.001260619 | 0 | −0.14530698 |
| 11484 | Aspa | 0.000634129 | −0.00214987 | −0.00139206 | −0.00499186 | −0.00184098 | 0 | 0.145149596 |
| 665033 | Col6a5 | −0.00042987 | 0.000104037 | 0.00060888 | −0.00137936 | −0.00204921 | 0 | 0.144159938 |
| 228421 | Kif18a | −0.00034352 | 0.000588371 | 0.000790241 | −0.00021974 | 0.000787038 | 0 | −0.1439888 |
| 100038948 | Mup9 | 0 | −0.00141667 | 0 | 0 | 0 | 0 | −0.14370505 |
| 244416 | Ppp1r3b | 0.001133443 | 0.002170296 | 0.002633418 | 0.002593395 | 0.000809836 | 0 | −0.14364545 |
| 107094 | Rrp12 | −0.00041977 | 0.000424758 | −0.00166174 | −0.00444103 | −0.00308991 | 0 | 0.142562068 |
| 17991 | Ndufa2 | −0.00111182 | 0.000543688 | −0.00108053 | −0.00182929 | −0.00153261 | 0 | 0.141577856 |
| 17167 | Marco | −0.00198194 | 0.002064217 | −0.0013275 | −0.00098388 | −0.00175603 | 0 | −0.14154172 |
| 22340 | Vegfb | 0.001339091 | −6.54E−05 | 0.000479033 | 0.001324794 | 0.001400278 | 0 | 0.139542213 |
| 15959 | Ifit3 | 0.000217714 | 0.003604667 | 0.000901108 | 0.000988661 | 0.000836827 | 0 | 0.139363939 |
| 224481 | Tfb1m | 0.000815473 | 0.002366805 | 0.00267948 | 0.001660775 | 0.002072235 | 0 | −0.13855348 |
| 18750 | Prkca | −0.0004744 | −0.00138639 | −0.00206395 | −0.00194899 | −0.00038119 | 0 | −0.13797614 |
| 217316 | Slc16a5 | −0.00134155 | 0.00245696 | −0.00157564 | −0.00095823 | −0.00159106 | 0 | −0.1364869 |
| 54124 | Cks1b | −0.00014514 | −0.00133069 | −0.0028663 | −0.00339164 | −0.00109843 | 0 | 0.133795141 |
| 56321 | Aatf | 0.001551011 | 0.002465352 | 0.003759553 | 0.004610391 | 0.002853789 | 0 | −0.13376692 |
| 320204 | Etfbkmt | −0.00102629 | −0.00293567 | −0.00193499 | −0.00367985 | −0.0020138 | 0 | 0.133009275 |
| 69083 | Sult1c2 | 0.000791066 | 0.002867641 | 0.002726563 | 0.002681538 | 0.001407369 | 0 | −0.13292273 |
| 15903 | Id3 | 0.001339742 | −0.00187856 | −0.00089023 | 0.000434489 | 0.0014204 | 0 | 0.132895968 |
| 22042 | Tfrc | −0.00035554 | 0.00126032 | 0.001564798 | −0.00221028 | −0.00158993 | 0 | −0.13257595 |
| 72056 | 1810055G02Rik | −0.00034014 | 0.001055587 | 0.002400459 | 0.002448085 | 0.002297552 | 0 | −0.13216288 |
| 55936 | Ctps2 | 0.000976801 | 0.001305346 | 0.000796093 | 0.000454102 | 0.000148965 | 0 | −0.13203202 |
| 80837 | Rhoj | −8.26E−05 | −0.00248861 | −0.00166574 | −0.00241118 | −0.00234845 | 0 | −0.1312938 |
| 13712 | Elk1 | −0.00057602 | 0.000184983 | −0.00031196 | −0.00061748 | −2.00E−05 | 0 | −0.13099182 |
| 13897 | Ces1e | 2.68E−05 | 0.003105706 | 0.002117544 | 0.00110237 | 0 | 0 | −0.13096739 |
| 12489 | Cd33 | −0.00038538 | 0.001164421 | 0.000510421 | 0.002076159 | 0.001759423 | 0 | −0.13069606 |
| 19265 | Ptprcap | 2.07E−05 | 0.000204049 | −0.00378466 | −0.00474787 | −0.00194763 | 0 | 0.130180382 |
| 17254 | Slc3a2 | 0.000634774 | −0.00027577 | 0.003543054 | 0.003756818 | 0.002784386 | 0 | −0.12960665 |
| 76792 | Spring1 | 0.000547314 | 0.002903763 | 0.002103377 | 0.00048538 | 0.000489837 | 0 | −0.12950825 |
| 19193 | Pipox | −0.00027792 | −0.00057921 | −0.00069905 | −0.00310383 | −0.00297373 | 0 | 0.128125926 |
| 55947 | Ddre1a | −0.00071762 | 0.001296207 | −0.00264476 | −0.0020261 | −0.00114028 | 0 | 0.127047683 |
| 71712 | Dram1 | −0.0001409 | −5.58E−05 | −9.18E−05 | −0.00074785 | −0.00042943 | 0 | 0.126454574 |
| 11639 | Ak4 | 0.001294095 | 0.000675723 | 0.002631308 | 0.001491638 | 0.00070022 | 0 | 0.126427745 |
| 67776 | Vwa5a | 0.001346864 | 0.002762746 | 0.003586177 | 0.003609827 | 0.001576927 | 0 | −0.12627946 |
| 234463 | Tmem184c | −0.00044745 | 0.001484187 | 0.002088513 | 0.001447117 | 0.000181137 | 0 | −0.12616895 |
| 224019 | Tmem191c | 0.00045092 | −0.00170876 | −0.00103889 | −0.00128789 | 0 | 0 | 0.125634699 |
| 242705 | E2f2 | 0.0012603 | 0.002917874 | 0.001222638 | 0.00107055 | 0.002823139 | 0 | −0.12470028 |
| 100213 | Rusc2 | 5.19E−05 | −0.0018781 | −0.00054961 | −0.00101968 | −0.0008852 | 0 | 0.12458877 |
| 245026 | Col6a6 | −0.00165782 | −0.00086728 | −0.00122064 | −0.00035385 | −0.00119064 | 0 | 0.124570279 |
| 226591 | Tiprl | 0.000254633 | 0.001324648 | 0.000347786 | 0.001211771 | −0.00057058 | 0 | −0.12413899 |
| 18441 | P2ry1 | −0.00128721 | 0.000261188 | −0.00295866 | −0.00321301 | −0.00121227 | 0 | 0.123982726 |
| 74430 | Cfap20dc | −0.00103401 | −0.002369 | −0.00292536 | −0.00298024 | −0.00133396 | 0 | 0.123744381 |
| 71733 | Susd2 | 0.000984494 | 0.002550844 | −0.00226773 | −0.0015563 | −0.00015999 | 0 | 0.123126106 |
| 235431 | Coro2b | −0.00021749 | 0.001086701 | −8.26E−05 | 0.000999595 | 0.000845172 | 0 | −0.12295247 |
| 106947 | Slc39a3 | −0.00108543 | 0.00125955 | −0.00102055 | −0.00151164 | −0.00092661 | 0 | −0.12275468 |
| 22687 | Zpr1 | 0.00115043 | 0.001170089 | 0.000298251 | 0.001386715 | 0.000152321 | 0 | 0.122506864 |
| 338467 | Morc3 | 0.000746088 | 0.001597795 | −0.00064314 | 0.000495843 | −0.00017416 | 0 | −0.12147 |
| 449521 | Zfp213 | −0.00035259 | −0.00093754 | −0.00184513 | −0.00362336 | −0.00024634 | 0 | 0.121037875 |
| 252838 | Tox | 0.000200568 | −0.00078228 | −0.00042608 | 0.002249964 | 0.001225086 | 0 | −0.12078613 |
| 67492 | Zfand4 | 0.00202382 | 0.001862693 | 0.003722645 | 0.00422265 | 0.00379707 | 0 | 0.120760702 |
| 74088 | Them7 | −7.29E−05 | 0.001709238 | 0.001366007 | 0.002227757 | 0 | 0 | 0.120157015 |
| 69123 | Eci3 | −0.00134824 | −0.00186992 | −0.00211446 | −0.00458757 | 0 | 0 | 0.120060981 |
| 432613 | Trim80 | −0.000273 | −0.00219042 | −0.00276136 | −0.00409586 | 0 | 0 | 0.119462843 |
| 17295 | Met | −0.00063563 | −0.00135702 | −0.00298671 | −0.0036071 | −0.00189526 | 0 | 0.118796067 |
| 234671 | Ces2c | 0 | −0.00167264 | 0 | 0 | 0 | 0 | 0.117857486 |
| 170822 | Usp33 | −0.00053308 | 0.002269399 | 0.0007953 | 0.000336719 | 0.000703318 | 0 | −0.11782814 |
| 73332 | Ccdc30 | −0.00046203 | 0.001210701 | 0.000121622 | 0.000625171 | −7.28E−05 | 0 | −0.1174642 |
| 207215 | Fbxo40 | −0.00097113 | 0.003196758 | −0.00192655 | −0.00401307 | −0.00089098 | 0 | −0.11707319 |
| 73205 | C9orf72 | −0.00018346 | −0.00049693 | −0.00064966 | 3.96E−05 | −0.00044022 | 0 | −0.11705733 |
| 67609 | 4930453N24Rik | −6.57E−05 | −0.0010401 | 0.000990709 | 0.001970737 | 0.000756071 | 0 | −0.11580401 |
| 259301 | Leap2 | 0 | 0.001709195 | 0.004297726 | 0 | 0.002996758 | 0 | −0.11490956 |
| 67460 | Decr1 | 0.001111163 | 5.07E−06 | −0.00111124 | 0.001378166 | −0.00087506 | 0 | 0.114863431 |
| 28250 | Slco1e4 | −0.00016118 | −0.00094832 | −0.00217678 | −0.00316549 | −0.00247938 | 0 | −0.11426188 |
| 80885 | Hcar2 | −0.00080693 | 0.00286078 | 0.003506182 | 0.004864692 | 0 | 0 | −0.11351598 |
| 97884 | B3galnt2 | −0.00085653 | 0.001950628 | 0.000375766 | 0.000297429 | −0.00021282 | 0 | −0.11312542 |
| 21346 | Tagln2 | 0.000296476 | 0.000590421 | 0.00220661 | 0.001893252 | 0.001242407 | 0 | −0.11220826 |
| 13682 | Eif4a2 | −0.00086948 | 0.001271295 | −0.00137814 | −0.00098266 | −0.00075158 | 0 | −0.11183576 |
| 12765 | Cxcr3 | 0.000479107 | −0.00011091 | 0.001145018 | 0.003924626 | 0.00103937 | 0 | 0.111592658 |
| 77579 | Myh10 | −0.0004871 | −0.0035246 | 0.002391981 | 0.001192271 | 0.000144758 | 0 | −0.11139789 |
| 11676 | Aldoc | 0.001037023 | 0.000617831 | 0.000678415 | −0.00110154 | 0.001956048 | 0 | 0.11107796 |
| 71492 | Bbs7 | −0.00063744 | 0.001811921 | −0.00121898 | −0.00102883 | −0.00122325 | 0 | −0.11094421 |
| 224170 | Dzip3 | −0.00174566 | −0.00179861 | −0.00362871 | −0.0050585 | −0.00192322 | 0 | 0.110920425 |
| 378937 | Lrrc24 | −1.25E−05 | 0.002394253 | 0.00048887 | 0.000373169 | 0.000700054 | 0 | −0.11081034 |
| 19707 | Reps1 | 0.000464905 | 0.000725299 | 0.001598746 | 0.002284052 | 0.000799948 | 0 | −0.11047522 |
| 319530 | Zfp750 | −0.00089314 | −0.00226643 | −0.00243514 | −0.00251883 | −0.00024428 | 0 | 0.110162976 |
| 14218 | Sh3pxd2a | 0.000461339 | 0.000406592 | 9.52E−05 | 7.89E−07 | 0.000434299 | 0 | −0.11008643 |
| 66255 | Hsbp1l1 | −0.00021462 | 0.00151245 | 0.002743966 | 0.003702134 | 0.002295977 | 0 | −0.10919571 |
| 69430 | 1700048O20Rik | 0 | 0.001010573 | 0 | 0 | 0 | 0 | −0.10891404 |
| 109323 | C1qtnf7 | −0.00025094 | 0.000180975 | 0.001925063 | 0.001464408 | 0.00190838 | 0 | −0.1079343 |
| 15331 | Hmgn2 | −0.00102358 | 0.00232293 | −0.00127809 | −0.00145135 | −0.00133234 | 0 | −0.10661679 |
| 74309 | Osbp2 | −0.00016841 | −0.00245027 | −0.004356 | −0.003298 | −0.00138229 | 0 | 0.106578965 |
| 19737 | Rgs5 | 0.001369496 | −0.00102658 | −0.00210845 | −0.0021151 | 0.00040932 | 0 | −0.10635996 |
| 12401 | Serpina6 | 2.41E−05 | −0.00203628 | 0.000791019 | −0.00025021 | 0.000936073 | 0 | 0.10610149 |
| 78779 | Spata2l | −0.00075073 | −0.00107005 | 0.0029333 | 0.002466262 | −0.00089322 | 0 | −0.10551115 |
| 17472 | Gbp4 | 0.000697967 | −0.00112713 | 0.001426614 | 0.004749165 | 0.002805426 | 0 | 0.104961337 |
| 381199 | Tmem151a | −0.00075188 | −0.00049938 | −0.004104 | −0.00393587 | −0.00392851 | 0 | 0.104905293 |
| 24052 | Sgcd | 0.00043128 | 0.000765937 | −0.00123426 | −0.00170881 | −0.00013011 | 0 | −0.10346782 |
| 231201 | Semp2l2a | 0 | −0.00032292 | 0 | 0 | 0 | 0 | 0.103319945 |
| 76633 | Lrmda | 0.000356404 | 0.001469311 | 0.002311291 | 0.001606158 | 0.00268051 | 0 | −0.1029521 |
| 12467 | Cct6b | 8.32E−05 | −0.00136223 | −0.00092986 | 0.000262542 | −0.00108385 | 0 | −0.1022567 |
| 104871 | Spata7 | −9.05E−05 | −0.00104142 | −0.00173766 | −0.0006822 | −0.00139941 | 0 | −0.10219466 |
| 319173 | H2ac10 | 0 | −0.00199005 | 0 | 0 | 0 | 0 | −0.10205521 |
| 353282 | Sfmbt2 | −0.00106774 | 0.001633159 | −0.00201699 | −0.0021639 | −0.00099205 | 0 | −0.1020036 |
| 216134 | Pdxk | −2.99E−05 | −0.00184053 | −0.00261676 | −0.00314657 | −0.00220093 | 0 | 0.101937512 |
| 319942 | A530016L24Rik | 1.20E−05 | −0.00067048 | −0.00130651 | −0.00128182 | −0.00052377 | 0 | 0.100681045 |
| 15982 | Ifrd1 | 0.000565533 | 0.000804737 | 0.001762321 | 0.002191635 | 0.001564579 | 0 | −0.10021313 |
| 54139 | Irf6 | 0.00054053 | 0.002153381 | −0.00061663 | −0.00205705 | −0.00038015 | 0 | −0.10019706 |
| 13838 | Epha4 | −0.00111448 | 3.71E−05 | −0.00179053 | −0.00083029 | −0.00099261 | 0 | −0.10014825 |
| 99929 | Tiparp | 0.001338499 | 0.002302893 | 0.001054825 | 0.001490782 | 0.000301533 | 0 | −0.09981331 |
| 73149 | Clec4a3 | 0.000499734 | −0.00368036 | −0.00123924 | 0.00037557 | 0.001416041 | 0 | 0.098924935 |
| 16924 | Lnx1 | 6.51E−05 | 0.003759958 | 0.001395904 | 0.00320447 | 0.000632878 | 0 | −0.09865627 |
| 100039054 | Mup12 | 0 | 0.00069967 | 0 | 0 | 0 | 0 | −0.09833351 |
| 207181 | Rbms3 | −0.00156268 | 0.00082758 | −0.00309788 | −0.00285701 | −0.00280508 | 0 | −0.09818229 |
| 70240 | Ufsp1 | 0.000410461 | −0.00197678 | −0.00046264 | −0.00041192 | 0.001720412 | 0 | 0.097873886 |
| 78558 | Htra3 | −0.00050505 | 6.60E−05 | −0.00069471 | −0.00092105 | −0.00189093 | 0 | 0.097832585 |
| 22590 | Xpa | 0.00077754 | 0.00119739 | −0.00138491 | −0.0016626 | −0.0001556 | 0 | 0.097353186 |
| 75345 | Slamf7 | 0.001321126 | −0.00349353 | 0.00132531 | −0.00011927 | 0.001940077 | 0 | 0.096621639 |
| 218476 | Gcnt4 | 0 | 0.001028271 | 0 | 0 | 0 | 0 | −0.09659172 |
| 18763 | Odc1 | −0.00065052 | −0.00053376 | 0.000343093 | 0.00250143 | 0.000906957 | 0 | −0.09634128 |
| 192232 | Hps4 | −0.00118873 | −0.00086303 | 0.000298403 | −0.00108252 | −0.0010103 | 0 | −0.09617148 |
| 66309 | Tmem128 | 0.000276998 | 0.002026158 | 0.001421045 | 0.001059648 | 0.001435721 | 0 | −0.09583526 |
| 14870 | Gstp1 | −0.0003162 | 0.001308835 | 0.000813606 | 0.000480724 | 0.000739765 | 0 | 0.095648296 |
| 18674 | Slc25a3 | 0.000147679 | −0.00013911 | 0.001242164 | 0.001067991 | 0.001047315 | 0 | −0.09534218 |
| 232341 | Wnk1 | 0.000662744 | 0.001699726 | −0.00085108 | −0.00057721 | 0.000348047 | 0 | −0.09488819 |
| 18585 | Pde9a | 0.00022187 | 0.002687997 | 0.001955638 | 0.001383275 | 0.00059701 | 0 | −0.09407611 |
| 14263 | Fmo5 | 0.001340107 | −0.0002364 | −0.00122805 | 0.002558961 | 0.001055035 | 0 | 0.094040675 |
| 14726 | Pdpn | 0.001742358 | −0.00142247 | 0.00158868 | 0.004130701 | 0.003502627 | 0 | 0.093771758 |
| 381549 | Zfp69 | −0.00016254 | 0.001905465 | 0.004177831 | 0.004119077 | 0.001704251 | 0 | −0.09348036 |
| 27359 | Sytl4 | 0.000300136 | 0.001300955 | −0.00029836 | 0.002930818 | 0.000833028 | 0 | −0.09345608 |
| 216856 | Nlgn2 | 0.000766628 | 0.000701915 | 0.002070615 | 0.004285003 | 0.002802654 | 0 | 0.093367367 |
| 74023 | Rd3 | 0.000531644 | 0.002106821 | 0.000802728 | 0.001367257 | 0.000980479 | 0 | −0.09305428 |
| 16430 | Stt3a | 0.00013968 | 0.0027264 | 0.000813393 | 0.000752569 | 0.001813683 | 0 | −0.09283479 |
| 320747 | Lingo4 | 1.61E−05 | −0.00332065 | −0.0040375 | −0.00193958 | −0.00090831 | 0 | 0.092175936 |
| 20516 | Slc20a2 | 0.00060393 | 0.000415352 | 0.002092215 | 0.001976239 | 0.001770552 | 0 | −0.09182353 |
| 74153 | Uba7 | 0.001210396 | −0.00269451 | 0.000823884 | 0.001139945 | 0.000582595 | 0 | −0.09172457 |
| 79221 | Hdac9 | −0.00041702 | 0.000683218 | 0.000645823 | 0.002631416 | 0.001041101 | 0 | −0.09170924 |
| 20522 | Slc23a1 | −8.88E−05 | 0.00070301 | 0.003031547 | 0.001408848 | 0.002064339 | 0 | −0.09150443 |
| 19299 | Abcd3 | −0.00093775 | −0.00090062 | −0.00059794 | −0.00191279 | −0.00122533 | 0 | 0.090953586 |
| 66939 | Aagab | 0.000462192 | 0.001012717 | −0.00018179 | 0.000259496 | 0.000362753 | 0 | −0.09033655 |
| 66153 | Fbxo36 | −0.00063284 | −0.00251948 | −0.00200935 | −0.00290571 | −0.00207238 | 0 | 0.090130165 |
| 11554 | Adrb1 | −0.0003719 | −0.00154668 | 0.003160583 | 0.003576347 | 0.00124168 | 0 | 0.090093762 |
| 223753 | Cerk | 0.00036851 | 0.003036929 | 0.000707537 | 2.27E−05 | 0.000601142 | 0 | −0.08994873 |
| 170768 | Pfkfb3 | −0.00032963 | −0.00032254 | −0.00135165 | −0.00278341 | −0.00296547 | 0 | 0.089613692 |
| 53978 | Lpar2 | 0.001244217 | 0.003342109 | 0.003137214 | 0.002806271 | 0.003277584 | 0 | −0.08955847 |
| 57784 | Bin3 | 0.001684947 | 0.002004294 | 0.003931982 | 0.002628197 | 0.002251544 | 0 | −0.08942613 |
| 107022 | Gramd3 | −0.00158641 | −0.00155119 | −0.00185336 | −0.00187636 | −0.00072984 | 0 | 0.087880626 |
| 107373 | Fam111a | −0.00055459 | 0.001292865 | 0.000909776 | 0.002047435 | 0.000278278 | 0 | −0.08780008 |
| 12409 | Cbr2 | −0.00011467 | −0.00102944 | 0.004347031 | 0.003608193 | 0 | 0 | 0.087718105 |
| 19823 | Rnf7 | −0.00087014 | −0.00030405 | 0.000116809 | −0.0014726 | −0.00075384 | 0 | −0.08763602 |
| 77832 | Tchp | 4.56E−05 | −0.00188826 | −4.50E−05 | −0.00113505 | −0.00042578 | 0 | 0.087388387 |
| 228839 | Tgif2 | −0.00057912 | −0.00066971 | 0.001318387 | 0.000631635 | −0.00066154 | 0 | 0.087270833 |
| 15109 | Hal | 0.000853853 | −0.00097428 | 0.001904744 | 0.000265536 | 0.000275613 | 0 | 0.08723769 |
| 56699 | Cdc42ep4 | −0.00073247 | −0.00254505 | −0.00331277 | −0.00395094 | −0.00243929 | 0 | 0.08623719 |
| 338362 | Ust | −0.00083392 | 0.000959015 | 0.000125217 | 0.001579277 | −0.00330892 | 0 | −0.08556946 |
| 67304 | 3110070M22Rik | 0 | 0.002484679 | 0 | 0 | 0 | 0 | −0.0848314 |
| 266645 | Acmsd | −0.00027434 | 0.001644239 | −0.0007889 | −0.00073898 | −0.00022374 | 0 | −0.0846922 |
| 68279 | Mcoln2 | 0.001320325 | −0.00371279 | −0.00290024 | −0.00529845 | −0.00379293 | 0 | 0.084419808 |
| 66377 | Ndufc1 | 0.000175215 | −0.00029435 | −0.00032055 | −0.00048322 | −0.00021577 | 0 | 0.084180599 |
| 103136 | Pwp1 | 0.000575269 | 0.001900814 | 0.001964432 | 0.002395008 | 0.001381468 | 0 | −0.08415549 |
| 242642 | Hpdl | −0.00107318 | −0.00195456 | −0.00171578 | −0.0017442 | −0.00197615 | 0 | 0.083888676 |
| 16373 | Irx3 | 0.000744043 | 0.000546532 | 0.002030328 | 0.001155393 | 0.001615585 | 0 | −0.08379904 |
| 212398 | Frat2 | −0.00156435 | 0.00061389 | 0.00138677 | 0.003511966 | −0.00056413 | 0 | 0.083791711 |
| 74157 | Cmtr1 | 0.001090819 | 0.000260691 | −0.00101844 | −0.00042362 | −0.00040691 | 0 | −0.08357524 |
| 18578 | Pde4b | 0.000263281 | −0.00062842 | −5.71E−05 | −0.00081212 | 0.00035147 | 0 | −0.08332131 |
| 12223 | Btc | 0.00028899 | −0.00241837 | −0.00332885 | −0.00362136 | −0.00123323 | 0 | −0.08326313 |
| 57764 | Ntn4 | 0.001590088 | 0.000776881 | 0.001946168 | 0.000887499 | 0.000955545 | 0 | −0.08305778 |
| 14858 | Gsta2 | 0 | −0.0005035 | 0.005011772 | 0 | 0 | 0 | 0.082854169 |
| 245049 | Myrip | 0.001431918 | −0.00137076 | −0.00015577 | 0.0020128 | 0.002133394 | 0 | 0.082766479 |
| 382066 | Prdm10 | 7.68E−05 | −0.00248386 | −0.00021174 | −0.00106562 | −7.48E−05 | 0 | 0.082444256 |
| 76686 | Clip3 | −0.00046307 | 0.000341634 | −0.00088895 | −0.00082648 | −0.00186076 | 0 | 0.082209958 |
| 226407 | Rab3gap1 | −6.80E−05 | 0.000711169 | 0.000640738 | 0.001506634 | 0.00093894 | 0 | −0.08199181 |
| 67307 | Pbld2 | −0.00011605 | 0.003225301 | 0.001720687 | 0.002546968 | 0 | 0 | −0.08193759 |
| 100039139 | Ccdc152 | −0.0007229 | −0.00371142 | −0.00402628 | −0.00412216 | −0.00341478 | 0 | 0.081760416 |
| 20621 | Snn | 0.000333346 | 0.000368928 | 0.002422983 | 0.002906417 | 0.001047925 | 0 | −0.08131225 |
| 83382 | Siglece | −0.00035783 | −0.00143046 | −0.0019654 | 0.001595871 | −0.00014201 | 0 | 0.081307898 |
| 16428 | Itk | −0.0001459 | 0.000170966 | −0.0047098 | −0.00409417 | −0.0032797 | 0 | 0.081288842 |
| 238803 | Zfp366 | −0.00074127 | −0.00180317 | 0.000211305 | 0.00023449 | 0.000221585 | 0 | 0.080963558 |
| 224598 | Zfp758 | −0.00064392 | −3.17E−05 | −0.00394877 | −0.00397343 | −0.00161393 | 0 | 0.080936298 |
| 74413 | Tc2n | 0.00011378 | −0.00084903 | 0.004736157 | 0.00326832 | 0.001874456 | 0 | −0.08066991 |
| 70315 | Hdac8 | −0.001112 | −0.00031938 | −0.00231005 | −0.00224038 | −0.00142963 | 0 | −0.08036284 |
| 68014 | Zwilch | −0.00019239 | 9.18E−05 | 0.001086028 | 0.00249718 | 0.001734223 | 0 | 0.08003408 |
| 67487 | Dhx40 | −0.00103248 | 0.000653947 | −0.00255611 | −0.00252448 | −0.00169254 | 0 | −0.07997928 |
| 22068 | Trpc6 | −0.00081547 | −0.00165116 | 0.00011358 | 0.001205921 | −0.00033386 | 0 | 0.079029797 |
| 20504 | Slc17a1 | 3.51E−06 | 0.002438919 | 0.001926507 | 0.002006131 | 0.001099865 | 0 | −0.07863016 |
| 71145 | Scara5 | −2.22E−06 | −0.00389463 | −0.00165026 | 0.001886399 | −0.00167871 | 0 | 0.078501791 |
| 74008 | Arsg | 0.001389962 | 0.00066221 | 0.002089455 | 0.002964763 | 0.003431041 | 0 | −0.07809781 |
| 102294 | Cyp4v3 | 0.000320464 | 0.000393872 | −0.00024202 | −0.00248864 | −0.00096681 | 0 | −0.07737622 |
| 67171 | Dram2 | −0.0001157 | 0.000101214 | −0.00192554 | −0.0012309 | −0.00148355 | 0 | 0.077034701 |
| 20682 | Sox9 | 0.000736378 | 0.001340255 | −0.00096108 | −0.00047968 | 0.000831073 | 0 | 0.07656082 |

### Examples

The invention is further described in the following examples, which do not limit the scope of the invention described in the claims.

### Materials and Methods

The following materials and methods were used in the Examples below.

### Preprocessing of Gene Expression Training Data

For ITP data, liver samples from compound-treated male and females mice along with age- and sex-matched corresponding controls were collected (Table S1). RNA was extracted from tissues with PureLink RNA Mini Kit as described in the protocol and passed to paired-end 150 bp sequencing on the Illumina NovaSeq 6000. Reads were aligned to Mus musculus genome (GRCm38) using STAR (version 2.5.2b) and counted with FeatureCounts (version 1.5). RNA-seq raw data was filtered using soft threshold: genes with less than 5 reads in more than 80% of samples were filtered out. RNA-seq data was then subjected to RLE normalization. RNA-seq data from public sources was preprocessed accordingly.

Both microarray and RNA-seq data were log-transformed and subjected to scaling. Gene IDs from all used platforms were mapped to Entrez IDs, while gene IDs mapping to multiple Entrez IDs were removed. Human genes were mapped to the corresponding one-to-one mouse orthologs, according to bioMart annotation. Only genes detected in at least 10% of samples were maintained, resulting in the final subset of 19,310 genes. Besides classical scaling-based normalization, we also tested normalizations based on ranking and YuGene transformation³³. For differential aging clock, random age group of healthy control animals was selected for each dataset. Median expression of every gene was calculated for this subset and subtracted from gene expression profiles for every sample. The resulting gene expression profiles were used as a training set for biological age prediction. Lifespan-adjusted age for every sample was calculated as a chronological age divided by maximum lifespan, defined as 90^{th} quantile lifespan of animals in the corresponding cohort. Survival curves for each animal model were extracted from public sources and used to calculate the maximum lifespan.

### Aging Clock Development

To develop a model of biological age prediction, we utilized supervised machine learning. We randomly selected 90% of samples as a training set and used the remaining 10% of samples as a test set. To identify the most accurate clock, we trained 4 types of models, including Elastic Net linear model, random forest, support vector machines (SVM) and gradient boosting (XGBoost). Models were trained to predict chronological or lifespan-adjusted age in linear or log-log-transformed scale (as described in⁹). Missed gene expression values were imputed based on median values for the corresponding genes calculated from training set. Hyperparameters were optimized with GroupKFold cross-validation on 5 folds, where source of each sample (GEO ID) was considered as a group. Mean Absolute Error (MAE) and R² were utilized as accuracy metrics during cross-validation and evaluation of final model. To obtain generalized estimate of model accuracy, we performed nested cross-validation, randomly dividing data into training and test sets 25 times. During each run, the model was trained on a training set, and its accuracy was estimated on a test set. This resulted in distribution of MAE and R² for each type of model and outcome variable transformation.

### Application of the Clock Model

Preprocessing of the data prior to applying the clock included filtering of low-expressed genes using soft threshold (genes with less than 5 reads in more than 80% of samples were removed), mapping of gene IDs to the mouse Entrez IDs, RLE normalization (for RNA-seq data), log-transformation, scaling and ranking/YuGene transformation (optionally, for the corresponding clock models). Missing values corresponding to the clock genes not detected in the data were imputed with the average values precalculated during the training of the clock model.

For single-cell RNA-seq datasets, prior to the described preprocessing pipeline, meta-cells were prepared by randomly pooling gene expression profiles across certain number of cells corresponding to the same cell type or tissues. Afterwards, the preprocessing algorithm was the same as for the bulk RNA-seq datasets.

Preprocessed gene expression profiles were used as an input for the clock, and tAges were returned by the model as an output. Differences between tAges of control and treated groups were assessed using linear regression model (for OSKM reprogramming datasets) and independent t-tests (for other applications). When multiple comparisons were assessed, obtained p-values were adjusted with Benjamini-Hochberg method.

### Example 1. Rodent Transcriptomic Clocks of Biological Age

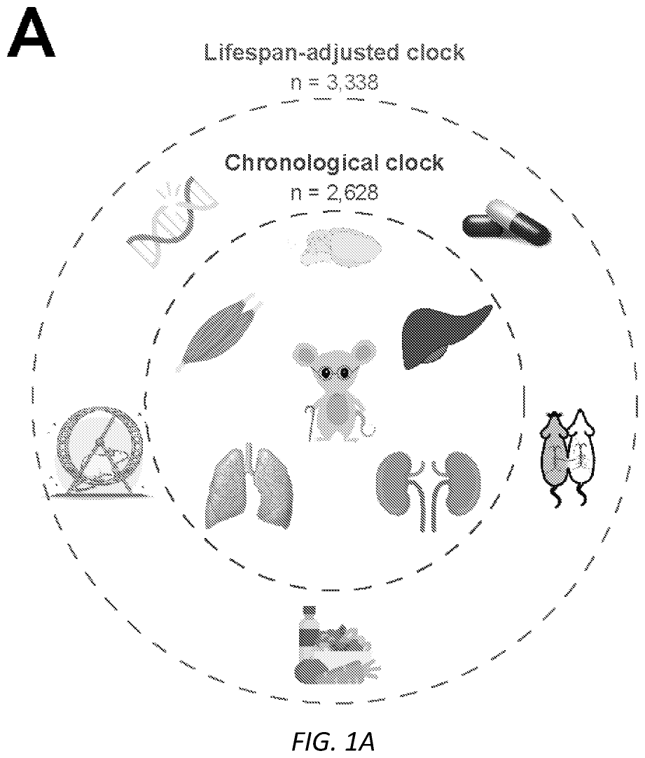

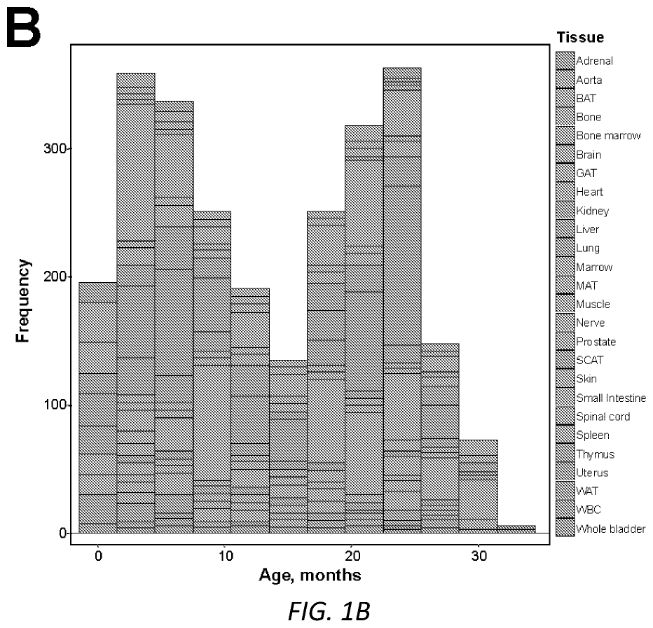

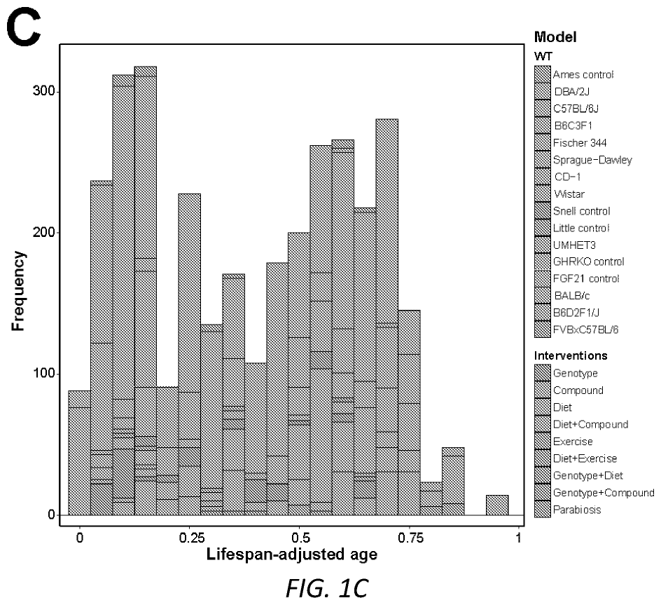

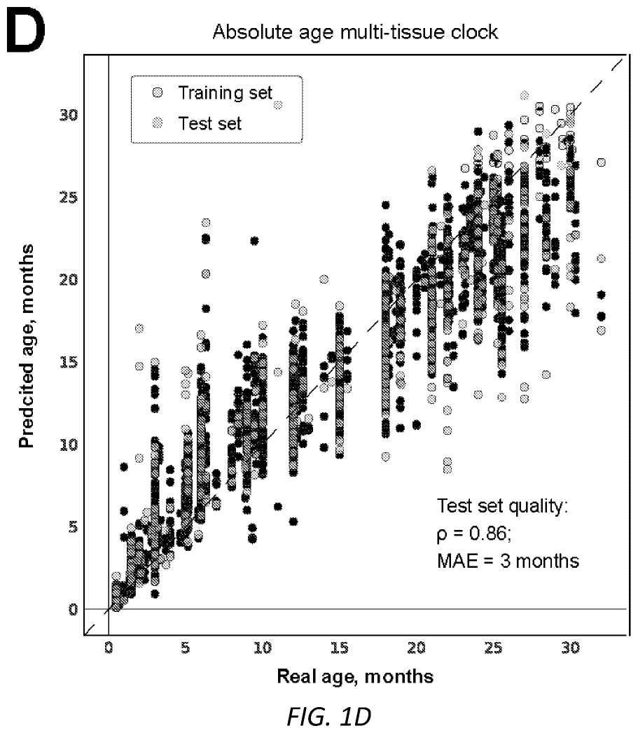

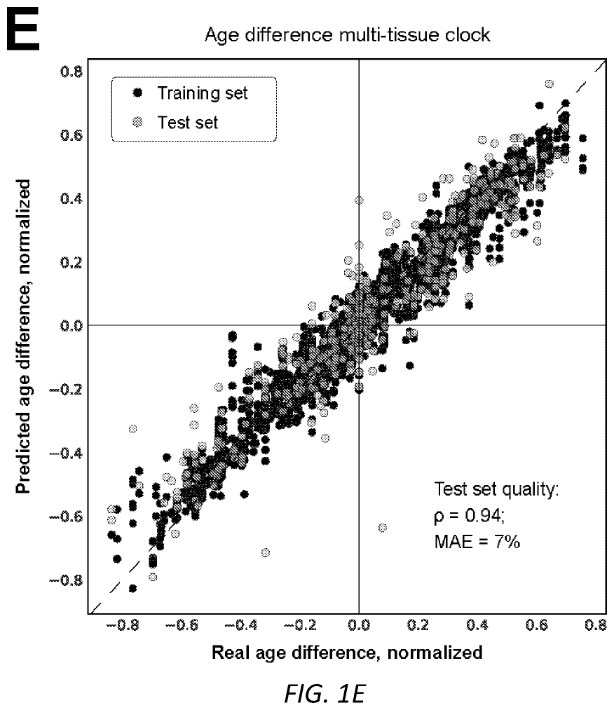

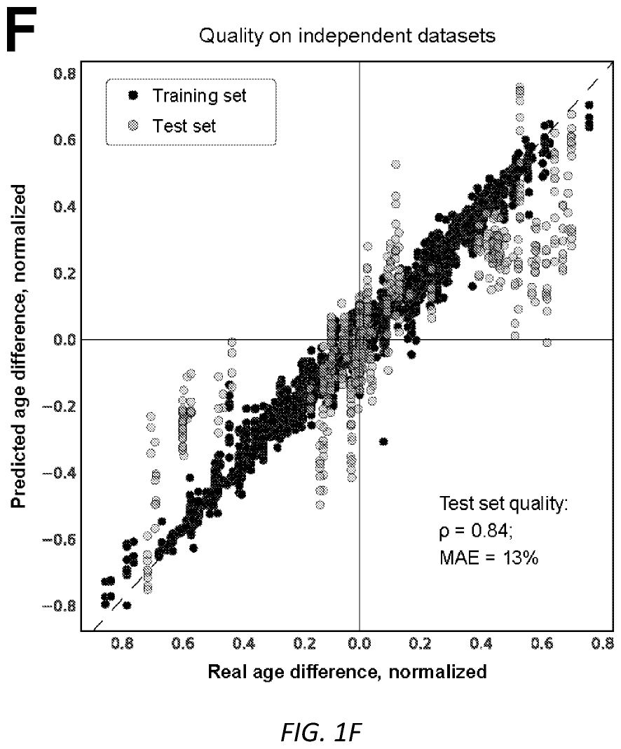

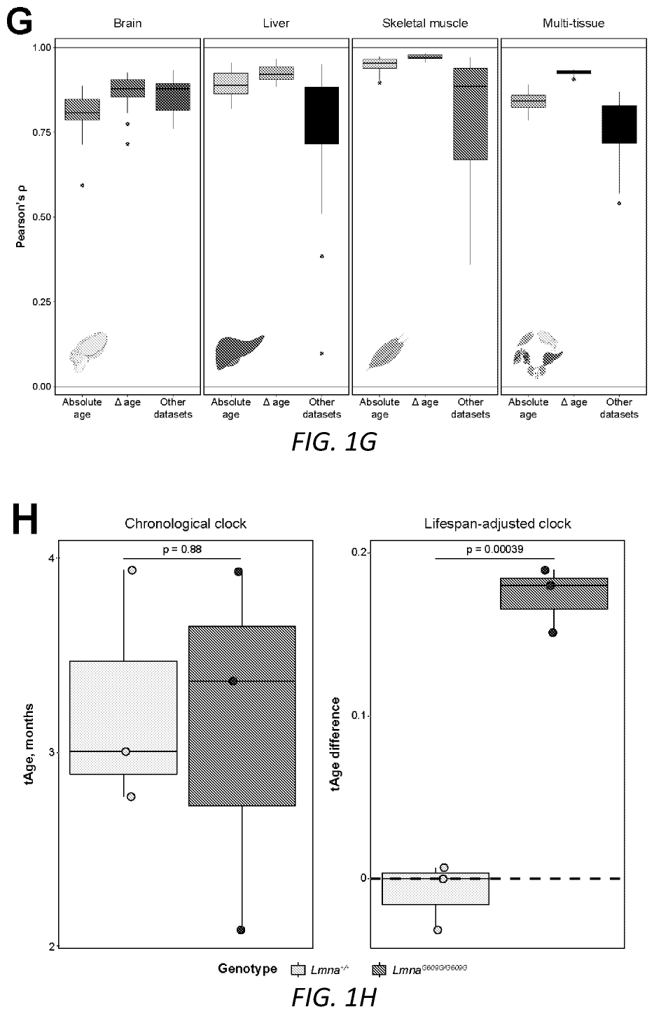

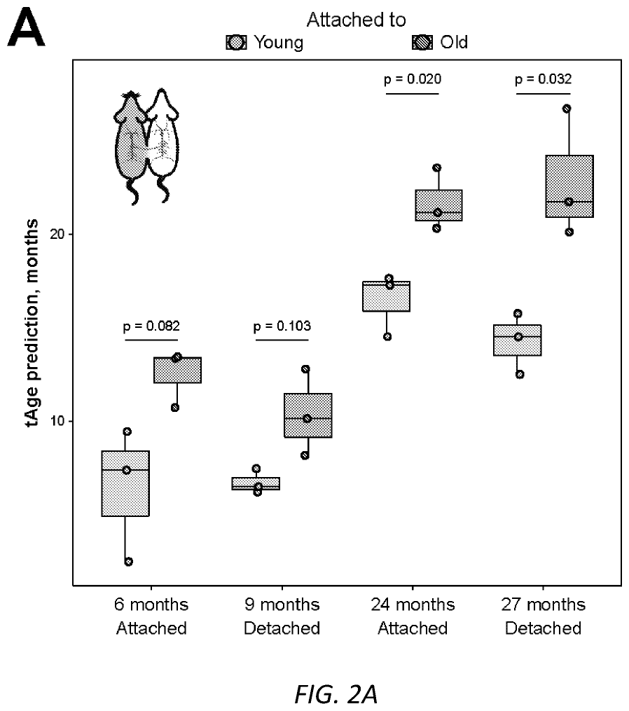

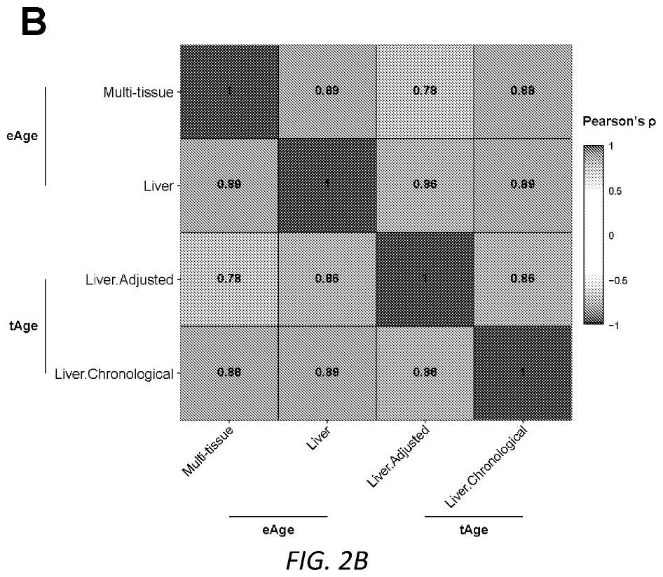

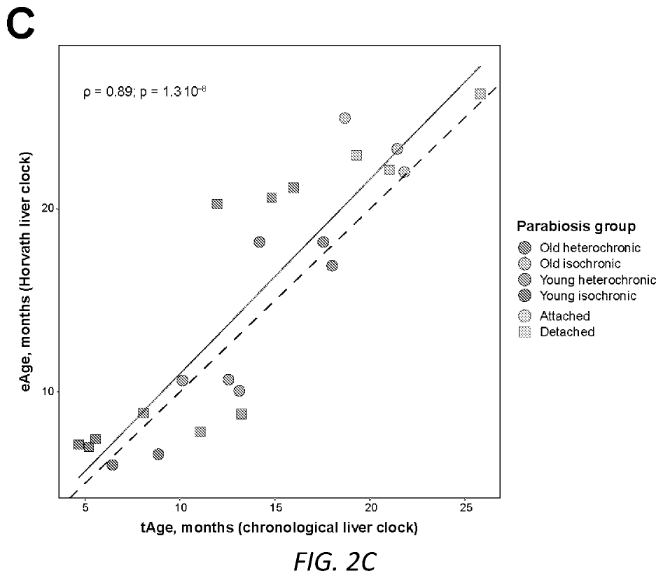

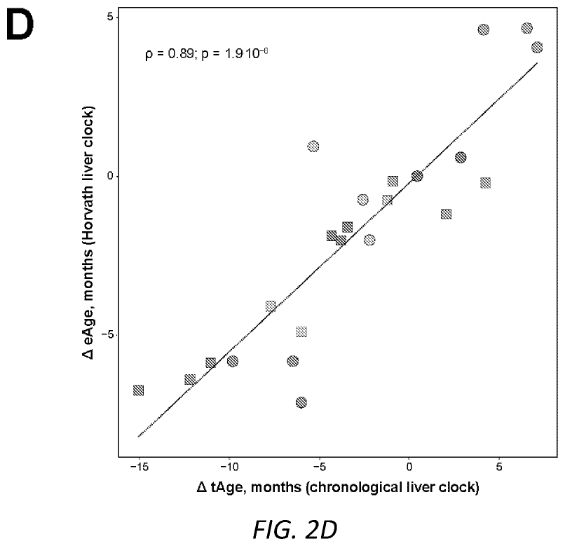

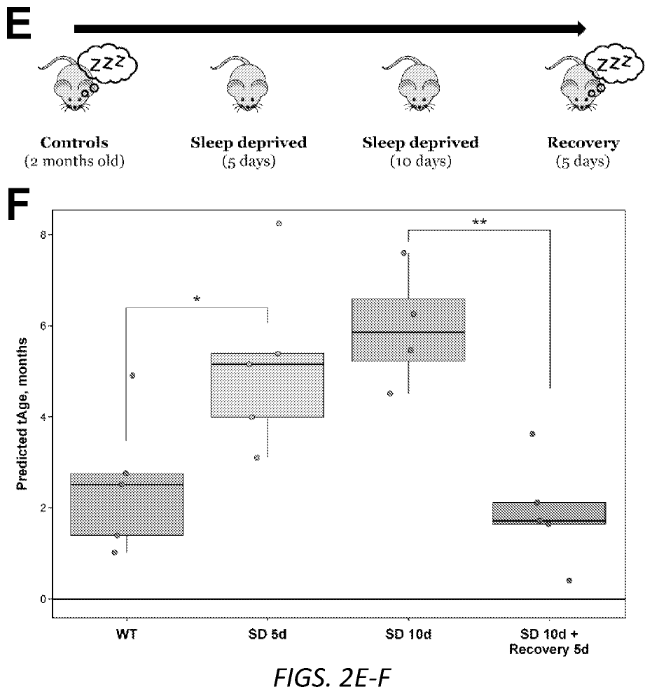

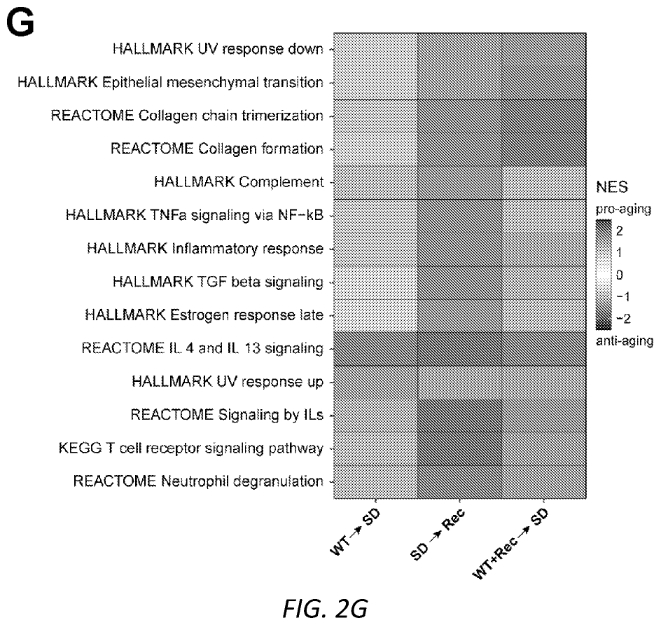

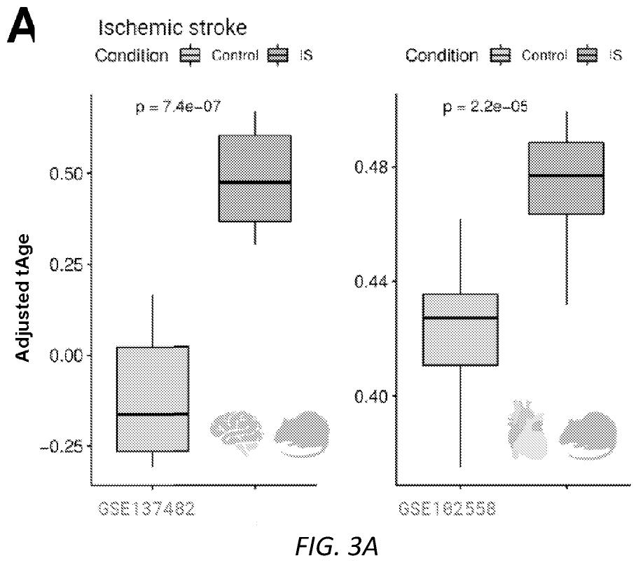

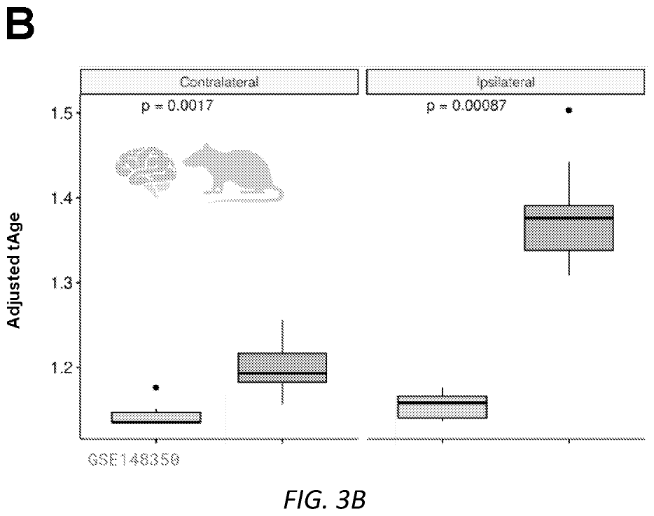

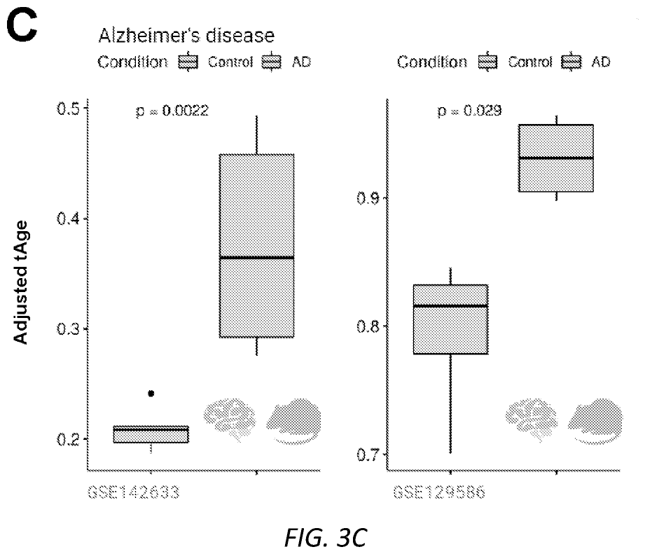

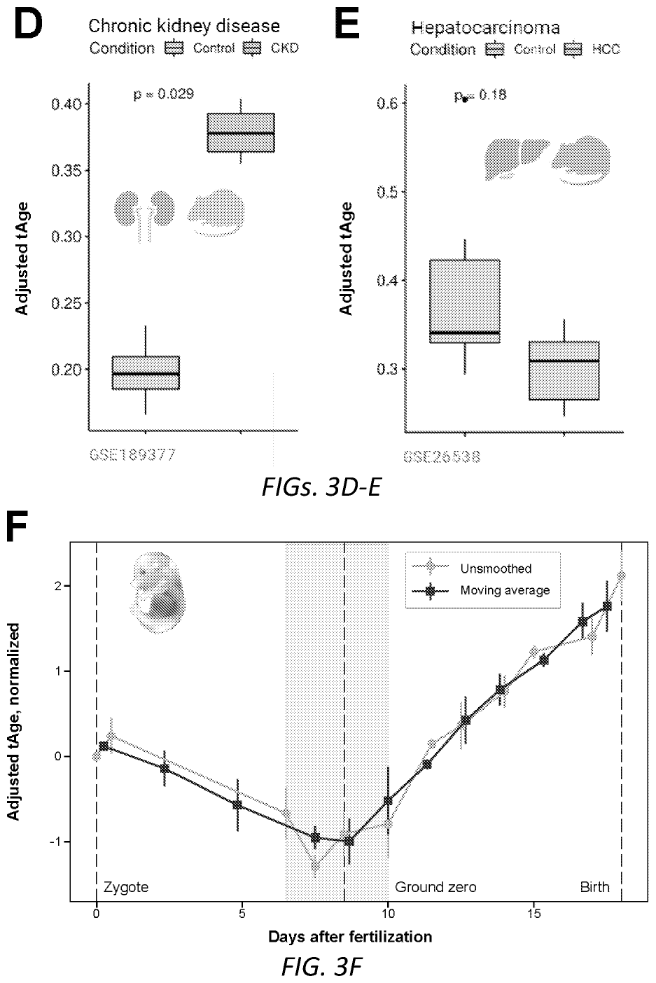

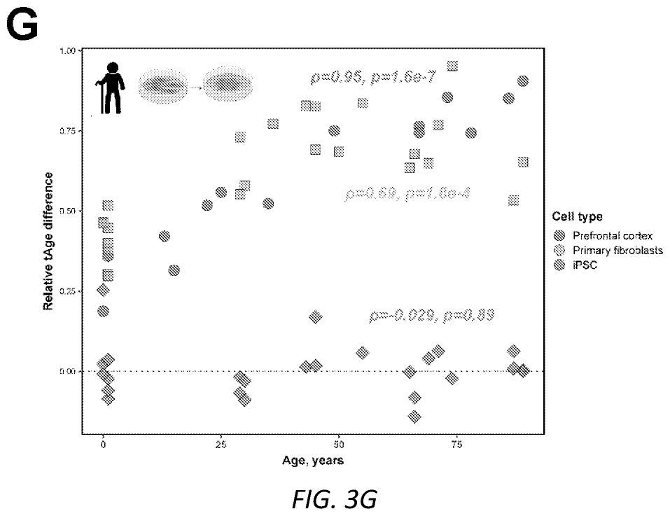

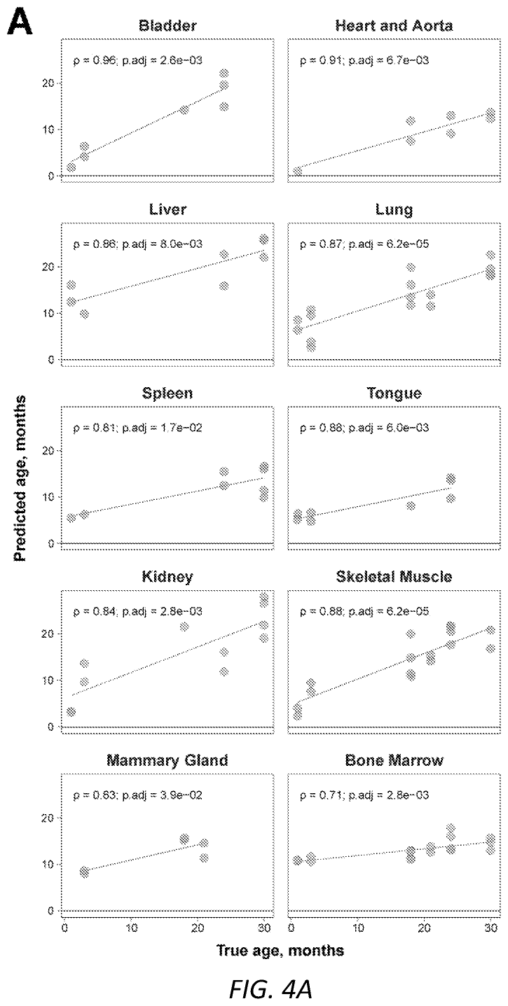

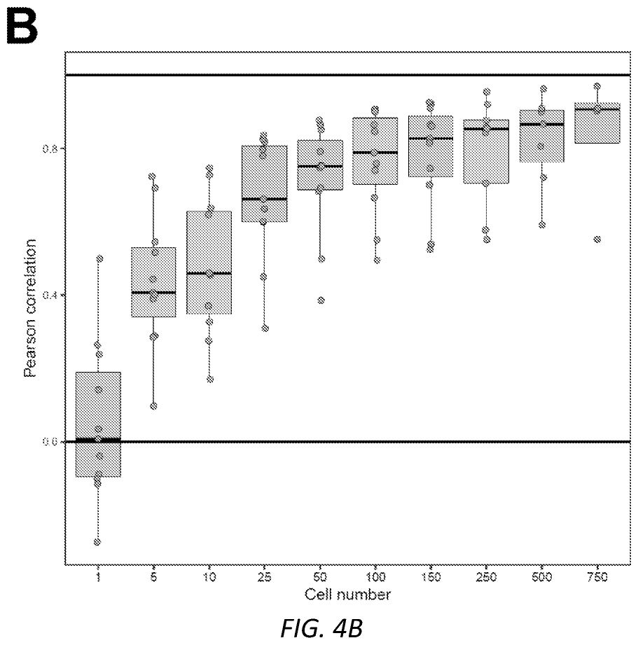

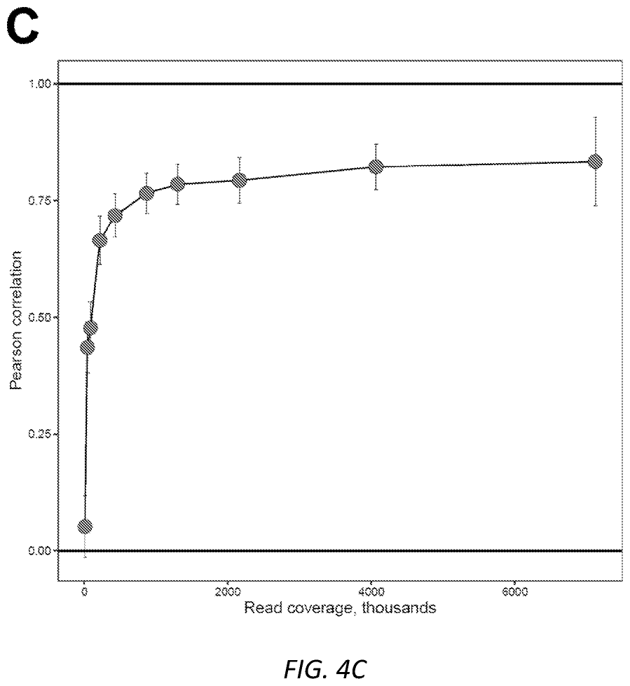

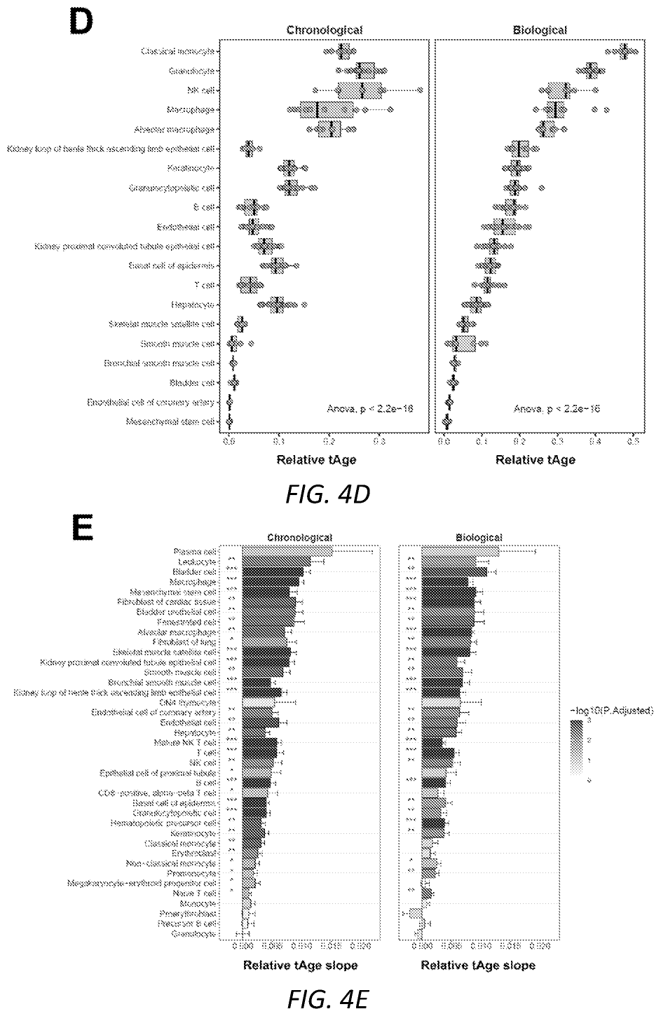

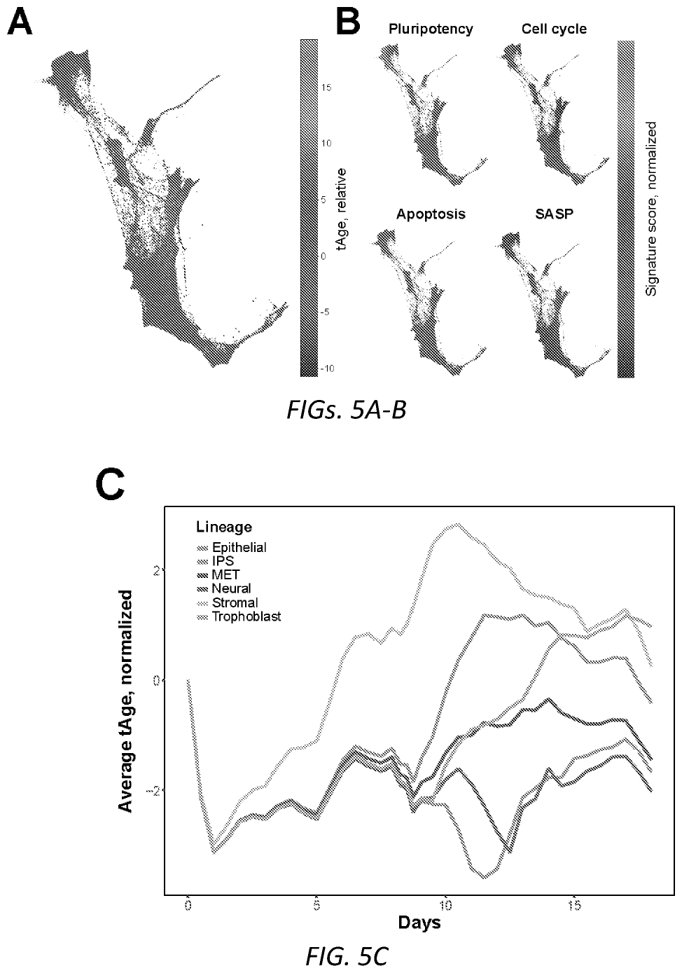

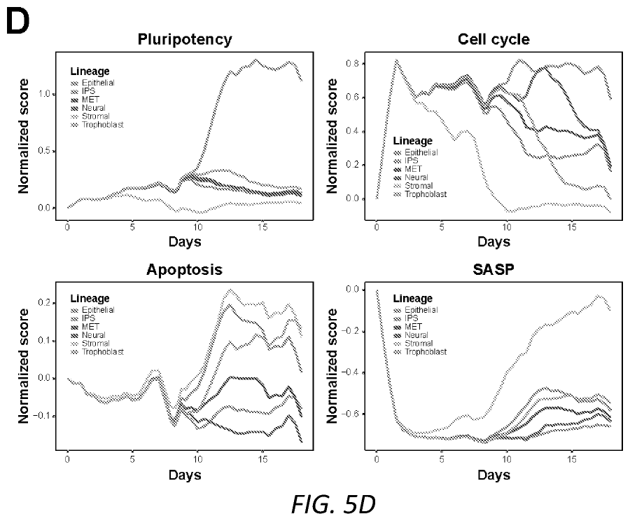

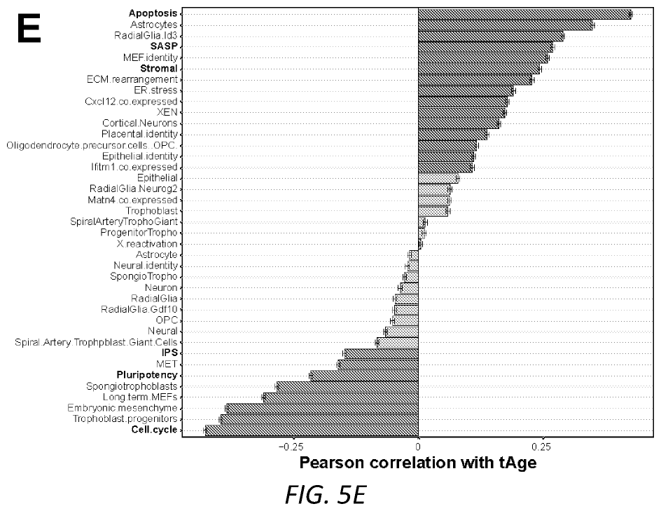

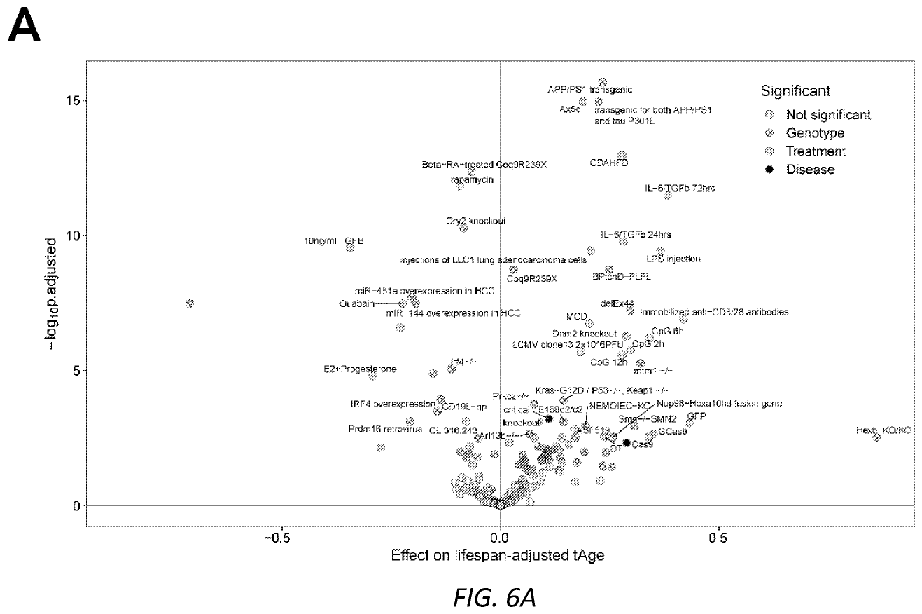

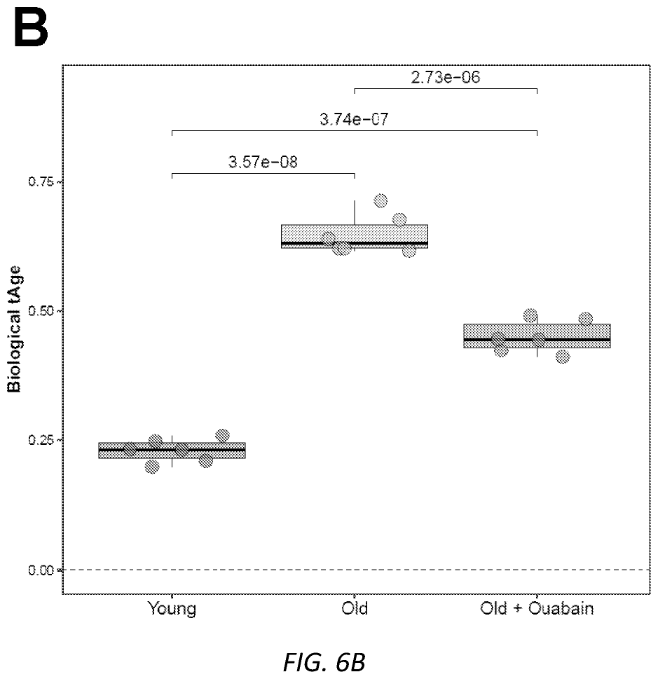

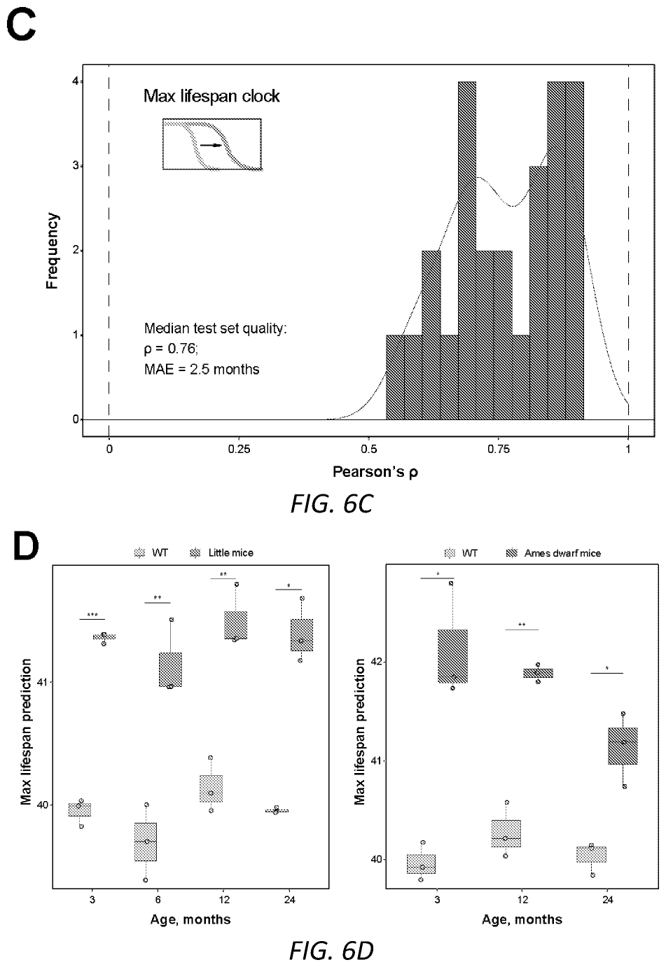

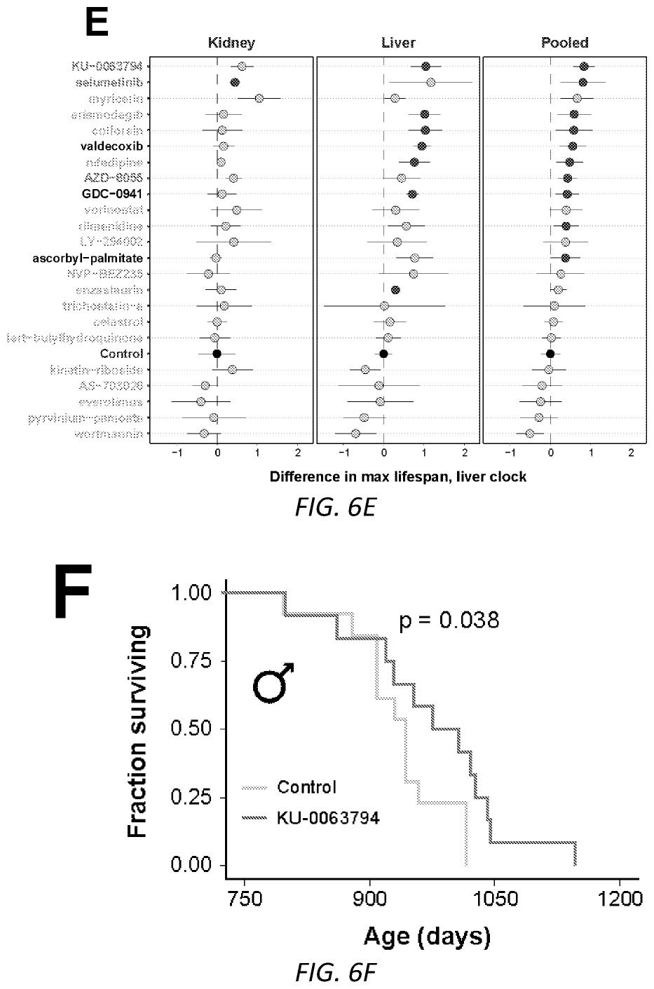

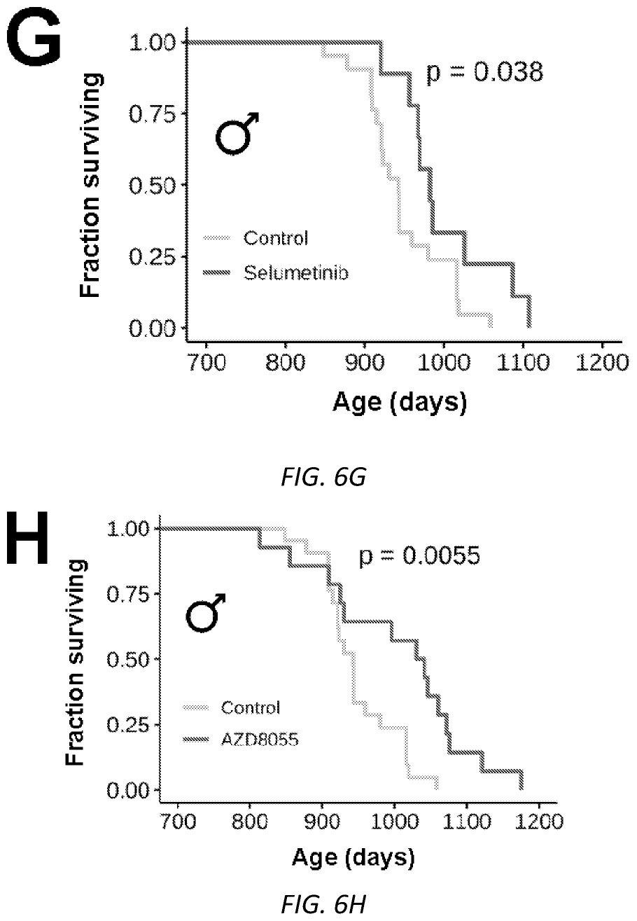

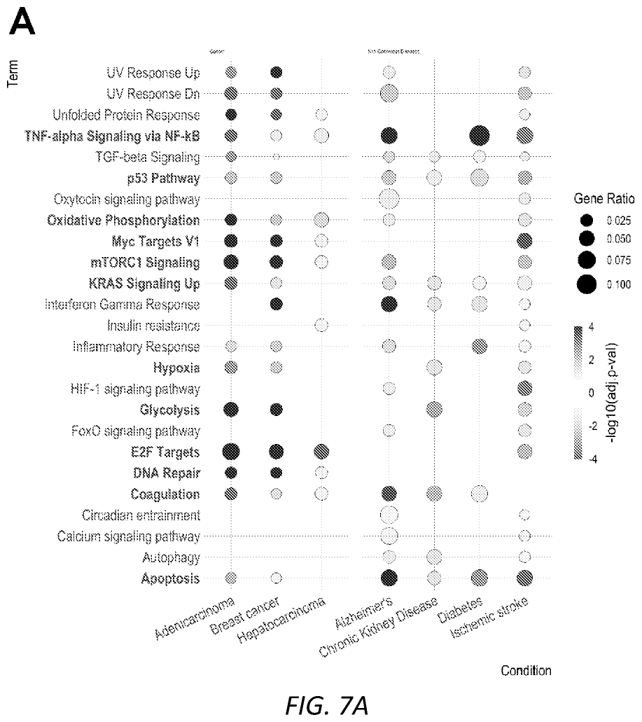

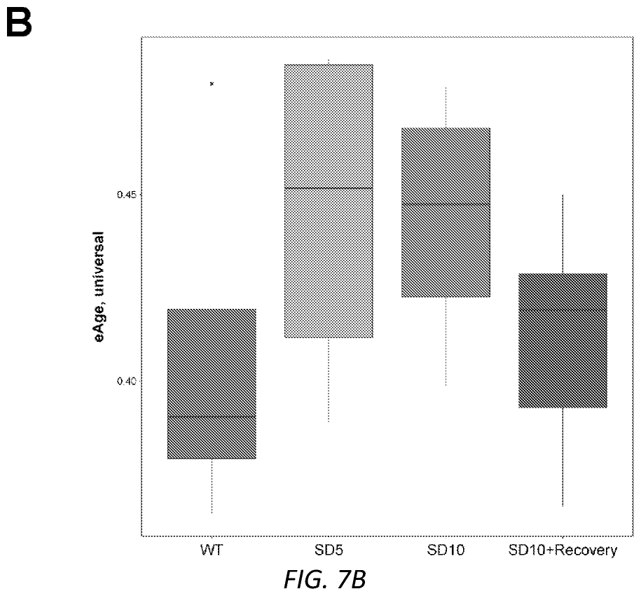

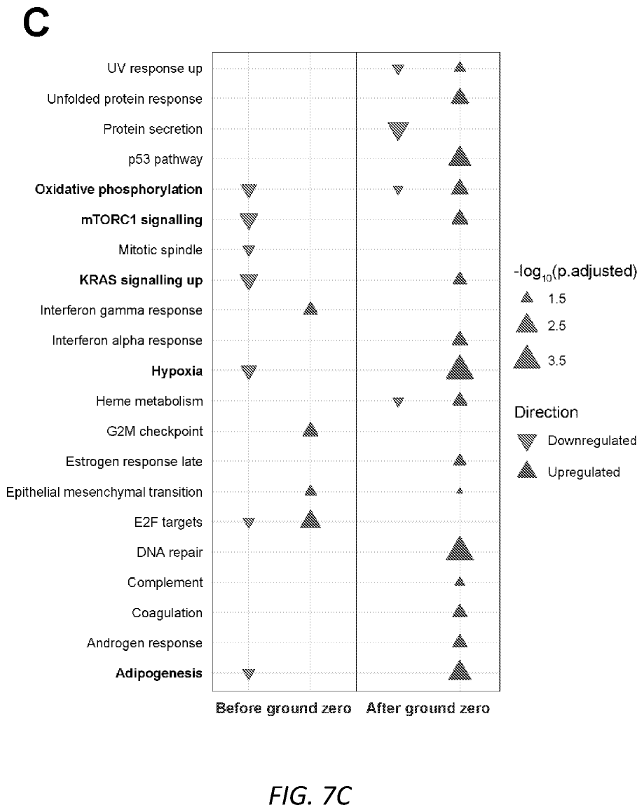

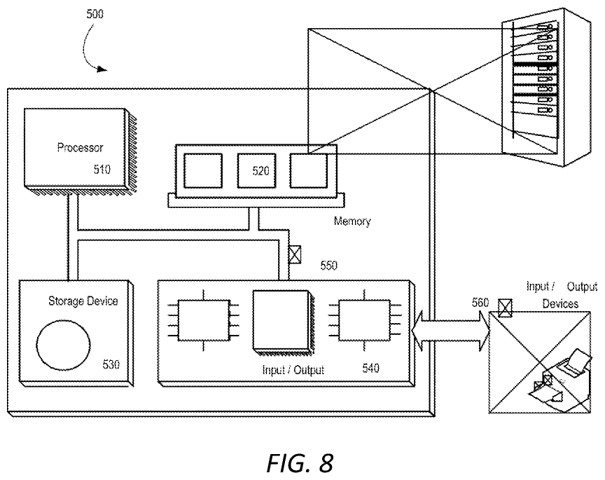

To construct transcriptomic clocks of chronological age in rodents, we aggregated gene expression data corresponding to 2,568 samples obtained from healthy mice and rats across 26 tissues from 84 independent datasets (FIG. 1A). To separate detrimental biomarkers of aging from potential neutral or beneficial age-related changes and to build a clock of biological aging, we also introduced 588 tissue samples corresponding to various genetic, pharmacological and environmental interventions that either extend or shorten mouse lifespan. We further added lifespan-extending interventions to this dataset by sequencing 182 liver samples of male and female mice subjected to 20 different chemical compounds tested by the ITP program¹⁻⁴, together with the age- and sex-matched controls. By normalizing the age of rodents by maximum lifespan of corresponding models obtained from survival studies, we calculated lifespan-adjusted age for each given sample, ranging from 0 (birth) to 1 (end of life). This resulted in 3,338 samples, for which both gene expression and lifespan-adjusted age were measured at once. The resulting data covered a substantial range of rodent lifespan, from 0.5 to 32 months, as well as various genetic backgrounds and models of lifespan-extending and -shortening interventions (FIG. 1B-C).

Using the collected data and response values of real and lifespan-adjusted age, we constructed clocks of chronological and biological age (Table A). For tissues highly represented in our dataset, such as liver, brain and skeletal muscle, we built tissue-specific clocks. We also constructed multi-tissue transcriptomic clocks using all the data across 26 tissues. To make our clocks robust across independent datasets, we utilized group K-fold approach, using independent datasets for training and cross-validation sets. We randomly assigned 15% of the sampling size to the test set and applied an elastic net algorithm, previously shown to be effective for the construction of epigenetic clocks⁶⁻⁹. Chronological liver, brain, muscle, and multi-tissue clocks were able to predict absolute real age of healthy mice with the Pearson's correlation coefficients of 0.93, 0.85, 0.9 and 0.86, respectively (FIG. 1D). Lifespan-adjusted clocks resulted in results of similar quality. To check if the obtained quality was robust across different subsets of training and test sets, we reproduced the algorithm using 25 independent random subsamplings. The median quality of the clocks estimated using Pearson's correlation coefficient was found to be 0.81, 0.89, 0.95 and 0.84 for brain, liver, muscle, and multi-tissue clock, respectively (FIG. 1G). To examine if this model can be expanded to human data, we also included 1,289 brain, skeletal muscle and skin samples from people of different ages as well as 10 brain samples from people with Hutchinson-Gilford progeria syndrome. The model trained on mouse, rat and human samples exhibited high quality on test set (Pearson's correlation coefficient >0.75), suggesting that a single transcriptomic clock can be applied to capture biological aging of tissues across multiple mammalian species, including human.

Since gene expression data is often accompanied by substantial batch effects, we tested whether clock quality was higher when age deviation instead of absolute age was predicted. To do that, for every dataset we randomly selected one control group of a certain age and calculated ages and gene expression values of all samples within this dataset compared to the age and median gene expression profile of this random group. By applying the same machine learning algorithm, we obtained clocks with significantly higher quality (FIG. 1E), resulting in median Pearson's correlation coefficients on test sets equal to 0.88, 0.92, 0.97 and 0.93, respectively (FIG. 1G). Linear model coefficients of top 500 genes with the highest absolute weights for trained models are stored in Table A.

To check how robust the clocks are when applied to independent datasets, we estimated their quality on test sets assembled from datasets that were not included in the training set (FIG. 1F). The resulting median Pearson's correlation coefficients of the clocks on independent datasets were equal to 0.88, 0.84, 0.89 and 0.8 for brain, liver, muscle, and multi-tissue clocks, respectively (FIG. 1G). Therefore, despite a significant batch effect and noise associated with gene expression data, our models were able to predict both chronological and biological age of samples obtained from independent datasets with high accuracy, comparable to the clocks based on other types of omics data^{6,10}.

Interestingly, despite high accuracy of real age prediction, the chronological transcriptomic aging clock not adjusted for lifespan was unable to capture the difference between livers of control mice and mice subjected to Hutchinson-Gilford progeria syndrome (HGPS) (FIG. 1H). Although mice with the progeria-associated mutations in Lmna gene have 10-fold reduced lifespan compared to wildtype mice, they are not predicted to be older by many chronological clocks, including epigenetic clocks⁶. However, our lifespan-adjusted clocks of biological age were able to capture the difference and predicted a significantly higher transcriptomic age (tAge) for HGPS mice (FIG. 1H), suggesting that they are indeed more applicable for the assessment of the effect of interventions on healthspan than regular clocks trained on chronological aging.

### Example 2. Consistency Between the Predictions of Transcriptomic and Epigenetic Clocks

To check if the predictions of our transcriptomic clocks were consistent with those estimated by the established epigenetic clocks, we utilized the data on mice subjected to isochronic and heterochronic parabiosis¹¹. In this dataset, we carried out RNA-seq and used DNA methylation data from the same mice representing 4 groups: young (3-month-old) mice attached to young mice, old (21-month-old) mice attached to young mice, young mice attached to old mice, and old mice attached to young mice. After 3 months of attachment, mice were sacrificed, and their livers were collected. Besides, livers were collected from mice attached for 3 months, followed by detachment for 2 months. Previously, we observed a significant rejuvenation of old mice attached to the young ones compared to the isochronic group, that was preserved even after 2 months of detachment¹¹. On the other hand, young mice attached to the old mice were older, but mostly recovered after detachment.

Interestingly, the same pattern was observed when our transcriptomic liver clocks were applied (FIG. 2A). Moreover, the Pearson's correlation coefficient between age estimates obtained with liver epigenetic clocks and liver transcriptomic clocks was 0.89 (FIG. 2C). Remarkably, the correlation between tAge and epigenetic age (eAge) remained the same even after chronological age was subtracted from the predictions (FIG. 2B,D). Residuals of tAge and eAge (Δ Ages) were strongly correlated for chronological epigenetic clocks as well as liver transcriptomic clocks, both chronological and lifespan-adjusted (FIG. 2B). Therefore, epigenetic and transcriptomic clocks provide consistent results even after adjustment for chronological age, pointing to shared molecular mechanisms of biological aging at the level of DNA methylation and gene expression.

### Example 3. Transcriptomic Clocks Capture Age-Related Changes Induced by Diseases, Sleep Deprivation, Cell Reprogramming and Embryogenesis

To determine if our clocks capture the effect of other established models that affect biological age, we applied them to the gene expression data from rodents subjected to various aging-related conditions and diseases as well as to control rodents of the same chronological age. We observed a consistent increase in biological age in mice subjected to ischemic stroke, both in brain and heart, as assessed using lifespan-adjusted brain and multi-tissue clocks, respectively (FIG. 3A). Interestingly, we observed higher difference between control and diseased mice for samples collected from ipsilateral brain hemisphere than for samples from the contralateral hemisphere, although statistically significant difference was observed in both cases (FIG. 3B).

We also detected significantly higher biological tAge in brains of 5xFAD mice subjected to the rodent model of Alzheimer's disease (AD) (FIG. 3C). The same pattern of increased biological tAge was observed in kidneys of mice with chronic kidney disease (CKD) and type II diabetes (FIG. 3D). Interestingly, contrary to the other age-related diseases, hepatocarcinoma resulted in a slight non-significant decrease of tAge in murine liver (FIG. 3E).

To investigate functional processes behind the observed difference in biological transcriptomic age, we utilized functional enrichment analyses (FIG. 7A) looking for pathways enriched for genes, which expression is responsible for the observed increase of tAge (and decreased in case of carcinoma). We found significant upregulation of genes associated with apoptosis, p53 signaling, coagulation and NF-kB signaling related to inflammation, shared by the majority of diseases including ischemic stroke, AD, CKD and diabetes. On the other hand, carcinoma samples were characterized by the downregulation of aging-associated coagulation and NF-kB signaling genes. Therefore, the constructed clocks allow to investigate particular cellular mechanisms that lie behind observed age-related changes.

To examine biological age of animals subjected to short-term detrimental interventions, we employed sleep deprivation (SD) model (FIG. 2E). We subjected 2-month-old mice to 5 days of SD, 10 days of SD or 10 days of SD followed by 5 days of recovery, and compared their transcriptomic ages estimated in liver to those from age-matched control animals. Transcriptomic clocks revealed increase of biological age in response to 5 or 10 days of sleep deprivation, and it was restored to the original level after 5-day recovery (FIG. 2F). Interestingly, epigenetic clocks applied to the same tissues also showed similar trend towards age acceleration driven by sleep deprivation (FIG. 7B), but it was not significant. In contrast, transcriptomic clocks demonstrated a statistically significant difference between the groups (adjusted p-value <0.05), suggesting that gene expression aging-related changes may be more sensitive to short-term interventions compared to changes in DNA methylation. Functional enrichment analysis revealed that the increase of biological age caused by SD was mainly associated with the upregulation of genes involved in inflammatory response, interleukin and TGF-β signaling, while these changes were reversed after 5 days of recovery (FIG. 2G).

Previously described epigenetic clocks demonstrated U-shaped eAge behavior during early murine embryogenesis, reaching the minimum eAge approximately between 6.5 and 10.5 days after fertilization¹². This finding was consistent with the ground zero model stating that different molecular systems of the embryo are rejuvenated during early embryogenesis, followed by monotonous aging of the animal¹³. To test if this model is confirmed at the level of gene expression, we applied the multi-tissue lifespan-adjusted transcriptomic clock to the gene expression data ranging from the zygote to the 17^{th} day of embryogenesis¹⁴. Consistent with the epigenetic data, we observed U-shaped dynamics of biological tAge during early development with the minimum value approximately between 7 and 10 days after fertilization (FIG. 3F) that agrees with the ground zero estimate measured based on DNA methylation data. Functional enrichment revealed that some of the pathways are both responsible for the decrease of tAge before ground zero and the increase of tAge after it, including TNFa signaling via NF-kB, p53 signaling, apoptosis, hypoxia and cholesterol homeostasis (FIG. 7C).

To test if our multi-tissue transcriptomic clock trained on mouse and rat tissues can be applied to assess biological age of human samples, we evaluated transcriptomic age of prefrontal cortex and primary fibroblasts derived from people of various chronological ages (FIG. 3G). Transcriptomic clocks correctly predicted age differences across these individuals and were especially accurate for prefrontal cortex (Pearson's ρ=0.95 and 0.69 for prefrontal cortex and primary fibroblasts, respectively; p-value <2·10⁻⁴). Interestingly, biological age of OSKM-induced pluripotent stem cells (iPSCs) derived from fibroblasts of these individuals was significantly lower than tAge of original cells and wasn't correlated with the chronological age of donors (Pearson's ρ=−0.029, p-value=0.89). This is consistent with the previous findings, showing that epigenetic age of iPSCs is close to 0 regardless of the original chronological age of cells subjected to OSKM reprogramming. Using multi-tissue transcriptomic clock, we also showed that dynamics of rejuvenation associated with classical OSKM reprogramming is different between mouse and human cells¹⁵. In murine cells, stable minimum tAge was reached approximately after 10 days of OSKM induction. In addition, transcriptomic rejuvenation of human cells followed sigmoidal curve and took more time, reaching minimum approximately on the 28^{th} day of reprogramming. Interestingly, such rejuvenation dynamics is consistent with differences in the duration of reprogramming in mouse and human cells, lasting, on average, for 14 and 30 days in mouse and human cell lines, respectively¹⁶. Besides, our clock also captured rejuvenation in mouse and human cells subjected to chemical reprogramming with 7c cocktail^{17,18}, demonstrating further consistency across predictions of epigenetic and transcriptomic clocks.

### Example 4. Transcriptomic Clocks Predict Chronological Aging of Single Cells

To examine if multi-tissue transcriptomic clocks built on bulk data can predict chronological age of individual cells, we applied them to murine scRNA-seq data collected by Tabula Muris Consortium¹⁹. This dataset covers single cell gene expression data from various tissues of mice of different age and sex. To increase coverage of single cell data, we utilized a meta-cell approach and pooled read counts from randomly selected 500 cells for every biological sample. Following this procedure, multi-tissue chronological clock was able to capture chronological age dynamics for most mouse organs (FIG. 4A) with median Pearson's ρ=0.87. By repeating the same procedure taking different number of cells used for meta-cell data aggregation, we found that 50 cells were sufficient to achieve median accuracy of Pearson's ρ=0.75 across organs (FIG. 4B). This number of cells corresponds to the average coverage of approximately 430,000 reads per meta-cell (FIG. 4C).

To examine if biological age varies across different cell types, we aggregated meta-cells from randomly selected 50 cells for individual cell types from 1-month-old mice. We observed significant variation of tAge across cell types, both for chronological and lifespan-adjusted clocks (FIG. 4D). Several types of white blood cells, including monocytes, granulocytes and macrophages, had higher biological age, in agreement with their role in inflammaging and development of aging-related diseases^{20,21}. In contrast, mesenchymal stem cells exhibited the lowest transcriptomic age, presumably associated with the decreased proportion of stem cells with age and detrimental effect of stem cell exhaustion on organism's healthspan^{22,24}.

We also assessed age-related change of transcriptomic age for each cell type presented in Tabula Muris dataset, utilizing meta-cell procedure, multi-tissue transcriptomic clock and linear regression model. Remarkably, for most cell types we observed significant positive tAge slope (FIG. 4E), suggesting that aging is accompanied by systemic increase of biological age in the majority of cells. Interestingly, even mesenchymal stem cells exhibited significant increase of tAge with age. Therefore, these transcriptomic clocks were able to capture both age-related changes in cell composition of the organ (e.g., accumulation of macrophages and granulocytes) as well as aging-associated changes in gene expression within individual cells.

### Example 5. Transcriptomic Clocks Reveal Aging-Related Dynamics of Single Cells During Reprogramming

To check if the gene expression clock of biological age can capture trajectory of age-related dynamics during OSKM-induced cellular reprogramming, we utilized scRNA-seq data, covering 18 days of reprogramming of murine embryonic fibroblasts (MEFs)²³. We applied lifespan-adjusted multi-tissue transcriptomic clock to each individual cell presented in the data and observed significant variability of tAge across different cell trajectories (FIG. 5A). Using gene expression signatures of pluripotency, cell cycle, apoptosis and senescence-associated secretary phenotype (SASP), we found that some of them characterized cell types with higher average tAge (FIG. 5B). Thus, stromal cells characterized by upregulation of SASP- and apoptosis-associated genes had also higher tAge, whereas mesenchymal-epithelial transition (MET) and induced pluripotent cells (IPS) showed increased expression of cell cycle genes and lower tAge.

To focus on the dynamics of transcriptomic age throughout trajectory of different cell types generated during OSKM reprogramming, we applied an optimal transport method²³. We observed a significant drop of tAge during first days of reprogramming, consistent with the mouse bulk reprogramming data (FIG. 5C). Interestingly, after that biological age was significantly increased in cells moving towards stromal and epithelial trajectory, while some other cell types, including IPS, neural and MET cells, maintained lower average tAge. Remarkably, ancestors of stromal cells demonstrated a similar significant increase of SASP-associated gene expression during first days of reprogramming, while apoptosis-related genes were upregulated in stromal, epithelial and trophoblast cell types (FIG. 5D), pointing to the impact of apoptosis and SASP pathways on the variation of tAge across different cell types. To test it, we estimated correlation between tAge and different gene expression signatures across all individual cells (FIG. 5E). Indeed, we observed strong positive correlation between tAge and signatures of apoptosis and SASP, and negative correlation between tAge and signatures of cell cycle and pluripotency. This finding suggests that cell reprogramming doesn't lead to systemic rejuvenation of all cells. Instead, it results in a significant variation of biological age across cell types, leading to a decrease of tAge in some of the cells (IPS, MET), but significant upregulation of aging-related biomarkers in the others (stromal, epithelial cells). Single-cell data and transcriptomic clocks allow deeper investigation of aging-related changes induced by different interventions and assessment of heterogeneity of biological age dynamics induced by these models.

### Example 6. Gene Expression Clocks of Aging and Lifespan Predict New Longevity Interventions

To discover candidate interventions that slow down biological aging and extend mammalian lifespan and healthspan, we utilized ClockBase²⁵ and processed 1,349 publicly available RNAseq datasets corresponding to mouse tissues exposed to various pharmacological, dietary and genetic interventions. For every intervention, we used gene expression data to calculate tAges of subjected mice and compared them with tAges of age-matched control mice. This resulted in identification of a number of interventions with a statistically significant effect on tAge (FIG. 6A). As expected, we observed an increase of biological age in transgenic models of Alzheimer's disease and in mice subjected to LPS injection, interleukin 6 injection and choline-deficient, L-amino acid-defined, high-fat diet (CDAHFD) associated with development of nonalcoholic steatohepatitis. Interestingly, we also identified interventions leading to a decrease in biological age. In addition to the well-known lifespan-extending interventions, such as rapamycin, they included β3-adrenoceptor agonist CL 316,243^{26,27} and the cardiac glycoside ouabain²⁸. According to the transcriptomic clock, 3-month ouabain treatment resulted in an ˜20% reduction of biological age in 24-month-old mice (FIG. 6B). This compound was also shown to exhibit senolytic activity and improve physical function of aged rodents²⁸. Our data suggests that ouabain may counteract age-related changes and produce lifespan-extending effect in mammals.

To examine if longevity of rodents can be predicted based on gene expression data, we used 1,030 liver samples of mice and rats with known maximum lifespan to construct transcriptomic clock of expected lifespan. Using an elastic net model and group K-fold cross-validation, we built a clock that predicted expected maximum lifespan of the given animal with a median Pearson's correlation coefficient of 0.76 and median MAE of 2.5 months, as assessed using 25 randomly subsampled test sets (FIG. 6C). Remarkably, the introduction of chronological age to the feature space didn't improve the model, suggesting that the transcriptome profile covers most of the aging-associated effect on longevity. Linear model coefficients of top 500 genes with the highest absolute weights for absolute lifespan and differential lifespan clocks are stored in Table A.

We applied the constructed clock to the liver samples obtained from long-lived growth hormone (GH) deficient mutant mice of different ages together with the corresponding controls²⁹. Predicted expected lifespans of both Ames dwarf mice (Prop1^{df/df}) and Little mice (Ghrhr^{lit/lit}) were indeed significantly higher than those of control mice (FIG. 6D). The gene expression clock captured the difference in lifespan between the models across presented ages, suggesting that certain longevity-associated molecular mechanisms may be preserved throughout the lifespan.

To test if the constructed clock can be used to identify new longevity interventions, we applied it to the liver and kidney samples obtained from young UM-HET3 male mice subjected to 25 different compounds for 1 month together with age-matched controls. These compounds were chosen using a CMAP platform³⁰ and gene expression signatures of longevity identified in our previous studies³¹. 23 of the 25 compounds induced differential expression of at least 1 gene (adjusted p-value <0.05) and were selected for subsequent analysis. Transcriptomic clock revealed a significant positive effect for 10 of these compounds on expected lifespan of mice (adjusted p-value <0.05 for data pooled across tissues) (FIG. 6E).

To validate the predictions obtained with transcriptomic longevity clock, we selected 6 of these compounds, including KU-0063794 (an mTOR inhibitor), selumetinib (a MEK1/2 inhibitor), valdecoxib (a nonsteroidal anti-inflammatory drug (NSAID)), AZD-8055 (an mTOR inhibitor), GDC-0941 (pictilisib, a PI3Kα/δ inhibitor), and ascorbyl-palmitate, and subjected 20-24 25-month-old C57BL/6JNia mice to each of these compounds added to the animal diet. Remarkably, 3 out of 6 compounds predicted to be beneficial for mouse longevity according to the clock indeed significantly extended lifespan of old male mice (log-rank p-value <0.05) (FIGS. 6F-H). These drugs included MEK 1/2 inhibitor selumetinib and mTOR inhibitors KU-0063794 and AZD-8055, suggesting that the obtained model may indeed be applied to identify novel longevity interventions. We further tested the effect of KU-0063794 on mouse healthspan and found that this compound improved gait speed and decreased frailty index of 30-month-old mice³², suggesting that the methods described herein can be applied to identify novel compounds that extend both lifespan and healthspan of old animals.

### REFERENCES

1. Strong, R., Miller, R. A., Antebi, A., Astle, C. M., Bogue, M., Denzel, M. S., Fernandez, E., Flurkey, K., Hamilton, K. L., Lamming, D. W., et al. (2016). Longer lifespan in male mice treated with a weakly estrogenic agonist, an antioxidant, an a-glucosidase inhibitor or a Nrf2-inducer. Aging Cell 15, 872-884. 10.1111/acel.12496.
2. Miller, R. A., Harrison, D. E., Allison, D. B., Bogue, M., Debarba, L., Diaz, V., Fernandez, E., Galecki, A., Garvey, W. T., Jayarathne, H., et al. (2020). Canagliflozin extends life span in genetically heterogeneous male but not female mice. JCI Insight 5, e140019.
3 Strong, R., Miller, R. A., Bogue, M., Fernandez, E., Javors, M. A., Libert, S., Marinez, P. A., Murphy, M. P., Musi, N., Nelson, J. F., et al. (2020). Rapamycin-mediated mouse lifespan extension: Late-life dosage regimes with sex-specific effects. Aging Cell 19, 1-10. 10.1111/acel. 13269.
4 Harrison, D. E., Strong, R., Reifsnyder, P., Kumar, N., Fernandez, E., Flurkey, K., Javors, M. A., Lopez-Cruzan, M., Macchiarini, F., Nelson, J. F., et al. (2021). 17-a-estradiol late in life extends lifespan in aging UM-HET3 male mice; nicotinamide riboside and three other drugs do not affect lifespan in either sex. Aging Cell 20, 1-10. 10.1111/acel.13328.
5 Siler, W. (1983). Parameters of mortality in human populations with widely varying life spans. Stat. Med. 2, 373-380.
6. Horvath, S. (2013). DNA methylation age of human tissues and cell types. Genome biol 14, R115. 10.1186/gb-2013-14-10-r115.
7. Petkovich, D. A., Podolskiy, D. I., Lobanov, A. V, Lee, S., Miller, R. A., and Gladyshev, V. N. (2017). Using DNA Methylation Profiling to Evaluate Biological Age and Longevity Interventions. Cell Metab. 25, 954-960.e6. 10.1016/j.cmet.2017.03.016.
8 Meer, M. V, Podolskiy, D. I., and Tyshkovskiy, A. (2018). A whole lifespan mouse multi-tissue DNA methylation clock. Elife 7, 1-16. 10.7554/eLife.40675.
9 Lu, A. T., Quach, A., Wilson, J. G., Reiner, A. P., Aviv, A., Raj, K., Hou, L., Baccarelli, A. A., Li, Y., Stewart, J. D., et al. (2019). DNA methylation GrimAge strongly predicts lifespan and healthspan. Aging (Albany. NY). 11, 303-327.
10. Lu, A. T., Fei, Z., Haghani, A., Robeck, T. R., Zoller, J. A., Li, C. Z., Lowe, R., Yan, Q., Zhang, J., Vu, H., et al. (2023). Universal DNA methylation age across mammalian tissues. Nat. Aging. 10.1038/s43587-023-00462-6.
11. Zhang, B., Lee, D. E., Trapp, A., Tyshkovskiy, A., Lu, A. T., Bareja, A., Kerepesi, C., Mckay, L. K., Shindyapina, A. V, Dmitriev, S. E., et al. (2023). Multi-omic rejuvenation and life span extension on exposure to youthful circulation. Nat. Aging. 10.1038/s43587-023-00451-9.
12. Kerepesi, C., Zhang, B., Lee, S.-G., Trapp, A., and Gladyshev, V. N. (2021). Epigenetic clocks reveal a rejuvenation event during embryogenesis followed by aging. Sci. Adv. 7, eabg6082.
13. Gladyshev, V. N. (2021). The Ground Zero of Organismal Life and Aging. Trends Mol. Med. 27, 11-19. 10.1016/j.molmed.2020.08.012.
14. Xue, L., Cai, J. Y., Ma, J., Huang, Z., Guo, M. X., Fu, L. Z., Shi, Y. B., and Li, W. X. (2013). Global expression profiling reveals genetic programs underlying the developmental divergence between mouse and human embryogenesis. BMC Genomics 14. 10.1186/1471-2164-14-568.
15. Kriukov, D., Khrameeva, E. E., Gladyshev, V. N., Dmitriev, S. E., and Tyshkovskiy, A. (2022). Longevity and rejuvenation effects of cell reprogramming are decoupled from loss of somatic identity. bioRxiv. 10.1101/2022.12.12.520058.
16. Fu, K., Chronis, C., Soufi, A., Bonora, G., Edwards, M., Smale, S. T., Zaret, K. S., Plath, K., and Pellegrini, M. (2018). Comparison of reprogramming factor targets reveals both species-specific and conserved mechanisms in early iPSC reprogramming. BMC Genomics 19, 1-13. 10.1186/s12864-018-5326-1.
17. Yang, J., Petty, C. A., Dixon-McDougall, T., Lopez, M. V., Tyshkovskiy, A., Maybury-lewis, S., Tian, X., Ibrahim, N., Chen, Z., Griffin, P. T., et al. (2023). Chemically induced reprogramming to reverse cellular aging. Aging (Albany. NY). 15, 5966-5989.
18. Mitchell, W., Goeminne, L. J. E., Tyshkovskiy, A., Zhang, S., Paulo, J. A., Pierce, K. A., Choy, A. H., Clish, C. B., Gygi, S. P., and Gladyshev, V. N. (2023). Multi-omics characterization of partial chemical reprogramming reveals evidence of cell rejuvenation. bioRxiv.
19. Almanzar, N., Antony, J., Baghel, A. S., Bakerman, I., Bansal, I., Barres, B. A., Beachy, P. A., Berdnik, D., Bilen, B., Brownfield, D., et al. (2020). A single-cell transcriptomic atlas characterizes ageing tissues in the mouse. Nature 583, 590-595. 10.1038/s41586-020-2496-1.
20. Kristof, V. A., Strecker, J.-K., Tulotta, C., Minnerup, J., Schulz, C., and Soehnlein, O. (2023). Neutrophils in aging and aging-related pathologies. Immunol. Rev. 314, 357-375. 10.1111/imr.13153.
21. Maeyer, R. P. H. De, and Chambers, E. S. (2020). The impact of ageing on monocytes and macrophages. Immunol. Lett. 230, 1-10. 10.1016/j.imlet.2020.12.003.
22. Lopez-Otin, C., Blasco, M. A., Partridge, L., Serrano, M., and Kroemer, G. (2013). The hallmarks of aging. Cell 153, 1194-1217. 10.1016/j.cell.2013.05.039.
23. Schiebinger, G., Shu, J., Tabaka, M., Cleary, B., Subramanian, V., Solomon, A., Gould, J., Liu, S., Lin, S., Berube, P., et al. (2019). Optimal-Transport Analysis of Single-Cell Gene Expression Identifies Developmental Trajectories in Reprogramming. Cell 176, 928-943.e22. 10.1016/j.cell.2019.01.006.
24. Lopez-Otin, C., Blasco, M. A., Partridge, L., Serrano, M., and Kroemer, G. (2023). Hallmarks of aging: An expanding universe. Cell 186, 243-278. 10.1016/j.cell.2022.11.001.
25. Ying, K., Tyshkovskiy, A., Trapp, A., Liu, H., Moqri, M., Kerepesi, C., and Gladyshev, V. N. (2023). ClockBase: a comprehensive platform for biological age profiling in human and mouse. bioRxiv.
26. Himms-hagen, J., Taatjes, J., Lang, S. S., Claus, H., Taatjes, J., and Lang, S. S. (1994). Effect of CL-316,243, a thermogenic b3-agonist, on energy balance and brown and white adipose tissues in rats. Am. J. Physiol. 266, R1371-82.
27. Wang, W., Ishibashi, J., Trefely, S., Shao, M., Cowan, A. J., Sakers, A., Lim, H., Connor, S. O., Doan, M. T., Cohen, P., et al. (2019). A PRDM16-driven metabolic signal from adipocytes regulates precursor cell fate. Cell Metab. 30, 174-189. 10.1016/j.cmet.2019.05.005.A.
28. Guerrero, A., Herranz, N., Sun, B., Wagner, V., Gallage, S., Guiho, R., Wolter, K., Pombo, J., Irvine, E. E., Innes, A. J., et al. (2019). Cardiac glycosides are broad-spectrum senolytics. Nat. Metab. 1, 1074-1088. 10.1038/s42255-019-0122-z.
29. Amador-Noguez, D., Yagi, K., Venable, S., and Darlington, G. (2004). Gene expression profile of long-lived Ames dwarf mice and Little mice. Aging Cell 3, 423-441. 10.1111/j.1474-9728.2004.00125.x.
30. Subramanian, A., Narayan, R., Corsello, S. M., Peck, D. D., Natoli, T. E., Lu, X. L., Gould, J., Doench, J. G., Bittker, J. A., Root, D. E., et al. (2017). A Next Generation Connectivity Map: L1000 platform and the first 1,000,000 profiles. Cell 171, 1437-1452. 10.1016/j.cell.2017.10.049.
31. Tyshkovskiy, A., Bozaykut, P., Borodinova, A. A., Gerashchenko, M. V., Ables, G. P., Garratt, M., Khaitovich, P., Clish, C. B., Miller, R. A., and Gladyshev, V. N. (2019). Identification and Application of Gene Expression Signatures Associated with Lifespan Extension. Cell Metab. 30, 573-593. 10.1016/j.cmet.2019.06.018.
32. Tyshkovskiy, A., Ma, S., Shindyapina, A. V, Tikhonov, S., Lee, S.-G., Bozaykut, P., Castro, J. P., Seluanov, A., Schork, N. J., Gorbunova, V., et al. (2023). Distinct longevity mechanisms across and within species and their association with aging. Cell 186, 1-21. 10.1016/j.cell.2023.05.002.
33. Edgar, R. (2002). Gene Expression Omnibus: NCBI gene expression and hybridization array data repository. Nucleic Acids Res. 30, 207-210. 10.1093/nar/30.1.207.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A computer-implemented method for calculating expected total and remaining lifespan, chronological age, and lifespan-adjusted age of a biological test system, optionally a cell, tissue, organ, or organism, the method comprising:
providing a biological test system, optionally a cell, tissue, organ, or organism;
determining expression of two or more transcripts identified in Table A;
accessing, from memory, a model for calculating lifespan, chronological age, or lifespan-adjusted age of a biological test system, wherein the model calculates lifespan, chronological age, or lifespan-adjusted age, i.e. chronological age of the organism normalized by its expected lifespan, based on expression of the two or more transcripts; and
calculating, using the model, expected lifespan, chronological age, or lifespan-adjusted age for the system.

2. The method of claim 1, comprising determining expression of 5, 10, 20, 30, 40, 50, 60, 70, 80, 90, 100, 125, 150, or more of the transcripts.

3. The method of claim 2, comprising determining expression of up to 100, 125, 150, 175, 200, 250, 300, 350, 400, or 500 of the transcripts.

4. The method of claim 1, further comprising applying an intervention to the system, determining expression of the one or more transcripts during and/or after an application of an intervention, and calculating chronological age, lifespan-adjusted age, or expected lifespan based on the expression of the one or more transcripts.

5. The method of claim 4, further comprising comparing the chronological age, lifespan-adjusted age, or expected lifespan to a reference chronological age, lifespan-adjusted age, or expected lifespan.

6. The method of claim 5, wherein the reference chronological age, lifespan-adjusted age, or expected lifespan is a baseline chronological age, lifespan-adjusted age, or expected lifespan obtained in the same test system before application of an intervention, or a chronological age, lifespan-adjusted age, or expected lifespan obtained earlier in time in the same test system, or a chronological age, lifespan-adjusted age, or expected lifespan in a reference system that represents the chronological age, lifespan-adjusted age, or expected lifespan in the absence of an intervention.

7. The method of claim 1, wherein chronological age, lifespan-adjusted age, or expected lifespan is calculated using a software-based modeling algorithm, optionally wherein the algorithm comprises a linear algorithm; principal component analysis (PCA); classification or decision trees; elastic net analysis; linear and polynomial support vector machines (SMV); shrunken centroids; random forest algorithms; support vector machines; gradient boosting; or neural networks.

8. The method of claim 7, comprising calculating a chronological age, lifespan-adjusted age, or expected lifespan using the determined expression and applying an algorithm to the levels.

9. The method of claim 8, wherein the algorithm comprises:
tAge=intercept+b1*T1+b2*T2+ . . . +bn*Tn;
where b1-bn are the pre-trained model coefficients for genes listed in Table A, and
T1-Tn are the normalized expression levels of given Transcripts.

10. The method of claim 9, wherein the model is a log-log model, and the method further comprises performing a transformation wherein:
tAgeₘ=10**(−10**(−tAge)),
wherein tAge is the estimate from the formula above, and tAgeₘ is a transformed tAge that reflects the chronological or lifespan-adjusted biological age on a scale of 0-1, where 0 is a moment of birth, and 1 is a maximum achievable lifespan for the model.

11. The method of claim 1, further comprising identifying an intervention as having a positive effect when expected lifespan increases, and/or chronological age, or lifespan-adjusted age decreases, and/or identifying an intervention as having a damaging effect when expected lifespan decreases, and/or chronological age, or lifespan-adjusted age increases.

12. The method of claim 11, further comprising:
selecting an intervention that has been identified as having a positive effect as a candidate intervention;
applying the candidate intervention to an in vivo model of, optionally wherein the model is a non-human test animal or a human subject with a disorder or condition associated with aging; and
determining whether the candidate intervention has a positive effect on the model, optionally on the disorder or condition related to aging.

13. A method of predicting an effect of an intervention on lifespan, the method comprising:
providing a biological test system, optionally a cell, tissue, organ, or organism;
determining expression of two or more transcripts identified in Table A;
applying an intervention to the system,
calculating chronological age, lifespan-adjusted age, or expected lifespan using the method of claim 1;
comparing the predicted chronological age, lifespan-adjusted age, or expected lifespan to a reference chronological age, lifespan-adjusted age, or expected lifespan; and
identifying an intervention as likely to have a protective effect on lifespan when chronological age or lifespan-adjusted age decreases and/or expected lifespan increases, and/or identifying an intervention as likely to have a damaging effect on lifespan when chronological age or lifespan-adjusted age increases and/or expected lifespan decreases.

14. The method of claim 13, comprising determining expression of 5, 10, 20, 30, 40, 50, 60, 70, 80, 90, 100, 125, 150, or more of the transcripts.

15. The method of claim 14, comprising determining expression of up to 100, 125, 150, 175, 200, 250, 300, 350, 400, 500, or 1,000 transcripts, including determining expression of at least 5, 10, 20, 30, 40, 50, 60, 70, 80, 90, 100, 125, 150, or all of the transcripts.

16. The method of claim 13, wherein the reference chronological age, lifespan-adjusted age or expected lifespan is a baseline chronological age, lifespan-adjusted age or expected lifespan obtained in the same test system before application of an intervention, or a chronological age, lifespan-adjusted age or expected lifespan obtained earlier in time in the same test system, or a chronological age, lifespan-adjusted age or expected lifespan in a reference system that represents the chronological age, lifespan-adjusted age or expected lifespan in the absence of an intervention.

17. The method of claim 13, comprising calculating chronological age, lifespan-adjusted age or expected lifespan using an algorithm, optionally wherein the algorithm comprises using a manual or software-based modeling algorithm, optionally wherein the algorithm comprises a linear algorithm; principal component analysis (PCA); classification or decision trees; elastic net analysis; linear and polynomial support vector machines (SMV); shrunken centroids; random forest algorithms; support vector machines; gradient boosting; or neural networks.

18. The method of claim 17, comprising calculating a chronological age, lifespan-adjusted age or expected lifespan using the determined expression and applying an algorithm to the levels.

19. The method of claim 18, wherein the algorithm comprises:
tAge=intercept+b1*T1+b2*T2+ . . . +bn*Tn
where b1-bn are the pre-trained model coefficients for every gene from Table A,
and T1-Tn are the normalized expression levels of given Transcripts.

20. The method of claim 20, wherein the model is a log-log model, and the method further comprises performing a transformation wherein:
tAgeₘ=10**(−10**(−tAge)),
wherein tAge is the estimate from the formula above, and tAgeₘ is a transformed tAge that reflects the chronological or lifespan-adjusted biological age on a scale of 0-1, where 0 is a moment of birth, and 1 is a maximum achievable lifespan for the model.

21. The method of claim 20, further comprising:
selecting an intervention that has been identified as having a protective effect as a candidate intervention;
applying the candidate intervention to an in vivo model of a disorder or condition associated with aging, optionally wherein the model is a non-human test animal or a human subject in a clinical trial, optionally a model or a trial of a disorder or condition associated with aging; and
determining whether the candidate intervention has a protective effect on the model.
